(19) United States
(12) Patent Application Publication
(10) Pub. No.: US 2002/0049223 A1
(43) Pub. Date: Apr. 25, 2002

(54) Quinoline and naphthyridine carboxylic acid antibacterials

(76) Inventors: Steven W. Elmore, Gurnee, IL (US); Curt S. Cooper, Vernon Hills, IL (US); Colleen C. Schultz, Chicago, IL (US); Douglas K. Hutchinson, Antioch, IL (US); Pamela L. Donner, Mundelein, IL (US); Brian E. Green, Wonder Lake, IL (US); David D. Anderson, Kenosha, WI (US); Qinghua Xie, Libertyville, IL (US); Jurgen Dinges, Grayslake, IL (US); Linda M. Lynch, Pleasant Prarie, WI (US); John K. Pratt, Kenosha, WI (US)

Correspondence Address: Steven F. Weinstock, ABBOTT LABORATORIES, D-377 / AP6D-2, 100 Abbott Park Road, Abbott Park, IL, 60064-6050, US

(21) Appl. No.: 09/850,664

(22) Filed: May 7, 2001

Related U.S. Application Data
(63) Continuation-in-part of application No. 09/705,332, filed on Nov. 3, 2000.
(60) Provisional application No. 60/163,920, filed on Nov. 5, 1999.

(51) Int. Cl. C07D 471/02; C 07D 4 1/02; A61K 31/4745; A61K 31/4709

(52) U.S. Cl. 514/300; 546/156; 514/312; 546/123

(57) ABSTRACT

Compounds having formula (I)

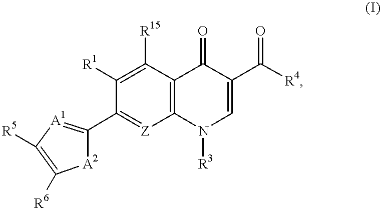

or pharmaceutically acceptable salts or prodrugs thereof, are useful as antibacterial agents.

This application is a continuation-in-part of copending U.S. provisional application Ser. No. 60/163,920, filed Nov. 5, 1999.

### TECHNICAL FIELD

This invention relates to quinoline and naphthyridine carboxylic acid derivatives having antibacterial properties, processes for making of the compounds, methods of treatment using the compounds and pharmaceutical compositions containing the compounds. Specifically, it relates to quinoline and naphthyridine carboxylic acid derivatives having a carbon-carbon linkage at the C-7 position of the molecule.

### BACKGROUND OF THE INVENTION

Many compounds having the quinoline carboxylic acid nucleus are known in the art for their antibacterial activity in curing infectious diseases. Some of the known antibiotics on the market to-date include, for example, ciprofloxacin (U.S. Pat. No. 4,670,444), norfloxacin (U.S. Pat. No. 4,146,719), enoxacin (U.S. Pat. No. 4,352,803), and tosufloxacin (U.S. Pat. No. 4,704,459). Most of these known quinoline carboxylic acid antibacterial compounds have a carbon-nitrogen linkage at the C-7 position of the quinoline nucleus.

There is a continuing need for discovering compounds which are more effective against resistant bacteria, have improved intestinal absorption, metabolic stability, and exhibit less phototoxicity and cytotoxicity.

### SUMMARY OF THE INVENTION

In one embodiment of the invention are disclosed compounds of formula (I)

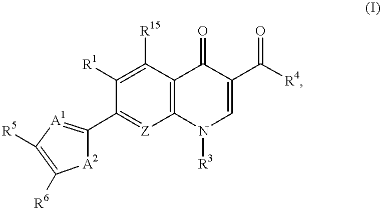

or pharmaceutically acceptable salts or prodrugs thereof, wherein

A¹ is nitrogen or

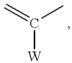

wherein W is selected from the group consisting of

(1) hydrogen and

(2) optionally substituted alkyl;

A² is selected from the group consisting of

(1) —S—,

(2) —O—, and

(3) —N(R⁷)—, wherein R⁷ is hydrogen or C₁-C₆ alkyl;

R¹ and R¹⁵ are independently selected from the group consisting of

(1) hydrogen,

(2) optionally substituted alkyl,

(3) halide,

(4) nitro, and

(5) optionally protected amino;

Z is nitrogen or

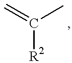

wherein R² is selected from the group consisting of

(1) hydrogen,

(2) optionally substituted alkyl,

(3) halide,

(4) optionally protected hydroxyl,

(5) —OR⁸, and

(6) —S(O)ₙR⁸, wherein n is zero, one, or two, and

wherein R⁸ in (4) and (5) is selected from the group consisting of

(a) C₃-C₆ alkenyl,

(b) C₁-C₆ alkyl, and

(c) C₃-C₆ alkynyl,

wherein (a)-(c) can be optionally substituted with one, two, or three substituents independently selected from the group consisting of

(i) alkoxy,

(ii) aryl,

(iii) C₃-C₆ cycloalkyl,

(iv) azido,

(v) cyano,

(vi) halide,

(vii) optionally protected amino,

(viii) optionally protected carboxyl, and

(ix) optionally protected hydroxyl;

R³ is selected from the group consisting of

(1) C₃-C₆ alkenyl,

(2) C₁-C₆ alkyl,

(3) C₃-C₆ alkynyl,

wherein (1)-(3) can be optionally substituted with one, two, or three substutuents independently selected from the group consisting of

(a) C₁-C₆ alkanoyloxy,

(b) C₁-C6 alkoxy,

(c) aryl,

(d) azido,

(e) cyano,

(f) C₃-C₆ cycloalkyl,

(g) halide,

(h) optionally protected amino,

(i) optionally protected carboxyl,

(j) optionally protected hydroxyl,

(k) oxo,

(l) C₁-C₆ perfluoroalkoxy,

(m) C₁-C₆ perfluorothioalkoxy, and

(n) thioxo,

(4) aryl,

(5) C₃-C₆ cycloalkyl, and

(6) heterocycle,

wherein (4)-(6) can be optionally substituted with one, two, or three substituents independently selected from the group consisting of

(a) C₁-C₆ alkanoyloxy,

(b) C₁-C₆ alkoxy,

(c) C₂-C₆ alkenyl,

(d) C₁-C₆ alkyl,

(e) C₂-C₆ alkynyl,

(f) aryl,

(g) azido,

(h) cyano,

(i) C₃-C₆ cycloalkyl,

(j) halide,

(k) optionally protected amino

(l) optionally protected carboxyl,

(m) optionally protected hydroxyl,

(n) C₁-C₆ perfluoroalkoxy, and

(o) C₁-C₆ perfluorothioalkoxy;

or

R² and R³ together are selected from the group consisting of

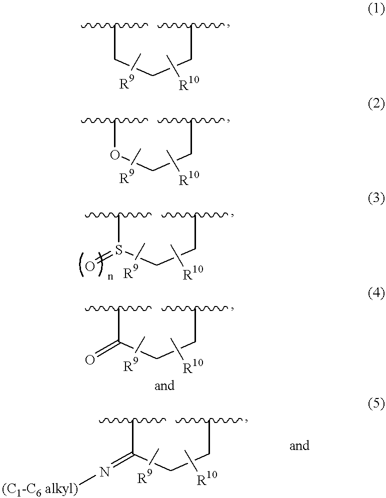

wherein one of R⁹ or R¹⁰ in (1)-(5) is hydrogen and the other is selected from the group consisting of

(1) hydrogen,

(2) C₁-C₆ alkyl,

(3) C₁-C₆ haloalkyl, and

(4) optionally protected hydroxyl,

or

wherein R⁹ and R¹⁰ together are alkylidene or C₃-C₆ spiroalkyl;

R⁴ is hydrogen or —OR¹¹, wherein R¹¹ is hydrogen or a carboxyl protecting group; and

R⁵ and R⁶ together are a carbocyclic or a heterocyclic ring, wherein the carbocyclic ring and the heterocyclic ring can be optionally substituted with one, two, or three substituents independently selected from the group consisting of

(1) optionally substituted aryl,

(2) azido,

(3) carboxaldehyde,

(4) cyano,

(5) halide,

(6) nitro,

(7) optionally substituted Cₗ-C₆ alkyl,

(8) optionally substituted C₃-C₆ alkenyl,

(9) optionally substituted C₃-C₆ alkynyl,

(10) optionally protected amino,

(11) optionally protected hydroxyl,

(12) optionally protected carboxyl,

(13) optionally substituted C₁-C₆ alkanoyloxy,

(14) optionally substituted C₁-C₆ alkoxy,

(15) optionally substituted aryl,

(16) optionally substituted C₃-C₆ cycloalkyl,

(17) optionally substituted heterocycle,

(18) oxo,

(19) C₁-C₆ perfluoroalkoxy,

(20) C₁-C₆ perfluorothioalkoxy,

(21) optionally substituted C₁-C₆ thioalkoxy,

(22) thioxo,

(23) a nitrogen protecting group,

(24) heterocycle,

(25) —C(O)N(R¹²)₂,

(26) —C(O)SR¹²,

(27) —N(R¹²)₂

(28) ═N—,

(29) —OC(O)N(R¹²)₂,

(30) ═N—N(R¹²)₂,

(31) ═N(R¹²)—N(R¹²)₂,

(32) —N(R¹²)—C(═NR¹²)—N(R¹²)₂,

(33) ═NOR¹²,

(34) ═NN(R¹²)C(O)N(R¹²)₂,

(35) —N(R¹²)C(O)N(R¹²)₂,

(36) —C(O)R¹²,

(37) —OC(O)R¹²,

(38) —N(R¹²)C(O)R¹²,

(39) —N(R²)C(O)OR¹²,

(40) —N(R¹²)S(O)ₙR¹²,

(41) —OR¹²,

(42) —S(O)ₙR¹²,

(43) —SC(O)R¹²,

(44) —OC(O)OR¹²,

(45) —N(R¹²)OR²,

(46) —OC(═N(R¹²))R¹²,

(47) —N(R¹²)C(═NR¹²)R¹²,

(48) —C(O)OC(O)R¹²,

(49) ═N—N(R¹²)—C(O)C(O)N(R¹²)₂,

(50) ═NN(R¹²)C(S)N(R¹²)₂,

(51) ═C(R¹²)OR¹²,

(52) alkylidene,

(53) optionally substituted spiroalkyl,

(54) optionally substituted spiroheterocycle, and

(55) ═N—N(R¹³)(R¹⁴),

wherein R¹² in (25)-(51) is independently selected from the group consisting of

(1) hydrogen,

(2) optionally substituted aryl,

(3) optionally substituted C₁-C₆ alkyl,

(4) optionally substituted C₃-C₆ alkenyl,

(5) optionally substituted aryl,

(6) optionally substituted arylalkyl, and

(7) optionally substituted heterocycle, and

wherein R¹³ and R¹⁴ in (55) together with the nitrogen atom to which they are attached form a heterocycle selected from the group consisting of pyrrolidinyl, piperidinyl, imidazolidinyl, pyrazolidinyl, piperazinyl, morpholinyl, and thiomorpholinyl,

wherein the heterocycle defined by R¹³ and R¹⁴ together can be optionally substituted with optionally substituted alkyl.

In another embodiment of the present invention are disclosed pharmaceutical compositions comprising the compounds in combination with a pharmaceutically acceptable carrier.

In yet another embodiment of the present invention are disclosed methods of inhibiting the growth of bacteria which comprises contacting the bacteria with an effective amount of the compounds.

In still yet another embodiment of the present invention is disclosed a method for preparing the compounds, the method comprising

(a) reacting compounds of formula (Ia)

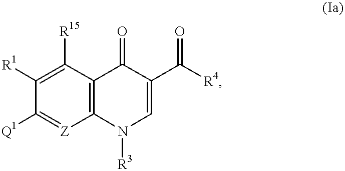

wherein R¹, R³, R⁴, R¹⁵, and Z are defined above, and Q¹ is a first covalent bond precursor, with compounds of formula (Ib)

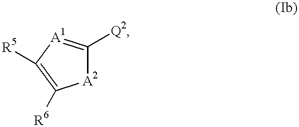

wherein A¹, A², and R⁵ and R⁶ are defined above, and Q² is a second covalent bond precursor, in the presence of a catalyst, to provide a first product; and

(b) optionally hydrolyzing the first product.

In another embodiment of the present invention are compounds of formula (Im)

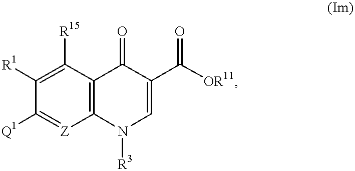

or pharmaceutically acceptable salts or prodrugs thereof, wherein

R^{1a} is optionally substituted alkyl;

R ¹⁵ is independently selected from

(1) hydrogen,

(2) optionally substituted alkyl,

(3) halide,

(4) nitro, and

(5) optionally protected amino;

Q¹ is selected from halide, methanesulfonate, and trifluoromethanesulfonate;

Z is nitrogen or

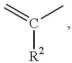

wherein R² is selected from

(1) hydrogen,

(2) optionally substituted alkyl,

(3) halide,

(4) optionally protected hydroxyl,

(5) —OR⁸, and

(6) —S(O)ₙR⁸, wherein n is zero, one, or two, and

wherein R⁸ in (4) and (5) is selected from

(a) C₃-C₆ alkenyl,

(b) C₁-C₆ alkyl, and

(c) C₃-C₆ alkynyl,

wherein (a)-(c) can be optionally substituted with one, two, or three substituents independently selected from

(i) alkoxy,

(ii) aryl,

(iii) C₃-C₆ cycloalkyl,

(iv) azido,

(v) cyano,

(vi) halide,

(vii) optionally protected amino,

(viii) optionally protected carboxyl, and

(ix) optionally protected hydroxyl;

R³ is selected from

(1) C₃-C₆ alkenyl,

(2) C₁-C₆ alkyl,

(3) C₃-C₆ alkynyl,

wherein (1)-(3) can be optionally substituted with one, two, or three substutuents independently selected from

(a) C₁-C₆ alkanoyloxy,

(b) C₁-C₆ alkoxy,

(c) aryl,

(d) azido,

(e) cyano,

(f) C₃-C₆ cycloalkyl,

(g) halide,

(h) optionally protected amino,

(i) optionally protected carboxyl,

(j) optionally protected hydroxyl,

(k) oxo,

(l) C₁-C₆ perfluoroalkoxy,

(m) C₁-C₆ perfluorothioalkoxy, and

(n) thioxo,

(4) aryl,

(5) C₃-C₆ cycloalkyl, and

(6) heterocycle,

wherein (4)-(6) can be optionally substituted with one, two, or three substituents independently selected from

(a) C₁-C₆ alkanoyloxy,

(b) C₁-C₆ alkoxy,

(c) C₂-C₆ alkenyl,

(d) C₁-C₆ alkyl,

(e) C₂-C₆ alkynyl,

(f) aryl,

(g) azido,

(h) cyano,

(i) C₃-C₆ cycloalkyl,

(j) halide,

(k) optionally protected amino

(l) optionally protected carboxyl,

(m) optionally protected hydroxyl,

(n) C₁-C₆ perfluoroalkoxy, and

(o) C₁-C₆ perfluorothioalkoxy; and

R¹¹ is hydrogen or a carboxyl protecting group.

In another embodiment of the compounds of formula (Im) of the present invention are compounds wherein R^{1a} is C₁-C₆ alkyl; and R³, R¹¹, R¹⁵, Q¹, and Z are as defined in formula (Im).

In a preferred embodiment of the compounds of formula (Im) of the present invention are compounds wherein R^{1a} is methyl; and R³, R¹¹, R¹⁵, Q¹, and Z are as defined in formula (Im).

In another embodiment of the compounds of formula (Im) of the present invention are compounds wherein R¹⁵ is hydrogen; and R^{1a}, R³, R¹¹, Q¹, and Z are as defined in formula (Im).

In another embodiment of the compounds of formula (Im) of the present invention are compounds wherein Q¹ is chloride; and R^{1a}, R³, R¹¹, R¹⁵, and Z are as defined in formula (Im).

In yet another embodiment of the compounds of formula (Im) of the present invention are compounds wherein Q¹ is bromide; and R^{1a}, R³, R¹¹, R¹⁵, and Z are as defined in formula (Im).

In another embodiment of the compounds of formula (Im) of the present invention are compounds wherein R³ is cyclopropyl; and R^{1a}, R¹¹, R¹⁵, Q¹, and Z are as defined in formula (Im).

In another embodiment of the compounds of formula (Im) of the present invention are compounds wherein Z is

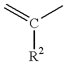

wherein R² is hydrogen; and R^{1a}, R³, R¹¹, R¹⁵, and Q¹ are as defined in formula (Im).

In another embodiment of the compounds of formula (Im) of the present invention are compounds wherein Z is nitrogen; and R^{1a}, R³, R¹¹, R¹⁵, and Q¹ are as defined in formula (Im).

Preferred compounds representative of this embodiment of the compounds of formula (Im) include, but are not limited to:

ethyl 7-bromo-1-cyclopropyl-6-methyl-4-oxo-1,4-dihydroquinoline-3-carboxylate; and

ethyl 7-chloro-1-cyclopropyl-6-methyl-4-oxo-1,4-dihydro-1,8-naphthyridine-3-carboxylate.

### DETAILED DESCRIPTION OF THE INVENTION

Definition of Terms

The term “alkanoyl” as used herein, refers to an alkyl group attached to the parent molecular group through a carbonyl group. The alkanoyl groups of this invention can be optionally substituted.

The term “alkanoyloxy,” as used herein, refers to an alkanoyl group attached to the parent molecular group through an oxygen atom. The alkanoyloxy groups of this invention can be optionally substituted.

The term “alkenyl,” as used herein, refers to a monovalent straight or branched chain hydrocarbon having from two to six carbon atoms and at least one carbon-carbon double bond. The alkenyl groups of this invention can be optionally substituted.

The term “alkenylene,” as used herein, refers to a divalent straight or branched chain hydrocarbon having from four to six carbons and at least one carbon-carbon double bond.

The term “alkoxy,” as used herein, refers to an alkyl group connected to the parent molecular group through an oxygen atom. The alkoxy groups of this invention can be optionally substituted.

The term “alkyl,” as used herein, refers to a monovalent straight or branched chain saturated hydrocarbon having from one to six carbon atoms. The alkyl groups of this invention can be optionally substituted.

The term “alkylidine,” as used herein, refers to ═CH₂.

The term “alkylene,” as used herein, refers to a divalent straight or branched chain saturated hydrocarbon having from one to six carbon atoms. The alkylene groups of this invention can be optionally substituted.

The term “alkynyl,” as used herein, refers to a monovalent straight or branched chain hydrocarbon having from two to six carbon atoms and at least one carbon-carbon triple bond. The alkynyl groups of this invention can be optionally substituted.

The term “alkynylene,” as used herein, refers to a monovalent straight or branched chain hydrocarbon having from four to six carbon atoms and at least one one carbon-carbon triple bond.

The terms “amino,” and “amino group,” as used herein, refer to —NH₂ or a derivative thereof formed by independent replacement of one or both hydrogen atoms thereon with one or two groups selected from optionally substituted C₁-C₆ alkyl, optionally substituted aryl, arylsulfonyl, heteroarylsulfonyl, aminosulfonyl, and heterocycle. The amino groupsof this invention can be optionally protected with amino protecting groups.

The term “amino protecting group,” as used herein, refers to selectively removable groups which protect amino groups against undesirable side reactions during synthetic procedures and includes all conventional amino protecting groups. Examples of amino protecting groups include optionally substituted acyl groups such as butoxycarbonyl, 4-chlorobutoxycarbonyl, ethoxycarbonyl, isobutoxycarbonyl, methoxycarbonyl, trichloroethoxycarbonyl, tribromoethoxycarbonyl, benzyloxycarbonyl, para-nitrobenzylcarbonyl, ortho-bromobenzyloxycarbonyl, chloroacetyl, dichloroacetyl, trichloroacetyl, trifluoroacetyl, phenylacetyl, formyl, acetyl, benzoyl, tert-amyloxycarbonyl, tert-butoxycarbonyl, para-methoxybenzyloxycarbonyl, 3,4-dimethoxybenzyloxycarbonyl, 4-(phenylazo)benzyloxycarbonyl, 2-furfaryloxycarbonyl, diphenylmethoxycarbonyl, 1,1-dimethylpropoxycarbonyl, isopropoxycarbonyl, phthaloyl, succinyl, glycyl, alanyl, D-alanyl, prolyl, D-prolyl, leucyl, D-leucyl, tyrosyl, N-methylleucyl, N-methyl-D-leucyl, norleucyl, D-norleucyl, D-tyrosyl, O-methyltyrosyl, O-methyl-D-tyrosyl, methionyl, D-methionyl, aspartyl, D-aspartyl, β-O-methylaspartyl, β-O-methyl-D-aspartyl, D-histidyl, histidyl, 1-adamantyloxycarbonyl, and 8-quinolyloxycarbonyl; optionally substituted arylalkyl groups such as benzyl, diphenylmethyl, and triphenylmethyl; optionally substituted arylthio groups such as 2-nitrophenylthio and 2,4-dinitrophenylthio; optionally substituted alkyl sulfonyl and optionally substituted arylsulfonyl groups such as methanesulfonyl, and para-toluenesulfonyl; optionally substituted dialkylaminoalkylidene groups such as N,N-dimethylaminomethylene; optionally substituted arylalkylidene groups such as benzylidene, 2-hydroxybenzylidene, 2-hydroxy-5-chlorobenzylidene, and 2-hydroxy-1-naphthylmethylene; optionally substituted nitrogen-containing heterocyclic alkylidene groups such as 3-hydroxy-4-pyridylmethylene; optionally substituted cycloalkylidene groups such as cyclohexylidene, 2-ethoxycarbonylcyclohexylidene, 2-ethoxycarbonylcyclopentylidene, 2-acetylcyclohexylidene, and 3,3-dimethyl-5-oxycyclohexylidene; optionally substituted diarylalkylphosphoryl and optionally substituted diarylalkylphosphoryl groups such as diphenylphosphoryl and dibenzylphosphoryl; optionally substituted oxygen-containing heterocyclic alkyl groups such as 5-methyl-2-oxo-2H-1,3-dioxol-4-yl-methyl; and optionally substituted silyl groups such as trimethylsilyl, triethylsilyl, and triphenylsilyl.

The term “aminosulfonyl,” as used herein, refers to an optionally protected amino group connected to the parent molecular group through a sulfonyl group.

The term “aryl,” as used herein, refers to phenyl, naphthyl, 1,2-dihydronaphthyl, and 1,2,3,4-tetrahydronaphthyl. The aryl groups of this invention can be optionally substituted.

The term “arylalkyl,” as used herein, refers to an aryl group connected to the parent molecular group through an alkyl group.

The term “arylsulfonyl,” as used herein, refers to an optionally substituted aryl group connected to the parent molecular group through a sulfonyl group.

The term “azido,” as used herein, refers to —N₃.

The term “carboxaldehyde,” as used herein, refers to —CHO.

The term “carbocyclic ring,” as used herein, refers to a non-aromatic five- to eight-membered hydrocarbon ring. The carbocyclic rings of this invention can be optionally substituted.

The term “carboxyl,” as used herein, refers to —CO₂H. The carboxyl groupsof this invention can be optionally protected with carboxyl protecting groups.

The term “cyano,” as used herein, refers to —CN.

The term “carboxyl protecting group,” as used herein, refers to selectively removable groups which protect hydroxyl groups against undesirable side reactions during synthetic procedures and includes all conventional carboxyl protecting groups. Examples of carboxyl protecting groups include optionally substituted alkyl groups such as methyl, ethyl, n-propyl, isopropyl, 1,1-dimethylpropyl, n-butyl, and tert-butyl; aryl groups such as phenyl, and naphthyl; optionally substituted arylalkyl groups such as benzyl, diphenylmethyl, triphenylmethyl, para-nitrobenzyl, para-methoxybenzyl, and bis(para-methoxyphenyl)methyl; optionally substituted acylalkyl groups such as acetylmethyl, benzoylmethyl, para-nitrobenzoylmethyl, para-bromobenzoylmethyl, and para-methanesulfonylbenzoylmethyl; optionally substituted oxygen-containing heterocyclic groups such as 2-tetrahydropyranyl and 2-tetrahydrofairanyl; optionally substituted haloalkyl groups such as 2,2,2-trichloroethyl; optionally substituted alkylsilylalkyl groups such as 2-(trimethylsilyl)ethyl; optionally substituted acyloxyalkyl groups such as acetoxymethyl, propionyloxymethyl, and pivaloyloxymethyl; optionally substituted nitrogen-containing heterocyclic groups such as phthalimidomethyl and succinimidomethyl; optionally substituted cycloalkyl groups such as cyclopropyl, cyclobutyl, cyclopentyl, and cyclohexyl; optionally substituted alkoxyalkyl groups such as methoxymethyl, methoxyethoxymethyl, and 2-(trimethylsilyl)ethoxymethyl; optionally substituted arylalkoxyalkyl groups such as benzyloxymethyl; optionally substituted alkylthioalkyl groups such as methylthiomethyl and 2-methylthioethyl; optionally substituted arylthioalkyl groups such as phenylthiomethyl; optionally substituted alkenyl groups such as

1,1-dimethyl-2-propenyl, 3-methyl-3-butenyl, and allyl; and optionally substituted silyl groups such as trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

The term “cycloalkyl,” as used herein, refers to a monovalent saturated cyclic or bicyclic hydrocarbon having three to ten carbon atoms. The cycloalkyl groups of this invention can be optionally substituted.

The term “first covalent bond precursor,” as used herein, refers to a leaving group or ligand. Examples of first covalent bond precursors include halide, methanesulfonate, and trifluoromethanesulfonate.

The terms “halo” or “halide” as used herein, refer to F, Cl, Br, or I.

The term “haloalkyl,” as used herein, refers to an alkyl group to which is attached at least one halide. The term “haloalkyl,” as used herein, also refers to perfluoroalkyl or perchloroalkyl.

The term “heteroarylsulfonyl,” as used herein, refers to an optionally substituted heterocycle connected to the parent molecular group through a sulfonyl group.

The term “heterocycle,” as used herein, refers to azetidinyl, benzofuranyl, benzothiazolyl, 2,1,3-benzoxadiazole, 2,1,3-benzothiadiazole, furyl, imidazolyl, imidazolinyl, imidazolidinyl, imidazo[2,1-b]thiazole, isoquinolinyl, isoxazolyl, isothiazolyl, oxazolyl, oxetanyl, morpholine, piperidinyl, pyrazinyl, pyrazolyl, pyridazinyl, pyridyl, pyrimidinyl, pyrrolidinyl, pyrrolyl, quinolinyl, tetrahydrofuranyl, thiazolyl, thienyl, thietanyl, thiomorpholine, thiomorpholine sulfone, or thiomorpholine sulfoxide. The heterocycle groups of this invention can be optionally substituted.

The term “heterocyclic ring,” as used herein, refers to a non-aromatic five- to eight-membered ring consisting of hydrocarbon groups and one or two groups selected from —NR¹⁰—, —O—, —S(O)ₙ—, or —NR¹⁰SO₂—. The heterocyclic rings of this invention can be optionally substituted.

The term “hydrogenation catalyst,” as used herein, refers to transition metals on a solid support or transition metal derivatives on a solid support.

The term “hydroxyl,” as used herein, refers to —OH. The hydroxyl groups of this invention can be optionally protected with hydroxyl protecting groups.

The term “hydroxyl protecting group,” as used herein, refers to selectively introducible and movable groups which protect hydroxyl groups against undesirable side reactions during synthetic procedures. Examples of hydroxyl protecting groups include optionally substituted acyl groups such as benzyloxycarbonyl, 4-nitrobenzyloxycarbonyl, 4-bromobenzyloxycarbonyl, 4-methoxybenzyloxycarbonyl, 3,4-dimethoxybenzyloxycarbonyl, methoxycarbonyl, ethoxycarbonyl, tert-butoxycarbonyl, 1,1-dimethylpropoxycarbonyl, isopropoxycarbonyl, isobutyloxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, 2-(triphenylphosphonio)ethoxycarbonyl, 2-furfuryloxycarbonyl, 1-adamantyloxycarbonyl, vinyloxycarbonyl, allyloxycarbonyl, S-benzylthiocarbonyl, 4-ethoxy-1-naphthyloxycarbonyl, 8-quinolyloxycarbonyl, acetyl, formyl, chloroacetyl, dichloroacetyl, trichloroacetyl, trifluoroacetyl, methoxyacetyl, phenoxyacetyl, pivaloyl, and benzoyl; optionally substituted alkyl groups such as methyl, tert-butyl, 2,2,2-trichloroethyl, and 2-trimethylsilylethyl; optionally substituted alkenyl groups such as as 1,1-dimethyl-2-propenyl, 3-methyl-3-butenyl, and allyl; optionally substituted arylalkyl groups such as benzyl, para-methoxybenzyl, 3,4-dimethoxybenzyl, diphenylmethyl, and triphenylmethyl; oxygen-containing and sulfur-containing heterocyclic groups such as tetrahydrofuryl, tetrahydropyranyl, and tetrahydrothiopyranyl; optionally substituted alkoxy and optionally substituted alkylthioalkyl groups such as methoxymethyl, methylthiomethyl, benzyloxymethyl, 2-methoxyethoxymethyl, 2,2,2-trichloroethoxymethyl, 2-(trimethylsilyl)ethoxymethyl, and 1-ethoxyethyl; alkylsulfonyl; optionally substituted arylsulfonyl groups such as methanesulfonyl, and para-toluenesulfonyl; and optionally substituted silyl groups such as trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

The term “nitro,” as used herein, refers to —NO₂.

The term “oxo,” as used herein, refers to a group formed by the replacement of two hydrogen atoms on the same carbon atom with a single oxygen atom.

The term “perchloroalkyl,” as used herein, refers to an alkyl group in which all of the hydrogen atoms have been replaced with chloride atoms.

The term “perfluoroalkoxy,” as used herein, refers to a perfluoroalkyl group attached to the parent molecular group through an oxygen atom.

The term “perfluoroalkyl,” as used herein, refers to an alkyl group in which all of the hydrogen atoms have been replaced with fluoride atoms.

The term “perfluorothioalkoxy,” as used herein, refers to a perfluoroalkyl group attached to the parent molecular group through a sulfur atom.

The term “pharmaceutically acceptable prodrugs,” as used herein, represents prodrugs of the compounds which are suitable for treatment of bacterial infections without undue toxicity, irritation, and allergic response, which are commensurate with a reasonable benefit/risk ratio, and which are effective for their intended use.

The term “prodrug,” as used herein, represents compounds which are rapidly transformed in vivo to the parent compounds by hydrolysis in blood. Prodrugs of the invention can include compounds wherein a nitrogen on the molecule has attached thereto an aminoacyl (1-mer), diaminoacyl (2-mer), or triaminoacyl (3-mer) group optionally capped with a carboxyl protecting group. The term “aminoacyl,” as used herein, refers to a group derived from naturally or unnaturally occuring amino acid in the racemic, D or L configuration. The terms “bisaminoacyl” and “trisaminoacyl,” as used herein, refer to di- and tri- aminoacyl groups, respectively. Other prodrugs of the invention include compounds wherein a carboxylic acid or amine group of the compounds is attached thereto a 2-oxo-1,3-dioxol-4-yl)methyl group such as reported in Chem. Pharm. Bull. 1985, 33(11), 4870-4877. Still other prodrugs of the invention include compounds wherein a tertiary amine group on the compounds has attached thereto a N-phosphonooxymethyl group such as reported in J. Med. Chem. 1999, 42(16), 3094-3100.

The term “pharmaceutically acceptable salt,” as used herein, represents salts or zwitterionic forms of the compounds which are water or oil-soluble or dispersible and are suitable for treatment of bacterial infections without undue toxicity, irritation, and allergic response, which are commensurate with a reasonable benefit/risk ratio, and which are effective for their intended use. The salts may be prepared during the final isolation and purification of the compounds or separately by reacting a free base group with a suitable acid. Representative acid addition salts include acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylpropionate, picrate, pivalate, propionate, succinate, tartrate, thiocyanate, trichloroacetic, trifluoroacetic, phosphate, glutamate, bicarbonate, para-toluenesulfonate, and undecanoate. Also, the basic nitrogen-containing groups can be quatemized with alkyl halides such as methyl, ethyl, propyl, and butyl chlorides, bromides and iodides; dialkyl sulfates such as dimethyl, diethyl, dibutyl, and diamyl sulfates; long chain halides such as decyl, lauryl, myristyl, and stearyl chlorides, bromides, and iodides; arylalkyl halides such as benzyl and phenethyl bromides. Examples of acids which may be employed to form pharmaceutically acceptable acid addition salts include inorganic acids such as hydrochloric, hydrobromic, sulphuric, and phosphoric and organic acids such as oxalic, maleic, succinic, and citric.

Basic addition salts can be prepared during the final isolation and purification of the compounds by reacting a carboxylic acid-containing group such as the one at the C-3 position of the quinoline or naphthyridine with a suitable base such as the hydroxide, carbonate, or bicarbonate of a metal cation or with ammonia or an organic primary, secondary or tertiary amine. Pharmaceutically acceptable salts include cations based on alkali metals or alkaline earth metals such as lithium, sodium, potassium, calcium, magnesium, and aluminum salts and nontoxic quaternary ammonia and amine cations such as ammonium, tetramethylammonium, tetraethylammonium, methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N-dibenzyl-1-phenethylamine, 1-ephenamine, and N,N′-dibenzylethylenediamine. Other representative organic amines useful for the formation of base addition salts include ethylenediamine, ethanolamine, diethanolamine, piperidine, and piperazine.

The term “protected amino,” as used herein, refers to an amino group derivatized by independent replacement of at least one hydrogen atom thereon by an amino protecting group.

The term “second covalent bond precursor,” as used herein, refers to a nucleophile or metal reagent, or a precursor thereof. Examples of second covalent bond precursors include trialkylstannane, boronic acid, boronic ester, magnesium halide, zinc halide, -silyl(alkyl)cyclobutane, and halide.

The term “spiroalkyl,” as used herein, refers to an alkylene group of three to six carbon atoms, both ends of which are bonded to the same carbon atom of the parent group.

The term “spiroheterocycle,” as used herein, refers to a heterocycle in which one of the ring carbon atoms is shared with one of the ring carbon atoms of the parent group. The spirohetereocycle groups of this invention can be optionally substituted.

The term “substituted alkanoyloxy,” as used herein, refers to an alkanoyloxy group derivatized by independent replacement of one, two, or three hydrogens thereon with a substituent or substituents independently selected from the group consisting of azido, cyano, halide, nitro, optionally protected amino, optionally protected hydroxyl, optionally protected carboxyl, optionally substituted C₁-C₆ alkoxy, optionally substituted aryl, optionally substituted C₃-C₆ cycloalkyl, optionally substituted heterocycle, oxo, C₁-C₆ perfluoroalkoxy, C₁-C₆ perfluorothioalkoxy, and thioxo.

The term “substituted alkenyl,” as used herein, refers to an alkenyl group derivatized by independent replacement of one, two, or three hydrogens thereon with a substituent or substituents independently selected from the group consisting of C₁-C₆ alkanoyloxy, C₁-C₆ alkoxy, aryl, azido, cyano, C₃-C₆ cycloalkyl, halide, optionally protected amino, optionally protected carboxyl, optionally protected hydroxyl, oxo, C₁-C₆ perfluoroalkoxy, C₁-C₆ perfluorothioalkoxy, and thioxo.

The term “substituted alkoxy,” as used herein, refers to an alkoxy group derivatized by independent replacement of one, two, or three hydrogens thereon with a substituent or substituents independently selected from the group consisting of C₁-C₆ alkanoyloxy, C₁-C₆ alkoxy, aryl, azido, cyano, C₃-C₆ cycloalkyl, halide, optionally protected amino, optionally protected carboxyl, optionally protected hydroxyl, oxo, C₁-C₆ perfluoroalkoxy, Cₗ-C₆ perfluorothioalkoxy, and thioxo.

The term “substituted alkyl,” as used herein, refers to an alkyl group derivatized by independent replacement of one, two, or three hydrogens thereon with a substituent or substituents independently selected from the group consisting of C₁-C₆ alkanoyloxy, C₁-C₆ alkoxy, aryl, heterocycle, azido, cyano, C₃-C₆ cycloalkyl, dimethylamino, halide, optionally protected amino, optionally protected carboxyl, optionally protected hydroxyl, oxo, C₁-C₆ perfluoroalkoxy, C₁-C₆ perfluorothioalkoxy, and thioxo.

The term “substituted alkynyl,” as used herein, refers to an alkynyl group derivatized by independent replacement of one, two, or three hydrogens thereon with a substituent or substituents independently selected from the group consisting of C₁-C₆ alkanoyloxy, C₁-C₆ alkoxy, aryl, azido, cyano, C₃-C₆ cycloalkyl, halide, optionally protected amino, optionally protected carboxyl, optionally protected hydroxyl, oxo, C₁-C₆ perfluoroalkoxy, Cₗ-C₆ perfluorothioalkoxy, and thioxo.

The term “substituted aryl,” as used herein, refers to an aryl group derivatized by independent replacement of one, two, or three hydrogens thereon with a substituent or substituents independently selected from the group consisting of C₁-C₆ alkanoyloxy, C₁-C₆ alkoxy, optionally substituted alkyl, aryl, azido, cyano, C₃-C₆ cycloalkyl, halide, optionally protected amino, optionally protected carboxyl, optionally protected hydroxyl, nitro, oxo, C₁-C₆ perfluoroalkoxy, C₁-C₆ perfluorothioalkoxy, and thioxo.

The term “substituted arylalkyl,” as used herein, refers to a substituted aryl group connected to the parent molecular group through an alkyl group.

The term “substituted carbocyclic ring,” as used herein, refers to a carbocyclic ring derivatized by independent replacement of one, two, three, or four hydrogens thereon with a substituent or substituents independently selected from the group consisting of C₁-C₆ alkanoyloxy, C₁-C₆ alkoxy, aryl, heterocycle, azido, cyano, C₃-C₆ cycloalkyl, halide, optionally substituted alkyl, optionally protected amino, optionally protected carboxyl, optionally protected hydroxyl, oxo, C₁-C₆ perfluoroalkoxy, C₁-C₆ perfluorothioalkoxy, and thioxo.

The term “substituted heterocyclic ring,” as used herein, refers to a heterocyclic ring derivatized by independent replacement of one, two, three, or four hydrogens thereon with a substituent or substituents independently selected from the group consisting of C₁-C₆ alkanoyloxy, C₁-C₆ alkoxy, aryl, heterocycle, azido, cyano, C₃-C₆ cycloalkyl, halide, optionally substituted alkyl, optionally protected amino, optionally protected carboxyl, optionally protected hydroxyl, oxo, C₁-C₆ perfluoroalkoxy, C₁-C₆ perfluorothioalkoxy, and thioxo.

The term “substituted cycloalkyl,” as used herein, refers to a cycloalkyl group derivatized by independent replacement of one, two, or three hydrogens thereon with a substituent or substituents independently selected from the group consisting of C₁-C₆ alkanoyloxy, C₁-C₆ alkoxy, aryl, azido, cyano, C₃-C₆ cycloalkyl, halide, optionally protected amino, optionally protected carboxyl, optionally protected hydroxyl, oxo, C₁-C₆ perfluoroalkoxy, C₁-C₆ perfluorothioalkoxy, and thioxo.

The term “substituted heterocycle,” as used herein, refers to a heterocycle derivatized by independent replacement of one, two, or three hydrogens thereon with a substituent or substituents independently selected from the group consisting of C₁-C₆ alkanoyloxy, C₁-C₆ alkoxy, optionally substituted alkyl, aryl, azido, cyano, C₃-C₆ cycloalkyl, halide, heterocycle, optionally protected amino, optionally protected carboxyl, optionally protected hydroxyl, oxo, C₁-C₆ perfluoroalkoxy, C₁-C₆ perfluorothioalkoxy, and thioxo.

The term “substituted spiroheterocycle,” as used herein, refers to a spiroheterocycle group derivatized by independent replacement of one, two, or three hydrogens thereon with a substituent or substituents independently selected from the group consisting of C₁-C₆ alkanoyloxy, C₁-C₆ alkoxy, aryl, heterocycle, azido, cyano, C₃-C₆ cycloalkyl, halide, optionally substituted alkyl, optionally protected amino, optionally protected carboxyl, optionally protected hydroxyl, oxo, C₁-C₆ perfluoroalkoxy, C₁-C₆ perfluorothioalkoxy, and thioxo.

The term “substituted thioalkoxy,” as used herein, refers to a thioalkoxy group derivatized by independent replacement of one, two, or three hydrogens thereon with a substituent or substituents independently selected from the group consisting of C₁-C₆ alkanoyloxy, C₁-C₆ alkoxy, aryl, azido, cyano, C₃-C₆ cycloalkyl, halide, optionally protected amino, optionally protected carboxyl, optionally protected hydroxyl, oxo, C₁-C₆ perfluoroalkoxy, C₁-C₆ perfluorothioalkoxy, and thioxo.

The term “sulfhydryl,” as used herein, refers to —SH.

The term “sulfonic,” as used herein, refers to —SO₃H.

The term “sulfonyl,” as used herein, refers to —SO₂—.

The term “thioalkoxy,” as used herein, refers to an alkyl group attached to the parent molecular group through a sulfur atom. The thioalkoxy groups of this invention can be optionally substituted.

The term “thiolcarboxyl,” as used herein, refers to —C(O)SH.

The term “thioxo,” as used herein, refers to a group formed by the replacement of two hydrogen atoms on the same carbon atom with a single sulfur atom.

It is intended that the definition of any substituent or variable at a particular part in a molecule be independent of its definition elsewhere in the molecule. Thus, for example, substituents such as —(CH₂)ₐC(O)R⁵ represent —CH₂C(O)H, and —CH₂C(O)CH₃; and substituents such as (CH₂)ₐN(R⁵)C(O)N(R⁵)₂ represent CH₂CH₂N(H)C(O)N(CH₃)(C₃H₇) and —CH₂N(CH₃)C(O)NH(CH₃), and the like.

Asymmetric centers can exist in the compounds of the invention. The compounds described herein may be stereoisomers or mixtures thereof. Individual stereoisomers of compounds are prepared by synthesis from starting materials containing the chiral centers or by preparation of mixtures of enantiomeric products followed by separation such as conversion to a mixture of diastereomers followed by separation or recrystallization, chromatographic techniques, or direct separation of the enantiomers on chiral chromatographic columns. Starting compounds of particular stereochemistry are either commercially available or are made by the methods described herein and resolved by techniques well-known in the art. It should be understood that the invention encompasses any stereoisomeric, diastereomeric, or enantiomeric form of the compounds of this invention, or mixtures thereof, which possess antibacterial activity, and is not limited to any one stereoisomer or stereoisomeric mixture.

According to the methods of treatment of the invention, the compounds can be administered alone, in combination with, or in concurrent therapy with other antibacterial agents. When using the compounds for antibacterial therapy, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally adminstered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, dilute acids or bases, dilute amino acid solutions, monoglycerides, diglycerides, fatty acids such as oleic acid, and fixed oils such as monoglycerides or diglycerides.

The antibacterial effect of parenterally administered compounds can be prolonged by slowing their absorption. One way to slow the absorption of a particular compound is adminstering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents; and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefor.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable nonirritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

A further possibility for delivery and utilization of the compounds is chemical conjugation of the compounds with other antibacterials. Similar dual-action conjugates are reported in U.S. Pat. No. 5,281,703. In the manner suggested by these references, a covalent bond can be formed between a functional group on a lactam and an amino group at the C-6 position or a carboxylic acid group at the C-3 position of the quinoline or naphthyridine.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Preferred embodiments for the practice of the invention include

Compounds of formula (I) wherein A¹ is methine,

Compounds of formula (I) wherein A² is —S—,

Compounds of formula (I) wherein R¹ is hydrogen,

Compounds of formula (I) wherein R¹ is fluoride,

Compounds of formula (I) wherein R¹⁵ is hydrogen,

Compounds of formula (I) wherein Z is

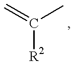

wherein R² is OR⁸ wherein R⁸ is methyl or difluoromethyl,

Compounds of formula (I) wherein R³ is cyclopropyl,

Compounds of formula (I) wherein R⁵ and R⁶ together are an optionally substituted carbocyclic ring,

Compounds of formula (I) wherein R⁵ and R⁶ together are an optionally substituted carbocyclic ring and the substituent is amino, and

Compounds of formula (I) wherein R⁵ and R⁶ together are an optionally substituted heterocyclic ring.

Accordingly, taking the listing of preferred substituents and combinations thereof, it will be appreciated by one skilled in the art that compounds of formula (I) wherein A¹ is methine, A² is —S—, R¹ is hydrogen, Z is

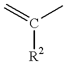

wherein R² is OR⁸ wherein R⁸ is methyl, R³ is cyclopropyl, R⁴ is OR¹¹ wherein R¹¹ is hydrogen, R¹⁵ is hydrogen, R⁵ and R⁶ together are an optionally substituted carbocyclic ring and the substituent is amino, are contemplated as being within the scope of the present invention.

Specific compounds of the invention include

1-cyclopropyl-4-oxo-7-(4,5,6,7-tetrahydrothieno[3,2-c]pyridin-2-yl)-1,4-dihydro-3-quinolinecarboxylic acid,

1-cyclopropyl-8-methoxy-4-oxo-7-(4,5,6,7-tetrahydrothieno[3,2-c]pyridin-2-yl)-1,4-dihydro-3-quinolinecarboxylic acid,

1-cyclopropyl-4-oxo-7-(4,5,6,7-tetrahydrothieno[3,2-c]pyridin-2-yl)-1,4-dihydro[1,8]naphthyridine-3-carboxylic acid,

1-cyclopropyl-6-fluoro-4-oxo-7-(4,5,6,7-tetrahydrothieno[3,2-c]pyridin-2-yl)-1,4-dihydro[1,8]naphthyridine-3-carboxylic acid,

1-cyclopropyl-8-(difluoromethoxy)-4-oxo-7-(4,5,6,7-tetrahydrothieno[3,2-c]pyridin-2-yl)-1,4-dihydro-3-quinolinecarboxylic acid,

1-cyclopropyl-4-oxo-7-(4,5,6,7-tetrahydrothieno[2,3-c]pyridin-2-yl)-1,4-dihydro-3-quinolinecarboxylic acid,

1-cyclopropyl-8-methoxy-4-oxo-7-(4,5,6,7-tetrahydrothieno[2,3-c]pyridin-2-yl)-1,4-dihydro-3-quinolinecarboxylic acid,

1-cyclopropyl-4-oxo-7-(4,5,6,7-tetrahydrothieno[2,3-c]pyridin-2-yl)-1,4-dihydro[1,8]naphthyridine-3-carboxylic acid,

1-cyclopropyl-6-fluoro-4-oxo-7-(4,5,6,7-tetrahydrothieno[2,3-c]pyridin-2-yl)-1,4-dihydro[1,8]naphthyridine-3-carboxylic acid,

1-cyclopropyl-8-(difluoromethoxy)-4-oxo-7-(4,5,6,7-tetrahydrothieno[2,3 -c]pyridin-2-yl)-1,4-dihydro-3-quinolinecarboxylic acid,

1-cyclopropyl-7-(6-methyl-4,5,6,7-tetrahydrothieno[3,2-c]pyridin-2-yl)-4-oxo-1,4-dihydro[1,8]naphthyridine-3-carboxylic acid,

1-cyclopropyl-6-fluoro-7-(6-methyl-4,5,6,7-tetrahydrothieno[3,2-c]pyridin-2-yl)-4-oxo-1,4-dihydro[1,8]naphthyridine-3-carboxylic acid,

1-cyclopropyl-7-(6-methyl-4,5,6,7-tetrahydrothieno[3,2-c]pyridin-2-yl)-4-oxo-1,4-dihydro-3-quinolinecarboxylic acid,

1-cyclopropyl-8-(difluoromethoxy)-7-(6-methyl-4,5,6,7-tetrahydrothieno[3,2-c]pyridin-2-yl)-4-oxo-1,4-dihydro-3-quinolinecarboxylic acid,

1-cyclopropyl-8-methoxy-7-(6-methyl-4,5,6,7-tetrahydrothieno[3,2-c]pyridin-2-yl)-4-oxo-1,4-dihydro-3-quinolinecarboxylic acid,

1-cyclopropyl-7-(4-methyl-4,5,6,7-tetrahydrothieno[3,2-c]pyridin-2-yl)-4-oxo-1,4-dihydro-3-quinolinecarboxylic acid,

1-cyclopropyl-7-(4-methyl-4,5,6,7-tetrahydrothieno[3,2-c]pyridin-2-yl)-4-oxo-1,4-dihydro[1,8]naphthyridine-3-carboxylic acid,

1-cyclopropyl-6-fluoro-7-(4-methyl-4,5,6,7-tetrahydrothieno[3,2-c]pyridin-2-yl)-4-oxo-1,4-dihydro[1,8]naphthyridine-3-carboxylic acid,

1-cyclopropyl-8-methoxy-7-(4-methyl-4,5,6,7-tetrahydrothieno[3,2-c]pyridin-2-yl)-4-oxo-1,4-dihydro-3-quinolinecarboxylic acid,

1-cyclopropyl-8-(difluoromethoxy)-7-(4-methyl-4,5,6,7-tetrahydrothieno[3,2-c]pyridin-2-yl)-4-oxo-1,4-dihydro-3-quinolinecarboxylic acid,

1-cyclopropyl-7-(7-methyl-4,5,6,7-tetrahydrothieno[2,3-c]pyridin-2-yl)-4-oxo-1,4-dihydro[1,8]naphthyridine-3-carboxylic acid,

1-cyclopropyl-6-fluoro-7-(7-methyl-4,5,6,7-tetrahydrothieno[2,3-c]pyridin-2-yl)-4-oxo-1,4-dihydro[1,8]naphthyridine-3-carboxylic acid,

1-cyclopropyl-8-methoxy-7-(7-methyl-4,5 ,6,7-tetrahydrotheino[2,3-c]pyrindin-2-yl)-4-oxo-1,4-dihydro-3-quinolinecarboxylic acid,

1-cyclopropyl-7-(7,7-dimethyl-4,5,6,7-tetrahydrothieno[3,2-c]pyridin-2-yl)-4-oxo-1,4-dihydro[1,8]naphthyridine-3-carboxylic acid,

1-cyclopropyl-7-(7,7-dimethyl-4,5,6,7-tetrahydrothieno[3,2-c]pyridin-2-yl)-4-oxo-1,4-dihydro-3-quinolinecarboxylic acid,

1-cyclopropyl-7-(7,7-dimethyl-4,5,6,7-tetrahydrothieno[3,2-c]pyridin-2-yl)-8-methoxy-4-oxo-1,4-dihydro-3-quinolinecarboxylic acid,

1-cyclopropyl-7-(7,7-dimethyl-4,5,6,7-tetrahydrothieno[3,2-c]pyridin-2-yl)-6-fluoro-4-oxo-1,4-dihydro[1,8]naphthyridine-3-carboxylic acid,

1-cyclopropyl-8-(difluoromethoxy)-7-(7,7-dimethyl-4,5,6,7-tetrahydrothieno[3,2-c]pyridin-2-yl)-4-oxo-1,4-dihydro-3-quinolinecarboxylic acid,

1-cyclopropyl-7-(4-methyl-4,5,6,7-tetrahydrothieno[2,3-c]pyridin-2-yl)-4-oxo-1,4-dihydro-3-quinolinecarboxylic acid,

1-cyclopropyl-8-methoxy-7-(4-methyl-4,5,6,7-tetrahydrothieno[2,3-c]pyridin-2-yl)-4-oxo-1,4-dihydro-3-quinolinecarboxylic acid,

1-cyclopropyl-8-(difluoromethoxy)-7-(4-methyl-4,5,6,7-tetrahydrothieno[2,3-c]pyridin-2-yl)-4-oxo-1,4-dihydro-3-quinolinecarboxylic acid,

1-cyclopropyl-7-(4-methyl-4,5,6,7-tetrahydrothieno[2,3-c]pyridin-2-yl)-4-oxo-1,4-dihydro[1,8]naphthyridine-3-carboxylic acid,

1-cyclopropyl-6-fluoro-7-(4-methyl-4,5 ,6,7-tetrahydrothieno[2,3-c]pyridin-2-yl)-4-oxo-1,4-dihydro[1,8]naphthyridine-3-carboxylic acid,

1-cyclopropyl-7-(4,4-dimethyl-4,5,6,7-tetrahydrothieno[2,3-c]pyridin-2-yl)-8-methoxy-4-oxo-1,4-dihydro-3-quinolinecarboxylic acid,

1-cyclopropyl-6-fluoro-7-(4-hydroxy-4,5,6,7-tetrahydro-1-benzothien-2-yl)-4-oxo-1,4-dihydro[1,8]naphthyridine-3-carboxylic acid,

1-cyclopropyl-7-(4-hydroxy-5,6-dihydro-4H-thieno[2,3-b]thiopyran-2-yl)-8-methoxy-4-oxo-1,4-dihydro-3-quinolinecarboxylic acid,

7-(4-amino-5,6-dihydro-4H-thieno[2,3-b]thiopyran-2-yl)-1-cyclopropyl-8-methoxy-4-oxo-1,4-dihydro-3-quinolinecarboxylic acid,

1-cyclopropyl-7-(4-hydroxy-4,5,6,7-tetrahydro-1-benzothien-2-yl)-8-methoxy-4-oxo-1,4-dihydro-3-quinolinecarboxylic acid,

7-(4-azido-4,5,6,7-tetrahydro-1-benzothien-2-yl)-1-cyclopropyl-8-methoxy-4-oxo-1,4-dihydro-3-quinolinecarboxylic acid,

7-(4-amino-4,5,6,7-tetrahydro-1-benzothien-2-yl)-1-cyclopropyl-8-methoxy-4-oxo-1,4-dihydro-3-quinolinecarboxylic acid,

1-cyclopropyl-7-(7-hydroxy-4,5,6,7-tetrahydro-1-benzothien-2-yl)-8-methoxy-4-oxo-1,4-dihydro-3-quinolinecarboxylic acid,

1-cyclopropyl-7-(5-hydroxy-4,5,6,7-tetrahydro-1-benzothien-2-yl)-8-methoxy-4-oxo-1,4-dihydro-3-quinolinecarboxylic acid,

1-cyclopropyl-7-(5-(hydroxymethyl)-4,5,6,7-tetrahydro-1-benzothien-2-yl)-8-methoxy-4-oxo-1,4-dihydro-3-quinolinecarboxylic acid,

1-cyclopropyl-7-(5-(hydroxymethyl)-6,7-dihydro-1-benzothien-2-yl)-8-methoxy-4-oxo-1,4-dihydro-3-quinolinecarboxylic acid,

1-cyclopropyl-7-(5-hydroxy-5,6-dihydro-4H-cyclopenta[b]thien-2-yl)-8-methoxy-4-oxo-1,4-dihydro-3-quinolinecarboxylic acid,

1-cyclopropyl-8-methoxy-7-(4-methoxy-5,6-dihydro-4H-cyclopenta[b]thien-2-yl)-4-oxo-1,4-dihydro-3-quinolinecarboxylic acid,

1-cyclopropyl-7-(6-hydroxy-4,5,6,7-tetrahydro-1-benzothien-2-yl)-8-methoxy-4-oxo-1,4-dihydro-3-quinolinecarboxylic acid,

1-cyclopropyl-6-fluoro-4-oxo-7-(4-oxo-4,5,6,7-tetrahydro-1-benzothien-2-yl)-1,4-dihydro[1,8]naphthyridine-3-carboxylic acid,

1-cyclopropyl-6-fluoro-7-((4E/Z)-4-(hydroxyimino)-4,5,6,7-tetrahydro-1-benzothien-2-yl)-4-oxo- 1 ,4-dihydro( 1 ,8)naphthyridine-3-carboxylic acid;

1-cyclopropyl-8-methoxy-4-oxo-7-(4-oxo-4,5,6,7-tetrahydro-1-benzothien-2-yl)- 1,4-dihydro-3-quinolinecarboxylic acid;

1-cyclopropyl-7-((4E/Z)-4-(hydroxyimino)-4,5,6,7-tetrahydro-1-benzothien-2-yl)-8-methoxy-4-oxo-1,4-dihydro-3-quinolinecarboxylic acid;

1-cyclopropyl-8-diflouromethoxy-7-(7-methyl-4,5,6,7-tetrahydrotheino(2,3-c)pyrindin-2-yl)-4-oxo- 1,4-dihydro-3 -quinolinecarboxylic acid;

1-cyclopropyl-8-methoxy-4-oxo-7-(5-oxo-4,5,6,7-tetrahydro-1-benzothien-2-yl)-1,4-dihydro-3-quinolinecarboxylic acid;

7-[5-(azidomethyl)-4,5,6,7-tetrahydro-1-benzothien-2-yl]-1-cyclopropyl-8-methoxy-4-oxo-1,4-dihydro-3-quinolinecarboxylic acid;

1-cyclopropyl-7-((5E/Z)-5-(hydroxyimino)-4,5,6,7-tetrahydro-1-benzothien-2-yl)-8-methoxy-4-oxo-1,4-dihydro-3-quinolinecarboxylic acid;

1-cyclopropyl-8-methoxy-7-((5E/Z)-5-(methoxyimino)-4,5,6,7-tetrahydro-1-benzothien-2-yl)-4-oxo-1,4-dihydro-3-quinolinecarboxylic acid;

1-cyclopropyl-7-(5-methyl-4,5,6,7-tetrahydrothieno[2,3-c]pyridin-2-yl)-4-oxo-1,4-dihydro[1,8]naphthyridine-3-carboxylic acid;

7-(5-bromo-4-oxo-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(6-oxo-4,5,6,7-tetrahydro-1-benzothien-2-yl)-1,4-dihydro-3-quinolinecarboxylic acid;

1-cyclopropyl-7-((6E/Z)-6-(hydroxyimino)-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(7-oxo-4,5,6,7-tetrahydro-1-benzothien-2-yl)-1,4-dihydro-3-quinolinecarboxylic acid;

7-(5-azido-4-oxo-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-((7E/Z)-7-(methoxyimino)-4,5,6,7-tetrahydro-1-benzothien-2-yl)-4-oxo-1,4-dihydro-3-quinolinecarboxylic acid;

1-cyclopropyl-8-methoxy-7-((4E/Z)-4-(methoxyimino)-4,5,6,7-tetrahydro-1-benzothien-2-yl)-4-oxo-1,4-dihydro-3-quinolinecarboxylic acid;

1-cyclopropyl-7-((4E/Z)-4-(ethoxyimino)-4,5,6,7-tetrahydro-1-benzothien-2-yl)-8-methoxy-4-oxo-1,4-dihydro-3-quinolinecarboxylic acid;

1-cyclopropyl-8-methoxy-7-((6E/Z)-6-(methoxyimino)-4,5,6,7-tetrahydro-1-benzothien-2-yl)-4-oxo-1,4-dihydro-3-quinolinecarboxylic acid;

1-cyclopropyl-7-(6,7-dihydro-1-benzothien-2-yl)-8-methoxy-4-oxo-1,4-dihydro-3-quinolinecarboxylic acid;

1-cyclopropyl-8-methoxy-7-((4E/Z)-4-(4-morpholinylimino)-4,5,6,7-tetrahydro-1-benzothien-2-yl)-4-oxo-1,4-dihydro-3-quinolinecarboxylic acid;

1-cyclopropyl-7-(4,5-dihydroxy-4,5,6,7-tetrahydro-1-benzothien-2-yl)-8-methoxy-4-oxo-1,4-dihydro-3-quinolinecarboxylic acid;

7-(5-(aminomethyl)-4,5,6,7-tetrahydro-1-benzothien-2-yl)-1-cyclopropyl-8-methoxy-4-oxo-1,4-dihydro-3-quinolinecarboxylic acid;

7-(6-amino-4,5,6,7-tetrahydro-1-benzothien-2-yl)-1-cyclopropyl-8-methoxy-4-oxo-1,4-dihydro-3-quinolinecarboxylic acid;

7-((4E/Z)-4-(tert-butoxyimino)-4,5,6,7-tetrahydro-1-benzothien-2-yl)-1-cyclopropyl-8-methoxy-4-oxo-1,4-dihydro-3-quinolinecarboxylic acid;

7-((4E/Z)-4-((benzyloxy)imino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-((4E/Z)-4-(1-pyrrolidinylimino)-4,5,6,7-tetrahydro-1-benzothien-2-yl)-1,4-dihydro-3-quinolinecarboxylic acid;

7-(4-amino-4,5,6,7-tetrahydro-1-benzothien-3-yl)-1-cyclopropyl-8-methoxy-4-oxo-1,4-dihydro-3-quinolinecarboxylic acid;

7-(5-amino-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-[4-(methylamino)-4,5,6,7-tetrahydro-1-benzothien-2-yl]-4-oxo-1,4-dihydro-3-quinolinecarboxylic acid;

1-cyclopropyl-7-((5E/Z)-5-(ethoxyimino)-4,5,6,7-tetrahydro-1-benzothien-2-yl)-8-methoxy-4-oxo-1,4-dihydro-3-quinolinecarboxylic acid;

7-((5E/Z)-5-((benzyloxy)imino)-4,5,6,7-tetrahydro-1-benzothien-2-yl)-1-cyclopropyl-8-methoxy-4-oxo-1,4-dihydro-3-quinolinecarboxylic acid;

7-((4E/Z)-4-((aminocarbonyl)hydrazono)-4,5,6,7-tetrahydro-1-benzothien-2-yl)1-cyclopropyl-8-methoxy-4-oxo-1,4-dihydro-3-quinolinecarboxylic acid;

ethyl 1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylate;

ethyl 1-cyclopropyl-8-methoxy-7-((4E/Z)-4-(((2R)-2-(methoxymethyl)pyrrolidinyl)imino)-4,5,6,7-tetrahydro-1-benzothien-2-yl)-4-oxo-1,4-dihydro-3-quinolinecarboxylate;

1-cyclopropyl-7-(4-(dimethylamino)-4,5,6,7-tetrahydro-1-benzothien-2-yl)-8-methoxy-4-oxo-1,4-dihydro-3-quinolinecarboxylic acid;

7-((4E/Z)-4-[(aminocarbothioyl)hydrazono)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-((4E/Z)-4-(((methylamino)carbothioyl)hydrazono)-4,5,6,7-tetrahydro-1-benzothien-2-yl)-4-oxo-1,4-dihydro-3-quinolinecarboxylic acid;

1-cyclopropyl-8-methoxy-7-(5-methylene-4-oxo-4,5,6,7-tetrahydro-1-benzothien-2-yl)-4-oxo-1,4-dihydro-3-quinolinecarboxylic acid;

ethyl 1-cyclopropyl-8-methoxy-7-(5-methylene-4-oxo-4,5,6,7-tetrahydro-1-benzothien-2-yl)-4-oxo-1,4-dihydro-3-quinolinecarboxylate;

1-cyclopropyl-8-methoxy-7-(4-((methylsulfonyl)amino)-4,5,6,7-tetrahydro-1-benzothien-2-yl)-4-oxo-1,4-dihydro-3-quinolinecarboxylic acid;

1-cyclopropyl-8-methoxy-4-oxo-7-(4-(1H-pyrrol-1-yl)-4,5,6,7-tetrahydro-1-benzothien-2-yl)-1,4-dihydro-3-quinolinecarboxylic acid;

1-cyclopropyl-7-((4E/Z)-4-(((ethylamino)carbothioyl)hydrazono)-4,5,6,7-tetrahydro-1-benzothien-2-yl)-8-methoxy-4-oxo-1,4-dihydro-3-quinolinecarboxylic acid;

7-((4E/Z)-4-((amino(oxo)acetyl)hydrazono)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylic acid;

7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-((3-pyridinylmethyl)amino)-4,5,6,7-tetrahydro-1-benzothien-2-yl)-1,4-dihydro-3-quinolinecarboxylic acid;

ethyl 7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylate;

7-((4E/Z)-4-(acetylhydrazono)-4,5,6,7-tetrahydro-1-benzothien-2-yl)-1-cyclopropyl-8-methoxy-4-oxo-1,4-dihydro-3-quinolinecarboxylic acid;

7-(4-(benzylamino)-4,5,6,7-tetrahydro-1-benzothien-2-yl)-1-cyclopropyl-8-methoxy-4-oxo- 1,4-dihydro-3-quinolinecarboxylic acid;

1-cyclopropyl-7-(4-(ethylamino)-4,5,6,7-tetrahydro-1-benzothien-2-yl)-8-methoxy-4-oxo-1,4-dihydro-3-quinolinecarboxylic acid;

1-cyclopropyl-8-(difluoromethoxy)-7-(4-hydroxy-4,5,6,7-tetrahydro-1-benzothien-2-yl)-4-oxo-1,4-dihydro-3-quinolinecarboxylic acid;

1-cyclopropyl-7-((4E/Z)-4-(4,5-dihydro-1H-imidazol-2-ylhydrazono)-4,5,6,7-tetrahydro-1-benzothien-2-yl)-8-methoxy-4-oxo-1,4-dihydro-3-quinolinecarboxylic acid;

7-(4-amino-4,5,6,7-tetrahydro-1-benzothien-2-yl)-1-cyclopropyl-8-(difluoromethoxy)-4-oxo-1,4-dihydro-3-quinolinecarboxylic acid;

1-cyclopropyl-7-(4-hydroxy-2-methyl-1,1-dioxido-3,4-dihydro-2H-thieno[3,2-e][1,2]thiazin-6-yl)-8-methoxy-4-oxo-1,4-dihydro-3-quinolinecarboxylic acid;

ethyl 1-cyclopropyl-8-methoxy-7-(5-methyl-4-oxo-4,5,6,7-tetrahydro-1-benzothien-2-yl)-4-oxo-1,4-dihydro-3-quinolinecarboxylate;

1-cyclopropyl-7-(4-((3-fluorobenzyl)amino)-4,5,6,7-tetrahydro-1-benzothien-2-yl)-8-methoxy-4-oxo-1,4-dihydro-3-quinolinecarboxylic acid;

7-((4E/Z)-4-[(aminocarbothioyl)(methyl)hydrazono)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-(5 -methyl-4-oxo-4,5,6,7-tetrahydro- 1 -benzothien-2-yl)-4-oxo-1,4-dihydro-3-quinolinecarboxylic acid;

1-cyclopropyl-8-(difluoromethoxy)-7-(4-(methylamino)-4,5,6,7-tetrahydro-1-benzothien-2-yl)-4-oxo-1,4-dihydro-3-quinolinecarboxylic acid;

1-cyclopropyl-6-fluoro-7-(4-(methylamino)-4,5,6,7-tetrahydro-1-benzothien-2-yl)-4-oxo-1,4-dihydro[1,8]naphthyridine-3-carboxylic acid;

7-(4-amino-2-methyl-1,1-dioxido-3,4-dihydro-2H-thieno[3,2-e][1,2]thiazin-6-yl)-1-cyclopropyl-8-methoxy-4-oxo-1,4-dihydro-3-quinolinecarboxylic acid;

1-cyclopropyl-7-(4-(hydroxymethyl)-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(4-(1-pyrrolidinylmethyl)-4,5,6,7-tetrahydro-1-benzothien-2-yl)-1,4-dihydro-3-quinolinecarboxylic acid;

1-cyclopropyl-8-methoxy-4-oxo-7-(4-((2-pyrrolidinylmethyl)amino)-4,5,6,7-tetrahydro-1-benzothien-2-yl)-1,4-dihydro-3-quinolinecarboxylic acid;

7-(4-(acetylamino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-(propionylamino)-4,5,6,7-tetrahydro-1-benzothien-2-yl)-1,4-dihydro-3-quinolinecarboxylic acid;

1-cyclopropyl-8-methoxy-7-(4-((methoxyacetyl)amino)-4,5,6,7-tetrahydro-1-benzothien-2-yl)-4-oxo-1,4-dihydro-3-quinolinecarboxylic acid;

1-cyclopropyl-8-methoxy-4-oxo-7-(4-((tetrahydrofuranyl-2-carbonyl)amino)-4,5,6,7-tetrahydro-1-benzothien-2-yl)-1,4-dihydro-3-quinolinecarboxylic acid;

1-cyclopropyl-8-methoxy-4-oxo-7-(4-((tetrahydroffiranyl-3-carbonyl)amino)-4,5,6,7-tetrahydro-l-benzothien-2-yl)-1,4-dihydro-3-quinolinecarboxylic acid;

1-cyclopropyl-8-methoxy-7-(4-((4-morpholinylacetyl)amino)-4,5,6,7-tetrahydro-1-benzothien-2-yl)-4-oxo-1,4-dihydro-3-quinolinecarboxylic acid;

1-cyclopropyl-8-methoxy-7-(4-((3-(4-morpholinyl)propanoyl)amino)-4,5,6,7-tetrahydro-1-benzothien-2-yl)-4-oxo-1,4-dihydro-3-quinolinecarboxylic acid;

1-cyclopropyl-8-methoxy-4-oxo-7-(4-((1H-pyrrol-2-ylcarbonyl)amino)-4,5,6,7-tetrahydro-1-benzothien-2-yl)-1,4-dihydro-3-quinolinecarboxylic acid;

1-cyclopropyl-8-methoxy-4-oxo-7-(4-((3-pyridinylacetyl)amino)-4,5,6,7-tetrahydro-1-benzothien-2-yl)-1,4-dihydro-3-quinolinecarboxylic acid;

1-cyclopropyl-8-methoxy-4-oxo-7-(4-((3-pyridazinylcarbonyl)amino)-4,5,6,7-tetrahydro-1-benzothien-2-yl)-1,4-dihydro-3-quinolinecarboxylic acid;

1-cyclopropyl-7-(4-((1H-imidazol-2-ylcarbonyl)amino)-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(4-((1,3-thiazol-2-ylcarbonyl)amino)-4,5,6,7-tetrahydro- 1 -benzothien-2-yl)-1,4-dihydro-3-quinolinecarboxylic acid;

1-cyclopropyl-8-methoxy-7-(5-(methylamino)-4,5,6,7-tetrahydro-1-benzothien-2-yl)-4-oxo-1,4-dihydro-3-quinolinecarboxylic acid;

1-cyclopropyl-8-methoxy-7-(4-(4-morpholinylmethyl)-4,5,6,7-tetrahydro-1-benzothien-2-yl)-4-oxo-1,4-dihydro-3-quinolinecarboxylic acid;

1-cyclopropyl-7-(4-((dimethylamino)methyl)-4,5,6,7-tetrahydro-1-benzothien-2-yl)-8-methoxy-4-oxo-1,4-dihydro-3-quinolinecarboxylic acid;

1-cyclopropyl-7-(4-(((dimethylamino)acetyl)amino)-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(4-((2-pyridinylacetyl)amino)-4,5,6,7-tetrahydro-1-benzothien-2-yl)-1,4-dihydro-3-quinolinecarboxylic acid;

7-(4-(aminomethyl)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-((4E/Z)-4-((4-pyridinylmethoxy)imino)-4,5,6,7-tetrahydro-1-benzothien-2-yl)-1,4-dihydro-3-quinolinecarboxylic acid;

7-(4-((2-aminoethyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-(4-((1-methyl-⁴-piperidinyl)amino)-4,5,6,7-tetrahydro-1-benzothien-2-yl)-4-oxo-1,4-dihydro-3-quinolinecarboxylic acid;

1-cyclopropyl-7-((4E/Z)-4-(hydroxyimino)-5-methyl-4,5,6,7-tetrahydro-1-benzothien-2-yl)-8-methoxy-4-oxo-1,4-dihydro-3-quinolinecarboxylic acid;

1-cyclopropyl-7-(4-hydroxy-7,7-dioxido-5,6-dihydro-4H-thieno[2,3-b]thiopyran-2-yl)-8-methoxy-4-oxo-1,4-dihydro-3-quinolinecarboxylic acid;

7-(4-(((5-chloro-1,3-dimethyl-1H-pyrazol-4-yl)sulfonyl)amino)-4,5,6,7-tetrahydro-1-benzothien-2-yl)-1-cyclopropyl-8-methoxy-4-oxo-1,4-dihydro-3-quinolinecarboxylic acid;

7-(4-(((4-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-((phenylsulfonyl)amino)-4,5,6,7-tetrahydro-1-benzothien-2-yl)-1,4-dihydro-3-quinolinecarboxylic acid;

7-(4-(((2-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-(4-(((4-methoxyphenyl)sulfonyl)amino)-4,5,6,7-tetrahydro-1-benzothien-2-yl)-4-oxo-1,4-dihydro-3-quinolinecarboxylic acid;

1-cyclopropyl-8-methoxy-7-(4-(((3-nitrophenyl)sulfonyl)amino)-4,5,6,7-tetrahydro-1-benzothien-2-yl)-4-oxo-1,4-dihydro-3-quinolinecarboxylic acid;

1-cyclopropyl-8-methoxy-4-oxo-7-(5-((2-pyrrolidinylmethyl)amino)-4,5,6,7-tetrahydro-1-benzothien-2-yl)-1,4-dihydro-3-quinolinecarboxylic acid;

7-(4-amino-7,7-dioxido-5,6-dihydro-4H-thieno[2,3-b]thiopyran-2-yl)-1-cyclopropyl-8-methoxy-4-oxo-1,4-dihydro-3-quinolinecarboxylic acid;

1-cyclopropyl-7-(4-(((3,5-dimethyl-4-isoxazolyl)sulfonyl)amino)-4,5,6,7-tetrahydro-1-benzothien-2-yl)-8-methoxy-4-oxo-1,4-dihydro-3-quinolinecarboxylic acid;

7-(4-((2,1,3-benzoxadiazol-4-ylsulfonyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-7-(4-(((dimethylamino)sulfonyl)amino)-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(4-((2-thienylsulfonyl)amino)-4,5,6,7-tetrahydro-1-benzothien-2-yl)-1,4-dihydro-3-quinolinecarboxylic acid;

7-(4-(((3-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;

7-(4-(((4-(acetylamino)phenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;

7-(4-((2,1,3-benzothiadiazol-4-ylsulfonyl)amino)-4,5,6,7-tetrahydro-1 -benzothien-2-yl)-1-cyclopropyl-8-methoxy-4-oxo-1,4-dihydro-3-quinolinecarboxylic acid;

1-cyclopropyl-7-(4-(((5-(3-isoxazolyl)-2-thienyl)sulfonyl)amino)-4,5,6,7-tetrahydro-1-benzothien-2-yl)-8-methoxy-4-oxo-1,4-dihydro-3-quinolinecarboxylic acid;

1-cyclopropyl-7-(4-(((4-fluorophenyl)sulfonyl)amino)-4,5,6,7-tetrahydro-1-benzothien-2-yl)-8-methoxy-4-oxo-1,4-dihydro-3-quinolinecarboxylic acid;

7-(4-(((6-chloroimidazo[2,1-b][1,3]thiazol-5-yl)sulfonyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-((4-pyridinylacetyl)amino)-4,5,6,7-tetrahydro-1-benzothien-2-yl)-1,4-dihydro-3-quinolinecarboxylic acid;

1-cyclopropyl-7-(4-((2-hydroxyethyl)amino)-4,5,6,7-tetrahydro-1-benzothien-2-yl)-8-methoxy-4-oxo-1,4-dihydro-3-quinolinecarboxylic acid;

7-(4-amino-4,5,6,7-tetrahydro-1-benzothien-2-yl)-1-cyclopropyl-6-fluoro-4-oxo-1,4-dihydro[1,8]naphthyridine-3-carboxylic acid;

7-(4-((glycyl)amino)-4,5,6,7-tetrahydro-1-benzothien-2-yl)-1-cyclopropyl-8-methoxy-4-oxo-1,4-dihydro-3-quinolinecarboxylic acid;

7-(4-((D-alanyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-((D-prolyl)amino)-4,5,6,7-tetrahydro-1-benzothien-2-yl)-1,4-dihydro-3-quinolinecarboxylic acid;

7-(4-(((2R)-2-amino-3-(1H-imidazol-5-yl)propanoyl)amino)-4,5,6,7-tetrahydro-1-benzothien-2-yl)-1-cyclopropyl-8-methoxy-4-oxo- 1,4-dihydro-3-quinolinecarboxylic acid;

7-(4-((glcyl)amino)-4,5,6,7-tetrahydro-1-benzothien-2-yl)-1-cyclopropyl-8-methoxy-4-oxo-1,4-dihydro-3-quinolinecarboxylic acid;

7-(4-((D-tyrosyl)amino)-4,5,6,7-tetrahydro-1-benzothien-2-yl)-1-cyclopropyl-8-methoxy-4-oxo-1,4-dihydro-3-quinolinecarboxylic acid;

7-(4-((O-methyl-D-tyrosyl)amino)-4,5,6,7-tetrahydro-1-benzothien-2-yl)-1-cyclopropyl-8-methoxy-4-oxo-1,4-dihydro-3-quinolinecarboxylic acid;

7-(4-((D-methionyl)amino)-4,5,6,7-tetrahydro- 1 -benzothien-2-yl)-1-cyclopropyl-8-methoxy-4-oxo-1,4-dihydro-3-quinolinecarboxylic acid;

7-(4-(((2R)-2-amino-3-(3-pyridinyl)propanoyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-(((2R)-piperidinylcarbonyl)amino)-4,5,6,7-tetrahydro-1-benzothien-2-yl)-1,4-dihydro-3-quinolinecarboxylic acid;

1-cyclopropyl-8-methoxy-4-oxo-7-(4-((4-pyrimidinylcarbonyl)amino)-4,5,6,7-tetrahydro-1-benzothien-2-yl)-1,4-dihydro-3-quinolinecarboxylic acid;

1-cyclopropyl-8-methoxy-4-oxo-7-(4-((1,3-thiazol-2-ylcarbonyl)amino)-4,5,6,7-tetrahydro-1-benzothien-2-yl)-1,4-dihydro-3-quinolinecarboxylic acid;

1-cyclopropyl-8-methoxy-4-oxo-7-(4-((phenylacetyl)amino)-4,5,6,7-tetrahydro-1-benzothien-2-yl)-1,4-dihydro-3-quinolinecarboxylic acid;

1-cyclopropyl-7-(4-(3-furoylamino)-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(4-((2-pyridinylcarbonyl)amino)-4,5,6,7-tetrahydro-1-benzothien-2-yl)-1,4-dihydro-3-quinolinecarboxylic acid;

1-cyclopropyl-8-methoxy-4-oxo-7-(4-((1H-pyrazol-4-ylcarbonyl)amino)-4,5,6,7-tetrahydro-1-benzothien-2-yl)-1,4-dihydro-3-quinolinecarboxylic acid;

7-(4-((D-aspartyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-7-((4R)-4-((N-methyl-D-leucyl)amino)-4,5,6,7-tetrahydro-1-benzothien-2-yl)-4-oxo-1,4-dihydro-3-quinolinecarboxylic acid;

7-((4R)-4-((D-norleucyl)amino)-4,5,6,7-tetrahydro-1-benzothien-2-yl)-1-cyclopropyl-8-methoxy-4-oxo-1,4-dihydro-3-quinolinecarboxylic acid;

methyl 1-cyclopropyl-8-methoxy-4-oxo-7-(4-((4-pyrimidinylcarbonyl)amino)-4,5,6,7-tetrahydro-1-benzothien-2-yl)-1,4-dihydro-3-quinolinecarboxylate;

methyl 1-cyclopropyl-8-methoxy-4-oxo-7-(4-((1,3-thiazol-2-ylcarbonyl)amino)-4,5,6,7-tetrahydro-1-benzothien-2-yl)-1,4-dihydro-3-quinolinecarboxylate;

methyl 1-cyclopropyl-8-methoxy-4-oxo-7-(4-((2-pyridinylcarbonyl)amino)-4,5,6,7-tetrahydro-1-benzothien-2-yl)-1,4-dihydro-3-quinolinecarboxylate;

7-(4-((β-O-methyl-D-aspartyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-((4E)-4-((3-pyridinylmethoxy)imino)-4,5,6,7-tetrahydro-1-benzothien-2-yl)-1,4-dihydro-3-quinolinecarboxylic acid;

1-cyclopropyl-8-methoxy-4-oxo-7-((4E)-4-((2-pyridinylmethoxy)imino)-4,5,6,7-tetrahydro- 1 -benzothien-2-yl)-1,4-dihydro-3-quinolinecarboxylic acid;

ethyl 7-(4-((tert-butyl(dimethyl)silyl)oxy)-5-methyl-4,5,6,7-tetrahydro-1-benzothien-2-yl)- 1 -cyclopropyl-8-methoxy-4-oxo-1,4-dihydro-3-quinolinecarboxylate;

1-cyclopropyl-7-(4-hydroxy-5-methyl-4,5,6,7-tetrahydro-1-benzothien-2-yl)-8-methoxy-4-oxo-1,4-dihydro-3-quinolinecarboxylic acid;

1-cyclopropyl-8-methoxy-7-(4-(((methylanilino)carbonyl)amino)-4,5,6,7-tetrahydro-1-benzothien-2-yl)-4-oxo-1,4-dihydro-3-quinolinecarboxylic acid;

1-cyclopropyl-7-(4-(((diethylamino)carbonyl)amino)-4,5,6,7-tetrahydro-1-benzothien-2-yl)-8-methoxy-4-oxo-1,4-dihydro-3-quinolinecarboxylic acid;

1-cyclopropyl-7-(4-(((diisopropylamino)carbonyl)amino)-4,5 ,6,7-tetrahydro-1-benzothien-2-yl)-8-methoxy-4-oxo-1,4-dihydro-3-quinolinecarboxylic acid;

1-cyclopropyl-8-methoxy-7-(4-((4-morpholinylcarbonyl)amino)-4,5,6,7-tetrahydro-1-benzothien-2-yl)-4-oxo-1,4-dihydro-3-quinolinecarboxylic acid;

1-cyclopropyl-8-methoxy-7-(4-((methoxycarbonyl)amino)-4,5,6,7-tetrahydro-1-benzothien-2-yl)-4-oxo-1,4-dihydro-3-quinolinecarboxylic acid;

7-(4-(((benzyloxy)carbonyl)amino)-4,5,6,7-tetrahydro- 1-benzothien-2-yl)-1-cyclopropyl-8-methoxy-4-oxo-1,4-dihydro-3-quinolinecarboxylic acid;

1-cyclopropyl-7-(4-((isobutoxycarbonyl)amino)-4,5,6,7-tetrahydro-1-benzothien-2-yl)-8-methoxy-4-oxo-1,4-dihydro-3-quinolinecarboxylic acid;

1-cyclopropyl-7-(4-((ethoxycarbonyl)amino)-4,5,6,7-tetrahydro-1-benzothien-2-yl)-8-methoxy-4-oxo-1,4-dihydro-3-quinolinecarboxylic acid;

7-(4-((butoxycarbonyl)amino)-4,5,6,7-tetrahydro-1-benzothien-2-yl)-1-cyclopropyl-8-methoxy-4-oxo-1,4-dihydro-3-quinolinecarboxylic acid;

7-(4-(((4-chlorobutoxy)carbonyl)amino)-4,5,6,7-tetrahydro-1-benzothien-2-yl)-1-cyclopropyl-8-methoxy-4-oxo-1,4-dihydro-3-quinolinecarboxylic acid;

1-cyclopropyl-7-((5E/Z)-5-(hydroxymethylene)-4-oxo-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-((4E/Z)-4-((3-pyridinylmethoxy)imino)-4,5,6,7-tetrahydro-1-benzothien-2-yl)-1,4-dihydro-3-quinolinecarboxylic acid;

1-cyclopropyl-7-(4-(3-hydroxy-1-azetidinyl)-4,5,6,7-tetrahydro-1-benzothien-2-yl)-8-methoxy-4-oxo-1,4-dihydro-3-quinolinecarboxylic acid;

7-(4-amino-5-methyl-4,5,6,7-tetrahydro-1-benzothien-2-yl)-1-cyclopropyl-8-methoxy-4-oxo-1 ,4-dihydro-3-quinolinecarboxylic acid;

7-(4-amino-4,5,6,7-tetrahydro-1-benzothien-2-yl)-1-cyclopropyl-4-oxo-1,4-dihydro[1,8]naphthyridine-3-carboxylic acid;

7-(7-amino-4,5,6,7-tetrahydro-1-benzothien-2-yl)-1-cyclopropyl-8-methoxy-4-oxo-1,4-dihydro-3-quinolinecarboxylic acid;

1-cyclopropyl-7-(7-hydroxy-4,5,6,7-tetrahydrothieno[3,2-c]pyridin-2-yl)-8-methoxy-4-oxo-1,4-dihydro-3-quinolinecarboxylic acid;

1-cyclopropyl-8-methoxy-4-oxo-7-(7-oxo-4,5,6,7-tetrahydrothieno[3,2-c]pyridin-2-yl)-1,4-dihydro-3-quinolinecarboxylic acid;

1-cyclopropyl-7-(5,5-difluoro-4-hydroxy-4,5,6,7-tetrahydro- 1 -benzothien-2-yl)-8-methoxy-4-oxo-1,4-dihydro-3-quinolinecarboxylic acid;

ethyl 7-(4-azido-5-methyl-4,5,6,7-tetrahydro-1-benzothien-2-yl)-1-cyclopropyl-8-methoxy-4-oxo-1,4-dihydro-3-quinolinecarboxylate;

7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid;

1-cyclopropyl-7-(5-(hydroxymethyl)-5-methyl-4-oxo-4,5,6,7-tetrahydro-1-benzothien-2-yl)-8-methoxy-4-oxo-1,4-dihydro-3-quinolinecarboxylic acid;

7-(7-amino-4,5,6,7-tetrahydrothieno[3,2-c]pyridin-2-yl)-1-cyclopropyl-8-methoxy-4-oxo-1,4-dihydro-3-quinolinecarboxylic acid;

1-cyclopropyl-7-(4-hydroxy-4,5,6,7-tetrahydrothieno[2,3-c]pyridin-2-yl)-8-methoxy-4-oxo-1,4-dihydro-3-quinolinecarboxylic acid;

1-cyclopropyl-6-fluoro-8-methoxy-7-(4-(methylamino)-4,5,6,7-tetrahydro-1-benzothien-2-yl)-4-oxo-1,4-dihydro-3-quinolinecarboxylic acid hydrochloride;

1-cyclopropyl-8-methoxy-4-oxo-7-(4-oxo-4,5,6,7-tetrahydrothieno[2,3-c]pyridin-2-yl)-1,4-dihydro-3-quinolinecarboxylic acid;

7-[4-(3-amino-1-azetidinyl)-4,5,6,7-tetrahydro-1-benzothien-2-yl]-1-cyclopropyl-8-methoxy-4-oxo-1,4-dihydro-3-quinolinecarboxylic acid;

1-cyclopropyl-7-(4-hydroxy-5,5-dimethyl-4,5,6,7-tetrahydro-1-benzothien-2-yl)-8-methoxy-4-oxo-1,4-dihydro-3-quinolinecarboxylic acid;

7-(4-((tert-butoxycarbonyl)amino)-5-methyl-4,5,6,7-tetrahydro-1-benzothien-2-yl)-1-cyclopropyl-8-methoxy-4-oxo-1,4-dihydro-3-quinolinecarboxylic acid;

methyl 7-[4-(acetyloxy)-4,5,6,7-tetrahydro-1-benzothien-2-yl]-1-cyclopropyl-8-methoxy-4-oxo-1,4-dihydro-3-quinolinecarboxylate;

1-cyclopropyl-8-methoxy-7-(7-(methylamino)-4,5,6,7-tetrahydro-1-benzothien-2-yl)-4-oxo-1,4-dihydro-3-quinolinecarboxylic acid;

methyl 1-cyclopropyl-6-fluoro-7-(4-hydroxy-4,5,6,7-tetrahydro-1-benzothien-2-yl)-8-methoxy-4-oxo-1,4-dihydro-3-quinolinecarboxylate;

1-cyclopropyl-7-(7-hydroxy-4,4-dimethyl-4,5,6,7-tetrahydro-1-benzothien-2-yl)-8-methoxy-4-oxo-1,4-dihydro-3-quinolinecarboxylic acid;

methyl 7-(4-azido-4,5,6,7-tetrahydro-1-benzothien-2-yl)-1-cyclopropyl-6-fluoro-8-methoxy-4-oxo-1,4-dihydro-3-quinolinecarboxylate;

methyl 7-(4-((tert-butoxycarbonyl)amino)-4,5,6,7-tetrahydro-1-benzothien-2-yl)-1-cyclopropyl-6-fluoro-8-methoxy-4-oxo-1,4-dihydro-3-quinolinecarboxylate;

7-(4-((tert-butoxycarbonyl)amino)-4,5,6,7-tetrahydro-1-benzothien-2-yl)-1-cyclopropyl-6-fluoro-8-methoxy-4-oxo-1,4-dihydro-3-quinolinecarboxylic acid;

7-(7-amino-4,4-dimethyl-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(4-oxo-5-spiro-3′-(N-benzylpyrrolidine)-4,5,6,7-tetrahydro-1-benzothien-2-yl)-1,4-dihydro-3-quinolinecarboxylic acid;

7-(4-amino-4,5,6,7-tetrahydro-1-benzothien-2-yl)-1-cyclopropyl-6-fluoro-8-methoxy-4-oxo-1,4-dihydro-3-quinolinecarboxylic acid;

7-(4-amino-4,5,6,7-tetrahydrothieno[2,3-c]pyridin-2-yl)-1-cyclopropyl-8-methoxy-4-oxo-1,4-dihydro-3-quinolinecarboxylic acid;

7-(7-azido-4,5,6,7-tetrahydrothieno[3,2-c]pyridin-2-yl)-1-cyclopropyl-8-methoxy-4-oxo-1,4-dihydro-3-quinolinecarboxylic acid;

1-cyclopropyl-7-(4,4-dimethyl-4,5,6,7-tetrahydro-1-benzothien-2-yl)-8-methoxy-4-oxo-1,4-dihydro-3-quinolinecarboxylic acid;

7-(7-amino-4,5,6,7-tetrahydro-1-benzothien-2-yl)-1-cyclopropyl-6-fluoro-8-methoxy-4-oxo-1,4-dihydro-3-quinolinecarboxylic acid;

7-(5-acetyl-7-amino-4,5,6,7-tetrahydrothieno[3,2-c]pyridin-2-yl)-1-cyclopropyl-8-methoxy-4-oxo-1,4-dihydro-3-quinolinecarboxylic acid;

7-(7-amino-5-(methylsulfonyl)-4,5,6,7-tetrahydrothieno [3,2-c]pyridin-2-yl)-1-cyclopropyl-8-methoxy-4-oxo-1,4-dihydro-3-quinolinecarboxylic acid;

7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid;

7-(4-amino-3-methyl-4,5,6,7-tetrahydro-1-benzothien-2-yl)-1-cyclopropyl-8-methoxy-4-oxo-1,4-dihydro-3-quinolinecarboxylic acid;

1-cyclopropyl-7-((4E/Z)-4-(hydroxyimino)-5,5-dimethyl-4,5,6,7-tetrahydro-1-benzothien-2-yl)-8-methoxy-4-oxo-1,4-dihydro-3-quinolinecarboxylic acid;

1-cyclopropyl-7-(4-hydroxy-6,6-dimethyl-4,5,6,7-tetrahydro-1-benzothien-2-yl)-8-methoxy-4-oxo-1,4-dihydro-3-quinolinecarboxylic acid;

7-(7-amino-6-methyl-4,5,6,7-tetrahydro-1-benzothien-2-yl)-1-cyclopropyl-8-methoxy-4-oxo-1,4-dihydro-3-quinolinecarboxylic acid hydrochloride;

1-cyclopropyl-7-(6,6-dimethyl-4,5,6,7-tetrahydro-1-benzothien-2-yl)-8-methoxy-4-oxo-1,4-dihydro-3-quinolinecarboxylic acid;

7-(7-amino-6-fluoro-4,5,6,7-tetrahydro-1-benzothien-2-yl)-1-cyclopropyl-8-methoxy-4-oxo-1,4-dihydro-3-quinolinecarboxylic acid;

7-(4-amino-6,6-dimethyl-4,5,6,7-tetrahydro-1-benzothien-2-yl)-1-cyclopropyl-8-methoxy-4-oxo-1,4-dihydro-3-quinolinecarboxylic acid;

1-cyclopropyl-8-methoxy-4-oxo-7-(7-oxo-4,5 ,6,7-tetrahydro-1,3-benzothiazol-2-yl)-1,4-dihydro-3-quinolinecarboxylic acid;

1-cyclopropyl-7-(6-fluoro-7-hydroxy-4,5,6,7-tetrahydro-1-benzothien-2-yl)-8-methoxy-4-oxo-1,4-dihydro-3-quinolinecarboxylic acid;

1-cyclopropyl-7-(7-hydroxy-6,6-dimethyl-4,5,6,7-tetrahydro-1-benzothien-2-yl)-8-methoxy-4-oxo-1,4-dihydro-3-quinolinecarboxylic acid;

1-cyclopropyl-7-((6S,7S)-7-hydroxy-6-methyl-4,5,6,7-tetrahydro-1-benzothien-2-yl)-8-methoxy-4-oxo-1,4-dihydro-3-quinolinecarboxylic acid;

1-cyclopropyl-8-methoxy-4-oxo-7-(4,5,6,7-tetrahydro-1-benzothien-2-yl)-1,4-dihydro-3-quinolinecarboxylic acid;

7-(7-amino-6,6-difluoro-4,5,6,7-tetrahydro-1-benzothien-2-yl)-1-cyclopropyl-8-methoxy-4-oxo-1,4-dihydro-3-quinolinecarboxylic acid;

7-(7-amino-6,6-dimethyl-4,5,6,7-tetrahydro-1-benzothien-2-yl)-1-cyclopropyl-8-methoxy-4-oxo-1,4-dihydro-3-quinolinecarboxylic acid;

1-cyclopropyl-6-fluoro-8-methoxy-7-(7-(methylamino)-4,5,6,7-tetrahyro-1-benzothien-2-yl)-4-oxo-1,4-dihydro-3-quinolinecarboxylic acid;

1-cyclopropyl-8-methoxy-4-oxo-7-(5-oxo-5,6-dihydro-4H-cyclopenta[b]thien-2-yl)-1,4-dihydro-3-quinolinecarboxylic acid;

1-cyclopropyl-8-methoxy-7-((5E/Z)-5-(methoxyimino)-5,6-dihydro-4H-cyclopenta[b]thien-2-yl)-4-oxo-1,4-dihydro-3-quinolinecarboxylic acid;

7-(5-amino-5,6-dihydro-4H-cyclopenta[b]thien-2-yl)-1-cyclopropyl-8-methoxy-4-oxo-1,4-dihydro-3-quinolinecarboxylic acid;

1-cyclopropyl-7-(5-((ethoxycarbonyl)amino)-5,6-dihydro-4H-cyclopenta[b]thien-2-yl)-8-methoxy-4-oxo-1,4-dihydro-3-quinolinecarboxylic acid;

1-cyclopropyl-8-methoxy-7-(5-((methoxycarbonyl)amino)-5,6-dihydro-4H-cyclopenta[b]thien-2-yl)-4-oxo-1,4-dihydro-3-quinolinecarboxylic acid;

7-(5-(acetylamino)-5,6-dihydro-4H-cyclopenta[b]thien-2-yl)-1-cyclopropyl-8-methoxy-4-oxo-1,4-dihydro-3-quinolinecarboxylic acid;

1-cyclopropyl-8-methoxy-7-(5-(((4-methylphenyl)sulfonyl)amino)-5,6-dihydro-4H-cyclopenta[b]thien-2-yl)-4-oxo-1,4-dihydro-3-quinolinecarboxylic acid;

1-cyclopropyl-8-methoxy-7-(5-((methylsulfonyl)amino)-5,6-dihydro-4H-cyclopenta[b]thien-2-yl)-4-oxo-1,4-dihydro-3-quinolinecarboxylic acid;

7-((5E/Z)-5-((benzyloxy)imino)-5,6-dihydro-4H-cyclopenta[b]thien-2-yl)-1-cyclopropyl-8-methoxy-4-oxo-1,4-dihydro-3-quinolinecarboxylic acid;

1-cyclopropyl-7-((5E/Z)-5-(hydroxyimino)-5,6-dihydro-4H-cyclopenta[b]thien-2-yl)-8-methoxy-4-oxo-1,4-dihydro-3-quinolinecarboxylic acid;

7-(4-amino-4-methyl-4,5,6,7-tetrahydro-1-benzothien-2-yl)-1-cyclopropyl-8-methoxy-4-oxo-1,4-dihydro-3-quinolinecarboxylic acid;

1-cyclopropyl-7-(5-(dimethylamino)-5,6-dihydro-4H-cyclopenta[b]thien-2-yl)-8-methoxy-4-oxo-1,4-dihydro-3-quinolinecarboxylic acid;

7-(4-azido-5,5-dimethyl-4,5,6,7-tetrahydro-1-benzothien-2-yl)-1-cyclopropyl-8-methoxy-4-oxo-1,4-dihydro-3-quinolinecarboxylic acid;

7-(7-amino-4,5,6,7-tetrahydro-1-benzothien-3-yl)-1-cyclopropyl-8-methoxy-4-oxo-1,4-dihydro-3-quinolinecarboxylic acid;

7-(7-amino-4,5,6,7-tetrahydro-1-benzothien-2-yl)-1-cyclopropyl-8-(difluoromethoxy)-4-oxo-1,4-dihydro-3-quinolinecarboxylic acid;

1-cyclopropyl-7-(7-hydroxy-5,5-dimethyl-4,5,6,7-tetrahydro-1-benzothien-2-yl)-8-methoxy-4-oxo-1,4-dihydro-3-quinolinecarboxylic acid;

7-(7-amino-5,5-dimethyl-4,5,6,7-tetrahydro-1-benzothien-2-yl)-1-cyclopropyl-8-methoxy-4-oxo-1,4-dihydro-3-quinolinecarboxylic acid;

1-cyclopropyl-7-(4-hydroxy-4,5,6,7-tetrahydro-1-benzofuran-2-yl)-8-methoxy-4-oxo-1,4-dihydro-3-quinolinecarboxylic acid;

and

7-(4-amino-4,5,6,7-tetrahydro-1-benzofuran-2-yl)-1-cyclopropyl-8-methoxy-4-oxo-1,4-dihydro-3-quinolinecarboxylic acid;

7-(7-hydroxy-6-spirocyclohexyl-4,5,6,7-tetrahydro-1-benzothien-2-yl)-1-cyclopropyl-8-methoxy-4-oxo-1,4-dihydroquinoline-3-carboxylic acid;

7-(7-amino-5,5-dimethyl-4,5,6,7-tetrahydro-1-benzothien-2-yl)-1-cyclopropyl-8-methoxy-4-oxo-1,4-dihydroquinoline-3-carboxylic acid;

7-(6-(2-aminoethyl)-4,5,6,7-tetrahydro-1-benzothien-2-yl)-1-cyclopropyl-8-methoxy-4-oxo-1,4-dihydroquinoline-3-carboxylic acid;

7-(7-amino-4,5,6,7-tetrahydro-1-benzothien-2-yl)-1-cyclopropyl-6-fluoro-4-oxo-1,4-dihydro-1,8-naphthyridine-3-carboxylic acid;

1-(2,4-difluorophenyl)-6-fluoro-7-(7-hydroxy-4,5,6,7-tetrahydro- 1-benzothien-2-yl)-4-oxo-1,4-dihydro-1,8-naphthyridine-3-carboxylic acid;

7-(4-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid;

1-(2,4-difluorophenyl)-6-fluoro-7-(4-hydroxy-4,5,6,7-tetrahydro-1-benzothien-2-yl)-4-oxo-1,4-dihydro-1,8-naphthyridine-3-carboxylic acid;

1-(2,4-difluorophenyl)-6-fluoro-7-(4-(methylamino)-4,5,6,7-tetrahydro-1-benzothien-2-yl)-4-oxo-1,4-dihydro-1,8-naphthyridine-3-carboxylic acid;

7-(7-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid;

1-(2,4-difluorophenyl)-6-fluoro-8-methoxy-7-(4-(methylamino)-4,5,6,7-tetrahydro-1-benzothien-2-yl)-4-oxo-1,4-dihydroquinoline-3-carboxylic acid;

1-cyclopropyl-7-(4-(methylamino)-4,5,6,7-tetrahydro-1-benzothien-2-yl)-4-oxo-1,4-dihydroquinoline-3-carboxylic acid;

1-cyclopropyl-7-(7-hydroxy-4,5,6,7-tetrahydro-1-benzothien-2-yl)-4-oxo-1,4-dihydroquinoline-3-carboxylic acid;

1-cyclopropyl-7-(4-hydroxy-4,5,6,7-tetrahydro-1-benzothien-2-yl)-4-oxo-1,4-dihydroquinoline-3-carboxylic acid;

7-(7-amino-4,5,6,7-tetrahydro-1-benzothien-2-yl)-1-cyclopropyl-4-oxo-1,4-dihydro-1,8-naphthyridine-3-carboxylic acid;

1-(2,4-difluorophenyl)-6-fluoro-7-(4-hydroxy-4,5,6,7-tetrahydro-1-benzothien-2-yl)-8-methoxy-4-oxo-1,4-dihydroquinoline-3-carboxylic acid;

1-cyclopropyl-5-fluoro-7-(4-(methylamino)-4,5,6,7-tetrahydro-1-benzothien-2-yl)-4-oxo-1,4-dihydroquinoline-3-carboxylic acid;

1-(2,4-difluorophenyl)-6-fluoro-4-oxo-7-(4,5,6,7-tetrahydro-1-benzothien-2-yl)-1,4-dihydro-1,8-naphthyridine-3-carboxylic acid;

7-(4-amino-4,5,6,7-tetrahydro-1-benzothien-2-yl)-1-cyclopropyl-4-oxo-1,4-dihydroquinoline-3-carboxylic acid;

7-(7-amino-4,5,6,7-tetrahydro-1-benzothien-2-yl)-1-cyclopropyl-4-oxo-1,4-dihydroquinoline-3-carboxylic acid;

1-cyclopropyl-5,8-difluoro-7-(4-(methylamino)-4,5,6,7-tetrahydro-1-benzothien-2-yl)-4-oxo-1,4-dihydroquinoline-3 -carboxylic acid;

1-cyclopropyl-6-fluoro-7-(4-(methylamino)-4,5,6,7-tetrahydro-1-benzothien-2-yl)-4-oxo-1,4-dihydroquinoline-3-carboxylic acid;

1-cyclopropyl-6,8-difluoro-7-(4-(methylamino)-4,5,6,7-tetrahydro-1-benzothien-2-yl)-4-oxo-1,4-dihydroquinoline-3-carboxylic acid;

1-(2,4-difluorophenyl)-7-(7-hydroxy-4,5,6,7-tetrahydro-1-benzothien-2-yl)-8-methoxy-4-oxo-1,4-dihydroquinoline-3-carboxylic acid;

1-cyclopropyl-6-fluoro-7-(7-hydroxy-4,5,6,7-tetrahydro-1-benzothien-2-yl)-4-oxo-1,4-dihydroquinoline-3-carboxylic acid;

1-cyclopropyl-6,8-difluoro-7-(4-hydroxy-4,5,6,7-tetrahydro-1-benzothien-2-yl)-4-oxo-1,4-dihydroquinoline-3-carboxylic acid;

1-cyclopropyl-6,8-difluoro-7-(7-hydroxy-4,5,6,7-tetrahydro-1-benzothien-2-yl)-4-oxo-1,4-dihydroquinoline-3-carboxylic acid;

1-cyclopropyl-6-fluoro-7-(4-hydroxy-4,5,6,7-tetrahydro-1-benzothien-2-yl)-4-oxo-1,4-dihydroquinoline-3-carboxylic acid;

1-(2,4-difluorophenyl)-7-(4-hydroxy-4,5,6,7-tetrahydro-1-benzothien-2-yl)-8-methoxy-4-oxo-1,4-dihydroquinoline-3-carboxylic acid;

1-(2,4-difluorophenyl)-6-fluoro-7-(7-hydroxy-4,5,6,7-tetrahydro-1-benzothien-2-yl)-8-methoxy-4-oxo-1,4-dihydroquinoline-3-carboxylic acid;

1-cyclopropyl-6-fluoro-7-(7-hydroxy-4,5,6,7-tetrahydro-1-benzothien-2-yl)-8-methoxy-4-oxo-1,4-dihydroquinoline-3-carboxylic acid;

1-cyclopropyl-5,8-difluoro-7-(4-hydroxy-4,5,6,7-tetrahydro-1-benzothien-2-yl)-4-oxo-1,4-dihydroquinoline-3-carboxylic acid;

1-cyclopropyl-5,8-difluoro-7-(7-hydroxy-4,5,6,7-tetrahydro-1-benzothien-2-yl)-4-oxo-1,4-dihydroquinoline-3-carboxylic acid;

1-cyclopropyl-7-(7-((4-fluorobenzyl)amino)-4,5,6,7-tetrahydro-1-benzothien-2-yl)-8-methoxy-4-oxo-1,4-dihydroquinoline-3-carboxylic acid;

1-cyclopropyl-5-fluoro-7-(4-hydroxy-4,5,6,7-tetrahydro-1-benzothien-2-yl)-4-oxo-1,4-dihydroquinoline-3-carboxylic acid;

1-(2,4-difluorophenyl)-8-methoxy-7-(4-(methylamino)-4,5,6,7-tetrahydro-1-benzothien-2-yl)-4-oxo-1,4-dihydroquinoline-3-carboxylic acid;

7-(4-amino-4,5,6,7-tetrahydro-1-benzothien-2-yl)-1-(2,4-difluorophenyl)-6-fluoro-8-methoxy-4-oxo-1,4-dihydroquinoline-3-carboxylic acid;

1-(2,4-difluorophenyl)-7-(4-(methylamino)-4,5,6,7-tetrahydro-1-benzothien-2-yl)-4-oxo-1,4-dihydro-1,8-naphthyridine-3-carboxylic acid;

7-(4-amino-4,5,6,7-tetrahydro-1-benzothien-2-yl)-1-cyclopropyl-6-fluoro-4-oxo-1,4-dihydroquinoline-3-carboxylic acid;

7-(4-amino-4,5,6,7-tetrahydro-1-benzothien-2-yl)-1-cyclopropyl-6,8-difluoro-4-oxo-1,4-dihydroquinoline-3-carboxylic acid;

7-(7-amino-4,5,6,7-tetrahydro-1-benzothien-2-yl)-1-cyclopropyl-6,8-difluoro-4-oxo-1,4-dihydroquinoline-3-carboxylic acid;

7-(7-amino-4,5,6,7-tetrahydro-1-benzothien-2-yl)-1-cyclopropyl-6-fluoro-4-oxo-1,4-dihydroquinoline-3-carboxylic acid;

1-cyclopropyl-7-(7-hydroxy-4,5,6,7-tetrahydro-1-benzothien-2-yl)-6-methyl-4-oxo-1,4-dihydroquinoline-3-carboxylic acid;

1-cyclopropyl-6-methyl-7-(4-(methylamino)-4,5,6,7-tetrahydro-1-benzothien-2-yl)-4-oxo-1,4-dihydroquinoline-3-carboxylic acid;

7-(7-amino-4,5,6,7-tetrahydro-1-benzothien-2-yl)-1-(2,4-difluorophenyl)-8-methoxy-4-oxo-1,4-dihydroquinoline-3-carboxylic acid;

8-chloro-1-cyclopropyl-7-(4-hydroxy-4,5,6,7-tetrahydro-1-benzothien-2-yl)-4-oxo-1,4-dihydroquinoline-3-carboxylic acid;

8-chloro-1-cyclopropyl-7-(7-hydroxy-4,5,6,7-tetrahydro-1-benzothien-2-yl)-4-oxo-1,4-dihydroquinoline-3-carboxylic acid;

1-cyclopropyl-6-fluoro-7-(4-hydroxy-4,5,6,7-tetrahydro-1-benzothien-2-yl)-8-methyl-4-oxo-1,4-dihydroquinoline-3-carboxylic acid;

1-cyclopropyl-6-fluoro-8-methyl-7-(4-(methylamino)-4,5,6,7-tetrahydro-1-benzothien-2-yl)-4-oxo-1,4-dihydroquinoline-3-carboxylic acid;

7-(4-amino-4,5,6,7-tetrahydro-1-benzothien-2-yl)-1-(2,4-difluorophenyl)-8-methoxy-4-oxo-1,4-dihydroquinoline-3-carboxylic acid;

5-amino-1-cyclopropyl-6,8-difluoro-7-(7-hydroxy-4,5,6,7-tetrahydro-1-benzothien-2-yl)-4-oxo-1,4-dihydroquinoline-3-carboxylic acid;

1-cyclopropyl-6-fluoro-7-(7-hydroxy-4,5,6,7-tetrahydro-1-benzothien-2-yl)-4-oxo-1,4-dihydro-1,8-naphthyridine-3-carboxylic acid;

7-(4-amino-4,5,6,7-tetrahydro-1-benzothien-2-yl)-1-cyclopropyl-6-fluoro-8-methyl-4-oxo-1,4-dihydroquinoline-3-carboxylic acid;

1-cyclopropyl-7-(7-hydroxy-4,5,6,7-tetrahydro-1-benzothien-2-yl)-6-methyl-4-oxo-1,4-dihydro- 1 ,8-naphthyridine-3-carboxylic acid;

7-(4-amino-4,5,6,7-tetrahydro-1-benzothien-2-yl)-1-cyclopropyl-5,8-difluoro-4-oxo-1,4-dihydroquinoline-3-carboxylic acid;

7-(7-amino-4,5,6,7-tetrahydro-1-benzothien-2-yl)-1-cyclopropyl-5,8-difluoro-4-oxo-1,4-dihydroquinoline-3-carboxylic acid;

7-(7-amino-4,5,6,7-tetrahydro-1-benzothien-2-yl)-1-cyclopropyl-6-methyl-4-oxo-1,4-dihydroquinoline-3-carboxylic acid;

7-(4-amino-4,5,6,7-tetrahydro-1-benzothien-2-yl)-8-chloro-1-cyclopropyl-4-oxo-1,4-dihydroquinoline-3-carboxylic acid;

7-(7-amino-4,5,6,7-tetrahydro-1-benzothien-2-yl)-8-chloro-1-cyclopropyl-4-oxo-1,4-dihydroquinoline-3-carboxylic acid;

5-amino-7-(4-amino-4,5,6,7-tetrahydro-1-benzothien-2-yl)-1-cyclopropyl-6,8-difluoro-4-oxo-1,4-dihydroquinoline-3-carboxylic acid;

7-(7-amino-4,5,6,7-tetrahydro-1-benzothien-2-yl)-1-cyclopropyl-6-methyl-4-oxo-1,4-dihydro-1,8-naphthyridine-3-carboxylic acid;

7-(7-(benzylamino)-4,5,6,7-tetrahydro-1-benzothien-2-yl)-1-cyclopropyl-8-methoxy-4-oxo-1,4-dihydroquinoline-3-carboxylic acid;

1-cyclopropyl-8-methoxy-4-oxo-7-(7-((pyridin-3-ylmethyl)amino)-4,5,6,7-tetrahydro-1-benzothien-2-yl)-1,4-dihydroquinoline-3-carboxylic acid;

7-(4-amino-4,5,6,7-tetrahydro-1-benzothien-2-yl)-1-cyclopropyl-6-methyl-4-oxo-1,4-dihydroquinoline-3-carboxylic acid;

1-cyclopropyl-7-(4-hydroxy-4,5,6,7-tetrahydro-1-benzothien-2-yl)-6-methyl-4-oxo-1,4-dihydroquinoline-3-carboxylic acid;

7-(4-amino-4,5,6,7-tetrahydro-1-benzothien-2-yl)-1-cyclopropyl-6-methyl-4-oxo-1,4-dihydro-1,8-naphthyridine-3-carboxylic acid;

7-(7-amino-6,6-dimethyl-4,5,6,7-tetrahydro-1-benzothien-2-yl)-1-cyclopropyl-6-fluoro-4-oxo-1,4-dihydroquinoline-3-carboxylic acid;

1-cyclopropyl-6-fluoro-7-(7-hydroxy-6,6-dimethyl-4,5,6,7-tetrahydro-1-benzothien-2-yl)-4-oxo-1,4-dihydroquinoline-3-carboxylic acid;

7-(7-amino-6,6-dimethyl-4,5,6,7-tetrahydro-1-benzothien-2-yl)-1-cyclopropyl-6-fluoro-4-oxo-1,4-dihydro-1,8-naphthyridine-3-carboxylic acid;

1-cyclopropyl-6-fluoro-7-(7-hydroxy-6,6-dimethyl-4,5,6,7-tetrahydro-1-benzothien-2-yl)-4-oxo-1,4-dihydro-1,8-naphthyridine-3-carboxylic acid;

5-amino-7-(7-amino-4,5,6,7-tetrahydro-1-benzothien-2-yl)-1-cyclopropyl-6,8-difluoro-4-oxo-1,4-dihydroquinoline-3-carboxylic acid;

5-amino-1-cyclopropyl-6,8-difluoro-7-(4-(methylamino)-4,5,6,7-tetrahydro-1-benzothien-2-yl)4-oxo-1,4-dihydroquinoline-3-carboxylic acid;

7-(7-amino-6,6-dimethyl-4,5,6,7-tetrahydro-1-benzothien-2-yl)-8-chloro-1-cyclopropyl-4-oxo-1,4-dihydroquinoline-3-carboxylic acid;

7-(7-amino-6,6-dimethyl-4,5,6,7-tetrahydro-1-benzothien-2-yl)-1-cyclopropyl-6-fluoro-8-methoxy-4-oxo-1,4-dihydroquinoline-3-carboxylic acid;

5-amino-7-(7-amino-4,5,6,7-tetrahydro-1-benzothien-2-yl)-1-cyclopropyl-8-fluoro-4-oxo-1,4-dihydroquinoline-3-carboxylic acid;

5-amino-1-cyclopropyl-8-fluoro-7-(7-hydroxy-4,5,6,7-tetrahydro-1-benzothien-2-yl)-4-oxo-1,4-dihydroquinoline-3-carboxylic acid;

5-amino-1-cyclopropyl-8-fluoro-7-(4-hydroxy-4,5,6,7-tetrahydro-1-benzothien-2-yl)-4-oxo-1,4-dihydroquinoline-3-carboxylic acid;

5-amino-1-cyclopropyl-7-(4-hydroxy-4,5,6,7-tetrahydro-1-benzothien-2-yl)-4-oxo-1,4-dihydroquinoline-3-carboxylic acid;

5-amino-7-(7-amino-4,5,6,7-tetrahydro-1-benzothien-2-yl)-6-chloro-1-cyclopropyl-8-fluoro-4-oxo-1,4-dihydroquinoline-3-carboxylic acid;

5-amino-6-chloro-1-cyclopropyl-8-fluoro-7-(7-hydroxy-4,5,6,7-tetrahydro-1-benzothien-2-yl)-4-oxo-1,4-dihydroquinoline-3-carboxylic acid;

5-amino-6-chloro-1-cyclopropyl-8-fluoro-7-(4-hydroxy-4,5,6,7-tetrahydro-1-benzothien-2-yl)-4-oxo-1,4-dihydroquinoline-3-carboxylic acid;

8-chloro-1-cyclopropyl-6-fluoro-7-(7-hydroxy-4,5 ,6,7-tetrahydro-1-benzothien-2-yl)-4-oxo-1,4-dihydroquinoline-3-carboxylic acid;

8-chloro-1-cyclopropyl-6-fluoro-7-(4-(methylamino)-4,5,6,7-tetrahydro-1-benzothien-2-yl)-4-oxo-1,4-dihydroquinoline-3-carboxylic acid;

8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid;

8-chloro-1-cyclopropyl-7-(4-(methylamino)-4,5,6,7-tetrahydro-1-benzothien-2-yl)-4-oxo-1,4-dihydroquinoline-3-carboxylic acid;

7-(7-amino-4,5,6,7-tetrahydro-1-benzothien-2-yl)-1-cyclopropyl-8-(difluoromethoxy)-6-fluoro-4-oxo-1,4-dihydroquinoline-3-carboxylic acid;

1-cyclopropyl-8-(difluoromethoxy)-6-fluoro-7-(7-hydroxy-6,6-dimethy-4,5,6,7-tetrahydro-1-benzothien-2-yl)-4-oxo-1,4-dihydroquinoline-3-carboxylic acid;

1-cyclopropyl-6-fluoro-7-(4-hydroxy-4,5,6,7-tetrahydro-1-benzothien-2-yl)-5-methyl-4-oxo-1,4-dihydro-1,8-naphthyridine-3-carboxylic acid;

7-(4-amino-4,5,6,7-tetrahydro-1-benzothien-2-yl)- 1 -cyclopropyl-6-fluoro-5-methyl-4-oxo-1,4-dihydro-1,8-naphthyridine-3-carboxylic acid;

7-(7-amino-4,5,6,7-tetrahydro-1-benzothien-2-yl)-1-cyclopropyl-6-fluoro-8-methyl-4-oxo-1,4-dihydroquinoline-3-carboxylic acid;

1-cyclopropyl-6-fluoro-7-(7-hydroxy-4,5,6,7-tetrahydro-1-benzothien-2-yl)-8-methyl-4-oxo-1,4-dihydroquinoline-3-carboxylic acid;

1-cyclopropyl-6-fluoro-7-(7-hydroxy-4,5,6,7-tetrahydro-1-benzothien-2-yl)-5-methyl-4-oxo-1,4-dihydro-1,8-naphthyridine-3-carboxylic acid;

7-(7-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid;

7-(7-amino-6,6-dimethyl-4,5,6,7-tetrahydro-1-benzothien-2-yl)-1-cyclopropyl-6-fluoro-8-methyl-4-oxo-1,4-dihydroquinoline-3-carboxylic acid; and

7-(7-amino-6,6-dimethyl-4,5,6,7-tetrahydro-1-benzothien-2-yl)-1-cyclopropyl-6,8-difluoro-4-oxo-1,4-dihydroquinoline-3-carboxylic acid.

The following additional compounds, representative of formula (I), may be prepared by one skilled in the art using known synthetic methodology or by using synthetic methodology described in the Schemes and Examples contained herein.

7-(7-amino-4,5,6,7-tetrahydro-1-benzothien-2-yl)-1-cyclopropyl-8-methyl-4-oxo-1,4-dihydroquinoline-3-carboxylic acid;

1-cyclopropyl-7-(7-hydroxy-4,5,6,7-tetrahydro-1-benzothien-2-yl)-8-methyl-4-oxo-1,4-dihydroquinoline-3-carboxylic acid;

7-(7-amino-6,6-dimethyl-4,5,6,7-tetrahydro-1-benzothien-2-yl)-1-cyclopropyl-8-methyl-4-oxo-1,4-dihydroquinoline-3-carboxylic acid;

1-cyclopropyl-7-(7-hydroxy-6,6-dimethyl-4,5,6,7-tetrahydro-1-benzothien-2-yl)-8-methyl-4-oxo-1,4-dihydroquinoline-3-carboxylic acid;

7-(4-amino-4,5,6,7-tetrahydro-1-benzothien-2-yl)-1-cyclopropyl-8-methyl-4-oxo-1,4-dihydroquinoline-3-carboxylic acid;

1-cyclopropyl-7-(4-hydroxy-4,5,6,7-tetrahydro-1-benzothien-2-yl)-8-methyl-4-oxo-1,4-dihydroquinoline-3-carboxylic acid;

1-cyclopropyl-8-methyl-7-[4-(methylamino)-4,5,6,7-tetrahydro-1-benzothien-2-yl]-4-oxo-1,4-dihydroquinoline-3-carboxylic acid;

7-(7-amino-4,5,6,7-tetrahydro-1-benzothien-2-yl)-1-cyclopropyl-6-fluoro-5,8-dimethyl-4-oxo-1,4-dihydroquinoline-3-carboxylic acid;

1-cyclopropyl-6-fluoro-7-(7-hydroxy-4,5,6,7-tetrahydro-1-benzothien-2-yl)-5,8-dimethyl-4-oxo-1,4-dihydroquinoline-3-carboxylic acid;

5-amino-7-(4-amino-4,5,6,7-tetrahydro-1-benzothien-2-yl)-6-chloro-1-cyclopropyl-8-fluoro-4-oxo-1,4-dihydroquinoline-3-carboxylic acid;

5-amino-7-(7-amino-6,6-dimethyl-4,5,6,7-tetrahydro-1-benzothien-2-yl)-6-chloro-1-cyclopropyl-8-fluoro-4-oxo-1,4-dihydroquinoline-3-carboxylic acid;

5-amino-7-(4-amino-4,5,6,7-tetrahydro-1-benzothien-2-yl)-8-chloro-1-cyclopropyl-6-fluoro-4-oxo-1,4-dihydroquinoline-3-carboxylic acid;

5-amino-7-(7-amino-4,5,6,7-tetrahydro-1-benzothien-2-yl)-8-chloro-1-cyclopropyl-6-fluoro-4-oxo-1,4-dihydroquinoline-3-carboxylic acid;

5-amino-7-(7-amino-6,6-dimethyl-4,5,6,7-tetrahydro-1-benzothien-2-yl)-8-chloro-1-cyclopropyl-6-fluoro-4-oxo-1,4-dihydroquinoline-3-carboxylic acid;

5-amino-8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid;

5-amino-8-chloro-1-cyclopropyl-6-fluoro-7-(7-hydroxy-4,5,6,7-tetrahydro-1-benzothien-2-yl)-4-oxo-1,4-dihydroquinoline-3-carboxylic acid;

7-(4-amino-4,5,6,7-tetrahydro-1-benzothien-2-yl)-8-chloro-1-cyclopropyl-6-fluoro-4-oxo-1,4-dihydroquinoline-3-carboxylic acid;

7-(7-amino-4,5,6,7-tetrahydro-1-benzothien-2-yl)-8-chloro-1-cyclopropyl-6-fluoro-4-oxo-1,4-dihydroquinoline-3-carboxylic acid;

7-(7-amino-6,6-dimethyl-4,5,6,7-tetrahydro-1-benzothien-2-yl)-8-chloro-1-cyclopropyl-6-fluoro-4-oxo-1,4-dihydroquinoline-3-carboxylic acid;

5-amino-7-(7-amino-6,6-dimethyl-4,5,6,7-tetrahydro-1-benzothien-2-yl)-1-cyclopropyl-6,8-difluoro-4-oxo-1,4-dihydroquinoline-3-carboxylic acid;

5-amino-1-cyclopropyl-6-fluoro-7-(4-hydroxy-4,5,6,7-tetrahydro-1-benzothien-2-yl)-8-methyl-4-oxo-1,4-dihydroquinoline-3-carboxylic acid;

5-amino-7-(4-amino-4,5,6,7-tetrahydro-1-benzothien-2-yl)-1-cyclopropyl-6-fluoro-8-methyl-4-oxo-1,4-dihydroquinoline-3-carboxylic acid;

5-amino-1-cyclopropyl-6-fluoro-8-methyl-7-[4-(methylamino)-4,5,6,7-tetrahydro-1-benzothien-2-yl]-4-oxo-1,4-dihydroquinoline-3-carboxylic acid;

5-amino-7-(7-amino-4,5,6,7-tetrahydro-1-benzothien-2-yl)-1-cyclopropyl-6-fluoro-8-methyl-4-oxo-1,4-dihydroquinoline-3-carboxylic acid;

5-amino-1-cyclopropyl-6-fluoro-7-(7-hydroxy-4,5,6,7-tetrahydro-1-benzothien-2-yl)-8-methyl-4-oxo-1,4-dihydroquinoline-3-carboxylic acid;

5-amino-7-(7-amino-6,6-dimethyl-4,5,6,7-tetrahydro-1-benzothien-2-yl)-1-cyclopropyl-6-fluoro-8-methyl-4-oxo-1,4-dihydroquinoline-3-carboxylic acid;

5-amino-1-cyclopropyl-7-(4-hydroxy-4,5,6,7-tetrahydro-1-benzothien-2-yl)-8-methyl-4-oxo-1,4-dihydroquinoline-3-carboxylic acid;

5-amino-7-(4-amino-4,5,6,7-tetrahydro-1-benzothien-2-yl)-1-cyclopropyl-8-methyl-4-oxo-1,4-dihydroquinoline-3-carboxylic acid;

5-amino-1-cyclopropyl-8-methyl-7-[4-(methylamino)-4,5,6,7-tetrahydro-1-benzothien-2-yl]-4-oxo-1,4-dihydroquinoline-3-carboxylic acid;

5-amino-1-cyclopropyl-7-(7-hydroxy-4,5,6,7-tetrahydro-1-benzothien-2-yl)-8-methyl-4-oxo-1,4-dihydroquinoline-3-carboxylic acid;

5-amino-7-(7-amino-4,5,6,7-tetrahydro-1-benzothien-2-yl)-1-cyclopropyl-8-methyl-4-oxo-1,4-dihydroquinoline-3-carboxylic acid; and

5-amino-7-(7-amino-6,6-dimethyl-4,5,6,7-tetrahydro-1-benzothien-2-yl)-1-cyclopropyl-8-methyl-4-oxo-1,4-dihydroquinoline-3-carboxylic acid.

Assay A. Determination of Biological Activity In Vitro Assay of Antibacterial Activity

Representative compounds of the invention were assayed in vitro for antibacterial activity. Twelve petri dishes containing successive aqueous dilutions of the test compound mixed with sterilized Brain Heart Infusion (BHI) agar (Difco 0418-01-5) (10 mL) were prepared. Each plate was inoculated with 1:100 (or 1:10 for slow-growing strains, such as Micrococcus and Streptococcus) dilutions of up to 32 different microorganisms using a Steers replicator block. The inoculated plates were incubated at 35-37° C. for about 20 to about 24 hours. In addition, a control plate with BHI agar and no test compound was prepared and incubated at the beginning and end of each test.

An additional plate containing a compound having known susceptibility patterns for the organisms being tested and belonging to the same antibiotic class as the test compound was also prepared and incubated as a further control and to provide test-to-test comparability. Ciprofloxacin was used for this purpose.

After incubation, each plate was visually inspected. The minimum inhibitory concentration (MIC) was defined as the lowest concentration of drug yielding no growth, a slight haze, or sparsely isolated colonies on the inoculum spot as compared to the growth control. The results of this assay, shown in Table 1, demonstrate the antibacterial activity of the compounds of the invention.

| Microorganism | Code |
|---|---|
| Staphylococcus aureus ATCC 6538P | A |
| Enterococcus faecalis PIU 1967 | B |
| Moraxella catarrahalis 2604 | C |
| Escherichia coli JUHL | D |
| Haemophilus influenzae DILL AMP R | E |
| Streptococcus pneumoniae ATCC 6303 | F |

TABLE 1
Antibacterial Activity (MIC's) of Selected Compounds

| | A | B | C | D | E | F |
|---|---|---|---|---|---|---|
| Example 2 | 0.05 | 0.2 | 0.2 | 0.2 | 0.12 | 0.5 |
| Example 7 | 0.2 | 0.39 | 0.2 | 0.2 | 0.06 | 0.25 |
| Example 9 | 0.1 | 0.2 | 0.1 | 0.1 | 0.06 | 0.125 |
| Example 13 | 0.1 | 0.2 | 0.2 | 0.2 | 0.25 | 0.25 |
| Example 20 | 0.2 | 0.78 | 0.78 | 0.78 | 0.25 | 1 |
| Example 22 | 0.2 | 0.78 | 0.39 | 0.78 | 0.25 | 2 |
| Example 28 | 0.2 | 0.78 | 0.2 | 0.78 | 0.25 | 1 |
| Example 32 | 0.2 | 0.39 | 0.2 | 0.39 | 0.25 | 1 |
| Example 35 | 0.05 | 0.78 | 0.05 | 0.78 | 0.06 | 0.5 |
| Example 37 | 0.05 | 0.1 | 0.1 | 0.2 | 0.125 | 0.25 |
| Example 40 | 0.02 | 0.1 | 0.1 | 0.2 | 0.06 | 0.125 |
| Example 42 | 0.02 | 0.39 | 0.02 | 0.78 | 0.125 | 0.25 |
| Example 45 | 0.05 | 0.2 | 0.1 | 0.39 | 0.03 | 0.25 |
| Example 47 | 0.01 | 0.2 | 0.02 | 1.56 | 0.5 | 0.5 |
| Example 48 | 0.02 | 0.2 | 0.02 | 0.78 | 0.06 | 0.25 |
| Example 49 | 0.05 | 0.39 | 0.05 | 0.78 | 0.06 | 0.25 |
| Example 52 | 0.2 | 0.78 | 0.2 | 0.39 | 0.25 | 0.25 |

Assay B. Determination of Biological Activity In Vitro Assay of Antibacterial Activity

Representative compounds of the invention were assayed in vitro for antibacterial activity. The susceptibilities of aerobic, nonfastidious species were determined visually by broth microdilution as described by the National Committee for Clinical Laboratory Standards (NCCLS) (National Committee for Clinical Laboratory Standards. 1997. Methods for dilution antimicrobial susceptibility tests for bacteria that grow aerobically. Approved standard M7-A4. National Committee for Clinical Laboratory Standards. Wayne, Pa.). In brief, serial two-fold dilutions of test compounds were made in cation-adjusted Mueller Hinton broth at two-times the final concentration. Inocula were prepared from overnight cultures of test strains. Tests were inoculated with a suspension of bacteria in Mueller Hinton broth to achieve a final density of approximately 5×10⁵ CFU/mL. Tests were incubated at 35° C. in ambient air for 16 to 20 hr. Minimum Inhibitory Concentrations (MICs) were determined as the lowest drug concentration completely inhibiting growth. Ciprofloxacin was included as a control. For testing S. pneumoniae, the cation-adjusted Mueller Hinton broth was supplemented with 3% lysed horse blood and the incubation period was extended to 20 to 24 hr. For testing H. influenzae, the cation-adjusted Mueller Hinton broth was supplemented with 5 g/L yeast extract, 0.3 mmol/L hematin and 15 mg/L NAD and incubation was extended to 20 to 24 hr.. The results of this assay, shown in Table 2, demonstrate the antibacterial activity of the compounds of the invention.

| Microorganism | Code |
|---|---|
| Staphylococcus aureus ATCC 6538P | A |
| Enterococcus faecalis PIU 1967 | B |
| Escherichia coli JUHL | D |
| Haemophilus influenzae DILL AMP R | E |
| Streptococcus pneumoniae ATCC 6303 | F |

TABLE 2
Antibacterial Activity (MIC's) of Selected Compounds

| | | | | |
|---|---|---|---|---|
| A | B | D | E | F |

| | | | | | |
|---|---|---|---|---|---|
| Example 59 | 0.03 | 1 | 8 | 1 | 0.5 |
| Example 66 | 0.06 | 0.5 | 2 | 0.5 | 0.5 |
| Example 110 | 0.004 | 0.06 | 0.25 | 0.015 | 0.06 |
| Example 126 | 0.004 | 0.5 | 16 | 1 | 0.5 |
| Example 127 | 0.5 | 0.25 | 1 | 0.125 | 0.5 |
| Example 130 | NA | 0.125 | 0.06 | 0.015 | 0.125 |
| Example 200 | 0.03 | 0.125 | 0.06 | 0.03 | 0.125 |
| Example 202 | 0.125 | 0.5 | 0.125 | 0.015 | 0.25 |
| Example 208 | 0.125 | 0.5 | 0.25 | 0.06 | 0.25 |
| Example 209 | 0.125 | 0.5 | 0.25 | 0.03 | 0.25 |
| Example 225 | 0.5 | 0.5 | 1 | 0.125 | 0.25 |
| Example 230 | 0.125 | 1 | 1 | 0.25 | 0.125 |
| Example 53 | 0.06 | 2 | 8 | 1 | 0.5 |
| Example 76 | 0.015 | 0.06 | 0.06 | 0.03 | 0.03 |
| Example 125 | 0.004 | 0.25 | 0.125 | 0.03 | 0.03 |
| Example 201 | 0.015 | 0.06 | 0.125 | 0.03 | 0.03 |
| Example 216 | 0.06 | 0.25 | 0.25 | 0.125 | 0.125 |
| Example 228 | 0.008 | 0.03 | 0.06 | 0.015 | 0.015 |
| Example 232 | 0.06 | 0.5 | 0.25 | 0.125 | 0.125 |
| Example 237 | 0.015 | 0.5 | 0.125 | 0.03 | 0.125 |
| Example 240 | 0.004 | 0.06 | 0.5 | 0.03 | 1 |
| Example 243 | 0.015 | 0.25 | 1 | 0.25 | 1 |
| Example 246 | 0.004 | 0.25 | 0.25 | 0.03 | 0.06 |
| Example 64 | 0.03 | 0.25 | 2 | 0.25 | 0.125 |
| Example 68 | 0.008 | 0.125 | 0.5 | 0.06 | 0.125 |
| Example 73 | 0.015 | 0.06 | 0.5 | 0.03 | 0.06 |
| Example 74 | 0.015 | 0.25 | 0.5 | 0.06 | 0.125 |
| Example 80 | 32 | >64 | >64 | 0.06 | 0.03 |
| Example 84 | 0.06 | 0.25 | 1 | 0.125 | 0.25 |
| Example 85 | 0.03 | 0.25 | 0.5 | 0.125 | 0.125 |
| Example 96 | <0.004 | 0.5 | 1 | 0.03 | 0.03 |
| Example 100 | 0.06 | 0.5 | 0.25 | 0.125 | 0.03 |
| Example 105 | 0.03 | 0.25 | 1 | 0.06 | 0.03 |
| Example 131 | — | 0.5 | 1 | 0.125 | 0.5 |
| Example 182 | 0.03 | 0.25 | 1 | 0.125 | 0.25 |
| Example 197 | 0.06 | 0.5 | 2 | 0.5 | 0.5 |
| Example 213 | 0.03 | 0.5 | 0.5 | 0.25 | 0.5 |
| Example 233 | 0.03 | 0.5 | 8 | 0.5 | 2 |
| Example 61 | 0.03 | 0.25 | 2 | 0.125 | 0.125 |
| Example 63 | 0.015 | 0.125 | 0.05 | 0.06 | 0.25 |
| Example 92 | 0.03 | 0.25 | 1 | 0.06 | 0.5 |
| Example 106 | 0.08 | 0.125 | 0.5 | 0.06 | 0.125 |
| Example 185 | 0.015 | 0.25 | 0.5 | 0.125 | 0.5 |
| Example 199 | 0.03 | 0.125 | 0.25 | 0.125 | 0.06 |
| Example 247 | 0.125 | 2 | 4 | 0.25 | 0.25 |
| Example 248 | 0.03 | 0.25 | 2 | 0.5 | 0.5 |
| Example 249 | 0.03 | 0.125 | 0.06 | 0.015 | 0.5 |
| Example 251 | 0.03 | 0.5 | 2 | 0.125 | 0.25 |
| Example 252 | 0.5 | 1 | 4 | 0.25 | 1 |
| Example 254 | 0.06 | 0.5 | 1 | 0.06 | 0.25 |
| Example 255 | 0.5 | 4 | 32 | 2 | 8 |
| Example 239 | 1 | 32 | 32 | 4 | >64 |
| Example 200 | 0.03 | 0.125 | 0.06 | 0.03 | 0.125 |
| Example 218 | 0.06 | 0.25 | 2 | 0.25 | 0.5 |
| Example 222 | 0.125 | 0.5 | 1 | 0.5 | 0.25 |
| Example 235 | 0.015 | 0.25 | 0.25 | 0.06 | 0.06 |
| Example 241 | 0.015 | 0.25 | 1 | 0.25 | 1 |
| Example 245 | 0.004 | 0.25 | 0.25 | 0.06 | 0.125 |
| Example 58 | 0.004 | 0.06 | 0.25 | 0.015 | 0.06 |
| Example 77 | 0.015 | 0.125 | 0.25 | 0.06 | 0.03 |
| Example 83 | 0.125 | 0.5 | 0.5 | 0.25 | 0.125 |
| Example 89 | 0.125 | 1 | 2 | 0.5 | 1 |
| Example 94 | 0.125 | 1 | 1 | 0.25 | 0.5 |
| Example 97 | 0.125 | 0.5 | 1 | 0.25 | 0.5 |
| Example 104 | 0.25 | 1 | 2 | 0.25 | 0.5 |
| Example 133 | 0.5 | 1 | 1 | 1 | 1 |
| Example 155 | 0.03 | 0.125 | 0.5 | 0.06 | 0.03 |
| Example 198 | 0.25 | 0.25 | 0.5 | 0.25 | 0.125 |
| Example 207 | 0.015 | 0.125 | 1 | 0.125 | 0.5 |
| Example 212 | 0.125 | 0.5 | 0.5 | 0.25 | 0.125 |
| Example 234 | 0.03 | 0.5 | 2 | 0.5 | 0.5 |
| Example 236 | 0.125 | 1 | 4 | 0.5 | 4 |
| Example 238 | 0.03 | 0.5 | 0.25 | 0.125 | 0.06 |
| Example 55 | <=0.004 | 0.125 | 0.5 | 0.06 | 0.125 |
| Example 56 | 0.03 | 0.5 | 4 | 0.25 | 0.125 |
| Example 70 | 0.03 | 0.03 | 0.125 | 0.015 | 0.06 |
| Example 76 | 0.015 | 0.06 | 0.06 | 0.03 | 0.03 |
| Example 78 | 0.125 | 1 | 4 | 2 | 0.25 |
| Example 79 | 0.125 | 1 | 4 | 1 | 0.5 |
| Example 244 | <=0.004 | 0.125 | 1 | 0.06 | 1 |
| Example 210 | 0.06 | 0.125 | 0.25 | 0.03 | 0.06 |
| Example 224 | 0.015 | 0.03 | 0.125 | 0.015 | 0.03 |
| Example 71 | 0.03 | 0.03 | 0.06 | 0.015 | 0.015 |
| Example 88 | 0.015 | 0.25 | 1 | 0.125 | 0.125 |
| Example 99 | 0.015 | 0.125 | 0.25 | 0.015 | 0.06 |
| Example 101 | 0.03 | 0.125 | 0.125 | 0.015 | 0.03 |
| Example 156 | 0.03 | 0.125 | 0.03 | 0.015 | 0.06 |
| Example 204 | <=0.004 | 0.03 | 0.125 | <=0.004 | 0.125 |
| Example 206 | 0.03 | 0.125 | 1 | 0.5 | 0.5 |
| Example 231 | 0.015 | 0.125 | 0.5 | 0.125 | 0.06 |
| Example 102 | 0.25 | 1 | 8 | 0.03 | 0.5 |
| Example 109 | 0.125 | 2 | 8 | 0.125 | 0.5 |
| Example 135 | 1 | 1 | 16 | 1 | 4 |
| Example 143 | 0.125 | 0.5 | 64 | 0.125 | 0.5 |
| Example 227 | 0.5 | 2 | >64 | 2 | 8 |
| Example 107 | 0.06 | 0.5 | 0.25 | 0.06 | 0.06 |
| Example 108 | 0.015 | 1 | 0.03 | 0.008 | 0.25 |
| Example 268 | 0.03 | 0.25 | 0.5 | 0.25 | 0.06 |
| Example 299 | <=0.004 | 0.06 | 0.125 | 0.03 | 0.06 |
| Example 342 | 0.03 | 0.25 | 0.5 | 0.25 | 0.125 |
| Example 296 | <=0.004 | 0.25 | 0.5 | 0.06 | 0.5 |
| Example 307 | 0.008 | 0.03 | 0.03 | 0.015 | 0.008 |
| Example 293 | <=0.004 | 0.008 | 0.015 | <=0.004 | 0.03 |
| Example 310 | 0.008 | 0.015 | 0.015 | 0.015 | 0.008 |
| Example 290 | 0.03 | 0.125 | 0.03 | 0.015 | 0.03 |
| Example 294 | <=0.004 | 0.015 | 0.03 | <=0.004 | 0.03 |
| Example 308 | 0.008 | 0.03 | 0.03 | 0.015 | 0.015 |
| Example 295 | <=0.004 | 0.008 | 0.03 | <=0.004 | 0.03 |
| Example 309 | 0.015 | 0.06 | 0.03 | 0.015 | 0.06 |
| Example 291 | 0.015 | 0.125 | 0.03 | 0.015 | 0.03 |
| Example 319 | <=0.004 | 0.06 | 0.06 | 0.015 | 0.03 |
| Example 346 | <=0.004 | 0.03 | 0.125 | 0.015 | 0.008 |
| Example 347 | 0.03 | 0.25 | 0.25 | 0.06 | 0.125 |
| Example 348 | 0.015 | 0.125 | 0.25 | 0.06 | 0.03 |
| Example 326 | 0.125 | 0.25 | 0.125 | 0.015 | 0.125 |
| Example 349 | 0.015 | 0.125 | 0.03 | <=0.004 | 0.03 |
| Example 271 | <=0.004 | 0.015 | <=0.004 | <=0.004 | <=0.004 |
| Example 282 | 0.008 | 0.03 | 0.015 | 0.03 | 0.015 |
| Example 267 | <=0.004 | 0.125 | 0.5 | 0.06 | 0.25 |
| Example 287 | 0.03 | 0.125 | 0.06 | 0.03 | 0.03 |
| Example 281 | 0.03 | 0.125 | 0.125 | 0.06 | 0.25 |
| Example 288 | 0.03 | 0.125 | 0.03 | 0.015 | 0.03 |
| Example 280 | 0.015 | 0.125 | 0.125 | 0.06 | 0.25 |
| Example 329 | <=0.004 | 0.03 | 0.03 | 0.008 | 0.008 |
| Example 361 | 0.015 | 0.06 | 0.06 | 0.015 | 0.03 |
| Example 330 | 0.015 | 0.06 | 0.03 | 0.008 | 0.03 |
| Example 315 | 0.008 | 0.06 | 0.125 | 0.008 | 0.125 |
| Example 367 | 0.015 | 0.125 | 0.06 | 0.03 | 0.03 |
| Example 317 | 0.03 | 0.06 | 0.06 | 0.03 | 0.03 |
| Example 321 | <=0.004 | 0.015 | 0.015 | <=0.004 | <=0.004 |
| Example 316 | <=0.004 | 0.06 | 0.06 | <=0.004 | 0.03 |
| Example 368 | 0.015 | 0.06 | 0.03 | <=0.004 | 0.03 |
| Example 369 | <=0.004 | 0.03 | 0.06 | <=0.004 | 0.03 |
| Example 371 | <=0.004 | 0.03 | 0.06 | 0.03 | <=0.004 |

Abbreviations

Abbreviations which have been used in the descriptions of the schemes and the examples that follow are: m-CPBA for meta-chloroperbenzoic acid; dba for dibenzylidine acetone; dba for dibenzylidine acetone; Ac for acetyl; CDI for carbonyldiimidazole; DMAP for 4-(N,N-dimethylamino)pyridine; DME for dimethoxyethane; DMF for N,N-dimethylformamide; DMS for dimethylsulfide; DMSO for dimethylsulfoxide; EDCI for 1-((3-(dimethylamino)propyl)-3-ethylcarbodiimide hydrochloride; NMP for 1-methyl-2-pyrrolidinone; TEA for triethylamine; TBS for tert-butyldimethylsilyl; TFA for trifluoroacetic acid; and THF for tetrahydrofuran.

Synthetic Methods

The compounds and processes of the invention will be better understood in connection with the following synthetic schemes which illustrate methods by which the compounds can be prepared. The compounds can be prepared by a variety of synthetic routes. Representative procedures are shown in Schemes 1-6. It will be readily apparent that the compounds can be synthesized by substitution of the appropriate reactants and agents in the syntheses shown below. It will also be apparent that the selective protection and deprotection steps, as well as the order of the steps themselves, can be carried out in varying order, depending on the nature of the reactions. A thorough discussion of protecting groups is provided in Greene and Wuts, “Protective Groups in Organic Synthesis,” 2nd Ed., John Wiley & Son, Inc., 1991.

Precursor compounds, intermediates, and reagents are commercially available or can be prepared from commercially available starting materials. Functional group transformations useful for preparing compounds of the invention are reported in Larock, “Comprehensive Organic Transformations. A Guide to Functional Group Preparations,” VCH Publishers, New York (1989). The groups A¹, A², Q¹, Q², R¹, R², R³, R⁴, R⁵, R⁶, R⁷, R⁸, R⁹, R¹⁰, R¹², R¹³, R¹⁴, R¹⁵, R¹⁶, W, and Z are as defined above unless otherwise noted below. In the following schemes, the group R^{11a} is a carboxyl protecting group.

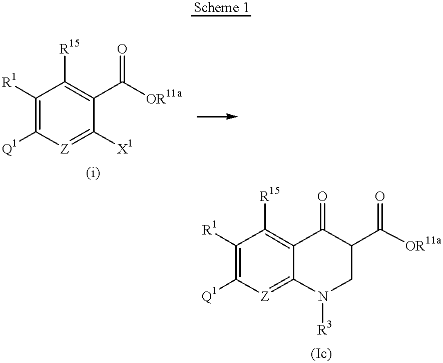

A synthesis of compounds of formula (Ic) is shown in Scheme 1. Conversion of compounds of formula (i), wherein X¹ is halide, to compounds of formula (Ic) can be achieved by means such as those reported in U.S. Pat. No. 5,282,703. The starting materials are available commercially or can be prepared by well-known means. For example, commercially available 4-bromo-2-fluorobenzoic acid (Alfa/Aesar, Ward Hill, Mass.), ethyl 3-hydroxybenzoate, and 2,6-dichloronicotinic acid (Aldrich Chemical Company, Milwaukee, Wis.) ultimately provide compounds of formula (Ic). A list of additional starting materials (i) with references for their preparation is as follows:

3-amino-5-hydroxybenzoic acid Tetrahedron 39(24), 4189 (1989);

2,4-dibromo-3-methoxybenzoic acid WO 9616046;

4-bromo-2,5-difluoro-3-methoxybenzoic acid WO 9605192;

methyl 2,6-dichloro-5-methyl-nicotinate Tetrahed. Lett. 1989, 30, 3183-3186;

2,6-difluoro-4-bromobenzoic acid Tetrahed. Lett. 1996, 37, 6551-6554;

4-bromo-2,3,6-trifluorobenzoic acid, prepared via metalation and carboxylation of 1-bromo-2,3,5-trifluorobenzene (Lancaster Synthesis); and

4-bromo-2-fluoro-5-methylbenzoic acid, prepared via sodium hypochlorite oxidation of 1-(4-bromo-2-fluoro-5-methyl-phenyl)ethanone, prepared by the method disclosed in WO9602486.

An alternative method of preparing compounds of formula (Ic) is disclosed in U.S. Pat. No. 6,025,370, which can be employed to prepare derivatives, such as, for example, ethyl 7-bromo-1-cyclopropyl-8-methyl-4-oxo-1,4-dihydroquinoline-3-carboxylate.

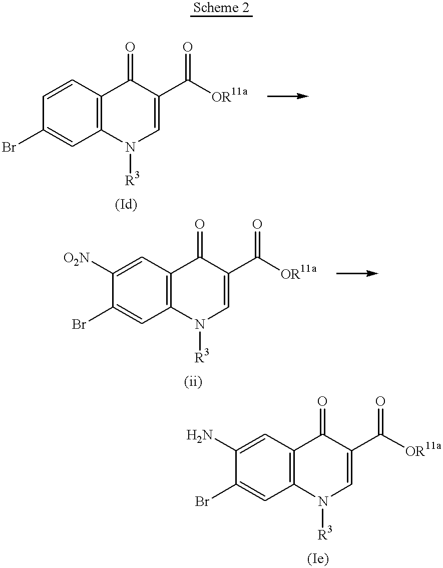

As shown in Scheme 2, compounds of formula (Id) can be nitrated with fuming nitric acid in sulfuric acid to provide compounds of formula (ii). Conversion of compounds of formula (ii) to compounds of formula (Ie) can be achieved with metal powder such as zinc, iron, or tin in acids such as hydrochloric, hydrobromic, sulfuric, acetic, trifluoroacetic, or mixtures thereof. Additional compounds of formula (Ie) can be prepared by alkylating the amino group at the C-6 position of the compounds of formula (Ie) with alkyl chlorides, bromides, or iodides in the presence of a base such as cesium carbonate, potassium bicarbonate, sodium hydride, or potassium hydride.

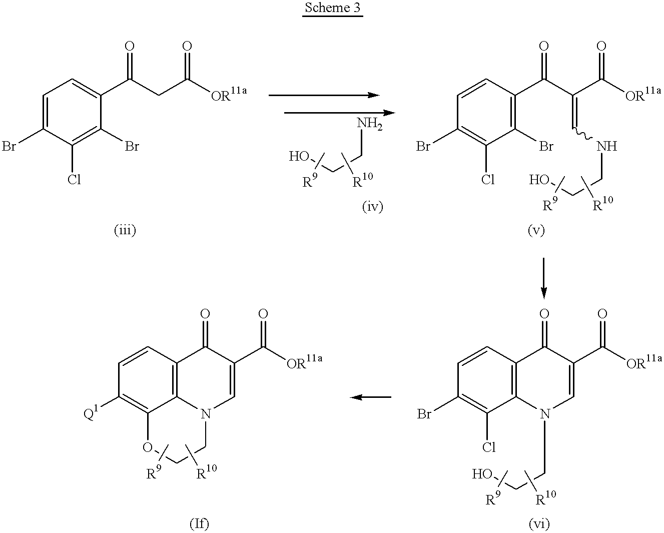

A synthesis of compounds of formula (If) wherein R² and R³ are taken together is shown in Scheme 3. Conversion of ehtyl 3-aminobenzoate to ethyl 3-amino-2,4-dibromobenzoate can be achieved by means well-known in the art. The ethyl 3-amino-2,4-dibromobenzoate can then be converted to ethyl 2,4-dibromo-3-chlorobenzoate by diazotization/chlorination according to the procecure described in Chem. Pharm. Bull. 37(8) 2103 (1989) or alternatively converted ethyl 3-amino-2,4-dibromobenzoate by diazotization/reduction as described in J. Am. Chem. Soc. 72, 3013 (1950) and J. Org. Chem. 42, 1469 (1977). Each of these substituted benzoic acids can then be elaborated to compounds of formulas (v), (vi), and (Ic) as described in Scheme 1 and in U.S. Pat. Nos. 4,382,892 and 4,762,831.

The functionalized amines of formula (iv) can be optionally protected amino acids, amino sulfhydryls, or amino alcohols, the latter two of which can be derived from amino acids of known stereochemistry by preparations such as those described in Tetrahedron Lett. 36(8) 1223 (1995).

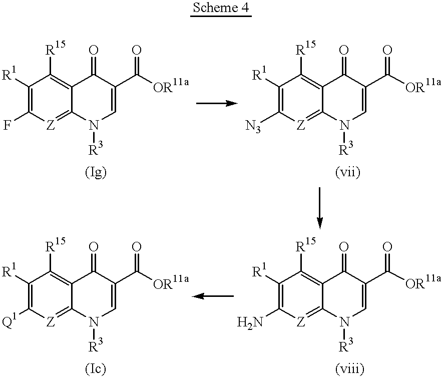

As shown in Scheme 4, compounds of formula (Ic) in which Q¹ is a first covalent bond precursor, for example, bromine, can be prepared from compounds of formula (Ig), which possesses a suitable leaving group at the quinolone C-7 position.

Compounds of formula (Ig) can be converted to compounds of formula (vii) by treatment of the former with an azido compound, for example sodium azide. Solvents useful for the reaction include polar aprotic solvents such as DMF, NMP, THF, and the like. The temperatures at which the reactions are conducted typically range from about 25° C. to about 100° C.

Conversion of compounds of formula (vii) to compounds of formula (viii) can be achieved by treatment of the former with hydrogen gas and a hydrogenation catalyst. Representative catalysts include palladium on carbon and palladium on alumina. Solvents useful for the reaction include methanol, ethanol, THF, 1,4-dioxane, and the like. The reaction is generally carried out at ambient temperature.

Conversion of compounds of formula (viii) to compounds of formula (Ic) can be achieved by diazotization in the presence of a copper salt. Representative copper salts include cuprous or cupric salts, including copper(II) bromide and copper(I) chloride. Solvents useful for the reaction include aqueous acids, for example dilute aqueous hydrobromic acid. The reaction is generally carried out at about 0° C.

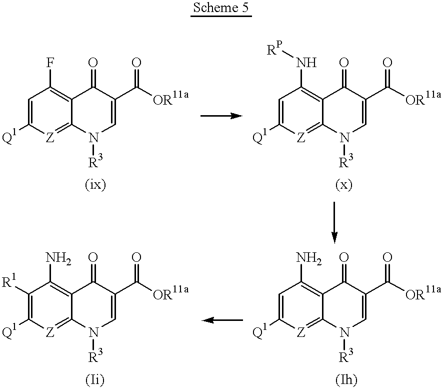

Scheme 5 is an alternative method for preparation of compounds with C-5 and C-6 substituents. Compounds of formula (ix) can undergo nucleophilic aromatic substitution at position C-5 by treatment with a protected amine to give compounds of formula (x), wherein R^{P} is an amino protecting group. Examples of protected amines include 2,4-dimethoxybenzylamine, benzylamine, and the like. Solvents useful for the reaction include trichloroethylene, which can be employed in a temperature range of from about 80° C. to about 85° C.

Deprotection of compounds of formula (x) to give compounds of formula (Ih) can be achieved by several standard means well known in the art. For example, treatment of a benzyl or substituted benzyl derivative under hydrogenation conditions or TFA give the desired aniline product.

Conversion of compounds of formula (Ih) to compounds of formula (Ii) can be achieved by treatment of the former with electrophilic reagents via electrophilic aromatic substitution. Representative electrophilic agents include 1,3-dichloro-5,5-dimethylhydantoin, sulfuryl chloride, N-bromosuccinimide, and the like. Solvents useful for the reaction include halogenated solvents including dichloromethane, chloroform, and the like.

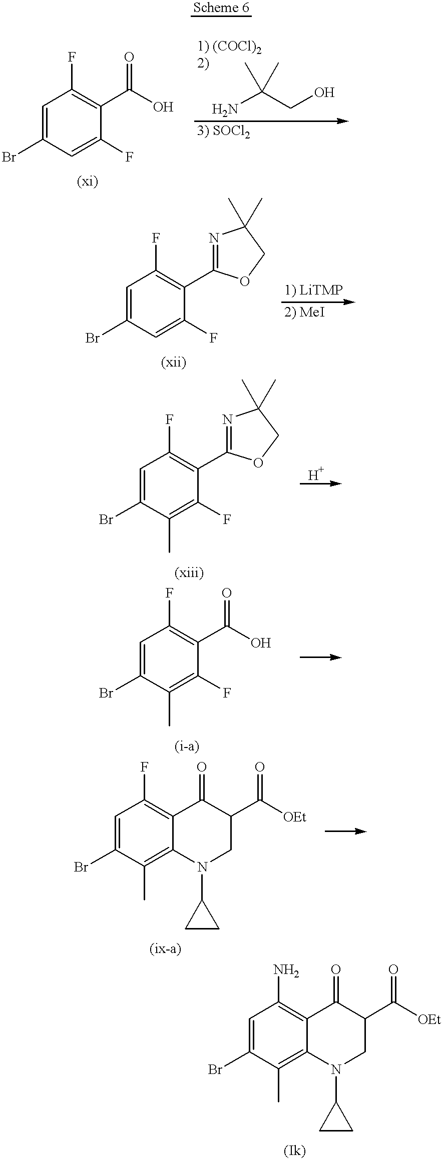

4-Bromo-2,6-difluorobenzoic acid (xi) (prepared by the method of Mongin, F; Schlosser, M. Tetrahedron Lett. 1996, 37, 6551) can be converted to its oxazole derivative (xii) under standard conditions, lithiated with an appropriate base such as lithium tetramethylpiperidide or the like, and alkylated with a suitable alkylating agent such as iodomethane, dimethyl sulfate, and the like to give (xiii). The resulting product can then be hydrolyzed under acidic conditions to provide the compound of formula (i-a). This acid can easily be converted to compound (ix-a) according to Scheme 1 and further converted to compound (Ik) according to Scheme 5.

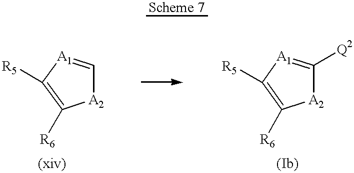

As shown in Scheme 7, compounds of formula (xiv), which are commercially available or prepared by means well-known in the art, can be converted to compounds of formula (Ib) by treatment with a base such as the lithium bis(trimethylsilyl)amide, sodium bis(trimethylsilyl)amide, potassium bis(trimethylsilyl)amide, n-butyllithium, sec-butyllithium, tert-butyllithium, and lithium diisopropylamide followed by treatment of the resulting anion with a Q² precursor such as bromine, N-bromosuccinimide, a trialkoxyborane, and a trialkylstannyl halide. For compounds of formula (Ib) wherein Q² is halide, the halide can be further derivatized by treatment with a metal such as magnesium or zinc to provide the corresponding magnesium halide or zinc halide. For compounds of formula (Ib), wherein Q² is a boronic ester, the boronic ester can be hydrolyzed to the boronic acid with a base such as lithium hydroxide, sodium hydroxide, and potassium hydroxide or an acid such as hydrochloric or hydrobromic.

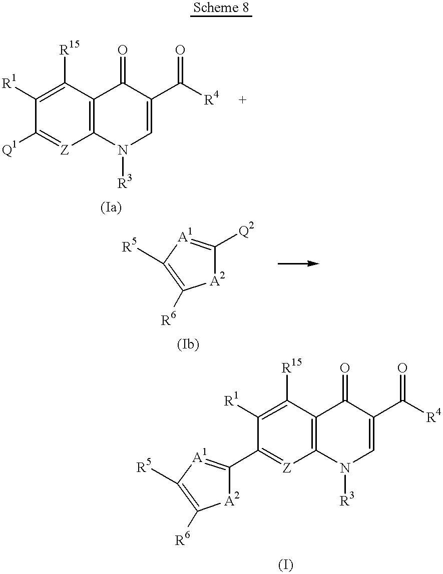

As shown in Scheme 8, compounds of formula (I) can be prepared from precursor compounds of formulas (Ia) and (Ib) in the presence of a catalyst. Examples of catalysts include tetrakis(triphenylphosphine)palladium(O), palladium(II) chloride(dibenzylidine acetone), and palladium(II) chloride bis(triphenylphosphine). If necessary, these reactions can be run on the presence of base such as Na₂CO₃, Cs₂CO₃, CsF, and K₂HPO₄, additives such as LiCl, or ligands such as triphenylphosphine, triphenylarsine, and trialkylphosphines such as tributylphosphine.

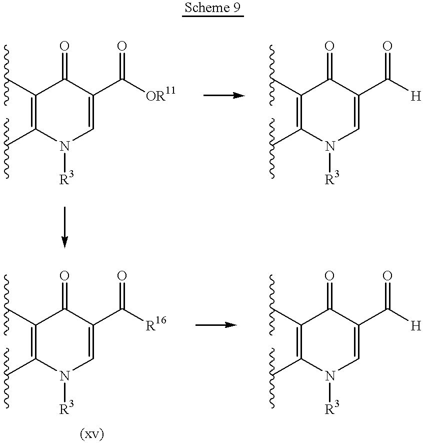

As shown in Scheme 9, the ester group of compounds of formula (I) can be directly converted to aldehydes by treatment of the former with a hydride donating agents such as diisobutylaluminum hydride. An alternative conversion of ester groups of compounds of formula (I) to aldehydes is conversion of the corresponding acid to a Weinreb amide, exemplified by compounds of formula (xv) wherein R¹⁶ is —N(CH₃)(OCH₃), followed by treatment with the aformentioned hydride donating agents.

Yet another alternative conversion of the ester group of compounds of formula (I) to aldehydes is conversion of the corresponding acid to a thioester followed by treatment of the thioester with a hydrogen source and a catalyst. Examples of hydrogen sources are hydrogen gas or triethylsilane. Examples of catalysts are palladium on carbon or platinum on carbon.

The compounds and processes of the present invention will be better understood in connection with the following examples, which are intended as an illustration of and not a limitation upon the scope of the invention.

### EXAMPLE 1

### 1-cyclopropyl-4-oxo-7-(4,5,6,7-tetrahydrothieno[3,2-c]pyridin-2-yl)-1,4-dihydro-3-quinolinecarboxylic acid hydrochloride

### EXAMPLE 1A

### N-methylene-2-(2-thienyl)ethanamine

A solution of 2-thiophenethylamine (10.0 g, 78.0 mmol) in 37% aqueous formaldehyde (2.81 g, 94.0 mmol) was heated to 100° C. for 3 hours, cooled to room temperature, and diluted with ethyl acetate. The layers separated, and the organic layer was washed with brine, dried (Na₂SO₄), filtered, and concentrated to provide 9.86 g (91%) of the desired product as a yellow oil. MS (DCI/NH₃) m/z 157 (M+H)⁺; ¹H NMR (300 MHz, CDCl₃) 7.11 (dd, 1H), 6.91 (m, 1H), 6.82 (dd, 1H), 3.47 (br s, 2H), 2.96 (t, 2H), 2.78 (t, 2H).

### EXAMPLE 1B

### 4,5,6,7-tetrahydrothieno[3,2-c]pyridine

A solution of Example 1A (17.7 g, 127 mmol) in 5.5M HCl (23 mL) was treated with concentrated HCl (3.1 mL), stirred for 3 hours, and concentrated. The residue was partitioned between 1M NaOH and ethyl acetate. The organic phase was washed with brine, dried (Na₂SO₄), filtered, and concentrated. The residue was purified by flash flash column chromotography on silica gel with 20% methanol in chloroform to provide 4.50 g (25%) of the desired product as a colorless oil. MS (DCI/NH₃) m/z 157 (M+18)⁺; ¹H NMR (300 MHz, CDCl₃) 7.05 (d, 1H), 6.74 (d, 1H), 3.93 (m, 2H), 3.15 (t, 2H), 2.81 (t, 2H).

### EXAMPLE 1C

### 5-trityl-4,5,6,7-tetrahydrothieno[3,2-c]pyridine

A solution of Example 1B (1.3 g, 9.3 mmol) in THF (5 mL) was added dropwise to a suspension of 60% oily NaH (0.632 g, 15.8 mmol) in THF (30 mL) at 0° C., stirred for 30 minutes, treated dropwise with a solution of triphenylmethyl chloride (2.95 g, 10.6 mmol) in THF (10 mL), stirred an additional 1 hour, and quenched with water. The resulting mixture was partitioned between ethyl acetate and water. The aqueous layer was extracted with ethyl acetate, and the combined extracts were washed with brine, dried (Na₂SO₄), filtered, and concentrated. The concentrate was purified by flash flash column chromatography on silica gel with 5% then 10% ethyl acetate in hexanes to provide 1.80 g (50%) of the desired product as a tan solid. MS (DCI/NH₃) m/z 382 (M+H)⁺; ¹H NMR (300 MHz, CDCl₃) 7.55 (d, 6H), 7.26 (m, 6H), 7.17 (m, 3H), 7.11 (d, 1H), 6.63 (d, 1H), 3.41 (br s, 2H), 2.98 (br s, 2H), 2.60 (br s, 2H).

### EXAMPLE 1D

### 2-(tributylstannyl)-5-trityl-4,5,6,7-tetrahydrothieno[3,2-c]pyridine

A solution of Example 1C (1.77 g, 4.60 mmol) in THF (50 mL) at −78° C. was treated dropwise with 2.5M n-butyllithium in hexanes (2.5 mL, 6.40 mmol), warmed to −10° C., stirred for 3 hours, cooled to −78° C., treated dropwise with a solution of chlorotributylstannane (1.65 g, 5.1 mmol) in THF (5 mL), warmed to room temperature, and partitioned between ethyl acetate and water. The aqueous layer was extracted with ethyl acetate, and the combined extracts washed with water and brine, dried (Na₂SO₄), filtered, and concentrated to provide 1.50 g (87%) of the desired product as a yellow oil that was used without further purification. MS (DCI/NH₃) m/z 671 (M+H)⁺; ¹H NMR (300 MHz, CDCl₃) 7.55 (d, 6H), 7.25 (t, 7H), 7.16 (m, 2H), 6.71 (s, 1H), 3.45 (br s, 2H), 3.01 (br s, 2H), 2.57 (br s, 2H), 1.52-0.89 (m, 27H).

### EXAMPLE 1E

### 1-cyclopropyl-4-oxo-7-(5-trityl-4,5,6,7-tetrahydrothieno[3,2-c]pyridin-2-yl)-1,4-dihydro-3-quinolinecarboxylic acid

Ethyl 7-bromo-1-cyclopropyl-4-oxo-1,4-dihydro-3-quinolone-3-carboxylate (0.104 g, 0.280 mmol), Example 1D (0.288 g, 0.430 mmol) and Pd(PPh₃)₂Cl₂ (0.020 g, 0.028 mmol) were combined in toluene (20 mL), heated at 90-110° C. for 24 hours, cooled, partitioned between ethyl acetate and 15% potassium fluoride, and filtered though diatomaceous earth (Celite®). The layers were separated, and the aqueous layer was extracted with ethyl acetate. The combined extracts were washed with brine, dried (Na₂SO₄), filtered, and concentrated. The residue was purified by flash flash column chromatography on silica gel with dichloromethane followed by 2% then 5% methanol in dichloromethane to provide 0.099 g (58%) of the desired product as an off-white solid. MS (DCI/NH₃) m/z 609 (M+H)⁺; ¹H NMR (300 MHz,CDCl₃) 8.85 (s, 1H), 8.44 (d, 1H), 8.13 (s, 1H), 7.76-7.70 (m, 2H), 7.53 (d, 5H), 7.26 (m, 6H), 7.17 (m, 2H), 7.12 (s, 1H), 3.58 (m, 1H), 3.46 (br s, 2H), 3.03 (br s, 2H), 2.65 (br s, 2H), 1.33 (m, 2H), 1.21 (m, 2H).

### EXAMPLE 1F

### 1-cyclopropyl-4-oxo-7-(4,5,6,7-tetrahydrothieno[3,2-c]pyridin-2-yl)-1,4-dihydro-3-quinolinecarboxylic acid hydrochloride

A solution of Example 1E (0.046 g, 0.076 mmol) in absolute ethanol (10 mL) and a minimal amount of chloroform at 0° C. was treated with 4M HCl in dioxane (76 mL, 0.304 mmol), stirred for 30 minutes, and diluted with ethyl ether (10 mL). The precipitate which formed was filtered and washed with ethyl ether to provide 0.015 g (49%) of the desired product as a yellow solid. MS (DCI/NH₃) m/z 367 (M+H)⁺; ¹H NMR (300 MHz, DMSO-d₆) 9.35 (s, 2H), 8.77 (s, 1H), 8.39 (d, 1H), 8.37 (m, 1H), 7.92 (d, 1H), 7.71 (s, 1H), 4.27 (br s, 2H), 3.93 (m, 1H), 3.47 (br s, 2H), 3.14 (br s, 2H), 1.34 (m, 2H), 1.24 (m, 2H).

### EXAMPLE 2

### 1-cyclopropyl-8-methoxy-4-oxo-7-(4,5,6,7-tetrahydrothieno[3,2-c]pyridin-2-yl)-1,4-dihydro-3-quinolinecarboxylic acid hydrochloride

### EXAMPLE 2A

### ethyl 1-cyclopropyl-8-methoxy-4-oxo-7-(5-trityl-4,5,6,7-tetrahydrothienor3 2-c]pyridin-2-yl)-1,4-dihydro-3-quinolinecarboxylate

Ethyl 7-bromo-1-cyclopropyl-8-methoxy-4-oxo-1,4-dihydro-3-quinolonecarboxylate and Example 1D were processed as described in Example 1E to provide the desired product. MS (DCI/NH₃) m/z 667 (M+H)⁺; ¹NMR (300 MHz, CDCl₃) 8.62 (s, 1H), 8.18 (d, 1H), 7.58 (m, 6H), 7.26 (m, 7H), 7.19 (m, 2H), 4.42 (q, 2H), 3.94 (m, 1H), 3.62 (s, 3H), 3.46 (br s, 2H), 3.05 (m, 2H), 2.65 (br s, 2H), 1.41 (t, 3H), 1.17 (m, 2H), 0.96 (m, 2H).

### EXAMPLE 2B

### 1-cyclopropyl-8-methoxy-4-oxo-7-(5-trityl-4,5,67-tetrahydrothieno[3,2-c]pyridin-2-yl)-1,4-dihydro-3-quinolinecarboxylic acid

A solution of Example 2A (0.590 g, 0.884 mmol) in 3:1/THF:water (30 mL) was treated with LiOH.H₂O (0.337 g, 8.21 mmol) and stirred overnight at room temperature. The reaction mixture was brought to pH 3.5-4.0 with 10% HCl, and extracted with ethyl acetate. The combined extracts were washed with brine, dried (Na₂SO₄), filtered, and concentrated to provide 0.553 g (80%) of the desired product which was used without further purification. MS (DCI/NH₃) m/z 639 (M+H)⁺; ¹H NMR (300 MHz, CDCl₃) 14.75 (s, 1H), 8.89 (s, 1H), 8.20 (d, 1H), 7.70 (d, 1H), 7.57 (d, 6H), 7.25 (m, 7H), 7.20 (m, 3H), 4.12 (m, 1H), 3.65 (s, 3H), 3.47 (s, 2H), 3.06 (m, 2H), 2.66 (br s, 2H), 1.24 (m, 2H), 1.05 (m, 2H).

### EXAMPLE 2C

### 1-cyclopropyl-8-methoxy-4-oxo-7-(4,5,6,7-tetrahydrothieno[3,2-]pyridin-2-yl)-1,4-dihydro-3-quinolinecarboxylic acid hydrochloride

Example 2B was processed as described in Example 1F to provide the desired product. mp 180° C. (decomp.); MS (DCI/NH₃) m/z 397 (M+H)⁺; ¹H NMR (300 MHz, DMSO-d₆) 9.70 (s, 2H), 8.80 (s, 1H), 8.15 (d, 1H), 7.98 (d, 1H), 7.71 (s, 1H), 4.27-4.22 (br m, 3H), 3.70 (s, 3H), 3.41 (br s, 2H), 3.16 (m, 2H), 1.16 (m, 2H), 1.05 (m, 2H).

### EXAMPLE 3

### 1-cyclopropyl-4-oxo-7-(4,5,6,7-tetrahydrothieno[3,2-c]pyridin-2-yl)-1,4-dihydro[1,8]naphthyridine-3-carboxylic acid hydrochloride

### EXAMPLE 3A

### 1-cyclopropyl-4-oxo-7-(5-trityl-4,5,6,7-tetrahydrothieno[3,2-c]pyridin-2-yl -1,4-dihydro[1,8]naphthyridine-3-carboxylic acid

Ethyl 1-cyclopropyl-7-chloro-4-oxo-1,4-dihydro[ 1,8]naphthyridine-3-carboxylate and Example 1D were processed as described in Example 1E to provide the desired product. MS (DCI/NH₃) m/z 610 (M+H)⁺; ¹H NMR (300 MHz, CDCl₃) 14.6 (s, 1H), 8.90 (s, 1H), 8.63 (d, 1H), 7.68 (d, 1H), 7.55 (m, 6H), 7.26 (m, 8H), 7.18 (m, 2H), 3.80 (m, 1H), 3.45 (br s, 2H), 3.05 (m, 2H), 2.66 (br s, 2H), 1.40 (m, 2H), 1.15 (m, 2H).

### EXAMPLE 3B

### 1-cyclopropyl-4-oxo-7-(4,5,6,7-tetrahydrothieno[3,2-c]pyridin-2-yl)-1,4-dihydro[1,8]naphthyridine-3-carboxylic acid hydrochloride

Example 3A was processed as described in Example 1F to provide the desired product. MS (DCI/NH₃) m/z 367 (M+H)⁺; ¹H NMR (500 MHz, CF₃CO₂D) 10.01 (s, 1H), 9.51 (d, 1H), 8.83 (d, 1H), 8.48 (s, 1H), 4.92 (m, 2H), 4.43 (m, 2H), 4.02 (m, 2H), 2.23 (m, 2H), 1.96 (m, 2H).

### EXAMPLE 4

### 1-cyclopropyl-6-fluoro-4-oxo-7-(4,5,6,7-tetrahydrothieno[3,2-c]pyridin-2-yl)-1,4-dihydro[1,8]naphthyridine-3-carboxylic acid hydrochloride

### EXAMPLE 4A

### ethyl 1-cyclopropyl-6-fluoro-4-oxo-7-(5-trityl-4,5,6,7-tetrahydrothieno[3,2-c]pyridin-2-yl)-1,4-dihydro[1,8]naphthyridine-3-carboxylate

Ethyl 1-cyclopropyl-7-chloro-6-fluoro-4-oxo-1,4-dihydro[1,8]naphthyridine-3-carboxylate and Example 1D were processed as described in Example 1E to provide the desired product. MS (APCI(+)) m/z 656 (M+H)+⁺; ¹H NMR (300 MHz, DMSO-d₆) 8.64 (s, 1H), 8.34 (d, 1H), 7.60-7.52 (m, 7H), 7.35-7.14 (m, 9H), 4.40 (q, 2H), 3.66 (m, 1H), 3.45 (br s, 2H), 3.07 (m, 2H), 2.65 (m, 2H), 1.41 (t, 3H), 1.36-1.15 (m, 2H), 1.08 (m, 2H).

### EXAMPLE 4B

### 1 -cyclopropyl-6-fluoro-4-oxo-7-(5-trityl-4,5,6,7-tetrahydrothieno[3,2-c]pyridin-2-yl)- 1,4-dihydro[1,8]naphthyridine-3-carboxylic acid

Example 4A was processed as described in Example 2B to provide the desired product. MS (APCI(+)) m/z 628 (M+H)+⁺; ¹H NMR (300 MHz, DMSO-d₆) 14.40 (s, 1H), 8.90 (s, 1H), 8.34 (d, 1H), 7.59-7.50 (m, 7H), 7.35-7.12 (m, 9H), 3.80 (m, 1H), 3.47 (m, 2H), 3.08 (m, 2H), 2.67 (m, 2H), 1.23 (m, 2H), 1.13 (m, 2H).

### EXAMPLE 4C

### 1-cyclopropyl-6-fluoro-4-oxo-7-(4,5,6,7-tetrahydrothieno[3,2-c]pyridin-2-yl)-1,4-dihydro[1,8]naphthyridine-3-carboxylic acid hydrochloride

Example 4B was processed as described in Example 1F to provide the desired product. MS (APCI(+)) m/z 386 (M+H)+⁺; ¹H NMR (300 MHz, DMSO-d6) 9.41 (br s, 2H), 8.82 (s, 1H), 8.57 (d, 1H), 7.91 (d, 1H), 4.29 (br s, 2H), 3.83 (m, 1H), 3.46 (m, 2H), 3.18 (m, 2H), 1.37-1.23 (m, 2H), 1.22-1.14 (m, 2H).

### EXAMPLE 5

### 1-cyclopropyl-8-(difluoromethoxy)-4-oxo-7-(4,5,6,7-tetrahydrothieno[3,2-c]pyridin-2-yl)-1,4-dihydro-3-quinolinecarboxylic acid hydrochloride

### EXAMPLE 5A

### ethyl 1-cyclopropyl-8-((difluoromethyl)peroxy)-4-oxo-7-(5-trityl-4,5,6,7-tetrahydrothieno[3,2-c]pyridin-2-yl)-1,4-dihydro-3-quinolinecarboxylate

Ethyl 1-cyclopropyl-7-bromo-8-difluoromethoxy-4-oxo-1,4-dihydroquinolone-3-carboxylate and Example 1D were processed as described in Example 1E to provide the desired product. MS (APCI(+)) m/z 703 (M+H)⁺; ¹H NMR (300 MHz, DMSO-d₆) 8.64 (s, 1H), 8.34 (d, 1H), 7.59-7.50 (m, 8H), 7.34-7.13 (m, 9H), 6.13 (t, 1H), 4.40 (q, 2H), 4.10 (m, 1H), 3.45 (m, 2H), 3.05 (m, 2H), 2.65 (m, 2H), 1.43 (t, 3H), 1.45-1.20 (m, 4H).

### EXAMPLE 5B

### 1-cyclopropyl-8-(difluoromethoxy)-4-oxo-7-(5-trityl-4,5,6,7-tetrahydrothieno[3,2-c]pyridn-2-yl)-1,4-dihydro-3-quinolinecarboxylic acid

Example 5A was processed as described in Example 2B to provide the desired product. MS (APCI(+)) m/z 675 (M+H)⁺; ¹H NMR (300 MHz, DMSO-d₆) 8.93 (s, 1H), 8.37 (d, 1H), 7.69 (d, 1H), 7.61-7.50 (m, 7H), 7.35-7.10 (m, 9H), 6.17 (t, 11H), 4.23 (m, 11H), 3.47 (m, 2H), 3.06 (m, 2H), 2.67 (m, 2H), 1.37-1.23 (m, 2H), 1.09-0.99 (m, 2H).

### EXAMPLE 5C

### 1-cyclopropyl-8-(difluoromethoxy)-4-oxo-7-(4,5,6,7-tetrahydrothieno[3,2-c]pvridin-2-yl)-1,4-dihydro-3-quinolinecarboxylic acid hydrochloride

Example 5B was processed as described in Example 1F to provide the desired product. MS (APCI(+)) m/z 433 (M+H)⁺; ¹H NMR (300 MHz, DMSO-d₆) 9.37 (br s, 2H), 8.87 (s, 1H), 8.31 (d, 1H), 7.95 (d, 1H), 7.64 (s, 1H), 7.00 (t, 1H), 4.26 (m, 1H), 4.14 (m, 2H), 3.46 (m, 2H), 3.14 (m, 2H), 1.18 (m, 2H), 1.04 (m, 2H).

### EXAMPLE 6

### 1-cycloproyl-4-oxo-7-(4,5,6,7-tetrhydrothieno(2,3-c)pyrdin-2-yl)-1,4-dihydro-3-quinolinecarboxylic acid hydrochloride

### EXAMPLE 6A

### N-methylene-2-(3-thienyl)ethanamine 2-(3-Thienyl)ethylamine was processed according to Example 1A to provide the desired product. MS (DCI/NH₃) m/z 140 (M+H)⁺ and 157 (M+NH₄)⁺; ¹H NMR (300 MHz, CDCl₃) 7.08 (d, 1H), 6.95 (d, 1H), 6.89 (d, 1H), 3.64 (m, 2H), 2.98 (dd, 2H), 2.78 (dd, 2H).

### EXAMPLE 6B

### 4,5,6,7-tetrahydrothieno(2,3-c)pyridine

Example 6A was processed as described in Example 1B to provide the desired product. MS (DCI/NH₃) m/Z 140 (M+H)⁺ and 157 (M+NH₄)⁺; ¹H NMR (300 MHz, CDCl₃) 7.09 (d, 1H), 6.77 (d, 1H), 3.82 (s, 2H), 2.93 (dd, 2H), 2.70 (m, 2H), 1.64 (br s, 1H).

### EXAMPLE 6C

### 6trityl-4,5,6,7-tetrahydrothieno(2,3-c)pyridine

Example 6B was processed as described in Example 1C to provide the desired product. MS (DCI/NH₃) m/z 382 (M+H)⁺; ¹H NMR (300 MHz, CDCl₃) 7.55 (d, 6H), 7.26 (m, 6H), 7.17 (m, 3H), 7.11 (d, 1H), 6.80 (d, 1H), 3.52 (br s, 2H), 2.84 (dd, 2H), 2.55 (m, 2H).

### EXAMPLE 6D

### 2-(tributylstannyl)-6-trityl-4,5,6,7-tetrahydrothieno(2,3-c)2yridine

Example 6C was processed as described in Example 1D to provide the desired product. MS (DCI/NH₃) m/z 575 (M+NH₄)⁺; ¹H NMR (300 MHz, CDCl₃) 7.55 (d, 6H), 7.25 (m, 6H), 7.17 (m, 3H), 6.83 (s, 1H), 3.55 (m, 2H), 2.84 (dd, 2H), 2.51 (m, 2H), 1.53-1.05 (m, 18H), 0.90 (t, 9H).

### EXAMPLE 6E

### 1-cyclopropyl-4-oxo-7-(6-trityl-4,5,6,7-tetrahydrothieno(2,3-c)pyridin-2-yl)-1,4-dihydro-3-quinolinecarboxylic acid

Ethyl 7-bromo-1-cyclopropyl-4-oxo-1,4-dihydro-3-quinolone-3-carboxylate and Example 6D were processed as described in Example 1E to provide the desired product. MS (DCI/NH₃) m/z 609 (M+H)⁺; 1H NMR (300 MHz, CDCl₃) 8.93 (s, 1H), 8.46 (d, 1H), 8.40 (d, 1H), 7.90 (dd, 1H), 7.60-7.20 (br m, 16H), 4.53 (s, 2H), 3.84 (m, 1H), 3.61 (dd, 2H), 3.12 (dd, 2H), 1.45 (m, 2H), 1.26 (m, 2H).

### EXAMPLE 6F

### 1 -cyclopropyl-4-oxo-7-(4,5,6,7-tetrahydrothieno(2,3-c)pyridin-2-yl)-1,4-dihydro-3-quinolinecarboxylic acid hydrochloride

Example 6E was processed as described in Example 1F to provide the desired product. mp >300° C.; MS (DCI/NH₃) m/z 367 (M+H)⁺; ¹H NMR (300 MHz, CD₃OD) 8.91 (s, 1H), 8.44 (d, 1H), 8.42 (d, 1H), 7.90 (dd, 1H), 7.60 (s, 1H), 4.51 (s, 2H), 3.84 (m, 1H), 3.59 (dd, 2H), 3.10 (dd, 2H), 1.45 (m, 2H), 1.26 (m, 2H).

### EXAMPLE 7

### 1-cyclopropyl-8-methoxy-4-oxo-7-(4,5,6,7-tetrahydrothieno(2,3-c)pyridin-2-yl)-1,4-dihydro-3-quinolinecarboxylic acid hydrochloride

### EXAMPLE 7A

### 1-cyclopropyl-8-methoxy-4-oxo-7-(6-trityl-4,5,6,7-tetrahydrothieno(2,3-c)pyridin-2-yl)-1,4-dihydro-3-quinolinecarboxylic acid

Ethyl 7-bromo-1-cyclopropyl-8-methoxy-4-oxo-1,4-dihydro-3-quinolonecarboxylate and Example 6D were processed as described in Example 1E to provide the desired product. MS (DCI/NH₃) m/z 639 (M+H)⁺; ¹H NMR (300 MHz, CDCl₃) 14.75 (s, 1H), 8.90 (s, 1H), 8.20 (d, 1H), 7.75 (d, 1H), 7.60-7.10 (br m, 16H), 4.10 (m, 1H), 3.65 (s, 3H), 3.57 (m, 2H), 2.90 (dd, 2H), 2.60 (m, 2H), 1.12 (m, 2H), 1.00 (m, 2H).

### EXAMPLE 7B

### 1-cyclopropyvl-8-methoxy-4-oxo-7-(4,5,6,7-tetrahydrothieno(2,3-c)pyridin-2-yl)-1,4-dihydro-3-quinolinecarboxylic acid hydrochloride

Example 7A was processed as described in Example 1F to provide the desired product. mp 188-189° C.; MS (DCI/NH₃) m/z 397 (M+H)⁺; ¹H NMR (300 MHz, DMSO-d₆) 9.40 (br s, 2H), 9.25 (br s, 1H), 8.80 (s, 1H), 8.13 (d, 1H), 8.02 (d, 1H), 7.71 (s, 1H), 4.45 (m, 2H), 4.26 (m, 1H), 3.70 (s, 3H), 3.40 (dd, 2H), 2.96 (dd, 2H), 1.15 (m, 2H), 1.08 (m, 2H).

### EXAMPLE 8

### 1-cyclopropyl-4-oxo-7-(4,5,6,7-tetrahydrothieno(2,3-c)pyridin-2-yl)-1,4-dihydro[1,8]naphthyridine-3-carboxylic acid hydrochloride

### EXAMPLE 8A

### 1-cyclopropyl-4-oxo-7-(6-trityl-4,5,6,7-tetrahydrothieno(2,3-c)pyridin-2-yl)-1,4-dihydro[1,8]naphthyridine-3-carboxylic acid

Ethyl 1-cyclopropyl-7-chloro-4-oxo-1,4-dihydro[1,8]naphthyridine-3-carboxylate and Example 6D were processed as described in Example 1E to provide the desired product. MS (DCI/NH₃) m/z 610 (M+H)⁺; ¹H NMR (300 MHz, CDCl₃) 8.90 (s, 1H), 8.73 (d, 1H), 8.24 (d, 1H), 8.00 (s, 1H), 7.60-7.10 (m, 15H), 4.52 (s, 2H), 3.90 (m, 1H), 3.41 (m, 2H), 2.97 (dd, 2H), 1.28 (m, 2H), 1.15 (m, 2H).

### EXAMPLE 8B

### 1-cyclopropyl-4-oxo-7-(4,5,6,7-tetrahydrothieno(2,3-c)pyridin-2-yl)-1,4-dihydro[1,8]naphthyridine-3-carboxylic acid hydrochloride

Example 8A was processed as described in Example 1F to provide the desired product. mp 298-300° C.; MS (DCI/NH₃) m/z 368 (M+H)⁺; ¹H NMR (300 MHz, DMSO-d₆) 9.65 (br s, 2H), 9.50 (br s, 1H), 8.80 (s, 1H), 8.68 (d, 1H), 8.17 (d, 1H), 8.02 (s, 1H), 4.45 (s, 2H), 3.83 (m, 1H), 3.40 (dd, 2H), 2.95 (dd, 2H), 1.28 (m, 2H), 1.15 (m, 2H).

### EXAMPLE 9

### 1-cyclopropyl-6-fluoro-4-oxo-7-(4,5,6,7-tetrahydrothieno(2,3-c)pyridin-2-yl)-1,4-dihydro[1,8]naphthyridine-3-carboxylic acid hydrochloride

### EXAMPLE 9A

### 1-cycloprgpyl-6-fluoro-4-oxo-7-(6-triyl-4,5,6,7-tetrahydrothieno(2,3-c,pyridin-2-yl)-1,4-dihydro[1,8]naphthyridine-3-carboxylic acid

Ethyl 1-cyclopropyl-7-chloro-6-fluoro-4-oxo-1,4-dihydro[1,8]naphthyridine-3-carboxylate and Example 6D were processed as described in Example 1E to provide the desired product. MS (DCI/NH₃) m/z 628 (M+H)⁺; ¹H NMR (300 MHz, CDCl₃) 14.40 (s, 1H), 8.90 (s, 1H), 8.65 (d, 1H), 8.30 (d, 1H), 7.60-7.10 (m, 15H), 3.83 (m, 1H), 3.60 (m, 2H), 2.90 (m, 2H), 2.62 (m, 2H), 1.32 (m, 2H), 1.25 (m, 2H).

### EXAMPLE 9B

### 1-cyclopropyl-6-fluoro-4-oxo-7-(4,5,6,7-tetrahydrothieno(2,3-c)pyridin-2-yl)-1,4-dihydro[1,8]naphthyridine-3-carboxylic acid hydrochloride

Example 9A was processed as described in Example 1F to provide the desired product. mp 290-291° C.; MS (DCI/NH₃) m/z 386 (M+H)⁺; ¹H NMR (300 MHz, DMSO-d₆) 9.30 (br s, 2H), 9.10 (br s, 11H), 8.82 (s, 1H), 8.57 (d, 11H), 7.91 (d, 1H), 4.50 (br s, 2H), 3.83 (m, 1H), 3.45 (dd, 2H), 3.00 (dd, 2H), 1.32 (m, 2H), 1.25 (m, 2H).

### EXAMPLE 10

### 1-cylopropyl-8-(difluoromethoxvy-4-oxo-7-(4,5,6,7-tetrahydrothieno(2,3-c)pyridin-2-yl-1,4-dihydro-3-quinolinecarboxlic acid hydrochloride

### EXAMPLE 10A

### ethyl 1-cyclopropyl-8-(difluoromethoxy)-4-oxo-7-(6-trityl-4,5,6,7-tetrahydrothieno(2,3-c)pyridin-2-yl)-1,4-dihydro-3-quinolinecarboxylate

Ethyl 1-cyclopropyl-7-bromo-8-difluoromethoxy-4-oxo-1,4-dihydroquinolone-3-carboxylate and Example 6D were processed as described in Example 1E to provide the desired product. MS (DCI/NH₃) m/z 703 (M+H)⁺; ¹H NMR (300 MHz, CDCl₃) 8.90 (s, 1H), 8.60(d, 1H), 8.37 (d, 1H), 7.70 (d, 1H), 7.60-7.10 (m, 16H), 6.18 (dd, 1H), 4.40 (q, 2H), 4.23 (m, 1H), 3.60 (m, 2H), 2.90 (dd, 2H), 2.62 (m, 2H), 1.20 (m, 2H), 1.05 (m, 2H), 0.93 (t, 3H).

### EXAMPLE 10B

### 1-cyclopropyl-8-(difluoromethoxy)-4-oxo-7-(6-trityl-4,5,6,7-tetrahydrothieno(2,3-c)pyridin-2-yl)-1,4-dihydro-3-quinolinecarboxylic acid

Example 10A was processed as described in Example 2B to provide the desired product. MS (DCI/NH₃) m/z 675 (M+H)⁺; ¹H NMR (300 MHz, CDCl₃) 8.90 (s, 1H), 8.47 (d, 1H), 7.70 (d, 1H), 7.60-7.10 (br m, 16H), 6.27 (dd, 1H), 4.23 (m, 1H), 3.60 (m, 2H), 2.90 (dd, 2H), 2.63 (m, 2H), 1.20 (m, 2H), 1.08 (m, 2H).

### EXAMPLE 10C

### 1-cyclopropyl-8-(difluoromethoxy)-4-oxo-7-(4,5,6,7-tetrahydrothieno(2,3-c)pyridin-2-yl -1,4-dihydro-3-quinolinecarboxylic acid hydrochloride

Example 10B was processed as described in Example 1F to provide the desired product. mp 198-200° C.; MS (APCI(+)) m/z 433 (M+H)⁺; ¹H NMR (300 MHz, DMSO-d₆) 9.48 (br s, 2H), 9.35 (br s, 1H), 8.88 (s, 1H), 8.30 (d, 1H), 7.98 (d, 1H), 7.65 (s, 1H), 7.01 (dd, 1H), 4.44 (br s, 2H), 4.35 (dd, 2H), 4.13 (m, 1H), 3.42 (dd, 2H), 3.08 (dd, 2H), 1.20 (dd, 2H), 1.05 (m, 2H).

### EXAMPLE 11

### 1-cyclopropyl-7-(6-methyl-4,5,6,7-tetrahydrothieno[3,2-c]pyridin-2-yl)-4-oxo-1,4-dihydro[1,8]naphthyridine-3-carboxylic acid hydrobromide

### EXAMPLE 11A

### N-methylene-1-(2-thienyl)-2-propanamine

2-(2-Aminopropyl)-thiophene (prepared by the method of Gilsdorf, et. al. J. Org. Chem. 1950, 15, 807) was processed as described in Example 1A to provide the desired product. MS (DCI/NH₃) m/Z 154 (M+H)⁺; ¹H NMR (300 MHz, CDCl₃) 7.13 (d, 1H), 6.91 (m, 1H), 6.79 (d, 1H), 3.70 (s, 2H), 3.17-3.07 (m, 2H), 2.80-2.73 (m, 1H), 1.05 (d, 3H).

### EXAMPLE 11B

### 6-methyl-4,5,6,7-tetrahydrothieno[3,2-c]pyridine

Example 11A was processed as described in Example 1B to provide the desired product. MS (DCI/NH₃) m/z 154 (M+H)⁺; ¹H NMR (300 MHz, CDCl₃) 7.06 (d, 1H), 6.73 (d, 1H), 3.75-3.68 (m, 1H), 3.03-2.97 (m, 1H), 2.78-2.74 (m, 1H), 2.56-2.44 (m, 2H), 1.06 (d, 3H).

### EXAMPLE 11C

### benzyl 6-methyl-6,7-dihydrothieno[3,2-c]pyridine-5(4H)-carboxylate

A solution of Example 11B (2.60 g, 16.8 mmol) in THF (20 mL) at 0° C. was treated with triethylamine (2.37 g, 23.5 mmol) and benzyl chloroformate (3.16 g, 18.5 mmol), stirred for 4 hours, and partitioned between water and ethyl acetate. The aqueous phase was extracted with ethyl acetate, the combined extracts were washed with water and brine, dried (Na₂SO₄), filtered, and concentrated. The residue was purified by flash column chromatography on silica gel with 10% then 20% ethyl acetate in hexanes to provide 1.50 g (34%) of the desired product. MS (DCI/NH₃) m/z 305 (M+NH₃)⁺; ¹H NMR (500 MHz, CDCl₃) 7.37 (m, 5H), 7.1 (d, 1H), 6.77 (d, 1H), 5.13 (s, 2H), 4.94-4.90 (br s, 2H), 4.15 (m, 1H), 3.08 (m, 11H), 2.63 (m, 11H), 1.15 (d, 1H).

### EXAMPLE 11D

### benzyl 6-methyl-2-(tributylstannyl)-6,7-dihydrothieno[3,2-c]pyridine-5(4H)-carboxylate

A solution of diisopropylamine (0.47 mL, 3.36 mmol) in THF (10 mL) at 0° C. was treated dropwise with 2.5M n-butyllithium in hexanes (1.3 mL, 3.36 mmol), cooled to −78° C., and stirred for 1 hour. This solution was added dropwise via canulae to a solution of Example 11C (0.80 g, 2.80 mmol) in THF (10 mL) at −78 ° C., stirred for 1 hour at −78° C., treated with chlorotributylstannane (0.96 g, 2.90 mmol), and warmed to room temperature overnight. The reaction mixture was partitioned between water and ethyl acetate. The aqueous phase was extracted with ethyl acetate, the combined extracts were washed with water and brine, dried (Na₂SO₄), filtered, and concentrated to provide 1.50 g (90%) of the desired product as a yellow oil. MS (APCI(+)) m/z 577 (M+H)⁺; ¹H NMR (300 MHz, CDCl₃) 7.38 (m, 5H), 6.82 (s, 11H), 5.17 (s, 2H), 4.21-4.16 (m, 2H), 3.14-3.09 (m, 1H), 2.72-2.62 (m, 2H), 1.56-0.98 (m, 30H).

### EXAMPLE 11E

### ethyl 7-(5-((benzyloxycarbonyl)-6-methyl-4,5,6,7-tetrahydrothieno[3,2-c]pyridin-2-yl)-1-cyclopropyl-4-oxo-1,4-dihydro[1,8]naphthyridine-3-carboxylate

Ethyl 1-cyclopropyl-7-chloro-4-oxo-1,4-dihydro[1,8]naphthyridine-3-carboxylate and Example 11D were processed as described in Example 1E to provide the desired product. MS (DCI/NH₃) m/z 544 (M+H)⁺; ¹H NMR (300 MHz, CDCl₃) 8.61 (s, 1H), 8.59 (d, 1H), 7.49 (m, 5H), 7.38 (d, 1H), 7.19 (s, 1H), 5.13 (s, 2H), 4.93-4.88 (br s, 2H), 4.32 (q, 2H), 4.16 (s, 1H), 4.10 (s, 1H), 3.60 (m, 1H), 3.12 (m, 1H), 2.67 (s, 1H), 2.61 (s, 1H), 1.61 (br s, 2H), 1.35 (t, 3H), 1.25 (m, 2H), 1.22 (d, 3H), 0.98 (m, 2H).

### EXAMPLE 11F

### 7-(5-((benzyloxy)carbonyl)-6-methyl-4,5,6,7-tetrahydrothieno[3,2-c]pyridin-2-yl)-1-cyclopropyl-4-oxo-1,4-dihydro[1,8]naphthyridine-3 -carboxylic acid

Example 11E was processed as described in Example 2B to provide the desired product. MS (DCI/NH₃) m/z 516 (M+H)⁺; ¹H NMR (300 MHz, CDCl₃) 14.5 (s, 11H), 8.92 (s, 1H), 8.71 (d, 11H), 7.7 (d, 11H), 7.37 (m, 5H), 6.98 (s, 1H), 5.20 (s, 2H), 5.02-4.96 (m, 2H), 4.24-4.19 (m, 1H), 3.80 (m, 1H), 3.18 (m, 1H), 2.76-2.73 (m, 1H), 1.22 (d, 3H), 1.15 (m, 2H), 0.89 (m, 2H).

### EXAMPLE 11G

### 1-cyclopropyl-7-(6-methyl-4,5,6,7-tetrahydrothieno[3,2-clpvridin-2-yl)-4-oxo-1,4-dihydro[1,8]naphthyridine-3-carboxylic acid hydrobromide

A solution of Example 11F (0.038 g, 0.075 mmol) in acetic acid (10 mL) at 0° C., treated with 30% HBr (0.202 mL) in acetic acid, warmed to room temperature, and stirred for 2 hours, and concentrated. The concentrate was triturated in diethyl ether, filtered, and washed sequentially with diethyl ether, hexanes, and dichloromethane to provide 0.020 g (59%) of the desired product. MS (APCI(+)) m/z 445 (M+2Na+NH₄)⁺; ¹H NMR (300 MHz, DMSO-d₆) 9.22 (s, 11H), 9.05 (s, 11H), 8.81 (s, 11H), 8.71 (d, 1H ), 8.15 (d, 11H), 7.98 (s, 1H), 4.35-4.32 (br s, 2H), 3.81 (m, 1H), 3.48-3.25 (m, 1H), 2.94-2.82 (m, 2H), 1.41 (d, 3H), 1.28 (m, 2H), 1.17 (m, 2H).

### EXAMPLE 12

### 1-cyclopropyl-6-fluoro-7-(6-methyl-4,5,6,7-tetrahydrothieno[3,2-c]pyridin-2-yl)-4-oxo-1,4-dihydro[1,8]naphthyridine-3-carboxylic acid hydrobromide

### EXAMPLE 12A

### ethyl 7-(5-((benzaloxycarbonyl)-6-methyl-4,5,6,7-tetrahydrothieno[3,2-c]pyridin-2-yl)-1-cyclopropyl-6-fluoro-4-oxo-1,4-dihydro[1,8]naphthyridine-3-carboxylate

Ethyl 1-cyclopropyl-7-chloro-6-fluoro-4-oxo- 1,4-dihydro[1,8]naphthyridine-3-carboxylate and Example 11D were processed as described in Example 1E to provide the desired product. MS (APCI(+)) m/z 562 (M+H)⁺; ¹H NMR (300 MHz, CDCl₃) 8.66 (s, 1H), 8.38 (d, 1H), 7.38 (m, 6H), 5.20 (s, 2H), 5.02-4.96 (m, 2H), 4.42 (q, 2H), 4.25 (s, 1H), 4.19 (s, 1H), 3.67 (m, 1H), 3.15 (m, 1H), 2.76 (s, 1H), 2.71 (s, 1H), 1.41 (t, 3H), 1.33 (m, 2H), 1.21 (d, 3H), 1.09 (m, 2H).

### EXAMPLE 12B

### 7-(5-((benzyloxy)carbonyl-6-methyl-4,5,6,7-tetrahydrothieno[3,2-c]pyridin-2-yl)-1-cyclopropyl-6-fluoro-4-oxo-1,4-dihydro[1,8]naphthyridine-3-carboxylic acid

Example 12A was processed as described in Example 2B to provide the desired product. MS (APCI(31 )) m/z 568 (M+Cl)⁺; ¹NMR (300 MHz, CDCl₃) 14.33 (s, 1H), 8.92 (s, 1H), 8.40 (d, 1H), 7.36 (m, 6H), 5.20 (s, 2H), 5.02-4.98 (m, 2H), 4.25-4.20 (m, 1H), 3.82 (m, 1H), 3.16 (m, 1H), 2.78-2.73 (m, 1H,), 1.42 (m, 2H), 1.22 (d, 3H), 1.13 (m, 2H).

### EXAMPLE 12C

### 1-cyclopropyl-6-fluoro-7-(6-methyl-4,5,6,7-tetrahydrothieno[3,2-c]pyridin-2-yl)-4-oxo-1,4-dihydro[1,8]naphthyridine-3-carboxylic acid hydrobromide

Example 12B was processed as described in Example 11G to provide the desired product. MS (APCI(+)) m/z 400 (M+H)⁺; ¹H NMR (300 MHz, DMSO-d₆) 9.39 (br s, 1H), 9.15 (br s, 1H), 8.81 (s, 1H), 8.57 (d, 11H), 7.92 (s, 1H), 4.43-4.32 (m, 3H), 3.83 (m, 1H), 3.30 (m, 1H), 2.97-2.91 (m, 1H), 1.42 (d, 3H), 1.29 (m, 2H), 1.18 (m, 2H).

### EXAMPLE 13

### 1-cyclopropyl-7-(6-methyl-4,5,6,7-tetrahydrothieno[3,2-c]pyridin-2-yl)-4-oxo-1,4-dihydro-3-quinolinecarboxylic acid hydrobromide

### EXAMPLE 13A

### 7-(5-((benzyloxy)carbonyl)-6-methyl-4,5,6,7-tetrahydrothieno[3,2-c]pyridin-2-yl)-1-cyclopropyl-4-oxo-1,4-dihydro-3-quinolinecarboxylic acid

Ethyl 1-cyclopropyl-7-bromo-4-oxo-1,4-dihydroquinolone-3-carboxylate and Example 11D were processed as described in Example 1E to provide the desired product. MS (APCI(−)) m/z 549 (M+C )⁺; ¹H NMR (300 MHz, DMSO-d₆) 15.0 (s, 1H), 8.75 (s, 1H), 8.37 (s, 1H), 8.36 (d, 1H), 7.89 (d, 1H), 7.39 (m, 6H), 5.15 (s, 2H), 4.91-4.85 (m, 2H), 4.20 (m, 1H), 3.91 (m, 1H), 2.82 (s, 1H), 2.76 (s, 1H), 1.34 (m, 2H), 1.26 (m, 2H), 1.18 (d, 3H).

### EXAMPLE 13B

### 1-cyclopropyl-7-(6-methyl-4,5,6,7-tetrahydrothieno[3,2-c]pyridin-2-yl)-4-oxo-1,4-dihydro-3-quinolinecarboxylic acid hydrobromide

Example 13A was processed as described in Example 11G to provide the desired product. MS (APCI(+)) m/z 381 (M+H)⁺; ¹H NMR (300 MHz, DMSO-d₆) 9.22 (br s, 1H), 9.07 (br s, 1H), 8.77 (s, 1H), 8.39 (d, 1H), 8.35 (s, 1H), 7.92 (d, 1H), 7.72 (s, 1H), 4.33-4.25 (br s, 3H), 3.93 (m, 1H), 3.27 (m, 1H), 2.93-2.84 (m, 1H), 1.42 (d, 3H), 1.36 (m, 2H), 1.23 (m, 2H).

### EXAMPLE 14

### 1-cyclopropyl-8-(difluoromethoxy)-7-(6-methyl-4,5,6,7-tetrahydrothieno[3,2-c]pyridin-2-yl-4-oxo-1,4-dihydro-3-quinolinecarboxylic acid hydrobromide

### EXAMPLE 14A

### ethyl 7-(5-((benzyloxycarbonyl)-6-methyl-4,5,6,7-tetrahydrothieno[3,2-c]pyridin-2-1-cyclopropyl-8-(difluoromethoxy)-4-oxo-1,4-dihydro-3-quinolinecarboxylate

Ethyl 1-cyclopropyl-7-bromo-8-difluoromethoxy-4-oxo-1,4-dihydroquinolone-3-carboxylate and Example 11D were processed as described in Example 1E to provide the desired product. MS (APCI(−)) m/z 643 (M+C1)⁺; ¹H NMR (300 MHz, CDCl₃) 8.65 (s, 1H), 8.37 (d, 1H), 7.68 (d, 1H), 7.34 (m, 6H), 6.14 (dd, 1H), 5.20 (s, 2H), 4.41 (q, 2H), 4.10 (m, 1H), 3.18 (br s, 1H), 2.73 (s, 1H), 2.68 (s, 1H), 1.41 (t, 3H), 1.27 (m, 2H), 0.98 (m, 2H).

### EXAMPLE 14B

### 7-(5-((benzyloxy)carbonyl)-6-methyl-4,5,6,7-tetrahydrothieno[3,2-c]pyridin-2-yl)-1-cyclopropyl-8-(difluoromethoxy)-4-oxo-1,4-dihydro-3-quinolinecarboxylic acid

Example 14A was processed as described in Example 2B to provide the desired product. MS (APCI(+)) m/z 581 (M+H)⁺; ¹H NMR (300 MHz, CDCl₃) 8.94 (s, 1H), 8.39 (d, 1H), 7.70 (d, 1H), 7.39 (m, 6H), 6.18 (dd, 1H), 5.20 (s, 2H), 5.03-4.98 (br s, 2H), 4.25 (m, 1H), 3.18 (m, 1H), 2.75 (s, 1H), 2.69 (s, 1H), 1.35 (m, 2H), 1.23 (d, 3H), 1.03 (m, 2H).

### EXAMPLE 14C

### 1-cyclopropyl-8-(difluoromethoxy)-7-(6-methyl-4,5,6,7-tetrahydrothieno[3,2-c]pyridin-2-yl)-4-oxo-1-dihydro-3-quinolinecarboxylic acid hydrobromide

Example 14B was processed as described in Example 11G to provide the desired product. mp 210 ° C. (decomp.); MS (APCI(31 )) m/z 481 (M+Cl)⁺; ¹H NMR (300 MHz, DMSO-d₆) 9.25 (br s, 1H), 9.07 (br s, 1H), 8.87 (s, 1H), 8.30 (d, 1H), 7.94 (d, 1H), 7.65 (s, 1H), 7.01 (dd, 1H), 4.36-4.25 (m, 2H), 4.14 (m, 1H), 3.24 (m, 1H), 2.92-2.84 (m, 2H), 1.41 (d, 3H), 1.20 (m, 2H), 1.05 (m, 2H).

### EXAMPLE 15

### 1-cyclopropyl-8-methoxy-7-(6-methyl-4,5,6,7-tetrahydrothieno[3,2-c]pyridin-2-yl)-4-oxo-1,4-dihydro-3-quinolinecarboxylic acid hydrobromide

### EXAMPLE 15A

### 7-(5-((benzyloxycarbonyl)-6-methyl-4,5,6,7-tetrahydrothieno[3,2-c]pyridin-2-yl)-1-cyclopropyl-8-methoxy-4-oxo-1,4-dihydro-3-quinolinecarboxlic acid

Ethyl 1-cyclopropyl-7-bromo-8-methoxy-4-oxo-1,4-dihydroquinolone-3-carboxylate and Example 11D were processed as described in Example 1E to provide the desired product. MS (APCI(+)) m/z 545 (M+H)⁺; ¹H NMR (300 MHz, DMSO-d₆) 10.0 (s, 1H), 8.68 (s, 1H), 8.03 (d, 1H), 7.76 (d, 1H), 7.62 (s, 1H), 7.40 (m, 5H), 5.15 (s, 2H), 4.89-4.80 (m, 2H), 4.22 (br s, 1H), 4.09 (m, 1H), 3.65 (s, 3H), 3.09 (m, 1H), 2.79 (s, 1H), 2.74 (s, 1H), 1.15-1.12 (m, 5H), 0.86 (m, 2H).

### EXAMPLE 15B

### 1-cyclopropyl-8-methoxy-7-(6-methyl-4,5,6,7-tetrahydrothieno[3,2-c]pyridin-2-yl)-4-oxo-1,4-dihydro-3-quinolinecarboxylic acid hydrobromide

Example 15A was processed as described in Example 11G to provide the desired product. MS (APCI(+)) m/z 411 (M+H)⁺; ¹H NMR (500 MHz, DMSO-d₆) 8.81 (s, 1H), 8.15 (d, 1H), 7.98 (d, 1H), 7.71 (s, 1H), 4.35-4.26 (m, 4H), 3.69 (s, 3H), 3.27-3.23 (m, 1H), 2.92-2.87 (m, 1H), 1.42 (d, 3H), 1.16 (m, 2H), 1.05 (m, 2H).

### EXAMPLE 16

### 1-cyclopropyl-7-(4-methyl-4,5,6,7-tetrahydrothieno[3 2-c]pyridin-2-yl)-4-oxo-1,4-dihydro-3-quinolinecarboxylic acid hydrobromide

### EXAMPLE 16A

### benzyl 4-methyl-6,7-dihydrothieno[3,2-c]pyridine-5(4H)-carboxylate

4-Methyl-4,5,6,7-tetrahydrothieno[3,2-c]pyridine (prepared by the method of Descamps, et. al., Binon Bull. Soc. Chim. Belg. 1962, 71, 599) was processed as described in Example 11C to provide the desired product. MS (APCI(+)) m/z 288 (M+H)⁺; ¹H NMR (300 MHz, CDCl₃) 7.38-7.26 (m, 5H), 7.10 (dd, 11H), 6.77 (m, 1H), 5.18 (m, 2H), 4.43 (m, 1H), 3.72 -2.32 (m, 4H), 1.41 (d, 3H).

### EXAMPLE 16B

### benzyl 4-methyl-2-(tributylstannyl)-6,7-dihydrothieno[3,2-c]pyridine-5(4H)-carboxylate

Example 16A was processed according to Example 11D to provide the desired product. MS (APCI(+)) m/z 577 (M+H)⁺; ¹H NMR (300 MHz, CDCl₃) 7.38-7.26 (m, 5H), 7.10 (s, 1H), 5.18 (m, 2H), 4.45 (m, 11), 3.20-2.70 (m, 4H), 1.66-0.87 (m, 30H).

### EXAMPLE 16C

### 7-(5-((benzyloxy)carbonyl)-4-methyl-4,5,6,7-tetrahydrothieno[3,2-c]pyridin-2-yl)-1-cyclopropyl-4-oxo-1,4-dihydro-3 -quinolinecarboxylic acid

Ethyl 7-bromo-1-cyclopropyl-4-oxo-1,4-dihydro-3-quinolone-3-carboxylate and Example 16B were processed as described in Example 1E to provide the desired product. MS (DCI/NH₃) m/z 609 (M+H)⁺; ¹H NMR (300 MHz, DMSO-d₆) 8.75 (s, 1H), 8.37 (d, 1H), 8.33 (d, 1H), 7.91 (dd, 1H), 7.75 (s, 1H), 7.66-7.32 (m, 5H), 5.16 (m, 2H), 4.28 (m, 1H), 3.91 (m, 1H), 3.25 (m, 2H), 3.14 (m, 2H), 1.45-1.15 (m, 4H), 1.44 (d, 3H).

### EXAMPLE 16D

### 1-cyclopropyl-7-(4-methyl-4,5,6,7-tetrahydrothieno[3,2-c]pyridin-2-yl)-4-oxo-1,4-dihydro-3-quinolinecarboxylic acid hydrobromide

Example 16C was processed as described in Example 11G to provide the desired product. mp 203-207° C. (decomp.); MS (APCI(+)) m/z 381 (M+H)⁺; ¹H NMR (300 MHz, DMSO-d₆) 8.76 (s, 1H), 8.39 (d, 1H), 8.36 (d, 1H), 7.96 (dd, 1H), 7.84 (s, 1H), 4.59 (m, 1H), 3.94 (m, 1H), 3.61 (m, 2H), 3.14 (m, 2H), 1.64 (d, 3H), 1.39-1.19 (m, 4H).

### EXAMPLE 17

### 1-cyclopropyl-7-(4-methyl-4,5,6,7-tetrahydrothieno[3 2-c]pyridin-2-yl)-4-oxo-14-dihydro[1.8]naphthyridine-3-carboxylic acid hydrochloride

### EXAMPLE 17A

### 7-(5-((benzyloxy)carbonyl)-4-methyl-4,5,6,7-tetrahydrothieno[3,2-c]pyridin-2-yl)-1-cyclopropyl-4-oxo-1,4-dihydro[1,8]naphthyridine-3 -carboxylic acid

Ethyl 1-cyclopropyl-7-chloro-4-oxo-1,4-dihydro [1,8]naphthyridine-3-carboxylate and Example 16B were processed as described in Example 1E to provide the desired product as a mixture of interconverting conformational isomers. MS (APCI(−)) m/z 550 (M+Cl)⁻; ¹H NMR (300 MHz, CDCl₃) 8.93 (s, 0.2H), 8.92 (s, 0.8H), 8.72 (d, 0.2H), 8.70 (d, 0.8H), 7.77 (d, 1H), 7.70-7.35 (m, 6H), 5.20 (m, 2H), 4.40 (m, 1H), 3.82 (m, 1H), 3.15 (m, 2H), 2.85 (m, 2H), 1.48 (d, 3H), 1.54-1.12 (m, 4H).

### EXAMPLE 17B

### 1-cyclopropyl-7-(4-methyl-4,5,6,7-tetrahydrothieno[3,2-c]pyridin-2-yl)-4-oxo-1,4-dihydro[1,8]naphthyridine-3-carboxylic acid hydrochloride

A solution of Example 17A (0.170 g, 0.31 mmol) in trifluoroacetic acid (2 mL) was stirred for 16 hours, concentrated, treated with 4M HCl in dioxane (1 mL), stirred for 30 minutes, concentrated, triturated in diethyl ether, filtered, and washed with diethyl ether to provide 0.047 g (35%) of the desired product as a tan solid. mp 243-245° C. (decomp.); MS (APCI(+)) m/z 382 (M+H)⁺; ¹H NMR (300 MHz, DMSO-d₆) 8.81 (s, 1H), 8.72 (d, 1H), 8.20 (d, 1H), 8.13 (s, 1H), 4.57 (m, 1H), 3.85 -3.81 (m, 1H), 3.58 (m, 2H), 3.15 (m, 2H), 1.63 (d, 3H), 1.35-1.17 (m, 4H).

### EXAMPLE 18

### 1-cyclopropyl-6-fluoro-7-(4-methyl-4,5,6,7-tetrahydrothieno[3,2-c]pyridin-2-yl)-4-oxo-1,4-dihydro[1,8]naphthyridine-3-carboxylic acid hydrochloride

### EXAMAPLE 18A

### 7-(5-((benzyloxy)carbonyl)-4-methyl-4,5,6,7-tetrahydrothieno[3,2-c]pyridin-2-yl)-1-cyclopropyl-6-fluoro-4-oxo-1,4-dihydro[1,8]naphthyridine-3-carboxylic acid

Ethyl 1-cyclopropyl-7-chloro-6-fluoro-4-oxo-1,4-dihydro[1,8]naphthyridine-3-carboxylate and Example 16B were processed as described in Example 1E to provide the desired product. MS (APCI(+)) m/z 534 (M+H)⁺; ¹H NMR (300 MHz, DMSO-d₆) 8.81 (s, 1H), 8.53 (d, 1H), 7.94 (d, 1H), 7.66-7.33 (m, 5H), 5.15 (s, 2H), 4.30 (m, 1H), 3.83 (m, 1H), 3.20 (m, 2H), 2.94 (m, 2H), 1.43 (d, 3H), 1.35-1.17 (m, 4H).

### EXAMPLE 18B

### 1-cyclopropyl-6-fluoro-7-(4-methyl-4,5,6,7-tetrahydrothieno[3,2-c]pyridin-2-yl)-4-oxo-1,4-dihydro[1,8]naphthyridine-3-carboxylic acid hydrochloride

Example 18A was processed as described in Example 17B to provide the desired product. MS (APCI(+)) m/z 400 (M+H)⁺; ¹H NMR (300 MHz, DMSO-d₆) 8.83 (s, 1H), 8.58 (d, 1H), 7.94 (d, 1H), 4.61 (m, 1H), 3.84 (m, 1H), 3.40 (m, 2H), 3.18 (m, 2H), 1.63 (d, 3H), 1.36-1.17 (m, 4H).

### EXAMPLE 19

### 1-cyclopropyl-8-methoxy-7-(4-methyl-4,5,6,7-tetrahydrothieno[3,2-c]pyridin-2-yl)-4-oxo-1,4-dihydro-3-quinolinecarboxylic acid hydrobromide

### EXAMPLE 19A

### 7-(5-((benzyloxy)carbonyl)-4-methyl-4,5,6,7-tetrahydrothieno[3,2-c]pyridin-2-yl)-1-cyclopropyl-8-methoxy-4-oxo-1,4-dihydro-3-quinolinecarboxylic acid

Ethyl 7-bromo-1-cyclopropyl-8-methoxy-4-oxo- 1,4-dihydro-3-quinolonecarboxylate and Example 16B were processed as described in Example 1E to provide the desired product as an inseparable mixture of interconverting rotational isomers. MS (ESI(−)) m/z 543 (M-H)⁻; ¹H NMR (300 MHz, DMSO-d₆) 14.91 (s, 0.8H), 14.67 (s, 0.2), 8.81 (s, 0.8H), 8.79 (s, 0.2H), 8.12 (d, 0.8H), 8.06 (d, 0.2H), 8.02 (d, 0.8H), 7.82 (d, 0.2H), 7.76 (s, 1H), 7.66-7.31 (m, 5H), 5.16 (m, 2H), 4.60 (m, 1H), 4.25 (m, 1H), 3.69 (s, 3H), 3.21 (m, 2H), 2.86 (m, 2H), 1.44 (d, 3H), 1.16-1.03 (m, 4H).

### EXAMPLE 19B

### 1-cyclopropyl-8-methoxy-7-(4-methyl-4,5,6,7-tetrahydrothieno[3,2-c]pyridin-2-yl-4-oxo-1,4-dihydro-3-quinolinecarboxylic acid hydrobromide

Example 19A was processed as described in Example 11G to provide the desired product. mp 202-205° C. (decomp.); MS (APCI(+)) m/z 411 (M+H)⁺; ¹H NMR (300 MHz, DMSO-d₆) 8.81 (s, 1H), 8.15 (d, 1H), 8.08 (d, 1H), 7.84 (s, 1H), 4.58 (m, 1H), 4.26 (m, 1H), 3.70 (s, 3H), 3.21 (m, 2H), 3.13 (m, 2H), 1.63 (d, 3H), 1.03-1.16 (m, 4H).

### EXAMPLE 20

### 1-cyclopropyl-8-(difluoromethoxy)-7-(4-methyl-4,5,6,7-tetrahydrothieno[3,2-c]pyridin-2-yl)-4-oxo-1,4-dihydro-3-quinolinecarboxylic acid hydrobromide

### EXAMPLE 20A

### 7-(5-((benzyloxy)carbonyl)-4-methyl-4,5,6,7-tetrahydrothieno[3,2-c]pyridin-2-yl)-1-cyclopropyl-8-(difluoromethoxy)-4-oxo-1,4-dihydro-3-quinolinecarboxylic acid

Ethyl 7-bromo-1-cyclopropyl-8-difluoromethoxy-4-oxo-1,4-dihydro-3-quinolonecarboxylate and Example 16B were processed as described in Example 1 E to provide the desired product as an inseparable mixture of interconverting rotational isomers. MS (APCI(+)) m/z 581 (M+H)⁺; ¹H NMR (300 MHz, DMSO-d₆) 14.63 (s, 0.8H), 14.39 (s, 0.2H), 8.85 (s, 0.8H), 8.80 (s, 0.2H), 8.26 (d, 0.8H), 8.21 (d, 0.2H), 7.98 (d, 1H), 7.71 (s, 1H), 7.71-7.31 (m, 5H), 6.95 (dd, 1H), 5.15 (m, 2H), 4.31 (m, 1H), 4.14 (m, 1H), 3.24 (m, 2H), 2.88 (m, 2H), 1.43 (d, 3H), 1.23-1.03 (m, 4H).

### EXAMPLE 20B

### 1-cyclopropyl-8-(difluoromethoxy)-7-(4-methyl-4,5,6,7-tetrahydrothieno[3,2-c]pyridin-2-yl)-4-oxo-1,4-dihydro-3-quinolinecarboxylic acid hydrobromide

Example 20A was processed as described in Example 11G to provide the desired product. mp 176-180° C. (decomp.); MS (APCI(+)) m/z 447 (M+H)⁺; ¹H NMR (300 MHz, DMSO-d₆) 8.87 (s, 1H), 8.31 (d, 1H), 8.03 (d, 1H), 7.77 (s, 1H), 7.01 (dd, 1H), 4.58 (m, 1H), 4.14 (m, 1H), 3.41 (m, 2H), 3.15 (m, 2H), 1.62 (d, 3H), 1.20-1.03 (m, 4H).

### EXAMPLE 21

### 1-cyclopropyl-7-(7-methyl-4,5,6,7-tetrahydrothieno(2,3-c)pyridin-2-yl)-4-oxo-1,4-dihydro[1,8]naphthyridine-3-carboxylic acid hydrochloride

### EXAMPLE 21A

### N-(2-(3-thienyl)ethyl)acetamide

A solution of 2-(3-thiophene)-ethylamine (6.83 g, 53.7 mmol) in dichloromethane (50 mL) at room temperature was treated with acetyl chloride (4.20 mL, 59.1 mmol), potassium carbonate (44.52 g, 322 mmol) and catalytic tetrabutylammonium iodide, stirred for 5 hours, and partitioned between water and dichloromethane. The organic phase dried (Na₂SO₄), filtered, and concentrated to provide 6.85 g (75%) of the desired product. MS (DCI/NH₃) m/z 170 (M+H)⁺; ¹H NMR (300 MHz, CDCl₃) δ 7.29 (dd, 1H), 7.01 (m, 1H), 6.95 (dd, 1H), 5.62 (m, 1H), 3.51 (q, 2H), 2.85 (t, 2H), 1.94 (s, 3H).

### EXAMPLE 21B

### 7-methyl-4,5,6,7-tetrahydrothieno(2,3-c)pyridine

A solution of Example 21A (1.0 g, 5.9 mmol) in phosphorous oxychloride (6.0 mL, 6.4 mmol) ) at room temperature was treated with phosphorous pentachloride (2.58 g, 12.4 mmol and stirred for 4 hours. The resulting precipitate was collected by filtration and washed with ethyl ether. The solid was suspended in methanol (65 mL), treated with triethylamine (8.42 mL, 6.1 mmol), stirred for 16 hours, cooled to 0° C., treated with sodium borohydride (0.89 g, 23.5 mmol), stirred for 1 hour at 0° C. and at room temperature for 6 hours. The reaction mixture was partitioned between 10% NaOH and dichloromethane, and the organic phase dried (Na₂SO₄) filtered, and concentrated to provide 1.35 g of the crude desired product that was used without further purification. MS (DCI/NH₃) m/z 154 (M+H)⁺; ¹H NMR (300 MHz, CDCl₃) δ 7.10 (d, 1H), 6.77 (d, 1H), 4.14 (m, 1H), 3.32 (m, 1H), 2.99 (m, 1H), 2.65 (m, 2H), 1.45 (d, 3H).

### EXAMPLE 21C

### benzyl 7-methyl-4,7-dihydrothieno(2,3-c)pyridine-6(5H)-carboxylate

Example 21B was processed as described in Example 11C to provide the desired product. MS (DCI/NH₃) m/z 288 (M+H)⁺; ¹H NMR (300 MHz, CDCl₃) δ 7.37 (m, 5H), 7.13 (d, 1H), 6.75 (d, 1H), 5.18 (m, 2H), 4.38 (m, 1H), 3.12 (m, 1H), 2.72 (m, 1H), 2.62 (m, 2H), 1.48 (d, 3H).

### EXAMPLE 21D

### benzyl 7-methyl-2-(tributylstannyl)-4,7-dihydrothieno(2,3-c)pyridine-6(5H)-carboxylate

Example 21C was processed as described in Example 11C to provide the desired product. ¹H NMR (300 MHz, CDCl₃) δ 7.37 (m, 5H), 6.79 (s, 1H), 5.17 (m, 2H), 3.11 (m, 2H), 2.68 (m, 3H), 1.68-0.85 (m, 30H).

### EXAMPLE 21E

### 7-(6-((benzyloxycarbonyl)-7-methyl-4,5,6,7-tetrahydrothieno(2,3-c)pyridin-2-yl)-1-cyclopropyl-4-oxo-1,4-dihydro[1,8]naphthyridine-3-carboxylic acid

Ethyl 1-cyclopropyl-7-chloro-4-oxo-1,4-dihydro[1,8]naphthyridine-3-carboxylate and Example 21D were processed as described in Example 1E to provide the desired product. MS (DCI, NH₃) m/z 544 (M+H)⁺; ¹H NMR (300 MHz, CDCl₃) δ 8.92 (s, 1H), 8.70 (d, 11H), 7.78 (d, 11H), 7.51 (s, 11H), 7.38 (m, 5H), 5.21 (m, 2H), 4.40 (m, 1H), 3.72 (m, 1H), 3.16 (m, 2H), 2.71 (m, 2H), 1.57 (d, 3H), 1.33 (m, 2H), 1.13 (m, 2H).

### EXAMPLE 21F

### 1-cyclopropyl-7-(7-methyl-4,5,6,7-tetrahydrothieno(2,3-c)pyridin-2-yl)-4-oxo-1,4-dihydro[1,8]naphthyridine-3-carboxylic acid hydrochloride

Example 21E was processed as described in Example 17B to provide the desired product. mp 310-312° C.; MS (DCI/NH₃) m/z 382 (m+H)⁺; ¹H NMR (300 MHz, CD₃OD) δ 8.97 (s, 1H), 8.75 (d, 1H), 8.09 (d, 1H), 7.85 (s, 1H), 4.89 (m, 1H), 3.89 (m, 1H), 3.73 (m, 1H), 3.50 (m, 1H), 3.10 (m, 2H), 1.76 (d, 3H), 1.39 (m, 2H), 1.20 (m, 2H).

### EXAMPLE 22

### 1-cyclopropyl-6-fluoro-7-(7-methyl-4,5,6,7-tetrahydrothieno(2,3-c)pyridin-2-yl)-4-oxo-1,4-dihydro[1,8]naphthyridine-3-carboxylic acid hydrochloride

### EXAMPLE 22A

### 7-(6-((benzyloxycarbonyl)-7-methyl-4,5,6,7-tetrahydrothieno(2,3-c)pyridin-2-yl)-1-cyclopropyl-6-fluoro-4-oxo-1,4-dihydro[1,8]naphthyridine-3-carboxylic acid

Ethyl 1-cyclopropyl-7-chloro-6-fluoro-4-oxo-1,4-dihydro[1,8]naphthyridine-3-carboxylate and Example 21D were processed as described in Example 1E to provide the desired product. MS (DCI/NH₃) m/z 559 (M−2)⁺.

### EXAMPLE 22B

### 1-cyclopropyl-6-fluoro-7-(7-methyl-4,5,6,7-tetrahydrothieno(2,3-c)pyridin-2-yl)-4-oxo-1,4-dihydro[1,8]naphthyridine-3-carboxylic acid hydrochloride

Example 22A was processed as described in 17B to provide the desired product. mp 293-294° C.; MS (DCI/NH₃) m/z 400 (M+H)⁺; ¹H NMR (300 MHz, CD₃OD) δ 8.98 (s, 1H), 8.52 (d, 1H), 7.91 (s, 1H), 4.88 (m, 1H), 3.88 (m, 1H), 3.73 (m, 1H), 3.52 (m, 1H), 3.11 (m, 2H), 1.78 (d, 3H), 1.41 (m, 2H), 1.20 (m, 2H).

### EXAMPLE 23

### 1-cyclopropyl-8-methoxy-7-(7-methyl-4,5,6,7-tetrahdrotheino(2,3-c)pyrindin-2-yl)-4-oxo-1,4-dihydro-3-quinolinecarboxylic acid hydrochloride

### EXAMPLE 23A

### 7-(6-((benzyloxycarbonly-7-methyl-4,5,6,7-tetrahydrothieno(2,3-c)pyridin-2-yl)-1-cyclopropyl-8-methoxy-4-oxo-1,4-dihydro-3-quinolinecarboxylic acid

Ethyl 7-bromo-1-cyclopropyl-8-methoxy-4-oxo-1,4-dihydro-3-quinolinecarboxylate and Example 21D were processed as described in Example 1E to provide the desired product. MS (DCI/NH₃) m/z 573 (M+H)⁺; ¹H NMR (300 MHz, CDCl₃) δ 8.91 (s, 1H), 8.23 (d, 1H), 7.74 (d, 1H), 7.38 (m, 5H), 7.33 (s, 1H), 5.21 (m, 2H), 4.68 (m, 1H), 4.10 (m, 1H), 3.67 (s, 3H), 3.17 (m, 2H), 2.70-2.52 (m, 2H), 1.57 (d, 3H), 1.29 (m, 2H), 1.06 (m, 2H).

### EXAMPLE 23B

### 1-cyclopropyl-8-methoxy-7-(7-methyl-4,5,6,7-tetrahydrotheino(2,3-c)pyrindin-2-yl)-4-oxo-1,4-dihydro-3-quinolinecarboxylic acid hydrochloride

Example 23A was processed as described in Example 17B to provide the desired product mp 225-227° C.; MS (DCI/NH₃) m/z 411 (M+H)⁺; ¹H NMR (300 MHz, CD₃OD) δ 8.98 (s, 1H), 8.24 (d, 1H), 7.95 (d, 1H), 7.63 (s, 1H), 4.86 (m, 1H), 4.30 (m, 1H), 3.72 (s, 3H), 3.72 (m, 1H), 3.50 (m, 1H), 3.10 (m, 2H), 1.76 (d, 3H), 1.28 (m, 2H), 1.08 (m, 2H).

### EXAMPLE 24

### 1-cyclopropyl-7-(7,7-dimethyl-4,5,6,7-tetrahydrothieno[3,2-c]pyridin-2-yl)-4-oxo-1,4-dihydro[1,8]naphthyridine-3-carboxylic acid hydrobromide

### EXAMPLE 24A

### 2-methyl-2-(2-thienyl)-1-propanamine

A solution of 2-methyl-2-(2-thienyl)propanenitrile (7.55 g, 49.0 mmol) in THF (50 mL) at 0° C., treated with 1M lithium aluminum hydride in THF (100 mL, 100 mmol), warmed to room temperature over 16 hours, and partitioned between ice water and ethyl acetate. The organic phase was dried (Na₂SO₄), filtered, and concentrated to provide 5.32 g (70%) of the desired product which was used without purification. MS (DCI/NH₃) m/Z 156 (M+H)⁺; ¹H NMR (300 MHz, CDCl₃) 7.17 (dd, 1H), 6.95 (dd, 1H), 6.84 (dd, 1H), 2.77 (s, 2H), 1.36 (s, 6H).

### EXAMPLE 24B

### N-methylene-N-(2-methyl-2-(2-thienyl)propyl)amine

Example 24A was processed as described in Example 1A to provide the desired product. MS (APCI(+)) m/z 168 (M+H)⁺; ¹H NMR (300 MHz, CDCl₃) 7.13 (m, 1H), 6.92 (m, 1H), 6.83 (m, 1H), 3.02 (s, 2H), 2.36 (s, 2H), 1.27 (s, 6H).

### EXAMPLE 24C

### 7,7-dimethyl-4,5,6,7-tetrahydrothieno[3,2-c]pyridine

Example 24B was processed as described in Example 1B to provide the desired product. MS (APCI(+)) m/z 168 (M+H)⁺; ¹H NMR (300 MHz, CDCl₃) 7.07 (d, 1H), 6.69 (d, 1H), 3.58 (s, 2H), 2.65 (m, 2H), 1.35 (s, 6H).

### EXAMPLE 24D

### benzyl 7,7-dimethyl-6,7-dihydrothieno[3,2-c]pyridine-5(4H)-carboxulate

Example 24C was processed as described in Example 11C to provide the desired product. MS (APCI(+)) m/z 302 (M+H)⁺; ¹H NMR (300 MHz, CDCl₃) 7.38-7.30 (m, 5H), 7.12 (d, 1H), 6.75-6.69 (m, 1H), 5.19 (s, 2H), 4.58 (s, 2H), 3.53 (d, 2H), 1.31 (s, 6H).

### EXAMPLE 24E

### benzyl 7,7-dimethyl-2-(tributylstannyl)-6,7-dihydrothieno[3,2-c]pyridine-5(4H)-carboxylate

Example 24D was processed as described in Example 11D to provide the desired product. MS (APCI(+)) m/z 591 (M+H)⁺; ¹H NMR (500 MHz, DMSO-d₆) 7.56-7.33 (m, 5H), 7.26 (s, 1H), 5.18 (s, 2H), 4.61 (s, 2H), 3.53 (m, 2H), 1.63-0.87 (m, 33H).

### EXAMPLE 24F

### 7-(5-((benzyloxy)carbonyl)-7,7-dimethyl-4,5,6,7-tetrahydrothieno[3,2-c]pyridin-2-yl)-1-cyclopropyl-4-oxo- 1.4-dihydro[1,8]naphthyridine-3-carboxylic acid

Ethyl 1-cyclopropyl-7-chloro-4-oxo-1,4-dihydro[1,8]naphthyridine-3-carboxylate and

Example 24E were processed as described in Example 1E to provide the desired product. MS (APCI(+)) m/z 530 (M+H)⁺; ¹H NMR (300 MHz, DMSO-d₆) 8.79 (s, 1H), 8.67 (d, 1H), 8.10 (m, 1H), 7.92 (s, 1H), 7.40-7.30 (m, 5H), 5.16 (s, 2H), 4.57 (m, 2H), 3.85 (m, 1H), 3.55 (s, 2H), 1.32 (s, 6H), 1.30-1.15 (m, 4H).

### EXAMPLE 24G

### 1-cyclopropyl-7-(7,7-dimethyl-4,5,6,7-tetrahydrothieno[3,2-c]pyridin-2-yl)-4-oxo-1,4-dihydro[1,8]naphthyridine-3-carboxylic acid hydrobromide

Example 24F was processed as described in 11G to provide the desired product. mp>300° C. (decomp.); MS (APCI(+)) m/z 396 (M+H)⁺; ¹H NMR (500 MHz, DMSO-d₆, 80° C.) 8.80 (s, 1H), 8.69 (d, 1H), 8.08 (d, 1H), 7.86 (s, 1H), 4.21 (s, 2H), 3.89 (m, 1H), 3.28 (s, 2H), 1.47 (s, 6H), 1.32-1.17 (m, 4H).

### EXAMPLE 25

### 1-cyclopropyl-7-(7,7-dimethyl-4,5,6,7-tetrahydrothieno[3,2-c]pyridin-2-yl)-4-oxo-1,4-dihydro-3-quinolinecarboxylic acid hydrobromide

### EXAMPLE 25A

### 7-(5-((benzyloxy)carbonyl)-7,7-dimethyl-4,5,6,7-tetrahydrothieno[3,2-c]pyridin-2-yl)-1-cyclopropyl-4-oxo-1,4-dihydro-3-quinolinecarboxylic acid

Ethyl 1-cyclopropyl-7-bromo-4-oxo-1,4-dihydroquinolone-3-carboxylate and Example 24E were processed as described in Example 1E to provide the desired product. MS (APCI(−)) m/z 563 (M+Cl)⁻; ¹H NMR (300 MHz, DMSO-d₆) 8.75 (s, 1H), 8.36 (d, 1H), 8.34 (s, 1H), 7.90 (d, 1H), 7.65-7.36 (m, 6H), 5.16 (s, 2H), 4.60 (m, 2H), 3.93 (m, 1H), 3.56 (m, 2H), 1.41-1.15 (m, 10H).

### EXAMPLE 25B

### 1-cyclopropyl-7-(7,7-dimethyl-4,5,6,7-tetrahydrothieno[3,2-c]pyridin-2-yl)-4-oxo-1,4-dihydro-3-quinolinecarboxylic acid hydrobromide

Example 25A was processed as described in 11G to provide the desired product. mp>300° C. (decomp.); MS (APCI(+)) m/z 395 (M+H)⁺; ¹H NMR (300 MHz, DMSO-d₆) 8.77 (s, 1H), 8.40 (d, 1H), 8.35 (d, 1H), 7.92 (dd, 1H), 7.66 (s, 1H), 4.24 (m, 2H), 3.93 (m, 1H), 3.35 (m, 2H), 1.46 (s, 6H), 1.38-1.23 (m, 4H).

### EXAMPLE 26

### 1-cyclopropyl-7-(7,7-dimethyl-4,5,6,7-tetrahydrothieno[3,2-c]pyridin-2-yl)-8-methoxy-4-oxo-1,4-dihydro-3-quinolinecarboxylic acid hydrobromide

### EXAMPLE 26A

### 7-(5-((benzyloxy)carbonyl)-7,7-dimethyl-4,5,6,7-tetrahydrothieno[3,2-c]pyridin-2-yl)-1-cyclopropyl-8-methoxy-4-oxo-1,4-dihydro-3-quinolinecarboxylic acid

Ethyl 1-cyclopropyl-7-bromo-8-methoxy-4-oxo-1,4-dihydroquinolone-3-carboxylate and Example 24E were processed as described in Example 1E to provide the desired product. MS (APCI(−)) m/z 593 (M+Cl)⁻; ¹H NMR (300 MHz, DMSO-d₆) 8.79 (s, 1H), 8.12 (d, 1H), 7.97 (d, 1H), 7.66-7.33 (m, 6H), 5.16 (s, 2H), 4.58 (m, 2H), 4.24 (m, 1H), 3.68 (s, 3H), 3.55 (m, 2H), 1.27 (s, 6H), 1.25-1.00 (m, 4H).

### EXAMPLE 26B

### 1-cyclopropyl-7-(7,7-dimethyl-4,5,6,7-tetrahydrothieno[3,2-c]pyridin-2-yl)-8-methoxy-4-oxo-1,4-dihydro-3-quinolinecarboxylic acid hydrobromide

Example 26A was processed as described in 11G to provide the desired product. mp 212-217° C. (decomp.); MS (APCI(+)) m/z 425 (M+H)⁺; ¹H NMR (300 MHz, DMSO-d₆) 9.10 (br s, 1H), 8.81 (s, 1H), 8.17 (d, 1H), 7.98 (d, 1H), 7.66 (s, 1H), 4.25 (m, 3H), 3.69 (s, 3H), 3.34 (m, 2H), 1.47 (s, 6H), 1.17-1.04 (m, 4H).

### EXAMPLE 27

### 1-cyclopropyl-7-(7,7-dimethyl-4,5,6,7-tetrahydrothieno[3,2-c]pyridin-2-yl)-6-fluoro-4-oxo-1,4-dihydro[1,8]naphthyridine-3-carboxylic acid hydrobromide

### EXAMPLE 27A

### ethyl 7-(5-((benzyloxy)carbonyl)-7,7-dimethyl-4,5,6,7-tetrahydrothieno[3,2-c]pyridin-2-yl)-1-cyclopropyl-6-fluoro-4-oxo- 1,4-dihydro[1,8]naphthyridine-3-carboxylate

Ethyl 1-cyclopropyl-7-chloro-6-fluoro-4-oxo-1,4-dihydro[1,8]naphthyridine-3-carboxylate and Example 24E were processed as described in Example 1E to provide the desired product. MS (APCI(+)) m/z 576 (M+H)⁺; ¹H NMR (300 MHz, DMSO-d₆) 8.56 (s, 1H), 8.30 (d, 1H), 7.79 (d, 1H), 7.66-7.34 (m, 5H), 5.16 (s, 2H), 4.60 (m, 2H), 4.23 (q, 2H), 3.70 (m, 1H), 3.54 (m, 2H), 1.50-1.00 (m, 13H).

### EXAMPLE 27B

### 7-(5-((benzyloxy)carbonyl)-7,7-dimethyl-4,5,6,7-tetrahydrothieno[3,2-c]pyridin-2-yl)-1-cyclopropyl-6-fluoro-4-oxo-1,4-dihydro[1,8]naphthyridine-3-carboxylic acid

Example 27A was processed as described in Example 2B to provide the desired product. MS (APCI(−)) m/z 582 (M+Cl)⁻; ¹H NMR (300 MHz, DMSO-d₆) 8.81 (s, 1H), 8.51 (d, 1H), 7.88 (d, 1H), 7.65-7.30 (m, 5H), 5.16 (s, 2H), 4.60 (m, 2H), 3.85 (m, 1H), 3.57 (m, 2H), 1.50-1.00 (m, 10H).

### EXAMPLE 27C

### 1-cyclopropyl-7-(7,7-dimethyl-4,5,6,7-tetrahydrothieno[3,2-c]pyridin-2-yl)-6-fluoro-4-oxo-1,4-dihydro[1,8]naphthyridine-3-carboxylic acid hydrobromide

Example 27B was processed as described in Example 11 G to provide the desired product. mp 258-262° C. (decomp.); MS (APCI(+)) m/z 414 (M+H)⁺; ¹H NMR (300 MHz, DMSO-d₆) 9.14 (br s, 1H), 8.83 (s, 1H), 8.58 (d, 1H), 7.90 (d, 1H), 4.28 (m, 2H), 3.85 (m, 1H), 3.35 (m, 2H), 1.48 (s, 6H), 1.33-1.18 (m, 4H).

### EXAMPLE 28

### 1-cyclopropl-8-(difluoromethoxy-7-(7,7-dimenhyl-4,5,6,7-tetrqhydrothieno[3,2-c]pyridin-2-yl)-4-oxo-1,4-dihydro-3-quinolinecarboxylic acid hydrobromide

### EXAMPLE 28A

### 7-(5-((benzyloxy)carbonyl)-7,7-dimethyl-4,5,6,7-tetrahydrothieno[3,2-c]pyridin-2-yl)-1-cyclopropyl-8-(difluoromethoxy)-4-oxo-1,4-dihydro-3-quinolinecarboxylic acid

Ethyl 1-cyclopropyl-7-bromo-8-difluoromethoxy-4-oxo-1,4-dihydroquinolone-3-carboxylate and Example 24E were processed as described in Example 1E to provide the desired product. MS (APCI(−)) m/z 629 (M+Cl)¹; ¹H NMR (300 MHz, DMSO-d₆) 8.86 (s, 1H), 8.27 (d, 1H), 7.80 (d, 1H), 7.68-7.33 (m, 6H), 6.95 (dd, 1H), 5.16 (s, 2H), 4.57 (m, 2H), 4.14 (m, 1H), 3.56 (m, 2H), 1.30 (s, 6H), 1.25-1.00 (m, 4H).

### EXAMPLE 28B

### 1-cyclopropyl-8-(difluoromethoxy)-7-(7,7-dieth -4,5,6,7-tetrahydrothieno[3,2-c]pyridin-2-yl)-4-oxo-1,4-dihydro-3-quinolinecarboxylic acid hydrobromide

Example 28A was processed as described in Example 11B to provide the desired product. mp 210-214° C. (decomp.); MS (APCI(+)) m/z 461 (M+H)⁺; ¹H NMR (300 MHz, DMSO-d₆) 9.13 (br s, 1H), 8.87 (s, 1H), 8.31 (d, 1H), 8.08 (d, 1H), 7.60 (s, 1H), 6.99 (t, 1H), 4.25 (m, 2H), 4.15 (m, 1H), 3.36 (m, 2H), 1.46 (s, 6H), 1.28-1.04 (m, 4H).

### EXAMPLE 29

### 1-cyclopropyl-7-(4-methyl-4,5,6,7-tetrhydrothieno(2,3-c)pyridin-2-yl)-4-oxo-1,4-dihydro-3-quinolinecarboxylic acid hydrochloride

### EXAMPLE 29A

### 2-(3-thienyl)propanenitrile

A solution of 3-thiopheneacetonitrile (3.0 g, 24.4 mmol) in THF (15 mL) was added dropwise to a solution comprising 1M lithium bis(trimethylsilyl)amide (25.5 mL, 25.5 mmol) in THF (100 mL) at −70° C., stirred for 30 minutes, warmed to −50° C., stirred for 2 hours, treated dropwise with iodomethane (1.67 mL, 26.8 mmol), warmed to room temperature, and stirred for 18 hours. The reaction mixture was partitioned between 10% NH₄Cl solution and dichloromethane, dried (Na₂SO₄), filtered, and concentrated. The concentrate was purified by flash column chromatography on silica gel with 25% ethyl acetate in hexanes to provide 1.53 g (46%)the desired product. MS (DCI/NH₃) m/z 138 (M+H)⁺; ¹H NMR (300 MHz, CDCl₃) δ 7.36 (dd, 1H), 7.25 (m, 1H), 7.06 (dd, 1H), 3.99 (q, 1H), 1.66 (d, 3H).

### EXAMPLE 29B

### 2-(3-thienyl)propanenitrile

A solution of Example 29A (2.91 g, 21.2 mmol) in THF (10 mL) was added dropwise to a solution of 2M borane-dimethylsulfide in THF (31.8 mL, 63.5 mmol) in THF (100 mL) at 0° C., warmed to room temperature, stirred for 1 hour, and heated at 60° C. for 3 hours, cooled to 0° C., treated dropwise with methanol until evolution of gas ceased, diluted with 6M HCl, heated at 50° C. for 3 hours, cooled to room temperature, poured into water, and adjusted to pH 10 with sodium hydroxide, and extracted with dichloromethane. The extract was dried (Na₂SO₄), filtered, and concentrated to provide 3.32 g of the crude desired product which was used without further purification. MS (DCI/NH₃) m/Z 142 (M+H)⁺; ¹H NMR (300 MHz, CDCl₃) δ 7.28 (m, 1H), 6.99 (m, 2H), 3.63 (m, 1H), 2.85 (m, 4H), 1.27 (d, 3H).

### EXAMPLE 29C

### 4-methyl-4,5,6,7-tetrahydrothieno(2,3-c)pyridine

Example 29B was processed as described in Example 1A and 1B to provide the desired product. MS (DCI/NH₃) m/z 154 (M+H)⁺.

### EXAMPLE 29D

### 4-methyl-6-trityl-4,5,6,7-tetrahydrothieno(2,3-c)pyridine

Example 29C was processed as described in Example 1C to provide the desired product. MS (DCI/NH₃) m/z 396 (M+H)⁺.

### EXAMPLE 29E

### 4-methyl-2-(tributlstannyl)-6-trityl-4,5,6,7-tetrahydrothieno(2,3-c)pyridine

Example 29D was processed as described in Example 1D to provide the desired product.

### EXAMPLE 29F

### 1-cyclopropyl-7-(4-methyl-6-trityl-4,5,6,7-tetrahydrothieno(2,3-c)pyridin-2-yl)-4-oxo-1,4-dihydro-3-quinolinecarboxylic acid

Ethyl 1-cyclopropyl-7-bromo-4-oxo-1,4-dihydroquinoline-3-carboxylate and Example 29E were processed as described in Example 1E to provide the desired product. MS (DCI/NH₃) m/z 623 (M+H)⁺.

### EXAMPLE 29G

### 1-cyclopropyl-7-(4-methyl-4,5,6,7-tetrahydrothieno(2,3-c)pyridin-2-yl)-4-oxo-1,4-dihydro-3-quinolinecarboxylic acid hydrochloride

Example 29F was processed as described in Example 1F to provide the desired product. mp 295-298° C.; MS (DCI/NH₃) m/z 381 (M+H)⁺; ¹H NMR (300 MHz, DMSO-d₆) δ 8.77 (s, 1H), 8.39 (d, 1H), 8.36 (d, 1H), 7.98 (dd, 1H), 7.86 (s, 1H), 4.42 (m, 2H), 3.94 (m, 1H), 3.56 (m, 2H), 3.19 (m, 1H), 1.36 (d, 3H), 1.33 (m, 2H), 1.24 (m, 2H).

### EXAMPLE 30

### 1-cyclopropyl-8-methoxy-7-(4-methyl-4,5,6,7-tetrahydrothieno(2,3-c)pyridin-2-yl)-4-oxo-1,4-dihydro-3-quinolinecarboxylic acid hydrochloride

### EXAMPLE 30A

### 1-cyclopropyl-8-methoxy-7-(4-methyl-6-trilyl-4,5,6,7-tethydrothieno(2,3-c)pyridin-2-yl)-4-oxo-1,4-dihydro-3-quinolinecarboxylic acid

Ethyl 1-cyclopropyl-7-bromo-8-methoxy-4-oxo-1,4-dihydroquinoline-3-carboxylate and Example 29E were processed as described in Example 1E to provide the desired product. MS (DCI/NH₃) m/z 653 (M+H)⁺.

### EXAMPLE 30B

### 1-cyclopropyl-8-methoxy-7-(4-methyl-4,5,6,7-tetrahydrothieno(2,3-c)pyridin-2-yl)-4-oxo-1,4-dihydro-3-quinolinecarboxylic acid hydrochloride

Example 30A was processed as described in Example 1F to provide the desired product. mp 254-259° C.; MS (DCI/NH₃) m/z 411 (M+H)⁺; ¹H NMR (300 MHz, CD₃OD) δ 8.97 (s, 1H), 8.23 (d, 1H), 7.98 (d, 1H), 7.75 (s, 1H), 4.52 (m, 2H), 4.28 (m, 1H), 3.72 (s, 3H), 3.68 (m, 2H), 3.12 (m, 1H), 1.45 (d, 3H), 1.27 (m, 2H), 1.08 (m, 2H).

### EXAMPLE 31

### 1-cyclopropyl-8-(difluoromethoxy)-7-(4-methyl-4,5,6,7-tetrahydrothieno(2,3-c)pyrdin-2-yl)-4-oxo-1,4-dihydro-3-quinolinecarboxylic acid hydrochloride

### EXAMPLE 31A

### 1-cyclopropyl-8-(difluoromethoxy)-7-(4-methyl-6-trityl-4,5,6,7-tetrahydrothieno(2,3-c)pyridin-2-yl)-4-oxo- 1,4-dihydro-3-quinolinecarboxylic acid

Ethyl 1-cyclopropyl-7-bromo-8-difluoromethoxy-4-oxo-1,4-dihydroquinoline-3-carboxylate and Example 29E were processed as described in Example 1E to provide the desired product. MS (DCI/NH₃) m/z 689 (M+H)⁺; ¹H NMR (300 MHz, CDCl₃) δ 8.93 (s, 1H), 8.37 (d, 1H), 7.72 (d, 1H), 7.56 (m, 5H), 7.45 (s, 1H), 7.31 (m, 5H), 7.20 (m, 5H), 6.66 (dd, 1H), 4.25 (m, 2H), 3.95 (m, 1H), 3.25 (m, 1H), 3.16 (m, 2H), 1.34 (d, 3H), 1.29 (m, 2H), 1.04 (m, 2H).

### EXAMPLE 31B

### 1-cyclopropyl-8-(difluoromethoxy)-7-(4-methyl-4,5,6,7-tetrahydrothieno(2,3-c)pyridin-2-yl)-4-oxo-1,4-dihydro-3-quinolinecarboxylic acid hydrochloride

Example 31A was processed as described in Example 1F to provide the desired product. mp 218-222° C.; MS (DCI/NH₃) m/z 447 (M+H)⁺; ¹H NMR (300 MHz, CD₃OD) δ 9.00 (s, 1H), 8.41 (d, 1H), 7.92 (d, 1H), 7.68 (s, 1H), 6.62 (dd, 1H), 4.52 (m, 2H), 4.27 (m, 1H), 3.65 (m, 2H), 3.14 (m, 1H), 1.45 (d, 3H), 1.31 (m, 2H), 1.09 (m, 2H).

### EXAMPLE 32

### 1-cyclopropyl-7-(4-methyl-4,5,6,7-tetrahydrothieno(2,3-c)pyrdin-2-yl)-4-oxo-1,4-dihydro[1,8]naphthyridine-3-carboxylic acid hydrochloride

### EXAMPLE 32A

### 1-cyclopropyl-7-(4-methyl-6-trityl-4,5,6,7-tetrahydrothieno(2,3-c)pyridin-2-yl)-4-oxo-1,4-dihydro[1,8]naphthyridine-3-carboxylic acid

Ethyl 1-cyclopropyl-7-chloro-4-oxo-1,4-dihydro[1,8]naphthyridine-3-carboxylate and Example 29E were processed as described in Example 1E to provide the desired product. MS (DCI/NH₃) m/z 625 (M+H)⁺.

### EXAMPLE 32B

### 1-cyclopropyl-7-(4-methyl-4,5,6,7-tetrahydrothieno(2,3-c)pyridin-2-yl)-4-oxo-1,4-dihydro[1,8]naphthyridine-3-carboxylic acid hydrochloride

Example 32A was processed as described in Example 1F to provide the desired product. mp 284-286° C.; MS (DCI/NH₃) m/z 382 (M+H)⁺; ¹H NMR (300 MHz, CD₃OD) δ 8.97 (s, 1H), 8.75 (d, 1H), 8.13 (d, 1H), 8.00 (s, 1H), 4.53 (m, 2H), 3.88 (m, 1H), 3.69 (m, 2H), 3.13 (m, 1H), 1.46 (d, 3H), 1.38 (m, 2H), 1.20 (m, 2H).

### EXAMPLE 33

### 1-cyclopropyl-6-fluoro-7-(4-methyl-4,5,6,7-tetrahydrothieno(2,3-c)pyridin-2-yl)-4-oxo-1,4-dihydro[1,8]naphthyridine-3-carboxylic acid hydrochloride

### EXAMPLE 33A

### 1-cyclopropyl-6-fluoro-7-(4-methyl-6-trityl-4,5,6,7-tetrahydrothieno(2,3-c)pyridin-2-yl)-4-oxo-1,4-dihydro[1,8]naphthyridine-3-carboxylic acid

### 1-cyclopropyl-6-fluoro-7-(4-methyl-6-trityl-4,5,6,7-tetrahydrotheino(2,3-c)pyrindin-2-yl)-4-oxo-1,4-dihydro-3-naphtheridinecarboxylic acid

Ethyl 1-cyclopropyl-7-chloro-6-fluoro-4-oxo-1,4-dihydro[1,8]naphthyridine-3-carboxylate and Example 29E were processed as described in Example 1E to provide the desired product. MS (DCI/NH₃) m/z 642 (M+H)⁺; ¹H NMR (300 MHz, CDCl₃) δ 8.90 (s, 1H), 8.38 (d, 1H), 7.86 (d, 1H), 7.57 (m, 5H), 7.30 (m, 5H), 7.19 (m, 5H), 3.97 (m, 2H), 3.76 (m, 1H), 3.62 (m, 1H), 3.17 (m, 2H), 1.27 (d, 3H), 1.14 (m, 2H), 1.05 (m, 2H).

### EXAMPLE 33B

### 1-cyclopropyl-6-fluoro-7-(4-methyl-4,5,6,7-tetrahydrothieno(2,3-c)pyridin-2-yl)-4-oxo-1,4-dihydror[1,8]naphthyridine-3-carboxylic acid hydrochloride

Example 33A was processed as described in Example 1F to provide the desired product. mp 288-290° C.; MS (DCI/NH₃) m/z 400 (M+H)⁺; ¹H NMR (300 MHz, CD₃OD) δ 8.98 (s, 1H), 8.52 (d, 1H), 8.01 (s, 1H), 4.57 (m, 2H), 3.87 (m, 1H), 3.69 (m, 2H), 3.14 (m, 1H), 1.45 (d, 3H), 1.40 (m, 2H), 1.22 (m, 2H).

### EXAMPLE 34

### 1-cyclopropyl-7-(4,4-dimethyl-4,5,6,7-tetrahydrothieno(2,3-c)pyridin-2-yl)-8-methoxy-4-oxo-1,4-dihydro-3 -quinolinecarboxylic acid hydrochloride

### EXAMPLE 34A

### 2-methyl-2-(3 -thienyl)propanenitrile

A solution of 3-thiopheneacetonitrile (3.0 g, 24.4 mmol) in THF (50 mL) was treated with 1M lithium bis(trimethylsilyl)amide in THF (51.2 mL, 51.2 mmol) at −50° C., stirred for 2 hours, treated with iodomethane (3.18 mL, 51.1 mmol), warmed to room temperature, stirred for 2 hours, and partitioned between 10% ammonium chloride and dichloromethane. The organic fraction was dried (Na₂SO₄), filtered, and concentrated. The concentrate was purified by flash column chromatography on silica gel with 15% ethyl acetate in hexanes to provide 2.40 g (65%) of the desired product. MS (DCI/NH₃) M/Z 152 (M+H)⁺; ¹H NMR (300 MHz, CDCl₃) δ 7.36 (dd, 1H), 7.25 (m, 1H), 7.12 (dd, 1H), 1.72 (s, 6H).

### EXAMPLE 34B

### 2-methyl-2-(3-thienyl)-1-propanamine

Example 34A was processed as described in Example 29B to provide the desired product. MS (DCI/NH₃) m/z 156 (M+H)⁺.

### EXAMPLE 34C

### 4,4-dimethyl-2-(tributylstannyl)-6-trityl-4,5,6,7-tetrahydrothieno(2,3-c)pyridine

Example 34B was processed as described in Examples 1A, 1B, 1C, and 1D to provide the desired product.

### EXAMPLE 34D

### 1-cyclopropyl-7-(4,4-dimethyl-6-trityl-4,5,6,7-tetrahydrothieno(2,3-c)pyridin-2-yl)-8-methoxy-4-oxo-1,4-dihydro-3-quinolinecarboxylic acid

Ethyl 1-cyclopropyl-7-bromo-8-methoxy-4-oxo-1,4-dihydroquinoline-3-carboxylate and Example 34C were processed as described in Example 1E to provide the desired product. MS (DCI/NH₃) m/z 667 (M+H)⁺.

### EXAMPLE 34E

### 1-cyclopropyl-7-(4,4-dimethyl-4,5,6,7-tetrahydrothieno(2,3-c)pyridin-2-yl)-8-methoxy-4-oxo-1,4-dihydro-3-quinolinecarboxylic acid hydrochloride

Example 34D was processed as described in Example 1F to provide the desired product. MS (DCI/NH₃) m/z 425 (M+H)⁺; ¹H NMR (300 MHz, CDCl₃) δ 8.94 (s, 1H), 8.28 (d, 1H), 7.78 (d, 1H), 7.49 (s, 1H), 4.54 (m, 2H), 4.10 (m, 1H), 3.30, (m, 2H), 1.56 (s, 6H), 1.30 (m, 2H), 1.05 (m, 2H).

### EXAMPLE 35

### 1-cyclopropyl-6-fluoro-7-(4-hydroxy-4,5,6,7-tetrahydro-1-benzothien-2-yl)-4-oxo-1,4-dihydro[1,8]naphthyridine-3-carboxylic acid

### EXAMPLE 35A

### ((2-bromo-4,5,6,7-tetrahydro-1-benzothien-4-yl)oxy)(tert-butyl)dimethylsilane

A solution of 2-bromo-4-keto-4,5,6,7-tetrahydrothianapthene (prepared by the method of Pinna, et. al. Eur. J Med. Chem. Chim. Ther 1994, 447-54) (0.50 g, 2.16 mmol) in methanol (20 mL) at 0° C., treated dropwise with a solution of sodium borohydride (0.082 g, 2.16 mmol) in 2M NaOH (3 mL), warmed to room temperature, stirred for 1 hour, treated dropwise with 5% HCl until evolution of hydrogen ceased, and partitioned between ethyl acetate and brine. The aqueous layer was extracted with ethyl acetate, and the combined extracts were washed sequentially with water, 5% HCl, and brine, dried (MgSO₄), filtered, and concentrated to provide 0.495 g (98%) of the desired product as a yellow oil.

A solution of the yellow oil in DMF (10 mL) was treated with imidazole (0.215 g, 3.15 mmol), tert-butyldimethylchlorosilane (0.412 g, 2.73 mmol), and several crystals of DMAP, stirred for 12 hours at room temperature, and poured into saturated ammonium chloride. The layers were separated, and the aqueous layer was extracted with ethyl acetate. The combined extracts were washed with water and brine, dried (MgSO₄), filtered, and concentrated. The concentrate was purified by flash column chromatography on silica gel with 5% then 10% ethyl acetate in hexanes to provide 0.676 g (91%) of the desired product as an amber oil. MS (DCI/NH₃) m/z 364 (M+NH₄)⁺; ¹H NMR ( 300 MHz, CDCl₃) 6.84 (s, 1H), 4.68 (m, 1H), 2.65-2.53 (m, 2H), 2.10-1.87 (m, 2H), 1.84-1.65 (m, 2H), 0.93 (s, 9H), 0.15 (s, 3H), 0.13 (s, 3H).

### EXAMPLE 35B

### 4-(1-methyl-1-(trimethylsilyl)ethoxy)-4,5,6,7-tetrahydro-1-benzothien-2-ylboronic acid

A solution of Example 35A (1.032 g, 2.97 mmol) in THF (60 mL) at −78 ° C. was treated dropwise with 1.6M n-butyllithium in hexanes (1.4 mL, 3.56 mmol), stirred for 1.5 hours, treated dropwise with triisopropylborate (1.0 mL, 4.45 mmol), stirred for 30 minutes, warmed to room temperature, and stirred for 2 hours. The reaction mixture at 0° C., treated dropwise with 5% HCl until pH 2, and partitioned between ethyl acetate and water. The aqueous phase was extracted with ethyl acetate, and the combined extracts washed with brine, dried (MgSO₄), filtered, and concentrated. The concentrate was purified by flash column chromatography on silica gel with 20% then 33% ethyl acetate in hexanes to provide 0.355 g (38%) of the desired product as a slightly yellow solid. MS (APCI(−)) m/z 347 (M+Cl)⁻; ¹H NMR (300 MHz, DMSO-d₆) 8.04 (s, 2H), 7.46 (s, 1H), 4.77 (m, 1H), 2.83-2.58 (m, 2H), 1.97-1.57 (m, 4H), 0.89 (s, 9H), 0.14 (s, 3H).

### EXAMPLE 35C

### ethyl 7-(4-((tert-butyl(dimethyl)silyl)oxy)-4,5,6,7-tetrahydro-1-benzothien-2-yl)-1-cyclopropyl-6-fluoro-4-oxo- 1,4-dihydro[1,8naphthnridine-3-carboxylate

Example 35B (0.102 g, 0.326 mmol), ethyl 1-cyclopropyl-7-chloro-6-fluoro-4-oxo-1,4-dihydro[1,8]naphthyridine-3-carboxylate (0.078 g, 0.251 mmol), cesium carbonate (0.123 g, 0.377 mmol), and Pd(PPH₃)₂Cl₂ (0.018 g, 0.025 mmol) were combined in DMF (5 m), heated to 100° C. for 12 hours, cooled to room temperature, diluted with ethyl acetate and saturated ammonium chloride, and filtered through diatomaceous earth (Celite®). The layers were eparated, and the aqueous layer was extracted with ethyl acetate. The combined extracts were washed with brine, dried (MgSO₄), filtered, and concentrated. The concentrate was purified by flash column chromatography on silica gel with 50% ethyl acetate in hexanes to provide 0.042 g (31%) of the desired product as a tan solid. MS (APCI(+)) m/z 543 (M+H)⁺; ¹H NMR (300 MHz, DMSO-d₆) 8.56 (s, 1H), 8.30 (d, 1H), 7.80 (d, 1H), 4.86 (m, 1H), 4.24 (q, 2H), 3.66 (m, 1H), 2.94-2.70 (m, 2H), 2.03-1.61 (m, 2H), 1.29 (t, 3H), 1.22 (m, 2H), 1.09 (m, 2H), 0.91 (s, 9H), 0.18 (s, 3H), 0.15 (s, 3H).

### EXAMPLE 35D

### 7-(4-((tert-butyl(dimethyl)silyl)oxy)-4,5,6,7-tetrahydro-1-benzothien-2-yl)-1-cyclopropyl-6-fluoro-4-oxo-1,4-dihydro[1,8]naphthyridine-3-carboxylic acid

Example 35C was processed according to Example 2B to provide the desired product. MS (APCI(+)) m/z 515 (M+H)⁺; MS (APCI(−)) m/z 549 (M+Cl)⁻; ¹H NMR (300 MHz, DMSO-d₆) 14.53 (s, 1H), 8.80 (s, 1H), 8.53 (d, 1H), 7.87 (d, 1H), 4.87 (m, 1H), 3.82 (m, 1H), 2.83 (m, 2H), 2.05-1.61 (m, 4H), 1.31-1.13 (m, 4H), 0.91 (s, 9H), 0.19 (s, 3H), 0.16 (s, 3H).

### EXAMPLE 35E

### 1-cyclopropyl-6-fluoro-7-(4-hydroxy-4,5,6,7-tetrahydro-1-benzothien-2-yl)-4-oxo-1,4-dihydro[1,8]naiphthyridine-3-carboxylic acid

A solution of Example 35D (0.038 g, 0.074 mmol) in THF (5 mL) at 0° C. was treated with 1M tetrabutylammonium fluoride in THF (0.30 mL, 0.30 mmol), warmed to room temperature, and stirred for 3 hours. The reaction mixture was diluted with saturated ammonium chloride and extracted with ethyl acetate. The extracts were washed with brine, dried (Na₂SO₄), filtered, and concentrated. The concentrate was triturated with 5% ethyl ether in pentane, filtered, and washed with pentane to provide 0.020 g (67%) of the desired product as a yellow solid. MS (APCI(+)) m/z 401 (M+H)⁺; MS (APCI(−)) m/z 435 (M+Cl)⁻; ¹H NMR (300 MHz, DMSO-d₆) 14.55 (br s, 1H), 8.80 (s, 1H), 8.51 (d, 1H), 7.97 (d, 1H), 5.29 (m, 1H), 4.63 (m, 1H), 3.83 (m, 1H), 2.88-2.70 (m, 2H), 2.03-1.84 (m, 2H), 1.84-1.59 (m, 2H), 1.35-1.23 (m, 2H), 1.21-1.12 (m, 2H).

### EXAMPLE 36

### 1-cyclopropyl-7-(4-hydroxy-5,6-dihydro-4H-thieno[2,3-b]thiopyran-2-yl)-8-methoxy-4-oxo-1,4-dihydro-3-quinolinecarboxylic acid

### EXAMPLE 36A

### 2-bromo-5,6-dihydro-4H-thieno[2,3-b]thiopyran-4-one

A solution of 5,6-dihydro-4H-thieno[2,3-b]thiopyran-4-one (0.74 g, 4.35 mmol) (prepared by the method of Jones, et. al., J. Org. Chem. 1991, 56, 763) in 50% aqueous acetic acid (10 ml) at 0° C. was treated dropwise with a solution of bromine (0.76 g, 4.78 mmol) in acetic acid (5 ml), stirred for 30 minutes, and treated dropwise with saturated aqueous sodium acetate solution until precipitation was complete. The concentrate was collected by filtration, washed with water, and dried to provide 0.56 g (52%) of the desired product as a yellow-green solid. MS (APCI(+)) m/z 249 (M+H)⁺; 1H NMR (300 MHz, CDCl₃) δ 7.41 (s, 1H), 3.37 (m, 2H), 2.84 (m, 2H).

### EXAMPLE 36B

### ((2-bromo-5,6-dihydro-4H-thieno[2,3-b]thiopyran-4-yl)oxy)(tert-butyl)dimethylsilane

Example 36A was processed as described in Example 35A to provide the desired product. ¹H NMR (300 MHz, CDCl₃) δ 6.80 (s, 1H), 4.71 (dd, 1H), 3.25 (ddd, 1H), 2.91 (ddd, 1H), 2.18 (m, 1H), 2.08 (m, 1H), 0.90 (s, 3H), 0.13 (s, 3H), 0.12 (s, 3H).

### EXAMPLE 36C

### tert-butyl(dimethyl)((2-(tributylstannyl)-5,6-dihydro-4H-thieno[2,3-b]thiopyran-4-yl)oxy)silane

A solution of Example 36B (0.71 g, 1.94 mmol) in THF (20 ml) at -78 ° C. was treated dropwise with 1.6M n-butyllithium in hexanes (1.3 ml, 2.14 mmol), stirred for 1 hour, warmed to −30° C. for 30 minutes, recooled to -78° C, and treated dropwise with chlorotributylstannane (0.70 g, 2.14 mmol). The reaction mixture was warmed to room temperature over 12 hours, partitioned between saturated ammonium chloride and ethyl acetate, and extracted with ethyl acetate. The combined extracts were washed with water and brine, dried (MgSO₄), filtered, and concentrated to provide 1.12 g (100%) of the desired product as an amber oil which was used without further purification. ¹H NMR (300 MHz, CDCl₃) δ 6.97 (s, 1H), 4.89 (dd, 1H), 3.27 (ddd, 1H), 2.92 (ddd, 1H), 2.22 (m, 1H), 2.05 (m, 1H), 1.71-1.46 (m, 6H), 1.40-1.24 (m, 6H), 1.13-1.01 (m, 6H), 0.90 (s, 9H), 0.89 (t, 9H), 0.14 (s, 3H), 0.10 (s, 3H).

### EXAMPLE 36D

### ethyl 7-(4-((tert-butyl(dimethyl)sily)oxy)-5,6-dihydro-4H-thieno[2,3-b]thiopyran-2-yl)-1-cyclopropyl-8-methoxy-4-oxo-1,4-dihydro-3-quinolinecarboxylate

Ethyl 1-cyclopropyl-7-bromo-8-methoxy-4-oxo-1,4-dihydroquinoline-3-carboxylate and Example 36C were processed as described in Example 1E to provide the desired product. ¹H NMR (300 MHz, CDCl₃) δ 8.62 (s, 1H), 8.22 (d, 1H), 7.56 (d, 1H), 7.43 (s, 1H), 4.87 (dd, 1H), 4.40 (q, 2H), 3.95 (m, 1H), 3.64 (s, 3H), 3.34 (ddd, 1H), 3.00 (ddd, 1H), 2.26 (m, 1H), 2.12 (m, 1H), 1.41 (t, 3H), 1.18 (m, 2H), 0.97 (m, 2H), 0.93 (s, 9H), 0.18 (s, 3H), 0.17 (s, 3H).

### EXAMPLE 36E

### 7-(4-((tert-butyl(dimethyl)silyl)oxy)-5,6-dihydro-4H-thieno[2,3-b]thiopyran-2-yl)-1-cyclopropyl-8-methoxy-4-oxo-1,4-dihydro-3-quinolinecarboxylic acid

Example 36D was processed as described in Example 2B to provide the desired product. MS (APCI(+)) m/z 544 (M+H)⁺; ¹HNMR (300 MHz, DMSO-d₆) δ 8.78 (s, 1H), 8.12 (d, 1H), 7.93 (d, 1H), 7.69 (s, 1H), 4.96 (m, 1H), 4.24 (m, 1H), 3.68 (s, 3H), 3.35-3.11 (m, 2H), 2.23-2.09 (m, 2H), 2.08-1.95 (m, 2H), 1.14 (m, 2H), 1.02 (m, 2H), 0.89 (s, 9H), 0.19 (s, 3H), 0.17 (s, 3H).

### EXAMPLE 36F

### 1-cyclopropyl-7-(4-hydroxy-5,6-dihydro-4H-thieno[2,3-b]thiopyran-2-yl)-8-methoxy-4-oxo-1,4-dihydro-3-quinolinecarboxylic acid

Example 36E was processed as described in Example 35E to provide the desired product. MS (APCI(+)) m/z 444 (M+H)⁺; ¹H NMR (300 MHz, DMSO-d₆) 14.94 (s, 1H), 8.78 (s, 1H), 8.11 (d, 1H), 8.00 (d, 1H), 7.81 (s, 1H), 5.38 (d, 1H). 4.72 (q, 1H), 4.24 (m, 1H), 3.69 (s, 3H), 3.30-3.08 (m, 2H), 2.24-2.09 (m, 1H), 2.07-1.93 (m, 1H), 1.16 (m, 2H), 1.03 (m, 2H).

### EXAMPLE 37

### 7-(4-amino-5,6-dihydro-4H-thieno[2,3-b]thiopyran-2-yl)-1-cyclopropyl-8-methoxy-4-oxo-1,4-dihydro-3-quinolinecarboxylic acid hydrochloride

### EXAMPLE 37A

### ethyl 1-cyclopropyl-7-(4-hydroxy-5,6-dihydro-4H-thieno[2,3-b]thiopyran-2-yl)-8-methoxy-4-oxo- 1,4-dihydro-3-quinolinecarboxylate

Example 36D was processed as described in Example 35E to provide the desired product. MS (APCI(+)) m/z 458 (M+H)⁺; ¹H NMR (300 MHz, CDCl₃) 8.63 (s, 1H), 8.18 (d, 1H), 7.58 (d, 1H), 7.55 (s, 1H), 4.90 (m, 1H), 4.40 (q, 2H), 3.94 (m, 1H), 3.64 (s, 3H), 3.36 (dt, 1H), 2.99 (ddd, 1H), 2.47-2.37 (m, 1H), 2.33 (br s, 1H), 2.12 (m, 1H), 1.41 (t, 3H), 1.19 (m, 2H), 0.98 (m, 2H).

### EXAMPLE 37B

### ethyl 7-(4-azido-5,6-dihydro-4H-thieno[2,3-b]thiopyran-2-yl)-1-cyclopropyl-8-methoxy-4-oxo-1,4-dihydro-3-quinolinecarboxylate

A solution of Example 37A (0.110 g, 0.240 mmol) in 1:1 toluene:dichloromethane (7 ml) was treated with sequentially with DPPA (0.104 ml, 0.481 mmol) and DBU (83 mL, 0.553 mmol), stirred for 16 hours at room temperature, and partitioned between ethyl acetate and saturated ammonium chloride. The aqueous layer was extracted with ethyl acetate, and the combined extracts were washed with brine, dried (Na₂SO₄), filtered, and concentrated. The residue was purified by flash column chromatography on silica gel with 3% methanol in dichloromethane to provide 0.096 g (83%) of the desired product as an off-white solid. MS (APCI(+)) m/z 483 (M+H)⁺; ¹H NMR (300 MHz, CDCl₃) 8.63 (s, 1H), 8.23 (d, 1H), 7.60 (d, 1H), 7.49 (s, 1H), 4.72 (t, 1H), 4.40 (q, 2H), 3.96 (m, 1H), 3.65 (s, 3H), 3.40-3.22 (m, 1H), 3.03 (ddd, 1H), 2.48-2.35 (m, 1H), 2.26-2.11 (m, 1H), 1.41 (s, 3H), 1.19 (m, 2H), 0.97 (m, 2H).

### EXAMPLE 37C

### 7-(4-amino-5,6-dihydro-4H-thieno[2,3-b]thiopyran-2-yl)-1-cyclopropyl-8-methoxy4-oxo-1,4-dihydro-3-quinolinecarboxylic acid hydrochloride

A solution of Example 37B (0.095 g, 0.197 mmol) and triphenylphosphine (0.155 g, 0.591 mmol) in 10:1 THF:water (10 ml) was heated to reflux for 4 hours, cooled to room temperature, treated with methanol (0.5 mL) and di-(tert-butyl)dicarbonate (0.065 g, 0.296 mmol), and stirred for 12 hours. The reaction mixture was partitioned between ethyl acetate and saturated ammonium chloride, and the organic phase was washed with water and brine, dried (Na₂SO₄), filtered, and concentrated. The residue was purified by flash column chromatography on silica gel with 33% acetone in hexanes to provide the desired product as an inseparable mixture with triphenylphosphine oxide.

The mixture was processed as described in Example 2B to provide a carboxylic acid that was used without further purification.

A solution of the carboxylic acid in dichloromethane (3 ml) was treated with 4M HCl in dioxane (3 ml), heated to reflux, cooled to room temperature, and treated with hexanes (1 mL). The resulting precipitate was filtered, washed with hexanes, and dried to provide 0.035 g (38% over three steps) of the desired product as a tan solid. MS (APCI(−)) m/z 463 (M+Cl)⁻; ¹H NMR (300 MHz, DMSO-d₆) 8.80 (s, 1H), 8.60 (br s, 3H), 8.18 (d, 1H), 8.10 (s, 1H), 7.89 (d, 1H), 4.60 (m, 1H), 4.25 (m, 1H), 3.71 (s, 3H), 3.42-3.20 (m, 2H), 2.43-2.24 (m, 2H), 1.17 (m, 2H), 1.04 (m, 2H).

### EXAMPLE 38

### 1-cyclopropyl-7-(4-hydroxy-4,5,6,7-tetrahydro-1-benzothien-2-yl)-8-methoxy-4-oxo-1,4-dihydro-3-quinolinecarboxylic acid

### EXAMPLE 38A

### tert-butyl(dimethyl)(4,5,6,7-tetrahydro-1-benzothien-4-yloxy)silane

4-Keto-4,5,6,7-tetrahydrothianapthene was processed as described in Example 35A to provide the desired product. MS (DCI/NH₃) m/z 269 (M+H)⁺; ¹H NMR (300 MHz, CDCl₃) δ 7.04 (d, 1H), 6.93 (d, 1H), 4.78 (m, 1H), 2.85-2.65 (m, 2H), 2.11-1.92 (m, 2H), 1.85-1.69 (m, 2H), 0.92 (s, 9H), 0.14 (s, 3H), 0.12 (s, 3H).

### EXAMPLE 38B

### tert-butyl(dimethy)((2-(tributylstannyl)-4,5,6,7-tetrahydro-1-benzothien-4-yl)oxysilane

Example 38A was processed as described in Example 1D to provide the desired product. ¹H NMR (300 MHz, CDCl₃) δ 7.02 (s, 1H), 4.82 (m, 1H), 2.78 (m, 2H), 2.00 (m, 2H), 1.73 (m, 2H), 1.56 (m, 6H), 1.33 (m, 6H), 1.05 (m, 6H), 0.93 (s, 9H), 0.89 (t, 9H), 0.12 (s, 3H), 0.15 (2, 3H).

### EXAMPLE 38C

### 7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylic acid

Ethyl 7-bromo-1-cyclopropyl-8-methoxy-4-oxo-1,4-dihydro-3 -quinolonecarboxylate and Example 38B were processed as described in Example 1E to provide the desired product. MS (DCI/NH₃) m/z 526 ((M+H)⁺; ¹H NMR (300 MHz, CDCl₃) δ 14.78 (s, 1H), 8.90 (s, 1H), 8.23 (d, 1H), 7.71 (d, 1H), 7.55 (s, H1H), 4.83 (m, 1H), 4.11 (m, 1H), 3.67 (s, 3H), 2.93-2.72 (m, 2H), 2.13-1.99 (m, 2H), 1.88-1.72 (m, 2H), 1.32 (m, 2H), 1.06 (m, 2H), 0.96 (s, 9H), 0.22 (s, 3H), 0.18 (s, 3H).

### EXAMPLE 38D

### 1-cyclopropyl-7-(4-hydroxy-4,5,6,7-tetrahydro-1-benzothien-2-yl)-8-methoxy-4-oxo-1,4-dihydro-3-quinolinecarboxvlic acid

Example 38C was processed as described in Example 35E to provide the desired product. mp 237-239° C.; MS (DCI/NH₃) m/z 412 (M+H)⁺; ¹H NMR (300 MHz, CDCl₃) δ 8.91 (s, 1H), 8.23 (d, 1H), 7.77 (d, 1H), 7.63 (s, 1H), 4.85 (m, 1H), 4.10 (m, 1H), 3.68 (s, 3H), 3.38 (m, 1H), 2.97-2.74 (m, 2H), 2.11-2.02 (m, 2H), 1.96-1.83 (m, 2H), 1.28 (m, 2H), 1.04 (m, 2H).

### EXAMPLE 39

### 7-(4-azido-4,5,6,7-tetrahydro-1-benzothien-2-yl)-1-caclopropyl-8-methoxy-4-oxo-1,4-dihydro-3-quinolinecarboxylic acid

### EXAMPLE 39A

### methyl 7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylate

A solution of Example 38C (2.15 g, 4.10 mmol) in 1:1 methanol:THF (50 mL) at 0° C. was treated with 2M trimethylsilyldiazomethane in hexanes (9.70 mL, 19.4 mmol), warmed to room temperature over 5 hours, treated with acetic acid (15 drops), and poured into water. The layers were separated, and the aqueous layer was extracted with dichloromethane. The combined extracts were dried (Na₂SO₄), filtered, and concentrated. The concentrate was purified by flash column chromatography on silica gel with 1% methanol in dichloromethane to provide 1.68 g (75%) of the desired product as a tan solid. MS (DCI/NH₃) m/z 540 (M+H)⁺; ¹H NMR (300Hz, CDCl₃) δ 8.65 (s,1H), 8.22 (d, 11H), 7.58 (d, 11H), 7.48 (s, 1H), 4.83 (m, 1H), 3.98 (m, 1H), 3.93 (s, 3H), 3.13 (s, 3H), 2.80 (m, 2H), 2.04 (m, 2H), 1.80 (m, 2H), 1.18 (m, 2H), 0.98 (m, 2H), 0.95 (s, 9H), 0.21 (s, 3H), 0.18 (s, 3H).

### EXAMPLE 39B

### methyl 1-cyclopropyl-7-(4-hydroxy-4,5,6,7-tetrahydro-1-benzothien-2-yl)-8-methoxy-4-oxo-1.4-dihydro-3-quinolinecarboxylate

Example 39A was processed as described in Example 35E to provide the desired product. MS (DCI/NH₃) m/z 426 (M+H)⁺; ¹H NMR (300 MHz, CDCl₃) δ 8.65 (s, 1H), 8.22 (d, 1H), 7.63 (d, 1H), 7.56 (s, 1H), 4.84 (m, 1H), 3.98 (m, 1H), 3.92 (s, 3H), 3.64 (s, 3H), 3.25 (m, 1H), 2.96-2.72 (m, 2H), 2.07 (m, 2H), 1.89 (m, 2H), 1.20 (m, 2H), 0.98 (m, 2H).

### EXAMPLE 39C

### methyl 7-(4-azido-4,5,6,7-tetrahydro-1-benzothien-2-yl)-1-cyclopropyl-8-methoxy-4-oxo-1,4-dihydro-3-quinolinecarboxylate

Example 39B was processed as described in Example 37B to provide the desired product. MS (DCI/NH₃) m/z 451 (M+H)⁺; ¹H NMR (300 MHz, CDCl₃) δ 8.66 (s, 1H), 8.22 (d, 1H), 7.62 (d, 1H), 7.46 (s, 1H), 5.95 (m, 1H), 3.97 (m, 1H), 3.93 (s, 3H), 3.63 (s, 3H), 3.01-2.72 (m, 2H), 2.05-1.92 (m, 4H), 1.20 (m, 2H), 0.97 (m, 2H).

### EXAMPLE 39D

### 7-(4-azido-4,5,6,7-tetrahydro-1-benzothien-2-yl)-1-cyclopropyl-8-methoxy-4-oxo-1,4-dihydro-3-quinolinecarboxylic acid

Example 39C was processed as described in Example 2B to provide the desired product. mp 134-136° C.; MS (DCI/NH₃) m/z 437 (M+H)⁺; ¹H NMR (300 MHz, CD₃OD) δ 8.96 (s, 1H), 8.22 (d, 1H), 7.90 (d, 1H), 7.67 (s, 1H), 4.67 (m, 1H), 4.31 (m, 1H), 3.62 (s, 3H), 3.02-2.77 (m, 2H), 2.23-1.94 (m, 4H), 1.29 (m, 2H), 1.10 (m, 2H).

### EXAMPLE 40

### 7-(4-amino-4,5,6,7-tetrahydro-1-benzothien-2-yl)-1-cyclopropyl-8-methoxy-4-oxo-1,4-dihydro-3-quinolinecarboxylic acid hydrochloride

### EXAMPLE 40A

### methyl 7-(4-((tert-butoxycarbonyl)amino)-4,5,6,7-tetrahydro-1-benzothien-2-yl-1-cyclopropyl-8-methoxy-4-oxo-1,4-dihydro-3-quinolinecarboxylate

A solution of Example 39C (0.30 g, 0.7 mmol) in 10:1 THF:water (33 mL) was treated with triphenylphosphine (0.70 g, 2.7 mmol), heated at 60° C. for 17 hours, cooled, treated sequentially with sodium bicarbonate (0.22 g, 2.6 mmol) and di-(tert-butyl)dicarbonate (0.26 g, 1.2 mmol), stirred for 6 hours, and poured into water. The layers were separated, and the aqueous phase was extracted with dichloromethane. The extract was dried (Na₂SO₄), filtered, and concentrated. The concentrate was purified by flash column chromatography on silica gel with 1% methanol in dichloromethane to provide 0.090 g (26%) of the desired product as a yellow solid. MS (DCI/NH₃) m/z 526 (M+H)⁺; ¹H NMR (300 MHz, CDCl₃) δ 8.65 (s, 1H), 8.22 (d, 1H), 7.62 (d, 1H), 7.47 (s, 1H), 4.80 (m, 1H), 3.97 (m, 1H), 3.93 (s, 3H), 3.64 (s, 3H), 2.82 (m, 2H), 2.13-1.72 (m, 4H), 1.53 (s, 9H), 1.20 (m, 2H), 0.98 (m, 2H).

### EXAMPLE 40B

### 7-(4-((tert-butoxycarbonyl)amino)-4,5,6,7-tetrahydro-1-benzothien-2-yl)-1-cyclopropyl-8-methoxy-4-oxo-1,4-dihydro-3-quinolinecarboxylic acid

Example 40A was processed as described in Example 2B to provide the desired product. MS (DCI/NH₃) m/z 511 (M+H)⁺; ¹H NMR (300 MHz, CDCl₃) δ 8.91 (s, 1H), 8.23 (d, 1H), 7.75 (d, 1H), 7.53 (s, 1H), 4.80 (m, 1H), 4.10 (m, 1H), 3.66 (s, 3H), 2.82 (m, 2H), 2.08 (m, 2H), 1.94 (m, 2H), 1.27 (m, 2H), 1.94 (m, 2H).

### EXAMPLE 40C

### 7-(4-amino-4,5,6,7-tetrahydro-1-benzothien-2-yl)-1-cyclopropyl-8-methoxy-4-oxo-1,4-dihydro-3-quinolinecarboxylic acid hydrochloride

A solution of Example 40B (0.087 g, 0.20 mmol) in dichloromethane (2 mL) at room temperature was treated with 4M HCl in dioxane (6 mL, 24.0 mmol), stirred for 2 hours, and concentrated. The concentrate was triturated with diethyl ether and filtered. The solid was rinsed with diethyl ether and dried to provide 0.059 g (76%) of the desired product as a yellow solid. mp 219-222° C.; MS (DCI/NH₃) m/z 411 (M+H)⁺; ¹H NMR (300 MHz, CD₃OD) δ 8.98 (s, 1H), 8.24 (d, 1H), 7.90 (d, 1H), 7.74 (s, 1H), 4.53 (m, 1H), 4.29 (m, 1H), 3.22 (s, 3H), 2.96 (m, 2H), 2.33-1.94 (m, 4H), 1.28 (m, 2H), 1.08 (m, 2H).

### EXAMPLE 41

### 1-cyclopropyl-7-(7-hydroxy-4,5,6,7-tetrahydro-1-benzothien-2-yl)-8-methoxy-4-oxo-1,4-dihydro-3-quinolinecarboxylic acid

### EXAMPLE 41A

### tert-butyl(dimethyl)(4,5,6,7-tetrahydro-1-benzothien-7-yloxy)silane

7-keto-4,5,6,7-tetrahydrothienanapthene (prepared by the method of MacDowell, et al. J. Heterocycl. Chem. 1965, 44-48) was processed as described in Example 35A to provide the desired product. MS (APCI(+)) m/z 267 (M+H)⁺; ¹H NMR (300 MHz, DMSO-d₆) 7.34 (d, 1H), 6.76 (d, 1H), 4.91 (m, 1H), 2.57-2.51 (br m, 2H), 1.98-1.88 (br m, 2H), 1.70-1.66 (m, 2H), 0.91 (s, 9H), 0.15 (s, 3H), 0.13 (s, 3H).

### EXAMPLE 41B

### tert-butyl(dimethyl)((2-(tributylstannyl)-4,5,6,7-tetrahydro-1-benzothien-7-yl)oxy)silane

Example 41A was processed as described in Example 1D to provide the desired product. MS (DCI/NH₃) m/z 427 (M-C₆H₁₅SiO)⁺; ¹H NMR (300 MHz, CDCl₃) 6.78 (s, 1H), 4.96 (m, 1H), 2.61-2.59 (m, 2H), 2.10-1.99 (br m, 2H), 1.79-1.74 (m, 2H), 1.54-0.94 (m, 36H), 0.18 (s, 3H), 0.15 (s, 3H).

### EXAMPLE 41C

### ethyl 7-(7-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylate

Ethyl 7-bromo-1-cyclopropyl-8-methoxy-4-oxo-1,4-dihydro-3-quinolonecarboxylate and Example 41A were processed as described in Example 1E to provide the desired product. MS (APCI(+)) m/z 554 (M+H)⁺; ¹H NMR (300 MHz, CDCl₃) 8.63 (s, 1H), 8.20 (d, 1H), 7.62 (d, 1H), 7.27 (s, 1H), 4.97 (m, 1H), 4.43 (q, 2H), 3.63 (s, 3H), 2.66-2.64 (br m, 2H), 2.07-2.04 (m, 2H), 1.85-1.79 (m, 2H), 1.42 (q, 3H), 0.97 (s, 9H), 0.22 (s, 3H), 0.18 (s, 3H).

### EXAMPLE 41D

### ethyl 4-cyclopropyl-6-(7-hydroxy-4,5,6,7-tetrahydro-1-benzothien-2-yl)-5-methoxy-1-oxo-1,4-dihydro-2-naphthalenecarboxylate

Example 41C was processed as described in Example 35E to provide the desired product. mp 183-185° C.; MS (APCI(+)) m/z 440 (M+H)⁺; ¹H NMR (300 MHz, CDCl₃) 8.56 (s, 1H), 7.96 (d, 1H), 7.78 (d, 1H), 7.45 (s, 1H), 5.44 (d, 1H), 4.74 (m, 1H), 4.23 (q, 2H), 4.08 (m, 1H), 3.64 (s, 3H), 2.59 (br s, 2H), 1.99-1.93 (m, 2H), 1.72-1.67 (m, 2H), 1.28 (t, 3H), 1.11 (m, 2H), 0.95 (s, 2H).

### EXAMPLE 41E

### 1-cyclopropyl-7-(7-hydroxy-4,5,6,7-tetrahydro-1-benzothien-2-yl)-8-methoxy-4-oxo-1,4-dihydro-3-quinolinecarboxylic acid

Example 41D was processed as described in Example 2B to provide the desired product. mp 238-240° C.; MS (APCI(+)) m/z 412 (M+H)⁺; ¹H NMR (300 MHz, CDCl₃) 8.79 (s, 1H), 8.11 (d, 1H), 7.98 (d, 1H), 7.54 (s, 1H), 5.47 (d, 1H), 4.75 (m, 1H), 4.25 (m, 1H), 3.68 (s, 3H), 2.60 (br s, 2H), 2.05-1.95 (br s, 2H), 1.72-1.68 (br m, 2H), 1.15 (m, 2H), 1.04 (m, 3H).

### EXAMPLE 42

### 1-cyclopropyl-7-(5-hydroxy-4,5,6,7-tetrahydro-1-benzothien-2-yl)-8-methoxy-4-oxo- 1,4-dihydro-3-quinolinecarboxylic acid

### EXAMPLE 42A

### tert-butl(dimethyl)(4,5,6,7-tetrahydro-1-benzothien-5-yloxy)silane

5-keto-4,5,6,7-tetrahydrothianapthene (prepared by the method of Padwa, et al. J. Org. Chem. 1989, 54, 299-308) was processed as described in Example 35A to provide the desired product. MS (DCI/NH₃) m/z 286 (M+NH₄)⁺; ¹H NMR (300 MHz, CDCl₃) 7.06 (d, 1H), 6.71 (d, 1H), 4.1 (m, 1H), 3.00-2.78 (m, 3H), 2.55 (m, 1H), 2.00 (m, 1H), 1.85 (m, 1H), 0.90 (s, 9H), 0.10 (s, 3H), 0.09 (s, 3H).

### EXAMPLE 42B

### tert-butyl(dimethyl)((2-(tributylstannyl)-4,5,6,7-tetrahydro-1-benzothien-5-yl)oxysilane

Example 42A was processed as described in Example 1D to provide the desired product. MS (DCI/NH₃) m/z 558 (M+H)⁺; ¹H NMR (300 MHz, CDCl₃) 6.77 (s, 1H), 4.1 (m, 1H), 3.00-2.78 (m, 3H), 2.55 (m, 1H), 2.00 (m, 1H), 1.85 (m, 1H), 1.65 -0.95 (m, 36), 0.09 (s, 3H), 0.08 (s, 3H).

### EXAMPLE 42C

### 7-(5-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylic acid

Ethyl 7-bromo-1-cyclopropyl-8-methoxy-4-oxo- 1,4-dihydro-3-quinolonecarboxylate and Example 42B were processed as described in Example 1E to provide the desired product. MS (DCI/NH₃) m/z 526 (M+H)⁺; ¹H NMR (300 MHz, CDCl₃) 14.77 (s, 1H), 8.90 (s, 1H), 8.22 (d, 1H), 7.75 (d, 1H), 7.32 (s, 1H), 4.23-4.05 (m, 2H), 3.67 (s, 3H), 3.05-2.80 (m, 3H), 2.63 (m, 1H), 2.00 (m, 1H), 1.90 (m, 1H), 1.26 (m, 2H), 1.05 (m, 2H), 0.91 (m, 9H), 0.12 (s, 3H), 0.11 (s, 3H).

### EXAMPLE 42D

### 1-cyclopropyl-7-(5-hydroxy-4,5,6,7-tetrahydro-1-benzothien-2-yl)-8-methoxy-4-oxo-1,4-dihydro-3-quinolinecarboxvlic acid

Example 42C was processed as described in Example 35E to provide the desired product. mp 232-233° C.; MS (DCI/NH₃) m/z 412 (M+H)⁺; ¹H NMR (300 MHz, DMSO-d₆) 8.78 (s, 1H), 8.10 (d, 1H), 7.98 (d, 1H), 7.55 (s, 1H), 4.84 (d, 1H), 4.25 (m, 1H), 4.00 (m, 2H), 3.67 (s, 3H), 3.00-2.70 (m, 4H), 1.95 (m, 1H), 1.80 (m, 1H), 1.20-1.00 (m, 4H).

### EXAMPLE 43

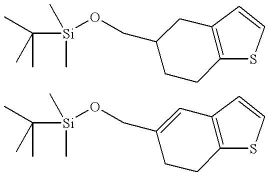

### 1-cyclopropyl-7-(5-(hydroxymethyl)-4,5,6,7-tetrahydro-1-benzothien-2-yl)-8-methoxy-4-oxo-1,4-dihydro-3-quinolinecarboxylic acid

### EXAMPLE 43A and EXAMPLE 44A

### tert-butyl(dimethyl)(4,5,6,7-tetrahydro-1-benzothien-5-ylmethoxy)silane and

### tert-butyl(6,7-dihydro-1-benzothien-5-ylmethoxy)dimethylsilane

A solution of methyl 6,7-dihydrobenzothiophene-5-carboxylate (5.0 g, 25.77 mmol) (prepared by the method of Amemiya, et al. J. Med. Chem. 1989, 32, 1265-72) and 10% Pd/C (3.5 g) in absolute ethanol (200 mL) was stirred at room temperature under hydrogen for 6.5 hours and filtered. The filtrate was concentrated to provide 3.5 g (69%) of a first oil comprising one part methyl 4,5,6,7-tetrahydrobenzothiophene-5-carboxylate and two parts methyl 6,7-dihydrobenzothiophene-5-carboxylate.

A solution of first oil (3.5 g, 17.86 mmol) in diethyl ether (38 ml) at 5° C. was treated with lithium aluminum hydride powder (0.68 g, 17.86 mmol), stirred for 10 minutes at 5° C. and for 30 minutes at room temperature, and quenched with Na₂SO₄.10 H₂O (6.56 g), and filtered. The filtrate was concentrated to provide 3.0 g (100%) of a second oil comprising 5-hydroxymethyl 4,5,6,7-tetrahydrobenzothiophene and 5-hydroxymethyl 6,7-dihydrobenzothiophene.

A solution of the second oil (3.0 g, 17.86 mmol), tert-butyldimethylchlorosilane (10.7 g, 71.44 mmol), and imdazole (4.9 g, 71.44 mmol) in DMF (20 ml) at room temperature was stirred for 17 hours and distilled at 35° C. under high vacuum to remove excess tert-butyldimethylchlorosilane. The residue was partitioned between diethyl ether and water, and the aqueous phase extracted with diethyl ether. The combined extracts were washed with 1M HCl and brine, dried (MgSO₄), filtered, and concentrated. The concentrate was purified by flash column chromatography on silica gel with hexanes then 2% ethyl acetate in hexanes to provide the following desired products as oils:

Example 43A: tert-butyl(dimethyl)(4,5,6,7-tetrahydro-1-benzothien-5-ylmethoxy)silane, 1.0 g, (20%). MS (DCI/NH₃) m/z 283 (M+H)⁺; ¹H NMR (300 MHz, CDCl₃) 7.04 (d, 1H), 6.74 (d, 1H), 3.58 (dd, 2H), 2.90-2.69 (m, 4H), 2.34-2.24 (m, 1H), 2.06-1.89 (m, 2H), 0.90 (s, 9H), 0.05 (s, 6H). and

Example 44A: tert-butyl(6,7-dihydro-1-benzothien-5-ylmethoxy)dimethylsilane, 2.3 g, (46%). MS (DCI/NH₃) m/z 298 (M+NH₄)⁺; ¹H NMR (300 MHz, CDCl₃) 7.01 (d, 1H), 6.83 (d, 1H), 6.41 (m, 1H), 4.22 (br s, 2H), 2.90 (t, 2H), 2.36 (t, 2H), 0.94 (s, 9H), 0.10 (s, 6H).

### EXAMPLE 43B

### tert-butyl(dimethyl)((2-(tributylstannyl)-4,5,6,7-tetrahydro-1-benzothien-5-yl)methoxy)silane

Example 43A was processed as described in Example 1D to provide the desired product. MS (DCI/NH₃) m/z 573 (M+H)⁺; ¹H NMR (300 MHz, CDCl₃) 6.81 (s, 1H), 3.59 (dd, 2H), 2.95-2.70 (m, 4H), 2.36-2.27 (m, 1H), 2.05-1.90 (m, 2H), 1.62-0.90 (m, 36H), 0.05 (s, 6H).

### EXAMPLE 43C

### ethyl 7-(5-(((tert-butyl(dimethyl)silyl)oxy)methyl)-4,5,6,7-tetrahydro-1-benzothien-2-yl)-1-cyclopropyl-8-methoxy-4-oxo-1 4-dihydro-3-quinolinecarboxylate

Ethyl 7-bromo-1-cyclopropyl-8-methoxy-4-oxo-1,4-dihydro-3-quinolonecarboxylate and Example 43B were processed as described in Example 1E to provide the desired product. MS (DCI/NH₃) m/z 568 (M+H)⁺; ¹H NMR (300 MHz, CDCl₃) 8.63 (s, 1H), 8.20 (d, 1H), 7.62 (d, 1H), 7.29 (s, 1H), 4.40 (q, 2H), 3.97 (m, 1H), 3.65 (s, 3H), 3.63 (d, 2H), 2.96-2.75 (m, 3H), 2.41-2.30 (m, 1H), 2.11- 1.95 (m, 2H), 1.63-1.51 (m, 1H), 1.41 (t, 3H), 1.18 (m, 2H), 0.97 (m, 2H), 0.93 (s, 9H), 0.08 (s, 6H).

### EXAMPLE 43D

### ethyl 1-cyclopropyl-7-(5-(hydroxamethyl)-4,5,6,7-tetrahydro-1-benzothien-2-yl)-8-methoxy-4-oxo-1 4-dihydro-3-quinolinecarboxylate

Example 43C was processed as described in Example 35E to provide the desired product. MS (DCI/NH₃) m/z 454 (M+H)⁺; ¹H NMR (300 MHz, CDCl₃) 8.63 (s, 1H), 8.21 (d, 1H), 7.62 (d, 1H), 7.29 (s, 1H), 4.40 (q, 2H), 3.97 (m, 1H), 3.69 (m, 2H), 3.65 (s, 3H), 2.98-2.78 (m, 3H), 2.39 (m, 1H), 2.17-2.01 (m, 2H), 1.66-1.54 (m, 1H), 1.41 (t, 3H), 1.19 (m, 2H), 0.98 (m, 2H).

### EXAMPLE 43E

### 1-cyclopropyl-7-(5-(hydroxymethyl)-4,5,6,7-tetrahydro-1-benzothien-2-yl)-8-methoxy-4-oxo-1,4-dihydro-3-quinolinecarboxylic acid

Example 43C was processed as described in Example 2B to provide the desired product. mp 218-219° C.; MS (ESI(+)) m/z 412 (M+H)⁺; ¹H NMR (300 MHz, DMSO-d₆) 14.95 (s, 1H), 8.78 (s, 1H), 8.09 (d, 1H), 7.99 (d, 1H), 7.57 (s, 1H), 4.59 (t, 1H), 4.25 (m, 1H), 3.68 (s, 3H), 3.42 (t, 2H), 2.92-2.71 (m, 3H), 2.33-2.24 (m, 1H), 2.02 (m, 1H), 1.86 (m, 1H), 1.45 (m, 1H), 1.16 (m, 2H), 1.04 (m, 2H).

### EXAMPLE 44

### 1-cyclopropyl-7-(5-(hydroxymethyl)-6,7-dihydro-1-benzothien-2-yl)-8-methoxy-4-oxo-1,4-dihydro-3-quinolinecarboxylic acid

### EXAMPLE 44B

### tert-butyl(dimethyl)((2-(tributylstannyl)-6,7-dihydro-1-benzothien-5-yl)methoxy)silane

Example 44A was processed as described in Example 1D to provide the desired product. MS (DCI/NH₃) m/z 571 (M+H)⁺; 1H NMR (300 MHz, CDCl₃) 6.88 (s, 1H), 6.45 (m, 11H), 4.22 (br s, 2H), 2.93 (t, 2H), 2.35 (t, 2H), 1.55-0.90 (m, 36H), 0.10 (s, 6H).

### EXAMPLE 44C

### ethyl 7-(5-(((tert-butyl(dimethyl)silyloxymethyl)-6,7-dihydro-1-benzothien-2-yl)-1-cyclopropyl-8-methoxy-4-oxo-1,4-dihydro-3-quinolinecarboxylate

Ethyl 7-bromo-1-cyclopropyl-8-methoxy-4-oxo-1,4-dihydro-3-quinolonecarboxylate and Example 44B were processed as described in Example 314729E to provide the desired product. MS (DCI/NH₃) m/z 566 (M+H)⁺; ¹H NMR (300 MHz, CDCl₃) 8.63 (s, 1H), 8.21 (d, 1H), 7.63 (d, 1H), 7.36 (s, 1H), 6.46 (m, 1H), 4.40 (q, 2H), 4.25 (br s, 2H), 3.98 (m, 1H), 3.64 (s, 3H), 2.97 (t, 2H), 2.41 (t, 2H), 1.41 (t, 3H), 1.19 (m, 2H), 0.97 (m, 2H), 0.95 (s, 9H), 0.12 (s, 6H).

### EXAMPLE 44D

### ethyl 1-cyclopropyl-7-(5-(hydroxymethyl)-6,7-dihydro-1-benzothien-2-yl)-8-methoxy-4-oxo-1,4-dihydro-3-quinolinecarboxylate

Example 44C was processed as described in Example 35E to provide the desired product. MS (DCI/NH₃) m/z 452 (M+H)⁺; ¹H NMR (300 MHz, CDCl₃) 8.63 (s, 1H), 8.22 (d, 1H), 7.62 (d, 1H), 7.36 (s, 1H), 6.49 (m, 1H), 4.85 (d, 1H), 4.40 (q, 2H), 4.26 (d, 2H), 3.97 (m, 1H), 3.64 (s, 3H), 2.99 (t, 2H), 2.49 (t, 2H), 1.42 (t, 3H), 1.19 (m, 2H), 0.98 (m, 2H).

### EXAMPLE 44E

### 1-cyclopropyl-7-(5-(hydroxvmethyl)-6,7-dihydro-1-benzothien-2-yl)-8-methoxy-4-oxo-1,4-dihydro-3-quinolinecarboxylic acid

Example 44D was processed as described in Example 2B to provide the desired product. mp 205-206° C.; MS (ESI(+)) m/z 424 (M+H)⁺; ¹H NMR (300 MHz, CDCl₃) 8.90 (s, 1H), 8.23 (d, 1H), 7.76 (d, 1H), 7.42 (s, 1H), 6.51 (m, 1H), 4.86 (s, 1H), 4.27 (s, 2H), 4.11 (m, 1H), 3.68 (s, 3H), 3.01 (t, 2H), 2.50 (t, 2H), 1.28 (m, 2H), 1.06 (m, 2H).

### EXAMPLE 45

### 1-cyclopropyl-7-(5-hydroxy-5,6-dihydro-4H-cyclopentarblthien-2-yl)-8-methoxM-4-oxo-1,4-dihydro-3-quinolinecarboxylic acid

### EXAMPLE 45A

### 1-diazo-3-(2-thienyl)acetone

A solution of 2-(2-thiophenyl)-acetic acid (13.56 g, 84.4 mmol) and 3 drops of DMF in 1:1 dichloromethane-hexanes (340 mL) was treated dropwise over 20 minutes with oxalyl chloride (9.20 mL, 105.5 mmol), stirred at ambient temperature for 3 hours, and concentrated to provide 4.2 g (ca. 100%) of the acid chloride as a brown oil. A solution of the acid chloride in diethyl ether (200 mL) was treated with a solution of diazomethane (ca. 240 mmol) in ether (500 mL) (Generated according to the method of Hudlicky, J. Org. Chem. 1980, 45, 5377 from Diazald (51.7 g, 241.1 mmol) and KOH (20g, 314 mmol)). The solution sat with occasional swirling for 30 minutes then was treated with several drops of acetic acid to decompose the excess diazomethane, washed with saturated NaHCO₃ and brine, dried (Na₂SO₄), filtered, and concentrated. The concentrate was purified by flash column chromatography on silica gel with hexanes then with 10% ethyl acetate in hexanes to provide 11.0 g (78%) of the desired product as an off-white solid. MS (ESI(+)) m/z 167 (M+H)⁺ and 184 (M+NH₄)⁺; ¹H NMR (300 MHz, CDCl₃) 7.24 (dd, 1H), 6.99 (dd, 1H), 6.92 (m, 1H), 5.25 (br s, 1H), 3.81 (br s, 2H).

### EXAMPLE 45B

### 4,6-dihydro-5H-cyclopenta[b]thiophen-5-one

A solution of Example 45A (11.0 g, 66.21 mmol) in dichloromethane (1.55 mL) at room temperature was treated with dirhodium tetraacetate (125 mg), stirred for 3 hours, and concentrated. The residue was purified by flash column chromatography on silica gel with hexanes then 5% ethyl acetate in hexanes to provide 2.83 g (31%) of the desired product as a white solid. ¹H NMR (300 MHz, CDCl₃) 7.26 (ddd, 1H), 6.97 (d, 1H), 3.54 (s, 2H), 3.42 (s, 2H).

### EXAMPLE 45C

### 5,6-dihydro-4H-cyclopenta[b]thiophen-5-ol

A solution of Example 45B (1.69 g, 12.23 mmol) in THF (61 mL) at 0° C. was treated dropwise over 10 minutes with 1M solution of lithium aluminum hydride in THF (6.1 mL, 6.1 mmnol), stirred for 30 minutes, warmed to room temperature, recooled to 0° C., treated sequentially with ethyl acetate (50 mL) saturated sodium potassium tartrate (75 mL), and ethyl acetate. The layers were separated, and the organic layer was washed with water and brine, dried (Na₂SO₄), filtered, and concentrated to provide 1.61 g (94%) of the desired product as a tan solid. MS (DCI/NH₃) m/z 141 (M+H)⁺ and 158 (M+NH₄)⁺, ¹H NMR (300 MHz, CDCl₃) 7.19 (dd, 1H), 6.83 (d, 1H), 4.99 (m, 1H), 3.26 (dd, 1H), 3.12 (dd, 1H), 2.86 (ddd, 1H), 2.71 (dd, 1H).

### EXAMPLE 45D

### tert-butyl(5,6-dihydro-4H-cyclopenta[b]thien-5-yloxy)dimethylsilane

A solution of Example 45C (2.15 g, 15.34 mmol), N, N-diisopropylethylamine (3.97 g, 30.68 mmol), and 4-(N,N-dimethylamino)pyridine (0.469 g, 3.83 mmol) in dichloromethane (51 mL) at room temperature was treated with the tert-butyldimethylchlorosilane (2.78 g, 18.40 mmol), stirring for 24 hours, diluted with dichloromethane, extracted with water, saturated ammonium chloride solution, and saturated sodium bicarbonate solution, dried (Na₂SO₄), filtered, and concentrated. The concentrate was purified by flash column chromatography on silica gel with hexanes then with ethyl acetate in hexanes to provide 3.8 g (97%) of the desired product as a colorless oil. MS (ESI(+)) m/z 252 (M+H)⁺and 272 (M+NH₄)⁺; ¹H NMR (300 MHz, CDCl₃) 7.14 (dt, 1H), 6.78 (d, 1H), 4.98 (m, 1H), 3.07 (dd, 1H), 3.04 (ddd, 1H), 2.72 (dddd, 1H), 2.67 (dddd, 1H), 0.91 (s, 9H), 0.10 (s, 6H).

### EXAMPLE 45E

### tert-butyl(dimethyl)((2-(tributylstannyl)-5,6-dihydro-4H-cyclopenta[b]thien-5-yl)oxy)silane

Example 45D was processed as described in Example ID to provide the desired product. ¹H NMR (300 MHz, CDCl₃) 6.84 (s, 1H), 5.00 (m, 1H), 3.20 (dd, 1H), 3.05 (dd, 1H), 2.83 (dddd, 1H), 2.68 (dddd, 1H), 1.58 (m, 6H), 1.37 (m, 6H), 1.24 (m, 6H), 0.92 (m, 18H), 0.10 (s, 6H).

### EXAMPLE 45F

### ethyl 7-(5-((tert-butyl(dimethyl)silyl)oxy)-5,6-dihydro-4H-cyclopenta[b]thien-2-yl)-1-cyclopropyl-8-methoxy-4-oxo-1,4-dihydro-3-quinolinecarboxylate

Ethyl 7-bromo-1-cyclopropyl-8-methoxy-4-oxo-1,4-dihydro-3-quinolonecarboxylate and Example 45E were processed as described in Example 1E to provide the desired product. MS (ESI(+)) m/z 540 (M+H)⁺; ¹H NMR (300 MHz, CDCl₃) 8.18 (d, 1H), 7.58 (d, 1H), 7.31 (s, 1H), 5.00 (m, 1H), 4.37 (q, 2H), 3.95 (m, 1H), 3.61 (s, 3H), 3.23 (dd, 1H), 3.08 (dd, 1H), 2.88 (dd, 1H), 2.72 (dd, 1H), 1.39 (t, 3H), 1.16 (m, 2H), 0.95 (m, 2H), 0.90 (s, 9H), 0.10 (s, 6H).

### EXAMPLE 45G

### ethyl 1-cyclopropyl-7-(5-hydroxy-5,6-dihydro-4H-cyclopenta[b]thien-2-yl)-8-methoy-4-oxo-1,4-dihydro-3-quinolinecarboxylate

A solution of Example 45F (0.777 g, 1.43 mmol) in THF (30 mL) at 0° C. was treated with pyridinium hydrofluoride (15 mL) portionwise over 1 hour, warmed to room temperature diluted with water and ethyl acetate. The layers were separated, and the organic layer was washed with water and saturated sodium bicarbonate solution, dried (Na₂SO₄), filtered, and concentrated. The residue was purified by flash column chromatography on silica gel with hexanes then with ethyl acetate in hexanes to provide 472 mg (77%) of the desired product as an off-white solid. mp 161-163° C.; MS (ESI(+)) m/z 426 (M+H)⁺; ¹H NMR (300 MHz, CDCl₃) 8.63 (s, 1H), 8.20 (d, 1H), 7.59 (d, 1H), 7.37 (s, 1H), 5.05 (m, 1H), 4.40 (q, 2H), 3.97 (m, 1H), 3.64 (s, 3H), 3.34 (dd, 1H), 3.18 (dd, 1H), 2.96 (dd, 1H), 2.70 (dd, 1H), 2.10 (br d, 1H), 1.41 (t, 3H), 1.19 (m, 2H), 0.98 (m, 2H).

### EXAMPLE 45H

### 1-cyclopropyl-7-(5-hydroxy-5,6-dihydro-4H-cyclopenta[b]thien-2- yl)-8-methoxy-4-oxo-1,4-dihydro-3-quinolinecarboxvlic acid

Example 45G was processed as described in Example 2B to provide the desired product. mp 254-255° C.; MS (ESI(+)) m/z 398 (M+H)⁺; ¹H NMR (300 MHz, CDCl₃) 14.75 (s, 1H), 8.90 (s, 1H), 8.22 (d, 1H), 7.74 (d, 1H), 7.44 (s, 1H), 5.06 (m, 1H), 4.11 (m, 1H), 3.67 (s, 3H), 3.36 (dd, 1H), 3.21 (dd, 1H), 2.98 (dd, 1H), 2.81 (dd, 1H), 1.26 (m, 2H), 1.06 (m, 2H).

### EXAMPLE 46

### 1-cyclopropyl-8-methoxy-7-(4-methoxy-5,6-dihydro-4H-cyclopenta[b]thien-2-yl)-4-oxo-1,4-dihydro-3-quinolinecarboxylic acid

### EXAMPLE 46A

### 4-methoxy-5,6-dihydro-4H-cyclopenta[b]thiophene

A solution of 5,6-dihydrocyclopenta[b]thiophene-4-one (1.25 g, 9.06 mmol, prepared by the method of Balenkova, E. S., et al. Tetrahedron Lett. 1996, 37, 4199) in methanol at 0° C. was treated portionwise with NaBH₄, stirred at room temperature for 45 minutes, quenched with 1M HCl, and extracted 3 times with ethyl acetate. The combined extracts were dried (Na₂SO₄), filtered, and concentrated to provide an oil which was purified by flash column chromatography on silica gel with 10% ethyl acetate in hexanes to provide 0.84 g (60%) of the desired product as a clear oil. MS (DCI/NH₃) m/z 172 (M+NH₄)⁺; ¹H NMR (300 MHz, DMSO-d₆) δ 7.38 (d, 1H), 7.00 (d, 1H), 4.69 (dd, 1H), 3.25 (s, 3H), 3.05-2.85 (br m, 1H), 2.82-2.60 (br m, 2H), 2.35-2.22 (br m, 1H).

### EXAMPLE 46B

### methyl 2-(tributylstannyl)-5,6-dihydro-4H-cyclopenta[b]thien-4-yl ether

Example 46A was processed as described in Example 1D to provide the desired product. MS (DCI/NH₃) m/z 412 (M-CH₃OH)⁺; ¹H NMR (300 MHz, CDCl₃) 6.99 (s, 1H), 4.76 (m, 1H), 3.38 (s, 3H), 3.05 (m, 1H), 2.78 (m, 2H), 2.48 (m, 1H), 1.55 (m, 6H), 1.33 (m, 6H), 1.07 (m, 6H), 0.89 (t, 9H).

### EXAMPLE 46C

### 1-cyclopropyl-8-methoxy-7-(4-methoxy-5,6-dihydro-4H-cyclopenta[b]thien-2-yl)-4-oxo-1,4-dihydro-3-quinolinecarboxylic acid

Ethyl 7-bromo-1-cyclopropyl-8-methoxy-4-oxo-1,4-dihydro-3-quinolonecarboxylate and Example 46B were processed as described in Example 1E to provide the desired product. MS (DCI/NH₃) m/z 412 (M+H)⁺; ¹H NMR (300 MHz, DMSO-d₆) δ 14.92 (br s, 1H), 8.80 (s, 1H), 8.12 (d, 1H), 8.04 (d, 1H), 7.80 (s, 1H), 4.79 (dd, 1H), 4.30-4.20 (m, 1H), 3.69 (s, 3H), 3.14-3.00 (m, 1H), 2.95-2.80 (m, 1H), 2.78-2.62 (m, 1H), 2.38-2.34 (m, 1H), 1.20-1.00 (br m, 4H).

### EXAMPLE 47

### 1-cyclopropyl-7-(6-hydroxy-4,5,6,7-tetrahydro-1-benzothien-2-yl)-8-methoxy-4-oxo-1,4-dihydro-3-quinolinecarboxylic acid

### EXAMPLE 47A

### tert-butyl(dimethyl)(4,5,6,7-tetrahydro-1-benzothien-6-yloxy)silane

4,5-Dihydrobenzo[b]thiophene-6(7H)-one (prepared by the method of Padwa, A., et al. J. Org. Chem. 1989, 54, 299) was processed according to Example 35A to provide the desired product. MS (DCI/NH₃) m/z 269 (M+H)⁺; ¹H NMR (300 MHz, DMSO-d₆) δ 7.13 (d, 1H), 6.68 (d, 1H), 4.13-4.03 (m, 1H), 2.89 (dd, 1H), 2.67-2.43 (m, 3H), 1.81-1.54 (m, 2H), 0.90 (s, 9H), 0.12 (s, 3H), 0.11 (s, 3H).

### EXAMPLE 47B

### tert-butyl(dimethyl)((2-(tributylstannyl)-4,5,6,7-tetrahydro-1-benzothien-6-yl)oxysilane

Example 47A was processed as described in Example 1D to provide the desired product. ¹H NMR (300 MHz, DMSO-d₆) δ 6.79 (s, 1H), 4.18-4.10 (m, 1H), 3.05-2.93 (m, 1H), 2.80-2.53 (m, 3H), 1.90-1.60 (m, 2H), 1.60-1.45 (m, 6H), 1.40-1.20 (m, 6H), 1.10-0.97 (m, 6H), 0.99 (s, 3H), 0.90-0.80 (m, 18H), 0.10 (s, 3H).

### EXAMPLE 47C

### 7-(6-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxvlic acid

Ethyl 7-bromo-1-cyclopropyl-8-methoxy-4-oxo-1,4-dihydro-3-quinolonecarboxylate and Example 47B were processed as described in Example 1E to provide the desired product. MS (DCI/NH₃) m/z 526 (M+H)⁺; ¹H NMR (300 MHz, DMSO-d₆) δ 14.83 (s, 1H), 8.68 (s, 1H), 8.00 (d, 1H), 7.87 (d, 1H), 7.46 (s, 1H), 4.20-4.08 (m, 2H), 3.95 (dd, 1H), 3.57 (s, 3H), 2.70-2.53 (m, 5H), 1.85-1.60 (m, 4H), 1.10-0.86 (m, 4H), 0.99 (s, 3H), 0.77 (s, 9H), 0.10 (s, 3H).

### EXAMPLE 47D

### 1-cyclopropyl-7-(6-hydroxy-4,5,6,7-tetrahydro-1-benzothien-2-yl)-8-methoxy-4-oxo-1,4-dihydro-3-quinolinecarboxylic acid

Example 47C was processed as described in Example 35E to provide the desired product. MS (DCI/NH₃) m/z 412 (M+H)⁺; ¹H NMR (300 MHz, DMSO-d₆) δ 8.78 (s, 1H), 8.09 (d, 1H), 7.96 (d, 1H), 7.55 (s, 1H), 4.93 (d, 1H), 4.30-4.19 (m, 1H), 4.10-3.97 (m, 1H), 3.67 (s, 3H), 3.03 (dd, 1H), 2.85-2.55 (m, 3H), 1.95-1.83 (m, 1H), 1.80-1.68 (m, 1H), 1.20-1.00 (m, 4H).

### EXAMPLE 48

### 1-cyclopropyl-6-fluoro-4-oxo-7-(4-oxo-4,5,6,7-tetrahydro-1-benzothien-2-yl)-1,4-dihydro[1,8]naphthyridine-3-carboxylic acid

### EXAMPLE 48A

### tert-butyl(6,7-dihydro-1-benzothien-4-yloxy)dimethylsilane

A solution of 1.0M lithium bis(trimethylsilyl)amide in THF (26.4 mL, 26.4 mmol) at −40° C. was treated dropwise with a solution of 4-keto-4,5,6,7-tetrahydrothianaphthene (3.64 g, 24.0 mmol) in THF (30 mL), stirred for 2 hours, and treated dropwise with a solution of t-butyldimethylsilyl chloride (3.72 g, 24.8 mmol) in THF (30 mL). After 1 hour at −40° C., the reaction mixture warmed to room temperature, stirred for 18 hours, poured into cold 10% aqueous NH₄Cl. The layers were separated, and the aqueous layer was extracted with dichloromethane. The combined extracts were dried (Na₂SO₄) filtered, and concentrated to provide 6.07 g (95%) of the desired product as an oil. MS (APCI(+)) mn/z 267 (M+H)⁺; ¹H NMR (300 MHz, CDCl₃) 7.02 (d, 1H), 6.97 (d, 1H), 4.85 (dd, 1H), 2.80 (dd, 2H), 2.62 (m, 2H), 1.00 (s, 9H), 0.20 (s, 6H).

### EXAMPLE 48B

### tert-butyl(dimethyl)((2-(tributylstannyl)-6,7-dihydro-1-benzothien-4-yl)oxysilane

Example 48B was processed as described in Example 1D to provide the desired product. MS (APCI(+)) m/z 556 (M+H)⁺; ¹H NMR (CDCl₃) 7.10 (s, 1H), 5.83 (dd, 1H), 2.82 (dd, 2H), 2.40 (m, 2H), 1.50-1.02 (m, 18H), 1.00 (s, 9H), 0.90 (t, 9H), 0.20 (s, 6H).

### EXAMPLE 48C

### ethyl 1-cyclopropyl-6-fluoro-4-oxo-7-(4-oxo-4,5,6,7-tetrahydro-1-benzothien-2-yl)-1,4-dihydro[1,8]naphthyridine-3-carboxylate

Ethyl 7-chloro-1-cyclopropyl-6-fluoro-4-oxo- 1,4-dihydro[1,8]napthyridine-3-carboxylate and Example 48B were processed as described in Example 1E to provide the desired product. MS (APCI(+)) m/z 426 (M+H)⁺; ¹H NMR (300 MHz, CDCl₃) 8.68 (s, 1H), 8.43 (d, 1H), 8.25 (d, 1H), 4.41 (q, 2H), 3.68 (m, 1H), 3.12 (dd, 2H), 2.63 (dd, 2H), 2.29 (m, 2H), 1.41 (t, 3H), 1.10 (m, 2H), 0.90 (m, 2H).

### EXAMPLE 48D

### 1-cyclopropyl-6-fluoro-4-oxo-7-(4-oxo-4,5,6,7-tetrahydro-1-benzothien-2-yl)-1,4-dihydro[1,8]naphthyridine-3-carboxylic acid

A solution of Example 48C (0.155 g, 0.37 mmol) in ethanol (3 mL) was treated with 1M HCl (3.7 mL, 3.7 mmol), heated at 90° C. for 5 hours, and concentrated. The residue was dissolved in dichloromethane, washed with water, dried (Na₂SO₄), filtered, and concentrated to provide 0.065 g (44%) of the desired product as a pale yellow solid. mp 298-299° C. (decomp.); MS (APCI(+)) m/z 399 (M+H)⁺; ¹H NMR (300 MHz, CDCl₃) 8.90 (s, 1H), 8.46 (d, 1H), 8.32 (d, 1H), 3.82 (dt, 1H), 3.13 (dd, 2H), 2.62 (dd, 2H), 2.30 (dt, 2H), 1.42 (m, 2H), 1.15 (m, 2H).

### EXAMPLE 49

### 1-cyclopropyl-6-fluoro-7-((4E)-4-(hydroxyimino)-4,5,6,7-tetrahydro-1-benzothien-2-yl)-4-oxo-1,4-dihydro[1,8]naphthyridine-3-carboxylic acid

Example 48D (0.250 g, 0.62 mmol) was suspended in 2:1 ethanol/THF (8 mL), and treated with hydroxylamine hydrochloride (0.161 g, 2.50 mmol) and sodium bicarbonate (0.218 g, 2.60 mmol) in water (3 mL), heated at 75° C. for 24 hours, cooled to room temperature, and diluted with to form a precipitate. The precipitate was collected by filtration, triturated in iso-propanol, filtered, and dried to provide 0.220 g (85%) of the desired product as an off-white solid. mp>300° C.; MS (APCI(−)) m/z 412 (M−H)⁺; ¹H NMR (300 MHz, DMSO-d₆) 13.5 (br s, 1H), 11.60 (s, 1H), 8.65 (s, 1H), 8.35 (d, 1H), 8.05 (d, 1H), 3.70 (m, 1H), 2.93 (dd, 2H), 2.67 (dd, 2H), 1.98 (m, 2H), 1.28 (m, 2H), 1.08 (m, 2H).

### EXAMPLE 50

### 1-cyclopropyl-8-methoxy-4-oxo-7-(4-oxo-4,5,6,7-tetrahydro-1-benzothien-2-yl)-1,4-dihydro-3-quinolinecarboxylic acid

### EXAMPLE 50A

### ethyl 1-cyclopropyl-8-methoxy-4-oxo-7-(4-oxo-4,5,6,7-tetrahydro-1-benzothien-2-yl)-1,4-dihydro-3-guinolinecarboxylate

Ethyl 7-bromo-1-cyclopropyl-8-methoxy-4-oxo-1,4-dihydro-3 -quinolonecarboxylate and Example 48B were processed as described in Example 1E to provide the desired product. MS (APCI(+)) m/z 438 (M+H)⁺; ¹H NMR (300 MHz, CDCl₃) 8.65 (s, 1H), 8.36 (d, 1H), 7.90 (s, 1H), 7.63 (d, 1H), 4.40 (q, 2H), 3.97 (m, 1H), 3.63 (s, 3H), 3.10 (dd, 2H), 2.62 (dd, 2H), 2.28 (m, 2H), 1.41 (t, 3H), 1.20 (m, 2H), 0.98 (m, 2H).

### EXAMPLE 50B

### 1-cyclopropyl-8-methoxy-4-oxo-7-(4-oxo-4,5,6,7-tetrahydro-1-benzothien-2-yl)-1,4-dihydro-3-quinolinecarboxylic acid

Example 50A was processed as described in Example 48D to provide the desired product. mp 266-267° C.; MS (APCI(+)) m/z 410 (M+H)⁺; MS (APCI(−)) 408 (M−H)⁻; ¹H NMR (300 MHz, CDCl₃) 14.65 (s, 1H), 8.92 (s, 1H), 8.27 (d, 1H), 7.95 (s, 1H), 7.77 (d, 1H), 4.10 (m, 1H), 3.68 (s, 3H), 3.12 (dd, 2H), 2.63 (dd, 2H), 2.30 (m, 2H), 1.30 (m, 2H), 1.04 (m, 2H).

### EXAMPLE 51

### 1-cyclopropyl-7-((4E)-4-(hydroxyimino)-4,5,6,7-tetrahydro-1-benzothien-2-yl-8-methoxy-4-oxo-1,4-dihydro-3-quinolinecarboxylic acid

Example 50B was processed as described in Example 49 to provide the desired product. mp 296-297° C.; MS (APCI(+)) m/z 425 (M+H)⁺; ¹H NMR (300 MHz, DMSO-d₆) 12.25 (s, 1H), 10.85 (s, 1H), 8.80 (s, 1H), 8.13 (d, 1H), 8.00 (d, 1H), 7.91 (s, 1H), 4.25 (m, 1H), 3.68 (s, 3H), 2.90 (dd, 2H), 2.67 (dd, 2H), 1.93 (m, 2H), 1.16 (m, 2H), 1.05 (m, 2H).

### EXAMPLE 52

### 1-cyclopropyl-8-diflouromethoxy-7-(7-methyl-4,5,6,7-tetrahydrotheino(2,3-c)pyrindin-2-yl)-4-oxo-1,4-dihydro-3-quinolinecarboxylic acid hydrochloride

Ethyl 7-bromo-1-cyclopropyl-8-difluoromethoxy-4-oxo-1,4-dihydro-3-quinolinecarboxylate and Example 21D were processed as described in Example 1E to provide the desired product. mp 222-225° C.; MS (DCI/NH₃) m/z 447 (M+H)⁺; ¹H NMR (300 MHz, CD₃OD) δ 9.01 (s, 1H), 8.40 (d, 1H), 7.90 (d, 1H), 7.55 (s, 1H), 6.62 (dd, 1H), 4.86 (m, 1H), 4.27 (m, 1H), 3.74 (m, 1H), 3.52 (m, 1H), 3.10 (m, 2H), 1.76 (d, 3H), 1.32 (m, 2H), 1.07 (m, 2H).

### EXAMPLE 53

### 1-cyclopropyl-8-methoxy-4-oxo-7-(5-oxo-4,5,6,7-tetrahydro-1-benzothien-2-yl)-1,4-dihydro-3-quinolinecarboxylic acid

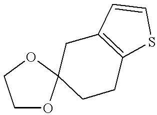

### EXAMPLE 53A

A solution of 5-keto-4,5,6,7-tetrahydrothianapthene (prepared by the method of Padwa, et al. J. Org. Chem. 1989, 54, 299-308.) (3.00g, 19.7 mmol), ethylene glycol (2.50 g, 40.0 mmol), and p-toluenesulfonic acid (0.380 g, 2.00 mmol) in toluene was heated to 100° C. overnight. After cooling, the mixture was partitioned between ethyl acetate and water. The organic phase was washed with saturated aqueous sodium bicarbonate, water, and brine, dried (Na₂SO₄), and concentrated. The resulting residue was purified by chromatography on silica gel eluting with 10% ethyl acetate in hexane to provide the desired product (3.44 g, 89%) as a yellow oil.

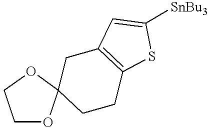

### EXAMPLE 53B

The desired product was prepared by substituting Example 53A for Example 1C in Example 1D.

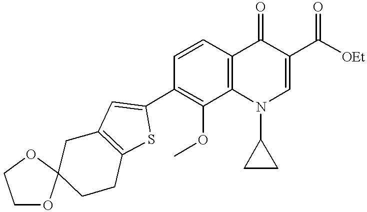

### EXAMPLE 53C

The desired product was prepared by substituting Example 53B and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E.

### EXAMPLE 53D

### 1-cyclopropyl-8-methoxy-4-oxo-7-(5-oxo-4,5,6,7-tetrahydro-1-benzothien-2-yl)-1,4-dihydro-3-quinolinecarboxylic acid

The desired product was prepared by substituting Example 53C for Example 48C in Example 48D. MS (APCI) m/z 410 (M+1)⁺; ¹H NMR (300 MHz, CDCl₃) δ 14.68 (s, 1H), 8.92 (s, 1H), 8.26 (d, 1H), 7.76 (d, 1H), 7.37 (s, 1H), 4.10 (m, 1H), 3.69 (s, 3H), 3.57 (s, 2H), 3.24 (t, 2H), 2.78 (t, 2H), 1.32-1.23 (m, 2H), 1.09-1.03 (m, 2H).

### EXAMPLE 54

### 7-[5-(azidomethyl)-4,5,6,7-tetrahydro-1-benzothien-2-yl]-1-cyclopropyl-8-methoxy-4-oxo-1,4-dihydro-3-quinolinecarboxlic acid

A solution of 70A (0.040 g, 0.084 mmol) and aqueous sodium hydroxide (1 mL of a 1 M solution) in ethanol (1 mL) was heated to 40° C. for 16 h. The mixture was cooled to room temperature and acidified with 6 N HCl (0.2 mL) then extracted with ethyl acetate, dried (MgSO₄), and concentrated. The resulting acid was was purified by chromatography on silica gel eluting with 70% acetone in hexane with 0.5% acetic acid then 85% acetone in hexane with 0.5% acetic acid to provide the desired product (0.015 g, 40% yield). MS (ESI) m/z 449 (M−H)⁻; ¹H NMR (300 MHz, CDCl₃) δ 8.90 (s, 1H), 8.23 (d, 1H), 7.75 (d, 1H), 7.35 (s, 1H), 4.10 (m, 1H), 3.67 (s, 3H), 3.39 (d, 2H), 2.90 (m, 3H), 2.42 (m, 1H), 2.11 (m, 2H), 1.66 (m, 1H), 1.27 (m, 2H), 1.05 (m, 2H).

### EXAMPLE 55

### 1-cyclopropyl-7-((5E/Z)-5-(hydroxyimino)-4,5,6,7-tetrahydro-1-benzothien-2-yl)-8-methoxy-4-oxo-1,4-dihydro-3-quinolinecarboxylic acid

The desired product was prepared by substituting Example 53D for Example 48D in Example 49. MS (ESI) m/z 425 (M+H)⁺; ¹H NMR (300 MHz, CDCl₃) δ 8.92 (s, 1H), 8.22 (d, 1H), 7.79 (d, 1H), 7.43 (s, 1H), 4.16 (m, 1H), 3.78 (s, 1H), 3.69 (s, 3H), 3.53 (s, 1H), 3.37 (m, 2H), 3.00 (m, 2H), 2.70 (m, 1H), 1.31 (m, 2H), 1.08 (m, 1H).

### EXAMPLE 56

### 1-cyclopropyl-8-methoxy-7-((5E/Z)-5-(methoxnimino)-4,5,6,7-tetrahydro-1-benzothien-2-yl)-4-oxo-1,4-dihydro-3-quinolinecarboxylic acid

The desired product was prepared by substituting Example 53D for Example 48D and methoxyamine hydrochloride for hydroxylamine hydrochloride in Example 49. MS (ESI) m/z 439 (M+H)⁺; ¹H NMR (300 MHz, DMSO-d6) δ 8.79 (s, 1H), 8.12 (d, 1H), 7.99 (d, 1H), 7.65 (s, 1H), 4.25 (m, 1H), 4.10 (s, 1H), 3.81 (s, 3H major), 3.79 (s, 3H minor), 3.68 (s, 3H), 3.65 (s, 1H), 2.97 (m, 2H), 2.80 (t, 1H), 2.60 (t, 1H), 1.15 (m, 2H), 1.04 (m, 2H).

### EXAMPLE 57

### 1-cyclopropyl-7-(5-methyl-4,5,6,7-tetrahydrothieno[2,3-c]pyrdin-2-yl)-4-oxo-1,4-dihydro[1,8]naphthyridine-3-carboxylic acid hydrobromide

### EXAMPLE 57A

### N-methylene-1-(3-thienyl)-2-propanamine

The desired product was prepared by substituting 3-(2-aminopropyl)-thiophene (prepared by the method of Anne-Archard, et al. Eur. Pat. Appl. 82-401132) for 2-thiophenethylamine in Example 1-A.

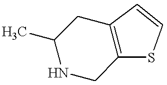

### EXAMPLE 57B

The desired product was prepared by substituting Example 57A for Example 1A in Example 1B.

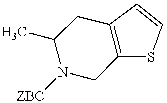

### EXAMPLE 57C

The desired product was prepared by substituting Example 57B for Example 11B in Example 11C.

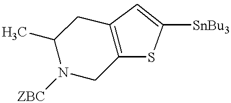

### EXAMPLE 57D

The desired product was prepared by substituting Example 57C for Example 218B in Example 218C.

### EXAMPLE 57E

### ethyl 7-{6-[(benzyloxy)carbonyl]-5-methyl-4,5,6,7-tetrahydrothieno[2,3-c]pyridin-2-yl}-1-cyclopropyl-8-methoxy-4-oxo-1,4-dihydro-3-quinolinecarboxylate

The desired product was prepared by substituting Example 57D and ethyl 7-bromo-1-cyclopropyl-8-methoxy-4-oxo-1,4-dihydro-3-quinolinecarboxylate and a reaction time of 4 hours for Example 1D and 7-bromo-1-cyclopropyl-4-oxo-1,4-dihydro-3-quinolinecarboxylate and a reaction time of 24 hours, respectively in Example 1 E.

### EXAMPLE 57F

### 7-{6-[(benzyloxycarbonyl]-5-methyl-4,5,6,7-tetrahydrothieno2,3-c]-pyridin-2-yl}-1-cyclopropyl-8-methoxy-4-oxo-1,4-dihydro-3-quinolinecarboxulic acid

The desired product was prepared by substituting Example 57E for Example 2A in Example 2B.

### EXAMPLE 57G

### 1-cyclopropyl-7-(5-methyl-4,5,6,7-tetrahydrothieno[2,3-c]pyridin-2-yl)-4-oxo-1,4-dihydro[1,8]naphthyridine-3-carboxylic acid hydrobromide

The desired product was prepared by substituting Example 57F for Example 40B in Example 40C. mp 156-157° C.; MS (APCI) m/z 367 (M+H)⁺; ¹H NMR (300 MHz, DMSO-d₆) δ 9.40 (br s, 2H), 9.10 (br s, 1H), 8.83 (s, 1H), 8.67 (d, 1H), 8.17 (d, 1H), 8.04 (s, 1H), 4.45 (s, 2H), 3.80 (m, 1H), 3.20 (m, 1H), 2.60 (m, 2H), 1.40 (d, 3H), 1.15 (m, 2H), 1.07 (m, 2H).

### EXAMPLE 58

### 7-(5-bromo-4-oxo-4,5,6,7-tetrahydro-1-benzothien-2-yl)-1-cyclopropyl-8-methoxy-4-oxo-1,4-dihydro-3-quinolinecarboxylic acid

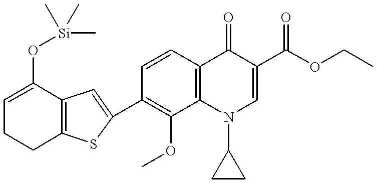

### EXAMPLE 58A

The desired product was prepared by substituting Example 48B and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate in Example 1E.

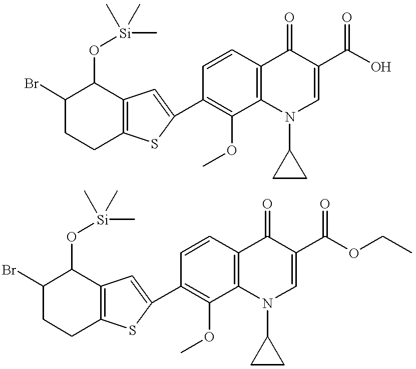

### EXAMPLE 58B

A solution of Example 58A (1.64 g, 3.0 mmol) in dichloromethane (40 mL) was treated with 4-(dimethylamino)pyridinium tribromide (1.45 g, 4.0 mmol) and stirred at room temperature for 3 hours. The reaction mixture was partitioned between water and dichloromethane, dried (Na₂SO₄) and concentrated. The resulting mixture was separated by silica gel chromatography eluting with 1% methanol in dichloromethane to give Example 58B (0.11 g, 8%) and Example 62A . For Example 58B: MS (DCI/NH₃) m/z (M+H)⁺; ¹H NMR (300 MHz, CDCl₃) δ 8.93 (s, 1H), 8.30 (d, 1H), 7.98 (s, 1H), 7.78 (d, 1H), 4.68 (m, 1H), 4.10 (m, 1H), 3.69 (s, 3H), 3.38 (m, 1H), 3.13 (m, 1H), 2.63 (m, 2H), 1.30 (m,2H), (m, 2H).

### EXAMPLE 59

### 1-cyclopropyl-8-methoxy-4-oxo-7-(6-oxo-4,5,6,7-tetrahydro-1-benzothien-2-yl)-1,4-dihydro-3-quinolinecarboxylic acid

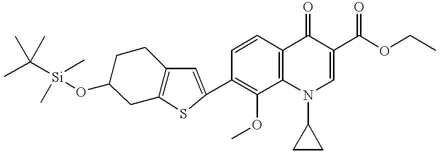

### EXAMPLE 59A

The desired product was prepared by substituting Example 47B and ethyl 7-bromo-1-cyclopropyl-8-methoxy-4-oxo-1,4-dihydro-3-quinolinecarboxylate for Example 1D and ethyl 7-bromo-1-cyclopropyl-8-methoxy-4-oxo-1,4-dihydro-3-quinolinecarboxylate, respectively in Example 1E and was purified by chromatography on silica gel eluting with 20% ethyl acetate in hexanes.

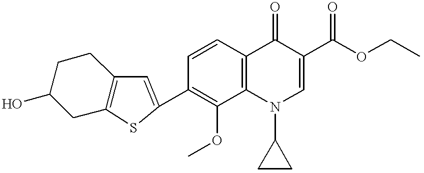

### EXAMPLE 59B

The desired product was prepared by substituting Example 59A for Example 35D in Example 35E and was purified by chromatography on silica gel eluting with 50% ethyl acetate in hexane.

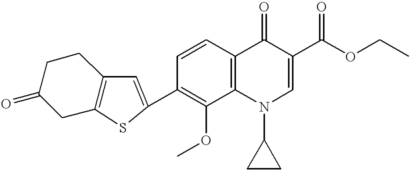

### EXAMPLE 59C

The desired product was prepared by substituting Example 59B for Example 41C in Example 32451 1A and was used without further purification.

### EXAMPLE 59D

### 1-cyclopropyl-8-methoxy-4-oxo-7-(6-oxo-4,5,6,7-tetrahydro-1-benzothien-2-yl)-1,4-dihydro-3-quinolinecarboxylic acid

The desired product was prepared by substituting Example 59C and THF as reaction solvent for Example 59C and EtOH as reaction solvent, respectively in Example 59D. MS (DCI/NH₃) m/z 410 (M+H)⁺; ¹H-NMR (300 MHz, d₆-DMSO) δ 8.79 (s, 1H), 8.12 (d, 1H), 8.03 (d, 1H), 7.70 (s, 1H), 4.25 (m, 1H), 3.72 (s, 2H), 3.70 (s, 3H), 3.03 (dd, 2H), 2.64 (dd, 2H), 1.2-1.05 (m, 4H).

### EXAMPLE 60

### 1-cyclopropyl-7-((6E/Z)-6-(hydroxyimino)-4,5,6,7-tetrahydro-1-benzothien-2-yl)-8-methoxy-4-oxo-1 4-dihydro-3-quinolinecarboxylic acid

The desired product was prepared by substituting Example 59D for Example 59D in Example 49 and the product isolated by partitioning the reaction mixture between ethyl acetate and water, the organic phase dried (Na₂SO₄), and the concentrated residue triturated with hexanes to afford a white solid. MS (DCI/NH₃) m/z 425 (M+H)⁺; ¹H-NMR (300 MHz, d₆-DMSO) δ 14.90 (s, 1H), 8.78 (s, 1H), 8.10 (d, 1H), 7.97 (d, 1H), 7.63 (s, 1H), 4.25 (m, 1H), 3.80 (s, 2H), 3.69 (s, 3H), 3.67-3.5 (m, 2H), 2.78 (m, 2H), 2.56 (m, 1H), 1.3-1.00 (m, 4H).

### EXAMPLE 61

### 1-cyclopropyl-8-methoxy-4-oxo-7-(7-oxo-4,5,6,7-tetrahydro-1-benzothien-2-yl)-1,4-dihydro-3-quinolinecarboxylic acid

### EXAMPLE 61A

A solution of Example 41C (0.05 g, 0.11 mmol) in CH₂Cl₂ (20 mL) was cooled to 0° C., treated with Dess-Martin periodinane (0.06 g, 0.14 mmol), allowed to come to room temperature and stir for 4 hour. The reaction mixture was partitioned between water and dichloromethane, the organic phase washed with sat. Na₂ S₂O₆, 10% K₂CO₃, brine, and dried (Na₂SO₄). Concentration gave a dark oil that was purified by silica gel chromatography eluting with hexane:acetone:methanol (55:45:5) to hexane:acetone:methanol (40:50:10) to provide the desired product (0.028 g, 53% yield) as a tan solid.

### EXAMPLE 61B

### 1-cyclopropyl-8-methoxy-4-oxo-7-(7-oxo-4,5,6,7-terahydro-1-benzothien-2-yl)-1,4-dihydro-3-quinolinecarboxylic acid

The desired product was prepared by substituting Example 6 1A for Example 2A in Example 2B. MS (APCI) m/z 410 (M+H⁺)⁺¹H NMR (300 MHz, DMSO-d₆) δ 8.82 (s, H), 8.17 (d, 1H ), 8. 10 (d,1H), 7.87 (s,1H), 4.27 (m, 1H), 3.71 (s, 3H), 2.93 (t, 2H), 2.60 (t, 2H), 2.14 (m, 2H), 1.17 (s, 2H), 1.06 (s, 2H).

### EXAMPLE 62

### 7-(5-azido-4-oxo-4,5,6,7-tetrahydro-1-benzothien-2-yl)-1-cyclopropyl-8-methoxy-4-oxo-1,4-dihydro-3-quinolinecarboxvlic acid

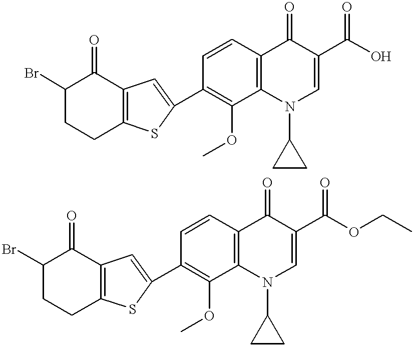

### EXAMPLE 62A

A solution of Example 58A (1.64 g, 3.0 mmol) in dichloromethane (40 mL) was treated with 4-(dimethylamino)pyridinium tribromide (1.45 g, 4.0 mmol) and stirred at room temperature for 3 hours. The reaction mixture was partitioned between water and dichloromethane, dried (Na₂SO₄) and concentrated. The resulting mixture was separated by silica gel chromatography eluting with 1% methanol in dichloromethane to give Example 58B (0.11 g, 8%) and Example 62A . For Example 58: MS (DCI/NH₃) m/z (M+H)⁺; ¹H NMR (300 MHz, CDCl₃) δ 8.93 (s, 1H), 8.30 (d, 1H), 7.98 (s, 1H), 7.78 (d, 1H), 4.68 (m, 1H), 4.10 (m, 1H), 3.69 (s, 3H), 3.38 (m, 1H), 3.13 (m, 1H), 2.63 (m, 2H), 1.30 (m,2H), (m, 2H).

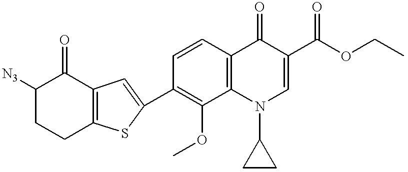

### EXAMPLE 62B

A solution of Example 62A (0.10 g, 0.19 mmol) in DMSO (5 mL) was treated with sodium azide (0.015 g, 0.23 mmol) and stirred at room temperature for 2 hours. The reaction mixture was partitioned between water and dichloromethane, the aqueous layer extracted with dichloromethane, the combined organic layers washed with brine, dried (Na₂SO₄), and concentrated to yield the desired product (0.054 g, 58%).

### EXAMPLE 62C

### 7-(5-azido-4-oxo-4,5,6,7-tetrahydro-1-benzothien-2-yl)-1-cyclopropyl-8-methoxy-4-oxo-1,4-dihydro-3-quinolinecarboxylic acid

The desired product was prepared by substituting Example 62B for Example 48C in Example 48D. MS (DCI/NH₃) m/z 451 (M+H)⁺; ¹H NMR (300 MHz, CDCl₃) δ 8.93 (s, 1H), 8.30 (d, 1H), 7.96 (s, 1H), 7.77 (d, 1H), 4.26 (dd, 1H), 4.10 (m, 1H), 3.68 (s, 3H), 3.22 (m, 2H), 2.31 (m, 2H), 2.48 (m, 2H), 1.29 (m, 2H), 1.06 (m, 2H).

### EXAMPLE 63

### 1-cyclopropyl-8-methoxy-7-((7E/Z)-7-(methoxyimino)-4,5,6,7-tetrahydro-1-benzothien-2-yl)-4-oxo-1,4-dihydro-3-quinolinecarboxylic acid

The desired product was prepared by substituting Example 61B for Example 48D in Example 49. MS (ESI) m/z 408 (M-OCH₃+H)⁻¹H NMR (300 MHz, DMSO-d₆) δ 8.73 (s, 1H), 8.12 (d, 1H), 7.96 (d, 1H), 7.82 (s, 1H), 4.17 (m, 1H), 4.02 (s, 3H), 3.68 (s, 3H), 2.92 (t, 2H), 2.59 (t, 2H), 2.12 (m, 2H), 1.14 (d, 2H), 0.95 (s, 2H).

### EXAMPLE 64

### 1-cyclopropyl-8-methoxy-7-((4E/Z)-4-(methoxyimino)-4,5,6,7-tetrahydro-1-benzothien-2-yl)-4-oxo-1,4-dihydro-3-quinolinecarboxylic acid

A solution of Example 50 (0.120 g, 0.3 mmol) was suspended in 4 mL of MeOH and treated with NaOAc (0.125 g, 1.5 mmol) and methoxylamine hydrochloride (0.218 g, 1.6 mmol). The reaction mixture was heated at 90° C. for 18 hours. The solvent was removed and 20 mL of 10% NH₄Cl was added. The product was isolated by suction filtration and was dried under vacuum giving the desired product as a white solid (0.100 g, 76%). mp 232-233° C.; MS (APCI) m/z 439 (M+H)⁺; ¹H NMR (300 MHz, DMSO-d6) δ 8.80 (s, 0.85H), 8.41 (s, 0.15H), 8.13 (d, 0.15H), 8.11 (d, 0.85H), 8.02 (d, 0.85H), 7.98 (d, 0.15H), 7.89 (s, 1H), 4.27 (m, 1H), 3.92 (s, 0.45H), 3.90 (s, 2.55H), 3.70 (s, 3H), 3.30 (s, 1H), 3.01 (dd, 0.3H), 2.91 (dd, 1.70H), 2.81 (dd, 0.3H), 2.68 (dd, 1.70H), 2.01 (m, 0.3H), 1.97 (m, 1.7H), 1.18 (m, 2H), 1.05 (m, 2H).

### EXAMPLE 65

### 1-cyclopropyl-7-((4E/Z)-4-(ethoxyimino)-4,5,6,7-tetrahydro-1-benzothien-2-yl)-8-methoxy-4-oxo-1,4-dihydro-3-quinolinecarboxylic acid

The desired product was prepared by substituting O- ethylhydroxylamine hydrochloride for hydroxylamine hydrochloride in Example 49. mp 208-210° C.; MS (APCI) m/z 453 (M+H)⁺; ¹H NMR (300MHz, DMSO-d₆) δ 8.82 (s, 1H), 8.13 (d, 1H), 8.05 (d, 1H), 7.88 (s, 1H), 4.27 (m, 1H), 4.15 (q, 2H), 3.69 (s, 3H), 3.30(s, 1H), 2.90 (dd, 2H), 2.67 (dd, 2H), 1.92 (m, 2H), 1.28 (t, 3H), 1.15 (m, 2H), 1.05 (m, 2H).

### EXAMPLE 66

### 1-cyclopropyl-8-methoxy-7-((6E/Z)-6-(methoxyimino)-4,5,6,7-tetrahydro-1-benzothien-2-yl)-4-oxo-1,4-dihydro-3-quinolinecarboxylic acid

The desired product was prepared by substituting Example 59D and O-methylhydroxylamine for Example 59D and hydroxylamine hydorchloride, respectively in Example 49 and the product isolated by partitioning the reaction mixture between ethyl acetate and water, the organic phase dried (Na₂SO₄), and the concentrated residue triturated with hexanes to afford a white solid. MS (DCI/NH₃) m/z 439 (M+H)⁺; ¹H-NMR (300 MHz, d₆-DMSO) δ 8.79 (s, 1H), 8.10 (d, 1H), 8.00 (d, 1H), 7.63 (s, 1H), 4.25 (m, 2H), 3.83 (s, 3H), 3.68 (s, 3H), 2.80-2.68 (m, 1H), 2.60-2.45 (m, 2H), 1.30 (m, 2H), 1.15 (m, 2H), 1.05 (m, 2H).

### EXAMPLE 67

### 1-cyclopropyl-7-(6,7-dihydro-1-benzothien-2-yl)-8-methoxy-4-oxo-1,4-dihydro-3-quinolinecarboxylic acid

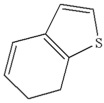

### EXAMPLE 67A

A solution of p-toluenesulfonic anhydride (13.7 g, 42.0 mmol) in CH₂Cl₂ (70 mL) was added dropwise to a solution of Example 198A (5.33g, 35.0 mmol) and 4-dimethylaminopyridine (5.50g, 45.5 mmol) in CH₂Cl₂ at 0° C. The reaction mixture was slowly warmed to room temperature overnight, diluted with ethyl acetate, washed with saturated aqueous sodium bicarbonate, saturated aqueous ammonium chloride, dried (Na₂SO₄) and concentrated. The resulting oil was purified by chromatography on silica gel eluting with 10% ethyl acetate in hexane to provide the desired product (3.25 g, 68%) as a pale yellow oil.

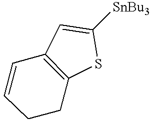

### EXAMPLE 67B

The desired product was prepared by substituting Example 67A for Example 1C in Example 1D.

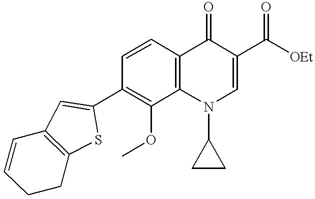

### EXAMPLE 67C

The desired product was prepared by substituting Example 67B and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E.

### EXAMPLE 67D

### 1-cyclopropyl-7-(6,7-dihydro-1-benzothien-2-yl)-8-methoxy-4-oxo-1,4-dihydro-3-quinolinecarboxylic acid

The desired product was prepared by substituting Example 67C for Example 2A in Example 2B. MS (DCI/NH₃) m/z 394 (M+l)⁺; ¹H NMR (300 MHz, DMSO-d₆) δ 8.79 (s, 1H), 8.11 (d, 1H), 8.00 (d, 1H), 7.68 (s, 1H), 6.54 (dt, 1H), 5.92 (dt, 1H), 4.25 (m, 1H), 3.69 (s, 3H), 2.92 (t, 2H), 2.46 (m, 2H), 1.17-1.03 (m, 4H).

### EXAMPLE 68

### 1-cyclopropyl-8-methoxy-7-((4E/Z)-4-(4-morpholinvlimino)-4,5,6,7-tetrahydro-1-benzothien-2-yl)-4-oxo-1,4-dihydro-3-quinolinecarboxylic acid

The desired product was prepared by substituting N-aminomorpholine and a reaction time of 48 hours for l-aminopyrrolidine hydrochloride and a reaction time of 24 hours in Example 74. mp 118-120° C.; MS (APCI) m/z 494 (M+H)⁺; ¹H NMR (300MHz, CDCl₃) δ 14.70 (br s, 1H), 8.90 (s, 1H), 8.25 (d, 1H), 7.87 (d, 1H), 7.38 (s, 1H), 4.10 (m, 1H), 3.85 (m, 4H), 3.67 (s, 3H), 3.10 (m, 2H), 2.94 (m, 2H), 2.82 (m, 4H), 2.05 (m, 2H), 1.28 (m, 2H)m 1.04 (m, 2H).

### EXAMPLE 69

### 1-cyclopropyl-7-(4,5-dihydroxy-4,5,6,7-tetrahydro-1-benzothien-2-yl)-8-methoxy-4-oxo-1,4-dihydro-3-quinolinecarboxylic acid

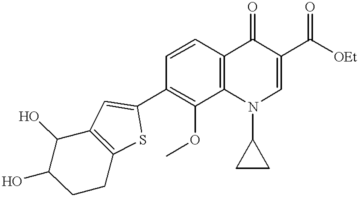

### EXAMPLE 69A

A solution of Example 67C (0.130g, 0.30mmol) in 1:1:1 t-butanol:acetone:H₂O was treated with 4-methyl morpholine N-oxide (0.060g, 0.51 mmol) and osmium tetraoxide (0.005g, 0.02 mmol). The mixture was stirred at 0C for 7 h. Sodium sulfite (0.400g) was added, the reaction was warmed to 25° C., stirred for 1 h, and partitioned between CH₂Cl₂ and water. The layers were separated, and the aqueous layer was extracted 3 times with CH₂Cl₂. The combined organic layers were dried (Na₂SO₄),and concentrated The residue was purified by chromatography on silica gel eluting with CH₂Cl₂ then 5% MeOH in CH₂Cl₂ to provide the desired product as an off-white solid (0.085 g, 62%).

### EXAMPLE 69B

### 1-cyclopropyl-7-(4,5-dihydroxy-4,5,6,7-tetrahydro-1-benzothien-2-yl)-8-methoxy-4-oxo-1,4-dihydro-3-quinolinecarboxylic acid

The desired product was prepared by substituting Example 69A for Example 2A in Example 2B. MS (DCI/NH₃) m/z 428 (M+1)⁺; ¹H NMR (300 MHz, DMSO-d₆) δ 8.79 (s, 1H), 8.12 (d, 1H), 7.99 (d, 1H), 7.70 (s, 1H), 4.92, (m, 1H), 4.56 (m, 1H), 4.52 (m, 1H), 4.25 (m, 1H), 3.72 (m, 1H), 3.68 (s, 3H), 2.95-2.70 (m, 2H), 2.15-1.70 (m, 2H), 1.20-1.03 (m, 4H).

### EXAMPLE 70

### 7-(5-(aminomethyl)-4,5,6,7-tetrahydro-1-benzothien-2-yl)-1-cyclopropyl-8-methoxy-4-oxo-1,4-dihydro-3-quinolinecarboxylic acid hydrochloride

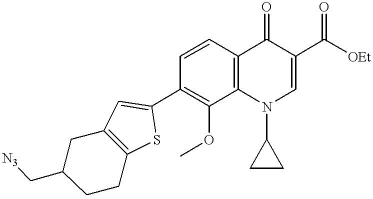

### EXAMPLE 70A

The desired product was prepared by substituting Example 43D for Example 37A and DMF at 65° C. for toluene:dichloromethane at room temperature in Example 37B.

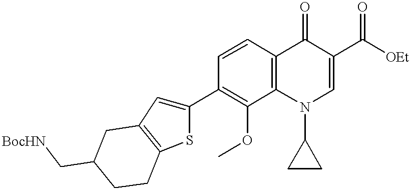

### EXAMPLE 70B

The desired product was prepared by substituting Example 70A for Example 201C in Example 201D.

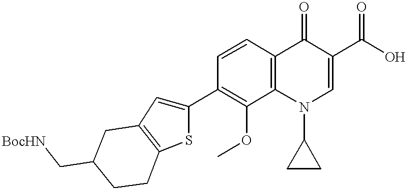

### EXAMPLE 70C

A solution of Example 70B (0.660 g, 1.2 mmol) in ethanol (3 mL) was treated with aqueous sodium hydroxide (3 mL of a 1 M solution) for 17 h at room temperature. The mixture was acidified with acetic acid (8 mL) then concentrated. The resulting acid was purified by chromatography on silica gel eluting with 30% acetone in hexane with 0.5% acetic acid then 40% acetone in hexane with 0.5% acetic acid to provide the desired product (0.590 g, 94%) as an off white solid.

### EXAMPLE 70D

### 7-(5-(aminomethyl)-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 70C for Example 40B in Example 40C. MS (ESI) m/z 425 (M+H)⁺; ¹H NMR (300 MHz, DMSO-d₆) δ 8.80 (s, 1H), 8.11 (d, 1H), 8.01 (d, 1H), 7.94 (br s, 2H), 7.58 (s, 1H), 4.25 (m, 1H), 3.68 (s, 3H), 2.93-2.74 (m, 5H), 2.38 (m, 1H), 2.07 (m, 2H), 1.52 (m, 1H), 1.15 (m, 2H), 1.03 (m, 2H).

### EXAMPLE 71

### 7-(6-amino-4,5,6,7-tetrahydro-1-benzothien-2-yl)-1-cyclopropyl-8-methoxy-4-oxo-1,4-dihydro-3-quinolinecarboxylic acid, trifluoroactic acid salt

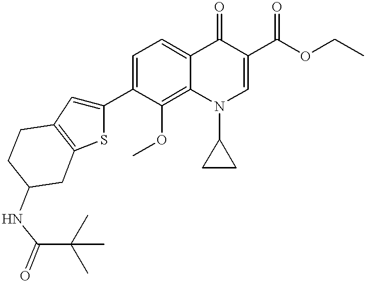

### EXAMPLE 71A

A solution of Example 59C (0.061 g, 0.139 mmol) in MeOH (6 ml) was treated with 3 A molecular sieves, ammonium acetate (0.107 g, 1.39 mmol) and sodium cyanoborohydride (0.009 g, 0.139mmol) and was stirred at room temperature for 2 hours. The reaction mixture was treated with 2 drops AcOH followed by di-tert-butyldicarbonate (0.303 g, 1.39 mmol), stirred for an additional 2 hours and partitioned between saturated aqueous NH4Cl and ethyl acetate. The aqueous layer was extracted with ethyl acetate, the combined organic layers washed with water, brine, dried (MgSO4) and concentrated. The resulting residue was purified by column chromatography on silica gel eluting with 97:3 / CH2Cl2:MeOH to give the desired product (0.069 g, 93%).

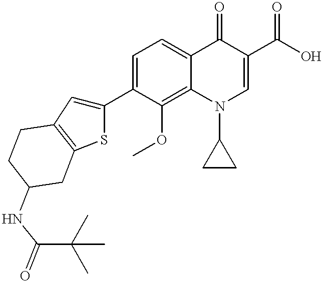

### EXAMPLE 71B

The desired product was prepared by substituting Example 71A for Example 2A in Example 2B.

### EXAMPLE 71 C

### 7-(6-amino-4,5,6,7-tetrahydro-1-benzothien-2-yl)-1-cyclopropyl-8-methoxy-4-oxo-1,4-dihydro-3-quinolinecarboxylic acid, trifluoroactic acid salt

The desired product was prepared by substituting Example 71B for Example 40B in Example 40C. MS (APCI) m/z 411 (M+H)⁺; ¹H NMR (300 MHz, DMSO-d₆) δ 8.80 (s, 1H), 8.12 (d, 1H), 8.05 (br s, 3H), 8.00 (d, 1H), 7.62 (s, 1H), 4.26 (m, 1H), 3.68 (s, 3H), 3.60 (m, 1H), 3.21 (m, 2H), 2.89-2.65 (m, 2H), 2.12 (m, 1H), 1.83 (m, 1H), 1.15 (m, 2H), 1.03 (m, 2H).

### EXAMPLE 72

### 7-((4E/Z)-4-(tert-butoxyimino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting O-t-butylhydroxylamine hydrochloride for hydroxylamine in Example 49. mp 238-240° C.; MS (APCI) m/z 481 (M+H)⁺; ¹H NMR (300MHz, CDCl₃) δ 14.73 (br s, 1H), 8.91 (s, 1H), 8.26 (d, 1H), 7.90 (s, 1H), 7.87 (d, 1H), 4.10 (m, 1H), 3.69 (s, 3H), 2.90 (dd, 2H), 2.74 (dd, 2H), 2.00 (dd, 2H), 1.35 (s, 9H), 1.28 (m, 2H), 1.05 (m, 2H).

### EXAMPLE 73

### 7-((4E/Z)-4-((benzyloxyimino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting O-benzylhydroxylamine hydrochloride and a reaction time of 9 hours for hydroxylamine hydrochloride and a reaction time of 5 hours, respectively in Example 49. mp 105-107° C.; MS (APCI) m/z 515 (M+H)⁺; ¹H NMR (300MHz, DMSO-d₆) δ 14.70 (s, 0.7H), 14.60 (s, 0.3H), 8.90 (s, 0.7H), 8.88 (s, 0.3h), 8.25 (d, 0.7H), 8.20 (d, 0.3H), 7.94 (s, 0.3H), 7.88 (s. 0.7H), 7.82 (d, 0.7H), 7.38 (m, 5H), 7.28 (d, 0.3H), 5.22 (s 1.4H), 5.17 (s, 0.6H), 4.10 (m, 1H), 3.67 (s, 2.1H), 3.57 (s, 0.9H), 3.11 (dd, 0.6H), 3.00 (dd, 0.6H), 2.90 (dd, 1.4H), 2.80 (dd, 1.4H), 2.10(dd, 0.6H), 2.00 (dd, 1.4H), 1.30 (m, 2H), 1.05 (m, 2H).

### EXAMPLE 74

### 1-cyclopropyl-8-methoxy-4-oxo-7-((4E/Z)-4-(1-pyrrolidinylimino)-4,5,6,7-tetrahydro-1-benzothien-2-yl)-1,4-dihydro-3-quinolinecarboxylic acid

A solution of Example 48C (0.180 g, 0.41 numol) in absolute EtOH (6 mL) under a positive N₂ atmosphere was treated with 1 -aminopyrrolidine hydrochloride (0.151 g, 1.23 mmol) and Et₃N (210 μL, 1.50 mmol). The reaction mixture was heated at 75° C. for 24 hours the solvent removed and the residue dissolved in CH₂Cl₂, washed with water, dried (Na₂SO₄), and concentrated.

A solution of the resulting solid (0.180 g, 0.35 mmol) in THF (5 mL) was treated with LiOH-H₂O ( 0.147 g, 3.5 mmol) in water (7.0 mL) at 25° C. for 4 hours. The solution wasd adjusted to pH 7 with 10% NH₄Cl and 1M H₃PO₄ extracted with CH₂Cl₂, the combined organic phases dried (Na₂SO₄) and concentrated to give the desired product as a solid (0.130 g, 77%). mp 90-91° C.; MS (APCI) m/z 478 (M+H)⁺; ¹H NMR (300MHz, CDCl₃) δ 14.75 (br s, 1H), 8.89 (s, 1H), 8.22 (d, 1H), 7.93 (s, 1H), 7.88 (d, 1H), 4.12 (m, 1H), 3.68 (s, 3H), 3.10 (m, 4H), 2.90 (dd, 2H), 2.72 (dd, 2H), 2.08 (m, 2H), 1.88 (m, 4H), 1.35 (m, 2H), 1.07 (m, 2H).

### EXAMPLE 75

### 7-(4-amino-4,5,6,7-tetrahydro-1-benzothien-3-yl)-1-cyclopropyl-8-methoxy-4-oxo-1,4-dihydro-3-quinolinecarboxylic acid

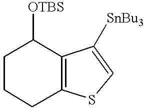

### EXAMPLE 75A

A solution of Example 260C (2.98 g, 8.6 mmol) in diethyl ether (18 mL) at −65° C. was treated dropwise with n-butyllithium (3.96 mL of a 2.5 M solution in hexane, 9.89 mmol) and was stirred at −65° C. for 30 min. The mixture was treated dropwise with chlorotributylstannane (2.33 mL, 8.6 mmol) then allowed to warm to room temperature and partitioned between ethyl acetate and water. The organic layer was washed with brine, dried (Na₂SO₄) and concentrated to provide the desired product as an orange oil (4.78 g, quantitative yield).

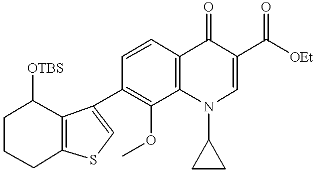

### EXAMPLE 75B

The desired product was prepared by substituting Example 75A, and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinoline-3-carboxylate respectively in Example 1E and the resulting ethyl ester was purified by chromatography on silica gel eluting with 10% acetone in hexane.

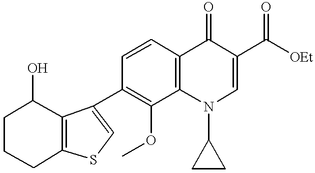

### EXAMPLE 75C

The desired product was prepared by substituting Example 75B for Example 35D in Example 35E and the resulting alcohol was purified by chromatography on silica gel eluting with 30% acetone in hexane.

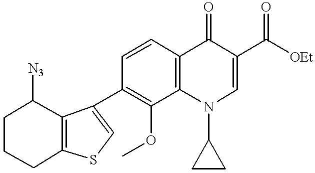

### EXAMPLE 75D

The desired product was prepared by substituting Example 75C for Example 37A in Example 37B and the resulting azide was purified by chromatography on silica gel eluting with 20% acetone in hexane.

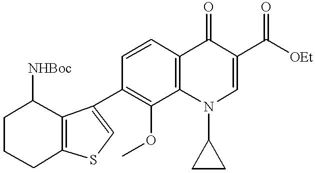

### EXAMPLE 75E

The desired product was prepared by substituting Example 75D for Example 201C in Example 201D and the resulting alcohol was purified by chromatography on silica gel eluting with 20% acetone in hexane.

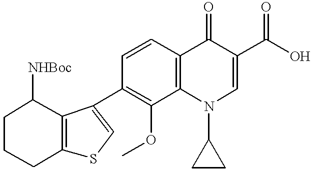

### EXAMPLE 75F

The desired product was prepared by substituting Example 75E and a reaction time of 4 h for Example 2A and a reaction time of 2 h in Example 2B. The resulting acid was purified by chromatography on silica gel eluting with 30% acetone in hexane.

### EXAMPLE 75G

### 7-(4-amino-4,5,6,7-tetrahydro-1-benzothien-3-yl)-1-cyclopropyl-8-methoxy-4-oxo-1,4-dihydro-3-quinolinecarboxylic acid

The desired product was prepared by substituting Example 75F for Example 40B in Example 40C. MS (ESI) m/z 411 (M+H)⁺; ¹H NMR (300 MHZ, DMSO-d₆) δ 8.84 (s, 1H), 8.20 (d, 1H), 7.84 (br s, 2H), 7.61 (s, 1H), 7.58 (d, 1H), 4.56 (s, 1H), 4.24 (m, 1H), 3.41 (s, 3H), 2.94 (m, 2H), 2.16 (m, 1H), 1.96 (m, 3H), 1.26 (m, 2H), 1.10 (m, 2H).

### EXAMPLE 76

### 7-(5 -amino-4,5,6,7-tetrahydro-1-benzothien-2-yl)-1-cyclopropyl-8-methoxy-4-oxo-1,4-dihydro-3-quinolinecarboxylic acid hydrochloride

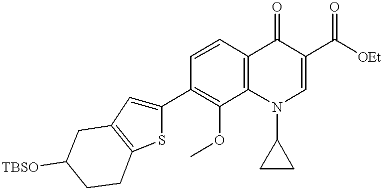

### EXAMPLE 76A

The desired product was prepared by substituting Example 42B, ethyl 7-bromo-1-cyclopropyl-8-methoxy-4-oxo-1,4-dihydro-3-quinolinecarboxylate and a reaction time of 4 h for Example 1D, ethyl 7-bromo-1-cyclopropyl-4-oxo-1,4-dihydro-3-quinolinecarboxylate and a reaction time of 24 h in Example 1E.

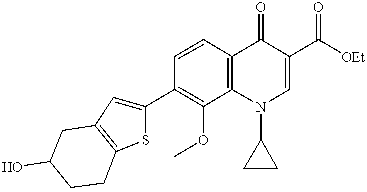

### EXAMPLE 76B

The desired product was prepared by substituting Example 76A for Example 35D in Example 35 E and the resulting alcohol was purified by chromatography on silica gel eluting with CH₂Cl₂ then 3% MeOH in CH₂Cl₂.

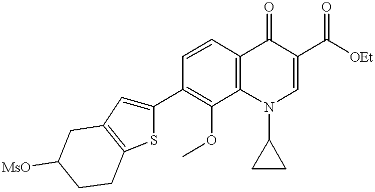

### EXAMPLE 76C

The desired product was prepared by substituting Example 76B for Example 45G in Example 249A.

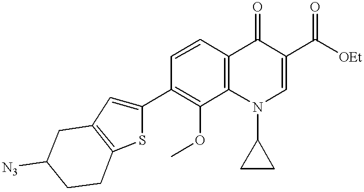

### EXAMPLE 76D

The desired product was prepared by substituting Example 76C for Example 249A in Example 249B.

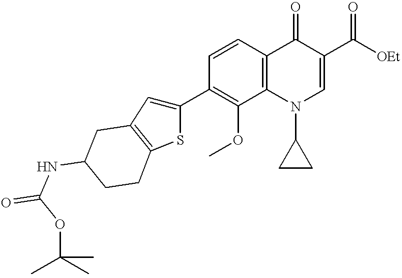

### EXAMPLE 76E

The desired product was prepared by substituting Example 76D for Example 201A in Example 201B.

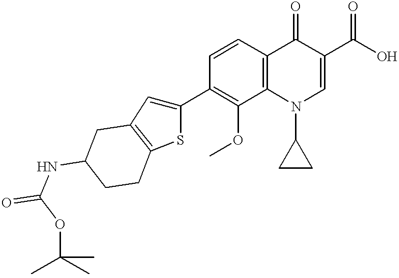

### EXAMPLE 76F

The desired product was prepared by substituting Example 76E for Example 2A in Example 2B.

### EXAMPLE 76G

### 7-(5-amino-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 76F for Example 40B in Example 40C. MS (DCI/NH3) m/z 411 (M+1)⁺; ¹H NMR (300 MHz, MeOH-d₄) δ 8.95 (s, 1H), 8.20 (d, 1H), 7.90 (d, 1H), 7.51 (s, 1H), 4.29 (m, 1H), 3.70 (m, 4H), 3.20 (m, 1H), 3.05 (m, 2H), 2.25 (m, 1H), 2.32 (m, 1H) 2.01 (m, 1H), 1.30-1.23 (m, 2H), 1.09-1.04 (m, 2H).

### EXAMPLE 77

### 1-cyclopropyl-8-methoxy-7-[4-(methylamino)-4,5,6,7-tetrahydro-1-benzothien-2-yl]-4-oxo-1,4-dihydro-3-quinolinecarboxylic acid hydrochloride

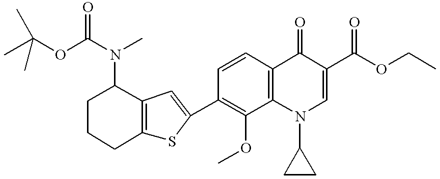

### EXAMPLE 77A

A solution of Example 40A (0.44 g, 0.82 mmol) in tetrahydrofaran (5 mL) was added dropwise to a solution of 1.0M sodium bis(tirmethylsilyl)amide (1.48 mL, 1.47 mmol) in tetrahydrofuran (20 mL) at −70° C. This mixture was stirred for 2 hours then treated with iodomethane (0.20 mL, 3.28 mmol) and stirred at 10° C. for 16 hours. The reaction mixture was partitioned between 10% NH₄Cl and dichloromethane, dried (Na₂SO₄), concentrated, and purified by silica gel column eluting with 10% hexane in ethyl acetate to yield the desired product (0.28 g, 48%).

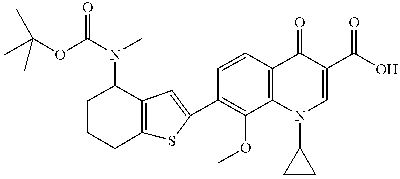

### EXAMPLE 77B

The desired product was prepared by substituting Example 77A for Example 2A in Example 2B.

### EXAMPLE 77C

### 1-cyclopropyl-8-methoxy-7-[4-(methylamino)-4,5,6,7-tetrahydro-1-benzothien-2-yl]-4-oxo-1,4-dihydro-3-quinolinecarboxylic acid hydrochloride

The desired product was prepared by substituting Example 77B for Example 40B in Example 40C. MS (DCI/NH₃) m/z 425 (M+H)⁺; ¹H NMR (300 MHz, CD₃OD) δ 8.97 (s, 111), 8.23 (d, 111), 7.93 (d, 1H), 7.78 (s, 1H), 4.47 (m, 1H), 4.29 (m, 1H), 3.72 (s, 3H), 2.98 (m, 2H), 2.82 (s, 3H), 2.01-2.29 (m, 4H), 1.28 (m, 2H), 1.08 (m, 2H).

### EXAMPLE 78

### 1-cyclopropyl-7-((5E/Z)-5-(ethoxyimino)-4,5,6,7-tetrahydro-1-benzothien-2-yl)-8-methoxy-4-oxo-1,4-dihydro-3-quinolinecarboxylic acid

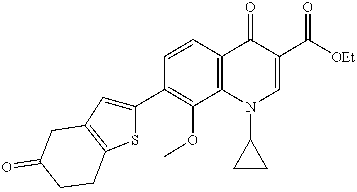

### EXAMPLE 78A

The desired product was prepared by substituting Example 76B for Example 41C in Example 61A.

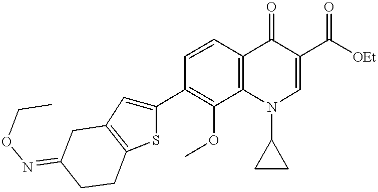

### EXAMPLE 78B

The desired product was prepared by substituting Example 78A for Example 48D, ethoxyamine hydrochloride for hydroxylamine hydrochloride, and a reaction time of 17 h at room temperature for 24 h at 75° C. in Example 61A.

### EXAMPLE 78C

### 1-cyclopropyl-7-((5E/Z)-5-(ethoxyimino)-4,5,6,7-tetrahydro-1-benzothien-2-yl)-8-methoxy-4-oxo-1,4-dihydro-3-quinolinecarboxylic acid

The desired product was prepared by substituting Example 78B for Example 2A in Example 2B. MS (ESI) m/z 453 (M+H)⁺; ¹H NMR (300 MHz, DMSO-d₆) δ 8.79 (s, 1H), 8.11 (d, 1H), 7.98 (d, 1H), 7.52 (s, 1H), 4.25 (m, 1H), 4.07 (q, 2H), 3.69 (s, 3H), 3.63 (m, 2H), 2.96 (m, 2H), 2.81 (m, 1H), 2.61 (m, 1H), 1.22 (t, 3H), 1.04 (m, 2H), 0.86 (m, 2H).

### EXAMPLE 79

### 7-((5E/Z)-5-((benzyloxy)imino)-4,5,6,7-tetrahydro-1-benzothien-2-yl)-1-cyclopropyl-8-methoxy-4-oxo-1,4-dihydro-3-quinolinecarboxylic acid

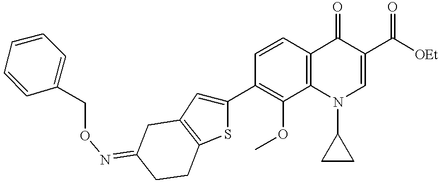

### EXAMPLE 79A

The desired product was prepared by substituting Example 78A for Example 48D, O-benzylhydroxylamine hydrochloride for hydroxylamine hydrochloride, and a reaction time of 17 h at room temperature for 24 h at 75° C. in Example 61A.

### EXAMPLE 79B

### 7-((5E/Z)-5-((benzyloxy)imino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting Example 79A for Example 2A in Example 2B. MS (ESI) m/z 515 (M+H)⁺; ¹H NMR (300 MHz, DMSO-d₆) δ8.79 (s, 1H), 8.11 (d, 1H), 7.97 (d, 1H), 7.66 (s, 1H), 7.37 (m, 5H), 5.10 (s, 2H), 4.26 (m, 1H), 3.78 (m, 2H), 3.68 (s, 3H), 3.46 (m, 1H), 2.98 (m, 2H), 2.66 (m, 1H), 1.16 (m, 2H), 1.04 (M, 2H).

### EXAMPLE 80

### 7-((4E/Z)-4-((aminocarbonyl)hydrazono)-4,5,6,7-tetrahydro-1-benzothien-2-yl)-1-cylopropyl-8-methoxy-4-oxo-1,4-dihydro-3-quinolinecarboxylic acid

The desired product was prepared by substituting semicarbazide hydrochloride and a reaction time of 48 hours for 1-aminopyrrolidine hydrochloride and a reaction time of 24 hours in Example 74. mp 243-244° C.; MS (APCI) m/z 467 (M+H)⁺; ¹H NMR (300 MHz, DMSO-d₆) δ 9.28 (br s, 1H), 8.80 (s, 1H), 8.40 (s, 1H), 8.23 (d, 1H), 8.13 (d, 1H), 7.30 (br s, 2H), 6.60 (br s, 1H), 4.26 (m, 1H), 3.71 (s, 3H), 2.89 (dd, 2H), 2.57 (dd, 2H), 1.95 (m, 2H), 1.20 (m, 2H), 1.03 (m, 2H).

### EXAMPLE 83

### 1-cyclopropyl-7-(4-(dimethylamino)-4,5,6,7-tetrahydro-1-benzothien-2-yl)-8-methoxy-4-oxo-1,4-dihydro-3-quinolinecarboxylic acid hydrochloride

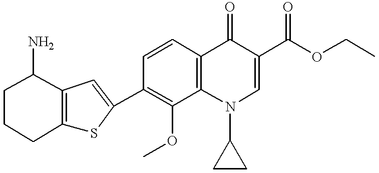

### EXAMPLE 83A

The desired product was prepared by substituting Example 40A for Example 40B in Example 40C and the crude reaction mixture partitioned between water and dichloromethane. The aqueous layer was adjusted to pH 9 with sodium hydroxide, extracted with dichloromethane, dried (Na₂SO₄), filtered, and concentrated to give a yellow solid that was used without further purification.

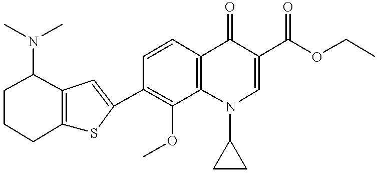

### EXAMPLE 83B

A solution of Example 83A (0.24 g, 0.55 mmol) in tetrahydrofuran (2 mL) was added to a cooled solution (−70° C.) of 1.0M sodium bis(tirmethylsilyl)amide (1.37 mL, 1.38 mmol) and stirred for 2 hours. lodomethane (0.14 g, 2.20 mmol) was added and the mixture was warmed to room temperature and stired for 3 hours. The reaction mixture was partitioned between 10% NH₄Cl and dichloromethane, dried (Na₂SO₄), concentrated, and purified by silica gel column eluting with a gradient of 1% to 6% methanol in dichloromethane to yield (0.055 g, 22%) of the desired product.

### EXAMPLE 83C

### 1-cyclopropyl-7-(4-(dimethylamino)-4,5,6,7-tetrahydro-1-benzothien-2-yl)-8-methoxy-4-oxo-1,4-dihydro-3-quinolinecarboxylic acid hydrochloride

The desired product was prepared by substituting Example 83B for Example 2A in Example 2B followed by treatment with 4M HCl in dioxane and filtration provide the solid product. MS (DCI/NH₃) m/z 439 (M+H)⁺; ¹H NMR (300 MHz, CD₃OD) δ 8.97 (s, 1H), 8.24 (d, 1H), 7.93 (d, 1H), 7.81 (, 1H), 4.74 (m, 1H), 4.29 (m, 1H), 3.72 (s, 3H), 2.97 (m, 2H), 2.92 (d, 3H), 2.17 (m, 2H), 1.99 (m, 2H), 1.28 (m, 2H), 1.08 (m, 2H).

### EXAMPLE 84

### 7-((4E/Z)-4-[(aminocarbothioyl)hydrazono)-4,5,6,7-tetrahydro-1-benzothien-2-yl)-1-cyclopropyl-8-methoxy-4-oxo-1,4-dihydro-3-quinolinecarboxylic acid

The desired product was prepared by substituting thiosemicarbazide and a for 1-aminopyrrolidine hydrochloride and a reaction time of 24 hours in Example 331536 and was purified by silica gel chromatography eluting with 3.5 % MeOH in dichloromethane. mp 235-236° C.; MS (APCI) m/z 481 (M-H)⁻483 (M+H)⁺; ¹H NMR (300 MHz, DMSO-d₆) δ 8.70 (s, 1H), 8.50 (s, 1H), 8.32 (d, 1H), 8.12 (d, 1H), 4.28 (m, 1H), 3.72 (s, 3H), 3.30 (m, 4H), 2.91 (dd, 2H), 2.69 (dd, 2H), 1.96 (dd, 2H), 1.20 (m, 2H), 1.07 (m, 2H).

### EXAMPLE 85

### 1-cyclopropyl-8-methoxy-7-((4E/Z)-4-((methylamino)carbothioyl)hydrazono)-4,5,6,7-tetrahydro-1-benzothien-2-yl)-4-oxo-1,4-dihydro-3-quinolinecarboxylic acid

The desired product was prepared by substituting 4-methyl-3-thiosemicarbazide, a reaction time of 48 hours and a reaction temperature of 100° C. for 1-aminopyrrolidine hydrochloride, a reaction time of 24 hours and a reaction temperature of 75° C. in Example 331536. mp 272-273° C.; MS (APCI) m/z 495 (M-H)⁻497 (M+H)⁺; ¹H NMR (300 MHz, DMSO-d₆) δ 8,80 (s, 1H), 8.53 (d, 1H), 8.40 (s, 1H), 8.16 (d, 1H), 4.25 (m, 1H), 3.72 (s, 3H), 3.30 (m, 3H), 3.08 (d, 3H), 2.90 (dd, 2H), 2.68 (dd, 2H), 1.97 (dd, 2H), 1.20 (m, 2H), 1.08 (m, 2H).

### EXAMPLE 86

### 1-cyclopropyl-8-methoxy-7-(5-methylene-4-oxo-4,5,6,7-tetrahydro-1-benzothien-2-yl)-4-oxo-1,4-dihydro-3-quinolinecarboxylic acid

### EXAMPLE 86A

### ethyl 1-cyclopropyl-8-methoxy-7-(5-methylene-4-oxo-4,5,6,7-tetrahydro-1-benzothien-2-yl)-4-oxo- 1,4-dihydro-3-quinolinecarboxylate

A mechanically stirred solution of dimethylamine hydrochloride (3.70 g, 0.045 mol), paraformaldehyde (1.30 g, 0.045 mol), and 10 mL conc. HCI in (4:1) DME:dichloroethane. (125 mL) was heat to 90° C. for 10 minutes, treated dropwise with a solution of Example 50A (1.0 g, 0.0023 mol) in DME (35 mL) and heated to 110-125° C. for 4.5 hour. The reaction mixture was cooled, filtered, and the filtrate concentrated. The residue was dissolved in CH₂Cl₂, washed with sat. NaHCO₃, brine, and dried (Na₂SO₄). The concentrated residue was triturated in ethyl ether and filtered to give the desired product as a yellow solid (0.72 g, 70%).

### EXAMPLE 86B

### 1-cyclopropyl-8-methoxy-7-(5-methylene-4-oxo-4,5,6,7-tetrahydro-1-benzothien-2-yl)-4-oxo-1,4-dihydro-3-quinolinecarboxylic acid

The desired product was prepared by substituting Example 86A for Example 2A in Example 2B. MS (APCI) m/z 421 (M+H)⁺¹H NMR (300 MHz, CDCl₃) δ 14.6 (s, 1H), 8.92 (s, 1H), 8.28 (d, 1H), 8.02 (s, 1H), 7.80 (d, 1H), 6.23 (s, 1H), 5.52 (s, 1H), 4.10 (m,1H), 3.68 (s, 3H), 3.17 (t, 2H), 3.02 (t, 2H), 1.30 (d, 2H), 1.06 (s, 2H).

### EXAMPLE 88

### 1-cyclopropyl-8-methoxy-7-(4-((methylsulfonyl)amino)-4,5,6,7-tetrahydro-1-benzothien-2-yl)-4-oxo-1,4-dihydro-3-quinolinecarboxylic acid

A solution of Example 83A (0.065 g, 0.148 mmol) in CH2C12 (5 ml) was cooled to 0° C., treated with diisopropylethylamine (0.029 g, 0.222 mmol) followed by methanesulfonyl chloride (0.022 g, 0.192 mmol) and stirred for 2 hours. The reaction mixture was partitioned between brine and CH2Cl2, the layers separated and the aqueous layer extracted with CH2Cl2. The combined organic phases were washed with brine and concentrated. The resulting residue was dissolved in 4:1 THF:water (5 ml), treated with LiOH-H20(0.031 g, 0.740 mmol) and stirred at room temperature for 2 hours. The mixture was diluted with saturated aqueous NH4Cl, brought to pH 5-6 with 10% H3PO4 and extracted several times with ethyl acetate. The combined organic phases were washed with brine, dried (Na2SO4), concentrated and the residue triturated in 25 % acetone in hexanes. Filtration gave the desired product as a tan solid (0.031 g, 43%). MS (DCI/NH₃) m/z 489 (M+H)⁺; ¹H NMR (300 MHz, DMSO-d6) δ 8.79 (s, 1H), 8.14 (d, 1H), 7.98 (d, 1H), 7.69 (s, 1H), 7.51 (d, 1H), 4.49 (m, 1H), 4.25 (m, 1H), 3.69 (s, 3H), 3.08 (s, 3H), 2.77 (m, 2H), 2.10-1.98 (m, 2H), 1.87-1.74 (m, 2H), 1.15 (m, 2H), 1.04 (m, 2H).

### EXAMPLE 89

### 1-cyclopropyl-8-methoxy-4-oxo-7-(4-(1H-pyrrol-1-yl)-4,5,6,7-tetrahydro-1-benzothien-2-yl)-1,4-dihydro-3-quinolinecarboxylic acid

A solution of Example 40 (0.07 g, 0.16 mmol), sodium acetate (0.07g, 0.94 mmol) and 2,5-dimethoxytetrahydrofuran (0.22 mL, 1.56 mmol) in acetic acid (1 mL) was heated at 80° C. for 2 hours. The reaction mixture was diluted with water (30 mL), filtered, and oven dried to yield (0.07 g, 97%) of the desired compound. MS (DCI/NH₃) m/z 461 (M+H)⁺; ¹H NMR (300 MHz, CD₃OD) δ 8.93 (s, 1H), 8.15 (d, 1H), 7.78 (d, 1H), 7.23 (s, 1H), 6.72 (m, 2H), 6.09 (m, 2H), 5.33 (m, 1H), 4.26 (m, 1H), 3.65 (s, 3H), 2.95 (m, 2H), 2.25 (m, 2H), 2.04 (m, 2H), 1.24 (m, 2H), 1.05 (m, 2H).

### EXAMPLE 90

### 1-cycloprpyl-7-((4E/Z1-4-(((ethylaminocarbothioyl)hydrazono)-4,5,6,7-tetrahydro-1-benzothien-2-yl)-8-methoxy-4-oxo-1,4-dihydro-3-quinolinecarboxylic acid

The desired product was prepared by substituting 4-ethyl-3-thiocarbazide, a reaction time of 48 hours and a reaction temperature of 100° C. for 1-aminopyrrolidine hydrochloride, a reaction time of 24 hours and a reaction temperature of 75° C. in Example 74. mp 265-266° C.; MS (APCI) m/z 511 (M+H)⁺; ¹H NMR (300 MHz, DMSO-d₆) δ 14.90 (s, 1H), 10.15 (s, 1H), 8.80 (s, 1H), 8.57 (dd, 1H), 8.48 (s, 1H), 8.15 (dd, 1H), 4.26 (m, 1H), 3.71 (s, 3H), 3.65 (q, 2H), 3.40 (s, 1H), 2.90 (dd, 2H), 2.70 (dd, 2H), 2.03 (dd, 2H), 1.18 (m, 2H), 1.16 (t, 3H), 1.06 (m, 2H).

### EXAMPLE 91

### 7-((4E/Z)-4-((amino(oxo)actyl)hydrazono)-4,5,6,7-4-tetrahydro-1-benzothien-2-yl)-1-cyclopropyl-8-methoxy-4-oxo-1,4-dihydro-3-quinolinecarboxylic acid

The desired product was prepared by substituting oxamic hydrazide, a reaction time of 18 hours and a reaction temperature of 100° C. in the absence of Et₃N for 1-aminopyrrolidine hydrochloride, a reaction time of 24 hours and a reaction temperature of 75° C. in Example 74. mp 298-300° C.; MS (APCI) m/z 493 (M-H)⁻, 495 (M+H)⁺; ¹H NMR (300 MHz, DMSO-d₆) δ 8.70 (s, 1H), 8.29 (br s, 2H), 8.13 (d, 1H), 8.05 (d, 1H), 8.01 (s, 1H), 7.96 (br s, 2H), 4.26 (m, 1H), 3.70 (s, 3H), 3.35 (s, 1H), 3.00 (dd, 2H), 2.71 (dd, 2H), 1.18 (m, 2H), 1.08 (m, 2H).

### EXAMPLE 92

### 1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylic acid

### EXAMPLE 92A

A solution of Example 50A (0.065 g, 0.15 mmol) and 1-amino-4-methylpiperazine (0.13 g, 1.2 mmol) in absolute ethanol (15 mL) was refluxed over 4A molecular sieves for 48 hour. The reaction mixture was concentrated and the residue purified by silica gel chromatography eluting with a gradient from CH₂Cl₂:CH₃OH (98:2) to CH₂Cl₂:CH₃0H (88:12) to provide the desired product (0.05 g, 63 %).

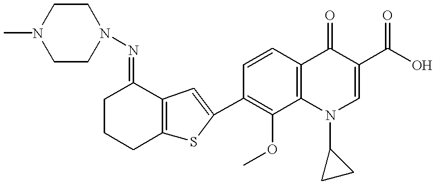

### EXAMPLE 92B

### 1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylic acid

A solution of Example 92A (0.20 g, 0.35 mmol) in of 3:1 THF:H₂ 0(10 mL) was treated with LiOH-H₂O (0.028 g, 0.070 mmol) and stirred overnight at room temperature. The reaction mixture was brought to pH˜5 with 10% HCl, was extracted with ethyl acetate, and the combined organic phases washed with brine, dried (Na₂SO₄) and concentrated to give the desired product (0.013 g, 68 %). MS (APCI) m/z 507 (M+H)⁺¹H NMR (300 MHz, CDCl₃), δ 8.85 (s, 1H) 8.18(d, 1H), 7.91 (s, 1H), 7.86 (d, 1H), 4.06 (m, 1H), 3.61 (s, 3H), 2.91-2.84 (m, 5H), 2.74-2.60 (m, 5H), 2.56 (m, 2H), 2.0 (t, 2H), 1.21 (d, 2H), 1.15 (s, 3H), 1.02 (s, 2H).

### EXAMPLE 94

### 1-cyclopropyl-8-methoxy-4-oxo-7-(4-((3-pyridinylmethyl)amino)-4,5,6,7-tetrahydro-1-benzothien-2-yl)-1,4-dihydro-3-quinolinecarboxylic acid dihydrochloride

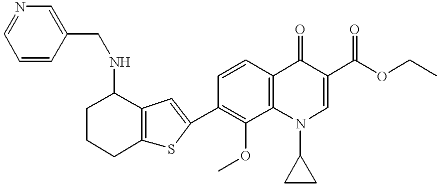

### EXAMPLE 94A

A solution of Example 83A (0.12g, 0.27 mmol) in dichloromethane (5 mL) was treated with acetic acid (1 drop), 3-pyridine carboxaldehyde (0.039 mL, 0.41 mmol), and sodium cyanoborohydride (0.021 g, 0.32 mmol) and stirred at room temperature for 2 hours. The reaction mixture was partitioned between 10% sodium bicarbonate solution and dichloromethane, dried (Na₂SO₄), concentrated and the residue purified by silica gel chromatography eluting with a gradient of 1% to 4% methanol in dichloromethane to yield (0.09 g, 62%) of the desired compound.

### EXAMPLE 94B

### 1-cyclopropyl-8-methoxy-4-oxo-7-(4-((3-pyridinulmethyl)amino)-4,5,6,7-tetrahydro-1-benzothien-2-yl)-1,4-dihydro-3-quinolinecarboxylic acid dihydrochloride The desired product was prepared by substituting Example 94A for Example 2A in Example 2B followed by treatment with 4M HCI in dioxane and filtration of the solid product. MS (DCI/NH₃) m/z 502 (M+H)⁺; ¹H NMR (300 MHz, CDCl₃) δ 8.90 (s, 1H), 8.67 (m, 1H), 8.53 (m, 1H), 8.23 (d, 1H), 7.88 (d, 1H), 7.57 (s, 1H), 6.58 (m, 1H), 4.11 (m, 1H), 3.96 (d, 2H), 3.87 (m, 1H), 3.66 (s, 3H0, 2.85 (m, 2H), 2.07 (m, 2H), 1.85 (m, 2H), 1.28 (m, 2H), 1.05 (m, 2H).

### EXAMPLE 96

### 7-((4E/Z)-4-(acetylhydrazono)-4,5,6,7-tetrahydro-1-benzothien-2-yl)-1-cyclopropyl-8-methoxy-4-oxo-1,4-dihydro-3-quinolinecarboxylic acid

The desired product was prepared by substituting acetic hydrazide, a reaction time of 60 hours and a reaction temperature of 100° C. in the absence of Et3N for 1-aminopyrrolidine hydrochloride, a reaction time of 24 hours and a reaction temperature of 75° C. in Example 74. mp 281-282° C.; MS (APCI) m/z 466 (M+H)⁺; ¹H NMR (300 MHz, DMSO-d₆) δ 8.80 (s, 1H), 8.14 (d, 1H), 8.00 (s, 1H), 7.95 (br s, 1H), 4.27 (m, 1H), 3.70 (s, 3H), 3.30 (s, 1H), 2.90 (m, 2H), 2.61 (m, 2H), 2.25 (s, 3H), 2.00 (m, 2H), 1.16 (m, 2H), 1.07 (m, 2H).

### EXAMPLE 97

### 7-(4-(benzylamino)-4,5,6,7-tetrahydro-1-benzothien-2-yl)-1-cyclopropyl-8-methoxn-4-oxo-1,4-dihydro-3-quinolinecarboxylic acid hydrochloride

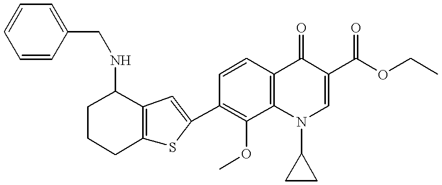

### EXAMPLE 97A

The desired product was prepared by substituting Example 83A and benzaldehyde for Example 83A and 3-pyridine carboxaldehyde, respectively in Example 94A.

### EXAMPLE 97B

### 7-(4-(benzylamino)-4,5,6,7-tetrahydro-1 -benzothien-2-yl]-1-cyclopropyl-8-methoxy-4-oxo-1,4-dihydro-3-quinolinecarboxylic acid hydrochloride

The desired product was prepared by substituting Example 97A for Example 94A in

### EXAMPLE 94B MS (DCI/NH₃) m/z 501 (M+H)⁺; ¹H NMR (300 MHz, DMSO-d₆) δ 8.80 (s, 1H), 8.17 (d, 1H), 8.13 (s, 1H), 7.97 (d, 1H), 7.65 (m, 2H), 7.44 (m, 3H), 4.51 (m, 1H), 4.26 (m, 3H), 3.70 (s, 3H), 2.89 (m, 2H), 2.15 (m, 2H), 1.86 (m, 2H), 1.17 (m, 2H), 1.04 (m, 2H).

### EXAMPLE 98

### 1-cyclopropyl-7-(4-(ethylamino)-4,5,6,7-tetrahydro-1-benzothien-2-yl)-8-methoxy-4-oxo-1,4-dihydro-3-quinolinecarboxylic acid hydrochloride

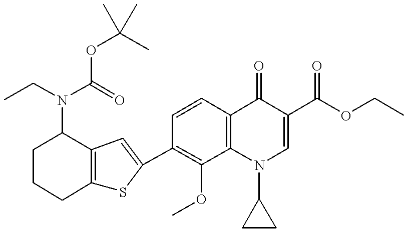

### EXAMPLE 98A

The desired product was prepared by substituting iodoethane for iodomethane in Example 77A.

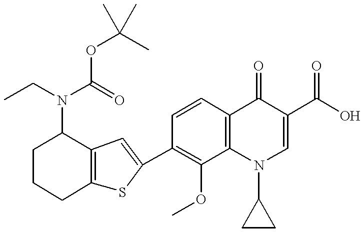

### EXAMPLE 98B

The desired product was prepared by substituting Example 98A for Example 2A in Example 2B.

### EXAMPLE 98C

### 1-cyclopropyl-7-(4-(ethylamino)-4,5,6,7-tetrahydro-1-benzothien-2-yl)-8-methoxy-4-oxo-1,4-dihydro-3-guinolinecarboxvlic acid hydrochloride

The desired product was prepared by substituting Example 98B for Example 40B in Example 40C. MS (DCI/NH₃) m/z 439 (M+H)⁺; ¹H NMR (300 MHz, CD₃OD) δ 8.98 (s, 1H), 8.24 (d, 1H), 7.95 (d, 1H), 7.81 (s, 1H), 4.52 (m, 1H), 4.30 (m, 1H), 3.72 (s, 3H), 3.26 (m, 2H), 2.98 (m, 2H), 2.00-2.24 (m, 4H), 1.40 (t, 3H), 1.28 (m, 2H), 1.08 (m, 2H).

### EXAMPLE 99

### 1-cyclopropyl-8-(difluoromethoxy)-7-(4-hydroxy-4,5,6,7-tetrahydro-1-benzothien-2-yl)-4-oxo-1,4-dihydro-3 -quinolinecarboxylic acid

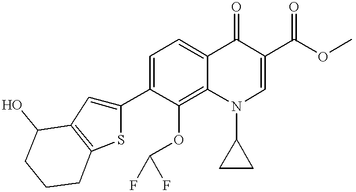

### EXAMPLE 99A

A solution of ethyl 1-cyclopropyl-7-bromo-8-difluoromethoxy-4-oxo-1,4-dihydroquinoline-3-carboxylate (1.50 g, 3.73 mmol) and Example 38B (3.12 g, 5.59 mmol) in toluene (40 ml) was treated with PdCl2(PPh3)2 (0.261 g, 0.373 mmol) and heated at 90° C. for 10 hours. The reaction mixture was allowed to cool, was poured into a mixture of 200 ml ethyl acetate and 300 ml saturated KF solution and stirred vigorously for 30 min. The biphasic mixture was filered through celite, the layers separated, the aqueous layer extracted with ethyl acetate, the compined organic phases washed with brine, dried (MgSO4). Concentration gave a residue that was passed through a plug of silica gel eluting with 25% acetone in hexanes and the filtrate and concentrated to give a mixture of carboxylate acid and ethyl ester.

A solution of this mixture in 1:1 MeOH:THF (30 ml) was treated with trimethylsilyldiazomethane (9.3 ml of a 2M solution in hexanes, 18.6 mmol)) and stirred at room temperature for 72 hours. The reaction mixture was partitioned between ethyl acetate and saturated NH4Cl solution, the organic phase washed with brine, dried (MgSO4) and concentrated to give the crude methyl ester as a yellow solid (1.80 g).

A solution of the crude product in THF (50 ml) was cooled to OoC, treated with TBAF (11.1 ml of a 1M solution in THF, 11.1 rmmol) and stirred for 2.5 hours. The reaction mixture was partitioned between ethyl acetate and saturated NH4Cl solution, the organic phase washed with brine, dried (MgSO4) and concentrated. The crude residue was purified by silica gel chromatography eluting with a gradient from 30% to 50% acetone in hexanes to give the desired product (0.83 g, 48%) as a yellow foam.

### EXAMPLE 99B

### 1-cyclopropyl-8-(difluoromethoxy)-7-(4-hydroxy-4,5,6,7-tetrahydro-1-benzothien-2-yl)-4-oxo-1,4-dihydro-3 -quinolinecarboxylic acid

The desired product was prepared by substituting Example 99A for Example 2A in Example 2B. The crude residue was triturated in 33% acetone in hexanes, filtered and washed with hexanes to give a yellow solid. MS (APCI) m/z 482 (M+H)⁺; ¹H NMR (300 MHz, DMSO-d₆) δ 14.67 (s, 1H), 8.85 (s, 1H), 8.27 (d, 1H), 7.95 (d, 1H), 7.69 (s, 1H), 6.94 (t, 1H), 5.19 (d, 1H), 4.62 (m, 1H), 4.15 (m, 1H), 2.85-2.65 (m, 2H), 2.05-1.86 (m, 2H), 1.83-1.61 (m, 2H), 1.20 (m, 2H), 1.03 (m, 2H).

### EXAMPLE 100

### 1-cyclopropyl-7-((4E/Z)-4-(4,5-dihydro-1H-imidazol-2-ylhydrazono)-4,5,6,7-tetrahydro-1-benzothien-2-yl)-8-methoxv-4-oxo-1,4-dihydro-3-quinolinecarboxylic acid

The desired product was prepared by substituting 2-hydrazino-2-imidazoline hyrdobromide, NaOAc and a reaction time of 48 hours for 1-aminopyrrolidine hydrochloride, Et3N and a reaction time of 24 hours in Example 74. mp 237-238° C.; MS (APCI) m/z 492 (M+H)⁺; ¹H NMR (300 MHz, DMSO-d₆) δ 9.25 (br s, 1H), 8.81 (s, 0.5H), 8.80 (s, 0.5H), 8.32 (s, 1H), 8.15 (d, 0.5H), 8.10 (d, 0.5H), 8.07 (d, 0.5H), 8.02 (d, 0.5H), 4.27 (m, 1H), 3.70 (d, 3H), 3.60 (m, 2H), 3.40 (m, 2H), 3.30 (s, 1H), 2.95 (dd, 1H), 2.90 (dd, 1H), 2.55 (dd, 1H), 2.00 (m, 2H), 1.28 (m, 2H), 1.04 (m, 2H).

### EXAMPLE 101

### 7-(4-amino-4,5,6,7-tetrahydro-1-benzothien-2-yl)-1-cyclopropyl-8-(difluoromethoxy)-4-oxo-1,4-dihydro-3-quinolinecarboxylic acid hydrochloride

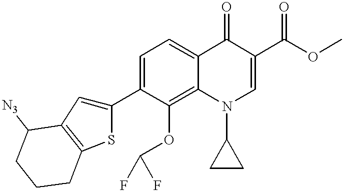

### EXAMPLE 101A

The desired product was prepared by substituting Example 99A for Example 37A in Example 37B and the crude product purified by silica gel chromatography eluting with a gradient from 25% to 50% acetone in hexanes.

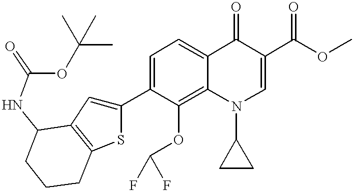

### EXAMPLE 101B

The desired product was prepared by substituting Example lOlA for Example 201C in Example 201C and the crude product triturated in hot 25% acetone in hexanes and filtered.

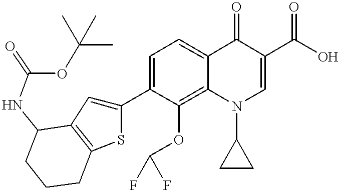

### EXAMPLE 101C

The desired product was prepared by substituting Example lO1B for Example 2A in Example 2B.

### EXAMPLE 101D

### 7-(4-amino-4,5,6,7-tetrahydro-1-benzothien-2-yl)-1-cyclopropyl-8-(difluoromethoxy)-4-oxo-1,4-dihydro-3-quinolinecarboxylic acid hydrochloride

The desired product was prepared by substituting Example 101C for Example 40B in Example 40C. MS (APCI) m/z 481 (M+Cl)⁻; ¹H NMR (300 MHz, DMSO-d₆) δ 8.87 (s, 1H), 8.52 (br s, 3H), 8.34 (d, 1H), 8.01 (s, 1H), 7.88 (d, 1H), 7.02 (t, 1H), 4.42 (m, 1H), 4.15 (m, 1H), 2.86 (m, 2H), 2.09-1.97 (m, 2H), 1.93-1.77 (m, 2H), 1.20 (m, 2H), 1.04 (m, 2H).

### EXAMPLE 102

### 1-cyclopropyl-7-(4-hydroxy-2-methyl-1,1-dioxido-3,4-dihydro-2H-thieno[3,2-e][1,2]thiazin-6-yl)-8-methoxy-4-oxo-1,4-dihydro-3-quinolinecarboxylic acid

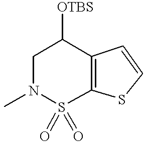

### EXAMPLE 102A

The desired product was prepared by substituting 4-Hydroxy-3,4-dihydro-2-methyl-2H-thieno[3,2-e]1,2-thiazine 1,1 -dioxide (prepared by the method of DuPriest, et.al. J. Org. Chem. 1997, 62, 9372-75) for the intermediate alcohol in Example 218B.

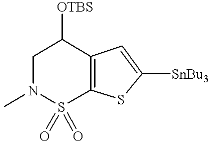

### EXAMPLE 102B

A solution of diisopropyl amine (0.412 g, 4.08 mmol) in THF (50 mL) was cooled to 0° C., treated dropwise with n-BuLi (1.5 mL of a 2.5 M solution in hexanes, 3.75 mmol), stirred for 30 minutes and cooled to −50° C. This solution was treated dropwise with a solution of Example 102A (0.543 g, 1.63 mmol) in THF (10 mL), stirred for 1 hour at 0° C., recooled to −50° C., treated with chlorotributylstannane (0.584 g, 1.79 mmol) and allowed to warm to room temperature overnight. The reaction mixture was partitioned between water and ethyl acetate, the aqueous phase extracted with ethyl acetate, the combined organic phases washed with water, brine, dried (Na₂SO₄) and concentrated to provide the desired product that was used without purification.

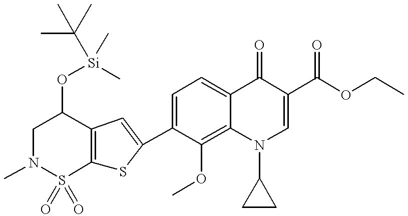

### EXAMPLE 102C

A solution of Example 102B (0.37 g, 0.60 mmol), ethyl 7-bromo-1-cyclopropyl-8-methoxy-4-oxo-1,4-dihydro-3-quinolinecarboxylate (0.220 g, 0.60 mmol) and PdCl2(PPh3)2 (0.042 g, 0.06 mmol) in toluene (2 ml) was heated to 85° C. for 6 hours. The reaction mixture was allowed to cool, concentrated, suspended in ethyl acetate and filtered. The filtrate was concentrated and purified by silica gel chromatography eluting with 20% acetone in hexanes to give the desired product (0.173 g, 47%) as a colorless solid.

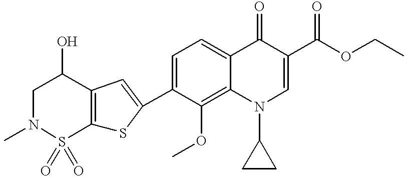

### EXAMPLE 102D

The desired product was prepared by substituting Example 102C for Example 35D in Example 35E and the crude residue purified by silica gel chromatography eluting with 30% then 50% acetone in hexanes.

### EXAMPLE 102E

### 1-cyclopropyl-7-(4-hydroxy-2-methyl-1,1-dioxido-3,4-dihydro-2H-thieno[3,2-e][1,2]thiazin-6-yl)-8-methoxy-4-oxo-1 4-dihydro-3-quinolinecarboxylic acid

The desired product was prepared by substituting Example 102D for Example 48C in Example 48D. MS (DCI/NH₃) m/z 477 (M+H)⁺; ¹H NMR (300 MHz, DMSO-d₆) δ 14.77(s, 1H), 8.83 (s, 1H), 8.20 (d, 1H), 8.14 (d, 1H), 7.97 (s, 1H), 6.05 (d, 1H), 4.92 (q, 1H), 4.27 (m, 1H), 3.94 (dd, 1H), 3.77 (s, 3H), 3.73 (dd, 1H), 2.97 (s, 3H), 1.18 (m, 2H), 1.05 (m, 2H).

### EXAMPLE 104

### 1-cyclopropyl-7-(4-((3-fluorobenzyl)amino)-4,5,6,7-tetrahydro-1-benzothien-2-yl)-8-methoxy-4-oxo-1,4-dihydro-3-quinolinecarboxylic acid hydrochloride

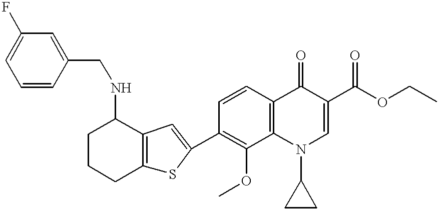

### EXAMPLE 104A

The desired product was prepared by substituting Example 3-fluorobenzaldehyde for 3-pyridine carboxaldehyde in Example 94.

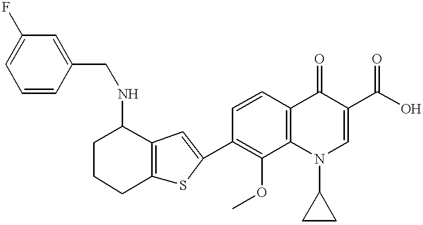

### EXAMPLE 104B

### 1-cyclopropyl -7-(4-((3-fluorobenzyl)amino)-4,5,6,7-tetrahydro-1-benzothien-2-yl)-8-methoxy-4-oxo-1,4-dihydro-3-quinolinecarboxylic acid hydrochloride

The desired product was prepared by substituting Example 104A for Example 94A in Example 94B. MS (DCI/NH₃) m/z 519 (M+H)⁺; ¹HNMR (300 MHz, CD₃OD) δ 8.97 (s, 1H), 8.23 (d, 1H), 7.93 (d, 1H), 7.85 (s, 1H), 7.51 (m, 1H), 7.48 (m, 2H), 7.22 (m, 1H), 467 (m, 1H), 4.43 (m, 2H), 4.29 (m, 1H), 3.72 (s, 3H), 3.01 (m,2H), 2.00-2.32 (m, 4H), 1.28 (m, 2H), 1.08 (m, 2H).

### EXAMPLE 105

### 7-((4E/Z)-4-[(aminocarbothioyl)(methyl)hydrazono)-4,5,6,7-tetrahydro-1-benzothien-2-yl)-1-cyclopropyl-8-methoxy-4-oxo-1 4-dihydro-3-quinolinecarboxylic acid

The desired product was prepared by substituting 2-methylthiocarbazide, a reaction time of 60 hours and a reaction temperature of 100° C. for 1-aminopyrrolidine hydrochloride, a reaction time of 24 hours and a reaction temperature of 75° C. in Example 74. mp 176-178° C.; MS (APCI) m/z 497 (M+H)⁺; ¹H NMR (300 MHz, DMSO-d₆) δ 8.65 (s, 1H), 8.25 (d, 1H), 7.90 (s, 1H), 7.62 (d, 1H), 4.26 (m, 1H), 4.00 (s, 2H), 3.70 (s, 3H), 3.30 (s, 1H), 3.00 (dd, 2H), 2.62 (dd, 2H), 2.00 (m, 2H), 1.40 (s, 3H), 1.18 (m, 2H), 1.03 (m, 2H).

### EXAMPLE 106

### 1-cyclopropyl-8-methoxy-7-(5-methyl-4-oxo-4,5,6,7-tetrahydro-1-benzothien-2-yl)-4-oxo-1,4-dihydro-3-quinolinecarboxylic acid

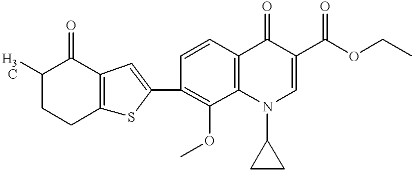

### EXAMPLE 106A

A solution of Example 86A (0.50 g, 0.0011 mol) in ethanol (80 mL) in a Paar apparatus was treated with 10% Pd/C (0.050 g, 10 wt%), pressurized to 60 psi and heated to 65° C. When the reaction was determined complete based upon H2 consumption, the mixture was allowed to cool, was filtered through celite and concentrated to give the desired product (0.50 g, 92%) as a solid which was used without further purification.

### EXAMPLE 106B

### 1-cyclopropyl-8-methoxy-7-(5-methyl-4-oxo-4,5,6,7-tetrahydro-1-benzothien-2-yl)-4-oxo-1,4-dihydro-3-quinolinecarboxylic acid

The desired product was prepared by substituting Example 106A for Example 2A in Example 2B to give the desired product. MS (APCI) m/z 424 (M+H)⁺¹H NMR (300 MHz, CDCl₃) δ 14.66 (s, 1H), 8.93 (s, 1H), 8.29 (d, 1H), 7.94 (s, 1H), 7.79 (d, 1H), 4.11 (m, 1H), 3.68 (s, 3H), 3.18-3.12 (m, 2H), 2.63 (m, 1H), 2.39-2.28 (m, 1H), 2.05 (m, 1H), 1.31 (d, 3H), 1.08 (s, 2H), 0.86 (s, 2H).

### EXAMPLE 107

### 1-cyclopropyl-8-(difluoromethoxy)-7-(4-(methylamino)-4,5,6,7-tetrahydro-1-benzothien-2-yl)-4-oxo-1,4-dihydro-3-quinolinecarboxylic acid hydrochloride

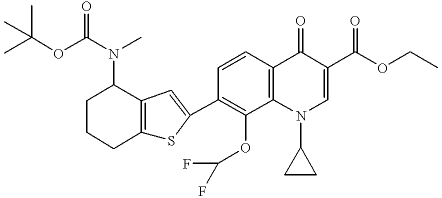

### EXAMPLE 107A

The desired product was prepared by substituting Example 210B and ethyl 7-bromo-1-cyclopropyl-8-difluoromethoxy-4-oxo-1,4-dihydro-3-quinolinecarboxylate and a reaction time of 3 hours for Example 1D, ethyl 7-bromo-1-cyclopropyl-4-oxo-1,4-dihydro-3-quinolinecarboxylate and a reaction time of 24 hours in Example 1E.

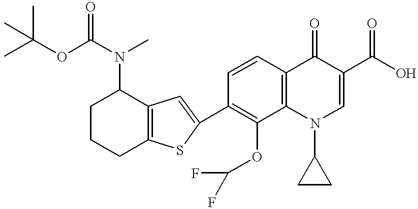

### EXAMPLE 107B

The desired product was prepared by substituting Example 107A for Example 2A in Example 2B.

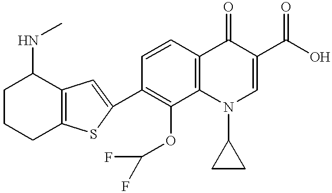

### EXAMPLE 107C

### 1-cyclopropyl-8-(difluoromethoxy)-7-(4-(methylamino)-4,5,6,7-tetrahydro-1-benzothien-2-yl)-4-oxo-1,4-dihydro-3-quinolinecarboxylic acid hydrochloride

The desired product was prepared by substituting Example 107B for Example 40B in Example 40C. MS (DCI/NH₃) m/z 461 (M+H)⁺; ¹H NMR (300 MHz, CD₃OD) δ 8.98 (s, 1H), 8.40 (d, 1H), 7.87 (d, 1H), 7.72 (s, 1H), 6.62 (t, 1H), 4.45 (m, 1H), 4.26 (m, 1H), 2.99 (m, 2H), 2.80 (s, 3H), 2.00-2,27 (m, 4H), 1.32 (m, 2H), 1.07 (m, 2H).

### EXAMPLE 108

### 1-cyclopronyl-6-fluoro-7-(4-(methylamino)-4,5,6,7-tetrahydro-1-benzothien-2-yl)-4-oxo-1,4-dihydrol,1,8]naphthyridine-3-carboxylic acid hydrochloride

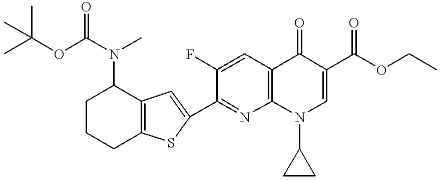

### EXAMPLE 108A

The desired product was prepared by substituting Example 210B, ethyl 1-cyclopropyl-7-chloro-4-oxo-1,4-dihydro[1,8]naphthyridine-3-carboxylate and a reaction time of 3 hours for Example 1D, ethyl 7-bromo-1-cyclopropyl-4-oxo-1,4-dihydro-3-quinolinecarboxylate and a reaction time of 24 hours in Example 1E.

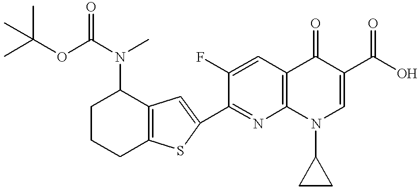

### EXAMPLE 108B

The desired product was prepared by substituting Example 108A for Example 2A in Example 2B.

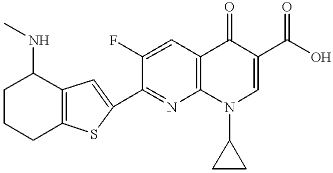

### EXAMPLE 108C

### 1-cyclopropyl-6-fluoro-7-(4-(methylamino)-4,5,6,7-tetrahydro-1-benzothien-2-yl)-4-oxo-1,4-dihydro[1,8]naphthyridine-3-carboxylic acid hydrochloride

The desired product was prepared by substituting Example 108B for Example 40B in Example 40C. MS (DCI/NH₃) m/z 414 (M+H)⁺; ¹H NMR (300 MHz, CD₃OD) δ 8.96 (s, 1H), 8.49 (d, 1H), 8.13 (s, 1H), 4.50 (m, 1H), 3.87 (m, 1H), 3.02 (m, 2H), 2.82 (s, 3H), 1.98-2.30 (m, 4H), 1.41 (m, 2H), 1.20 (m, 2H).

### EXAMPLE 109

### 7-(4-amino-2-methyl-1,1-dioxido-3,4-dihydro-2H-thieno[3,2-e][1,2]thiazin-6-yl)-1-cyclopropyl-8-methoxy-4-oxo-1,4-dihydro-3-quinolinecarboxylic acid hydrochloride

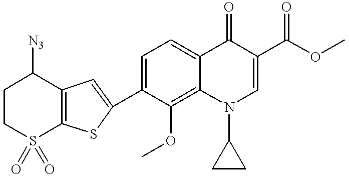

### EXAMPLE 109A

The desired product was prepared by substituting Example 102D for Example 206E in Example 206F and the crude residue purified by silica gel chromatography eluting with 30% then 50% acetone in hexanes.

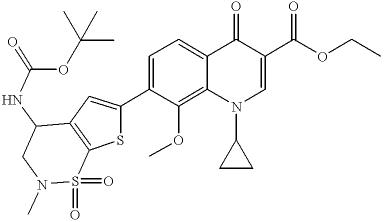

### EXAMPLE 109B

The desired product was prepared by substituting Example 109A for Example 201C in Example 201C and the crude residue purified by silica gel chromatography eluting with 30% acetone in hexanes.

### EXAMPLE 109C

### 7-(4-amino-2-methyl-1,1-dioxido-3,4-dihydro-2H-thieno[3,2-e][1,2]thiazin-6-yl)-1-cyclopropyl-8-methoxy-4-oxo-1,4-dihydro-3-quinolinecarboxylic acid hydrochloride

The desired product was prepared by substituting Example 109B for Example 48C in Example 48D. MS (DCI/NH₃) m/z 476 (M+H)⁺; ¹H NMR (300 MHz, DMSO-d₆) δ 8.85(s, 1H), 8.35 (s, 1H), 8.29 (d, 1H), 7.95 (m, 1H), 4.96 (dd, 1H), 4.28 (m, 1H), 4.03 (m, 2H), 3.79 s, 3H), 2.97 (s, 3H), 1.18 (m, 2H), 1.04 (m, 2H).

### EXAMPLE 110

### 1-cyclopropyl-7-(4-(hydroxymethyl)-4,5,6,7-tetrahydro-1-benzothien-2-yl)-8-methoxy-4-oxo-1,4-dihydro-3 -quinolinecarboxylic acid

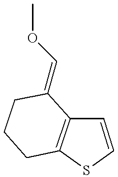

### EXAMPLE 110A

To a stirring suspension of (methoxymethyl)triphenylphosphonium chloride (26.105 g, 76.15 mmol) in diethyl ether at 0° was added phenyl lithium (76.15 mmol) dropwise from an addition fuinnel over 15 minutes. The resulting orange suspension was stirred at 0° for 1 hour before cooling to −78° and adding a solution of the 4-keto-4,5,6,7-tetrahydrothianaphthene (11.04 g, 72.53 mmol) in diethyl ether dropwise over 35 minutes. The reaction mixture was stirred overnight while gradually warming to room temperature. The reaction was quenched with water and extracted into 3×CH₂Cl₂, the combined organic layers dried (Na₂SO₄), concentrated to give a colorless oil (12.36 g, 95% yield) as a mixture of geometric isomers.

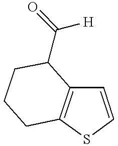

### EXAMPLE 110B

A solution of Example 110A (6.02 g, 33.40 mmol) in formic acid (100 mL) was stirred at room temperature for 3.5 hours. The reaction mixture was poured over sat. NaHICO₃, neutralized by the addition of solid NaHCO₃ , partitioned with CH₂CI₂ and the organic phase dried (MgSO₄) Concentration gave an oil that was used without further purification.

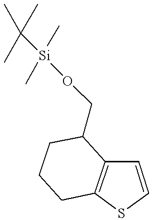

### EXAMPLE 110C

The desired product was prepared by substituting Example 1 lOB for Example 218A in Example 218B and was purified by silica gel chromatography eluting with hexane to 5% ether in hexane step gradient.

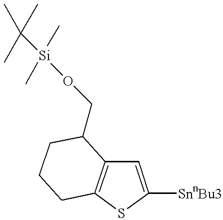

### EXAMPLE 110D

The desired product was prepared by substituting Example 110C for Example 218B in Example 218C.

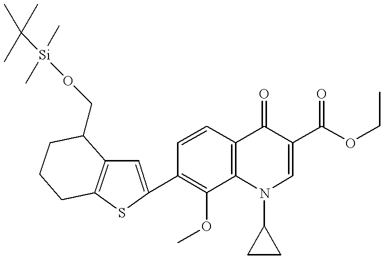

### EXAMPLE 110E

The desired product was prepared by substituting Example 110D and ethyl 7-bromo-1-cyclopropyl-8-methoxy-4-oxo-1,4-dihydro-3-quinolinecarboxylate for Example 1D and 7-bromo-1-cyclopropyl-4-oxo-1,4-dihydro-3-quinolinecarboxylate, respectively in Example 1E.

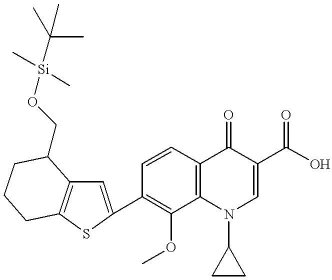

### EXAMPLE 110F

The desired product was prepared by substituting Example 110E for Example 2A in Example 2B.

### EXAMPLE 110G

### 1-cyclopropyl-7-(4-(hydroxymethyl)-4,5,6,7-tetrahydro-1-benzothien-2-yl)-8-methoxy-4-oxo-1,4-dihydro-3-quinolinecarboxylic acid

The desired product was prepared by substituting Example 110F for Example 35D in Example 35E and was purified by silica gel chromatography eluting with 20%-50% acetone in hexanes. The resulting yellow solid was dissolved in a minimum of CH₂C1₂, triturated with Et₂O, filtered, washed with Et₂O and dried under vacuum to give a white solid. MS (DCI/NH₃) m/z 426 (M+H)⁺; ¹H-NMR (300 MHz, d₆-DMSO) δ 14.05 (s, 1H), 8.78 (s, 1H), 8.10 (d, 1H), 8.00 (d, 1H), 4.75 (dd, 1H), 4.25 (m, 1H), 3.70 (m, 1H), 3.67 (s, 3H), 3.49 (m, 1H), 2.77 (m, 3H), 1.97-1.63 (m, 5H), 1.15 (m, 2H), 1.04 (m, 2H).

### EXAMPLE 111

### 1-cyclopropyl-8-methoxy-4-oxo-7-(4-(1-pyrrolidinylmethyl)-4,5,6,7-tetrahydro-1-benzothien-2-yl)-1,4-dihydro-3-guinolinecarboxvlic acid

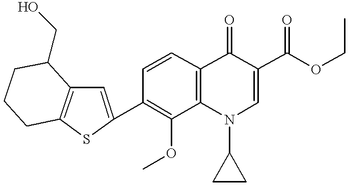

### EXAMPLE 111A

The desired product was prepared by substituting Example 110E for Example 35D in Example 35E and was used without further purification.

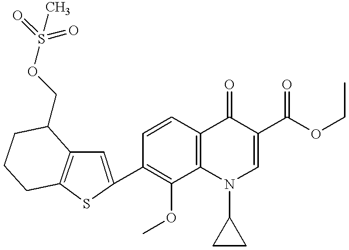

### EXAMPLE 111B

The desired product was prepared by substituting Example 111A for Example 45G in Example 342124A and was used without further purification.

### EXAMPLE 111C

### 1-cyclopropyl-8-methoxy-4-oxo-7-(4-(1-pyrrolidinylmethyl)-4,5,6,7-tetrahydro-1-benzothien-2-yl)-1,4-dihydro-3-quinolinecarboxylic acid

A solution of Example 111B (0.050 g, 0.094 mmol), pyrrolidine (85 uL, 0.94 mmol) in THF (2 mL) was heated at 50° overnight in a sealed vial. The reaction mixture was partitioned between ethyl acetate and 1 N HCl and the organic phase was removed. The aqueous phase was adjusted to pH=7.5 with 1N NaOH, was extracted with ethyl acetate, the combined organic phases dried (Na₂SO₄) and concentrated to give the intermediate ester a colorless solid (0.039 g, 82% yield)

A solution of the ester above in 1N HCl (5 mL) was heated at 80° for 4 hours, cooled to room temperature and concentrated. The residue was triturated in methanol and with diethyl ether, filtered and the solid product washed with diethyl ether and dried under vacuum to give the desired product (0.023 g, 65% yield) as a white solid. MS (DCINH₃) m/z 479 (M+H)⁺; ¹H-NMR (300 MHz, d₆-DMSO) δ 9.35 (br s, 1H), 8.79 (s, 1H), 8.13 (d, 1H), 7.97 (d, 1H), 7.70 (s, 1H), 4.25 (m, 1H), 3.68 (s, 3H), 3.50-3.00 (m, 1OH), 2.80 (m, 1H), 2.20-1.70 (m, 6H), 1.15 (m, 2H), 1.03 (m, 2H).

### EXAMPLE 112

### 1-cyclopropyl-8-methoxy-4-oxo-7-(4-((2-pyrrolidinylmethyl)amino)-4,5,6,7-tetrahydro-1-benzothien-2-yl)-1,4-dihydro-3-quinolinecarboxylic acid dihydrochloride

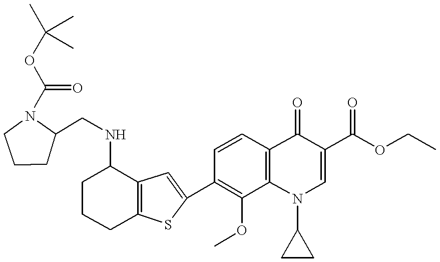

### EXAMPLE 112A

The desired product was prepared by substituting N-(tert-Butoxycarbonyl)-prolinal for 3-pyridine carboxaldehyde in Example 94A.

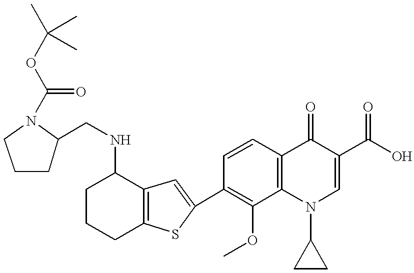

### EXAMPLE 112B

The desired product was prepared by substituting Example 1 12A for Example 2A in Example 2B.

### EXAMPLE 112C

### 1-cyclopropyl-8-methoxy-4-oxo-7-(4-((2-pyrrolidinylmethyl)amino)-4,5,6,7-tetrahydro-1-benzothien-2-yl)-1,4-dihydro-3-quinolinecarboxylic acid dihydrochloride

The desired product was prepared by substituting Example 112B for Example 40B in Example 40C. MS (DCI/NH₃) m/z 494 (M+H)⁺; ¹H NMR (300 MHz, CD₃OD) δ 8.97 (s, 1H), 8.23 (d, 1H), 8.08 (s 1H), 8.02 (d, 1H), 4.67 (m, 1H), 4.29 (m, 1H), 4.02 (m, 1H), 3.72 (s, 3H), 3.61 (m, 1H), 3.43 (m, 2H0, 3.32 (m, 2H), 3.00 (m, 2H), 2.40 (m, 1H), 2.03-2.28 (m, 6H), 1.93 (m, 1H), 1.28 (m, 2H), 1.07 (m, 2H).

### EXAMPLE 113

### 7-(4-(acetylamino)-4,5,6,7-tetrahydro-1-benzothien-2-yl)-1-cyclopropyl-8-methoxy-4-oxo-1,4-dihydro-3-quinolinecarboxylic acid

An Argonaut Quest 210 synthesizer was equipped with 5 ml reaction tubes. One tube was charged with 1 ml of 1:1/dichloromethane:dimethylacetamide, N-cyclohexylcarbodiimide, N′-methyl polystyrene HL (NovaBiochem, 200-400 mesh, 2% DVB, 1.52 mmol/g loading) (0.151 g, 0.23 mmol), 1-hydroxybenzotriazole hydrate (0.003 g, 0.019 mmol) and acetic acid (0.01 ml, 0.17 mmol) and the mixture was agitated for 30 min. A solution of Example 83A (0.050 g, 0.114 mmol) in Iml of 1:1/dichloromethane:dimethylacetamide was added and the mixture was stirred for 48 h at room temperature. PS-Trisamine resin (Argonaut, 100-200 mesh, 1% DVB, 4.06 mmol/g loading) (0.135 g, 0.55 mmol) was added and the mixture was agitated for 2 h. The mixture was filtered through the Quest reaction tube and the residual resin was washed with dichloromethane (2×2 ml). The combined filtrates were concentrated and eluted through an Extract-Clean Silica Tube (Alltech, 2 g, 12 ml) with 2% methanol in dichloromethane. The product fraction was collected, concentrated to a volume of ca. 2 ml and was washed with 2N aqueous sodium hydroxide solution (1×2 ml). The layers were separated and the organic layer was evaporated to dryness. The residue was redissolved in 1:1/tetrahydrofuran:methanol (2 ml), 2N aqueous sodium hydroxide solution (0.83 ml, 1.66 mmol) was added and the mixture was shaken at ambient temperature over night. The mixture was neutralized with 4M hydrochloric acid in dioxane and was concentrated to dryness The residue was taken up in 1:1/dichloromethane:methanol (2 ml), filtered and the filtrate was evaporated to dryness. The residue was purified by preparative reverse phase HPLC (Waters Prep Nova-Pak HR C18 column, 25×100 mm, 6 μ, 60 Å, 0.01% trifluoroacetic acid in water:acetonitrile) to give the desired product (0.045 g, 89%) as an off-white solid. MS (DCI/NH₃) m/z 453 (M+H)⁺; ¹H NMR (300 MHz, DMSO d₆) δ 8.78 (s, 1H), 8.25 (d, 1H), 7.98 (d, 1H), 7.56 (s, 1H), 4.92 (m, 1H), 4.26 (m, 1H), 3.68 (s, 3H), 2.78 (m, 2H), 1.94 (m, 1H), 1.88 (s, 3H), 1.82 (m, 1H), 1.67 (m, 2H), 1.16 (m, 2H), 1.10 (m, 2H).

### EXAMPLE 114

### 1-cyclopropyl-8-methoxy-4-oxo-7-(4-(propionylamino)-4,5,6,7-tetrahydro-1-benzothien-2-yl)-1,4-dihydro-3-quinolinecarboxylic acid

The desired product was prepared by substituting propionic acid for acetic acid in Example 113. MS (DCI/NH₃) m/z 467 (M+H)⁺; ¹H NMR (300 MHz, DMSO-d₆) δ 8.78 (s, 1H), 8.18 (d, 1H), 8.10 (d, 1H), 7.95 (d, iH), 7.54 (s, 1H), 4.94 (m, 1H), 4.25 (m, 1H), 3.68 (s, 3H), 2.80 (m, 2H), 2.16 (q, 2H), 1.92 (m, 2H), 1.81 (m, 1H), 1.66 (m, 1H), 1.15 (m, 2H), 1.05 (t, 2H), 1.04 (m, 3H).

### EXAMPLE 115

### 1-cyclopropyl-8-methoxy-7-(4-((methoxyacetyl)amino)-4,5,6,7-tetrahydro-1-benzothien-2-yl)-4-oxo-1,4-dihydro-3-quinolinecarboxylic acid

The desired product was prepared by substituting 2-methoxyacetic acid for acetic acid in Example 113. MS (DCI/NH₃) m/z 483 (M+H)⁺; ¹H NMR (300 MHz, DMSO-d₆) δ 8.78 (s, 1H), 8.10 (d, 1H), 9.06 (d, 1H), 7.96 (d, 1H), 7.52 (s, 1H), 5.02 (m, 1H), 4.25 (m, 1H), 3.88 (s, 2H), 3.68 (s, 3H), 3.32 (s, 3H), 2.80 (m, 2H), 1.71-2.30 (m, 4H), 1.16 (m, 2H), 1.03 (m, 2H).

### EXAMPLE 116

### 1-cyclopropyl-8-methoxy-4-oxo-7-(4-((tetrhydrofuranyl-2-carbonyl)amino)-4,5,6,7-tetrahydro-1-benzothien-2-yl)-1,4-dihydro-3-quinolinecarboxylic acid

The desired product was prepared by substituting 2-tetrahydrofuroic acid for acetic acid in Example 113. MS (DCI/NH₃) m/z 509 (M+H)⁺; ¹H NMR (300 MHz, DMSO-d₆) δ 8.78 (s, 1H), 8.10 q, 1H), 7.94 (d, 1H), 4.96 (m, 1H), 4.22-4.43 (m, 2H), 3.90 (m, 1H), 3.70-3.82 (m, 2H), 3.68 (d, 2H), 3.64 (s, 3H),2.80 (m, 2H), 2.16 (m, 1H), 1.25-2.03 (m, 6H), 1.15 (m, 2H), 1.04 (m, 2H).

### EXAMPLE 117

### 1-cyclopropyl-8-methoxy-4-oxo-7-(4-((tetrahydrofuranyl-3-carbonyl)amino)-4,5,6,7-tetrahydro-1-benzothien-2-yl)-1,4-dihydro-3 -quinolinecarboxylic acid

The desired product was prepared by substituting 3-tetrahydrofuroic acid for acetic acid in Example 113. MS (DCI/NH₃) m/z 509 (M+H)⁺; ¹H NMR (300 MHz, DMSO-d₆) δ 8.79 (s, 1H), 8.36 (d, 1H), 8.10 (dd, 1H), 7.92 (dd, 1H), 7.53 (d, 1H), 4.96 (m, 1H), 4.26 (m, 1H), 3.61-3.91 (m, 7H), 2.81 (m, 2H), 1.90-2.13 (m, 3H), 1.84 (m, 1H), 1.68 (m, 1H), 1.18 (m, 2H), 1.04 (m, 2H).

### EXAMPLE 118

### 1-cyclopropyl-8-methoxy-7-(4-((4-morpholinylacetyl)amino)-4,5,6,7-tetrahydro-1-benzothien-2-vl)-4-oxo-1,4-dihydro-3-quinolinecarboxylic acid

The desired product was prepared by substituting 2-morpholinoacetic acid for acetic acid in Example 113. MS (DCI/NH₃) m/z 538 (M+H)⁺; ¹H NMR (300 MHz, DMSO-d₆) δ 9.11 (d, 1H), 8.79 (s, 1H), 8.11 (d, 1H), 8.04 (d, 1H), 7.64 (s, 1H), 5.02 (m, 1H), 4.26 (m, 1H), 4.04 (s, 2H), 3.86-3.98 (m, 6H), 3.68 (s, 2H), 3.49 (m, 2H), 3.28 (m, 2H), 2.82 (m, 2H), 2.00 (m, 2H), 1.86 (m, 1H), 1.74 (m, 1H), 1.17 (m, 2H), 1.03 (m, 2H).

### EXAMPLE 119

### 1-cyclopropyl-8-methoxy-7-(4-((3-(4-morpholinyl)propanoyl)amino)-4,5,6,7-tetrahydro-1-benzothien-2-yl)-4-oxo-1,4-dihydro-3-quinolinecarboxylic acid

The desired product was prepared by substituting 3-morpholinopropionic acid for acetic acid in Example 113. MS (DCI/NH₃) m/z 552 (M+H)⁺, ¹H NMR (300 MHz, DMSO-d₆) δ 11.41 (br s, 1H), 8.79 (s, 1H), 8.59 (d, 1H), 8.11 (d, 1H), 7.99 d, 1H), 7.59 (s, 1H), 4.96 (m, 1H), 4.25 (m, 1H), 3.78-3.99 (m, 4H), 3.68 (s, 3H), 3.48 (m, 4H), 3.08 (m, 2H), 2.79 (m, 2H), 1.94 (m, 2H), 1.84 (m, 1H), 1.70 (m, 1H), 1.16 (m, 2H), 1.03 (m, 2H).

### EXAMPLE 120

### 1-cyclopropyl-8-methoxy-4-oxo-7-(4-((1H-pyrrol-2-ylcarbonyl)amino)-4,5,6,7-tetrahydro-1-benzothien-2-yl)-1,4-dihydro-3-quinolinecarboxylic acid

The desired product was prepared by substituting pyrrole-2-carboxylic acid for acetic acid in Example 113. MS (DCI/NH₃) m/z 504 (M+H)⁺; ¹H NMR (300 MHz, DMSO-d₆) δ 11.49 (br s, 1H), 8.77 (s, 1H), 8.30 (d, 1H), 8.08 (d, 1H), 7.93 (d, 1H), 7.56 (s, 1H), 6.87 (m, 2H), 6.07 (m, 1H), 5.18 (m, 1H), 4.24 (m, 1H), 3.68 (s, 3H), 2.82 (m, 2H), 1.75-2.10 (m, 4H), 1.15 (m, 2H), 1.02 (m, 2H).

### EXAMPLE 121

### 1-cyclopropyl-8-methoxy-4-oxo-7-(4-((3-pyridinylacetyl)amino)-4,5,6,7-tetrahydro-1-benzothien-2-yl)-1,4-dihydro-3-quinolinecarboxylic acid

The desired product was prepared by substituting 3-pyridyl-acetic acid for acetic acid in Example 113. MS (DCI/NH₃) m/z 530 (M+H)⁺; ¹H NMR (300 MHz, DMSO-d₆) δ 8.93 (s, 1H), 8.8 (d, 1H), 8.83 (d, 1H), 8.79 (s, 1H), 8.59 (d, 1H), 8.10 (d, 1H), 8.09 (dd, 1H), 7.94 (d, 1H), 7.54 (s, 1H), 4.96 (m, 1H), 4.28 (m, 1H), 3.88 (d, 1H), 3.68 (s, 3H), 2.82 (m, 1H), 1.84 (m, 2H), 1.72 (m. 1H), 1.17 (m, 2H), 1.02 (m, 2H).

### EXAMPLE 122

### 1-cyclopropyl-8-methoxy-4-oxo-7-(4-((3-pyridazinylcarbonyl)amino)-4,5,6,7-tetrahydro-1-benzothien-2-yl)-1,4-dihydro-3-quinolinecarboxylic acid

The desired product was prepared by substituting pyridazine-3-carboxylic acid for acetic acid in Example 113. MS (DCI/NH₃) m/z 517 (M+H)⁺; ¹H NMR (300 MHz, DMSO-d₆) δ 9.43 (dd, 1H), 9.31 (d, 1H), 8.77 (s, 1H), 8.31 (dd, 1H), 8.05 (d, 1H), 7.99 (dd, 1H), 7.93 (d, 1H), 7.63 (s, 1H), 5.28 (m, 1H), 4.25 (m, 1H), 3.68 (s, 3H), 2.86 (m, 2H), 1.83-2.14 (m, 4H), 1.15 (m, 2H), 1.04 (m, 2H).

### EXAMPLE 123

### 1-cyclopropyl-7-(4-((1H-imidazol-2-ylcarbonUl)amino)-4,5,6,7-tetrahydro-1-benzothien-2-yl)-8-methoxy-4-oxo-1,4-dihydro-3-quinolinecarboxylic acid

The desired product was prepared by substituting imidazole-2-carboxylic acid for acetic acid in Example 113. MS (DCI/NH₃) m/z 505 (M+H)⁺; ¹H NMR (300 MHz, DMSO-d₆) δ 9.80 (d, 1H), 8.78 (s, 1H), 8.08 (d, 1H), 7.98 (d, 1H), 7.80 (s, 2H), 7.70 (s, 1H), 5.70 (m, 1H), 4.25 (m, 1H), 3.69 (s, 3H), 2.85 (m, 2H), 1.84-2.16 (m, 4H), 1.16 (m, 2H), 1.03 (m, 2H).

### EXAMPLE 124

### 1-cyclopropyl-8-methoxy-4-oxo-7-(4-((1,3-thiazol-2-ylcarbonyl)amino)-4,5,6,7-tetrahydro-1-benzothien-2-yl)-1,4-dihydro-3-quinolinecarboxylic acid

The desired product was prepared by substituting thiazole-2-carboxylic acid for acetic acid in Example 113. MS (DCI/NH₃) m/z 522 (M+H)⁺; ¹H NMR (300 MHz, DMSO-d₆) δ 9.01 (d, 1H), 8.80 (s, 1H), 7.96-8.08 (m, 4H), 7.60 (s, 1H), 5.18 (m, 1H), 4.24 (m, 1H), 3.67 (s, 3H), 2.83 (m, 2H), 1.80-2.15 (m, 4H), 1.17 (m, 2H), 1.02 (m, 2H).

### EXAMPLE 125

### 1-cyclopropyl-8-methoxy-7-(5-(methylamino)-4,5,6,7-tetrahydro-1-benzothien-2-yl)-4-oxo-1,4-dihydro-3-quinolinecarboxylic acid hydrochloride

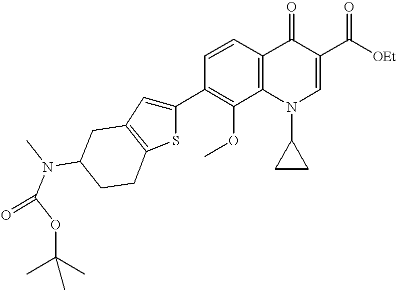

### EXAMPLE 125A

The desired product was prepared by substituting Example 76E for Example 40A in Example 77A.

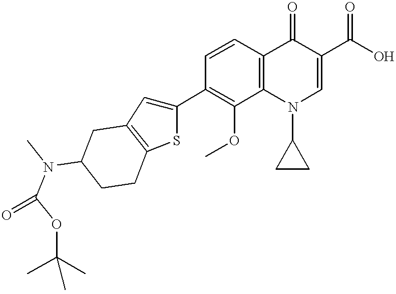

### EXAMPLE 125B

The desired product was prepared by substituting Example 125A for Example 2A in Example 2B.

### EXAMPLE 125C

### 1-cyclopropyl-8-methoxy-7-(5-(methylamino)-4,5,6,7-tetrahydro-1-benzothien-2-yl)-4-oxo-1,4-dihydro-3-quinolinecarboxylic acid hydrochloride

The desired product was prepared by substituting Example 125B for Example 40B in Example 40C. MS (DCI/NH3) m/z 425 (M+1)⁺; ¹H NMR (300 MHz, DMSO-d₆) δ 8.96 (s, 1H), 8.21 (d, 1H), 7.91(d, 1H), 7.53 (s, 1H), 4.29 (m, 1H) 3.70 (s, 3H), 3.59, (m, 1H), 3.28 (m, 1H), 3.05 (m, 2H), 2.83 (s, 3H), 2.77 (m, 1H), 2.40 (m, 1H), 2.14 (m, 1H), 1.31-1.03 (m, 4H).

### EXAMPLE 126

### 1-cyclopropyl-8-methoxy-7-(4-(4-morpholinylmethyl)-4,5,6,7-tetrahydro-1-benzothien-2-yl)-4-oxo-1,4-dihydro-3-quinolinecarboxylic acid

The desired product was prepared by substituting morpholine for pyrolidine in Example 111C. MS (DCI/NH₃) m/z 495 (M+H)⁺; ¹H-NMR (300 MHz, d₆-DMSO) δ 14.95 (br s, 1H), 8.78 (s, 1H), 8.11 (d, 1H), 7.97 (d, 1H), 7.86 (s, 1H), 4.25 (m, 1H), 3.65 (m, 7H), 2.96 (m, 1H), 2.76 (m, 2H), 2.60-2.38 (m, 6H), 1.95 (m, 4H), 1.15 (m, 2H), 1.03 (m, 2H).

### EXAMPLE 127

### 1-cyclopropyl-7-(4-((dimethylamino)methyl)-4,5,6,7-tetrahydro-1-benzothien-2-yl)-8-methoxy-4-oxo-1,4-dihydro-3-quinolinecarboxylic acid

The desired product was prepared by substituting N,N-dimethylamine for pyrolidine in Example 111C. MS (DCI/NH₃) m/z 453 (M+H)⁺; ¹H-NMR (300 MHz, d₆-DMSO) δ 8.73 (s, 1H), 8.07 (d, 1H), 7.92 (d, 1H), 7.72 (s, 1H), 4.20 (m, 1H), 3.65 (s, 3H), 2.87 (m, 1H), 2.75 (m, 2H), 2.47 (dd, 1H), 2.30 (dd, 1H), 2.21 (s, 6H), 1.90-1.60 (m, 4H), 1.13 (m, 2H), 0.99 (m, 2H).

### EXAMPLE 128

### 1-cyclopropyl-7-(4-(((dimethylamino)acetyl)amino)-4,5,6,7-tetrahydro-1-benzothien-2-yl)-8-methoxy-4-oxo-1,4-dihydro-3-quinolinecarboxylic acid

The desired product was prepared by substituting N,N-dimethylaminoacetic acid for acetic acid in Example 113. MS (DCI/NH₃) m/z 496 (M+H)⁺.

### EXAMPLE 129

### 1-cyclopropyl-8-methoxy-4-oxo-7-(4-((2-pyridinylacetyl)amino)-4,5,6,7-tetrahydro-1-benzothien-2-yl)-1,4-dihydro-3-quinolinecarboxylic acid

The desired product was prepared by substituting 2-pyridyl-acetic acid for acetic acid in Example 113. MS (DCI/NH₃) m/z 530 (M+H)⁺.

### EXAMPLE 130

### 7-(4-(aminomethyl)-4,5,6,7-tetrahydro-1-benzothien-2-yl)-1-cyclpopyl-8-methoxy-4-oxo-1,4-dihydro-3-quinolinecarboxylic acid

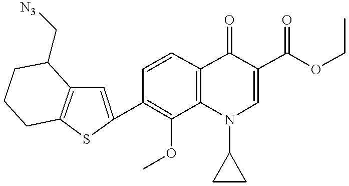

### EXAMPLE 130A

A solution of Example 111A (0.300 g, 0.564 mmol) and sodium azide (0.110 g, 1.695 mmol) in DMF (4 mL) was heated for 6 hours at 60°, cooled to room temperature and partitioned between ethyl acetate and water. The organic phase was washed with brine, dried (Na₂SO₄), and the crude product purified by silica gel chromatography eluting with 20% acetone in hexanes followed by 2% methanol in dichloromethane to give the desired product (0.232 g, 86% yield) as a yellow solid.

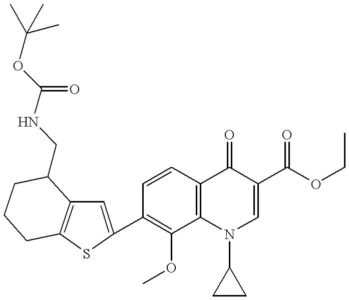

### EXAMPLE 130B

The desired product was prepared by substituting Example 130A for Example 201C in Example 201D and was purified by silica gel chromatography eluting with a gradient from 20-50% acetone in hexanes.

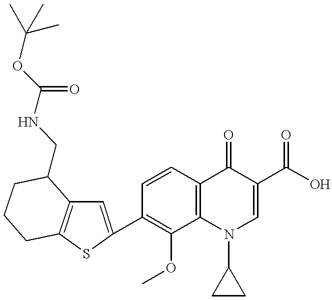

### EXAMPLE 130C

The desired product was prepared by substituting Example 130B for Example 2A in Example 2B.

### EXAMPLE 130D

### 7-(4-(aminomethyl)-4,5,6,7-tetrahydro-1-benzothien-2-yl)-1-cyclopropyl-8-methoxy-4-oxo-1,4-dihydro-3-quinolinecarboxylic acid

The desired product was prepared by substituting Example 130C for Example 59C in Example 59D. MS (DCI/NH₃) m/z 425 (M+H)⁺; ¹H-NMR (300 MHz, d₆-DMSO) δ 8.80 (s, 1H), 8.11 (d, 1H), 8.08 (br s, 2H), 8.05 (d, 1H), 7.80 (s, 1H), 4.25 (m, 1H), 3.68 (s, 3H), 3.10 (m, 1H), 2.92 (m, 1H), 2.80 (m, 1H), 1.95 (m, 2H), 1.80-1.60 (m, 2H), 1.15 (m, 2H), 1.03 (m, 2H).

### EXAMPLE 131

### 1-cyclopropyl-8-methoxy-4-oxo-7-((4E/Z)-4-((4-pyridinylmethoxy)imino)-4,5,6,7-tetrahydro-1-benzothien-2-yl)-1,4-dihydro-3-quinolinecarboxylic acid

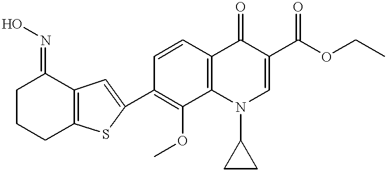

### EXAMPLE 131A

The desired product was obtained by substituting Example 48C for Example 50 in Example 61 A.

### EXAMPLE 131B

### 1-cyclopropyl-8-methoxy-4-oxo-7-((4E/Z)-4-((4-pyridiaylmethoxy)imino)-4,5,6,7-tetrahydro-1-benzothien-2-yl)-1,4-dihydro-3-quinolinecarboxylic acid

A solution of Example 131A (0.150 g, 0.33 nuol) in THF (6 ml) under positive N₂ atmosphere and was treated with Et₃N (183 μL, 1.32 mmol) and 4-picolylchloride hydrochloride (0.054 g, 0.33 mmol) and was heated at 50° C. for 12 hours. The reaction mixture was poured into 30 mL 10% NH₄Cl and extracted with CH₂Cl₂, dried (Na₂SO₄), filtered and concentrated to give the ethyl ester intermediate.

The desired product was obtained by substituting the ethyl ester intermediate above for Example 2A in Example 2B. mp 108-110° C.; MS (APCI) m/z 516 (M+H)⁺; ¹H NMR (300 MHz, DMSO-d₆) δ 8.80 (s, 1H), 8.55 (m, 2H), 8.10 (d, 1H), 8.05 (d, 1H), 7.82 (s, 1H), 7.40 (m, 2H), 5.25 (s, 2H), 4.25 (m, 1H), 3.68 (s, 3H), 3.30 (s, 1H), 2.90 (dd, 2H), 2.80 (dd, 2H), 1.98 (dd, 2H), 1.20 (m, 2H), 1.07 (m, 2H).

### EXAMPLE 132

### 7-(4-((2-aminoethyl)amino)-4,5,6,7-tetrahydro-1-benzothien-2-yl)-1-cyclopropyl-8-methoxy-4-oxo-1,4-dihydro-3-quinolinecarboxylic acid dihydrochloride

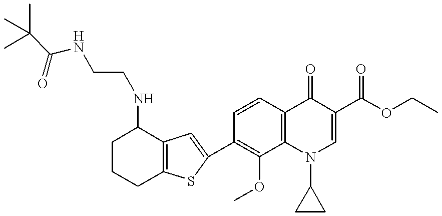

### EXAMPLE 132A

The desired product was prepared by substituting tert-butyl N-(2-oxoethyl)carbamate for 3-pyridine carboxaldehyde in Example 94A.

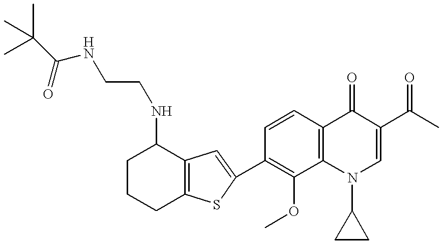

### EXAMPLE 132B

The desired product was prepared by substituting Example 132A for Example 2A in Example 2B.

### EXAMPLE 132C

### 7-(4-((2-aminoethyl)amino)-4,5,6,7-tetrahydro-1-benzothien-2-yl)-1-cyclopropyl-8-methoxy-4-oxo-1,4-dihydro-3-quinolinecarboxylic acid dihydrochloride

The desired product was prepared by substituting Example 132B for Example 40B in Example 40C. MS (DCI/NH₃) m/z 454 (M+H)⁺; ¹H NMR (300 MHz, CD₃OD) δ 8.97 (s, 1H), 8.23 (m, 1H0, 8.02 (m, 2H0, 4.65 (m, 1H), 4.28 (m, 1H), 3.73 (s, 3H), 3.56 (m, 2H), 3.42 (m, 2H), 3.00 (m, 2H), 2.02-2.28 (m, 4H), 1.28 (m, 2H), 1.07 (m, 2H).

### EXAMPLE 133

### 1-cyclopropyl-8-methoxy-7-(4-((1-methyl-4-piperidinyl)amino)-4,5,6,7-tetrahydro-1-benzothien-2-yl)-4-oxo-1,4-dihydro-3-quinolinecarboxylic acid dihydrochloride

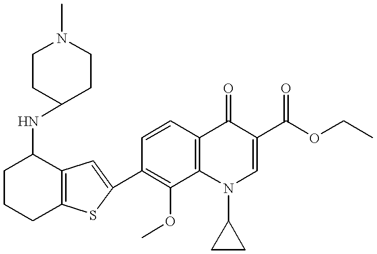

### EXAMPLE 133A

The desired product was prepared by substituting 1-methyl-4-piperidone and a reaction time of 24 hours for 3-pyridine carboxaldehyde and a reaction time of 2 hours in Example 94A.

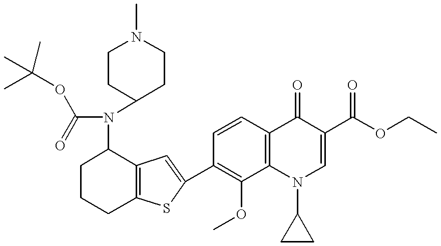

### EXAMPLE 133B

A solution of Example 133A (0.18 g, 0.35 mmol) in acetonitrile (12 mL) was treated with di-tert-butyl dicarbonate and a catalytic amount of dimethylaminopyridine at room temperature for 20 hours. The reaction mixture was partitioned between water and dichloromethane, dried (Na₂SO₄), filtered, concentrated and purified by silica gel chromatography eluting with 1% methanol in dichloromethane to yield 0.15 g (71%) of the desired compound.

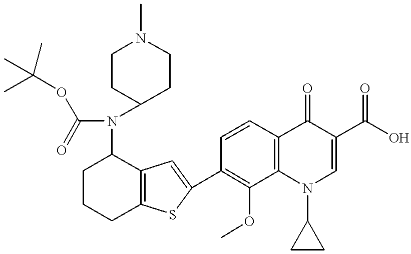

### EXAMPLE 133C The desired product was prepared by substituting Example 133B for Example 2A in Example 2B.

### EXAMPLE 133D

### 1-cyclopropyl-8-methoxy-7-(4-((1-methyl-4-piperidinyl)amino)-4,5,6,7-tetrahydro-1-benzothien-2-yl)-4-oxo-1,4-dihydro-3-quinolinecarboxylic acid dihydrochloride

The desired product was prepared by substituting Example 133C for Example 40B in Example 40C. MS (DCI/NH₃) m/z 508 (M+H)⁺; ¹H NMR (300 MHz, CD₃OD) δ 8.96 (s, 1H0, 8.22 (m, 1H), 8.03 (m, 2H), 4.76 (m, 1H), 4.29 (m, 1H), 3.83 (m, 1H), 3.73 (s, 3H), 3.66 (m, 1H), 3.83 (m, 1H), 3.73 (s, 3H), 3.66 (m, 1H), 3.25 (m, 2H), 3.00 (m, 4H), 2.93 (s, 3H), 2.02-2.58 (m, 6H), 1.28 (m, 2H), 1.08 (m, 2H).

### EXAMPLE 134

### 1-cyclopropyl-7-((4E/Z)-4-(hydroxyimino)-5-methyl-4,5,6,7-tetrahydro-1-benzothien-2-yl-8-methoxy-4-oxo-1,4-dihydro-3-quinolinecarboxylic acid

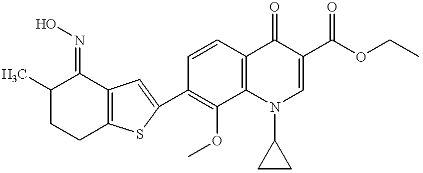

### EXAMPLE 134A

The desired product was prepared by substituting Example 106A and hydroxylamine hydrochloride for Example 50 and methoxylamine hydrochloride in Example 64.

### EXAMPLE 134B

### 1-cyclopropyl-7-((4E/Z)-4-(hydroxyimino)-5-methyl-4,5,6,7-tetrahydro-1-benzothien-2-yl-8-methoxy-4-oxo-1,4-dihydro-3-quinolinecarboxylic acid

The desired product was prepared by substituting Example 134A for Example 2A in Example 2B. mp 239-240° C.; MS (APCI) m/z 439 (M+H)⁺; ¹H NMR (300 MHz, DMSO-d₆) δ 8.82 (s, 0.15H), 8.80 (s, 0.85H), 8.20 (d, 0.15H), 8.13 (d, 0.85H), 8.00 (d, 0.85H) 7.95 (d, 0.15H), 7.89 (s, 1H), 4.25 (m, 1H), 3.72 (s, 0.45H), 3.70 (s, 2.55H), 3.50 (m, 1H), 3.40 (s, 1H), 3.00 (d, 2H), 2.70 (m, 2H), 1.90 (m, 1H), 1.25 (m, 2H), 1.15 (d, 3H), 1.07 (m, 2H).

### EXAMPLE 135

### 1-cyclopropyl-7-(4-hydroxy-7,7-dioxido-5,6-dihydro-4H-thieno[2,3-b]thiopyran-2-yl)-8-methoxy-4-oxo-1,4-dihydro-3-quinolinecarboxvlic acid

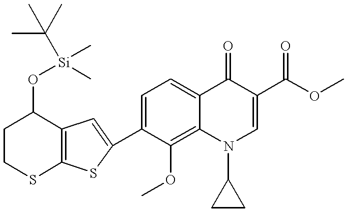

### EXAMPLE 135A

A solution of Example 36C (1.00 g, 1.75 mmol) and ethyl 7-bromo-1-cyclopropyl-8-methoxy-4-oxo-1,4-dihydro-3-quinolinecarboxylate (0.581 g, 1.58 mmol) in toluene (30 ml) was heated to 85° C. for 2 hour, allowed to cool to room temperature and was concentrated. The resulting brown oil was dissolved in MeOH (20 ml), cooled to 0° C. and treated with trimethylsilyldiazomethane (2.61 ml of a 2M solution in hexanes, 5.22 mmol). The reaction mixture was allowed to warm to room temperature, stir for 72 hours and concentrated. The crude residue was purified by silica gel chromatography eluting with 15% acetone in hexanes to give the desired product (0.530 g, 68%) as a yellow foam.

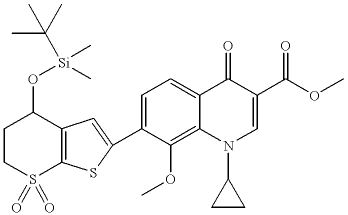

### EXAMPLE 135B

A solution of Example 135B (0.480 g, 0.72 mmol) in CH₂Cl₂ (15 ml) was cooled to 0° C. and treated portionwise with m-chloroperbenzoic acid (0.355 g, 1.43 mmol) and stirred for 3 hours. The reaction mixture was partitioned between saturated NaHCO₃ and CH₂Cl₂, the organic layer washed with 10% Na₂S₂O₃, saturated NaHCO₃, brine and dried (MgSO₄). The crude residue was purified by silica gel chromatography eluting with 35% acetone in hexanes to give the desired product (0.275 g, 65%) as a yellow foam.

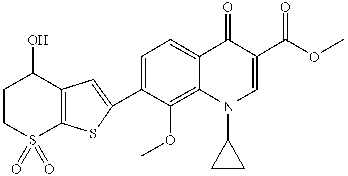

### EXAMPLE 135C

The desired product was prepared by substituting Example 135B for Example 35D in Example 35E and the crude residue purified by silica gel chromatography eluting with 3% MeOH in CH₂Cl₂.

### EXAMPLE 135D

### 1-cyclopropyl-7-(4-hydroxy-7,7-dioxido-5,6-dihydro-4H-thieno[2,3-b]thiopyran-2-yl)-8-methoxy-4-oxo-1,4-dihydro-3-quinolinecarboxylic acid

The desired product was prepared by substituting Example 135C for Example 48C in Example 48D to give the desired product. MS (APCI) m/z 496 (M+C1)⁻; ¹H NMR (300 MHz, DMSO-d₆) δ 8.83 (s, 1H), 8.19 (d, 1H), 8.14 (d, 1H), 7.92 (s, 1H), 5.92 (d, 1H), 4.87 (m, 1H), 4.27 (m, 1H), 3.75 (s, 3H), 3.67 (m, 2H), 2.54 (m, 1H), 2.37 (m, 1H), 1.19 (m, 2H), 1.04 (m, 2H).

### EXAMPLE 136

### 7-(4-(((5-chloro-1,3-dimethyl-1H-pyrazol-4-yl)sulfonyl)amino)-4,5,6,7-tetrahydro-1-benzothien-2-yl)-1-cyclopropyl-8-methoxy-4-oxo-1,4-dihydro-3-quinolinecarboxylic acid, bis(trifluoroacetic acid salt))

The Argonaut Quest 210 synthesizer was equipped with 5 ml reaction tubes. One tube was charged with PS-DIEA resin (Argonaut, 22-165 mesh, 1% DVB, 3.75mmol/g loading) (0.060 g, 0.225 mmol). A solution of Example 83A (0.050 g, 0.114 mmol) and 4-(dimethylamino)pyridine (0.010 g, 0.08 mmol) in 3 ml of 5:1/dichloromethane:dimethylacetamide was added. Then 5-chloro-1,3-dimethyl-1H-pyrazole-4-sulfonyl chloride (0.031 g, 0.137 mmol) was added and the mixture was shaken at room temperature overnight. PS-Trisamine resin (Argonaut, 100-200 mesh, 1% DVB, 4.06 mmol/g loading) (0.135 g, 0.55 mmol) was added and the mixture was shaken for 1 h. The mixture was filtered through the Quest reaction tube and the residual resin was washed with dichloromethane (2×2 ml). The combined filtrates were concentrated to dryness. The residue was dissolved in 3 ml of 1:1/tetrahydrofuran:methanol, 2N aqueous sodium hydroxide solution (1 ml, 2.0 mmol) was added and the mixture was shaken overnight. The mixture was neutralized with 4M hydrochloric acid in dioxane and concentrated to dryness. The residue was suspended in 1:1/dichloromethane:methanol (2 ml) and filtered to remove any inorganics. The resulting clear solution was concentrated to dryness and was purified by preparative reverse phase HPLC (Waters Prep Nova-Pak HR C18 column, 25×100 mm, 6 μm, 60 Å, 0.01% trifluoroacetic acid in water:acetonitrile) to give the desired product (0.008 g, 12%) as an off-white solid. MS (DCI/NH₃) m/z 604 (M+H)⁺; ¹H NMR (300 MHz, DMSO-d₆) δ 8.79 (s, 1H), 8.34 (d, 1H), 8.16 (d, 1H), 7.78 (d, 1H), 7.26 (s, 1H), 4.33 (m, 1H), 4.25 (m, 2H), 3.80 (s, 3H), 3.66 (s, 3H), 2.75 (m, 2H), 2.35 (s, 3H), 1.62-2.02 (m, 4H), 1.18 (m, 2H), 1.05 (m, 2H).

### EXAMPLE 137

### 7-(4-(((4-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting 4-cyanophenylsulphonyl chloride for 5-chloro-1,3-dimethyl-1H-pyrazole-4-sulfonyl chloride in Example 136. MS (DCI/NH₃) m/z 576 (M+H)⁺; ¹H NMR (300 MHz, DMSO-d₆) δ 8.79 (s, 1H), 8.53 (d, 1H), 8.17 (t, 2H), 8.10 (d, 2H), 7.63 (d, 1H), 6.97 (s, 1H), 4.46 (m, 1H), 4.23 (m, 1H), 3.64 (s, 3H), 2.74 (m, 2H), 1.58-1.96 (m, 4H), 1.15 (m, 2H), 1.02 (m, 2H).

### EXAMPLE 138

### 1-cylopropyl-8-methoxy-4-oxo-7-(4-((pheaylsulfonylamino)-4,5,6,7-tethydro-1-benzothien-2-yl)-1,4-dihydro-3-quinolinecarboxylic acid

The desired product was prepared by substituting phenylsulphonyl chloride for 5-chloro-1,3-dimethyl-1H-pyrazole-4-sulfonyl chloride in Example 136. MS (DCI/NH₃) m/z 551 (M+H)⁺; ¹H NMR (300 MHz, DMSO-d₆) δ 8.79 (s, 1H), 8.20 (d, 1H), 8.13 (d, 1H), 7.95 (dd, 2H), 7.73 (m, 3H), 7.60 (d, 1H), 6.93 (s, 1H), 4.38 (m, 1H), 4.24 (m, 1H), 3.62 (s, 3H), 2.72 (m, 2H), 1.88 (m, 1H), 1.74 (m, 2H) 1.63 (m, 1H), 1.16 (m, 2H), 1.02 (m, 2H).

### EXAMPLE 139

### 7-(4-(((2-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting 2-cyanophenylsulphonyl chloride for 5-chloro-1.3-dimethyl-1H-pyrazole-4-sulfonyl chloride in Example 136. MS (DCI/NH₃) m/z 576 (M+H)⁺; ¹H NMR (300 MHz, DMSO-d₆) δ 8.80 (s, 1H), 8.74 (d, 1H), 8.13-8.24 (m, 2H), 7.86-7.99 (m, 2H), 7.66 (d, H), 4.52 (m, 1H), 4.35 (m, 1H), 3.64 (s, 3H), 2.74 (m, 2H), 2.44 (m, 2H), 2.28 (m, 1H), 1.74 (m, 1H), 1.16 (m, 2H), 1.04 (m, 2H).

### EXAMPLE 140

### 1-cyclopropyl-8-methoxy-7-(4-(((4-methoxyphenyl)sulfoyl)amino)-4,5,6,7-tetrahydro-1-benzothien-2-yl)-4-oxo-1,4-dihydro-3-quinolinecarboxylic acid

The desired product was prepared by substituting 4-methoxyphenylsulphonyl chloride for 5-chloro-1.3-dimethyl-1H-pyrazole-4-sulfonyl chloride in Example 136. MS (DCI/NH₃) m/z 581 (M+H)⁺; ¹H NMR (300 MHz, DMSO-d₆) δ 8.78 (s, 1H), 8.10 (d, 1H), 8.03 (d, 1H), 7.86 (d, 1H), 7.60 (d, 1H), 7.19 (d, 1H), 6.93 (s, 1H), 4.33 (m, 1H), 4.24 (m, 1H), 3.86(s, 3H), 3.63 (s, 3H), 2.72 (m, 2H), 1.62-1.93 (m, 4H), 1.14 (m, 2H), 1.02 (m, 2H).

### EXAMPLE 141

### 1-cyclopropyl-8-methoxy-7-(4-(((3-nitrophenyl)sulfonyl)amino)-4,5,6,7-tetrahydro-1-benzothien-2-yl)-4-oxo-1,4-dihydro-3-quinolinecarboxylic acid

The desired product was prepared by substituting 3-nitrophenylsulphonyl chloride for 5-chloro-1.3-dimethyl-1H-pyrazole-4-sulfonyl chloride in Example 136. MS (DCI/NH₃) m/z 596 (M+H)⁺; ¹H NMR (300 MHz, DMSO-d₆) δ 8.79 (s, 1H), 8.65 (t, 1H), 8.56 (d, 1H), 8.54 (m, 1H), 8.36(m, 1H), 8.11 (d, 1H), 7.97 (t, 1H), 7.65 (d, 1H), 7.09 (s, 1H), 4.47 (m, 1H), 4.23 (m, 1H), 3.63 (s, 3H), 2.74 (m, 2H), 1.60-1.93 (m, 4H), 1.15 (m, 2H), 1.03 (m, 2H).

### EXAMPLE 142

### 1-cylopropyl-8-methoxy-4-oxo-7-(5-((2-pyrrolidinylmethyl)amino)-4,5,6,7-tetrahydro-1-benzothien-2-yl)-1,4-dihydro-3-quinolinecarboxylic acid dihydrochloride

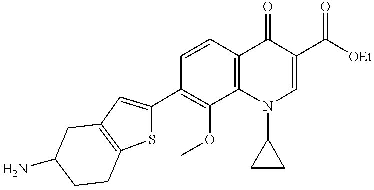

### EXAMPLE 142A

The desired product was prepared by substituting Example 76E for Example 40A in Example 83A.

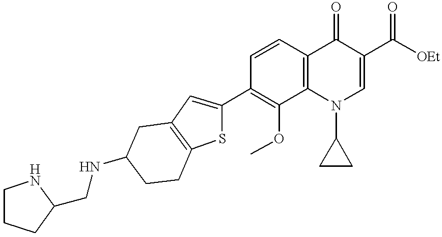

### EXAMPLE 142B

The desired product was prepared by substituting Example 142A and N-(tert-butoxycarbonyl)-prolinal for Example 83A and 3-pyridine carboxaldehyde in Example 94A.

### EXAMPLE 142C

### 1-cyclopropyl-8-methoxy-4-oxo-7-(5-((2-pyrrolidinylmethyl)amino)-4,5,6,7-tetrahydro-1-benzothien-2-yl)-1,4-dihydro-3-quinolinecarboxylic acid dihydrochloride

The desired product was prepared by substituting Example 142B for Example 2A in Example 2B followed by treatment with 4.0 N HCl in dioxane. MS (APCI/NH3) m/z 494 (M+1)⁺; ¹H NMR (300 MHz, DMSO-d₆) δ 8.80 (s, 1H), 8.13 (d, 1H), 8.01 (s, 1H), 7.64 (s, 1H), 4.25 (m, 1H), 3.93 (m, 1H), 3.69 (s, 3H), 3.45 (m, 2H), 3.10-2.80 (m, 3H,) 2.40-1.70 (m, 8H), 1.20-1.00 (m, 4H).

### EXAMPLE 143

### 7-(4-amino-7,7-dioxido-5,6-dihydro-4H-thieno[2,3-b]thiopyran-2-yl)-1-cyclopropyl-8-methoxy-4-oxo-1,4-dihydro-3-quinolinecarboxylic acid hydrochloride

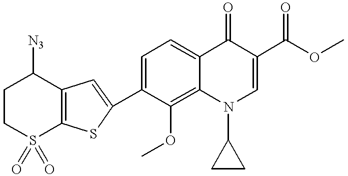

### EXAMPLE 143A

The desired product was prepared by substituting Example 135C for Example 37A in Example 37B and the crude residue purified by silica gel chromatography eluting with 50% acetone in hexanes.

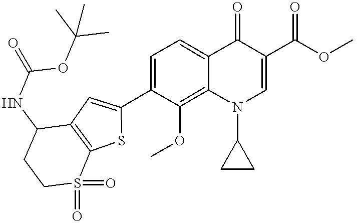

### EXAMPLE 143B

The desired product was prepared by substituting Example 143A for Example 201C in Example 201C and the crude residue purified by silica gel chromatography eluting with 40% acetone in hexanes.

### EXAMPLE 143C

### 7-(4-amino-7,7-dioxido-5,6-dihydro-4H-thieno[2,3-b]thiopyran-2-yl)-1-cyclopropyl-8-methoxy-4-oxo-1,4-dihydro-3-quinolinecarboxylic acid hydrochloride

The desired product was prepared by substituting Example 143B for Example 48C in Example 48D. MS (DCI/NH₃) m/z 461 (M+H)⁺; ¹H NMR (300 MHz, DMSO-d₆) δ 8.85 (s, 1H), 8.28 (d, 1H), 8.26 (d, 1H), 7.98 (s, 1H), 4.78 (m, 1H), 4.28 (m, 1H), 3.86 (m, 1H), 3.77 (s, 3H), 2.78 (m, 1H), 2.63 (m, 1H), 1.21 (m, 2H), 1.06 (m, 2H).

### EXAMPLE 144

### 1-cyclopropyl-7-(4-(((3,5-dimethyl-4-isoxazolyl)sulfonyl)amino)-4,5,6,7-tetrahydro-1-benzothien-2-yl)-8-methoxy-4-oxo-1,4-dihydro-3-quinolinecarboxylic acid, trifluoroacetic acid salt

The desired product was prepared by substituting 3,5-dimethylisoxazole-4-sulphonyl chloride for 5-chloro-1.3-dimethyl-1H-pyrazole-4-sulfonyl chloride in Example 136. MS (DCI/NH₃) m/z 570 (M+H)⁺; ¹H NMR (300 MHz, DMSO-d₆) δ 8.79 (s, 1H), 8.56 (d, 1H), 8.14 (d, 1H), 7.81 (d, 1H), 7.31 (s, 1H), 4.48 (m, 1H), 4.36 (m, 1H), 3.65 (s, 3H), 2.76 (m, 2H), 2.65 (s, 3H), 2.39 (s, 3H), 1.64-1.98 (m, 4H), 1.18 (m, 2H), 1.05 (m, 2H).

### EXAMPLE 145

### 7-(4-((2,1,3-benzoxadiazol-4-ylsulfonyl)amino)-4,5,6,7-tetrahydro-1-benzothien-2-yl)-1-cyclopropyl-8-methoxy-4-oxo-1,4-dihydro-3-quinolinecarboxylic acid, bis(trifluoroacetic acid salt)

The desired product was prepared by substituting 2,1,3-benzoxadiazole-4-sulphonyl chloride for 5-chloro-1,3-dimethyl-1H-pyrazole-4-sulfonyl chloride in Example 136. MS (DCI/NH₃) m/z 593 (M+H)⁺; ¹H NMR (300 MHz, DMSO-d₆) δ 8.86 (d, 1H), 8.78 (s, 1H), 8.44 (d, 1H), 8.20 (d, 1H), 8.11 (d, 1H), 7.81 (dd, 1H), 7.53 (d, 1H), 7.05 (s, 1H), 4.65 (m, 1H), 4.22 (m, 1H), 3.59 (s, 3H), 2.72(m, 2H), 1.63-1.98 (m, 4H), 1.15 (m, 2H), 1.03 (m, 2H).

### EXAMPLE 146

### 1-cyclopropyl-7-(4-(((dimethylamino)sulfonyl)amino)-4,5,6,7-tetrahydro-1-benzothien-2-yl)-8-methoxy-4-oxo-1,4-dihydro-3-quinolinecarboxylic acid

The desired product was prepared by substituting N,N-dimethylsulphamoyl chloride for 5-chloro-1,3-dimethyl-1H-pyrazole-4-sulfonyl chloride in Example 136. MS (DCI/NH₃) m/z 518 (M+H)⁺; ¹H NMR (300 MHz, DMSO-d₆) δ 8.79 (s, 1H), 8.15 (d, 1H), 7.92 (d, 1H), 7.70 (s, 1H), 7.63 (d, 1H), 4.38 (m, 1H), 4.27 (m, 1H), 3.69 (s, 3H), 2.75 (s, 6H), 2.02 (m, 2H), 1.84 (m, 2H), 1.16 (m, 2H), 1.04 (m, 2H).

### EXAMPLE 147

### 1-cyclopropyl-8-methoxy-4-oxo-7-(4-((2-thienylsulfonyl)amino)-4,5,6,7-tetrahydro-1-benzothien-2-yl)-1,4-dihydro-3-quinolinecarboxulic acid

The desired product was prepared by substituting thiophene-2-sulphonyl chloride for 5-chloro-1.3-dimethyl-1H-pyrazole-4-sulfonyl chloride in Example 136. MS (DCI/NH₃) m/z 557 (M+H)⁺; ¹H NMR (300 MHz, DMSO-d₆) δ 8.79 (s, 1H), 8.40 (d, 1H), 8.16 (d, 1H), 8.04 (dd, 1H), 7.75 (dd, 1H), 7.69 (d, 1H), 7.28 (dd, 1H), 6.97 (s, 1H), 4.44 (m, 1H), 4.24 (m, 1H), 3.64 (s, 3H), 2.74 (m, 2H), 1.03-1.96 (m, 4H), 1.16 (m, 2H), 1.04 (m, 2H).

### EXAMPLE 148

### 7-(4-(((3-canophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting 3-cyanophenylsulphonyl chloride for 5-chloro-1.3-dimethyl-1H-pyrazole-4-sulfonyl chloride in Example 136. MS (DCI/NH₃) m/z 576 (M+H)⁺; ¹H NMR (300 MHz, DMSO-d₆) δ 8.79 (s, 1H), 8.44 (d, 1H), 8.36 (m, 1H), 8.23 (m, 2H), 8.15 (d, 1H), 7.90 (t, 1H), 7.65 (d, 1H), 7.00 (s, 1H), 4.48 (m, 1H), 4.24 (m, 1H), 3.64 (s, 3H), 2.75 (m, 2H), 1.60-1.94 (m, 4H), 1.16 (m, 2H), 1.03 (m, 2H).

### EXAMPLE 149

### 7-(4-(((4-(acetylamino)phenyl)sulfonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting 4-acetamidobenzenesulphonyl chloride for 5-chloro-1.3-dimethyl-1H-pyrazole-4-sulfonyl chloride in Example 136. MS (DCI/NH₃) m/z 608 (M+H)⁺; ¹H NMR (300 MHz, DMSO-d₆) δ 10.43 (br s, 1H), 8.78 (s, 1H), 8.08 (t, 2H), 7.86 (s, 4H), 7.46 (d, 1H), 6.65 (s, 1H), 4.35 (m, 1H), 4.22 (m, 2H), 3.62 (s, 3H), 2.72 (m, 2H), 2.15 (s, 3H), 1.65-1.95 (m, 4H), 1.12 (m, 2H), 1.01 (m, 2H).

### EXAMPLE 150

### 7-(4-((2,1,3-benzothiadiazol-4-ylsulfonyl)amino)-4,5 6,7-tetrahydro-1-benzothien-2-yl)-1-cyclopropvl-8-methoxy-4-oxo-1 4-dihydro-3-quinolinecarboxylic acid bis(trifluoroacetic acid salt)

The desired product was prepared by substituting 2,1,3-benzothiadiazole-4-sulphonyl chloride for 5-chloro-1.3-dimethyl-1H-pyrazole-4-sulfonyl chloride in Example 136. MS (DCI/NH₃) m/z 609 (M+H)⁺; ¹H NMR (300 MHz, DMSO-d₆) δ 8.78 (s, 1H), 8.48 (dd, 2H), 8.32 (d, 1H), 8.11 (d, 1H), 7.92 (dd, 1H), 7.38 (d, 1H), 6.86 (s, 1H), 4.76 (m, 1H), 4.21 (m, 1H), 3.57 (s, 3H), 2.72 (m, 2H), 1.59-1.97 (m, 4H), 1.24 (m, 2H), 1.14 (m, 2H), 1.01 (m, 2H).

### EXAMPLE 151

### 1-cyclopropyl-7-(4-(((5-(3-isoxazolyl)-2-thienyl)sulfonyl)amino)-4,5,6,7-tetrahydro-1-benzothien-2-yl)-8-methoxy-4-oxo-1,4-dihydro-3-quinolinecarboxylic acid trifluoroacetic acid salt

The desired product was prepared by substituting 5-isoxazole-3-ylthiophene-2-sulphonyl chloride for 5-chloro-1.3-dimethyl-1H-pyrazole-4-sulfonyl chloride in Example 136. MS (DCI/NH₃) m/z 624 (M+H)⁺; ¹H NMR (300 MHz, DMSO-d₆) δ 8.79 (s, 1H), 8.14 (d, 1H), 8.03 (d, 1H), 7.86 (d, 1H), 7.78 (d, 1H), 7.29 (s, 1H), 4.78 (s, 1H), 4.52 (m, 1H), 4.24 (m, 1H), 3.66 (s, 3H), 2.74 (m, 2H), 1.62-1.98 (m, 4H), 1.15 (m, 2H), 1.02 (m, 2H).

### EXAMPLE 152

### 1-cyclopropyl-7-(4-(((4-fluorophenyl)sulfonyl)amino)-4,5,6,7-tetrahydro-1-benzothien-2-yl)-8-methoxU-4-oxo-1,4-dihydro-3-quinolinecarboxylic acid

The desired product was prepared by substituting 4-fluorophenylsulphonyl chloride for 5-chloro-1.3-dimethyl-1H-pyrazole-4-sulfonyl chloride in Example 136. MS (DCI/NH₃) m/z 569 (M+H)⁺; ¹H NMR (300 MHz, DMSO-d₆) δ 8.79 (s, 1H), 8.24 (d, 1H), 8.13 (d, 1H), 7.99 (dd, 2H), 7.65 (d, 1H), 7.55 (dd, 2H), 7.01 (s, 1H), 4.40 (m, 1H), 4.24 (m, 1H), 3.64 (s, 3H), 2.72 (m, 2H), 1.57-1.94 (m, 4H), 1.16 (m, 2H), 1.03 (m, 2H).

### EXAMPLE 153

### 7-(4-(((6-chloroimidazo[2,1-b][1,3]thiazol-5-yl)sulfonyl)amino)-4,5,6,7-tetrahydro-1-benzothien-2-yl)-1-cyclopropyl-8-methoxy-4-oxo-1,4-dihydro-3-quinolinecarboxylic acid bis(trifluoroacetic acid salt)

The desired product was prepared by substituting 6-chloroimidazo[2,1-b ]thiazole-5-sulphonyl chloride for 5-chloro-1.3-dimethyl-1H-pyrazole-4-sulfonyl chloride in Example 136. MS (DCI/NH₃) m/z 632 (M+H)⁺; ¹H NMR (300 MHz, DMSO-d₆) δ 8.96 (d, 1H), 8.80 (s, 1H), 8.14 (d, 1H), 8.03 (d, 1H), 7.69 (d, 1H), 7.56 (d, 1H), 6.90 (s, 1H), 4.45 (m, 1H), 4.23 (m, 1H), 3.62 (s, 3H), 2.76 (m, 2H), 1.68-1.96 (m, 4H), 1.17 (m, 2H), 1.03 (m, 2H).

### EXAMPLE 154

### 1-cyclopropyl-8-methoxy-4-oxo-7-(4-((4-pyridinylacetyl)amino)-4,5,6,7-tetrahydro-1-benzothien-2-yl)-1,4-dihydro-3-quinolinecarboxylic acid

The desired product was prepared by substituting 4-pyridyl-acetic acid for acetic acid in Example 113. MS (DCI/NH₃) m/z 530 (M+H)⁺.

### EXAMPLE 155

### 1-cyclopropyl-7-(4-((2-hydroxyethyl)amino)-4,5,6,7-tetrahydro-1-benzothien-2-yl)-8-methoxy-4-oxo- 1 4-dihydro-3-quinolinecarboxlic acid hydrochloride

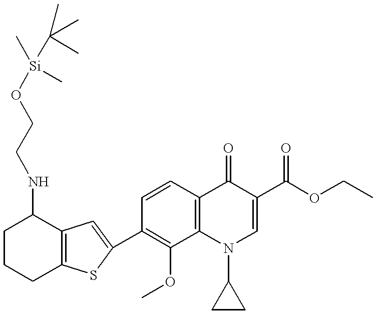

### EXAMPLE 155A

The desired product was prepared by substituting (tert-butyldimethylsilyloxy)-acetaldehyde and a reation temperature of 0° C. for 3-pyridine carboxaldehyde and an ambient reation temperature, respectively in Example 94A.

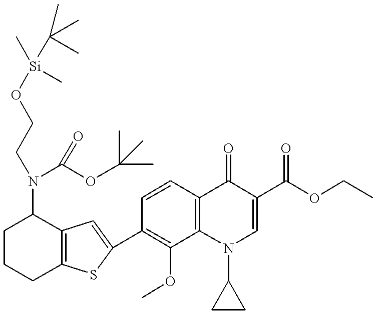

### EXAMPLE 155B

The desired product was prepared by substituting Example 155A for Example 133A in Example 133B.

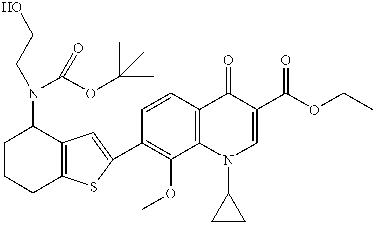

### EXAMPLE 155C

The desired product was prepared by substituting Example 155B for Example 35D in Example 35E.

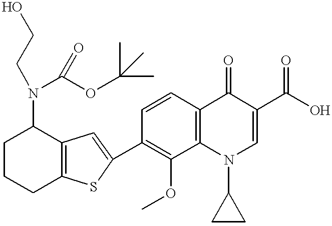

### EXAMPLE 155D

The desired product was prepared by substituting Example 155C for Example 2A in Example 2B.

### EXAMPLE 155E

### 1-cyclopropyl-7-(4-((2-hydroxyethyl)amino)-4,5,6,7-tetrahydro-1-benzothien-2-yl)-8-methoxy-4-oxo-1,4-dihydro-3-ouinolinecarboxylic acid hydrochloride

The desired product was prepared by substituting Example 155D for Example 40B in Example 40C. MS (DCI/NH₃) m/z 455 (M+H)⁺; 1H NMR (300 MHz, CD₃OD) δ 8.97 (s.1H0, 8.24 (d, 1H), 7.94 (m, 1H), 7.87 (m, 1H). 4.62 (m, 1H), 4.28 (m, 1H), 3.88 (m, 2H), 3.72 (s, 3H), 3.26 (m, 2H), 2.99 (m, 2H), 1.94-2.27 (m, 4H), 1.28 (m, 2H), 1.08 (m, 2H).

### EXAMPLE 156

### 7-(4-amino-4,5,6,7-tetrahydro-1-benzothien-2-yl)-1-cyclopropyl-6-fluoro-4-oxo-1,4-dihydro[1,8]naphthyridine-3-carboxylic acid hydrochloride

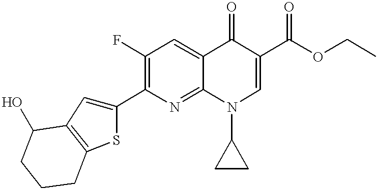

### EXAMPLE 156A

The desired product was prepared by substituting Example 35C for Example 35D in Example 35E and was used without further purification.

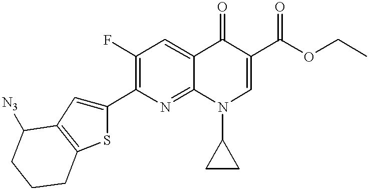

### EXAMPLE 156B

The desired product was prepared by substituting Example for Example 37A in Example 37B and the crude product purified by silica gel chromatography eluting with 50% ethyl acetate in hexanes.

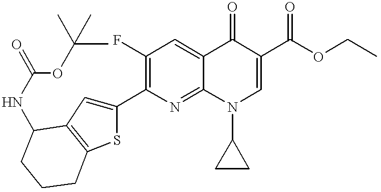

### EXAMPLE 156C

The desired product was prepared by substituting Example 156B for 201C in Example 201C and the crude product recrystalized from ethyl acetate.

### EXAMPLE 156D

### 7-(4-amino-4,5,6,7-tetrahydro-1-benzothien-2-yl)-1-cyclopropyl-6-fluoro-4-oxo-1,4-dihydro[1,8]naphthyridine-3-carboxylic acid hydrochloride

A solution of Example 156C (0.134 g, 0.247 mmol) in THF (10 ml) was treated with 1 N HCl (10 ml) and heated to 80° C. for 14 hours. The resulting heterogeneous mixture was filtered and the white solid obtained suspended in CH2Cl2 (5 ml) and treated with HCl (5 ml of a 4N solution in dioxane). After stirring for 4 hours, the mixture was filtered to give the desired product (0.079 g, 80%) as a yellow solid. MS (APCI) m/z 383 (M-NH₂)⁺; ¹H NMR (300 MHz, DMSO-d₆) δ 8.82 (s, 1H), 8.57 (d, 1H), 8.51 (br s, 3H), 8.28 (d, 1H), 4.46 (m, 1H), 3.83 (m, 1H), 2.89 (m, 2H), 2.20-1.97 (m, 2H), 1.93-1.77 (m, 2H), 1.31 (m, 2H), 1.18 (m, 2H).

### EXAMPLE 157

### 7-(4-((glycyl)amino)-4,5,6,7-tetrahydro-1-benzothien-2-yl)-1-cyclopropyl-8-methoxy-4-oxo-1,4-dihydro-3-quinolinecarboxylic acid trifluoroacetic acid salt

The Argonaut Quest 210 synthesizer was equipped with 5 ml reaction tubes. One tube was charged with 1 ml 1:1/dichloromethane:dimethylacetamide, N-cyclohexylcarbodiimide, N′-methyl polystyrene HL (NovaBiochem, 200-400 mesh, 2% DVB, 1.52 mmol/g loading) (0.151 g, 0.23 mmol), 1-hydroxybenzotriazole hydrate (0.003 g, 0.019 mmol) and N-(9-fluorenylmethoxycarbonyl)-glycine (0.051 g, 0.171 mmol). The mixture was stirred for 30 min before a solution of Example 83A (0.05 g, 0.114 mmol) in 1 ml of 1:1 / dichloromethane:dimethylacetamide was added. The mixture was stirred 48 h at room temperature. PS-Trisamine resin (Argonaut, 100-200 mesh, 1% DVB, 4.06 mmol/g loading) (0.135 g, 0.55 mmol) was added and the mixture was agitated for 2 h. Piperidinomethyl polystyrene HL resin (NovaBiochem, 200-400 mesh, 2% DVB, 3.5 mmol/g loading) (0.244 g, 0.855 mmol) was added and the mixture was shaken for 7 days. The mixture was filtered through the Quest reaction tube and the residual resin was washed with dichloromethane (2×2 ml). The filtrate was concentrated to dryness and the residue was redissolved in 3 ml of 1:1/tetrahydrofuran:methanol. 2N aqueous sodium hydroxide solution (1 ml) was added and the mixture was shaken overnight. The mixture was neutralized with 4M hydrochloric acid in dioxane and concentrated to dryness. The residue was suspended in 1:1/dichloromethane:methanol (2 ml) and was filtered to remove any inorganics. The resulting clear solution was concentrated to dryness and was purified by preparative reverse phase HPLC (Waters Prep Nova-Pak HR C18 column, 25×100 mm, 6 μm, 60 Å, 0.01% trifluoroacetic acid in water:acetonitrile) to give the desired product (0.005 g, 12%) as an off-white solid. MS (DCI/NH₃) m/z 468 (M+H)⁺; ¹H NMR (300 MHz, DMSO-d₆) δ 14.97 (br s, 1H), 8.80 (s, 1H), 8.72 (d, 1H), 8.12 (d, 1H), 7.96 (d, 1H), 7.78 (d, 1H), 7.58 (s, 1H), 5.01 (m, 2H), 4.26 (m, 1H), 3.70 (s, 3H), 2.84 (m, 2H), 1.98 (m, 2H), 1.79 (m, 1H), 1.71 (m, 1H), 1.18 (m, 2H), 1.04 (m, 2H).

### EXAMPLE 158

### 7-(4-((D-alanyl)amino)-4,5,6,7-tetrahydro-1-benzothien-2-yl)-1-cyclopropyl-8-methoxy-4-oxo-1,4-dihydro-3-quinolinecarboxylic acid trifluoroacetic acid salt

The desired product was prepared by substituting N-(9-fluorenylmethoxycarbonyl)-L-alanine for N-(9-fluorenylmethoxycarbonyl)-glycine in Example 157. MS (DCI/NH₃) m/z 482 (M+H)⁺; ¹H NMR (300 MHz, DMSO-d₆) δ 8.79 (s, 1H), 8.75 (dd, 1H), 8.12 (d, 1H), 7.93 (dd, 1H), 7.52 (d, 1H), 4.98 (m, 1H), 4.26 (m, 1H), 3.84 (m, 1H), 3.70 (d, 3H), 2.83 (m, 2H), 1.98 (m, 2H), 1.88 (m, 1H), 1.72 (m, 1H), 1.40 (dd, 3H), 1.18 (m, 2H), 1.04 (m, 2H).

### EXAMPLE 159

### 1-cyclopropyl-8-methoxy-4-oxo-7-(4-((D-prolyl)amino)-4,5,6,7-tetrahydro-1-benzothien-2-yl)-1,4-dihydro-3-quinolinecarboxylic acid, trifluoroacetic acid salt

The desired product was prepared by substituting N-(9-fluorenylmethoxycarbonyl)-L-proline for N-(9-fluorenylmethoxycarbonyl)-glycine in Example 157. MS (DCI/NH₃) m/z 508 (M+H)⁺; ¹H NMR (300 MHz, DMSO-d₆) δ 9.70 (br s, 1H), 9.51 (br s, 1H), 8.91 (dd, 1H), 8.79 (s, 1H), 7.95 (dd, 1H), 7.59 (d, 1H), 5.0 (m, 1H), 4.26 (m, 1H), 4.20 (m, 1H), 3.70 (d, 3H), 3.60 (m, 2H), 2.84 (m, 2H), 2.32 (m, 1H), 1.95 (m, 5H), 1.88 (m, 1H), 1.73 (m, 1H), 1.18 (m, 2H), 1.04 (m, 2H).

### EXAMPLE 160

### 7-(4-(((2R)-2-amino-3-(1H-imidazol-5-yl)propanoyl)amino)-4,5,6,7-tetrahydro-1-benzothien-2-yl)-1-cyclopropyl-8-methoxv-4-oxo-1,4-dihydro-3-quinolinecarboxylic acid bis(trifluoroacetic acid salt)

The desired product was prepared by substituting N-α-(9-fluorenylmethoxycarbonyl)-N-trityl-L-histidine for N-(9-fluorenylmethoxycarbonyl)-glycine in Example 157. MS (DCI/NH₃) m/z 548 (M+H)⁺; ¹H NMR (300 MHz, DMSO-d₆) δ 14.90 (br s, 1H), 8.96 (d, 1H), 8.81 (dt, 1H), 8.79 (d, 1H), 8.15 (dd, 1H), 7.90 (dd, 1H), 7.47 (dd, 1H), 4.95 (m, 1H), 4.25 (m, 1H), 4.11 (m, 2H), 3.68 (d, 3H), 3.22 (m, 2H), 2.80 (m, 2H), 1.97 (m, 1H), 1.86 (m, 1H), 1.80 (m, 2H), 1.50 (m, 1H), 1.17 (m, 2H), 1.03 (m, 2H).

### EXAMPLE 161

### 7-(4-((leucyl)amino)-4,5,6,7-tetrahydro-1-benzothien-2-yl)-1-cyclopropyl-8-methoxy-4-oxo-1,4-dihydro-3-quinolinecarboxylic acid, trifluoroacetic acid salt

The desired product was prepared by substituting N-(9-fluorenylmethoxycarbonyl)-D-leucine for N-(9-fluorenylmethoxycarbonyl)-glycine in Example 157. MS (DCI/NH₃) m/z 524 (M+H)⁺; ¹H NMR (300 MHz, DMSO-d₆) δ 8.86 (dd, 1H), 8.80 (d, 1H), 8.14 (dd, 1H), 7.90 (dd, 1H), 7.53 (d, 1H), 4.99 (m, 1H), 4.25 (m, 1H), 3.68 (d, 3H), 2.84 (m, 2H), 2.72 (m, 1H), 1.55-2.04 (m, 7H), 1.16 (m, 2H), 1.04 (m, 2H).

### EXAMPLE 162

### 7-(4-((D-tyrosyl)amino)-4,5,6,7-tetrahydro-1-benzothien-2-yl)-1-cyclopropyl-8-methoxy-4-oxo-1,4-dihydro-3-quinolinecarboxylic acid trifluoroacetate

The desired product was prepared by substituting N-(9-fluorenylmethoxycarbonyl)-4-(diethylphosphono)-L-tyrosine for N-(9-fluorenylmethoxycarbonyl)-glycine in Example 157. MS (DCI/NH₃) m/z 574 (M+H)⁺; ¹H NMR (300 MHz, DMSO-d₆) δ 14.89 (br s, 1H), 8.79 (d, 1H), 8.68 (dd, 1H), 8.30 (m, 1H), 8.25 (m, 1H), 7.00-7.24 (m, 4H), 6.72 (d, 1H0, 4.90 (m, 1H), 4.24 (m, 1H), 3.95 (m, 1H), 3.90 (m, 1H), 3.67 (d, 3H0, 3.00 (m, 1H), 2.75 (m, 2H), 1.92 (m, 1H), 1.86 (m, 1H), 1.75 (m, 2H), 1.44 (m, 1H), 1.16 (m, 2H), 1.05 (m, 2H).

### EXAMPLE 163

### 7-(4-((O-methyl-D-tyrosyl)amino)-4,5,6,7-tetrahydro-1-benzothien-2-yl)-1-cyclopropyl-8-methoxy-4-oxo-1,4-dihydro-3-quinolinecarboxylic acid, trifluoroacetic acid salt

The desired product was prepared by substituting N-(9-fluorenylmethoxycarbonyl)-O-methyl-L-tyrosine for N-(9-fluorenylmethoxycarbonyl)-glycine in Example 157. MS (DCI/NH₃) m/z 588 (M+H)⁺; ¹H NMR (300 MHz, DMSO-d₆) δ 8.79 (d, 1H), 8.66 (dt, 1H), 8.13 (dd, 1H), 7.85 (dd, 1H), 7.17 (dt, 2H), 6.87 (dd, 2H), 4.91 (m, 1H), 4.26 (m, 1H), 3.78 (s, 3H), 3.68 (d, 3H), 3.52 (s, 2H), 3.00 (m, 2H), 2.78 (m, 2H), 1.66-2.00 (m, 4H), 1.17 (m, 2H), 1.02 (m, 2H).

### EXAMPLE 164

### 7-(4-((D-methionyl)amino)-4,5,6,7-tetrahydro-1-benzothien-2-yl)-1-cyclopropyl-8-methoxy-4-oxo-1 4-dihydro-3-guinolinecarboxvlic acid trifluoroacetate

The desired product was prepared by substituting N-(9-fluorenylmethoxycarbonyl)-L-methionine for N-(9-fluorenylmethoxycarbonyl)-glycine in Example 157. MS (DCI/NH₃) m/z 542 (M+H)⁺; ¹H NMR (300 MHz, DMSO-d₆) δ 8.86 (d, 1H), 8.79 (s, 1H), 8.10 (d, 1H), 7.88 (d, 1H), 7.54 (s, 1H), 5.00 (m, 1H), 4.26 (m, 1H), 3.70-3.89 (m, 4H), 3.69 (d, 3H), 2.82 (m, 2H), 2.46-2.50 (m, 3H), 1.94-2.10 (m, 3H), 1.89 (m, 1H), 1.79 (m, 1H), 1.17 (m, 2H), 1.03 (m, 2H).

### EXAMPLE 165

### 7-(4-(((2R)-2-amino-3-(3-pyridinyl)propanoyl)amino)-4,5,6,7-tetrahydro-1-benzothien-2-yl)-1-cyclopropyl-8-methoxy-4-oxo-1,4-dihydro-3-quinolinecarboxylic acid bis(trifluoroacetic acid salt)

The desired product was prepared by substituting N-(9-fluorenylmethoxycarbonyl)-L-3-pyridylalanine for N-(9-fluorenylmethoxycarbonyl)-glycine in Example 157. MS (DCI/NH₃) m/z 558 (M+H)⁺; ¹H NMR (300 MHz, DMSO-d₆) δ 8.80 (dt, 1H), 8.59 (dt, 2H), 8.50 (d, 2H), 8.16 (dd, 1H), 7.90 (dd, 1H), 7.55 (m, 1H), 4.90 (m, 1H), 4.25 (m, 1H), 3.68 (d, 3H), 3.15 (m, 2H), 2.78 (m, 2H), 2.52 (s, 2H), 2.50 (m, 1H), 1.66-2.00 (m, 4H), 1.40 (m, 1H), 1.17 (m, 2H), 1.02 (m, 2H).

### EXAMPLE 166

### 1-cyclopropl-8-methoxy-4-oxo-7-(4-(((2R)-piperidinylcarbonyl)amino)-4,5,6,7-tetrahydro-1-benzothien-2-yl)-1,4-dihydro-3-quinolinecarboxylic acid, trifluoroacetic acid salt

The desired product was prepared by substituting N-(9-fluorenylmethoxycarbonyl)-L-pipecholinic acid for N-(9-fluorenylmethoxycarbonyl)-glycine in Example 157. MS (DCI/NH₃) m/z 522 (M+H)⁺; ¹H NMR (300 MHz, DMSO-₆) δ 9.19 (dd, 1H), 8.82 (dd, 1H), 8.72 (m, 1H), 8.11 (dd, 1H), 7.93 (dd, 1H), 7.52 (d, 1H), 4.99 (m, 1H), 4.26 (m, 1H), 4.10 (m, 2H), 3.78 (m, 1H), 3.68 (d, 3H), 3.29 (m, 1H), 2.96 (m, 1H), 2.82 (m, 2H), 2.12 (m, 1H), 1.98 (m, 2H), 1.42-1.81 (m, 4H), 1.18 (m, 2H), 1.03 (m, 2H).

### EXAMPLE 167

### 1-cyclopropyl-8-methoxy-4-oxo-7-(4-((4-pyrimidinylcarbonyl)amino)-4,5,6,7-tetrahydro-1-benzothien-2-yl -1,4-dihydro-3-quinolinecarboxylic acid bis(trifluoroacetic acid salt)

The desired product was prepared by substituting pyrimidine-4-carboxylic acid for acetic acid in Example 113. MS (DCI/NH₃) m/z 517 (M+H)⁺; ¹H NMR (300 MHz, DMSO-d₆) δ 14.90 (br s, 1H), 9.31 (d, 1H), 9.08 (d, 2H), 8.78 (s, 1H), 8.10 (dd, 1H), 8.02 (dd, 2H), 7.60 (s, 1H), 5.22 (m, 1H), 4.24 (m, 1H), 3.72 (s, 3H), 2.85 (m, 2H), 1.72-2.10 (m, 4H), 1.15 (m, 2H), 1.02 (m, 2H).

### EXAMPLE 169

### 1-cyclopropyl-8-methoxy-4-oxo-7-(4-((phenylacetyl)amino)-4,5,6,7-tetrahydro-1-benzothien-2-yl)-1,4-dihydro-3-guinolinecarboxylic acid

The desired product was prepared by substituting phenylacetic acid for acetic acid in Example 113. MS (DCI/NH₃) m/z 529 (M+H)⁺; ¹H NMR (300 MHz, DMSO-d₆) δ 14.92 (br s, 1H), 8.79 (s, 1H), 8.49 (d, 1H), 8.10 (d, 1H), 7.76 (d, 1H), 7.3 (s, 1H), 7.32 (m, 3H), 7.25 (m, 1H), 4.92 (m, 1H), 4.24 (m, 1H), 4.01 (s, 3H), 3.64 (s, 2H), 3.47 (d, 2H), 2.80 (m, 2H), 1.95 (m, 2H), 1.82 (m, 1H), 1.68 (m, 1H), 1.15 (m, 2H), 1.03 (m, 2H).

### EXAMPLE 170

### 1-cyclopropyl-7-(4-(3-furoylamino)-4,5,6,7-tetrahydro-1-benzothien-2-yl)-8-methoxy-4-oxo-1,4-dihydro-3-quinolinecarboxylic acid

The desired product was prepared by substituting 3-furoic acid for acetic acid in Example 113. MS (DCI/NH₃) m/z 505 (M+H)⁺; ¹H NMR (300 MHz, DMSO-d₆) δ 8.79 (s, 1H), 8.48 (d, 1H), 8.24 (d, 1H), 8.09 (d, 1H), 7.98 (d, 1H), 7.71 (d, 1H), 7.58 (s, 1H), 6.91 (d, 1H), 5.18 (m, 1H), 4.25 (m, 1H), 3.68 (s, 3H), 2.83 (m, 2H), 2.03 (m, 2H), 1.87 (m, 1H), 1.78 (m, 1H), 1.15 (m, 1H), 1.02 (m, 2H).

### EXAMPLE 171

### 1-cyclopropyl-8-methoxy-4-oxo-7-(4-((2-pyridinylcarbonyl)amino)-4,5,6,7-tetrahydro-1-benzothien-2-yl)-1 4-dihydro-3-quinolinecarboxylic acid trifluoroacetic acid salt

The desired product was prepared by substituting pyridine-2-carboxylic acid for acetic acid in Example 113. MS (DCI/NH₃) m/z 516 (M+H)⁺; ¹H NMR (300 MHz, DMSO-d₆) δ 8.74 (d, 1H), 8.62 (d, 1H), 8.54 (s, 1H), 8.13 (m, 1H), 8.03 (m, 1H), 7.92 (d, 1H), 7.75 (d, 1H), 7.61 (m, 1H), 7.59 (s, 1H), 5.20 (m, 1H), 4.06 (m, 1H), 3.65 (s, 3H), 2.84 (m, 2H), 1.83-2.08 (m, 4H), 1.09 (m, 2H), 0.92 (m, 2H).

### EXAMPLE 172

### 1-cyclopropyl-8-methoxy-4-oxo-7-(4-((1H-pyrazol-4-ylcarbonyl)amino)-4,5,6,7-tetrahydro-1-benzothien-2-yl)-1,4-dihydro-3-quinolinecarboxnlic acid bis(trifluoroacetic acid salt)

The desired product was prepared by substituting pyrazole-4-carboxylic acid for acetic acid in Example 113. MS (DCI/NH₃) m/z 505 (M+H)⁺; ¹H NMR (300 MHz, DMSO-d₆) δ 8.79 (s, 1H), 8.33 (d, 1H), 8.10 (m, 1H), 8.09 (d, 1H), 7.95 (d, 1H), 7.58 (s, 1H), 5.18 (m, 1H), 4.23 (m, 1H), 3.64 (s, 3H), 2.82 (m, 2H), 1.66-2.10 (m, 4H), 1.15 (m, 2H), 1.02 (m, 2H).

### EXAMPLE 173

### 7-(4-((D-aspartyl)amino)-4,5,6,7-tetrahydro-1-benzothien-2-yl)-1-cyclopropyl-8-methoxy-4-oxo-1,4-dihydro-3-quinolinecarboxylic acid, trifluoroacetic acid salt

The desired product was obtained as a by-product from Example 180. MS (DCI/NH₃) m/z 526 (M+H)⁺; ¹H NMR (300 MHz, DMSO-d₆) δ 8.79 (s, 1H), 8.18 (d, 1H), 7.98 (m, 1H), 7.44 (d, 1H), 4.96 (m, 1H), 4.08 (m, 1H), 3.76 (s, 3H), 3.,60 (m, 2H), 2.82 (m, 2H),m 1.98 (m, 2H), 1.86 (m, 1H), 1.72 (m, 1H), 1.14 (m, 2H), 1.00 (m, 2H).

### EXAMPLE 174

### 1-cyclopropyl-8-methoxy-7-(4-((N-methyl-D-leucyl)amino)-4,5,6,7-tetrahydro-1-benzothien-2-yl)-4-oxo-1,4-dihydro-3-quinolinecarboxylic acid, trifluoroacetic acid salt

The desired products were prepared by substituting N-(9-fluorenylmethoxycarbonyl)-N-methyl-L-leucine for N-(9-fluorenylmethoxycarbonyl)-glycine in Example 157. 4R-isomer: MS (DCI/NH₃) m/z 538 (M+H)⁺; ¹H NMR (300 MHz, DMSO-d₆) δ 14.98 (br s, 1H), 9.08 (d, 1H), 8.94 m, 1H), 8.80 (s, 1H), 8.11 (d, 1H), 7.83 (d, 1H), 7.47 (s, 1H), 5.03 (m, 1H), 4.25 (m, 1H), 3.68 (s, 3H), 2.83 (m, 2H), 1.54-2.07 (m, 8H), 1.18 (m, 2H), 1.06 (m, 2H), 0.92 (m, 6H). 4S-isomer: MS (DCINH₃) m/z 538 (M+H)⁺; ¹H NMR (300 MHz, DMSO-d₆) δ 14.97 (br s, 1H), 9.09 (d, 1H), 8.80 (s, 1H), 8.12 (d, 1H), 7.93 (d, 1H), 7.62 (s, 1H), 5.05 (m, 1H), 4.25 (m, 1H), 3.71 (m, 1H), 3.69 (s, 3H), 3.03 (m, 2H), 2.59 (s, 3H), 1.92 (m, 3H), 1.72 (m, 3H), 1.60 (m, 2H), 1.17 (m, 2H), 1.02 (m, 2H), 0.91 (m, 6H).

### EXAMPLE 175

### 7-(4-((D-norleucyl)amino)-4,5,6,7-tetrahydro-1-benzothien-2-yl)-1-cyclopropyl-8-methoxy-4-oxo-1,4-dihydro-3-quinolinecarboxylic acid, trifluoroacetic acid salt

The desired products were prepared by substituting and N-(9-fluorenylmethoxycarbonyl)-L-norleucine for N-(9-fluorenylmethoxycarbonyl)-glycine in Example 157. 4R-isomer: MS (DCI/NH₃) m/z 524 (M+H)⁺; ¹H NMR (300 MHz, DMSO-d₆) δ 14.98 (br s, 1H), 8.79 (s, 1H), 8.10 (d, 1H), 7.85 (d, 1H), 7.69 (s, 1H), 5.01 (m, 1H), 4.25 (m, 1H), 3.72 (m, 1H), 3.69 (s, 3H), 2.85 (m, 2H), 1.98 (m, 2H), 1.89 (m, 1H), 1.76 (m, 3H), 1.32 (m, 4H), 1.17 (m, 2H), 1.05 (m, 2H), 0.82 (m, 3H). 4S-isomer: MS (DCI/NH₃) m/z 540 (M+H)⁺; ¹H NMR (300 MHz, DMSO-d₆) δ 14.98 (br s, 1H), 8.82 (d, 1H), 8.79 (s, 1H), 7.94 (d, 1H), 7.57 (s, 1H), 5.01 (m, 1H), 4.26 (m, 1H), 3.77 (m, 1H), 3.70 (s, 3H), 2.84 (m, 2H), 1/97 (m, 2H), 1.87 (m, 1H), 1.71 (m, 3H), 1.32 (m, 4H), 1.18 (m, 2H), 1.03 (m, 2H), 0.88 (m, 3H).

### EXAMPLE 180

### 7-(4-((3-O-methyl-D-aspartyl)amino)-4,5,6,7-tetrahydro-1-benzothien-2-yl)-1-cyclopropyl-8-methoxy-4-oxo-1,4-dihydro-3-quinolinecarboxylic acid, trifluoroacetic acid salt

The desired product was prepared by substituting N-(9-fluorenylmethoxycarbonyl)-L-aspartic acid p-methyl ester for N-(9-fluorenylmethoxycarbonyl)-glycine in Example 157. Example 173 was isolated as a by-product. MS (DCI/NH₃) m/z 540 (M+H)⁺; ¹H NMR (300 MHz, DMSO-d₆) δ 14.98 (br s, 1H), 8.78 (s, 1H), 8.12 (dd, 1H), 7.97 (dd, 1H), 7.55 (dd, 1H), 4.98 (m, 1H), 4.26 (m, 1H), 4.12 (m, 1H), 3.68 (m, 6H), 2.95 (m, 1H), 2.82 (m, 2H), 1.98 (m, 2H), 1.88 (m, 1H), 1.70 (m, 1H), 1.12 (m, 2H), 0.98 (m, 2H).

### EXAMPLE 182

### 1-cyclopropyl-8-methoxy-4-oxo-7-((4E)-4-((3-pyridinylmethoxy)imino)-4,5,6,7-tetrahydro-1-benzothien-2-yl)-1,4-dihydro-3-quinolinecarboxylic acid

The desired product was obtained by substituting 3-picolylchloride hydrochloride, a reaction temperature of 70° C. and a reaction time of 7 hours for 4-picolylchloride hydrochloride, a reaction temperature of 50° C. and a reaction time of 12 hours, respectively in Example 131B. mp 181-183° C.; MS (APCI) m/z 516 (M+H)⁺; ¹H NMR (300 MHz, DMSO-d₆) δ 14.90 (s, 1H), 8.80 (s, 1H), 8.65 (m, 1H), 8.52 (m, 1H), 8.15 (m, 1H), 8.05 (d, 1H), 7.88 (s, 1H), 7.86 (m, 1H), 7.42 (dd, 1H), 5.23 (s, 2H), 4.25 (m, 1H), 3.70 (s, 3H), 2.40 (m, 2H), 2.30 (m, 2H), 1.95 (m, 2H), 1.15 (m, 2H), 1.07 (m, 2H).

### EXAMPLE 183

### 1-cyclopropyl-8-methoxy-4-oxo-7-((4E)-4-((2-pyridinylmethoxy)imino)-4,5,6,7-tetrahydro-1-benzothien-2-yl)-1,4-dihydro-3-quinolinecarboxylic acid

The desired product was obtained by substituting 2-picolylchloride hydrochloride and a reaction time of 8 hours for 4-picolylchloride hydrochloride and a reaction time of 12 hours, respectively in Example 131B. mp 178-179° C.; MS (APCI) m/z 516 (M+H)⁺; ¹H NMR (300 MHz, DMSO-d₆) δ 8.80 (s, 1H), 8.55 (d, 1H), 8.10 (d, 1H), 8.05 (d, 1H), 7.83 (s, 1H), 7.80 (dd, 1H), 7.40 (d, 1H), 7.32 (dd, 1H), 5.28 (s, 2H), 4.25 (m, 1H), 3.68 (s, 3H), 3.30 (s, 1H), 2.92 (dd, 2H), 2.80 (dd, 2H), 1.95 (m, 2H), 1.18 (m, 2H), 1.07 (m, 2H).

### EXAMPLE 185

### 1-cyclopropyl-7-(4-hydroxy-5-methyl-4,5,6,7-tetrahydro-1-benzothien-2-yl)-8-methoxy-4-oxo-1,4-dihydro-3-quinolinecarboxylic acid

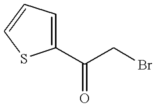

### EXAMPLE 185A

A mechanically stirred solution of 2-acetylthiophene (20.0 g, 0.160 mol) in 9:1 CH₂Cl₂:CH₃OH (400 ml) was treated with 4.0 mL conc. HCl followed by portionwise addition of 4-dimethylaminopyridinium tribromide (20.0 g, 0.170 mol). The resulting red orange solution was stirred 4 hours, concentrated, the residue slurried in diethyl ether and filtered. The filtrate was washed with water, brine, and dried (Na₂SO₄). Concetration gave the desired product (20 g, 63%) as an oil.

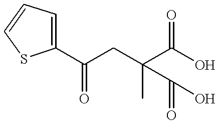

### EXAMPLE 185B

A mechanically stirred suspension of NaH (6.88 g of a 60% mineral oil suspension, 0.170 mol) in THF (70 ml) was cooled to 0° C., treated dropwise with diethyl methylmalonate (30.0 g, 0.170 mole), stirred for 20 minutes and treated dropwise with a solution of Example 185A (33.7 gm, 0.160 mol) in THF (110 ml). The resulting mixtue was allowed to warm to room temperature, stir 2 hours, was treated with water (20 ml), concentrated and partitioned between water and ethyl ether. The organic phase was washed with water, brine, and dried (Na₂SO₄).

A solution of the crude product above in 10% KOH (400 ml) and EtOH (200 ml) was heated to reflux for 1 hour and allowed to stir overnight at room temperature. The reaction mixture was poured into ice cold 6N HCl, the pH adjusted to 4.0 with conc. HCl, and extracted several times with ether. The combined organic layers were washed with water, brine, and dried (Na₂SO₄). After concentration, the desired product was obtained by crystallization from petroleum ether and ethyl ether (15.0 g, 40%) as a light brown solid.

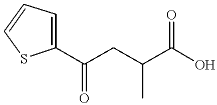

### EXAMPLE 185C

A solution of Example 185B (28.0 g, 0.115 mol) in diethylene glycol (100 mL) was heated at 175-180° C. for 20 minutes, allowed to cool to room temperature and was poured into ice/water containing a 5 mL of acetic acid. A brown tan precipitate was collected by filtration and recrystallized from hot water and ethanol to give the desired product (17.0 g, 75%) as white crystals.

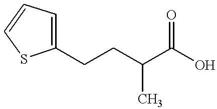

### EXAMPLE 185D

A solution of potassium hydroxide (16.4 g, 0.300 mol) in diethylene glycol (90 mL) was treated portionwise with Example 185C (16.1 g, 0.081 mol) followed by hydrazine monohydrate (8.20 g, 0.160 mol) and the resulting mixture heated to 200° C. for 2.5 hours. The reaction mixture was cooled to room temperature and partitioned between water and ethyl ether. The organic phase was washed with water, brine, dried (Na₂SO₄) and concentrated. The oily residue was distilled under reduced pressure (135-145° C., 0.2 torr) to give the desired product (13.8 g, 92 %).

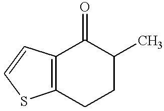

### EXAMPLE 185E

Neat polyphosphoric acid (100 g) was mechanically stirred, warmed to 70° C., and treated dropwise with a solution of Example 185D (9.70 g, 0.052 mol) in ethyl ether (10 ml). The reaction mixture was heated to 120° C. for 3 hours and poured into a mixture of ice and conc. HCl (40 mL). The resulting mixture was extracted with ethyl ether the organic phase washed with water, brine, and dried over (Na₂SO₄) and the crude residue purified by silica gel chromatography eluting with hexane/ethyl acetate (4:1) to give the desired product (3.0 g, 34%) as a clear oil.

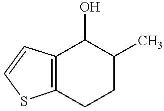

### EXAMPLE 185F

A solution of Example 185E (4.10 g, 0.025 mol) in EtOH (40 mL) was cooled to 0° C. and treated with NaBH₄ (2.50 g, 0.067 mol) and stirred for 2.5 hour at room temperature. The reaction was partitioned between 2% acetic acid and methylene chloride. The organic phase was washed with sat. NaHCO₃, brine, dried (Na₂SO₄) and concentrated to give the desired product as a yellow oil (3.75 g, 90%) which was used without further purification.

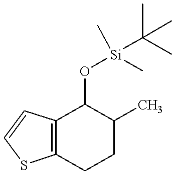

### EXAMPLE 185G

A solution of Example 185F (3.75 g, 0.022 mol), imidazole (3.0 g, 0.044 mol), and 0.020 g of N,N-dimethylaminopyridine in DMF (40 ml)was cooled to 0° C. and treated with tert-butyldimethylchlorosilane (6.7 g, 0.45 mol), warmed to room temperature, stirred for 20 hours, poured into 10% NH₄Cl, and extracted 2× with ethyl ether. The organic phase was washed with water, brine, dried (Na₂SO₄), and concentrated. The resulting residue was purified by silica gel chromatography eluting with hexane:ethyl acetate (7:1) to give the desired compound (6.1 g, 96%) as a yellow oil.

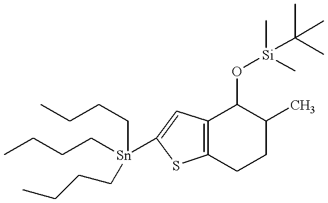

### EXAMPLE 185H

The desired product was prepared by substituting Example 185G for Example 218B in Example 218C and was used without further purification.

### EXAMPLE 1851

### ethyl 7-(4-((tert-butyl(dimethyl)silyl)oxy)-5-methyl-4,5,6,7-tetrahydro-1-benzothien-2-yl)-1-cyclopropyl-8-methoxy-4-oxo-1,4-dihydro-3-quinolinecarboxylate

The desired product was prepared by substituting Example 185H and ethyl-7-bromo-1-cyclopropyl-8-methoxy-4-oxo-1,4-dihydro-3-quinolinecarboxylate for ethyl-7-bromo-1-cyclopropyl-4-oxo-1,4-dihydro-3-quinolinecarboxylate and Example 1D, respectively in Example 1E and was purified by silica gel chromatography eluting with methylene chloride and then with a 3:97 methanol:methylene chloride gradient to give a yellow foam.

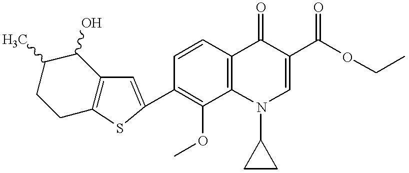

### EXAMPLE 185J

The desired product was prepared by substituting Example 1851 for Example 35D in Example 35E and was used without further purification.

### EXAMPLE 185K

### 1-cyclopropyl-7-(4-hydroxy-5-methyl4,5,6,7-tetrahydro-1-benzothien-2-yl)-8-methoxy-4-oxo-1,4-dihydro-3-quinolinecarboxylic acid

The desired product was prepared by substituting Example 185J for Example 2A in Example 2B and was purified by chromatography on silica gel eluting with hexane:acetone:methanol (50:45:5) to give light yellow solid. MS (ESI) m/z 426 (M+H)⁺; ¹H NMR (300 MHz, DMSO-d₆) δ 14.9 (br s, 1H), 8.79 (s, 1H), 8.12 (d, 1H,), 7.97 (t, 1H, maj, min), 7.73 (s, 1H, maj ), 7.69 (s, 1H, min), 5.22 (d, 1H, maj), 4.87 (d, 1H, min), 4.46 (m, 1H, min), 4.25 (m, 2H, maj, min), 4.14 (t, 1H, maj ), 3.60 (s, 3H), 2.82-2.78 (m, 2H, maj, min), 1.96-1.92 (m, 1H , min) 1.70-1.58 (m, 2H, maj, min), 1.14 (d, 3H, min), 1.06 (d, 3H, maj), 1.02 ( s, 2H, maj, min), 0.87 (t, 2H, maj, min).

### EXAMPLE 186

### 1-cyclopropyl-8-methoxy-7-(4-(((methylanilino)carbonyl)amino)-4,5,6,7-tetrahydro-1-benzothien-2-yl)-4-oxo-1,4-dihydro-3-guinolinecarboxlic acid

The Argonaut Quest 210 synthesizer was equipped with 5 ml reaction tubes. One tube was charged with PS-DIEA resin (Argonaut, 22-165 mesh, 1% DVB, 3.75 mmol/g loading) (0.072 g, 0.27 mmol) and a solution of Example 83A (0.040 g, 0.09 mmol) and 4-(dimethylamino)pyridine (0.010 g, 0.08 mmol) in 3 ml of 1:1/dichloromethane:dimethylacetamide was added. N-Methyl-N-phenylcarbamoyl chloride (0.018 mg, 0.108 mmol) was added and the mixture was shaken overnight at ambient temperature. PS-Trisamine resin (Argonaut, 100-200 mesh, 1% DVB, 4.06 mmol/g loading) (0.135 g, 0.55 mmol) was added and the mixture was shaken for 1 h. The mixture was filtered and washed with dichloromethane (2 x 2 ml). The resulting solution was concentrated to dryness and the residue was redissolved in 3 ml of 1:1/tetrahydrofuran:methanol. 2N aqueous sodium hydroxide solution (1 ml, 2.0 mmol) was added and the mixture was shaken overnight. The mixture was then neutralized with 4M hydrochloric acid in dioxane and concentrated to dryness. The residue was suspended in 1:1/dichloromethane:methanol (2 ml) and was filtered to remove any inorganics. The resulting clear solution was concentrated to dryness and was purified by preparative reverse phase HPLC (Waters Prep Nova-Pak HR C18 column, 25×100 mm, 6 μm, 60 Å, 0.01% trifluoroacetic acid in water:acetonitrile) to give the desired product (0.014 g, 28%) as an off-white solid. MS (DCI/NH₃) m/z 544 (M+H)⁺; ¹H NMR (300 MHz, DMSO-d₆) δ 8.79 (s, 1H), 8.14 (d, 1H), 7.98 (d, 1H), 7.63 (s, 1H), 7.38 (s, 1H), 6.15 (d, 1H), 4.82 (m, 1H), 4.26 (m, 1H), 3.78 (s, 3H), 3.26 (s. 3H), 2.76 (m, 2H), 1.95 (m, 2H), 1.75 (m, 2H), 1.18 (m, 2H), 1.05 (m, 2H).

### EXAMPLE 187

### 1-cyclopropyl-7-(4-(((diethylamino)carbonyl)amino)-4,5,6,7-tetrahydro-1-benzothien-2-yl -8-methoxy-4-oxo-1,4-dihydro-3-quinolinecarboxylic acid

The desired product was prepared by substituting N,N-diethylcarbamoyl chloride for N-methyl-N-phenylcarbamoyl chloride in Example 186. MS (DCI/NH₃) ml/z 510 (M+H)⁺; ¹H NMR (300 MHz, DMSO-d₆) δ 8.78 (s, 1H), 8.10 (d, 1H), 7.90 (d, 1H), 7.56 (s, 1H), 6.40 (d, 1H), 4.86 (m, 1H), 4.24 (m, 1H), 3.68 (s, 3H), 3.28 (m, 4H), 2.80 (m, 2H), 1.63-2.06 (m, 4H), 1.15 (m, 2H), 1.05 (m, 8H).

### EXAMPLE 188

### 1-cyclopropyl-7-(4-(((diisopropylamino)carbonyl)amino)-4,5,6,7-tetrahydro-1-benzothien-2-yl)-8-methoxy-4-oxo-1,4-dihydro-3-quinolinecarboxylic acid

The desired product was prepared by substituting N,N-diisopropylcarbamoyl chloride for N-methyl-N-phenylcarbamoyl chloride in Example 186. MS (DCI/NH₃) m/z 538 (M+H)⁺; ¹H NMR (300 MHz, DMSO-d₆) δ 8.78 (s, 1H), 8.10 (d, 1H), 7.84 (d, 1H), 7.58 (s, 1H), 6.18 (d, 1H), 4.86 (m, 1H), 4.24 (m, 2H), 3.75 (m, 2H), 3.68 (s, 3H), 2.80 (m, 2H), 2.05 (m, 1H), 1.93 (m, 1H), 1.78 (m, 2H), 1.22 (t, 6H), 1.14 (m, 2H), 1.04 (m, 2H).

### EXAMPLE 189

### 1-cyclopropyl-8-methoxy-7-(4-((4-morpholinylcarbonyl)amino)-4,5,6,7-tetrahydro-1-benzothien-2-yl)-4-oxo-1,4-dihydro-3-quinolinecarboxylic acid

The desired product was prepared by substituting morpholine-4-carbanoyl chloride for N-methyl-N-phenylcarbamoyl chloride in Example 186. MS (DCI/NH₃) m/z 524 (M+H)⁺; ¹H NMR (300 MHz, DMSO-d₆) δ 8.78 (s, 1H), 8.11 (d, 1H), 7.96 (d, 1H), 7.56 (s, 1H), 6.79 (d, 1H), 4.87 (m, 1H), 4.69 (m, 1H), 4.75 (m, 2H), 4.05 (m, 2H), 3.70 (m, 1H), 3.68 (s, 3H), 3.56 (m, 1H), 2.78 (m, 2H), 1.65-2.03 (m, 4H), 1.16 (m, 2H), 1.03 (m, 2H).

### EXAMPLE 190

### 1-cyclopropyl-8-methoxy-7-(4-((methoxycarbonyl)amino)-4,5,6,7-tetrahydro-1-benzothien-2-yl)-4-oxo-1,4-dihydro-3-quinolinecarboxylic acid

The desired product was prepared by substituting methyl chloroformate for N-methyl- N-phenylcarbamoyl chloride in Example 186. MS (DCI/NH₃) m/z 469 (M+H)⁺; ¹H NMR (300 MHz, DMSO-d₆) δ 8.79 (s, 1H), 8.11 (d, 1H), 7.99 (d, 1H), 7.59 (s, 1H), 4.68 (m, 1H), 4.25 (m, 1H), 3.68 (s, 3H), 3.59 (s, 3H), 2.78 (m, 2H), 1.64-2.02 (m, 4H), 1.17 (m, 2H), 1.03 (m, 2H).

### EXAMPLE 191

### 7-(4-(((benzyloxy)carbonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting benzyl choroformate for N-methyl-N-phenylcarbamoyl chloride in Example 186. MS (DCI/NH₃) m/z 545 (M+H)⁺; ¹H NMR (300 MHz, DMSO-d₆) δ 8.78 (s, 1H), 8.10 (d, 1H), 7.90 (d, 1H), 7.72 (d, 1H), 7.55 (s, 1H), 7.49 (m, 3H), 7.45 (m, 1H), 5.11 (dd, 2H), 4.71 (m, 1H), 4.25 (m, 2H), 3.67 (s, 3H), 2/79 (m, 2H), 1.64-2.02 (m, 4H), 1.15 (m, 2H), 1.02 (m, 2H).

### EXAMPLE 192

### 1-cyclopropyl-7-(4-((isobutoxycarbonyl)amino)-4,5,6,7-tetraydro-1-benzothien-2-yl)-8-methoxy-4-oxo-1,4-dihydro-3-quinolinecarboxylic acid

The desired product was prepared by substituting isobutyl chloroformate for N-methyl-N-phenylcarbamoyl chloride in Example 186. MS (DCI/NH₃) m/z 511 (M+H)⁺; ¹H NMR (300 MHz, DMSO-d6) δ 8.78 (s, 1H), 8.10 (d, 1H), 7.94 (d, 1H), 7.58 (s, 1H), 7.54 (d, 1H), 4.68 (m, 1H), 4.26 (m, 1H), 3.82 (m, 2H), 3.68 (s, 3H), 2.78 (m, 2H), 1.63-2.04 (m, 5H), 1.16 (m, 2H), 1.04 (m, 2H), 0.91 (d, 6H).

### EXAMPLE 193

### 1-cyclopropyl-7-(4-((ethoxycarbony)amino)-4,5,6,7-tetrahydro-1-benzothien-2-yl)-8-methoxy-4-oxo-1,4-dihydro-3-quinolinecarboxylic acid

The desired product was prepared by substituting ethyl chloroformate for N-methyl-N-phenylcarbamoyl chloride in Example 186. MS (DCI/NH₃) m/z 483 (M+H)⁺; ¹H NMR (300 MHz, DMSO-d₆) δ 8.78 (s, 1H), 8.10 (d, 1H), 7.96 (d, 1H), 7.59 (s, 1H), 7.52 (d, 1H), 4.68 (m, 1H), 4.26 (m, 1H), 4.05 (q. 2H), 3.68 (s, 3H), 2.78 (m, 2H), 1.98 (m, 2H), 1.80 (m, 1H), 1.70 (m, 1H), 1.21 (t, 3H), 1.15 (m, 2H), 1.02 (m, 2H).

### EXAMPLE 194

### 7-(4-((butoxycarbonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting n-butyl chloroformate for N-methyl-N-phenylcarbamoyl chloride in Example 186. MS (DCI/NH₃) m/z 511 (M+H)⁺; ¹H NMR (300 MHz, DMSO-d₆) δ 14.92 (br s, 1H), 8.78 (s, 1H), 8.10 (d, 1H), 7.96 (d, 1H), 7.58 (s, 1H), 7.52 (d, 1H), 4.68 (m, 1H), 4.23 (m, 1H), 4.02 (m, 2H), 3.68 (s, 3H), 2.78 (m, 2H), 1.97 (m, 2H), 1.80 (m, 1H), 1.70 (m, 1H), 1.56 (m, 2H), 1.46 (m, 2H), 1.15 (m, 2H), 1.03 (m, 2H), 0.90 (t, 3H).

### EXAMPLE 195

### 7-(4-(((4-chlorobutoxy)carbonyl)amino)-4,5,6,7-tetrahydro-1-benzothien-2-yl)-1-cyclopropyl-8-methoxy-4-oxo-1,4-dihydro-3-quinolinecarboxylic acid

The desired product was prepared by substituting 4-chlorobutyl chloroformate for N-methyl-N-phenylcarbamoyl chloride in Example 186. MS (DCI/NH₃) m/z 546 (M+H)⁺; ¹H NMR (300 MHz, DMSO-d₆) δ 8.79 (s, 1H), 8.12 (d, 1H), 7.96 (d, 1H), 7.59 (s, 1H), 6.79 (d, 1H), 4.87 (m, 1H), 4.25 (m, 1H), 3.68 (s, 3H), 3.55 (m, 4H), 2.80 (m, 2H), 1.64-2.06 (m, 4H), 1.18 (m, 2H), 1.03 (m, 2H).

### EXAMPLE 196

### 1-cyclopropyl-7-((5E/Z)-5-(hydroxymethylene)-4-oxo-4,5,6,7-tetrahydro-1-benzothien-2-yl)-8-methoxy-4-oxo-1,4-dihydro-3-quinolinecarboxylic acid

A solution of sodium methoxide (0.30 g, 5.6 mmol) and ethyl formate (0.45 mL, 5.6 mmol) in toluene (10 mL) at 0° C. was treated with Example 50A (0.82 g, 1.9 mmol). The reaction mixture was stirred at 0° C. for 3 hours then at room temperature for 2 hours and partitioned between 1M sodium hydroxide and dichloromethane. The aqueous layer was acidified with IM hydrochloric acid and the product extracted into dichloromethane, dried (Na₂SO₄), filtered, and concentrated to yield (0.10 g, 12%) of the desired compound. MS (DCI/NH₃) m/z 438 (M+H)⁺; ¹H NRM (300 MHz, CDCl₃) δ 8.93 (s, 1H), 8.28 (d, 1H), 7.98 (s, 1H), 7.82 (d, 1H), 7.51 (m, 1H), 4.14 (m, 1H), 3.70 (s, 3H), 3.10 (t, 2H), 2.77 (m, 2H), 1.31 (m, 2H), 1.08 (m, 2H).

### EXAMPLE 198

### 1-cyclopropyl-7-(4-(3-hydroxy-1-azetidinyl)-4,5,6,7-tetrahydro-1-benzothien-2-yl)-8-methoxy-4-oxo-1,4-dihydro-3-quinolinecarboxylic acid hydrochloride

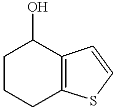

### EXAMPLE 198A

A solution of 4-keto-4,5,6,7-tetrahydrothianaphthene (5.0 g, 32.85 mmol) in methanol (100 mL) at 0° C. was treated with sodium borohydride (2.3 g, 60.80 mmol) and stirred 2 hours. The reaction mixture was partitioned between dichloromethane and water, the aqueous layer extracted with dichloromethane, the combined organic layers dried (Na₂SO₄) and concentrated to yield the desired compound (5.06 g, 100%).

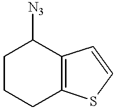

### EXAMPLE 198B

The desired product was prepared by substituting Example 198A and dichloromethane as the reaction solvent for Example 37A and 1:1 toluene:dichloromethane as reaction solvent, respectively in Example 37B and was used without further purification.

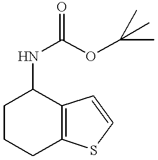

### EXAMPLE 198C

The desired product was prepared by substituting Example 198B for Example 201C Example 201D.

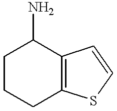

### EXAMPLE 198D

A solution of Example 198C (2.64 g, 10.4 mmol) in dichloromethane (20 mL) was treated with 4M HCl in dioxane (5.2 mL) at room temperature for 4 hours. The resulting precipitate was filtered, rinsed with diethyl ether, suspended in a 1:1 mixture of dichloromethane and methanol (50 mL) and treated with Amberlite IRA-400 (OH) ion exchange resin at room temperature for 2 hours. The resin was filtered and the filtrate concentrated to yield the desired product (0.72 g, 45%).

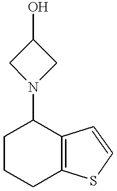

### EXAMPLE 198E

A solution of Example 198D (0.72 g, 4.7 mmol) in isopropanol (20 mL) was treated with epichlorohydrin (0.48 mL, 6.1 mmol) and heated to 75° C. for 26 hours. The reaction mixture was partitioned between 5% sodium bicarbonate solution and dichloromethane, the organic phase dried (Na₂SO₄) and concentrated to yield the desired compound (0.47 g, 48%).

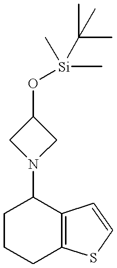

### EXAMPLE 198F

A solution of Example 198E (0.47 g, 2.2mmol) in dichloromethane (30 mL) was cooled to 0° C., treated with tert-butyldimethylsilyl chloride (2.5 mL of a 1.0M solution in dichloromethane, 2.5 mmol) and imidazole (0.28 g, 4.0 mmol), and stirred for 3 hours at this temperature followed by 20 hours at room temperature. The reaction mixture was partitioned between water and dichloromethane, dried (Na2SO4) and the concentrated residue purified by silica gel chromatography eluting with 2% methanol in dichloromethane to yield the desired compound (0.58 g, 80%).

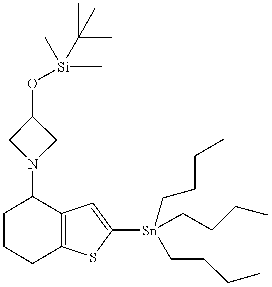

### EXAMPLE 198G

The desired product was prepared by substituting Example 198F for Example 218B in Example 218C.

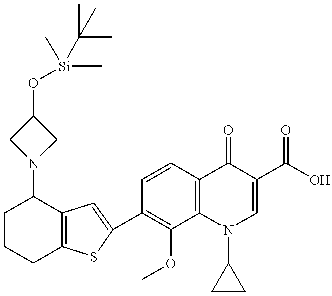

### EXAMPLE 198H

The desired product was prepared by substituting Example 198G, ethyl 7-bromo-1-cyclopropyl-8-methoxy-4-oxo-1,4-dihydro-3-quinolinecarboxylate and a reaction time of 3 hours for Example 1D, ethyl 7-bromo-1-cyclopropyl-4-oxo-1,4-dihydro-3-quinolinecarboxylate and a reaction time of 24 hours, respectively in Example 1E.

### EXAMPLE 198I

### 1-cyclopropyl-7-(4-(3-hydroxy-1-azetidinyl)-4,5,6,7-tetrahydro-1-benzothien-2-yl)-8-methoxy-4-oxo-1,4-dihydro-3-guinolinecarboxvlic acid hydrochloride

The desired product was prepared by substituting Example 198H for Example 35D in Example 35E and the crude product treated with 4M HCl in dioxane. MS (DCI/NH₃) m/z 467 (M+H)⁺; ¹H NMR (300 MHz, CD_{3l 0 D) δ} 8.96 (s, 1H), 8.23 (d, 1HO, 7.95 (d, 1H), 7.74 (s, 1H), 4.60 (m, 1H), 4.40 (m, 2H), 4.28 (m, 2H), 4.13 (m, 1H), 3.94 (m, 2H), 3.72 (s, 3H), 2.97 (m, 2H), 2.03 (m, 4H), 1.28 (m, 2H), 1.06 (m, 2H).

### EXAMPLE 199

### 7-(4-amino-5-methyl-4,5,6,7-tetrahydro-1-benzothien-2-yl)-1-cyclopropyl-8-methoxy-4-oxo-1,4-dihydro-3-quinolinecarboxylic acid hydrochloride

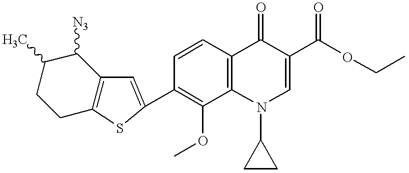

### EXAMPLE 199A

The desired product was prepared by substituting Example 185J for Example 37A in Example 37B and was purified by chromatography on silica gel eluting with CH₂Cl₂:CH₃OH (98:2) to give a yellow solid.

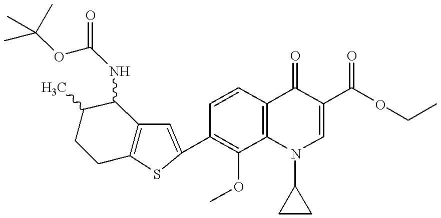

### EXAMPLE 199B

The desired product was prepared by substituting Example 199A for Example 201C in Example 201D and was purified by chromatography on silica gel eluting with CH₂Cl₂:CH₃OH (96:4) to give a yellow oil.

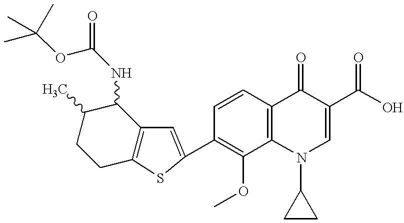

### EXAMPLE 199C

The desired product was prepared by substituting Example 199B for Example 2A in Example 2B.

### EXAMPLE 199D

### 7-(4-amino-5-methyl-4,5,6,7-tetrahydro-1-benzothien-2-yl)-1-cyclopro]yl-8-methoxy-4-oxo-1,4-dihydro-3-quinolinecarboxylic acid hydrochloride

The desired product was prepared by substituting Example 199C for Example 40B in Example 40C. MS (ESI) m/z 425 (M+H+)⁺; ¹H NMR (300 MHz, DMSO-d₆) δ 8.80 (s, 1H), 8.5 (br s, 2H, maj, min), 8.49 (br s, 1H, maj, min), 8.17 (d, 1H, maj, min), 8.11 (s, 1H, maj), 7.96 (s, 1H, min), 7.91 (d, 1H, maj, min), 4.40 (br s, 1H, min), 4.26 (m, 2H, maj, min), 4.09 (t, 1H, maj), 3.74 (s, 3H, min), 3.70 (s, 3H, maj), 2.92-2.84 (m, 2H, maj, min), 2.10-2.08 (m, 2H, maj, min), 1.86-1.84 (m, 1H, min), 1.72-1.68 (m, 1H, maj), 1.14 (d, 3H, maj, min), 1.08 (d, 2H, maj, min), 1.02 (br s, 2H).

### EXAMPLE 200

### 7-(4-amino-4,5,6,7-tetrahydro-1-benzothien-2-yl)-1-cyclopropyl-4-oxo-1,4-dihydro[1,8]naphthyridine-3-carboxylic acid hydrochloride

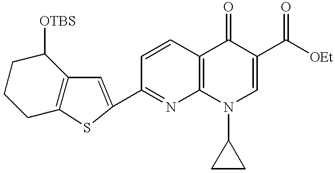

### EXAMPLE 200A

The desired product was prepared by substituting Example 38B and ethyl 1-cylopropyl-7-chloro-4-oxo-1,4-dihydro[1,8]naphthyridine-3-carboxylate for Example 1D and ethyl 1-cyclopropyl-7-bromo-8-methoxy-4-oxo-1,4-dihydroquinoline-3-carboxylate, respectively in Example 1E and the crude material purified by silica gel chromatography eluting with 2:1 hexane:acetone.

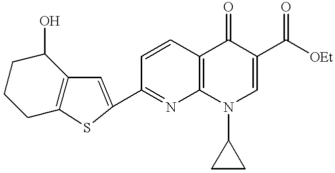

### EXAMPLE 200B

The desired product was prepared by substituting Example 200A for Example 35D in Example 35E to give a yellow solid that was used without further purification.

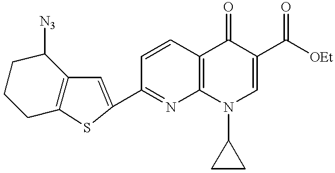

### EXAMPLE 200C

The desired product was prepared by substituting Example 200B and 1:10 THF/dichloromethane as reaction solvent for Example 37A for 1:1 toluene/dichloromethane as reaction solvent, respectively in Example 37B.

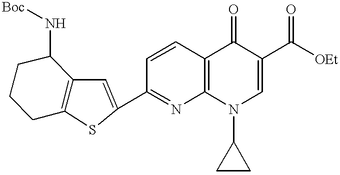

### EXAMPLE 202D

The desired product was prepared by substituting Example 200C for Example 201C in Example 201D and the crude product triturated in 3:1 hexane/acetone and filtered to provide a cream colored solid.

### EXAMPLE 200E

### 7-(4-amino-4,5,6,7-tetrahydro-1-benzothien-2-yl)-1-cyclopropyl-4-oxo-1,4-dihydro1,8]naphthyridine-3-carboxylic acid hydrochloride

The desired product was prepared by substituting Example 200D for Example 48C and THF for EtOH in Example 48D and the reaction mixture filtered to provide a yellow solid. MS (APCI) m/z 382 (M+H)⁺; 1H NMR (300 MHz, DMSO-d₆) δ 14.75 (bs, 1H), 8.80 (s, 1H), 8.73 (d, 1H), 8.64 (s, 3H), 8.33 (s, 1H), 7.92 (d, 1H), 4.41 (m, 1H), 3.82 (m, 1H), 2.86 (m, 2H), 2.12 (m, 2H), 1.86 (m, 2H), 1.31 (m, 2H), 1.15 (m, 2H).

### EXAMPLE 201

### 7-(7-amino-4,5,6,7-tetrahydro-1-benzothien-2-yl)-1-cyclopropyl-8-methoxy-4-oxo-1,4-dihydro-3-quinolinecarboxylic acid hydrochloride

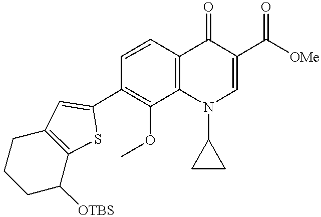

### EXAMPLE 201A

The desired product was prepared by substituting Example 41B and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E followed by substituting the resulting crude product for Example 38C in Example 39A.

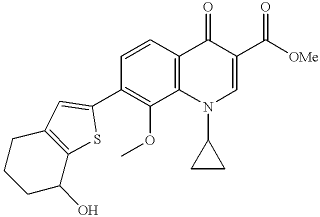

### EXAMPLE 201B

The desired product was prepared by substituting Example 201A for Example 35D in Example 35E and was purified by silica gel chromatography eluting with hexane then 50% acetone in hexane to provide the desired product.

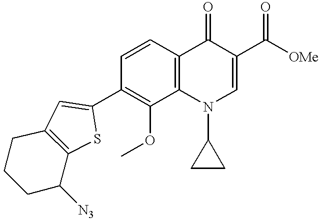

### EXAMPLE 201C

The desired product was prepared by substituting Example 201B for Example 37A in Example 37B and was purified by silica gel chromatography eluting with hexane then 50% acetone in hexane to provide the desired product.

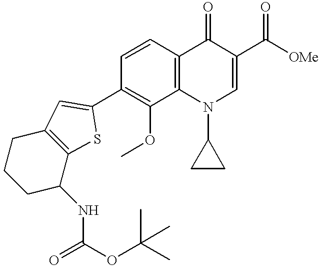

### EXAMPLE 201D

A solution of Example 201C (0.751 g, 1.70 mmol), palladium hydroxide (0.075 g), and di-t-butyl dicarbonate (1.85 g, 8.50 mmol) in ethanol (20 mL) was treated with triethyl silane (1.1 mL, 6.80 mmol) and stirred overnight. The reaction mixture was filtered. The filtrate was partitioned between CH₂Cl₂ and water. The aqueous phase was extracted with CH₂Cl₂, the combined organic phases washed with water, brine, dried (Na₂SO₄), and concentrated. The residue was purified by silica gel chromatography eluting with a gradient of hexane to 50% acetone in hexane to provide the desired product (0.540 g, 1.00 mmol) as a yellow solid.

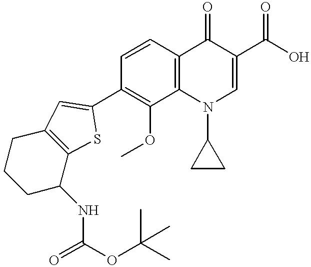

### EXAMPLE 201E

The desired product was prepared by substituting Example 201D for Example 2A in Example 2B.

### EXAMPLE 201F

### 7-(7-amino-4,5,6,7-tetrahydro-1-benzothien-2-yl)-1-cyclopronyl-8-methoxy-4-oxo-1,4-dihydro-3-quinolinecarboxylic acid hydrochloride

The desired product was prepared by substituting Example 201E for Example 40B in Example 40C. Mp 174-178° C.; MS (DCI/NH3) m/z 411 (M+1)⁺; ¹H NMR (300 MHz, DMSO-d₆) δ 14.86 (br s, 1H), 8.82 (s, 1H), 8.46 (br s, 2H), 8.15 (d, 1H), 8.04 (d, 1H), 7.67 (s, 1H), 4.62 (m, 1H), 4.27 (m, 1H), 3.71 (s, 3H), 2.72 (m, 2H), 2.14-1.82 (m, 4H), 1.16-1.04 (m, 4H); ¹³C NMR (75 MHz, DMSO-d₆) δ 177.2, 165.6, 151.7, 147.0, 139.6, 136.3, 136.0, 134.5, 132.8, 128.7, 126.5, 125.8, 121.6, 107.8, 66.4, 62.9, 45.1, 27.8, 24.8, 18.3, 9.2, 9.1; IR (mic) 3420 (COOH), 1730 (C═O) cm⁻¹; Anal. calcd for C₂₂H₂₃N₂O₄SCl.0.5 H₂O: C, 57.95; H, 5.30; N, 6.14; found: C, 57.95; H, 5.25; N, 6.25.

### EXAMPLE 202

### 1-cyclopropyl-7-(7-hydroxy-4,5,6,7-tetrahydrothieno[3,2-c]pyridin-2-yl)-8-methoxy-4-oxo-1,4-dihydro-3-quinolinecarboxylic acid

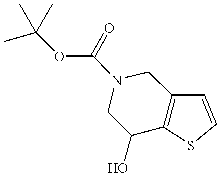

### EXAMPLE 202A

A solution of 7-hydroxy-4,5,6,7-tetrahydrothieno(3,2-c)pyridine (prepared by the method of Maffrand, et. al.; J. Heterocycl. Chem. 13; 1976; 1347-1349) (2.10 g, 13.50 mmol) in CH₂Cl₂ (20 mL) and di-tert-butyldicarbonate (3.54 g, 16.2 mmol) was stirred overnight at ambient temperature. The reaction mixture was poured over water, extracted with diethyl ether and the combined organic phases dried (Na₂SO₄) and the crude product purified by silica gel chromatography eluting with 15% Et₂O in hexanes to afford the desired product (1.03 g, 31% yield) as a clear oil.

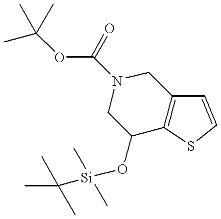

### EXAMPLE 202B

The desired product was prepared by substituting Example 202A for Example 185F in Example 185G.

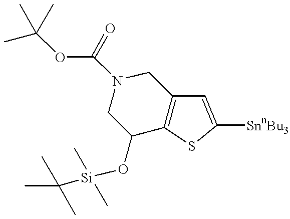

### EXAMPLE 202C

The desired product was prepared by substituting Example 202B for Example 11C in Example 11D.

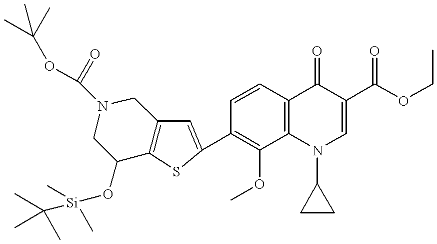

### EXAMPLE 202D

The desired product was prepared by substituting Example 202C and ethyl 7-bromo-1-cyclopropyl-8-methoxy-4-oxo-1,4-dihydro-3-quinolinecarboxylate for Example 1D and 7-bromo-1-cyclopropyl-4-oxo-1,4-dihydro-3-quinolinecarboxylate, respectively in Example 1E and was purified by silica gel chromatography eluting with a gradient from 20-50% acetone in hexanes.

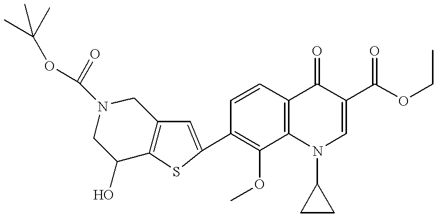

### EXAMPLE 202E

The desired product was prepared by substituting Example 202D for Example 35D in Example 35E and was purified by silica gel chromatography eluting with 25% acetone in hexanes followed by 4% methanol in CH₂Cl₂.

### EXAMPLE 202F

### 1-cyclopropyl-7-(7-hydroxy-4,5,6,7-tetrahydrothieno[3,2-c]pyridin-2-yl)-8-methoxy-4-oxo-1,4-dihydro-3-quinolinecarboxylic acid

The desired product was prepared by substituting Example 202E for Example 59C in Example 59D to give the product as a colorless solid. MS (DCI/NH₃) m/z 413 (M+H)⁺; ¹H-NMR (300 MHz, d₆-DMSO) δ 9.75 (br s, 1H), 9.40 (br s, 1H), 8.80 (s, 1H), 8.15 (d, 1H), 7.95 (d, 1H), 7.69 (s, 1H), 5.05 (t, 1H), 4.25 (m, 3H), 3.70 (s, 3H), 3.60-3.20 (br m, 2H), 1.17 (m, 2H), 1.05 (m, 2H).

### EXAMPLE 203

### 1-cyclopropyl-8-methoxy-4-oxo-7-(7-oxo-4,5,6,7-tetrahydrothieno[3,2-c]pyridin-2-yl)-1,4-dihydro-3-quinolinecarboxylic acid hydrochloride

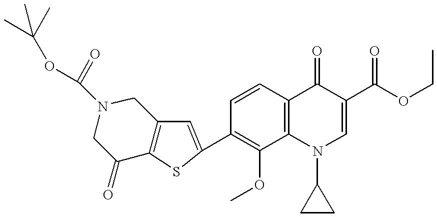

### EXAMPLE 203A

The desired product was prepared by substituting Example 202D for Example 41C in Example 32451 1A and was purified by silica gel chromatography eluting with a gradient from 20-30% acetone in hexanes step.

### EXAMPLE 203B

### 1-cyclopropyl-8-methoxy-4-oxo-7-(7-oxo-4,5,6,7-tetrahydrothieno[3,2-c]pyridin-2-yl)-1,4-dihydro-3-quinolinecarboxvlic acid hydrochloride

The desired product was prepared by substituting Example 203A for Example 59C in Example 59D. MS (DCI/NH₃) m/z 411 (M+H)⁺; ¹H-NMR (300 MHz, d₆-DMSO) δ 10.26 (br s, 1H), 8.83 (s, 1H), 8.23 (d, 1H), 8.11 (d, 1H), 8.03 (s, 1H), 4.58 (s, 2H), 4.28 (m, 1H), 4.12 (s, 2H), 3.75 (s, 3H), 1.18 (m, 2H), 1.08 (m, 2H).

### EXAMPLE 204

### 1-cyclopropyl-7-(5,5-difluoro-4-hydroxy-4,5,6,7-tetrahydro-1-benzothien-2-yl)-8-methoxy-4-oxo-1,4-dihydro-3-quinolinecarboxylic acid

The desired product was prepared by substituting Example 206E for Example 2A in Example 2B and the resulting crude product triturated in hot 33% acetone in hexanes and filtered to give a buff solid. MS (APCI) m/z 448 (M+H)⁺; ¹H NMR (300 MHz, DMSO-d₆) δ 8.80 (s, 1H), 8.13 (d, 1H), 8.02 (d, 1H), 7.73 (s, 1H), 6.13 (m, 1H), 4.71 (m, 1H), 4.27 (m, 1H), 3.69 (s, 3H), 3.04-2.90 (m, 2H), 2.50-2.21 (m, 2H), 1.15 (m, 2H), 1.04 (m, 2H).

### EXAMPLE 206

### 7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid

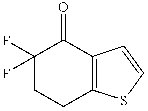

### EXAMPLE 206A

A solution of 4-keto-4,5,6,7-tetrahydrothianapthene (3.16 g, 20.8 mmol) in THF (150 ml) was treated with N-fluorobenzenesulfonamide (15.06 g, 47.8 mmol), cooled to −78° C., treated dropwise with lithium bis(trimethylsilyl)amide (52.0 ml of a IM THF solution, 51.9 mmol), stirred 1 hour at this temperature and allowed to come to room temperature and stir for 14 hours. The resulting reaction mixture was treated with water (2 ml) and partitioned between saturated aqueous ammonium chloride and ethyl acetate. The aqueous phase was extracted with ethyl acetate and the combined organic layers washed with saturated aqueous NaHCO3, water, brine and dried (MgSO4). After concentration, the crude residue was purified by column chromatography on silica gel eluting with 10% then 20% ethyl acetate in hexanes to give the desired product (2.96 g, 76%) as an amber oil.

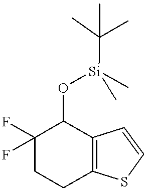

### EXAMPLE 206B

The desired product was prepared by substituting Example 206A for Example 21 8A in Example 218B and the crude residue purified by silica gel chromatography eluting with 10% ethyl acetate in hexanes.

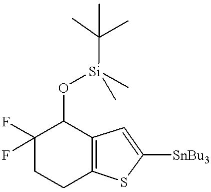

### EXAMPLE 206C

The desired product was prepared by substituting Example 206B for Example 218B in Example 218C and was used without fuirther purification.

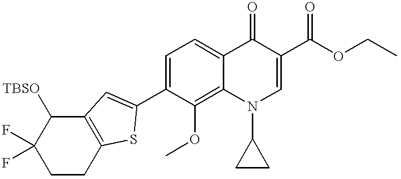

### EXAMPLE 206D

The desired product was prepared by substituting Example 206C and ethyl 7-bromo-1-cyclopropyl-8-methoxy-4-oxo-1,4-dihydro-3 -quinolinecarboxylate for Example 1D and 7-bromo-1-cyclopropyl-4-oxo-1,4-dihydro-3-quinolinecarboxylate, respectively in Example 1E and the crude residue purified by silica gel chromatography eluting with hexanes, 20% acetone in hexanes, 25% acetone in hexanes then 33% acetone in hexanes.

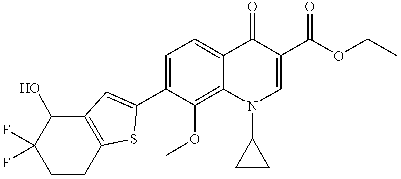

### EXAMPLE 206E

The desired product was prepared by substituting Example 206D for Example 35D in Example 35E and the crude alcohol carried on without purification.

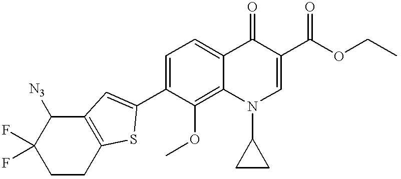

### EXAMPLE 206F

A solution of Example 204E (1.41 g, 2.97 mmol) in CH₂Cl₂ (50 ml) was treated with diphenylphosphorylazide (2.45 g, 8.91 mmol) followed by DBU (1.36 g, 8.91 mmol) and stirred at room temperature for 16 hours. The reaction mixture was partitioned between ethyl acetate and saturated aqueous NH₄Cl and the aqueous layer extracted with ethyl acetate. The combined organic layers were washed with 10% H₃PO₄, water, brine and dried (MgSO₄). Concentration gave the phosphonate as a thick amber oil that was carried on without purification.

A solution of the above phosphonate in DMSO (50 ml) was treated with sodium azide (1.93 g, 29.7 mmol) and heated to 85° C. for 14h. The reaction mixture was poured into 700 ml water, diluted with 200 ml ethyl acetate and NaCl added slowly to the stirred mixture until the layers resolved. The layers were separated and the aqueous phase extracted with ethyl acetate and the combined organic layers washed with water, brine and dried (MgSO₄). The concentrated residue was purified by silica gel chromatography eluting with a gradient from 25% to 33% acetone in hexanes to give the desired product (0.527 g, 36%) as a tan foam.

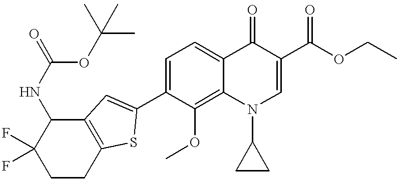

### EXAMPLE 206G

The desired product was prepared by substituting Example 206F for Example 201C in Example 201C and the crude product purified by silica gel chromatography eluting with 33% acetone in hexanes.

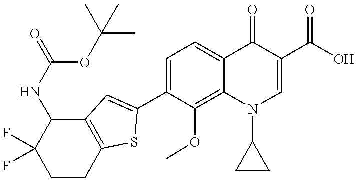

### EXAMPLE 206H

The desired product was prepared by substituting Example 206G for Example 2A in Example 2B and the crude product purified by silica gel chromatography eluting with 3% methanol in CH2Cl2.

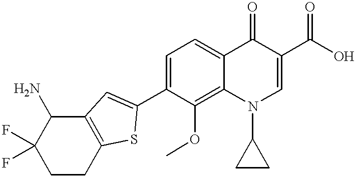

### EXAMPLE 206I

### 7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid

The desired product was prepared by substituting Example 206H for Example 40B in Example 40C. MS (ESI) m/z 447 (M+H)⁺; ¹H NMR (300 MHz, DMSO-d₆) δ 9.17 (br s, 3H), 8.81 (s, 1H), 8.22 (d, 1H), 8.09 (s, 1H), 7.87 (d, 1H), 5.03 (m, 1H), 4.27 (m, 1H), 3.71 (s, 3H), 3.12 (m, 2H), 2.75-2.50 (m, 2H), 1.16 (m, 2H), 1.05 (m, 2H).

### EXAMPLE 207

### 1-cyclopropyl-7-(5-(hydroxymethyl)-5-methyl-4-oxo-4,5,6,7-tetrahydro-1-benzothien-2-yl)-8-methoxy-4-oxo-1,4-dihydro-3-quinolinecarboxylic acid

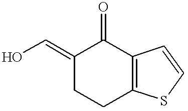

### EXAMPLE 207A

The desired product was prepared by substituting 4-Keto-4,5,6,7-tetrahydrothianaphthene for Example 50A in Example 196A.

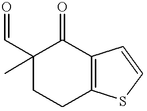

### EXAMPLE 207B

A solution of Example 207A (1.73 g, 9.6 mmol) in acetone (100 mL) was treated with potassium carbonate (3.97 g, 28.8 mmol) and methyl iodide (0.78 mL, 12.5 mmol) at room temperature for 28 hours. The reaction mixture was partitioned between water and dichloromethane, dried (Na₂SO₄), filtered, concentrated, and purified by silica gel chromatography eluting with 1% methanol in dichloromethane to yield 1.05 g (56%) of the desired compound.

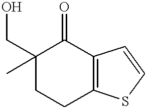

### EXAMPLE 207C

A solution of Example 207B (1.03 g, 5.3 inmol) in dichloromethane (40 mL) was treated with acetic acid (2 drops) and sodium cyanoborohydride (0.30 g, 6.1 mmol) at room temperature for 3 hours. The reaction mixture was partitioned between dichloromethane and water, dried (Na₂SO₄), concentrated and purified by silica gel chromatography eluting with a gradient of 1% to 4% methanol in dichloromethane to yield (0.26 g, 13%) of the desired compound.

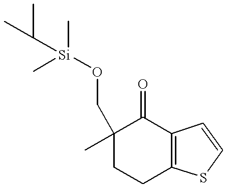

### EXAMPLE 207D

The desired product was prepared by substituting Example 207C for Example 198E in Example 198F.

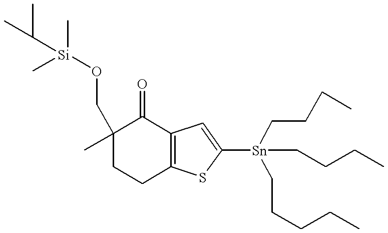

### EXAMPLE 207E

The desired product was prepared by substituting Example 207D for Example 218B in Example 218C.

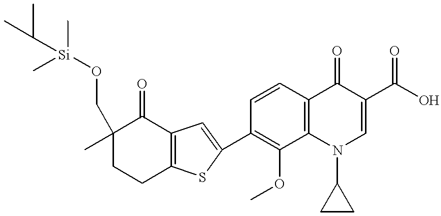

### EXAMPLE 207F

The desired product was prepared by substituting Example 207E, ethyl 7-bromo-1-cyclopropyl-8-methoxy-4-oxo-1,4-dihydro-3-quinolinecarboxylate and a reaction time of 4 hours for Example 1D, ethyl 7-bromo-1-cyclopropyl-4-oxo-1,4-dihydro-3-quinolinecarboxylate and a reaction time of 24 hours in Example 1E.

### EXAMPLE 207G

### 1-cyclopropyl-7-(5-(hydroxymethyl)-5-methyl-4-oxo-4,5,6,7-tetrahydro-1-benzothien-2-yl)-8-methoxy-4-oxo-1,4-dihydro-3-quinolinecarboxylic acid

The desired product was prepared by substituting Example 207F for Example 35D in Example 35E and the crude product triturated with 1:1 diethyl ether:ethanol. MS (ESI+) m/z 454 (+H)⁺; ¹H NMR (300 MHz, DMSO-d₆) δ 8.81 (s, 1H), 8.11 (d, 1H), 7.96 (d, 1H), 7.51 (s, 1H), 4.27 (m, 1H), 4.00 (s, 2H), 3.70 (s, 3H), 3.17 (m, 2H), 2.35 (m, 2H), 1.96 (m, 2H), 1.17 (m, 2H), 1.05 (m, 2H), 0.81 (s, 3H).

### EXAMPLE 208

### 7-(7-amino-4,5,6,7-tetrahydrothieno[3,2-c]pyridin-2-yl)-1-cyclopropyl-8-methoxy-4-oxo-1,4-dihydro-3-quinolinecarboxylic acid

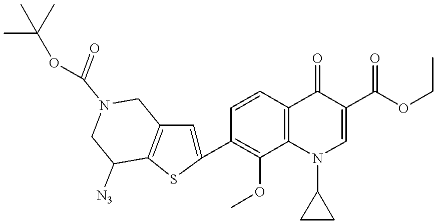

### EXAMPLE 208A

The desired product was prepared by substituting Example 202E for Example 37A in Example 37B and was purified by silica gel chromatography eluting with 20% acetone in hexanes.

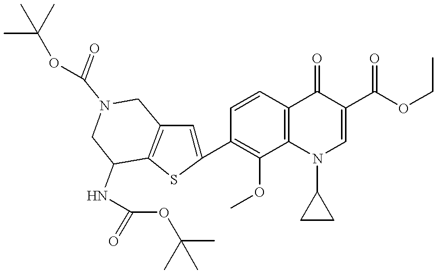

### EXAMPLE 208B

The desired product was prepared by substituting Example 208A for Example 353365C in Example 353365D and was purified by silica gel chromatography using 20% acetone in hexanes.

### EXAMPLE 208C

### 7-(7-amino-4,5,6,7-tetrahydrothieno[3,2-c]pyridin-2-yl)-1-cyclopropyl-8-methoxy-4-oxo-1,4-dihydro-3-quinolinecarboxylic acid

The desired product was prepared by substituting Example 208B for Example 59C in Example 59D and was purified by trituration in methanol/Et₂O and filtered to give the solid product. MS (DCI/NH₃) m/z 412 (M+H)⁺; ¹H-NMR (300 MHz, d₆-DMSO) δ 10.25 (br s, 1H), 9.07 (br s, 2H), 8.82 (s, 1H), 8.18 (d, 1H), 8.04 (d, 1H), 7.78 (s, 1H), 5.00 (m, 1H), 4.45-4.20 (m, 3H), 3.80-3.60 (m, 5H), 1.20-1.00 (m, 4H).

### EXAMPLE 209

### 1-cyclopropyl-7-(4-hydroxy-4,5,6,7-tetrahydrothieno[2,3-c]pyridin-2-yl)-8-methoxcy-4-oxo-1,4-dihydro-3-quinolinecarboxylic acid

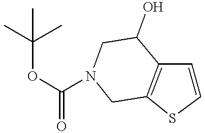

### EXAMPLE 209A

The desired product was prepared by substituting 4-hydroxy-4,5,6,7-tetrahydrothieno(2,3-c)pyridine was (prepared by the method of Maffrand, et.al.; Heterocycles; 1980, 14; 321-324) for 7-hydroxy-4,5,6,7-tetrahydrothieno(3,2-c)pyridine in Example 202A.

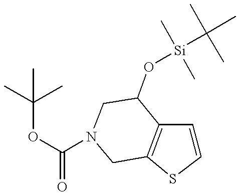

### EXAMPLE 209B

The desired product was prepared by substituting Example 209A for Example 202A in Example 202B.

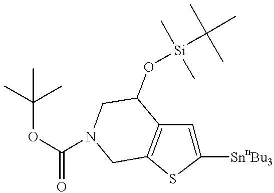

### EXAMPLE 209C

The desired product was prepared by substituting Example 209B for Example 202B in Example 202C.

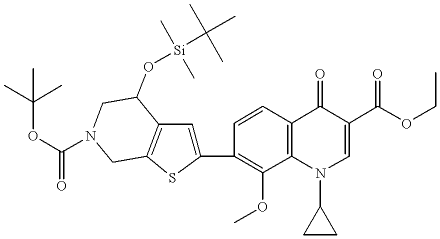

### EXAMPLE 209D

The desired product was prepared by substituting Example 209C and ethyl 7-bromo-1-cyclopropyl-8-methoxy-4-oxo-1,4-dihydro-3-quinolinecarboxylate for Example 1D and 7-bromo-1-cyclopropyl-4-oxo- 1,4-dihydro-3 -quinolinecarboxylate, respectively in Example 1E.

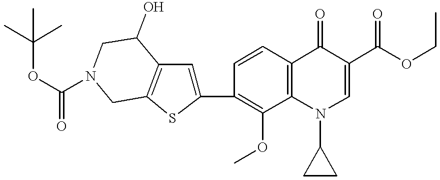

### EXAMPLE 209E

The desired product was prepared by substituting Example 209D for Example 35D in Example 35E.

### EXAMPLE 209F

### 1-cyclopropyl-7-(4-hydroxy-4,5,6,7-tetrahydrothieno[2,3-c]pyridin-2-yl)-8-methoxy-4-oxo-1,4-dihydro-3-quinolinecarboxylic acid

The desired product was prepared by substituting Example 209E for Example 59C in Example 59D. MS (DCI/NH₃) m/z 413 (M+H)⁺; ¹H-NMR (300 MHz, d₆-DMSO) δ 9.90 (br s, 1H), 9.42 (br s, 1H), 8.80 (s, 1H), 8.15 (d, 1H), 8.03 (d, 1H), 7.83 (s, 1H), 4.92 (t, 1H), 4.55-4.30 (m, 2H), 4.25 (m,1H), 3.68 (s, 3H), 3.40 (m, 2H), 1.15 (m, 2H), 1.05 (m,2H).

### EXAMPLE 210

### 1-cyclopropyl-6-fluoro-8-methoxy-7-(4-(methylamino)-4,5,6,7-tetrahydro-1-benzothien-2-yl)-4-oxo-1,4-dihydro-3-quinolinecarboxylic acid hydrochloride

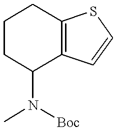

### EXAMPLE 210A

The desired product was prepared by substituting 4-keto4,5,6,7-tetrahydrothianaphthene for 7-keto4,5,6,7-tetrahydrothianaphthene in Example 216A.

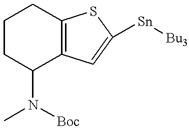

### EXAMPLE 210B

The desired product was prepared by substituting Example 210A for Example 218B in Example 218C.

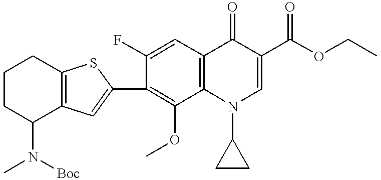

### EXAMPLE 210C

The desired product was prepared by substituting Example 210B, Example 374G, and a reaction time of 3 hours for Example ID and ethyl 7-bromo-1-cyclopropyl-4-oxo-1,4-dihydro-3-quinolinecarboxylate and a reaction time of 24 hours in Example aand was purified by chromatography on silica gel with 1% HOAc in ethyl acetate.

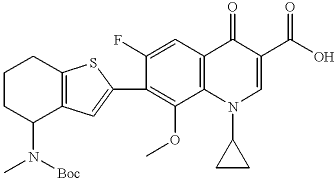

### EXAMPLE 210D

The desired product was prepared by substituting Example 210C for Example 2A in Example 2B.

### EXAMPLE 210E

### 1-cyclopropyl-6-fluoro-8-methoxy-7-(4-(methylamino)-4,5,6,7-tetrahydro-1-benzothien-2-yl)-4-oxo-1,4-dihydro-3-quinolinecarboxylic acid hydrochloride

The desired product was prepared by substituting Example 210D for Example 40B in Example 40C. MS (ESI) m/z 443 (M+H )⁺¹H NMR (300 MHz, DMSO-d₆) δ 9.10 (br,1H),7.96 ( d,1H),7.85 (d,1H),4.43 (m, 1H),4.22(m, 1H),4.02(s,1H),3.65(s,3H),2.90(m,2H),262(s,3H),2.15-1.97 (m,3H), 1.86 (m, 1H),1.15 (m,2H), 1.10 (m,2H).

### EXAMPLE 211

### 1-cyclopropyl-8-methoxy-4-oxo-7-(4-oxo-4,5,6,7-tetrahydrothieno[2,3-c]pyridin-2-yl)-1,4-dihydro-3-quinolinecarboxylic acid hydrochloride

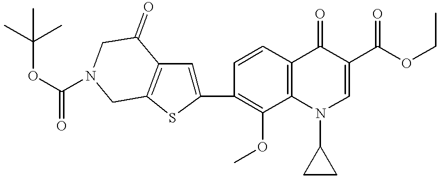

### EXAMPLE 211A

The desired product was prepared by substituting Example 209E for Example 41C in Example 324511A and was purified by silica gel chromatography eluting with a gradient from 20-30% acetone in hexanes.

### EXAMPLE 211B

### 1-cyclopropyl-8-methoxy-4-oxo-7-(4-oxo-4,5,6,7-tetrahydrothieno[2,3-c]pyridin-2-yl)-1,4-dihydro-3-quinolinecarboxylic acid hydrochloride

The desired product was prepared by substituting Example 21 1A for Example 59C in Example 59D. MS (DCI/NH₃) m/z 411 (M+H)⁺; ¹H-NMR (300 MHz, d₆-DMSO) δ 10.30 (br s, 1H), 8.82 (s, 1H), 8.20 (d, 1H), 8.17 (s, 1H), 8.16 (d, 1H), 4.77 (s, 2H), 4.26 (m, 1H), 4.06 (s, 2H), 3.71 (s, 3H), 1.15 (m, 2H), 1.05 (m, 2H).

### EXAMPLE 212

### 7-[4-(3-amino-1-azetidinyl)-4,5,6,7-tetrahydro-1-benzothien-2-yl]-1-cyclopropyl-8-methoxy-4-oxo-1,4-dihydro-3-quinolinecarboxylic acid dihydrochloride

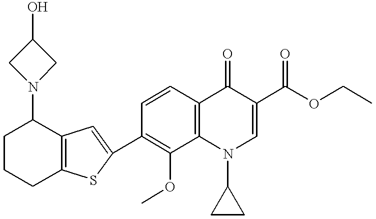

### EXAMPLE 212A

A solution of Example 83A (1.26 g, 2.9 mmol) in isopropanol (25 mL) was treated with epichlorohydrin (0.27 mL, 3.7 mmol) at 75° C. for 65 hours. The reaction mixture was partitioned between water and dichloromethane, dried (Na₂SO₄), and the concentrated residue purified by silica gel chromatography eluting with a gradient of 1 % to 6% methanol in dichloromethane to yield (0.34 g, 26%) of the desired compound.

### EXAMPLE 212B

The desired product was prepared by substituting Example 212A for Example 358696E in Example 358696F and was used without further purification.

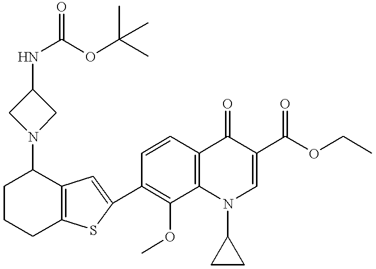

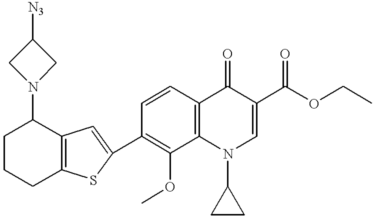

### EXAMPLE 212C

The desired product was prepared by substituting Example 212B for Example 201C in Example 201D and the crude product purified by silica gel chromatography eluting with a gradient of 1% to 3% methanol in dichloromethane to.

Example 212D

### 7-[4-(3-amino-1-azetidinyl)-4,5,6,7-tetrahydro-1-benzothien-2-yl]-1-cyclopropyl-8-methoxy-4-oxo-1,4-dihydro-3-quinolinecarboxylic acid dihydrochloride

The desired product was prepared by substituting Example 212C and tetrahydrofuran as the reation solvent for Example 48C and ethanol as the reaction solvent in Example 48D. MS (ESI+) m/z 466 (M+H)⁺; ¹H NMR (300 MHz, CD₃OD) δ 8.97 (s, 1H), 8.23 (d, 1H), 7.99 (d, 1H), 7.83 (s, 1H), 4.48-4.74 (m, 4H), 4.37 (m, 1H), 4.28 (m, 1H), 3.72 (s, 3H), 2.86-3.14 (m, 2H), 2.00-2.20 (m, 4H), 1.28 (m, 2H), 1.06 (m, 2H).

### EXAMPLE 213

### 1-cyclopropyl-7-(4-hydroxy-5,5-dimethyl-4,5,6,7-tetrahydro-1-benzothien-2-yl)-8-methoxy-4-oxo-1,4-dihydro-3-quinolinecarboxylic acid

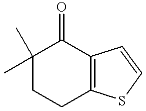

### EXAMPLE 213A

An ovennied system under positive N₂ atmosphere was charged with 60% NaH/mineral oil (2.20 g, 56.2 mmol), which was washed twice with hexane to remove the mineral oil. Added 40 mL dry DMF and cooled in an ice bath. To this was added 4-keto-4,5,6,7-tetrahydrothianaphthene (3.37 g, 22.0 mmol) in 40 mL of dry DMF dropwise with stirring. After 30 minutes Mel (5.75 mL, 92.5 mmol) was added dropwise with stirring. After 30 minutes the reaction was warmed to 25° C. and stirred for 22 hours. The reaction was quenched by pouring into 250 mL 10% NH₄Cl. Extracted twice with CU₂Cl₂. The combined organic layers were washed five times with water, dried (Na₂SO₄), filtered and concentrated. The entire procedure was repeated a second time to give complete dialkylation. The product was purified by silica gel chromatography eluting with CH₂Cl₂ to give the desired product as a colorless oil (3.22 g, 82%).

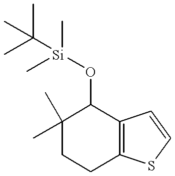

### EXAMPLE 213B

The desired product was prepared by substituting Example 213A for Example 218A in Example 218B.

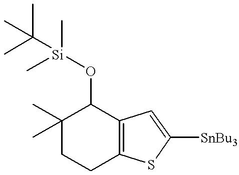

### EXAMPLE 213C

The desired product was prepared by substituting Example 213B for Example 218B in Example 218C.

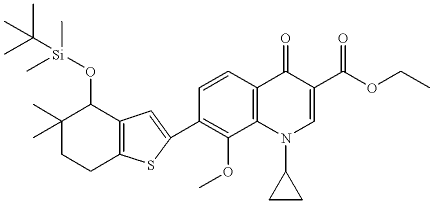

### EXAMPLE 213D

The desired product was prepared by substituting Example 213C and ethyl 7-bromo-1-cyclopropyl-8-methoxy-4-oxo-1,4-dihydro-3-quinolinecarboxylate and a reaction time of 6 hours for Example 1D and 7-bromo-1-cyclopropyl-4-oxo-1,4-dihydro-3-quinolinecarboxylate and a reaction time of 24 hours, respectively in Example 1E

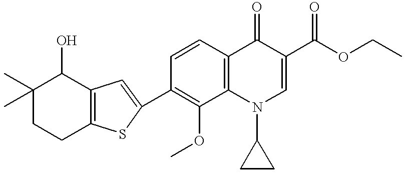

### EXAMPLE 213E

The desired product was prepared by substituting Example 213D for Example 35D in Example 35E.

### EXAMPLE 213F

### 1-cyclopropyl-7-(4-hydroxy-5,5-dimethyl-4,5,6,7-tetrahydro-1-benzothien-2-yl)-8-methoxy-4-oxo-1,4-dihydro-3-quinolinecarboxylic acid

The desired product was prepared by substituting Example 213E for Example 2A in Example 2B. mp 93-94° C.; MS (APCI) m/z 440 (M+H)⁺; ¹H NMR (300 MHz, DMSO-d₆) δ 14.94 (br s, 1H), 8.78 (s, 1H), 8.12 (d, 1H), 7.95 (d, 1H), 7.70 (s, 1H), 5.10 (d, 1H), 4.25 (m, 1H), 4.18 (d, 1H), 3.70 (s, 3H), 2.70 (m, 2H), 1.60 (m, 2H), 1.18 (m, 2H), 1.07 (m, 2H), 0.95 (s, 3H), 0.90 (s, 3H).

### EXAMPLE 216

### 1-cyclopropyl-8-methoxy-7-(7-(methylamino)-4,5,6,7-tetrahydro-1-benzothien-2-yl)-4-oxo-1,4-dihydro-3-quinolinecarboxylic acid hydrochloride

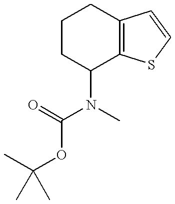

### EXAMPLE 216A

A solution of 7-keto-4,5,6,7-tetrahydrothienanapthene prepared by the method of Caubere, et al. Eur. J. Med. Chem. 1998, 867-77) (1.819g, 12.0 mmol) and N-methyl ammonium acetate (14.32 g, 157 mmol) in methanol (25 ntL) was cooled to 0+ C. and treated with sodium cyanoborohydride (0.530 g, 8.40 mmol). The mixture was allowed to warm to room temperature and stirred for seven days. Water (5 mL) was added, the mixture was partitioned between 10% HCl and ethyl acetate. The aqueous phase was made basic with 1 N NaOH and extracted with ethyl acetate. The organic phase was washed with water and brine, dried (Na₂SO₄), and concentrated. The resulting crude amine (1.081 g, 6.50 mmol) was dissolved in CH₂Cl₂ (15 mL) and treated with di-tert-butyl dicarbonate (1.53 g, 7.01 mmol) and stirred overnight. The reaction mixture was diluted with CH₂Cl₂ and washed with water and brine, dried (Na₂SO₄), and concentrated. The residue was purified by silica gel chromatography eluting with a gradient of hexane to 25% ethyl acetate in hexane to provide the desired product (1.735 g, 54%) as an orange oil.

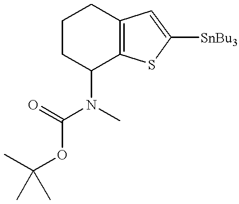

### EXAMPLE 216B

The desired product was prepared by substituting Example 216A for Example 218B in Example 218C.

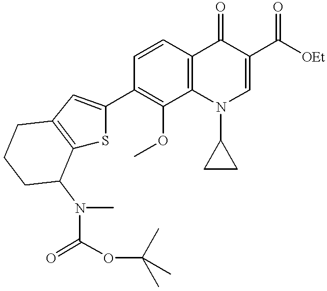

### EXAMPLE 216C

The desired product was prepared by substituting Example 216B and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E.

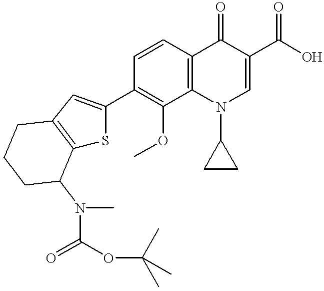

### EXAMPLE 216D

The desired product was prepared by substituting Example 216C for Example 2A in Example 2B.

### EXAMPLE 216E

### 1-cyclopropyl-8-methoxy-7-(7-(methylamino)-4,5,6,7-tetrahydro-1-benzothien-2-yl)-4-oxo-1,4-dihydro-3-quinolinecarboxylic acid hydrochloride

The desired product was prepared by substituting Example 216D for Example 40B in Example 40C. MS (DCI/NH3) m/z 425 (M+1)⁺; ¹H NMR (300 MHz, DMSO-d₆) δ 8.82 (s, 1H), 8.16 (d, 1H), 8.05 (d, 1H), 7.71 (s, 1H), 4.58 (m, 1H), 4.29 (m, 1H), 3.71 (s, 3H), 2.87-2.67 (m, 5H), 2.20-1.75 (m, 4H), 1.17-1.05 (m, 4H).

### EXAMPLE 218

### 1-cyclopropyl-7-(7-hydroxy-4,4-dimethyl-4,5,6,7-tetrahydro-1-benzothien-2-yl)-8-methoxy-4-oxo- 1,4-dihydro-3 -quinolinecarboxylic acid

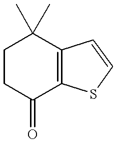

### EXAMPLE 218A

A solution 4,4-dimethyl-4,5,6,7-tetrahydrobenzo[b]thiophene (7.7 g, 46.3 mmol, prepared by the method of Reetz, et. al. Chem. Ber. 1985, 118, 1050-1057) in 250 mL of 1:1 acetic acid/water at −15° C. was treated dropwise with a solution of Ce(SO₄)₂ (74 g, 400 mmol) dissolved in 400 mL of water. The mixture was stirred for 16 hours, diluted with 500 mL of water and extracted 3×200 mL with Et₂O. The extracts were combined, washed consecutively with 1N NaOH, water, brine, dried (MgSO₄), concentrated and the residue purified by flash chromatography on silica gel eluting with 10% EtOAc in hexane to give the desired compound (3.6 g, 43%) as a yellow oil.

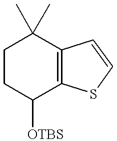

### EXAMPLE 218B

A solution of Example 218A (4.17 g, 23.2 mmol) in EtOH (75 ml) was treated with NaBH₄ (880 mg, 23.2 mmol), heated at 50° C. for 2 hours, cooled, poured into 200 mL of iced water and extracted with EtOAc. The organic phase was washed with brine, dried (MgSO₄) and concentrated to give the crude alcohol which was used without purification.

A solution of the crude alcohol (4.2 g, 23.2 mmol) and imidazole (1.9 g, 28 mmol) in methylene chloride (50 ml) at 0° C. was treated with tert-butyldimethylchlorosilane (3.85 g, 25.5 mmol), warmed to ambient temperature, stirred for 16 hours and partitioned between water and dichloromethane. The dichloromethane layer was separated, washed with brine, dried (MgSO₄) and concentrated. The resulting residue was purified by flash chromatography on silica gel eluting with 10% EtOAc in hexane to give the desired compound (6.3 g, 92%) as a yellow oil.

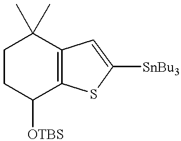

### EXAMPLE 218C

A solution of diisopropyl amine (3.6 mL, 25.5 mmol) in THF (50 mL) was cooled to 0° C., treated dropwise with n-BuLi (11.1 mL of a 2.5 M solution in hexanes, 27.7 mmol), stirred for 30 minutes and cooled to −50° C. This solution was treated dropwise with a solution of Example 218B (6.3 g, 21.3 mmol) in THF (10 mL), stirred for 1 hour at 0° C., recooled to −50° C., treated with chlorotributylstannane (9.0 g, 27.7 mmol) and allowed to warm to room temperature overnight. The reaction mixture was partitioned between water and ethyl acetate, the aqueous phase extracted with ethyl acetate, the combined organic phases washed with water, brine, dried (Na₂SO₄) and concentrated to provide the desired product that was used without further purification.

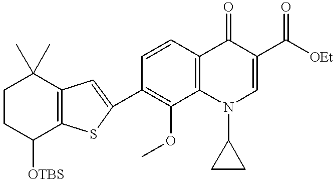

### EXAMPLE 218D

The desired product was prepared by substituting Example 218C and ethyl 1-cyclopropyl-7-bromo-8-methoxy-4-oxo- 1,4-dihydroquinoline-3 -carboxylate for Example 1D and ethyl 1-cyclopropyl-7-bromo-4-oxo-1,4-dihydroquinoline-3-carboxylate, respectively in Example 1E and purified by silica gel chromatography eluting with 2:1 hexane:acetone.

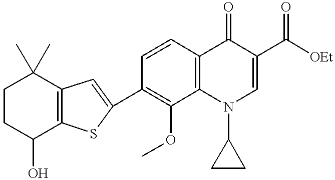

### EXAMPLE 218E

The desired product was prepared by substituting Example 218D for Example 35D in Example 35E and was used without further purification.

### EXAMPLE 218F

### 1-cyclopropyl-7-(7-hydroxy-4,4-dimethyl-4,5,6,7-tetrahydro-1-benzothien-2-yl)-8-methoxy-4-oxo-1,4-dihydro-3-quinolinecarboxylic acid

The desired product was prepared by substituting Example 218E for Example 2A in Example 2B and the crude product purified by chromatography on silica gel eluting with 97:3 methylene chloride/methanol. MS (APCI) m/z 440 (M+H)⁺; ¹H NMR (300 MHz, DMSO- d₆) δ 14.95 (s, 1H), 8.79 (s, 1H), 8.10 (d, 1H), 8.07 (d, 1H), 7.76 (s, 1H), 5.51 (d, 1H), 4.71 (m, 1H), 4.26 (m, 1H), 3.69 (s, 3H), 2.01 (m, 1H), 1.81-1.55 (m, 3H), 1.27 (s, 3H), 1.26 (s, 3H), 1.15 (m, 2H), 1.03 (m, 2H).

### EXAMPLE 222

### 7-(7-amino-4,4-dimethyl-4,5,6,7-tetrahydro-1-benzothien-2-yl)-1-cyclopropyl-8-methoxy-4-oxo-1,4-dihydro-3-quinolinecarboxylic acid hydrochloride

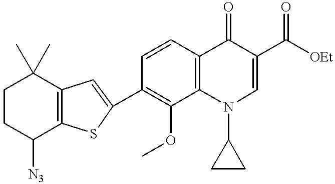

### EXAMPLE 222A

The desired product was prepared by substituting Example 218E for Example 37A in Example 37B and was purified by silica gel chromatography eluting with 2:1 hexane:acetone.

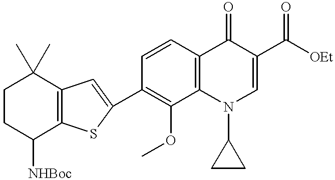

### EXAMPLE 222B

The desired product was prepared by substituting Example 222A for Example 201C in Example 201D and separating the components by silica gel chromatography eluting with 3:1 hexane:acetone. Example 227A was also isolated as a by-product.

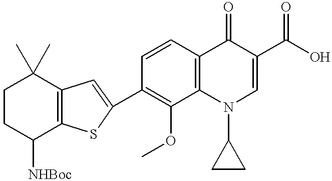

### EXAMPLE 222C

The desired product was prepared by substituting Example 222B for Example 2A in Example 2B.

### EXAMPLE 222D

### 7-(7-amino-4,4-dimethyl-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 222C for Example 40B in Example 40C. MS (APCI) m/z 439 (M+H)⁺; 1H NMR (300 MHz, DMSO-d₆) δ 8.82 (s, 1H), 8.52 (bs, 3H), 8.16 (d, 1H), 8.13 (d, 1H), 7.91 (s, 1H), 4.56 (m, 1H), 4.27 (m, 1H), 3.72 (s, 3H), 2.20-1.60 (m, 4H), 1.35 (s, 3H), 1.26 (s, 3H), 1.15-1.00 (m, 4H).

### EXAMPLE 224

### 7-(4-amino-4,5,6,7-tetrahydro-1-benzothien-2-yl)-1-cyclopropyl-6-fluoro-8-methoxy-4-oxo-1,4-dihydro-3-quinolinecarboxylic acid hydrochloride

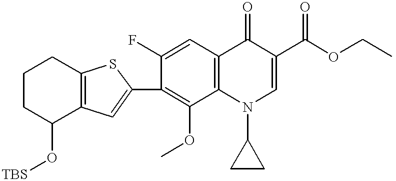

### EXAMPLE 224A

The desired product was prepared by substituting Example 38B and Example 374G for Example 1D and ethyl-7-bromo-1-cyclopropyl-4-oxo-1,4-dihydro-3-quinolinecarboxylate 1E and was purified by silica gel chromatography with 1% HOAc in ethyl acetate.

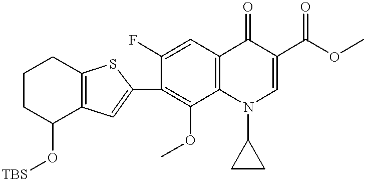

### EXAMPLE 224B

The desired product was prepared by substituting Example 224A for Example 38C in Example 39A.

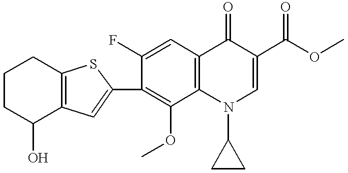

### EXAMPLE 224C

The desired product was prepared by substituting Example 224B for Example 35D in Example 35E.

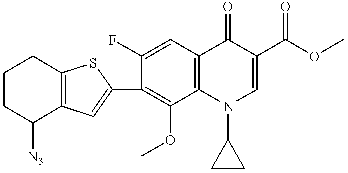

### EXAMPLE 224D

The desired product was prepared by substituting Example 224C for Example 37A in Example 37B.

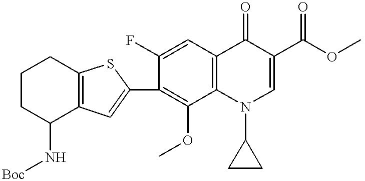

### EXAMPLE 224E

The desired product was prepared by substituting Example 224D for Example 201C in Example 201D.

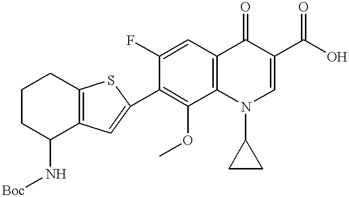

### EXAMPLE 224F

The desired product was prepared by substituting Example 224E for Example 2A in Example 2B.

### EXAMPLE 224G

### 7-(4-amino-4,5,6,7-tetrahydro-1-benzothien-2-yl)-1-cyclopropyl-6-fluoro-8-methoxv-4-oxo-1,4-dihydro-3-quinolinecarboxylic acid hydrochloride

The desired product was prepared by substituting Example 224F for Example 40B in Example 40C. MS(ESI) m/z 429 (M+H)⁺; ¹H NMR (300MHz,DMSO-d₆)-δ 8.80(s, 1H),8.53(br,2H),7.95(d, 1H),7.87(d, 1H),4.43(m, 1H),4.23(m,1H),3.66(s,3H),2.86(m,2H), 2.20-2.00(m,2H),1.87(m,2H),1.17(m,2H), 1 .08(m,2H).

### EXAMPLE 225

### 7-(4-amino-4,5,6,7-tetrahydrothieno[2,3 -c]pyridin-2-yl)-1-cyclopropyl-8-methoxy-4-oxo-1,4-dihydro-3-quinolinecarboxylic acid

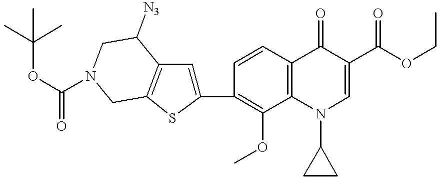

### EXAMPLE 225A

The desired product was prepared by substituting Example 209E for Example 37A in Example 37B and was purified by silica gel chromatography eluting with 20% acetone in hexanes.

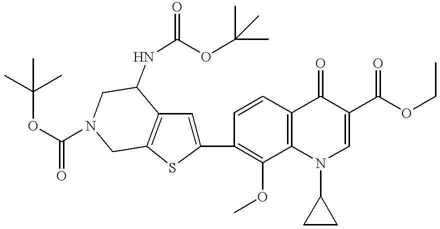

### EXAMPLE 225B

The desired product was prepared by substituting Example 225A for Example 353365C in Example 353365D and was purified by silica gel chromatography eluting with 20% acetone in hexanes.

### EXAMPLE 225C

### 7-(4-amino-4,5,6,7-tetrahydrothieno[2,3-c]pyridin-2-yl)-1-cyclopropyl-8-methoxy-4-oxo-1,4-dihydro-3-quinolinecarboxylic acid

The desired product was prepared by substituting Example 225B for Example 48C in Example 48D and the crude solid product was triturated in methanol/Et₂O, filtered and dried under vacuum. MS (DCI/NH₃) m/z 412 (M+H)⁺; ¹H-NMR (300 MHz, d₆-DMSO) δ 10.30 (br s, 1H), 9.10 (br s, 2H), 8.80 (s, 1H), 8.20 (d, 1H), 8.15 (s, 1H), 7.90 (d, 1H), 4.80 (m, 1H), 4.52 (dd, 2H), 4.27 (m, 1H), 3.72 (s, 3H), 3.80-3.58 (m, 1H), 3.40 (br m, 3H), 1.15 (m, 2H), 1.05 (m, 2H).

### EXAMPLE 226

### 7-(7-azido-4,5,6,7-tetrahydrothieno[3,2-c]pyridin-2-yl)-1-cyclopropyl-8-methoxy-4-oxo-1,4-dihydro-3-quinolinecarboxylic acid

The desired product was prepared by substituting Example 208A for Example 59C in Example 59D. MS (DCI/NH₃) m/z 438 (M+H)⁺; ¹H-NMR (300 MHz, d₆-DMSO) δ 10.20 (br s, 1H), 9.50 (br s, 1H), 8.80 (s, 1H), 8.18 (d, 1H), 8.02 (d, 1H), 7.78 (s, 1H), 5.48 (dd, 1H), 4.25 (m, 3H), 3.72 (s, 3H), 3.72-3.50 (m, 2H), 1.17 (m, 2H), 1.07 (m, 2H).

### EXAMPLE 227

### 1-cyclopropyl-7-(4,4-dimethyl-4,5,6,7-tetrahydro-1-benzothien-2-yl)-8-methoxy-4-oxo-1,4-dihydro-3-quinolinecarboxylic acid

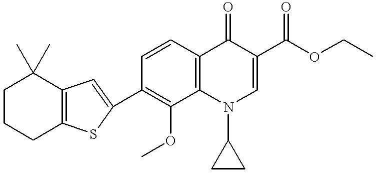

### EXAMPLE 227A

The desired product was obtained as a by-product from Example 222B and was separated by silica gel chromatography eluting with 3:1 hexane:acetone.

### EXAMPLE 227B

### 1-cyclopropyl-7-(4,4-dimethyl-4,5,6,7-tetrahydro-1-benzothien-2-yl)-8-methoxy-4-oxo-1,4-dihydro-3-quinolinecarboxylic acid

The desired product was prepared by substituting Example 227A for Example 2A in Example 2B and was used without further purification. MS (APCI) m/z 424 (M+H)⁺; ¹H NMR (300 MHz, DMSO- d₆) δ 14.96 (s, 1H), 8.79 (s, 1H), 8.10 (d, 1H), 8.08 (d, 1H), 7.81 (s, 1H), 4.25 (m, 1H), 3.68 (s, 3H), 2.76 (t, 2H), 1.85 (m, 2H), 1.61 (m, 2H), 1.27 (s, 6H), 1.14 (m, 2H), 1.02 (m, 2H).

### EXAMPLE 228

### 7-(7-amino-4,5,6,7-tetrahydro-1-benzothien-2-yl)-1-cyclopropyl-6-fluoro-8-methoxy-4-oxo-1,4-dihydro-3-quinolinecarboxylic acid hydrochloride

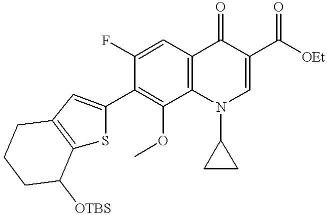

### EXAMPLE 228A

The desired product was prepared by substituting Example 41B and ethyl 7-bromo-1-cyclopropyl-6-fluoro-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E.

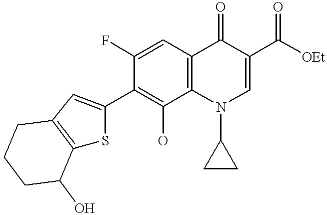

### EXAMPLE 228B

The desired product was prepared by substituting Example 228A for Example 35D in Example 35E and was purified by silica gel chromatography eluting with hexane then 50% acetone in hexane to provide the desired product.

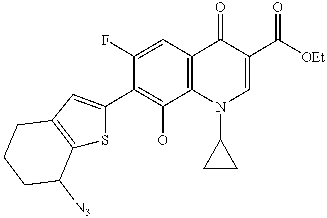

### EXAMPLE 228C

The desired product was prepared by substituting Example 228B for Example 37A in Example 37B.

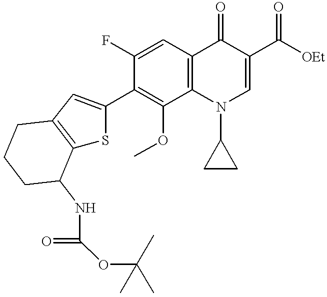

### EXAMPLE 228D

The desired product was prepared by substituting Example 228C for Example 201C in Example 201D.

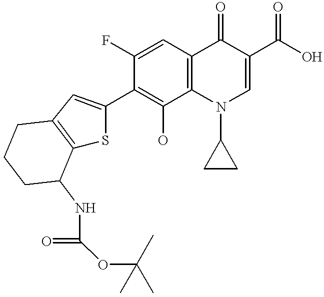

### EXAMPLE 228E

The desired product was prepared by substituting Example 228D for Example 2A in Example 2B.

### EXAMPLE 228F

### 7-(7-amino-4,5,6,7-tetrahydro-1-benzothien-2-yl)-1-cyclopropyl-6-fluoro-8-methoxy-4-oxo-1,4-dihydro-3-quinolinecarboxylic acid hydrochloride

The desired product was prepared by substituting Example 228E for Example 40B in Example 40C. Mp 148-153° C. (dec.); MS (APCI) m/z 429 (M+1)⁺; ¹H NMR (300 MHz, DMSO-d₆) δ 8.81 (s, 1H), 8.59 (br s, 2H), 7.95 (d, 1H), 7.42 (d, 1H), 4.63 (m, 1H), 4.26 (m, 1H), 3.57 (s, 3H), 2.80-2.63 (m, 2H), 2.16-1.82 (m, 4H), 1.20-1.06 (m, 4H); ¹³C NMR (75 MHz, DMSO-d₆) δ 176.3 (d), 165.5, 156.7 (d), 151.5, 150.2 (d), 139.5, 134.6, 133.1, 131.28 (d), 128.9, 126.9 (d), 123.1 (d), 107.2, 106.5 (d), 66.4, 63.0, 45.1, 27.9, 24.8, 18.3, 9.1, 9.0; IR (mic) 3420 (COOH), 1740 (C═O), cm⁻¹; Anal. calcd for C₂₂H₂₂N₂O₄SFCl.0.25 H₂O: C, 56.28; H, 4.83; N, 5.96; found: C, 56.29; H, 4.55; N, 5.62.

### EXAMPLE 229

### 7-(5-acetyl-7-amino-4,5,6,7-tetrahydrothieno[3,2-c]pyridin-2-yl)-1-cyclopropyl-8-methoxy-4-oxo-1,4-dihydro-3-quinolinecarboxylic acid

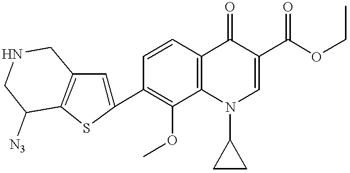

### EXAMPLE 229A

To a solution of 208A (0.100 mg, 0.177 mmol) in CH₂Cl₂ (2 mL) was added TFA (1 mL) at room temperature. After 30 minutes the reaction mixture was concentrated in vacuo. and triturated with Et₂O. The mixture was filtered, the solid washed with Et2O and dried under high vacuum to give the desired product ( 0.078 g, 95% yield).

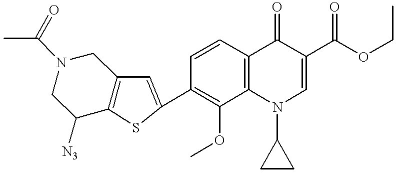

### EXAMPLE 229B

To a solution of Example 229A (250 mg, 0.537 mmol) in CH₂Cl₂ was added acetic anhydride (101 uL, 1.074 mmol) and triethyl amine (262 uL, 1.88 mmol). The reaction mixture was stirred overnight at room temperature, diluted with CH₂Cl₂ and washed sequentially with 1N HCl, sat. NaHCO₃, brine, dried (Na₂SO₄), and concentrated to give the desired product (0.264 g, 97% yield) that was used without further purification.

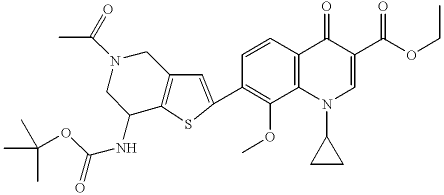

### EXAMPLE 229C

The desired product was prepared by substituting Example 229B for Example 201C in Example 201D.

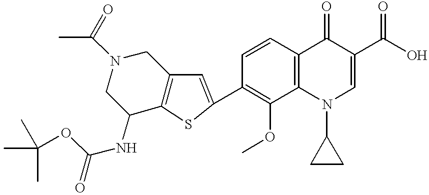

### EXAMPLE 229C

The desired product was prepared by substituting Example 229B for Example 2A in Example 2B.

### EXAMPLE 229D

### 7-(5-acetyl-7-amino-4,5,6,7-tetrahydrothieno[3,2-c]pyridin-2-yl)-1-cyclopropyl-8-methoxy-4-oxo-1,4-dihydro-3-quinolinecarboxylic acid

The desired product was prepared by substituting Example 229C for Example 59C in Example 59D. MS (DCI/NH₃) m/z 454 (M+H)⁺; ¹H-NMR (500 MHz, 900, d₆-DMSO) δ 8.80 (s, 1H), 8.60 (br s, 2H), 8.18 (d, 1H), 7.90 (d, 1H), 7.65 (s, 1H), 4.95 (m, 2H), 4.68 (m, 2H), 4.43 (m, 2H), 4.28 (m, 1H), 3.71 (s, 3H), 2.16 (s, 3H), 1.16 (m, 2H), 1.07 (m, 2H).

### EXAMPLE 230

### 7-(7-amino-5-(methylsulfonyl)-4,5,6,7-tetrahydrothieno[3,2-c]pyridin-2-yl)-1-cgclopropyl-8-methoxy-4-oxo-1,4-dihydro-3-quinolinecarboxylic acid

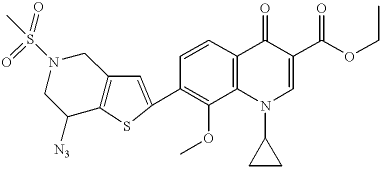

### EXAMPLE 230A

A solution of Example 229A (250 mg, 0.537 mmol) in CH₂Cl₂ (10 ml) was treated with triethylamine (224 uL, 1.611 mmol) and methanesulfonyl chloride (62 uL, 0.806 mmol) and the resulting mixture stirred overnight at ambient temperature. The reaction mixture was partitioned between CH₂Cl₂ and 1N HCl, the organic layer washed with sat. NaHCO₃, brine and dried (Na₂SO₄). The concentrated residue product was purified by silica gel chromatography eluting with 2% MeOH in CH₂Cl₂ to give the desired product (0.277 g, 95% yield).

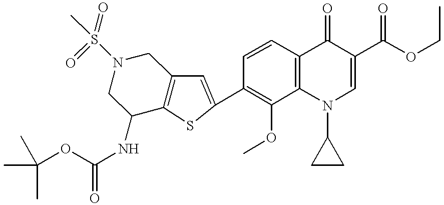

### EXAMPLE 230B

The desired product was prepared by substituting Example 230A for Example 353365C in Example 353365D.

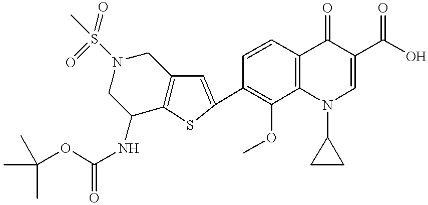

### EXAMPLE 230C

The desired product was prepared by substituting Example 230B for Example 2A in Example 2B.

### EXAMPLE 230D

### 7-(7-amino-5-(methylsulfonyl)-4,5,6,7-tetrahydrothieno[3,2-c]pyridin-2-yl)-1-cyclopropyl-8-methoxy-4-oxo-1,4-dihydro-3-quinolinecarboxylic acid

The desired product was prepared by substituting Example 230C for Example 40B in Example 40C. MS (DCI/NH₃) m/z 490 (M+H)⁺; ¹H-NMR (300 MHz, d₆-DMSO) δ 8.82 (s, 1H), 8.70 (br s, 2H), 8.17 (d, 1H), 8.04 (d, 1H), 7.79 (s, 1H), 4.80 (m, 1H), 4.57 (d, 1H), 4.29 (m, 2H), 3.95 (dd, 1H), 3.72 (s, 3H), 3.60-3.45 (m, 3H), 3.10 (s, 3H), 1.20-1.00 (m, 4H).

### EXAMPLE 232

### 7-(4-amino-3-methyl-4,5,6,7-tetrahydro-1-benzothien-2-yl)-1-cyclopropyl-8-methoxy-4-oxo-1,4-dihydro-3-quinolinecarboxylic acid hydrochloride

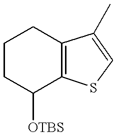

### EXAMPLE 232A

A solution of Example 260C (2.00 g, 5.78 mmol) in Et₂O (20 mL) was treated with methyl magnesium bromide (2 mL of a 3.0 M solution in diethyl ether, 6.00 mmol) and [1,3-bis(diphenylphosphino)-propane]dichloronickel (0.050 g, 0.10 mmol) and stirred at 35° C. overnight. The reaction mixture was diluted with ethyl acetate, washed with water and brine, dried (Na₂SO₄), and concentrated. The residue was purified by silica gel chromatography eluting with a gradient of hexane to 2% ethyl acetate in hexane to provide the desired product (0.701g, 43%) as an orange oil.

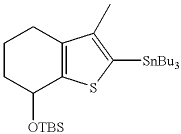

### EXAMPLE 232B

The desired product was prepared by substituting Example 232A for Example 218B in Example 218C.

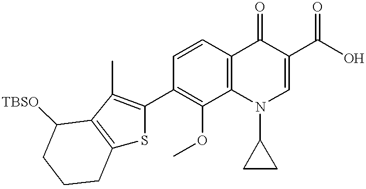

### EXAMPLE 232C

The desired product was prepared by substituting Example 232B and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E.

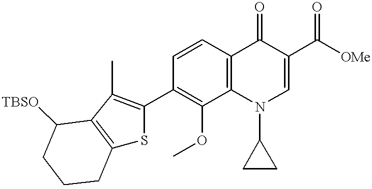

### EXAMPLE 232C

The desired product was prepared by substituting Example 232B for Example 38C in Example 39A.

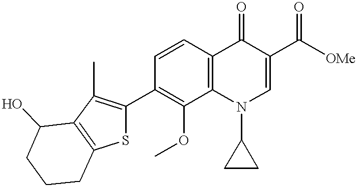

### EXAMPLE 232D

The desired product was prepared by substituting Example 232C for Example 35Din Example 35E and was purified by silica gel chromatography eluting with hexane then 50% acetone in hexane to provide the desired product.

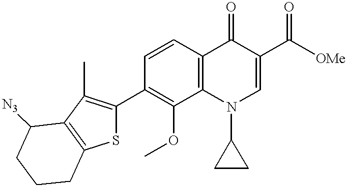

### EXAMPLE 232E

The desired product was prepared by substituting Example 232D for Example 37A in Example 37B and was purified by silica gel chromatography eluting with hexane then 50% acetone in hexane to provide the desired product.

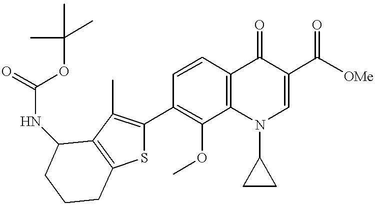

### EXAMPLE 232F

The desired product was prepared by substituting Example 232E for Example 201C in Example 201D.

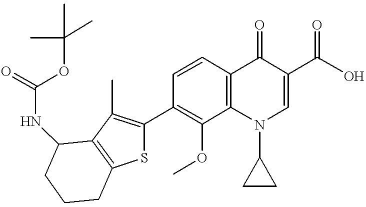

### EXAMPLE 232G

The desired product was prepared by substituting Example 232F for Example 2A in Example 2B.

### EXAMPLE 232H

### 7-(4-amino-3-methyl-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 232G for Example 40B in Example 40C. MS (APCI) m/z 408 (M-18+1)⁺; ¹H NMR (300 MHz, DMSO-d₆) δ 8.82 (s, 1H), 8.16 (m, 3H), 7.51 (d, 1H), 4.48 (m, 1H), 4.25 (m, 1H), 3.57, (s, 3H), 3.47 (s, 3H), 3.03-2.70 (m, 2H), 2.10-1.88 (m, 4H), 1.18-1.09 (m, 4H).

### EXAMPLE 233

### 1-cyclopropyl-7-((4E/Z)-4-(hydroxyimino)-5,5-dimethyl-4,5,6,7-tetrahydro-1-benzothien-2-yl)-8-methoxy-4-oxo-1,4-dihydro-3-quinolinecarboxylic acid

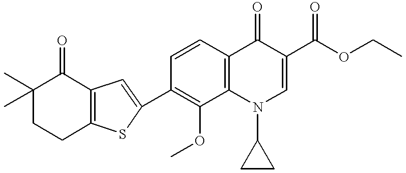

### EXAMPLE 233A

The desired product was prepared by substituting Example 213E for Example 41C in Example 61A.

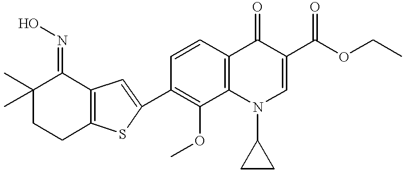

### EXAMPLE 233B

The desired product was prepared by substituting Example 233A, a reaction temperature of 60° C. and a reaction time of 48 hours for Example 50, a reaction temperature of 90° C. and a reaction time of 18 hours, respectively in Example 64.

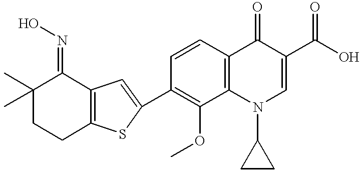

### EXAMPLE 233C

### 1-cyclopropyl-7-((4E/Z)-4-(hydroxyimino)-5,5-dimethyl-4,5,6,7-tetrahydro-1-benzothien-2-yl)-8-methoxy-4-oxo-1,4-dihydro-3-quinolinecarboxylic acid

The desired product was prepared by substituting Example 233B for Example 2A in Example 2B. mp 135-138° C.; MS (APCI) m/z 453 (M+H)⁺; ¹H NMR (300 MHz, CDCl₃) δ 8.90 (s, 1H), 8.61 (s, 1H), 8.25 (d, 1H), 7.82 (d, 1H), 4.10 (m, 1H), 3.68 (s, 3H), 3.03 (dd, 2H), 1.95 (dd, 2H), 1.60 (br s, 2H), 1.20 (m, 2H), 1.18 (s, 6H), 1.06 (m, 2H).

### EXAMPLE 234

### 1-cyclopropyl-7-(4-hydroxy-6,6-dimethyl-4,5,6,7-tetrahydro-1-benzothien-2-yl)-8-methoxy-4-oxo-1,4-dihydro-3-quinolinecarboxylic acid

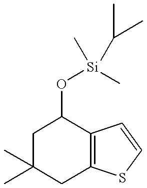

### EXAMPLE 234A

The desired product was prepared by substituting 6,7-dihydro-6,6-dimethylbenzo[b]thiophen-4-one (prepared by the method of Sen, et al. Indian J. Chem 1999, 38B, 648-656.) and a reaction temperature of 0° C. for Example 218A and a reaction temperature of 50° C. in Example 218B.

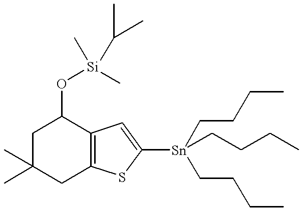

### EXAMPLE 234B

The desired product was prepared by substituting Example 234A for Example 218B in Example 218C.

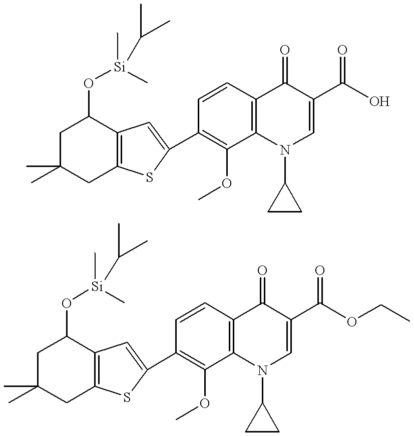

### EXAMPLE 234C EXAMPLE 238A

The desired products were prepared by substituting Example 234B, ethyl 7-bromo-1-cyclopropyl-8-methoxy-4-oxo-1,4-dihydro-3-quinolinecarboxylate and a reaction time of 4 hours for Example 1D, ethyl 7-bromo-1-cyclopropyl-4-oxo-1,4-dihydro-3-quinolinecarboxylate and a reaction time of 24 hours, respectively in Example 1E and the resulting mixture separated by silica gel chromatography. Example 238A was isolated as a by-product.

### EXAMPLE 234D

### 1-cyclopropyl-7-(4-hydroxy-6,6-dimethyl-4,5,6,7-tetrahydro-1-benzothien-2-yl)-8-methoxy-4-oxo-1,4-dihydro-3-quinolinecarboxylic acid

The desired product was prepared by substituting Example 234C for Example 35D in Example 35E and the crude product triturated in 1:1 diethyl ether: ethanol and filtered to give the desired solid. MS (DCI/NH₃) m/z 440 (M+H)⁺; ¹H NMR (300 MHz, CD₃OD) δ 8.94 (s, 1H), 8.20 (d, 1H), 7.99 (d, 1H), 7.74 (s, 1H), 4.76 (m, 1H), 4.28 (m, 1H), 3.70 (s, 3H), 2.65 (m, 2H), 1.95 (m, 1H), 1.58 (m, 1H), 1.27 (m, 2H0, 1.17 (s, 3H), 1.06 (m, 2H), 1.03 (s, 3H).

### EXAMPLE 235

### 7-(7-amino-6-methyl-4,5,6,7-tetrahydro-1-benzothien-2-yl)-1-cyclopropyl-8-methoxy-4-oxo-1,4-dihydro-3-quinolinecarboxylic acid hydrochloride

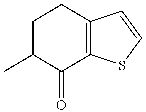

### EXAMPLE 235A

A solution of 7-oxo-4,5,6,7-tetrahydrobenzo[b]thiophene (8.0 g, 52.6 mmol, prepared by the method of Cagniant, et. al. Tet. Lett. 1975, 2885) in anhydrous THF (200 mL) at −78° C. was treated dropwise with lithium bis(trimethylsilyl)amide (58 mL of a 1M solution in hexanes, 58 mmol), stirred for 1 hour, treated with methyl iodide (22.4 g, 158 mmol), warmed to ambient temperature and partitoned between saturated ammonium chloride and EtOAc. The organic phase was washed with brine, dried (MgSO₄), concentrated and the residue purified by flash chromatography on silica gel eluting with 10% EtOAc in hexane to give the desired compound (5.9 g, 68%) as a colorless oil.

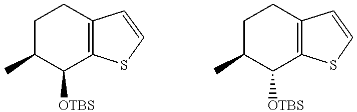

### EXAMPLE 242A EXAMPLE 235B

The desired product was prepared by substituting Example 23 5A for Example 218A in Example 218B and separating the diastereomers by silica gel chromatography eluting with hexanes. [Example 242A (cis) R_{f}=0.55; Example 235B (trans) R_{f}=0.42].

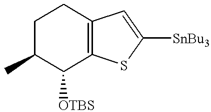

### EXAMPLE 235C

The desired product was prepared by substituting Example 235B for Example 218B in Example 218C and was used without fuither purification.

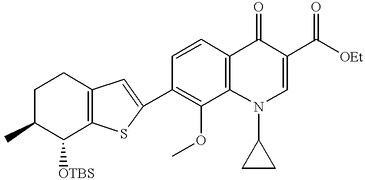

### EXAMPLE 235D

The desired product was prepared by substituting Example 235C, ethyl 1-cyclopropyl-7-bromo-8-methoxy-4-oxo-1,4-dihydroquinoline-3-carboxylate and a reation time of 5 hours for Example 1D, ethyl 1-cyclopropyl-7-bromo-4-oxo-1,4-dihydroquinoline-3-carboxylate and a reaction time of 24 hours, respectively in Example 1E and purifying by chromatography on silica eluting with 2:1 hexane/acetone.

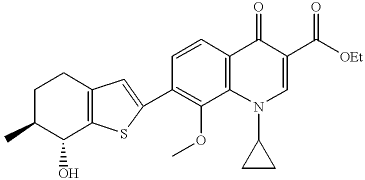

### EXAMPLE 235E

The desired product was prepared by substituting Example 235D for Example 35D in Example 35E and was use without further purification.

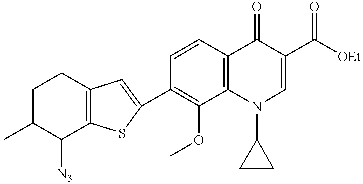

### EXAMPLE 235F

The desired product was prepared by substituting Example 235E for Example 37A in Example 37B and was purified by chromatography on silica eluting with 2:1 hexane/acetone to give an inseparable mixture of diastereomers.

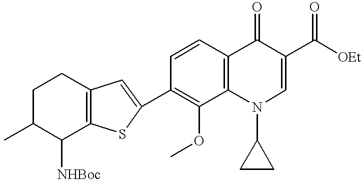

### EXAMPLE 235G

The desired product was prepared by substituting Example 235F for Example 201C in Example 201D.

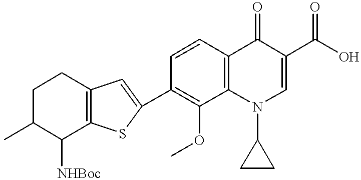

### EXAMPLE 235H

The desired product was prepared by substituting Example 235G for Example 2A in Example 2B.

### EXAMPLE 235I

### 7-(7-amino-6-methyl-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared as a 2:1 mixture (trans/cis) of diastereomers by substituting Example 235H for Example 40B in Example 40C. MS (APCI) m/z 425 (M+H)⁺; Major (trans): 1H NMR (300 MHz, DMSO-d₆) δ 8.82 (s, 1H), 8.49 (bs, 3H), 8.14 (d, 1H), 8.05 (d, 1H), 7.68 (s, 1H), 4.28 (m, 1H), 4.26 (m, 1H), 3.72 (s, 3H), 2.70 (t, 2H), 2.25 (m, 1H), 2.03 (m, 1H), 1.64 (m, 1H), 1.20-0.98 (m, 4H), 1.05 (s, 3H).

Minor (cis): 1H NMR (300 MHz, DMSO-d₆) δ 8.82 (s, 1H), 8.32 (bs, 3H), 8.14 (d, 1H), 8.05 (d, 1H), 7.67 (s, 1H), 4.57 (m, 1H), 4.26 (m, 1H), 3.72 (s, 3H), 2.67 (m, 2H), 2.22 (m, 1H), 1.90-1.72 (m, 2H), 1.20-0.98 (m, 4H), 1.10 (s, 3H).

### EXAMPLE 238

### 7-(4-amino-6,6-dimethyl-4,5,6,7-tetrahydro-1-benzothien-2-yl)-1-cyclopropyl-8-methoxy-4-oxo-1,4-dihydro-3-quinolinecarboxylic acid hydrochloride

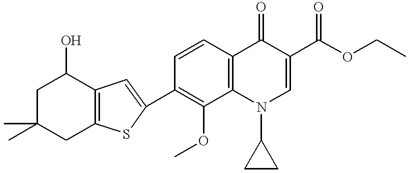

### EXAMPLE 238B

The desired product was prepared by substituting Example 238A in Example 234C for Example 35D in Example 35E and was used without purification.

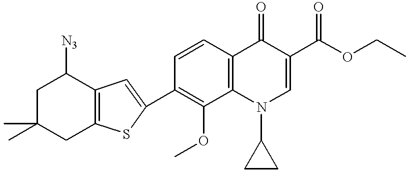

### EXAMPLE 238C

The desired product was prepared by substituting Example 238B and dichloromethane as reaction solvent for Example 37A and 1:1 toluene:dichloromethane as reaction solvent in Example 37B.

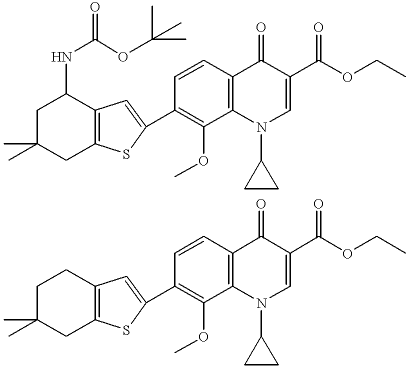

### EXAMPLE 238D EXAMPLE 236A

The desired products were prepared by substituting Example 238C for Example 201C in Example 201D and the resulting mixture separated by silica gel chromatography eluting with a gradient of 0% to 2% methanol in dichloromethane. Example 236A was isolated as a by-product.

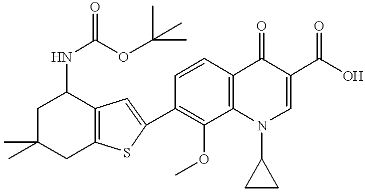

### EXAMPLE 238E

The desired products were prepared by substituting Example 238D for Example 2A in Example 2B.

### EXAMPLE 238F

### 7-(4-amino-6,6-dimethyl-4,5,6,7-tetrahydro-1-benzothien-2-yl)-1-cyclopropyl-8-methoxy-4-oxo-1,4-dihydro-3-quinolinecarboxylic acid hydrochloride

The desired products were prepared by substituting Example 238E for Example 40B in Example 40C. MS (DCI/NH₃) m/z 439 (M+H)⁺; ¹H NMR (300 MHz, CD₃OD) δ 8.98 (s, 1H), 8.24 (d, 1H), 7.90 (d, 1H), 7.82 (s, 1H), 4.49 (m, 1H), 4.29 (m, 1H), 3.72 (s, 3H), 2.77 (m, 2H), 2.09 (dd, 1H), 1.68 (dd, 1H), 1.28 (m, 2H), 1.24 (s, 3H), 1.08 (m, 2H), 1.05 (s, 3H).

### EXAMPLE 236

### 1-cyclopropyl-7-(6,6-dimethyl-4,5,6,7-tetrahydro-1-benzothien-2-yl)-8-methoxy-4-oxo-1,4-dihydro-3-quinolinecarboxylic acid

The desired products were prepared by substituting Example 236A in Example 238D for Example 40B in Example 40C. MS (DCI/NH₃) m/z 424 (M+H)⁺; ¹H NMR (300 MHz, CD₃0D) δ 8.94 (s, 1H), 8.28 (d, 1H), 7.89 (d, 1H), 7.47 (s, 1H), 4.28 (m, 1H), 3.70 (s, 3H), 2.72 (t, 2H), 2.61 (s, 2H), 1.64 (t, 2H) 1.27 (m, 2H), 1.06 (m, 2H), 1.04 (s, 6H).

### EXAMPLE 237

### 7-(7-amino-6-fluoro-4,5,6,7-tetrahydro-1-benzothien-2-yl)-1-cyclopropyl-8-methoxy-4-oxo-1,4-dihydro-3-quinolinecarboxylic acid hydrochloride

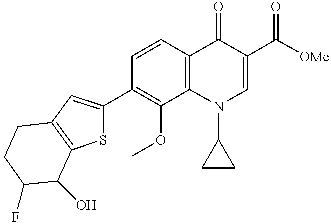

### EXAMPLE 237A

The desired product was prepared by substituting Example 240E for Example 35D in Example 35E and was purified by silica gel chromatography eluting with hexane then 50% acetone in hexane to provide the desired product.

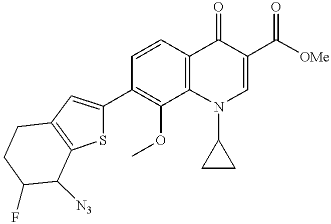

### EXAMPLE 237B

The desired product was prepared by substituting Example 237A for Example 37A in Example 37B and was purified by silica gel chromatography eluting with hexane then 50% acetone in hexane to provide the desired product.

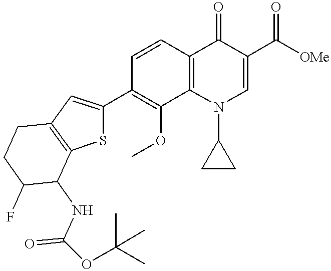

### EXAMPLE 237C

The desired product was prepared by substituting Example 237B for Example 201C in Example 201D.

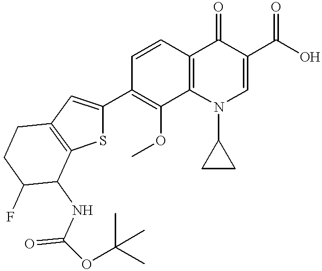

### EXAMPLE 237D

The desired product was prepared by substituting Example 237C for Example 2A in Example 2B.

### EXAMPLE 237E

### 7-(7-amino-6-fluoro-4,5,6,7-tetrahydro-1-benzothien-2-yl)-1-cyclopropyl-8-methoxy-4-oxo-1,4-dihydro-3-quinolinecarboxlic acid hydrochloride

The desired product was prepared by substituting Example 237D for Example 40B in Example 40C and was obtained as a 4:1 (trans:cis)mixture of diastereomers. MS (DCI/NH3) m/z 429 (M+1)⁺; ¹H NMR (500 MHz, DMSO-d₆) δ 8.79 (s, 1H), 8.10 (d, 1H), 7.91 (d, 1H), 7.57 (s, 1H), 5.43-5.18 (m, 1H), 5.86 (dd, 0.2H), 5.71 (dd, 0.8H), 5.25 (m, 1H), 3.67 (s, 3H), 2.82-2.72 (m, 2H), 2.57-1.98 (m, 2H), 1.19-0.95 (m, 4H).

### EXAMPLE 239

### 1-cyclopropyl-8-methoxy-4-oxo-7-(7-oxo-4,5,6,7-tetrahydro-1,3-benzothiazol-2-yl)-1,4-dihydro-3-quinolinecarboxylic acid

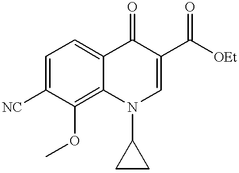

### EXAMPLE 239A

A mixture of Ethyl 7-bromo-1-cyclopropyl-4-oxo-1,4-dihydro-3-quinoline-3-carboxylate (5.0 g, 13.66 mmol), cuprous cyanide (1.6 g, 17.8 mmol) and dimethylformamide was heated to 140° C. for 8 hours. The hot reaction-mixture was poured into a mixture of ice-water (100 mL), and con. NH₄OH ( 15 mL) and the resulting off-white solid in a dark blue colored solution is filtered. The solid was washed with water and extracted with methylene chloride. The organic layer was washed with brine, dried (Na₂SO₄), filtered, and evaporated in vacuo and the residue triturated with ethyl acetate to provide 2.03 g ( 48 %) of the desired product as white solid. M.p. 200-201° C. MS (APCI+) m/z 313 (M+H)⁺; ¹H NMR (300 MHz, DMSO-d₆) δ 8.57 (s, 1 H), 8.06 (d, 1 H), 7.79 (d, 1 H), 4.24 (q, 2 H), 4.06 (m, 1 H), 4.06 (s, 3 H), 1.28 (t, 3 H), 1.13 (m, 2 H), 1.04 (m, 2 H).

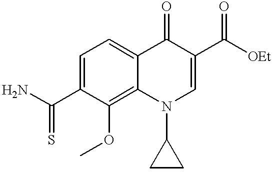

### EXAMPLE 239B

Hydrogen sulfide was passed through a suspension of Example 239A (1.5 g, 4.8 mmol), triethyl amine (2 mL, 15 mmol) and pyridine ( 30 mL) for 0.5 hour. To this dark suspension methylene chloride ( 10 mL) is added and the resulting dark green solution is heated to 50° C. for 12 hours in a sealed tube. After removing the solvents in vacuo, the residue is washed with water, ether and ethanol to afford the desired product as a off-white solid. (0.61 g, 36%). M.p. 224-225° C. MS (APCI+) m/z 347 (M+1)⁺; ¹H NMR (300 MHz, DMSO-d₆) 10.27 (s, 1 H), 9.70 (s, 1 H), 8.55 (s, 1 H), 7.94 (d, 1 H), 7.41 (d, 1 H), 4.23 (q, 2 H), 4.05 (m, 1 H), 3.86 (s, 3 H), 1.28 (t, 3 H), 1.11 (m, 2 H), 1.00 (m, 2 H).

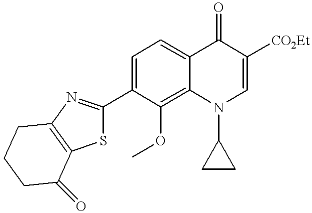

### EXAMPLE 239C

A mixture of Example 239B (0.347 g, 1 mmol) and 2-bromo-3-hydroxycyclohex-2-en-1-one (0.286 g, 1.5 mmol), (prepared by the method described by Shepard and White JCS Perkin 1987, 1, 21 53-21 55,) in DMF (2 mL) was heated to 50° C. for 3 hours in a sealed tube. The resulting solution was diluted with water, the aqueous layer extracted with methylene chloride, the combined organic phases washed with brine, dried (Na₂SO₄), concentrated, and purified by silica gel chromatography eluting with 2 % methanol in methylene chloride to provide the desired product (0.106 g, 24 % yield) as a off-white solid. MS ((APCI+)) m/z 439 (M+H)⁺; ¹H NMR (300 MHz, DMSO-d₆) δ 8.60 (s, 1 H), 8.33 (d, 1 H), 8.11 (d, 1 H), 4.25 (q, 2 H), 4.14 (m, 1 H), 3.84 (s, 3 H), 3.10 (t, 2 H), 2.64 (t, 2 H), 2.20 (m, 2 H), 1.29 (t, 3 H), 1.12 (m, 2 H), 0.94 (m, 2 H).

### EXAMPLE 239D

### 1-cyclopropyl-8-methoxy-4-oxo-7-(7-oxo-4,5,6,7-tetrahydro-1,3-benzothiazol-2-yl)-1,4-dihydro-3-quinolinecarboxylic acid

A solution of Example 239C (0.043 g, 0.1 Immol) was suspended in THF (0.5 mL) and 1M HCl (0.5 mL) and was heated to 100° C. for 10 hours in a sealed tube. Solvent was concentrated in vacuo, and the resulting precipitate that formed filtered and washed with ethyl acetate to give the desired product (0.019 g, 46 %) as a tan solid. MS (APCI+) m/z 411 (M+H)⁺; 1H NMR (300 MHz, DMSO-d₆) δ 14.69 (s, 1 H), 8.86 (s, 1 H), 8.50 (d, 1 H), 8.26 (d, 1 H), 4.32 (m, 1 H), 3.87 (s, 3 H), 3.12 (t, 2 H), 2.65 (t, 2 H), 2.20 (m, 2 H), 1.18 (m, 2 H), 1.04 (m, 2 H).

### EXAMPLE 240

### 1-cyclopropyl-7-(6-fluoro-7-hydroxy-4,5,6,7-tetrahydro-1-benzothien-2-yl)-8-methoxy-4-oxo-1,4-dihydro-3-quinolinecarboxylic acid

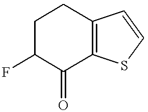

### EXAMPLE 240A

A solution of 7-keto-4,5,6,7-tetrahydrothienanapthene (2.812g, 18.5 mmol, prepared by the method of Caubere, et al. Eur. J. Med. Chem. 1998, 867-77) and N-fluorosulfonimide (10.21 g, 32.4 mmol) in THF (35 mL) was cooled to −78° C., lithum (bistrimethylsilyl)amide (32.4 mL of a 1.0 M solution in THF, 32.4 mmol) was added dropwise, and the mixture was allowed to warm to room temperature overnight. The mixture was partitioned between ethyl acetate and water. The organic phase was washed with brine, dried (Na₂SO₄), and concentrated. The resulting residue was purified by silica gel chromatography eluting with a gradient of hexane to 20% ethyl acetate in hexane to provide the desired product (2.218 g, 70.0%) as a white solid.

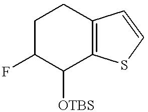

### EXAMPLE 240B

The desired product was prepared by substituting Example 240A for Example 218A in Example 218B to give a 3:1 (cis:trans) mixture of diastereomers.

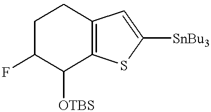

### EXAMPLE 240C

The desired product was prepared by substituting Example 240B for Example 218B in Example 218C.

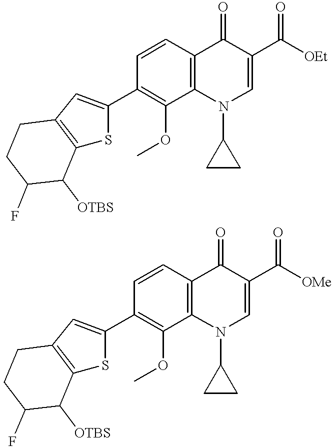

### EXAMPLE 240D EXAMPLE 240E

The desired products were prepared by substituting Example 240C and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E and the crude product substituted for Example 38C in Example 39A. The resulting mixture of the methyl and ethyl esters was separated by silica gel chromatography eluting with a gradient of hexane to 50% acetone in hexane.

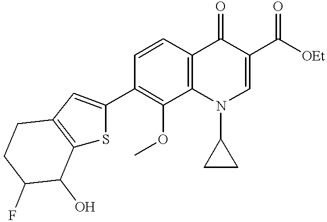

### EXAMPLE 240F

The desired product was prepared by substituting Example 240D for Example 35D in Example 35E and was purified by silica gel chromatography eluting with hexane then 50% acetone in hexane.

### EXAMPLE 240G

### 1-cyclopropyl-7-(6-fluoro-7-hydroxy-4,5,6,7-tetrahydro-1-benzothien-2-yl)-8-methoxy-4-oxo-1,4-dihydro-3-quinolinecarboxylic acid

The desired product was prepared by substituting Example 240F for Example 2A in Example 2B MS (DCI/NH3) m/z 430(M+1)⁺; ¹H NMR (300 MHz, CDCl₃) δ 14.8 (br s, 1H), 8.79 (s, 1H), 8.12, (d, 1H), 7.98 (d, 1H), 7.58 (s, 0.75H), 7.56 (s, 0.25H), 5.05-4.70 (m, 2H), 4.25 (m, 1H), 3.64 (s, 3H), 2.8-2.64 (m, 2H), 2.31-1.90 (m, 2H), 1.20-1.00 (m, 4H).

### EXAMPLE 241

### 1-cyclopropyl-7-(7-hydroxy-6,6-dimethyl-4,5,6,7-tetrahydro-1-benzothien-2-yl)-8-methoxy-4-oxo-1,4-dihydro-3-quinolinecarboxylic acid

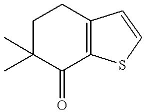

### EXAMPLE 241A

A solution of Example 235A (2.0 g, 12.0 mmol) in anhydrous THF (50 mL)at −78° C. was treated dropwise with lithium bis(trimethylsilyl)amide (13.2 mL of a 1M solution in hexanes, 13.2 mmol), stirred for 30 minutes, treated with methyl iodide (3.4 g, 24.0 mmol), warmed to ambient temperature and partitioned between saturated ammonium chloride and EtOAc. The organic phase was washed with brine, dried (MgSO₄) and concentrated to give the desired compound (2.1 g, 97%) as a yellow oil.

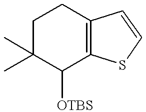

### EXAMPLE 241B

The desired product was prepared by substituting Example 241 A for Example 218A in Example 218B.

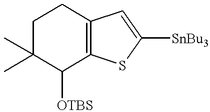

### EXAMPLE 241C

The desired product was prepared by substituting Example 241B for Example 218B in Example 218C.

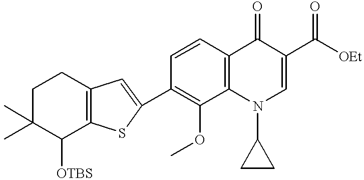

### EXAMPLE 241D

The desired product was prepared by substituting Example 241C, ethyl 1-cyclopropyl-7-bromo-8-methoxy-4-oxo-1,4-dihydroquinoline-3-carboxylate and a reaction time of 4 hours for Example 1D, ethyl 1-cyclopropyl-7-bromo-4-oxo-1,4-dihydroquinoline-3-carboxylate and a reaction time of 24 hours, respectively in Example 1E and was purified by chromatography on silica gel eluting with 2:1 hexane:acetone to provide an off-white solid.

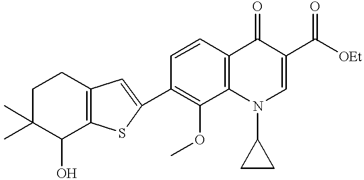

### EXAMPLE 241E

The desired product was prepared by substituting Example 241D for Example 35D in Example 35E and was purified by chromatography on silica eluting with 95:5 dichloromethane:methanol.

### EXAMPLE 241F

### 1-cyclopropyl-7-(7-hydroxy-6,6-dimethyl-4,5,6,7-tetrahydro-1-benzothien-2-yl)-8-methoxy-4-oxo-1,4-dihydro-3-quinolinecarboxylic acid

The desired product was prepared by substituting Example 241E for Example 2A in Example 2B and the crude material triturated with 4:1 Et₂O:hexane (25 mL)and the solid collected by filtration. MS (APCI) m/z 440 (M+H)⁺; 1H NMR (300 MHz, DMSO-d₆) δ 14.94 (s, 1H), 8.79 (s, 11H), 8.10 (d, 1H), 7.99 (d, 1H), 7.55 (s, 1H), 5.49 (d, 1H), 4.36 (d, 1H), 4.26 (m, 1H), 3.69 (s, 3H), 2.60 (m, 2H), 1.74-1.54 (m, 2H), 1.15 (m, 2H), 1.04 (m, 2H), 1.01 (s, 3H), 0.88 (s, 3H).

### EXAMPLE 242

### 1-caclopropyl-7-((6S,7S)-7-hydroxy-6-methyl-4,5,6,7-tetrahydro-1-benzothien-2-yl)-8-methoxy-4-oxo-1,4-dihydro-3-quinolinecarboxylic acid

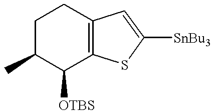

### EXAMPLE 242B

The desired product was prepared by substituting Example 242A in Example 235B for Example 218B in Example 218C.

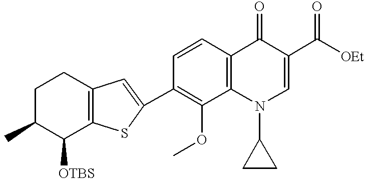

### EXAMPLE 242C

The desired product was prepared by substituting Example 242B, ethyl 1-cyclopropyl-7-bromo-8-methoxy-4-oxo-1,4-dihydroquinoline-3-carboxylate and a reaction time of 2 hours for Example 1D, ethyl 1-cyclopropyl-7-bromo-4-oxo-1,4-dihydroquinoline-3-carboxylate and a reaction time of 24 hours, respectively in Example 1E and purified by chromatography on silica eluting with 2:1 hexane/acetone.

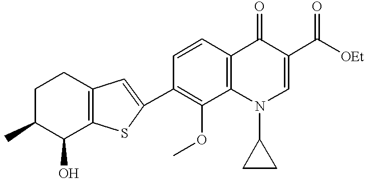

### EXAMPLE 242D

The desired product was prepared by substituting Example 242C for Example 35D in Example 35E and was used without further purification.

### EXAMPLE 242E

### 1-cyclopropyl-7-((6S,7S)-7-hydroxy-6-methyl-4,5,6,7-tetrahydro-1-benzothien-2-yl)-8-methoxy-4-oxo-1,4-dihydro-3-quinolinecarboxylic acid

The desired product was prepared by substituting Example 242D for Example 2A in Example 2B and the crude material purified by triturating with 4:1 Et₂O:hexanes (25 mL) and collecting the solid product by filtration.

MS (APCI) m/z 426 (M+H)⁺; 1H NMR (300 MHz, DMSO- d₆) δ 8.79 (s, 1H), 8.11 (d, 1H), 8.00 (d, 1H), 7.55 (s, 1H), 5.21 (d, 1H), 4.60 (dd, 1H), 4.26 (m, 1H), 3.68 (s, 3H), 2.74-2.55 (m, 2H), 1.95 (m, 1H), 1.76-1.62 (m, 2H), 1.14 (m, 2H), 1.03 (m, 2H), 1.01 (d, 3H).

### EXAMPLE 243

### 1-cyclopropyl-8-methoxy-4-oxo-7-(4,5,6,7-tetrahydro-1-benzothien-2-yl)-1,4-dihydro-3-quinolinecarboxylic acid

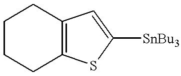

### EXAMPLE 243A

The desired product was prepared by substituting 4,5,6,7-tetrahydrothienanapthene (prepared by the method of Caubere, et al. Eur. J. Med. Chem. 1998, 867-77) for Example 218B in Example 218C.

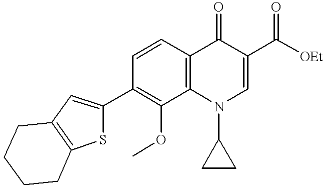

### EXAMPLE 243B

The desired product was prepared by substituting Example 243A and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3 -quinolinecarboxylate, rspectively in Example 1E.

### EXAMPLE 243C

### 1-cyclopropyl-8-methoxy-4-oxo-7-(4,5,6,7-tetrahydro-1-benzothien-2-yl)-1,4-dihydro-3-quinolinecarboxylic acid

The desired product was prepared by substituting Example 243A for Example 2A in Example 2B. MS (DCI/NH₃) m/z 396(M+1)⁺; ¹H NMR (300 MHz, DMSO-d₆) δ 8.78 (s, 1H), 8.10 (d, 1H), 7.98 (d, 1H), 7.56 (s, 1H), 4.27-4.22 (m, 1H), 3.67 (s, 3H), 2.79 (t, 2H), 2.64 (t, 2H), 1.80-1.74 (m, 4H), 1.17-1.03 (m, 4H).

### EXAMPLE 244

### 7-(7-amino-6,6-difluoro-4,5,6,7-tetrahydro-1-benzothien-2-yl)-1-cyclopropyl-8-methoxy-4-oxo-1,4-dihydro-3-quinolinecarboxylic acid hydrochloride

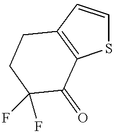

### EXAMPLE 244A

The desired product was prepared by substituting 7-keto-4,5,6,7-tetrahydrothienanapthene (prepared by the method of Caubere, et al. Eur. J. Med. Chem. 1998, 867-77) for 4-keto-4,5,6,7-tetrahydrothienanapthene in Example 358696A.

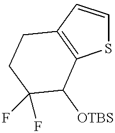

### EXAMPLE 244B

The desired product was prepared by substituting Example 244A for Example 218A and room temperature for heating to 50° C. in Example 218B.

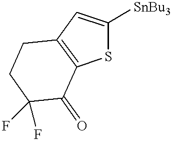

### EXAMPLE 244C

The desired product was prepared by substituting Example 244B for Example 1C in Example 1D.

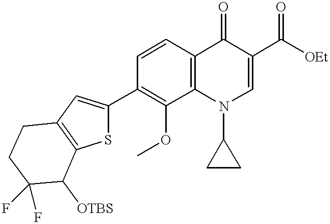

### EXAMPLE 244D

The desired product was prepared by substituting Example 244C, ethyl 7-bromo-1-cyclopropyl-8-methoxy-4-oxo-1,4-dihydro-3-quinolinecarboxylate, and a reaction time of 10 h for Example 1D, ethyl 7-bromo-1-cyclopropyl-4-oxo- 1,4-dihydro-3-quinoline-3-carboxylate and a reaction time of 24 h respectively in Example 1E.

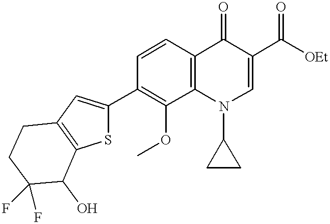

### EXAMPLE 244E

The desired product was prepared by substituting Example 244B for Example 35D in Example 35E and the resulting alcohol was carried on without further purification.

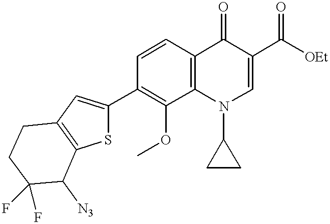

### EXAMPLE 244F

The desired product was prepared by substituting Example 244E for Example 37A in Example 37B and the resulting azide was purified by chromatography on silica gel eluting with 10% acetone in hexane then 20% acetone in hexane.

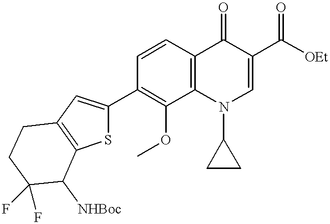

### EXAMPLE 244G

The desired product was prepared by substituting Example 244F for Example 201C in Example 201D.

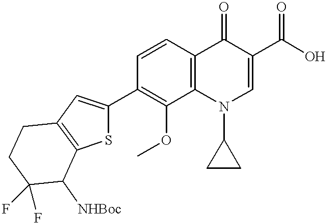

### EXAMPLE 244H

The desired product was prepared by substituting Example 244G for Example 2A in Example 2B.

### EXAMPLE 244I

### 7-(7-amino-6,6-difluoro-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 244H for Example 40B in Example 40C. MS (ESI) m/z 447 (M+H)⁺; ¹H NMR (300 MHz, DMSO-d₆) δ 9.20 (br s, 2H), 8.82 (s, 1H), 8.17 (d, 1H), 8.05 (d, 1H), 7.74 (s, 1H), 5.22 (m, 1H), 4.28 (m, 1H), 4.00 (m, 2H), 3.73 (s, 3H), 2.94 (t, 2H), 1.10 (m, 4H).

### EXAMPLE 245

### 7-(7-amino-6,6-dimethyl-4,5,6,7-tetrahydro-1-benzothien-2-yl)-1-cyclopropyl-8-methoxy-4-oxo-1,4-dihydro-3-quinolinecarboxylic acid hydrochloride

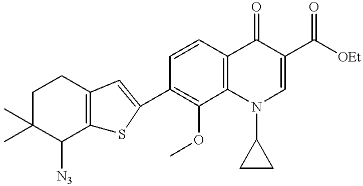

### EXAMPLE 245A

The desired product was prepared by substituting Example 241E for Example 37A in Example 37B and purified by chromatography on silica eluting with 2:1 hexanes:acetone.

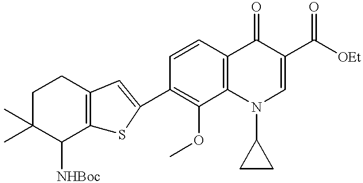

### EXAMPLE 245B

The desired product was prepared by substituting Example 245A for Example 201C in Example 201D.

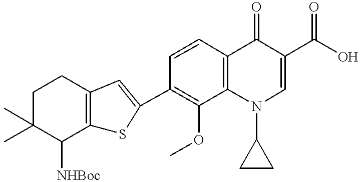

### EXAMPLE 245C

The desired product was prepared by substituting Example 245B for Example 2A in Example 2B.

### EXAMPLE 245D

### 7-(7-amino-6,6-dimethyl-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 245C for Example 40B in Example 40C. MS (APCI) m/z 439 (M+H)⁺; 1H NMR (300 MHz, DMSO-d₆) δ 8.81 (s, 1H), 8.42 (bs, 3H), 8.15 (d, 1H), 8.04 (d, 1H), 7.68 (s, 1H), 4.27 (m, 2H), 3.73 (s, 311), 2.68 (m, 211), 1.87 (m, 1H1), 1.63 (m, 111), 1.20-0.95 (m, 4H), 1.08 (s, 6H).

### EXAMPLE 246

### 1-cyclopropyl-6-fluoro-8-methoxy-7-(7-(methylamino)-4,5,6,7-tetrhydro-1-benzothien-2-yl)-4-oxo-1,4-dihydro-3-quinolinecarboxiylic acid hydrochloride

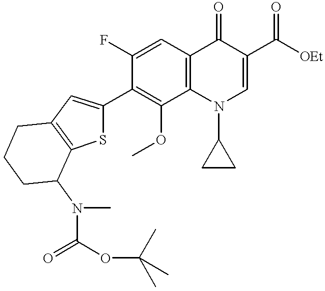

### EXAMPLE 246A

The desired product was prepared by substituting Example 216B and ethyl 7-bromo-1-cyclopropyl-6-fluoro-8-methoxy-4-oxo-1,4-dihydro-3-quinolinecarboxylate for Example 1D and ethyl 7-bromo-1-cyclopropyl-6-fluoro-4-oxo-1,4-dihydro-3-quinolinecarboxylate, respectively in Example 1E.

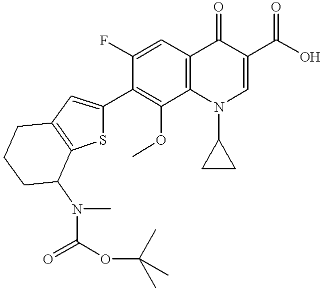

### EXAMPLE 246B

The desired product was prepared by substituting Example 246A for Example 2A in Example 2B.

### EXAMPLE 246C

### 1-cyclopropyl-6-fluoro-8-methoxy-7-(7-(methylamino)-4,5,6,7-tetrahydro-1-benzothien-2-yl)-4-oxo-1,4-dihydro-3- uinolinecarbolcylic acid hydrochloride

The desired product was prepared by substituting Example 246B for Example 40B in Example 40C. MS (DCI/NH₃) m/z 443 (M+1)⁺; ¹H NMR (300 MHz, MeOH-d₄) δ 8.95 (s, 1H), 7.98 (d, 1H), 7.48 (d, 1H1), 4.64 (t, 1H1), 4.26 (m, 1H), 3.68 (s, 3H), 2.95- 2.74 (mn, 5H), 2.30-2.24 (mn, 2H), 2.06-1.99 (mn, 2H), 1.52-1.10 (m, 4H).

### EXAMPLE 247

### 1-cyclopropyl-8-methoxy-4-oxo-7-(5-oxo-5,6-dihydro-4H-cyclopenta[b]thien-2yl-)1,4-dihydro-3-quinolinecarboxylic acid

### EXAMPLE 247A

A solution of Example 45G (0.500 g, 1.18 mmol) in dichloromethane (12 mL) was treated with the Dess-Martin periodinane (798 mg, 1.88 mmol) followed by stirring at ambient temperature for 3 h. The solution was diluted with dichloromethane and added to a solution of sodium thiosulfate (3.50 g, 14.10 mmol) in saturated sodium bicarbonate solution (60 mL). The mixture was stirred for 30 min and the layers separated. The aqueous phase was extracted with dichloromethane, and the combined organic layers were dried over Na₂SO₄. Concentration in vacuo afforded a beige solid which was purified by chromatography on silica gel eluting with chloroform and then with methanol in chloroform to afford the desired material (0.399 g, 80%) as a white solid.

### EXAMPLE 247B

### 1-cyclopropyl-8-methoxy-4-oxo-7-(5-oxo-5,6-dihydro-4H-cyclopenta[b]thien-2-yl)-1,4-dihydro-3-quinolinecarboxylic acid

A solution of Example 247A (0.129 g, 0.30 mmol) in 3 mL THF and 1 mL ethanol was treated with 0.91 mL 1.0 NHCl solution followed by warming at reflux for 5 h. The solution was cooled and concentrated in vacuo with ethanol to remove water. The purple solid was triturated with 2:1 ethanol-water, collected by filtration, and washed with ether. The solid was dried in a vacuum oven (50° C./10 mm Hg) for 18 h to afford the desired product (0.086 g, 72%) as a purple solid. MS (ESI) m/z 396 (M+H)⁺; ¹H NMR (DMSO-d₆, 300 MHz) δ 8.80 (s, 1 H), 8.13 (d, 1 H), 8.05 (d, 1 H), 7.81 (s, 1 H), 4.26 (m, 1 H), 3.70 (s, 3 H), 3.65 (br s, 2 H), 3.48 (br s, 2 H), 1.16 (m, 2 H), 1.06 (m, 2 H).

### EXAMPLE 248

### 1-cyclopropyl-8-methoxy-7-((5E/Z)-5-(methoxyimino)-5,6-dihydro-4H-cyclopenta[b]thien-2-yl)-4-oxo-1,4-dihydro-3-quinolinecarboxylic acid

The desired compound was prepared by substituting Example 247B for Example 50 in Example 325004 to give a purple solid. MS (ESI) m/z 425 (M+H)⁺; ¹H NMR (DMSO-d₆, 300 MHz) δ 14.82 (br s, 1 H), 8.79 (s, 1 H), 8.11 (d, 1 H), 8.00 (d, 1 H), 7.73, 7.71 (s, 1 H), 4.25 (br m, 1 H), 3.85 (s, 3 H), 3.80 (d, 2 H), 3.69 (s, 3 H), 3.61 (d, 2 H), 1.16 (m, 2 H), 1.04 (m, 2 H).

### EXAMPLE 249

### 7-(5-amino-5,6-dihydro-4H-cyclopenta[b]thien-2-yl)-1-cyclopropyl-8-methoxy-4-oxo-1,4-dihydro-3-quinolinecarboxylic acid hydrochloride

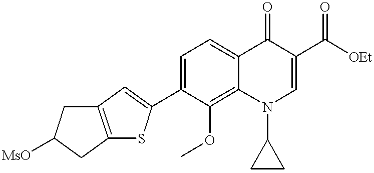

### EXAMPLE 249A

A solution of Example 45G (0.504 g, 1.30 mmol), and triethylamine (0.9 mL, 6.50 mmol) in dichloromethane (6.5 mL) at 0° C. was treated dropwise with methanesulfonyl chloride (0.25 mL, 2.20 mmol), followed by stirring for 30 min. The solution was warmed to ambient temperature, diluted with dichloromethane (30 mL) and extracted with water (2×), 1.0 M citric acid, and saturated sodium bicarbonate. Drying (Na₂SO₄) and concentration in vacuo afforded the desired product (0.666 g, 100%) as an off-white foam.

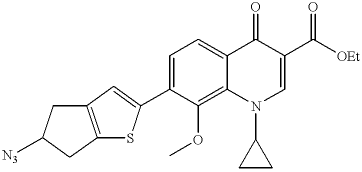

### EXAMPLE 249B

A solution of Example 249A (0.655g, 1.30mmol) and sodium azide (0.846 g, 13.0 mmol) in DMF (16 mL) was warmed at 60° C. for 45 min, followed by cooling to ambient temperature for 18 h. The solution was diluted with ethyl acetate (100 mL) and washed with water (5×20 mL) and then with saturated NaCl solution. Drying (Na₂SO₄) and concentration in vacuo afforded the desired product (0.530 g, 90%) as a buff-colored solid.

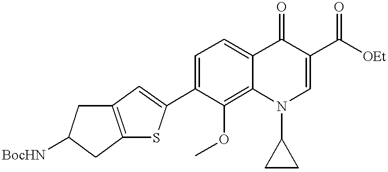

### EXAMPLE 249C

The desired compound was prepared by substituting Example 324124B for Example 201C in Example 201D.

### EXAMPLE 249D

### 7-(5-amino-5,6-dihydro-4H-cyclopenta[b]thien-2-yl)-1-cyclopropyl-8-methoxy-4-oxo-1,4-dihydro-3-quinolinecarboxylic acid hydrochloride

A solution of Example 249C (0.067 g, 0.13 mmol) in THF (5 mL) and 1.0 N HCl solution (4 mL) was warmed at reflux for 5 h. The solution was cooled to ambient temperature and concentrated in vacuo. The residue was suspended in ethanol and concentrated in vacuo twice. The material was suspended in dichloromethane and treated with 5 mL of 4.0 N HCl in dioxane, followed by stirring at ambient temperature for 1 h. The solid was collected by filtration and washed with ether. These procedures afforded the desired product (0.043 g, 86%) as an orange solid. MS (ESI) m/z 397 (M+H)⁺; ¹H NMR (DMSO-d₆, 300 MHz) δ 8.79 (s, 1 H), 8.49 (br s, 1 H), 8.11 (d, 1 H), 7.98 (d, 1 H), 7.69 (s, 1 H), 4.34 (br m, 1 H), 4.27 (m, 1 H), 3.69 (s, 3 H), 3.40 (dd, 1 H), 3.21 (dd, 1 H), 3.06 (dd, 1 H), 2.87 (dd, 1 H), 1.16 (m, 2 H), 1.04 (m, 2 H).

### EXAMPLE 250

### 1-cyclopropyl-7-(5-((ethoxycarbonyl)amino)-5,6-dihydro-4H-cyclopenta[b]thien-2-yl)-8-methoxy-4-oxo-1,4-dihydro-3-quinolinecarboxmlic acid

The desired product was prepared by substituting ethyl chloroformate for methyl chloroformate in Example 251. MS (ESI) m/z 469 (M+H)⁺; ¹H NMR (CDCl₃, 300 MHz) δ 14.73 (s, 1 H), 8.91 (s, 1 H), 8.23 (d, 1H), 7.73 (d, 1 H), 7.41 (s, 1 H), 4.80 (br m, 1 H), 4.12 (om, 3 H), 3.67 (s, 3 H), 3.42 (dd, 1 H), 3.26 (dd, 1 H), 2.87 (dd, 1 H), 2.70 (dd, 1 H), 1.26 (om, 5 H), 1.11 (m, 2 H).

### EXAMPLE 251

### 1-cyclopropyl-8-methoxy-7-(5-((methoxvycarbonyl)amino)-5,6-dihydro-4H-cyclopenta[b]thien-2-yl)-4-oxo-1,4-dihydro-3-quiunolinecarboxylic acid

A solution of Example 249 (0.041 mig, 0. 10 mmol) in 3 mL acetone and 1 mL saturated sodium bicarbonate solution was treated with methyl chloroformate (12 μL, 0.16 mmol) followed by stirring at ambient temperature for 2 h. The mixture was adjusted to pH 2 by addition of 1.0 N HCl solution. The mixture was adsorbed onto a Varian Chem-Elut CE 1010 cartridge, and after equilibration for 5 min, the product was eluted with methanol in dichloromethane. The material was purified by preparative reverse-phase HPLC. These procedures afforded the desired product (21 mig, 45%) as a light yellow solid. MS (ESI) m/z 455 (M+H)⁺; ¹H NMR (CDCl₃, 300 MHz) δ 14.73 (s, I H), 8.91 (s, I H), 8.23 (d, I H), 7.73 (d, I H), 4.89 (br m, 1 H), 4.11 (m, 1H), 3.71 (br s, 3 H), 3.67 (s, 3 H), 3.43 (dd, I H), 3.26 (dd, I H), 2.87 (dd, 1 H), 2.69 (dd, 1 H), 1.28 (ni, 2 H), 1.06 (m, 2 H).

### EXAMPLE 252

### 7-(5-(acetylamino)-5,6-dihydro-4H-cyclopenta[b]thien-2-yl)-1-cyclopropyl-8-methoxy-4-oxo-1,4-dihydro-3-quinolinecarboxylic acid

A solution of Example 249 (0.015 g, 0.04 mmol), 1 mL triethylamine, and 3 mg N, N-dimethylaminopyridine in dichloromethane (2 mL) was treated with acetic anhydride (250 μ). The solution was stirred at ambient temperature for 2 h. The solution was diluted with dichloromethane and filtered through a Varian Chem-Elut CE1010 (which had been pretreated with 2 mL water and 2 mL 1 N HCl solution), eluting with dichloromethane. The filtrate was concentrated in vacuo to afford a yellow solid which was purified by preparative HPLC to give the desired product (0.011 g, 64%) as a yellow solid. MS (ESI) m/z 439 (M+H)⁺; ¹H NMR (CDCl₃, 300 MHz) δ 14.73 (s, 1 H), 8.88 (s, 1 H), 8.20 (d, 1H), 7.71 (s, 1 H), 5.98 (brd, 1H), 5.11 (m, 1 H), 4.10 (m, 1 H), 3.67 (s, 3 H), 3.45 (dd, 1 H), 3.28 (dd, 1 H), 2.85 (dd, 1 H), 2.69 (dd, 1 H), 2.02 (s, 3 H), 1.28 (m, 2 H), 1.07 (m, 2 H).

### EXAMPLE 253

### 1-cycloopropyl-8-methoxy-7-(5-(((4-methylphenyl)sulfonyl)amino)-5,6-dihydro-4H-cyclopenta[b]thien-2-yl)4-oxo-1,4-dihydro-3-quinolinecarboxylic acid

A solution of Example 249 (0.041 g, 0.10 mmol) and triethylamine (36 μL, 0.26 mmol) in 3 mL dichloromethane was treated with p-toluenesulfonyl chloride (0.030 g, 0.16 mmol) followed by stirring at ambient temperature for 2 h. The solution was diluted with dichloromethane and filtered through a Varian Chem-Elut CE 1010 cartridge (which had been pretreated with 2 mL water and 2 mL 1 N HCl solution) eluting with methanol in dichloromethane. The filtrate was concentrated in vacuo to afford a yellow solid, which was purified by preparative HPLC to give the desired compound (0.0081 g, 14%) as an off-white solid. MS (ESI) m/z 551 (M+H)⁺; ¹H NMR (CDCl₃, 300 MHz) δ 14.72 (s, 1 H), 8.89 (s, 1 H), 8.20 (d, 1 H), 7.82 (d, 2 H), 7.68 (d, 1 H), 7.34 (d, 2 H), 5.04 (br d, 1 H), 4.51 (m, 1 H), 4.08 (m, 1 H), 3.64 (s, 3 H), 3.25 (dd, 1 H), 3.09 (dd, 1 H), 2.83 (dd, 1 H), 2.65 (dd, 1 H), 2.47 (s, 3 H), 1.27 (m, 2 H), 1.03 (m, 2H).

### EXAMPLE 254

### 1-cyclopropyl-8-methoxy-7-(5-((methylsulfonyl)amino)-5,6-dihydro-4H-cyclopenta[b]thien-2-yl)-4-oxo-1,4-dihydro-3-quinolinecarboxylic acid

A solution of Example 249 (0.039 g, 0.10 mmol) and triethylamine (35 μL, 0.25 mmol) in 3 mL dichloromethane was treated with methanesulfonyl chloride (12 μL, 0.15 mmol) followed by stirring at ambient temperature for 2 h. The solution was diluted with dichloromethane and filtered through a Varian Chem-Elut CE1010 cartridge (which had been pretreated with 2 mL water and 2 mL 1 N HCl solution) eluting with dichloromethane. The filtrate was concentrated in vacuo to afford a yellow solid which was purified by preparative HPLC to give the desired product (0.013g, 27%) as a beige solid. MS (ESI) m/z 475 (M+H)⁺; ¹H NMR (CDCl₃, 300 MHz) δ 14.72 (s, 1 H), 8.90 (s, 1 H), 8.23 (d, 1 H), 7.72 (d, 1 H), 7.41 (s, 1 H), 4.83 (br d, 1 H), 4.68 (m, 1 H), 4.09 (m, 1 H), 3.67 (s, 3 H), 3.48 (dd, 1 H), 3.33 (dd, 1 H), 3.06 (s, 3 H), 3.02 (dd, 1 H), 2.82 (dd, 1 H), 1.28 (m, 2 H), 1.06 (m, 2 H).

### EXAMPLE 255

### 7-((5E/Z)-5-((benzyloxyimino)-5,6-dihydro-4H-cyclopenta[b]thien-2-yl)-1-cyclopropyl-8-methoxy-4-oxo-1,4-dihydro-3-quinolinecarboxvlic acid

The desired compound was prepared by substituting Example 247B and O-benzylhydroxylamine hydrochloride for Example 50 and methoxylamine hydrochloride, respectively in Example 64. MS (ESI) m/z 501 (M+H)⁺; ¹H NMR (DMSO-d₆, 300 MHz) δ 14.91 (s, 1H), 8.79 (s, 1H), 8.11 (d, 1 H), 7.73, 7.71 (s, 1H), 7.35 (om, 5H), 5.13 (s, 2H), 4.25 (m, 1H), 3.84 (d, 2H), 3.69 (s, 3H), 3.64 (d. 2H), 1.15 (m, 2H), 1.05 (m, 2H).

### EXAMPLE 256

### 1-cyclopropyl-7-((5E/Z)-5 -(hydroxyimino)-5,6-dihydro-4H-caclopenta[b]thien-2-yl)-8-methoxy-4-oxo-1,4-dihydro-3-quinolinecarboxylic acid

The desired compound was prepared by substituting Example 247B and hydroxylamine hydrochloride for Example 50 and methoxylamine hydrochloride, respectively in Example 325004. MS (ESI) m/z 411 (M+H)⁺; ¹H NMR (DMSO-d₆, 300 MHz) δ 14.91 (s, 1H), 10.82, 10.81 (s, 1H), 8.79 (s, 1 H), 8.11 (d, 1H), 8.01 (d, 1H), 7.74,7.73 (s, 1H), 4.26 (m, 1H), 3.77 (d, 2H), 3.69 (s, 3H), 3.58 (d, 2H), 1.16 (m, 2H), 1.05 (m, 2H).

### EXAMPLE 257

### 7-(4-amino-4-methyl-4,5 6,7-tetrahydro-1-benzothien-2-yl)-1-cyclopropyl-8-methoxy-4-oxo-1,4-dihydro-3-quinolinecarboxylic acid

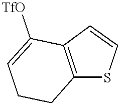

### EXAMPLE 257A

A solution of 4-keto-4,5,6,7-tetrahydrothianapthene (8.0g, 52.58 mmol) and 2,6-di-tert-butyl-4-methylpyridine (11.87g, 57.83 mmol) in dichloromethane (210 mL) at 0° C. was treated dropwise with triflic anhydride (10.7 mL, 63.62 mmol). The mixture was stirred at 0° C. for 10 min, followed by warming to ambient temperature for 30 min. The mixture was concentrated in vacuo and the residue was slurried in 1:1 ethyl acetate-hexane. The mixture was filtered through a pad of celite and the filtrate was concentrated in vacuo. The oil obtained was purified by silica gel chromatography eluting with hexane and then with ethyl acetate in hexane to give the desired product (10.9 g, 73%) as a dark red oil.

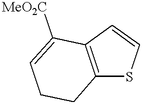

### EXAMPLE 257B

A solution of Example 257a (10.86 g, 38.21 mmol), triethyl amine (10.9 mL), methanol (65.6 mL), palladium acetate (514 mg, 2.29 mmol), and triphenylphosphine (1.20g, 4.58 mmol) in DMF (190 mL) was warmed at 50° C. under an atmosphere of carbon monoxide (4 atmospheres pressure) for 2 h. The solution was cooled to ambient temperature and vented to the atmosphere. The DMF was removed by short path vacuum distillation (oil bath temperature 55° C., 0.4 mm Hg). The residue was dissolved in 250 mL ethyl acetate and extracted with water (2×100 mL) and saturated sodium bicarbonate solution (100 mL). Drying (Na₂SO₄) and concentration in vacuo afforded a brown oil which was purified by silica gel chromatography eluting with hexane and then with ethyl acetate in hexane to give the desired compound (5.8 g, 67%) as a colorless oil.

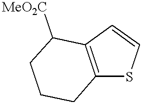

### EXAMPLE 257C

A solution of Example 257B (3.0 g, 15.44 mmol) in anhydrous methanol (37 mL) was treated with magnesium turnings (863 mg, 35.53 mmol) followed by stirring at ambient temperature for 18 h. The solution was treated with saturated ammonium chloride solution (30 mL) followed by dilution with water and ethyl acetate. The aqueous layer was extracted with ethyl acetate and the combined organic layers were dried (Na₂SO₄). Concentration in vacuo afforded an amber oil which was purifed by silica gel chromatography eluting with hexane and then with ethyl acetate in hexane to give the desired product (2.36 g, 78%) as a colorless oil.

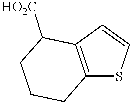

### EXAMPLE 257D

A solution of Example 257C (2.36 g, 12.02 mmol) in 1.0 NNaOH solution (47 mL) was warmed at reflux for 3 h. The solution was cooled to 0+ C. and adjusted to pH 2 by addition of 6 N HCl solution. The mixture was extracted with ethyl acetate (3×75 nlL) and the combined organic layers were dried (Na₂SO₄) and concentrated in vacuo to afford the desired compound (2.14 g, 98%) as a light amber solid.

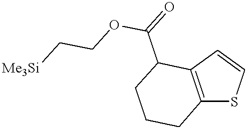

### EXAMPLE 257E

A solution of Example 257D (2.14 g, 11.75 mmol) in dichloromethane (40 mL) was treated with a solution of oxalyl chloride in dichloromethane (6.5 mL, 2.0 M, 12.90 mmol) followed by stirring at ambient temperature for 2.5 h. The solution was concentrated in vacuo to approximately one third original volume and the concentrate was diluted with dichlormethane (30 mL). The solution was cooled to 0° C., and treated sequentially with triethylamine (4.1 mL, 29.36 mmol) and N, N-dimethylaminopyridine (360 mg, 2.94 mmol). The solution was then treated with 2-(trimethylsilyl)ethanol (2.10 mL, 14.68 mmol) followed by stirring at 0° C. for 30 min, and then warming to ambient temperature for 1 h. The solution was diluted with dichloromethane and extracted with water (3x). Drying (Na₂SO₄) and concentration in vacuo afforded an orange oil which was purified by silica gel chromatography eluting with hexane and then with ethyl acetate in hexane. These procedures afforded the desired product (3.02 g, 91%) as a colorless oil.

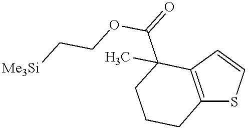

### EXAMPLE 257F

A solution of Example 257E (3.02 g, 10.69 mmol) in THF (43 mL) at −78° C. was treated dropwise over 10 min with a solution of lithium hexamethyldisilylazide (15.0 mL, 14.97 mmol) followed by stirring at −78° C. for 1 h. The solution was treated with a solution of methyl iodide in methyl-tert-butyl ether (10.7 mL, 21.38 mmol) followed by warming to 0° C. for 1 h, and then warming to ambient temperature for 18 h. The solution was treated with saturated ammonium chloride solution (5 mL), followed by dilution with water and ethyl acetate. The organic layer was extracted with water (2×) and saturated sodium chloride solution. Drying (Na₂SO₄) and concentration in vacuo afforded an amber oil which was purified by silica gel chromatography eluting with hexane and then with ethyl acetate in hexane to give the desired product (2.59 g, 83%) as a colorless oil.

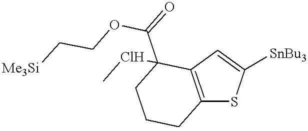

### EXAMPLE 257G

A solution of 2,2,6,6-tetramethylpiperidine (0.24 mL, 1.43 mmol) in THF (6 mL) at −78° C. was treated with a solution of n-butyllithium in hexane (0.53 mL, 2.5 M, 1.32 mmol) followed by warming at −15° C. for 4 min, and cooling again to −78° C. To this solution was added via cannula a cold (−78° C.) solution of Example 257F (0.327 g, 1.10 mmol) in 4 mL THF. The orange solution was stirred at −78° C. for 2 h. The solution was treated with tri-n-butylchlorostannane (0.36 mL, 1.32 mmol) dropwise followed by stirring at −78° C. for 15 min, warming to −40° C. for 30 min, and then to 0° C. and finally ambient temperature. The solution was stirred at ambient temperature for 30 min and was added to cold (0° C.) saturated ammonium chloride solution followed by dilution with ethyl acetate and water. The organic layer was extracted with saturated ammonium chloride solution (2x) and saturated sodium chloride solution. Drying (Na₂SO₄) and concentration in vacuo afforded the desired product (0.729 g, ca. 100%) as a colorless oil.

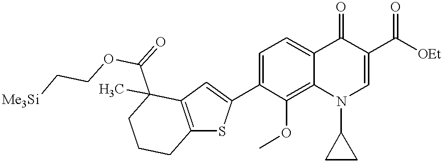

### EXAMPLE 257H

A solution of Example 257G (0.646 mg, 1.10 mmol), ethyl 7-bromo-1-cyclopropyl-8-methoxy-4-oxo-1,4-dihydro-3-quinolinecarboxylate (0.202 g, 0.55 mmol), bis(triphenylphosphine)palladium (II) chloride (0.040 g, 0.06 mmol) in 6 mL toluene was degassed by alternate evacuation and purging with nitrogen. The solution was warmed at 95° C. for 3 h, followed by cooling to ambient temperature. The solution was diluted with ethyl acetate and extracted with saturated ammonium chloride solution. The mixture was filtered through a pad of celite and the layers of the filtrate were separated. The organic phase was extracted with saturated ammonium chloride solution (2x) and with saturated sodium chloride solution. Drying (Na₂SO₄) and concentration in vacuo afforded a brown oil which was dissolved in dichloromethane and treated with an excess of diazoethane (prepared from N-ethyl-N-nitrosourea in dichloromethane by the method of Marshall: Marshall, J. A.; Partridge, J. J. J. Org. Chem. 1968, 33, 4090) followed by allowing the resulting solution to stand at ambient temperature for 18 h. The solution was dried (MgSO₄) and concentrated in vacuo to afford an amber oil. This material was purified by silica gel chromatography, eluting with ethyl acetate in hexane to give the desired product (0.106 g, 33%) as an off-white solid.

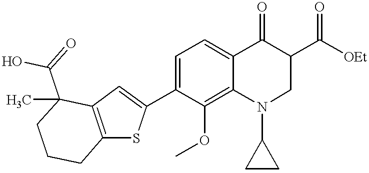

### EXAMPLE 257I

A solution of Example 257h (0.099 g, 0.17 mmol) in THF (2.0 mL) was treated with a solution of tetrabuylammonium fluoride (0.34 mL, 1.0 M, 0.34 mmol) followed by stirring at ambient temperature for 30 min. The solution was diluted with dichloromethane and extracted with water (3×). The solution was dried (Na₂SO₄) and concentrated in vacuo to afford the desired product (0.081 mg, 100%) as an off-white solid.

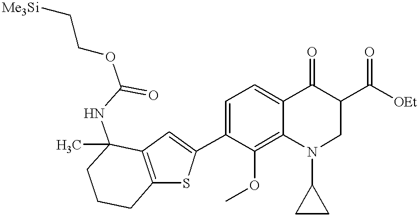

### EXAMPLE 257J

A solution of Example 2571 (0.081 g, 0.17 mmol) and triethylamine (28 μL, 0.20 mmol) in toluene (1.5 mL) was treated with diphenylphosphoroyl azide (37 μL, 0.17 mmol) followed by warming at 80° C. for 3 h. The solution was cooled to ambient temperature and treated with 2-(trimethylsilyl)ethanol (73 μL, 0.51 mmol) followed by warming at 60° C. for 2 h. The solution was cooled to ambient temperature, diluted with dichloromethane and extracted with water (3×). Drying (Na₂SO₄) and concentration in vacuo afforded an oil which was purified by silica gel chromatography eluting with ethyl acetate in hexane to give the desired product (0.053 g, 52%) as an oil.

### EXAMPLE 257K

### 7-(4-amino-4-methyl-4,5,6,7-tetrahydro-1-benzothien-2-yl)-1-cyclopropyl-8-methoxy-4-oxo-1,4-dihydro-3-quinolinecarboxylic acid

A solution of Example 257J (0.047 g, 0.08 mmol) in THF (1.0 mL) was treated with a solution of tetrabutylammonium fluoride (0.16 mL, 1.0 M, 0.16 mmol) followed by warming at 70° C. for 1 h. The solution was cooled to ambient temperature and diluted with chloroform, followed by extraction with water (2×). Drying (Na₂SO₄) and concentration in vacuo afforded an oil. This material was dissolved in THF (1.0 mL) and water (0.5 mL) and treated with lithium hydroxide monohydrate (17 mg, 0.39 mmol) followed by stirring at ambient temperature for 1 h. The mixture was concentrated in vacuo and the residue was dissolved in water (5 mL). The solution was adjusted to pH 7 by addition of 1 N HCl solution, followed by extraction with dichloromethane (5×). The organic phase was dried (Na₂SO₄) and concentrated in vacuo to afford, after trituration with chlorform-hexane, the desired product (0.018 g, 54%) as a buff-colored solid. MS (ESI) m/z 425 (M+H)⁺; ¹H NMR (DMSO-d₆, 300 MHz) δ 8.78 (s, 1 H), 8.11 (d, 1 H), 8.01 (d, 1 H), 7.93 (s, 1 H), 4.24 (m, 1 H), 3.68 (s, 3 H), 2.75 (t, 2 H), 1.93 (m, 1 H), 1.82 (I, 1 H), 1.71 (m, 2 H), 1.33 (s, 3 H), 1.24 (m, 1H), 1.15 (m, 2 H), 1.03 (m, 2 H).

### EXAMPLE 258

### 1-cyclopropyl-7-(5-(dimethylamino)-5,6-dihydro-4H-cyclopenta[b]thien-2-yl)-8-methoxy-4-oxo-1,4-dihydro-3-quinolinecarboxylic acid hydrochloride

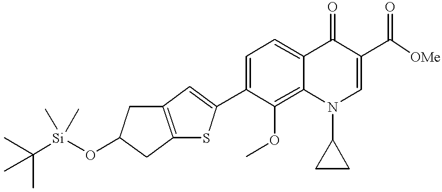

### EXAMPLE 258A

The desired product was prepared by substituting Example 45F for Example 38 C in Example 39A and was used without firther purificat ion.

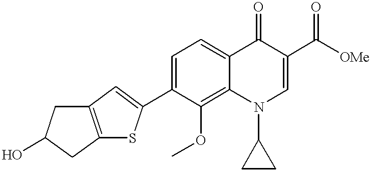

### EXAMPLE 258B

The desired product was prepared by substituting Example 258A for Example 45F in Example 45G.

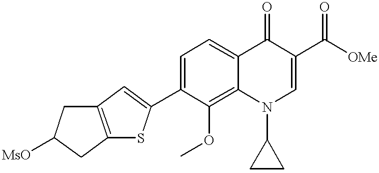

### EXAMPLE 258C

The desired product was prepared by substituting Example 258B for Example 45G in Example 249A.

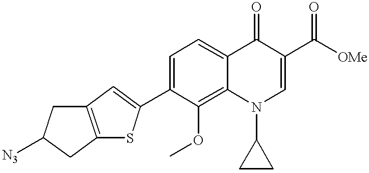

### EXAMPLE 258D

The desired product was prepared by substituting Example 258C for Example 249A in Example 249B.

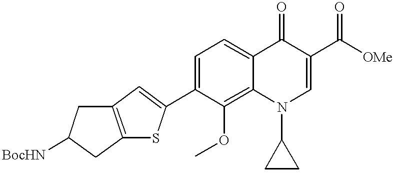

### EXAMPLE 258E

The desired product was prepared by substituting Example 258D and 10:1 dichloromethane:DMF as reaction solvent for Example 201C and dichloromethane as reaction solvent, respectively in Example 35366D.

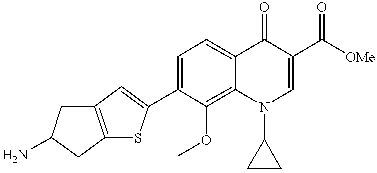

### EXAMPLE 258F

The desired product was prepared by substituting Example 258E for Example 40B in Example 40C.

### EXAMPLE 258G

### 1-cyclopropyl-7-(5-(dimethylamino)-5,6-dihydro-4H-cyclopenta[b]thien-2-yl)-8-methoxy-4-oxo-1,4-dihydro-3-quinolinecarboxylic acid hydrochloride

A solution of Example 258F (0.096 g, 0.22 mmol) in 6.42 mL of 37% formaldehyde solution was treated with 1.6 mL of 88% formic acid, followed by warming at 90° C. for 24 h. The solution was cooled to ambient temperature and concentrated in vacuo, removing the last traces of water by azeotroping with ethanol. The residue was suspended in dichloromethane and filtered through a pad of celite to remove insoluble materials. The filtrate was treated with 4 N HCl in dioxane. The precipitate was collected by filtration and washed with ether to give the desired product (0.050 g, 51%) as a yellow solid. MS (ESI) m/z 425 (M+H)⁺; ¹H NMR (CDCl₃, 300 MHz) δ 8.91 (s, 1 H), 8.32 (s, 1 H), 8.24 (d, 1 H), 7.72 (d, 1 H), 7.40 (s, 1 H), 4.18 (m, 1 H), 4.11 (m, 1H), 3.66 (s,.3H), 3.37 (d, 2 H), 3.19 (d, 2H), 2.71 (s, 6 H), 1.28 (m, 2 H), 1.06 (m, 2 H).

### EXAMPLE 259

### 7-(4-azido-5,5-dimethyl-4,5,6,7-tetrahydro-1-benzothien-2-yl)-1-cyclopropyl-8-methoxy-4-oxo-1,4-dihydro-3-quinolinecarboxylic acid

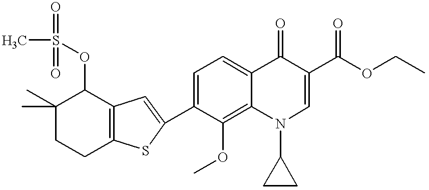

### EXAMPLE 259A

The desired product was prepared by substituting Example 213E for Example 45G in Example 249A.

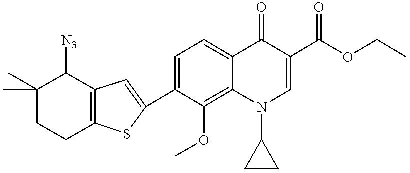

### EXAMPLE 259B

The desired product was prepared by substituting Example 259A and DMSO as reaction solvent for Example 249A and DMF as reaction solvent in Example 249B.

### EXAMPLE 259C

### 7-(4-azido-5,5-dimethyl-4,5,6,7-tetrahydro-1-benzothien-2-yl)-1-cyclopropyl-8-methoxy-4-oxo-1,4-dihydro-3-quinolinecarboxylic acid

The desired product was prepared by substituting Example 259B for Example 2A in Example 2B. mp 89-90° C.; MS (APCI) m/z 465 (M+H)⁺; ¹H NMR (300 MHz, DMSO-d₆) δ 8.90 (s, 1H), 8.24 (d, 1H), 7.75 (d, 1H), 7.53 (s, 1H), 4.13 (m, 2H), 3.68 (s, 3H), 2.90 (m, 2H), 1.90 (m, 2H), 1.53 (br s, 1H), 1.28 (m, 2H), 1.13 (s, 3H), 1.09 (s, 3H), 1.07 (m, 2H).

### EXAMPLE 260

### 7-(7-amino-4,5,6,7-tefrahydro-1-benzothien-3-yl)-1-cyclopropyl-8-methoxy-4-oxo-1,4-dihydro-3-quinolinecarboxylic acid

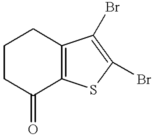

### EXAMPLE 260A

A solution of 7-keto-4,5,6,7-tetrahydrothienanapthene (4.00g, 26.3 mmol, prepared by the method of Caubere, et al. Eur. J. Med. Chem. 1998, 867-77) in 50% aqueous acetic acid was cooled to 0° C. and treated dropwise with a solution of bromine (6 mL, 103 mmol) in 50% aqueous acetic acid. The mixture was allowed to warm to room temperature and stirred overnight. Saturated aqueous sodium acetate (50 mL) was added. The precipitate was filtered, washed with water and dried under vacuum to provide the desired product (8.07 g, 99%) as a white solid.

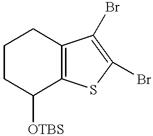

### EXAMPLE 260B

The desired product was prepared by substituting Example 260A for Example 218A in Example 218B.

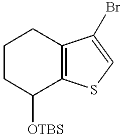

### EXAMPLE 260C

A solution of Example 260B in diethyl ether was cooled to −78° C. and treated dropwise with n-butyl lithium (2.2 mL of a 2.5 M solution in hexanes, 5.5 mmol). The reaction mixture was stirred at −78° C. for 1 h. Water (10 mL) was added and the mixture was allowed to warm to room temperature over 2 h. The layers were separated. The organic layer was washed with brine, dried (Na₂SO₄), and concentrated. The resulting residue was purified by silica gel chromatography eluting with 10% ethyl acetate in hexane to provide the desired product (1.735 g, 100%) as an orange oil.

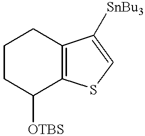

### EXAMPLE 260D

A solution of Example 260C (1.73 g, 4.99 mmol) in diethyl ether (10 mL) was cooled to −50° C., treated dropwise with n-butyl lithium (2.5 mL of a 2.5 M solution in hexanes, 6.25 mmol), stirred at −50° C. for 1.5 h. After cooling to −78° C. the reaction mixture was treated dropwise with a solution of chlorotributylstannane (1.65 mL, 5.1 mmol), allowed to warm to room temperature, and partitioned between ethyl acetate and water. The organic layer was washed with brine, dried (Na₂SO₄), and concentrated to give the desired product as a yellow oil that was used without fuirther purification.

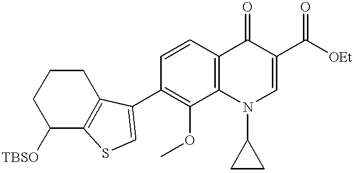

### EXAMPLE 260E

The desired product was prepared by substituting Example 260D and ethyl 7-bromo-1-cyclopropyl-8-methoxy-4-oxo-1,4-dihydro-3-quinolinecarboxylate for Example 1D and ethyl 7-bromo-1-cyclopropyl-4-oxo-1,4-dihydro-3-quinolinecarboxylate, resxpectively in Example 1E.

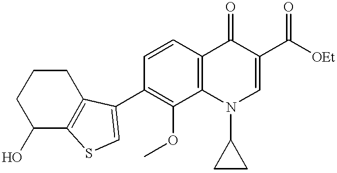

### EXAMPLE 260F

The desired product was prepared by substituting Example 260E for Example 35D in Example 35E and was purified by silica gel chromatography eluting with hexane then 50% acetone in hexane to provide the desired product.

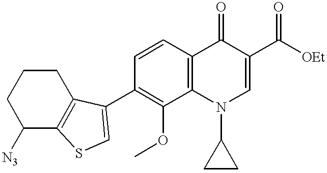

### Example 260G

The desired product was prepared by substituting Example 260F for Example 37A Example 37B.

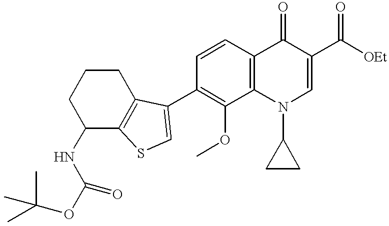

### EXAMPLE 260H

The desired product was prepared by substituting Example 260G for Example 201C in Example 201D.

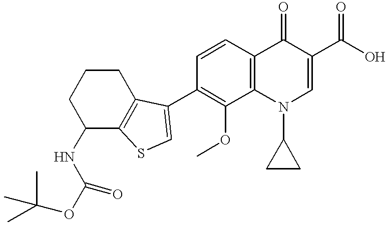

### EXAMPLE 260I

The desired product was prepared by substituting Example 260H for Example 2A in Example 2B.

### EXAMPLE 260J

### 7-(7-amino-4,5,6,7-tetrahydro-1-benzothien-3-yl)-1-cyclopropyl-8-methoxy-4-oxo-1,4-dihydro-3-guinolinecarboxvlic acid

The desired product was prepared by substituting Example 260I for Example 40B in Example 40C. MS (APCI) m/z 445 (M+Cl)⁻; ¹H NMR (300 MHz, DMSO-d₆) δ 8.82 (s, 1H), 8.38 (br s, 2 H), 8.17 (d, 1H), 7.79 (s, 1H), 7.48 (d, 1H), 4.63 (m, 1H), 4.23 (m, 1H), 3.42 (s, 3H), 3.15 (m, 2H), 2.25-1.75 (m, 4H), 1.17-1.10 (m, 4H).

### EXAMPLE 261

### 7-(7-amino-4,5,6,7-tetrahydro-1-benzothien-2-yl)-1-cyclopropyl-8-(difluoromethoxy)-4-oxo-1,4-dihydro-3-quinolinecarboxylic acid

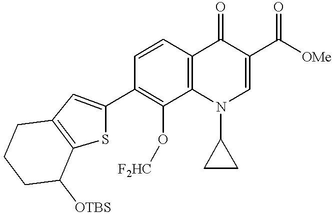

### EXAMPLE 261A

The desired product was prepared by substituting Example 41B and ethyl 7-bromo-1-cyclopropyl-8-difluoromethoxy-4-oxo-1,4-dihydro-3-quinolinecarboxylate for Example 1D and ethyl 7-bromo-1-cyclopropyl-4-oxo-1,4-dihydro-3-quinolinecarboxylate, respectively in Example 1E followed by substituting the crude product for Example 38C in Example 39A.

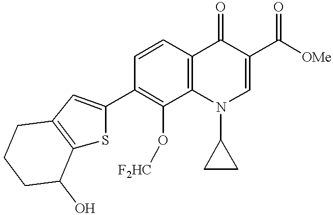

### EXAMPLE 261B

The desired product was prepared by substituting Example 261A for Example 35D in Example 35E and was purified by silica gel chromatography eluting with hexane then 50% acetone in hexane to provide the desired product.

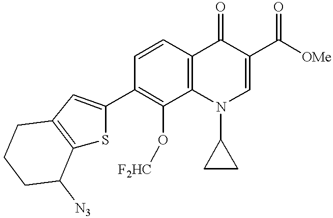

### EXAMPLE 261C

The desired product was prepared by substituting Example 261B for Example 37A in 37B.

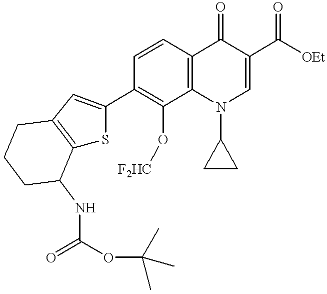

### EXAMPLE 261D

The desired product was prepared by substituting Example 261C for Example 201C in Example 201D.

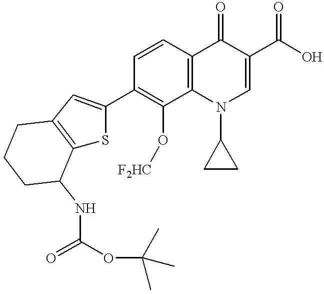

### EXAMPLE 261E

The desired product was prepared by substituting Example 261D for Example 2A in Example 2B.

### EXAMPLE 261F

### 7-(7-amino-4,5,6,7-tetrahydro-1-benzothien-2-yl)-1-cyclopropyl-8-(difluoromethoxy)-4-oxo-1,4-dihydro-3-quinolinecarboxylic acid

The desired product was prepared by substituting Example 261E for Example 40B in Example 40C. MS (DCI/NH3) m/z 447(M+1)⁺; ¹H NMR (300 MHz, DMSO-d₆) δ 14.58 (s, 1H), 8.88 (s, 1H), 8.49 (br s, 2H), 8.32 (d, 1H), 7.60 (s, 1H), 6.99 (dd, 1H), 4.63 (m, 1H), 4.15 (m, 1H), 2.68, (m, 2H) 2.14-1.82 (m 4H), 1.21-1.03 (m, 4H).

### EXAMPLE 262

### 1-cyclopropyl-7-(7-hydroxy-5,5-dimethyl-4,5,6,7-tetrahydro-1-benzothien-2-yl)-8-methoxy-4-oxo-1,4-dihydro-3-quinolinecarboxylic acid

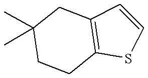

### EXAMPLE 262A

The desired product was prepared by substituting Example 213A for Example 185C in Example 185D.

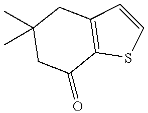

### EXAMPLE 262B

The desired product was prepared by substituting 262A for 4,4-dimethyl-4,5,6,7-tetrahydrobenzo[b]thiophene in Example 218A and was purified by silica gel chromatography eluting with CH₂Cl₂.

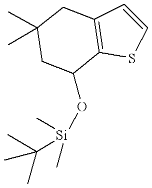

### EXAMPLE 262C

The desired product was prepared by substituting Exa mple 262B for Example 218A in Example 218B

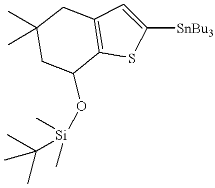

### EXAMPLE 262D

The desired product was prepared by substituting Example 262C for Example 218B in Example 218C.

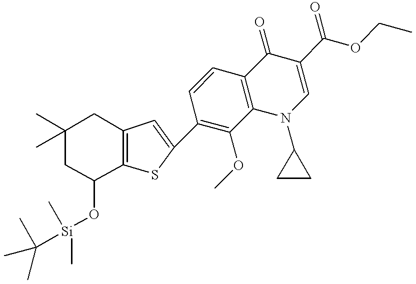

### EXAMPLE 262E

The desired product was prepared by substituting Example 262D, ethyl 7-bromo-1-cyclopropyl-8-methoxy-4-oxo-1,4-dihydro-3-quinolinecarboxylate and a reaction time of 8.5 hours for Example 1D, ethyl 7-bromo-1-cyclopropyl-4-oxo-1,4-dihydro-3-quinoline-3-carboxylate and a reaction time of 8.5 hours, respectively in Example 1E.

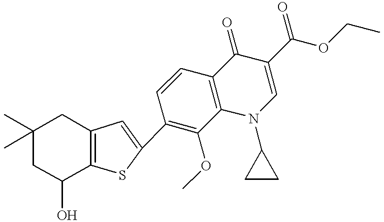

### EXAMPLE 262F

The desired product was prepared by substituting Example 262E for Example 35D in Example 35E and was purified by silica gel chromatography eluting with 3.5% MeOH in CH₂Cl₂.

### EXAMPLE 262G

### 1-cyclopropyl-7-(7-hydroxy-5,5-dimethyl-4,5,6,7-tetrahydro-1-benzothien-2-yl)-8-methoxy-4-oxo-1,4-dihydro-3-quinolinecarboxylic acid

The desired product was prepared by substituting Example 262F for Example 2A in Example 2B. mp 182-183° C.; MS (APCI) m/z 440 (M+H)⁺; ¹H NMR (300MHz, CDCl₃) δ 14.72 (br s, 1H), 8.91 (s, 1H), 8.23 (d, 1H), 7.78 (d, 1H), 7.33 (s, 1H), 5.00 (br m, 1H), 4.10 (m, 1H), 3.67 (s, 3H), 2.50 (dd, 2H), 2.04 (dd, 2H), 1.55 (br s, 1H), 1.25 (m, 2H), 1.15 (s, 3H), 1.08 (m, 2H), 1.02 (s, 3H).

### EXAMPLE 263

### 7-(7-amino-5,5 -dimethyl-4,5,6,7-tetrahydro-1-benzothien-2-yl)-1-cyclopropyl-8-methoxy-4-oxo-1,4-dihydro-3-quinolinecarboxylic acid hydrochloride

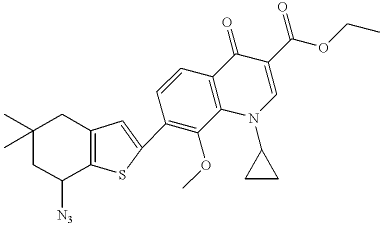

### EXAMPLE 263A

The desired product was prepared by substituting Example 262F for Example 37A in Example 37B and was purified by silica gel chromatography eluting with 2% MeOH in CH₂Cl₂.

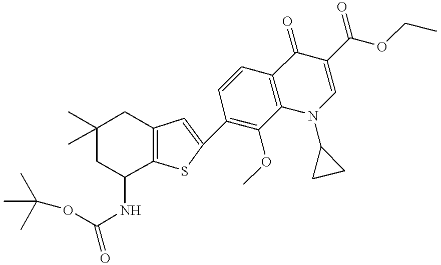

### EXAMPLE 263B

The desired product was prepared by substituting 263A for 201C in Example 201D and was purified by silica gel chromatography eluting with 2% MeOH in CH₂Cl₂.

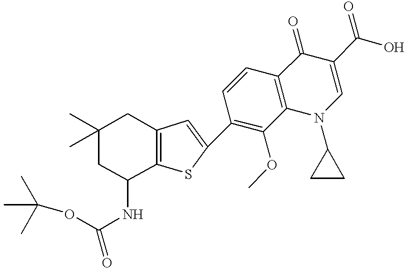

### EXAMPLE 263C

The desired product was prepared by substituting Example 263B for 2A in Example 2B.

### EXAMPLE 263D

### 7-(7-amino-5,5-dimethyl-4,5,6,7-tetrahydro-1-benzothien-2-yl)-1-cyclopropyl-8-methoxy-4-oxo-1,4-dihydro-3-quinolinecarboxylic acid hydrochloride

The desired product was prepared by substituting Example 263C for Example 40B in Example 40C. mp 194-195° C.; MS (APCI) m/z 439 (M+H)⁺; ¹H NMR (300MHz, CDCl₃) δ 8.83 (s, 1H), 8.52 (br s, 3H), 8.15 (d, 1H), 8.06 (d, 1H), 7.68 (s, 1H), 4.53 (m, 1H), 4.28 (m, 1H), 3.73 (s, 3H), 3.30 (s, 1H), 2.45 (m, 2H), 2.03 (, 2H), 1.16 (s, 3H), 1.12 (m, 2H), 1.08 (m, 21), 0.94 (s, 3H).

### EXAMPLE 264

### 1-cyclopropyl-7-(4-hydroxy-4,5,6,7-tetrahydro-1-benzofuran-2-yl)-8-methoxy-4-oxo-1,4-dihydro-3-quinolinecarboxylic acid

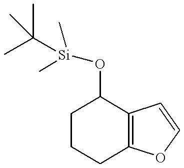

### EXAMPLE 264A

The desired product was prepared by substituting 4-hydroxy-4,5,6,7-tetrahydrobenzofuran (prepared by the method of L. Mase, et.al. Tetrahedron. 1996, 52, 8169-8180) for Example 185F in Example 185G.

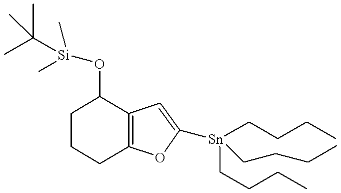

### EXAMPLE 264B

The desired product was prepared by substituting Example 264A for Example 218B in Example 218C.

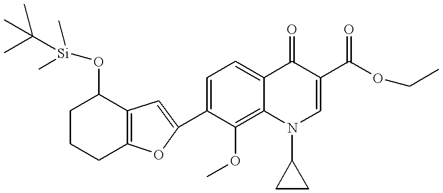

### EXAMPLE 264C

The desired product was prepared by substituting Example 264B and ethyl-7-bromo-1-cyclopropyl-8-methoxy-4-oxo-1,4-dihydro-3-quinolinecarboxylate for Example 1D and ethyl-7-bromo-1-cyclopropyl-4-oxo-1,4-dihydro-3-quinolinecarboxylate, respectively in Example 1E.

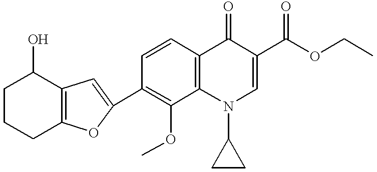

### EXAMPLE 264D

The desired product was prepared by substituting Example 264C for Example 35D in Example 35E.

### EXAMPLE 264E

### 1-cyclopropyl-7-(4-hydroxy-4,5,6,7-tetrahydro-1-benzofuran-2-yl)-8-methoxy-4-oxo-1,4-dihydro-3-quinolinecarboxvlic acid

The desired product was prepared by substituting Example 264D for Example 2A in Example 2B. MS (ESI) m/z 430 (M+Cl)⁻)⁻; ¹H NMR (300 MHz, DMSO-d₆) δ 14.94 (s, 1H), 8.78 (d,1H), 7.97(d, 1H), 7.26(s,1H), 5.05 (br s, 5.05), 4.62(m,1H), 4.26 (m,1H), 3.75 (s, 1H), 2.68-2.59 (m, 2H), 1.98 (m,1H), 1.88 (m,1H), 1.75 (m,1H), 1.67 (m, 1H), 1.17 (d, 2H), 1.04 (s, 2H).

### EXAMPLE 265

### 7-(4-amino-4,5,6,7-tetrahydro-1-benzofuran-2-yl)-1-cyclopropyl-8-methoxy-4-oxo-1,4-dihydro-3-quinolinecarboxylic acid hydrochloride

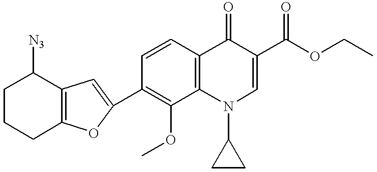

### EXAMPLE 265A

The desired product was prepared by substituting Example 264D for Example 37A in Example 37B and was purified by chromatography on silica gel eluting with CH₂Cl₂:CH₃OH (96:4).

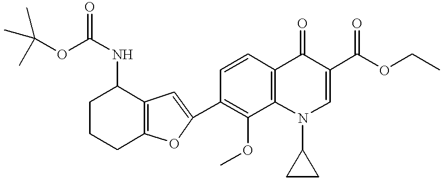

### EXAMPLE 265B

The desired product was prepared by substituting Example 265A for Example 201C in Example 201D and was purified by chromatography on silica gel eluting with CH₂Cl₂:CH₃OH (96:4).

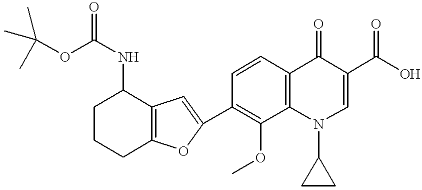

### EXAMPLE 265C

The desired product was prepared by substituting Example 265B for Example 2A in Example 2B.

### EXAMPLE 265D

### 7-(4-amino-4,5,6,7-tetrahydro-1-benzofuran-2-yl)-1-cyclopropyl-8-methoxy-4-oxo-1,4-dihydro-3-quinolinecarboxylic acid hydrochloride

The desired product was prepared by substituting Example 265C for Example 40B in Example 40C. MS (ESI) m/z 395 (M+H+)⁺; ¹H NMR (300 MHz, CD₃OD) δ 8.96 (s, 1H), 8.23 (d, 1H), 7.47 (s, 1H), 4.48 (m, 1H), 4.32 (m, 1H), 3.81 (s, 3H), 2.82 (m, 2H), 2.82-2.13 (m, 2H), 2.04-1.90 (m, 2H), 1.24 (d, 2H), 1.07 (s, 2H).

### EXAMPLE 266

### ethyl 7-bromo-1-cyclopropyl-6-fluoro-4-oxo-1,4-dihydroquinoline-3-carboxylate

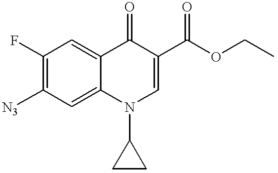

### EXAMPLE 266A

A solution of ethyl 1-cyclopropyl-6,7-difluoro-4-oxo-1,4-dihydro-quinoline-3-carboxylate (Sanchez, J. P., Domagala, J. M., Hagen, S. E., Heifetz, C. L., Hutt, M. P. J. Med. Chem. 1988, 31, 983-991.) (10.03 g, 0.0342 mol) and sodium azide (6.67 g, 0.1026 mol) in DMF (250 ml) was heated to 65° C. for 4 h, allowed to cool to room temperature and was poured into 700 ml water. The resulting heterogeneous mixture was filtered, the solid washed with water and dried in vacuo to provide the desired product as a colorless solid (10.25 g, 95%).

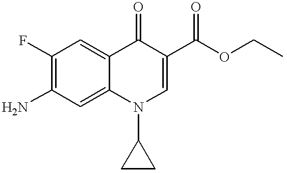

### EXAMPLE 266B

A solution of Example 266A (10.21 g, 0.0324 mol) in 1:1 MeOH:THF (700 ml) was treated with 10% Pd/C (0.510 g, 5 wt %) and was stirred at ambient temperature under 1 atm H₂ gas for 14 h. The resulting mixture was warmed to 45° C. in a water bath, filtered through Celite and the pad rinsed with CH₂Cl₂. Concentration gave a solid that was triturated in boiling hexanes and collected by filtration to provide the desired product as a yellow crystalline solid (9.12 g, 97%).

### EXAMPLE 266C

### ethyl 7-bromo-1-cyclopropyl-6-fluoro-4-oxo-1,4-dihydroquinoline-3-carboxylate

A suspension of Example 266B (9.10 g, 0.0314 mol) in 5% aqueous HBr (400 ml) was treated with cupric bromide (35.01 g, 0.157 mol), cooled to 0° C., treated dropwise with a solution of sodium nitrite (4.32 g, 0.0627 mol) in water (20 ml), was allowed to warm to room temperature and stir for 12 h. The reaction mixture was extracted with CH₂Cl₂ (4×100 ml), the combined organic layers washed with 5% HBr, water, brine and dried (MgSO₄). The resulting crude solid was recrystallized from acetone to provide the desired product as a colorless solid (7.26 g, 65%). MS (DCI/NH₃) m/z 354 (M+H)⁺; ¹H NMR (300MHz, DMSO-d₆) δ 8.48 (s, 1H), 8.40 (d, 1H), 7.98 (d, 1H), 4.23 (q, 2H), 3.69 m, 1H), 1.28 (t, 3H), 1.27 (m, 2H), 1.13 (m, 2H).

### EXAMPLE 267

### 7-(7-hydroxy-6-spirocyclopropyl-4,5,6,7-tetrahydro-1-benzothien-2-yl)-1-cyclopropyl-8-methoxy-4-oxo-1,4-dihydroquinoline-3-carboxylic acid

The desired product was prepared by substituting Example 270E for Example 296B in Example 296C. The reaction mixture was brought to pH 4 with 1M HCl giving a yellow powder which was collected by filtration and dried. MS (DCI) m/z 438 (M+H)⁺; ¹H NMR (300 MHz, DMSO-d6) δ 14.93 (s, 1H), 8.79 (s, 1H), 8.10 (d, 1H), 8.00 (d, 1H), 7.58 (s, 1H), 5.37 (d, 1H), 4.26 (m, 1H), 4.19 (d, 1H), 3.69 (s, 3H), 2.73-2.51 (m, 2H), 1.96 (m, 1H), 1.40 (m, 1H), 1.17-1.03 (m, 4H), 0.71 (m, 1H), 0.51 (m, 1H), 0.46 (m, 2H).

### EXAMPLE 268

### 7-(7-amino-5,5-dimethyl-4,5,6,7-tetrahydro-1-benzothien-2-yl)-1-cyclopropyl-8-methoxy-4-oxo-1,4-dihydrocquinoline-3-carboxylic acid hydrochloride

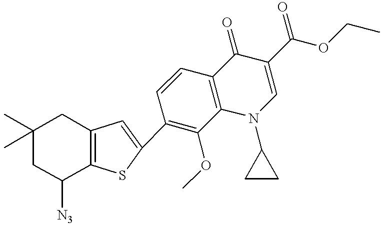

### EXAMPLE 268A

The desired product was prepared by substituting Example 262F for Example 37A in Example 37B and was purified by silica gel chromatography eluting with 2% MeOH in CH₂Cl₂.

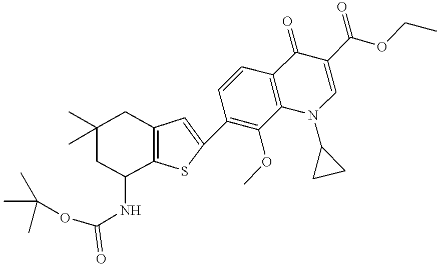

### EXAMPLE 268B

The desired product was prepared by substituting 268A for 201C in Example 201D and was purified by silica gel chromatography eluting with 2% MeOH in CH₂Cl₂.

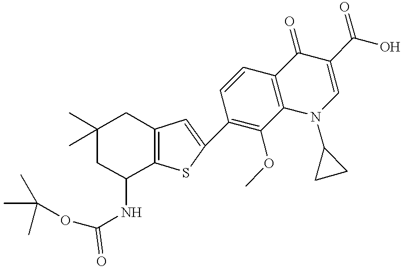

### EXAMPLE 268C

The desired product was prepared by substituting Example 268B for 2A in Example 2B.

### EXAMPLE 268D

### 7-(7-amino-5,5-dimethyl-4,5,6,7-tetrahydro-1-benzothien-2-yl)-1-cyclopropyl-8-methoxy-4-oxo-1,4-dihydroquinoline-3-carboxylic acid hydrochloride

The desired product was prepared by substituting Example 268C for 40B in Example 40C. mp 194-195° C.; MS (APCI) m/z 439 (M+H)⁺; ¹H NMR (300MHz, CDCl₃) δ 8.83 (s, 1H), 8.52 (br s, 3H), 8.15 (d, 1H), 8.06 (d, 1H), 7.68 (s, 1H), 4.53 (m, 1H), 4.28 (m, 1H), 3.73 (s, 3H), 3.30 (s, 1H), 2.04 (m, 2H), 1.64 (, 2H), 1.16 (s, 3H), 1.12 (m, 2H), 1.08 (m, 2H), 0.94 (s, 3H).

### EXAMPLE 269

### ethyl 7-bromo-1-cyclopropyl-6-methyl-4-oxo-1,4-dihydroquinoline-3-carboxylate

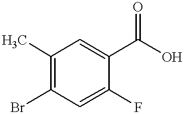

### EXAMPLE 269A

A solution 5.25% sodium hypochlorite (200 mL) and sodium hydroxide (5.8 g, 0.15 mol) was cooled to 0° C. and treated with a solution of 1-(4-bromo-2-fluoro-5-methyl-phenyl)-ethanone (prepared by the method of Hamper, Leschinsky WO9602486) (11.12 g, 48.10 mmol) in dioxane (10 mL). The resulting mixture was stirred at ambient temperature for 20 h, sodium bisulfite (25 g, 0.15 mol) was added to the solution and after 20 minutes the solution was diluted with water (200 mL) and extracted with dichloromethane. The aqueous layer was adjusted to pH 2 with conc HCl, and was extracted with dichloromethane. The organic phase was dried (Na₂SO₄), and concentrated to yield desired product (4.66 g, 42%).

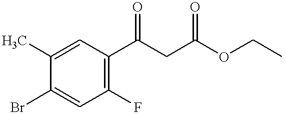

### EXAMPLE 269B

A solution of Example 269A (5.75 g, 24.70 mmol) in dichloromethane (60 mL) and N,N-dimethylformamide (3 drops) was cooled to 0° C., treated with oxalyl chloride (3.29 mL, 37.00 mmol) and stirred at ambient temperature for 2.5 h. The reaction mixture was concentrated under vacuum, the resulting residue dissolved in acetonitrile (10 mL) and added dropwise to a mixture of the potassium salt of ethyl malonate (8.41 g, 49.40 mmol), triethyl amine (13.77 mL, 98.80 mmol), and magnesium chloride (5.88 g, 61.80 mmol) at 0° C. and stirred at ambient temperature for 3 h. The reaction mixture was concentrated under vacuum, the residue diluted with water, adjusted to pH 2 with conc HCl and extracted with dichloromethane. The organic phase was dried (Na₂SO₄), and concentrated to yield the desired product (7.61 g, 100%) which was used without purification.

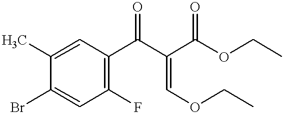

### EXAMPLE 269C

A neat solution of Example 269B (7.48 g, 24.70 mmol) in acetic anhydride (9.31 mL, 98.80 mmol) and triethyl orthoformate (6.16 mL, 37.00 mmol) was heated at 120° C. for 3 h. The reaction mixture was concentrated under vacuum to yield the desired product (8.86 g, 100%) that was used without purification.

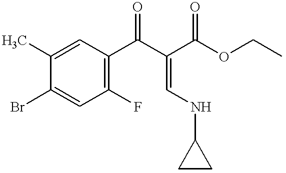

### EXAMPLE 269D

A solution of Example 269C (8.86 g, 24.70 mmol) in dichloromethane (120 mL) was cooled to 0° C., treated with cyclopropylamine (2.39 mL, 34.50 mmol), stirred at ambient temperature for 3 h and diluted with water (250 mL). The reaction mixture was extracted with dichloromethane, the organic phase dried (Na₂SO₄)and concentrated to yield the desired product (9.13 g, 100%) as an oil which was used without purification.

### EXAMPLE 269E

### ethyl 7-bromo-1-cyclopropyl-6-methyl-4-oxo-1,4-dihydroquinoline-3-carboxylate

A solution of Example 269D (9.13 g, 24.7 mmol) in N, N-dimethylformamide (50 mL) was treated with potassium carbonate (5.11 g, 37.00 mmol) and heated at 80° C. for 3 h, allowed to cool to ambient temperature and was diluted with water. The reaction mixture was extracted with dichloromethane, dried (Na₂SO₄) and concentrated. The resulting solid was triturated in hexane, filtered, and dried to yield the desired compound (2.82 g, 33%) as a dark tan solid. MS (DCI/NH₃) mn/z 352 (M+H)⁺¹H NMR (300MHz, CDCl3) δ 8.54 (s, 1H), 8.32 (s, 1H), 8.11 (s, 1H), 4.39 (q, 2H), 3.44 (m, 1H), 2.51 (s, 31H), 1.42 (t, 3H), 1.44 (m, 2H), 1.14 (m, 2H)

### EXAMPLE 270

### 7-(6-(2-aminoethyl)-4,5,6,7-tetrahydro-1-benzothien-2-yl)-1-cyclopropyl-8-methoxy-4-oxo-1,4-dihydroquinoline-3-carboxylic acid hydrochloride

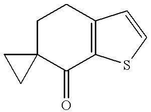

### EXAMPLE 270A

A stirred solution of potassium tert-butoxide (IM in tert-butanol, 90 mL, 90 mmol) was treated with 5,6-dihydrobenzo[b]thiophen-7(4H)-one, (J. P. Conjat, P. Cagniant, D. Cagniant, M. Mirjolet; Tetrahedron Letters, 1975, 2885-2888, 6.84 g, 45 mmol) portionwise, sodium iodide (1.35 g, 9 mmol) and stirred for 15 minutes. To this mixture was added 2-chloroethyl dimethyl sulfonium iodide, (S. Ruder and R. Ronald; Tetrahedron Letters, 1984, 25, 5501-5504, 11.34 g, 45 mmol) in single gram portions over 1 hour. The resulting mixture was stirred for 48 hours, diluted with EtOAc and saturated ammonium chloride (300 mL each) and carefully acidified to pH 4 with 1M HCl. The aqueous layer was extracted 3×100 mL with EtOAc. The extracts were combined, washed with brine, dried (MgSO₄) and concentrated. The crude product was purified by silica gel chromatography eluting with 4:1 hexane/EtOAc to provide the desired compound as a yellow oil (1.4 g, 17%).

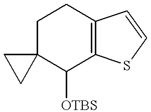

### EXAMPLE 270B

The desired product was prepared by substituting Example 270A for 2-bromo-4-keto-4,5,6,7-tetrahydrothianapthene in Example 35A and the crude product was used without purification.

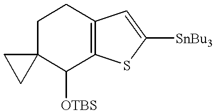

### EXAMPLE 270C

The desired product was prepared by substituting Example 270B for Example 11C in Example 11D and was used without purification.

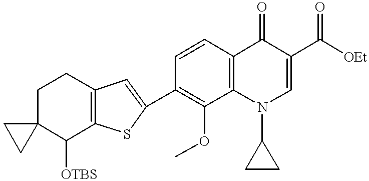

### EXAMPLE 270D

The desired product was prepared by substituting ethyl 7-bromo-1-cyclopropyl-8-methoxy-4-oxo-1,4-dihydro-3-quinolinecarboxylate and Example 270C for Example 266 and Example 38B, respectively, in Example 296A and was purified by silica gel chromatography eluting with 2:1 hexane/acetone to provide the desired compound as an off-white solid.

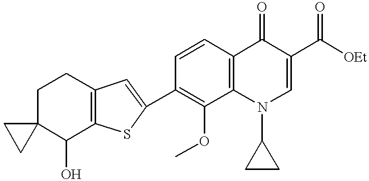

### EXAMPLE 270E

The desired product was prepared by substituting Example 270D for Example 35D in Example 35E and was used without purification.

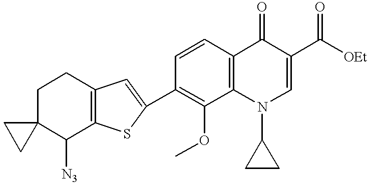

### EXAMPLE 270F

The desired product was prepared by substituting Example 270E for Example 37A in Example 37B using dichloromethane as the reaction solvent and extraction solvent. The crude product was purified by silica gel chromatography eluting with 2:1 hexane/acetone to provide the desired compound as a white solid.

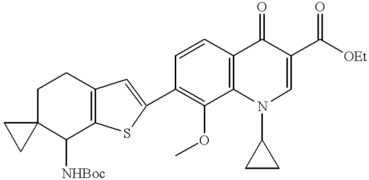

### EXAMPLE 270G

The desired product was prepared by substituting Example 270F for Example 201C in Example 201D and was purified by silica gel chromatography eluting with 1:1 hexane/acetone to provide the desired compound as a white solid.

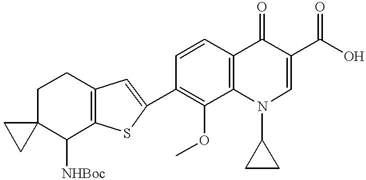

### EXAMPLE 270H

The desired product was prepared by substituting Example 270G for Example 296B in Example 296C.

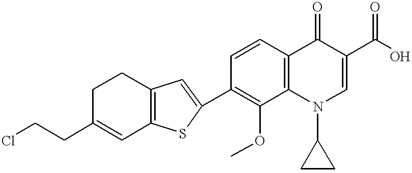

### EXAMPLE 270I

The desired product was prepared by substituting Example 270H for Example 40B in Example 40C and was triturated in Et₂O and the red/orange solid collected by filtration.

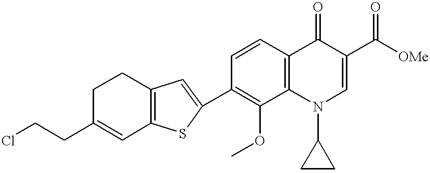

### EXAMPLE 270J

The desired product was prepared as a yellow powder by substituting Example 270I for Example 38C in Example 39A and was triturated in Et₂O and the solid collected by filtration.

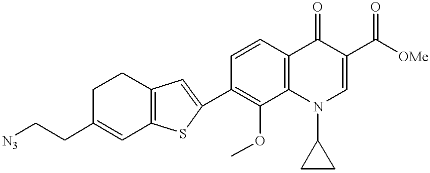

### EXAMPLE 270K

A solution of Example 270J (94 mg, 0.2 mmol) and NaN₃ (65 mg, 1.0 mmol) in DMSO (3 mL) was heated at 55° C. for 5 hours, cooled, and partitioned between EtOAc and water. The EtOAc layer was washed with brine, dried (Na₂SO₄) and concentrated to give the desired material (44 mg, 88%) that was used without purification.

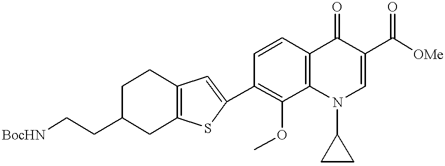

### EXAMPLE 270L

The desired product was prepared as a yellow oil by substituting Example 270K for Example 201C in Example 201D and was purified by silica gel chromatography eluting with 1:2 hexane/acetone.

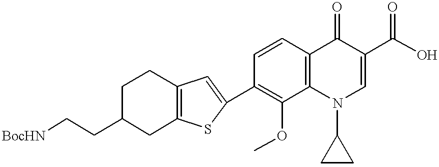

### EXAMPLE 270M

The desired product was prepared by substituting Example 270L for Example 296B in Example 296C and the resulting yellow solid was used without purification.

### EXAMPLE 270N

### 7-(6-(2-aminoethyl)-4,5,6,7-tetrahydro-1-benzothien-2-yl)-1-cyclopronyl-8-methoxy-4-oxo-1,4-dihydroquinoline-3-carboxylic acid hydrochloride

The desired product was prepared as a bright yellow solid by substituting Example 270M for Example 40B in Example 40C. MS (DCI) m/z 439 (M+H)⁺; ¹H NMR (300 MHz, DMSO-d6) δ 8.79 (s, 1H), 8.09 (d, 1H), 7.98 (d, 1H), 7.87 (bs, 3H), 7.58 (s, 1H), 4.26 (m, 1H), 3.70 (m, 2H), 3.63 (s, 3H), 3.00-2.55 (m, 4H), 1.91 (m, 2H), 1.65 (m, 2H), 1.17-1.03 (m, 4H).

### EXAMPLE 271

### 7-(7-amino-4,5,6,7-tetrahydro-1-benzothien-2-yl)-1-cyclopropyl-6-fluoro-4-oxo-1,4-dihydro-1,8-naphthyridine-3-carboxylic acid hydrochloride

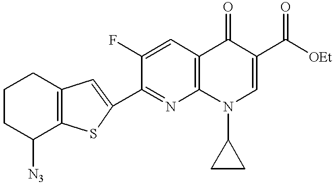

### EXAMPLE 271A

The desired product was prepared by substituting Example 320B for Example 37A in Example 37B using dichloromethane as the reaction solvent and was purified by trituration in 4:1 hexane/acetone and the solid collected by filtration.

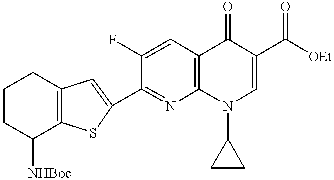

### EXAMPLE 271B

The desired product was prepared by substituting Example 271A for Example 201C in Example 201D and was purified by silica gel chromatography eluting with 95:5 dichloromethane/methanol to provide the desired compound.

### EXAMPLE 271C

### 7-(7-amino-4,5,6,7-tetrahydro-1-benzothien-2-yl)-1-cyclopropyl-6-fluoro-4-oxo-1,4-dihydro-1,8-naphthyridine-3-carboxylic acid hydrochloride

The desired product was prepared as a yellow powder by substituting Example 271B and THF for Example 48C and EtOH, respectively, in Example 48D. MS (APCI) m/z 400 (M+H)⁺; ¹H NMR (300 MHz, DMSO-d6) δ 8.84 (s, 1H), 8.59 (d, 1H), 8.51 (bs, 3H), 7.85 (d, 1H), 4.67 (m, 1H), 3.82 (m, 1H), 2.72 (m, 2H), 2.19-1.74 (m, 4H), 1.35-1.18 (m, 4H).

### EXAMPLE 272

### 1-(2,4-difluorophenyl)-6-fluoro-7-(7-hydroxy-4,5,67-tetrahydro-1-benzothien-2-yl)-4-oxo-1,4-dihydro-1,8-naphthyridine-3-carboxylic acid

### EXAMPLE 272B

The desired compound was prepared by substituting Example 272A in Example 277A for Example 35D in Example 35E and was purified by trituration with 1:1 hexane:diethyl ether to yield a yellow solid. MS (DCI/NH₃) m/z 473 (M+H)⁺¹H NMR (300MHz, CDCl3) δ 14.15 (s, 1H), 8.87 (2, 1H), 8.44 (d, 1H), 7.62 (d, 1H), 7.45 (m, 1H), 7.15 (m, 2H), 4.89 (m, 1H), 2.66 (m, 2H), 1.74-2.16 (m, 4H).

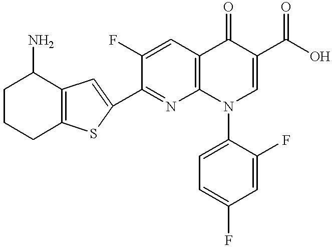

### EXAMPLE 273

### 7-(4-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid hydrochloride

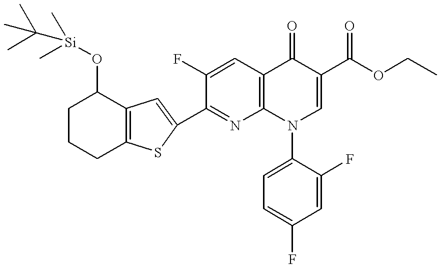

### EXAMPLE 273A

The desired compound was prepared by substituting Ethyl 7-chloro-1-(2,4-difluoro-phenyl)-6-fluoro-4-oxo-1,4-dihydro-[1,8]naphthyridine-3-carboxylate (prepared by the method of Chu, Fernades, et. al. J. Med. Chem. 1986, 29, 2363-2369) and Example 38B for ethyl 7-bromo-1-cyclopropyl-4-oxo- 1,4-dihydro-3-quinolone-3-carboxylate and Example 1D, respectively, in Example 1E with a reaction time of 4h and was purified by silica gel chromatography eluting with 3% methanol, 0.5% ammonium hydroxide/dichloromethane to yield a yellow solid.

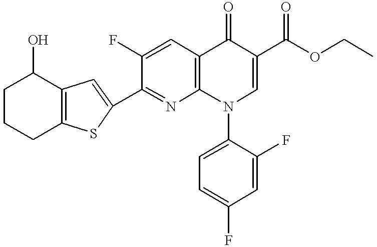

### EXAMPLE 273B

The desired compound was prepared by substituting Example 273A for Example 35D in Example 35E and was used without purification.

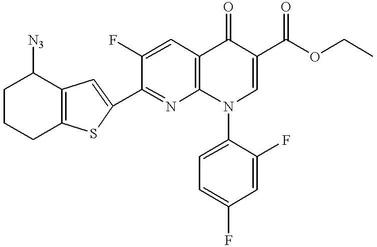

### EXAMPLE 273C

The desired compound was prepared by substituting Example 273B for Example 37A in Example 37B and was purified by silica gel chromatography eluting with 2% methanol/dichloromethane to yield a yellow solid.

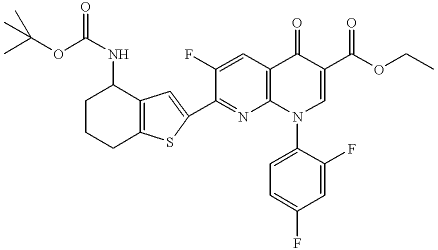

### EXAMPLE 273D

The desired compound was prepared by substituting Example 273C for Example 201C in Example 201D and was purified with silica gel chromatography eluting with 2% methanol/dichloromethane to yield a yellow solid.

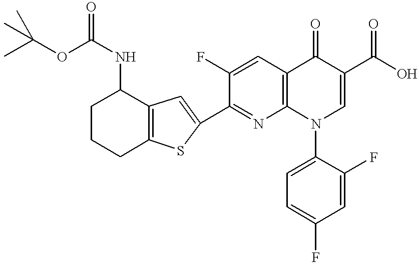

### EXAMPLE 273E

The desired compound was prepared by substituting Example 273D for Example 2A in Example 2B to yield a yellow solid that was used without purification.

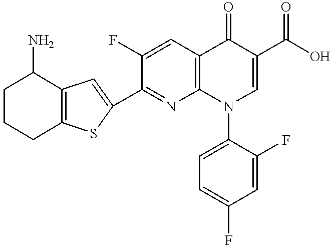

### EXAMPLE 273F

### 7-(4-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid hydrochloride

The desired compound was prepared by substituting Example 273E for Example 40B in Example 40C and was purified by trituration with diethyl ether to yield a yellow solid. MS (DCI/NH₃) m/z 472 (M+H)⁺¹H NMR (300 MHz, DMSO) δ 9.07 (s, 1H), 8.65 (d, 1H), 8.27 (m, 2H), 8.10 (m, 1H), 7.87 (m, 1H), 7.67 (m, 1H), 7.40 (m, 1H), 4.42 (m, 1H), 2.80 (m, 2H), 1.73-2.12 (m, 4H).

### EXAMPLE 274

### 1-(2,4-difluorophenyl)-6-fluoro-7-(4-hydroxy-4,5,6,7-tetrahydro-1-benzothien-2-yl)-4-oxo-1,4-dihydro-1,8-naphthyridine-3-carboxylic acid

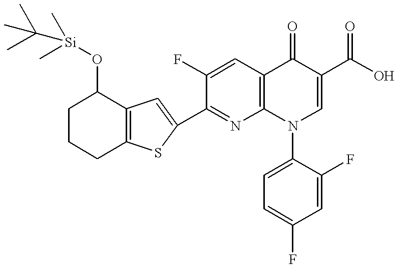

### EXAMPLE 274A

The desired compound was prepared by substituting Example 273A for Example 2A in Example 2B to yield a yellow oil that was used without purification.

### EXAMPLE 274B

### 1-(2,4-difluorophenyl)-6-fluoro-7-(4-hydroxy-4,5,6,7-tetrahydro-1-benzothien-2-yl)-4-oxo-1,4-dihydro-1,8-naphthyridine-3-carboxylic acid

The desired compound was prepared by substituting Example 274A for Example 35D in Example 35E and was purified by trituration with diethyl ether to yield a yellow solid. MS (DCI/NH₃) ni/z 473 (M+H)⁺¹H NMR (300 MHz, DMSO) δ 9.05 (s, 1H), 8.59 (d, 1H), 7.86 (m, 1H), 7.84 (d, 1H), 7.67 (m, 1H), 7.40 (m, 1H), 5.26 (m, 1H), 4.57 (m, 1H), 2.62 (m, 2H), 1.52-1.98 (m, 4H).

### EXAMPLE 275

### 1-(2,4-difluorophenyl)-6-fluoro-7-(4-(methylamino)-4,5,6,7-tetrahydro-1-benzothien-2-yl)-4-oxo-1,4-dihydro-1,8-naphthyridine-3-carboxulic acid hydrochloride

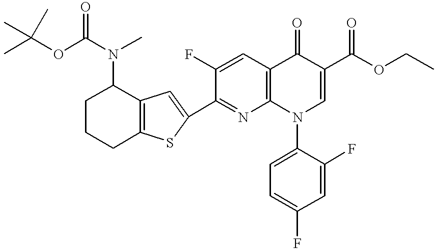

### EXAMPLE 275A

The desired compound was prepared by substituting ethyl 7-chloro-1-(2,4-difluoro-phenyl)-6-fluoro-4-oxo-1,4-dihydro-[1,8]naphthyridine-3-carboxylate (prepared by the method of Chu, Fernades, et. al. J. Med. Chem. 1986, 29, 2363-2369) and Example 210B for ethyl 7-bromo-1-cyclopropyl-4-oxo-1,4-dihydro-3-quinolone-3-carboxylate and Example 1D, respectively, in Example 1E with a reaction time of 8 h and was purified by silica gel chromatography eluting with 3% methanol, 0.5% ammonium hydroxide/dichloromethane to yield a dark yellow solid.

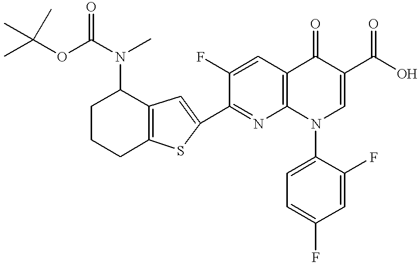

### EXAMPLE 275B

The desired compound was prepared by substituting Example 275A for Example 2A in Example 2B to yield a yellow solid that was used without purification.

### EXAMPLE 275C

### 1-(2,4-difluorophenyl)-6-fluoro-7-(4-(methylamino)-4,5,6,7-tetrahydro-1-benzothien-2-yl)-4-oxo-1,4-dihydro-1,8-naphthyridine-3-carboxylic acid hydrochloride

The desired compound was prepared by substituting Example 275B for Example 40B in Example 40C and was purified by trituration with diethyl ether to yield a yellow solid. MS (DCI/NH₃) m/z 486 (M+H)⁺¹H NMR (300 MHz, DMSO) δ 9.07 (s, 1H), 8.65 (d, 1H), 8.15 (d, 1H), 7.87 (m, 1H), 7.68 (m, 1H), 7.40 (m, 1H), 4.41 (m, 1H), 2.82 (m, 1H), 2.59 (m, 3H), 1.72-2.08 (m, 4H).

### EXAMPLE 276

### ethyl 7-bromo-1-(2,4-difluorophenyl)-6-fluoro-8-methoxy-4-oxo-1,4-dihydroquinoline-3-carboxylate

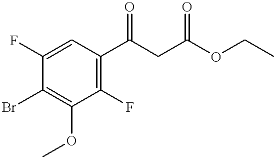

### EXAMPLE 276A

The desired product was prepared by substituting 4-bromo-2,5-difluoro-3-methoxy benzoic acid (prepared by the method of Todo, Hayashi, et. al. WO9605192) for Example 269A in Example 269B to yield a white solid.

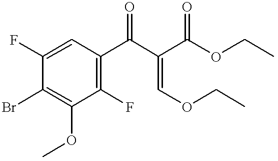

### EXAMPLE 276B

The desired product was prepared by substituting Example 276A for Example 269B in Example 269C to yield an oil that was used without purification.

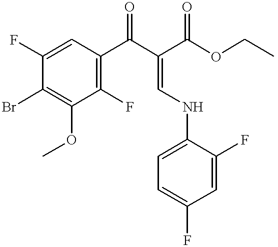

### EXAMPLE 276C

The desired product was prepared by substituting Example 276B and 2,4-difluoroaniline for Example 269C and cyclopropylamine, respectively, in Example 269D to yield a tan solid that was used without purification.

### EXAMPLE 276D

### ethyl 7-bromo-1-(2,4-difluorophenyl)-6-fluoro-8-methoxy-4-oxo-1,4-dihydroquinoline-3-carboxylate

The desired product was prepared by substituting Example 276C for Example 269D in Example 269E to yield a tan solid. MS (DCI/NH₃) m/z 457 (M+H)⁺¹H NMR (300MHz, CDCl₃) δ 8.28 (s, 1H), 8.12 (d, 1H), 7.47 (m, 1H), 7.05 (m, 2H), 4.40 (q, 2H), 3.33 (s, 3H), 1.41 (t, 3H).

### EXAMPLE 277

### 7-(7-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid hydrochloride

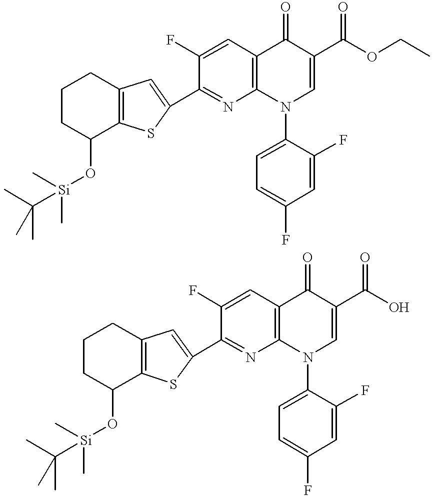

### EXAMPLE 277A EXAMPLE 272A

The desired products were prepared by substituting ethyl 7-chloro-1-(2,4-difluoro-phenyl)-6-fluoro-4-oxo-1,4-dihydro-[1,8]naphthyridine-3-carboxylate (prepared by the method of Chu, Fernades, et. al. J. Med. Chem. 1986, 29, 2363-2369) and Example 41B for ethyl 7-bromo-1-cyclopropyl-4-0×0-1,4-dihydro-3-quinolone-3-carboxylate and Example 1D, respectively, in Example 1E with a reaction time of 3h and was purified by silica gel chromatography eluting with 3% methanol, 0.5% ammonium hydroxide/dichloromethane.

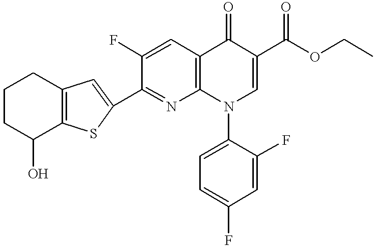

### EXAMPLE 277B

The desired compound was prepared by substituting Example 277A for Example 35D in Example 35E and was used without purification.

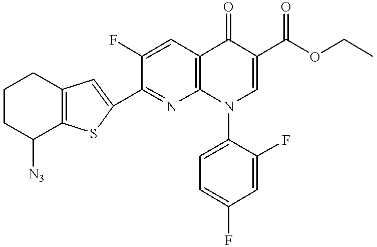

### EXAMPLE 277C

The desired compound was prepared by substituting Example 277B for Example 37A in Example 37B and was purified by silica gel chromatography eluting with 2% methanol/dichloromethane to yield a yellow solid.

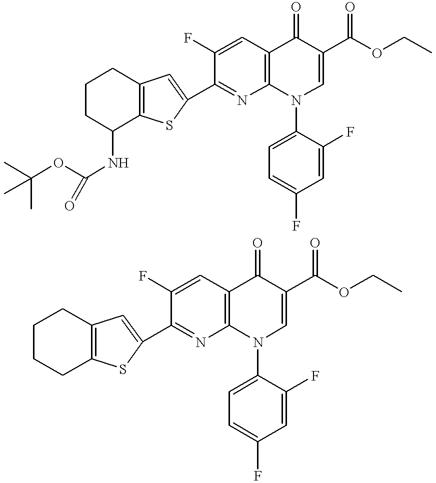

### EXAMPLE 277D EXAMPLE 286A

The desired products were prepared by substituting Example 277C for Example 201C in Example 201D and purified with silica gel chromatography eluting with 2% methanol/dichloromethane.

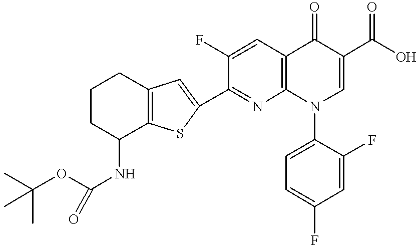

### EXAMPLE 277E

The desired compound was prepared by substituting Example 277D for Example 2A in Example 2B to yield a yellow solid that was used without purification.

### EXAMPLE 277F

### 7-(7-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid hydrochloride

The desired compound was prepared by substituting Example 277D for Example 40B in Example 40C and was purified by trituration with diethyl ether to yield a yellow solid. MS (DCI/NH₃) m/z 472 (M+H)⁺¹H NMR (300 MHz, DMSO) δ 9.06 (s, 1H), 8.67 (d, 1H), 8.38 (m, 2H), 7.87 (m, 1H), 7.70 (d, 1H), 7.62 (m, 1H), 4.58 (m, 1H), 2.65 (m, 2H), 1.70-2.12 (m, 4H).

### EXAMPLE 278

### 1-(2,4-difluorophenyl)-6-fluoro-8-methoxy-7-(4-(methylamino)-4,5,6,7-tetrahydro-1-benzothien-2-yl)-4-oxo-1,4-dihydroquinoline-3-carboxylic acid hydrochloride

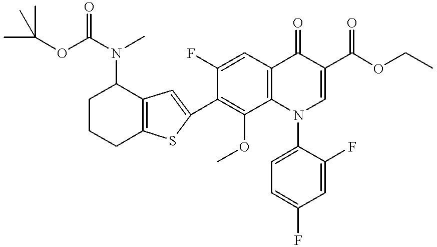

### EXAMPLE 278A

The desired product was prepared by substituting Example 276D and Example 210B for ethyl 7-bromo-1-cyclopropyl-4-oxo-1,4-dihydro-3-quinolone-3-carboxylate and Example 1D, respectively, in Example 1E with a reaction time of 8h and was purified by trituration in hexane to yield a yellow solid.

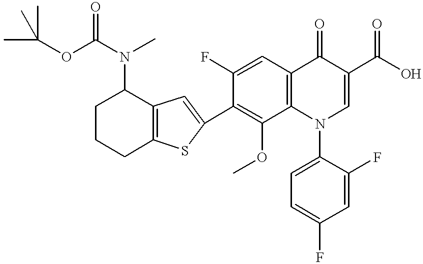

### EXAMPLE 278B

The desired compound was prepared by substituting Example 278A for Example 2A in Example 2B to yield a yellow solid that was used without purification.

### EXAMPLE 278C

### 1-(2,4-difluoropheyl-6-fluoro-8-methoxy-7-(4-(methylamino)-4,5,6,7-tetrahydro-1-benzothien-2-yl)-4-oxo-1,4-dihydroquinoline-3-carboxylic acid hydrochloride

The desired compound was prepared by substituting Example 278B for Example 40B in Example 40C and was purified by trituration with 2:1 diethyl ether:ethanol to yield a yellow solid. MS (DCI/NH₃) m/z 515 (M+H)⁺¹H NMR (300 MHz, DMSO) δ 8.70 (s, 1H), 8.07 (d, 1H), 8.03 (m, 1H), 7.73 (s, 1H), 7.58 (m, 1H), 7.34 (m, 1H), 4.41 (m, 1H), 3.00 (d, 3H), 2.84 (m, 2H), 2.59 (m, 3H), 2.03 (m, 3H), 1.83 (m, 1H).

### EXAMPLE 279

### 1-cyclopropyl-7-(4-(methylamino)-4,5,6,7-tetrahydro-1-benzothien-2-yl)-4-oxo-1,4-dihydroquinoline-3-carboxylic acid hydrochloride

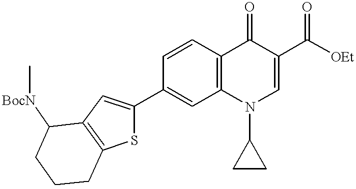

### EXAMPLE 279A

The desired product was prepared by substituting Example 210B for Example 1D and a reaction time of 8 h for 24 h in Example 1E and was purified by silica gel chromatography eluting with a gradient of 2:1 to 1:1 hexane/acetone to yield a yellow solid.

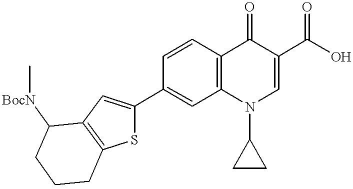

### EXAMPLE 279B

The desired product was prepared by substituting Example 279A for Example 2A and a reaction time of 3 h for an overnight reaction time in Example 2B to yield a yellow solid.

### EXAMPLE 279C

### 1-cyclopropyl-7-(4-(methylamino)-4,5,6,7-tetrahydro-1-benzothien-2-yl)-4-oxo-1,4-dihydroquinoline-3-carboxylic acid hydrochloride

The desired product was prepared by substituting Example 279B for Example 40B in Example 40C to yield a yellow solid. MS (DCI/NH₃) m/z 395 (M+H)⁺; ¹H NMR (300 MHz, DMSO-d6) δ 8.77 (s, 1H), 8.42 (d, 1H), 8.37 (d, 1H), 8.16 (s, 1H), 7.88 (dd, 1H), 4.41 (m, 1H), 3.90 (m, 1H), 2.88 (m, 2H), 2.65 (m, 3H), 2.10-1.80 (m, 4H), 1.43-1.07 (m, 4H).

### EXAMPLE 280

### 1-cyclopropyl-7-(7-hydroxy-4,5,6,7-tetrahydro-1-benzothien-2-yl)-4-oxo-1,4-dihydroquinoline-3-carboxylic acid

The desired product was prepared by substituting Example 288B for Example 2A and a reaction time of 2 h for an overnight reaction time in Example 2B to yield a yellow solid. MS (DCI/NH₃) m/z 382 (M+H)⁺; ¹H NMR (300 MHz, CDCl₃) δ 14.87 (s, 1H), 8.87 (s, 1H), 8.48 (d, 1H), 8.19 (d, 1H), 7.78 (dd, 1H), 7.22 (s, 1H), 4.96 (m, 1H), 3.62 (m, 1H), 2.77-2.65 (m, 21), 2.17-1.85 (m, 4H), 1.51-1.21 (m, 4H).

### EXAMPLE 281

### 1-cyclopropyl-7-(4-hydroxy-4,5,6,7-tetrahydro-1-benzothien-2-yl)-4-oxo-1,4-dihydroquinoline-3-carboxylic acid

The desired product was prepared by substituting Example 287B for Example 2A and a reaction time of 2.5 h for an overnight reaction time in Example 2B to yield a pale orange solid. MS (DCI/NH₃) m/z 382 (M+H)⁺; ¹H NMR (300 MHz, CDCl₃) δ 8.87 (s, 1H), 8.48 (d, 1H), 8.18 (d, 1H), 7.77 (dd, 1H), 7.50 (s, 1H), 4.83 (m, 1H), 3.62 (m, 1H), 2.90-2.75 (m, 2H), 2.12-1.84 (m, 4H), 1.51-1.23 (m, 4H).

### EXAMPLE 282

### 7-(7-amino-4,5,6,7-tetrahydro-1-benzothien-2-yl)-1-cyclopropyl-4-oxo-1,4-dihydro-1,8-naphthyridine-3-carboxylic acid hydrochloride

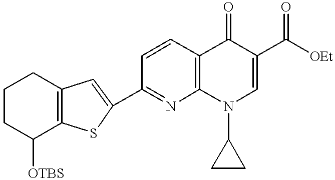

### EXAMPLE 282A

The desired product was prepared by substituting ethyl 1-cyclopropyl-7-chloro-4-oxo-1,4-dihydro[1,8]naphthyridine-3-carboxylate and Example 41B for Example 266 and Example 38B, respectively, in Example 296A and was purified by trituration with 3:1 hexane/acetone and the gray solid collected by filtration.

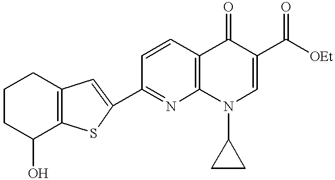

### EXAMPLE 282B

The desired product was prepared by substituting Example 282A for Example 35D in Example 35E and was used without purification.

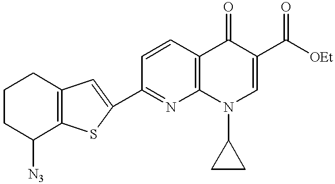

### EXAMPLE 282C

The desired product was prepared by substituting Example 282B for Example 37A in Example 37B using dichloromethane as both the reaction and extraction solvent and was purified by silica gel chromatography eluting with 1:1 hexane/acetone to provide the desired compound.

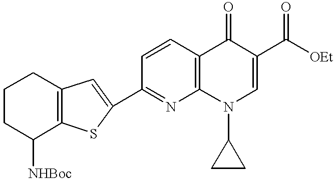

### EXAMPLE 282D

The desired product was prepared by substituting Example 282C for Example 201C in Example 201D and was purified by silica gel chromatography eluting with 2:1 hexane/acetone to provide the desired compound.

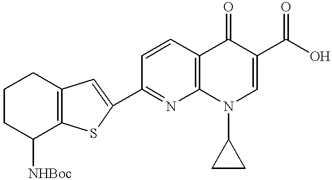

### EXAMPLE 282E

The desired product was prepared by substituting Example 282D for Example 296B in Example 296C.

### EXAMPLE 282F

### 7-(7-amino-4,5,6,7-tetrahydro-1-benzothien-2-yl)-1-cyclopropyl-4-oxo-1,4-dihydro-1,8-naphthyridine-3-carboxylic acid hydrochloride

The desired product was prepared as a yellow solid by substituting Example 282E for Example 40B in Example 40C. MS (APCI) m/z 382 (M+H)⁺; ¹H NMR (300 MHz, DMSO-d₆) δ 8.83 (s, 1H), 8.70 (d, 1H), 8.45 (bs, 3H), 8.20 (d, 1H), 7.96 (s, 1H), 4.63 (m, 1H), 3.82 (m, 1H), 2.69 (m, 2H), 2.18-1.72 (m, 4H), 1.30-1.18 (m, 4H).

### EXAMPLE 283

### 1-(2,4-difluorophenyl)-6-fluoro-7-(4-hydroxy-4,5,6,7-tetrahydro-1-benzothien-2-yl)-8-methoxy-4-oxo-1,4-dihydroquinoline-3-carboxylic acid

The desired compound was prepared by substituting Example 305B for Example 2A in Example 2B and was purified by trituration with 1:1 hexane:diethyl ether to yield a yellow solid. MS (DCI/NH₃) m/z 502 (M+H)⁺¹H NMR (300 MHz, DMSO) δ 8.68 (s, 1H), 8.04 (d, 1H), 8.01 (m, 1H), 7.58 (m, 1H), 7.47 (d, 1H), 7.33 (m, 1H), 5.18 (dd, 1H), 4.50 (m, 1H), 2.98 (d, 3H), 2.72 (m, 2H), 1.92 (m, 2H), 1.70 (m, 2H).

### EXAMPLE 284

### 1-cyclopropyl-5-fluoro-7-(4-(methylamino)-4,5,6,7-tetrahydro-1-benzothien-2-yl)-4-oxo-1,4-dihydroquinoline-3-carboxylic acid hydrochloride

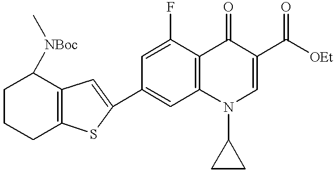

### EXAMPLE 284A

The desired product was prepared by substituting Example 285 and Example 210B for Example 266 and Example 38B, respectively, in Example 296A and was purified by silica gel chromatography eluting with 6:1 hexane/acetone to provide the desired compound as an off-white solid.

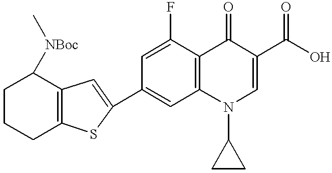

### EXAMPLE 284B

The desired product was prepared by substituting Example 284A for Example 296B in Example 296C and was used without purification.

### EXAMPLE 284C

### 1-cyclopropyl-5-fluoro-7-(4-(methylamino)-4,5,6,7-tetrahydro-1-benzothien-2-yl)-4-oxo-1,4-dihydroquinoline-3-carboxylic acid hydrochloride

The desired product was prepared as a tan solid by substituting Example 284B for Example 40B in Example 40C. MS (ESI) m/z 413 (M+H)⁺¹H NMR (300 MHz, DMSO-d₆) δ 15.05 (s, 1H), 9.40 (bs, 1H), 9.12 (bs, 1H), 8.73 (s, 1H), 8.21 (s, 1H), 8.16 (s, 1H), 7.65 (d, 1H), 4.39 (m, 1H), 3.86 (m, 1H), 2.88 (m, 2H), 2.65 (t, 3H), 2.05 (m, 3H), 1.86 (m, 1H), 1.41 (m, 2H), 1.22 (m, 2H).

### EXAMPLE 285

### ethyl 7-bromo-1-cyclopropyl-5-fluoro-4-oxo-1,4-dihydroquinoline-3-carboxylate

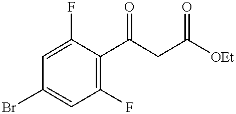

### EXAMPLE 285A

The desired product was prepared by substituting 2,6-difluoro-4-bromobenzoic acid (prepared by the method of F. Mongin and M. Schlosser Tetrahedron Letters 1996, 37, 6551-6554) for Example 269A in Example 269B and was purified by silica gel chromatography eluting with 9:1 hexane/ethyl acetate to provide the desired compound.

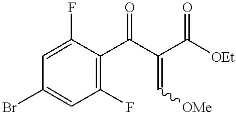

### EXAMPLE 285B

The desired product was prepared by substituting Example 285A for Example 269B in Example 269C and the crude product was used without purification.

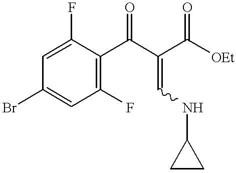

### EXAMPLE 285C

The desired product was prepared by substituting Example 285B for Example 269C in Example 269D and was used without purification.

### EXAMPLE 285D

### ethyl 7-bromo-1-cyclopropyl-5-fluoro-4-oxo-1,4-dihydroquinoline-3-carboxylate

The desired product was prepared by substituting Example 285C and acetonitrile for Example 269D and DMF, respectively, in Example 269E using reaction conditions of 95° C. for 4 hours. The crude product was purified by silica gel chromatography eluting with 9:1 hexane/acetone to provide the desired compound. MS (DCI/NH₃) m/z 356 (M+H)⁺¹H NMR (300 MHz, CDCl₃) δ 8.48 (s, 1H), 7.86 (s, 1H), 7.23 (d, 1H), 4.38 (q, 2H), 3.38 (m, 1H), 1.40 (t, 3H), 1.35 (m, 2H), 1.13 (m, 2H).

### EXAMPLE 286

### 1-(2,4-difluorophenyl)-6-fluoro-4-oxo-7-(4,5,6,7-tetrahydro-1-benzothien-2-yl)-1,4-dihydro-1,8-naphthyridine-3-carboxylic acid

### EXAMPLE 286B

The desired compound was prepared by substituting Example 286A in Example 277D for Example 2A in Example 2B and was purified by trituration with diethyl ether to yield a yellow solid. MS (DCI/NH₃) m/z 457 (M+H)⁺¹H NMR (300 MHz, DMSO) δ 9.05 (s, 1H), 8.57 (d, 1H), 7.86 (m, 1H), 7.68 (m, 1H), 7.62 (d, 1H), 2.73 (m, 2H), 2.51 (m, 2H), 1.75 (m, 4H).

### EXAMPLE 287

### 7-(4-amino-4,5,6,7-tetrahydro-1-benzothien-2-yl)-1-cyclopropyl-4-oxo-1,4-dihydroquinoline-3-carboxylic acid hydrochloride

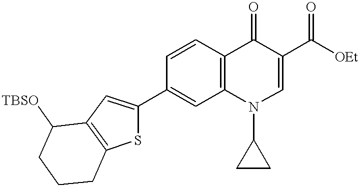

### EXAMPLE 287A

The desired product was prepared by substituting Example 38B for Example 1D and a reaction time of 8 h for 24 h in Example 1E and was purified by silica gel chromatography eluting with a gradient of 2:1 to 1:1 hexane/acetone to yield an off-white solid.

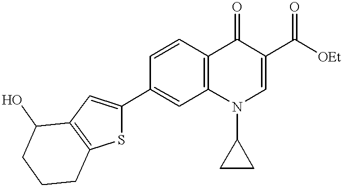

### EXAMPLE 287B

The desired product was prepared by substituting Example 287A for Example 35D in Example 35E to yield a yellow solid.

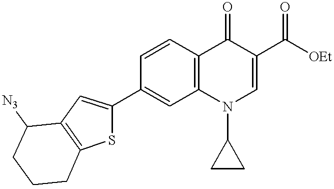

### EXAMPLE 287C

The desired product was prepared by substituting Example 287B for Example 37A in Example 37B and was purified by silica gel chromatography eluting with a gradient of 4:1 to 1:1 hexane/acetone to yield an off-white solid.

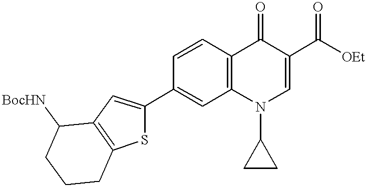

### EXAMPLE 287D

The desired product was prepared by substituting Example 287C for Example 201C in Example 201D and was purified by silica gel chromatography eluting with a gradient of 4:1 to 1:1 hexane/acetone to yield an off-white solid.

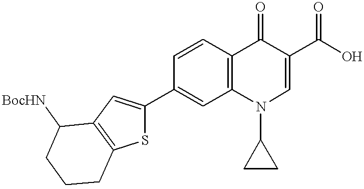

### EXAMPLE 287E

The desired product was prepared by substituting Example 287D for Example 2A and a reaction time of 2.5 h for 24 h in Example 2B to yield a yellow solid.

### EXAMPLE 287F

### 7-(4-amino-4,5,6,7-tetrahydro-1-benzothien-2-yl)-1-cyclopropyl-4-oxo-1,4-dihydroquinoline-3-carboxylic acid hydrochloride

The desired product was prepared by substituting Example 287E for Example 40B in Example 40C to yield a yellow solid. MS (DCI/NH₃) m/z 381 (M+H)⁺; ¹H NMR (300 MHz, DMSO-d6) δ 8.77 (s, 1H), 8.54 (br s, 2H), 8.42 (d, 1H), 8.35 (d, 1H), 8.08 (s, 1H), 7.83 (dd, 1H), 4.44 (m, 1H), 3.91 (m, 1H), 2.85 (m, 2H), 2.18-1.76 (m, 4H), 1.44-1.07 (m, 4H).

### EXAMPLE 288

### 7-(7-amino-4,5,6,7-tetrahydro-1-benzothien-2-yl)-1-cyclopropyl-4-oxo-1,4-dihydroquinoline-3-carboxylic acid hydrochloride

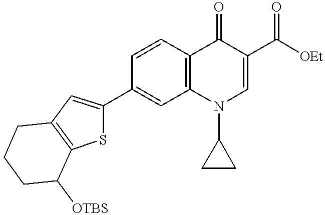

### EXAMPLE 288A

The desired product was prepared by substituting Example 41B for Example 1D and a reaction time of 8 h for 24 h in Example 1E and was purified by silica gel chromatography eluting with a gradient of 2:1 to 1:1 hexane/acetone to yield an off-white solid.

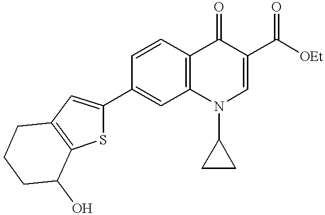

### EXAMPLE 288B

The desired product was prepared by substituting Example 288A for Example 35D in Example 35E to yield a yellow solid.

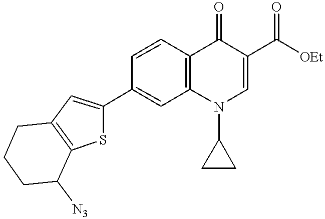

### EXAMPLE 288C

The desired product was prepared by substituting Example 288B for Example 37A in Example 37B and was purified by silica gel chromatography eluting with a gradient of 4:1 to 1:1 hexane/acetone to yield an off-white solid.

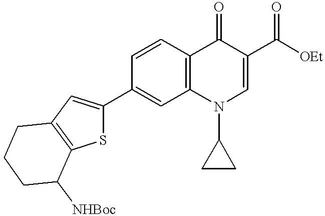

### EXAMPLE 288D

The desired product was prepared by substituting Example 288C for Example 201C in Example 201D and was purified by silica gel chromatography eluting with a gradient of 4:1 to 1:1 hexane/acetone to yield an off-white solid.

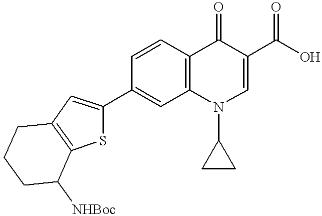

### EXAMPLE 288E

The desired product was prepared by substituting Example 288D for Example 2A and a reaction time of 2.5 h for an overnight reaction time in Example 2B to yield a yellow solid.

### EXAMPLE 288F

### 7-(7-amino-4,5 6,7-tetrahydro-1-benzothien-2-yl)-1-cyclopropyl-4-oxo-1,4-dihydroquinoline-3-carboxylic acid hydrochloride

The desired product was prepared by substituting Example 288E for Example 40B in Example 40C to yield a yellow solid. MS (DCI/NH₃) m/z 381 (M+H)⁺; ¹H NMR (300 MHz, DMSO-d₆) δ 8.77 (s, 1H), 8.58 (br s, 2H), 8.40 (d, 1H), 8.39 (s, 1H), 7.93 (dd, 1H), 7.68 (s, 1H), 4.60 (m, 1H), 3.95 (m, 1H), 2.71 (m, 2H), 2.15-1.07 (m, 8H).

### EXAMPLE 289

### 1-cyclopropyl-5,8-difluoro-7-(4-(methylamino)-4,5,6,7-tetrahydro-1-benzothien-2-yl)-4-oxo-1,4-dihydroquinoline-3-carboxylic acid hydrochloride

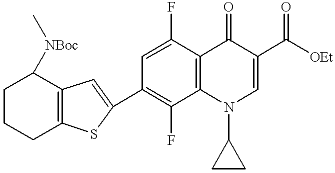

### EXAMPLE 289A

The desired product was prepared by substituting Example 323 and Example 210B for Example 266 and Example 38B, respectively, in Example 296A and was purified by silica gel chromatography eluting with 2:1 hexane/acetone to provide the desired compound as an off-white solid.

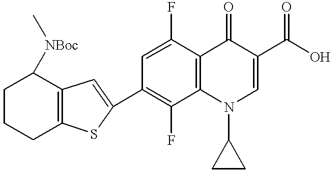

### EXAMPLE 289B

The desired product was prepared by substituting Example 289A for Example 296B in Example 296C.

### EXAMPLE 289C

### 1-cyclopropyl-5,8-difluoro-7-(4-(methylamino)-4,5,6,7-tetrahydro-1-benzothien-2-yl)-4-oxo-1,4-dihydroquinoline-3-carboxylic acid hydrochloride

The desired product was prepared as a tan solid by substituting Example 289B for Example 40B in Example 40C. MS (APCI) m/z 431 (M+H)⁺; ¹H NMR (300 MHz, DMSO-d₆) δ 9.10-8.90 (bm, 1H), 8.74 (s, 1H), 8.08 (s, 1H), 7.76 (dd, 1H), 4.41 (m, 1H), 4.21 (m, 1H), 2.90 (m, 2H), 2.66 (s, 3H), 2.10-1.81 (m, 4H), 1.18 (m, 4H).

### EXAMPLE 290

### 1-cyclopropyl-6-fluoro-7-(4-(methylamino)-4,5,6,7-tetrahydro-1-benzothien-2-yl)-4-oxo-1,4-dihydroquinoline-3-carboxylic acid hydrochloride

### EXAMPLE 290A

### ethyl 7-(4-((tert-butoxycarbonyl)(methyl)amino)-4,5,6,7-tetrahydro-1-benzothien-2-yl)-1-cyclopropyl-6-fluoro-4-oxo-1,4-dihydroquinoline-3-carboxylate

The desired product was prepared by substituting Example 210B for Example 38B in Example 296A.

### EXAMPLE 290B

### 7-(4-((tert-butoxycarbonyl)(methyl)amino)-4,5,6,7-tetrahydro-1-benzothien-2-yl)-1-cyclopropyl-6-fluoro-4-oxo-1,4-dihydroquinoline-3-carboxylic acid

The desired product was prepared by substituting Example 290A for Example 296B in Example 296C.

### EXAMPLE 290C

### 1-cyclopropyl-6-fluoro-7-(4-(methylamino)-4,5,6,7-tetrahydro-1-benzothien-2-yl)-4-oxo-1,4-dihydroquinoline-3-carboxylic acid hydrochloride

The desired product was prepared by substituting Example 290B for Example 40B in Example 40C. MS (DCI/NH₃) m/z 413 (M+H)⁺; ¹H NMR (300 MHz, DMSO-d₆) δ 9.39 (br s, 1H), 9.17 (br s, 1H), 8.78 (s, 1H), 8.51 (d, 1H), 8.21 (s, 1H), 8.16 (d, 1H), 4.45 (br m, 1H), 3.92 (m, 1H), 2.90 (m, 2H), 2.64 (s, 3H), 2.19-1.91 (m, 3H), 1.91-1.76 (m, 3H), 1.44 (m, 2H), 1.25 (m, 2H).

### EXAMPLE 291

### 1-cyclopropyl-6,8-difluoro-7-(4-(methylamino)-4,5,6,7-tetrahydro-1-benzothien-2-yl)-4-oxo-1,4-dihydroquinoline-3-carboxylic acid hydrochloride

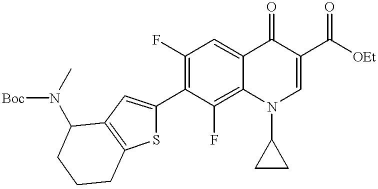

### EXAMPLE 291A

The desired product was prepared by substituting Example 324 and Example 210B for Example 266 and Example 38B, respectively, in Example 296A.

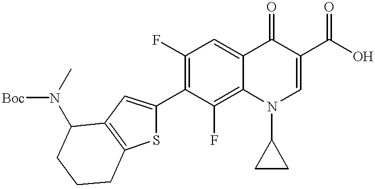

### EXAMPLE 291B

The desired product was prepared by substituting Example 291A for Example 296B in Example 296C.

### EXAMPLE 291C

### 1-cyclopropyl-6,8-difluoro-7-(4-(methylamino)-4,5,6,7-tetrahydro-1-benzothien-2-yl)-4-oxo-1,4-dihydroquinoline-3-carboxylic acid hydrochloride

The desired product was prepared by substituting Example 291B for Example 40B in Example 40C. MS (DCI/NH₃) m/z 431 (M+H)⁺; ¹H NMR (300 MHz, DMSO-d₆) δ 9.15 (br s, 2H), 8.78 (s, 1H), 8.05 (dd, 1H), 7.92 (s, 1H), 4.46 (br m, 1H), 4.20 m, 1H), 2.91 (m, 2H), 2.50 s, 3H), 2.19-1.95 (m, 3H), 1.95-1.77 (m, 1H), 1.33-1.13 (m, 4H).

### EXAMPLE 292

### 1-(2,4-difluoropheniyl)-7-(7-hydroxy-4,5,6,7-tetrahydro-1-benzothien-2-yl)-8-methoxy-4-oxo-1,4-dihydroquinoline 3-carboxylic acid

### EXAMPLE 292B

The desired compound was prepared by substituting Example 292A in Example 313A for Example 35D in Example 35E and was purified by trituration in diethyl ether to yield a yellow solid. MS (DCI/NH₃) tn/z 484 (M+H)⁺¹H NMR (300 MHz, DMSO) δ 8.60 (s, 1H), 8.32 (d, 1H), 7.77 (d, 1H), 7.60 (s, 1H), 7.49 (m, 1H), 7.07 (m, 2H), 4.92 (m, 1H), 3.12 (s, 3H), 2.64 (m, 2H), 1.80-2.17 (m, 4H).

### EXAMPLE 293

### 1-cyclopropyl-6-fluoro-7-(7-hydroxy-4,5,6,7-tetrahydro-1-benzothien-2-yl)-4-oxo-1,4-dihydroquinoline-3-carboxylic acid

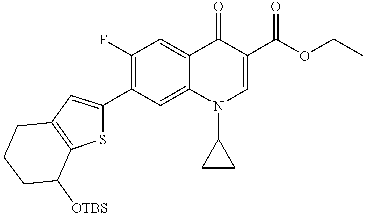

### EXAMPLE 293A

The desired product was prepared by substituting Example 41B for Example 38B in Example 296A.

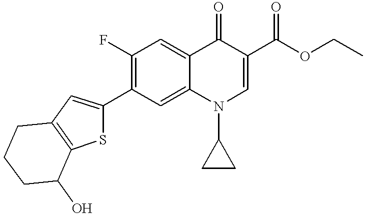

### EXAMPLE 293B

The desired product was prepared by substituting Example 293A for Example 35D in Example 35E and was used without further purification.

### EXAMPLE 293C

### 1-cyclopropyl-6-fluoro-7-(7-hydroxy-4,5,6,7-tetrahydro-1-benzothien-2-yl)-4-oxo-1,4-dihydroquinoline-3-carboxylic acid

The desired product was prepared by substituting Example 293B for Example 296B in Example 296C. MS (DCI/NH₃) m/z 400 (M+H)⁺; ¹H NMR (300 MHz, DMSO-d₆) δ 14.83 (s, 1H), 8.75 (s, 1H), 8.48 (d, 1H), 8.11 (d, 1H), 7.56 (s, 1H), 5.58 (d, 1H), 4.77 (m, 1H), 3.96 (m, 1H), 2.63 (m, 2H), 2.09-1.87 (m, 2H), 1.71-1.62 (m, 2H), 1.36 (m, 2H), 1.23 (m, 2H).

### EXAMPLE 294

### 1cyclopropyl-6,8-difluoro-7-(4-hydroxy-4,5,6,7-tetrahydro-1-benzothien-2-yl)-4-oxo-1,4-dihydroguinoline-3-carboxylic acid

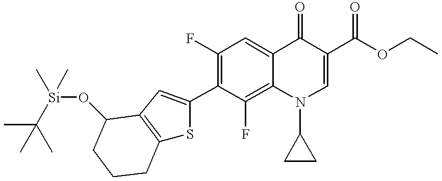

### EXAMPLE 294A

The desired product was prepared by substituting Example 324C for Example 266C in Example 296A.

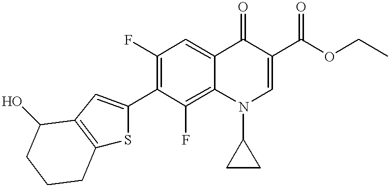

### EXAMPLE 294B

The desired product was prepared by substituting Example 294A for Example 35D in Example 35E and was used without purification.

### EXAMPLE 294C

### 1-cyclopropyl-6,8-difluoro-7-(4-hydroxy-4,5,6,7-tetrahydro-1-benzothien-2-yl)-4-oxo-1,4-dihydroguinoline-3-carboxylic acid

The desired product was prepared by substituting Example 294B for Example 296B in Example 296C. MS (DCI/NH₃) m/z 418 (M+H)⁺; ¹H NMR (300 MHz, DMSO-d₆) δ 8.76 (s, 1H), 8.02 (dd, 1H), 7.60 (s, 1H), 5.24 (d, 1H), 4.65 (m, 1H), 4.21 (m, 1H), 2.91-2.68 (m, 2H), 2.07-1.87 (m, 2H), 1.84-1.62 (m, 2H), 1.37-1.15 (m, 4H).

### EXAMPLE 295

### 1-cyclopropyl-6,8-difluoro-7-(7-hydroxy-4,5,6,7-tetrahydro-1-benzothien-2-yl)-4-oxo-1,4-dihydroquinoline-3-carboxylic acid

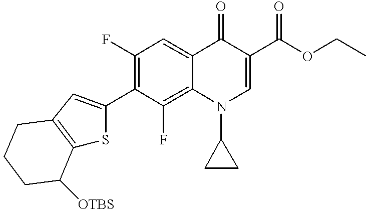

### EXAMPLE 295A

The desired product was prepared by substituting Example 324C and Example 41B for Example 266C and Example 38B, respectively, in Example 296A.

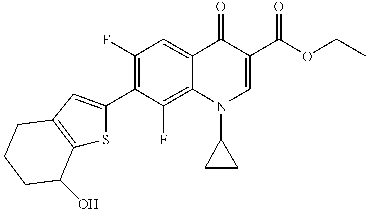

### EXAMPLE 295B

The desired product was prepared by substituting Example 295B for Example 35D in Example 35E and was used without further purification.

### EXAMPLE 295C

### 1-cyclopropyl-6,8-difluoro-7-(7-hydroxy-4,5,6,7-tetrahydro-1-benzothien-2-yl)-4-oxo-1,4-dihydroquinoline-3-carboxylic acid

The desired product was prepared by substituting Example 295B for Example 296B in Example 296C. MS (DCI/NH₃) m/z 418 (M+H)⁺; ¹H NMR (300 MHz, DMSO-d₆) δ 14.47 (s, 1H), 8.77 (s, 1H), 8.02 (dd, 1H), 7.36 (s, 1H), 5.58 (d, 1H), 4.79 (m, 1H), 4.20 (m, 1H), 2.62 (m, 2H), 2.10-1.87 (m, 2H), 1.81-1.62 (m, 2H), 1.32-1.14 (m, 4H).

### EXAMPLE 296

### 1-cyclopropyl-6-fluoro-7-(4-hydroxy-4,5,6,7-tetrahydro-1-benzothien-2-yl)-4-oxo-1,4-dihydroquinoline-3-carboxylic acid

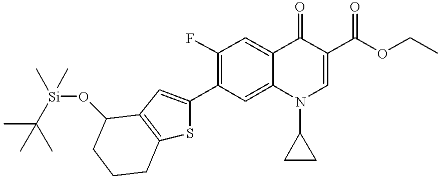

### EXAMPLE 296A

A solution of Example 266C (1.42 g, 4.00 mmol), Example 38B (12.0 ml of a 0.50 M solution in toluene, 6.00 mmol) and PdCl₂(PPh₃)₂ in toluene (25 ml) was heated to 85° C. for 5 h. The reaction mixture was allowed to cool to room temperature, was poured into 50 ml saturated aqueous KF, stirred for 1 h and filtered through Celite rinsing with ethyl acetate. The filtrate was washed with brine, dried (MgSO₄) and concentrated. The crude residue was purified by silica gel chromatography eluting with 25% then 33% acetone in hexanes to provide the desired product as a yellow solid (1.16 g, 54%).

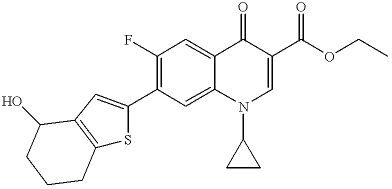

### EXAMPLE 296B

The desired product was prepared by substituting Example 296A for Example 35D in Example 35E.

### EXAMPLE 296C

### 1-cyclopropyl-6-fluoro-7-(4-hydroxy-4,5,6,7-tetrahydro-1-benzothien-2-yl)-4-oxo-1,4-dihydroquinoline-3-carboxylic acid

A solution of Example 296B (0.104 g, 0.243 mmol) in 5:1:1 THF:MeOH:H₂O (7 ml) was cooled to 0° C., treated with LiOH-H₂O (0.051 g, 1.22 mmol) and allowed to warm to ambient temperature over a 3 h period. The reaction mixture was diluted with saturated aqueous ammonium chloride, adjusted to pH5 with 8.5 % H₃PO₄ and was extracted with CH₂Cl₂ (3×20 ml). The combined organics were washed with brine, dried (MgSO₄) and concentrated. The crude solid was triturated in boiling 5:1 hexanes:acetone, filtered and dried to give the desired product (0.070 g, 72%) as a tan solid. MS (DCI/NH₃) m/z 400 (M+H)⁺; ¹H NMR (300MHz, DMSO-d₆) δ 8.76 (s, 1H), 8.46 (d,1H), 8.12 (d, 1H), 7.76 (s, 1H), 5.22 (d, 1H), 4.65 (m, 1H), 2.90-2.68 (m, 2H), 2.05-1.86 (m, 2H), 1.84-1.62 (m, 2H), 1.36 (m, 2H), 1.25 (m, 2H).

### EXAMPLE 297

### 1-(2,4-difluorophenyl)-7-(4-hydroxy-4,5,6,7-tetrahydro-1-benzothien-2-yl)-8-methoxy-4-oxo-1,4-dihydroquinoline-3-carboxylic acid

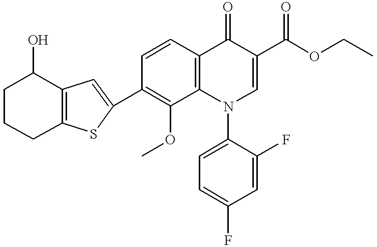

### EXAMPLE 297A

The desired compound was prepared by substituting Example 318A for Example 35D in Example 35E and was used without further purification.

### EXAMPLE 297B

### 1-(2,4-difluorophenyl)-7-(4-hydroxy-4,5,6,7-tetrahydro-1-benzothien-2-yl)-8-methoxy-4-oxo-1,4-dihydroquinoline-3-carboxylic acid

The desired compound was prepared by substituting Example 297A for Example 2A in Example 2B to yield a yellow solid that was purified by trituration in 1:1 hexane:diethyl ether. MS (DCI/NH₃) m/z 484 (M+H)⁺¹H NMR (300 MHz, DMSO-d₆) δ 8.65 (s, 1H), 8.23 (dd, 1H), 8.07 (m, 1H), 7.99 (dd, 1H), 7.63 (s, 1H), 7.56 (m, 1H), 7.35 (m, 1H), 5.14 (dd, 1H), 4.60 (m, 1H), 3.05 (s, 3H), 2.71 (m, 2H), 1.92 (m, 2H), 1.69 (m, 2H).

### EXAMPLE 298

### 1-(2,4-difluorophenyl)-6-fluoro-7-(7-hydroxy-4,5,6,7-tetrahydro-1-benzothien-2-yl)-8-methoxy-4-oxo-1,4-dihydroquinoline-3-carboxylic acid

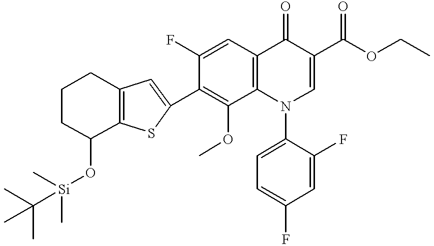

### EXAMPLE 298A

The desired product was prepared by substituting Example 276D and Example 41B for ethyl 7-bromo-1-cyclopropyl-4-oxo-1,4-dihydro-3-quinolone-3-carboxylate and Example 1D, respectively, in Example IE and was purified by silica gel chromatography eluting with 2% methanol, 0.5% ammonium hydroxide/dichloromethane to yield a dark yellow solid.

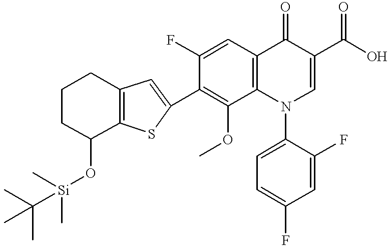

### EXAMPLE 298B

The desired compound was prepared by substituting Example 298A for Example 2A in Example 2B to yield a yellow solid that was used without further purification.

### EXAMPLE 298C

### 1(2,4-difluorophenyl)-6-fluoro-7-(7-hydroxy-4,5,6,7-tetrahydro-1-benzothien-2-yl -8-methoxy-4-oxo-1,4-dihydroquinoline-3-carboxylic acid

The desired compound was prepared by substituting Example 298B for Example 35D in Example 35E and was purified by trituration with diethyl ether to yield a yellow solid. MS (DCI/NH₃) m/z 502 (M+H)⁺¹H NMR (300 MHz, DMSO-d₆) δ 8.68 (s, 1H), 8.01 (m, 2H), 7.56 (m, 1H), 7.32 (m, 1H), 7.21 (m, 1H), 5.50 (dd, 1H), 4.73 (m, 1H), 2.96 (s, 3H), 2.57 (m, 2H), 1.95 (m, 2H), 1.68 (m, 2H).

### EXAMPLE 299

### 1-cyclopropyl-6-fluoro-7-(7-hydroxy-4,5,6,7-tetrahydro-1-benzothien-2-yl)-8-methoxy-4-oxo-1,4-dihydroquinoline-3-carboxylic acid

The desired product was prepared by substituting Example 228B for Example 2A in Example 2B and was purified by trituration in 1:1 hexane: diethyl ether to yield a yellow solid. MS (DCI/NH₃) m/z 430 (M+H)⁺¹H NMR (300 MHz, DMSO-d₆) δ 8.80 (s, 1H), 7.92 (d, 1H), 7.32 (d, 1H), 5.53 (d, 1H), 4.78 (m, 1H), 3.63 (s, 3H), 2.62 (m, 2H), 2.00 (m, 2H), 1.73 (m, 2H), 1.17 (m, 2H), 1.10 (m, 2H).

### EXAMPLE 300

### 1-cyclopropyl-5,8-difluoro-7-(4-hydroxy-4,5,6,7-tetrahydro-1-benzothien-2-yl)-4-oxo-1,4-dihydroquinoline-3-carboxylic acid

The desired product was prepared by substituting Example 325B for Example 296B in Example 296C. The reaction mixture was adjusted to pH 5 with 1M H₃PO₄ giving a yellow solid which was collected by filtration and dried. MS (DCI) m/z 418 (M+H)⁺; ¹H NMR (300 MHz, DMSO-d₆) δ 8.71 (s, 1H), 7.81 (dd, 1H), 7.80 (s, 1H), 5.18 (d, 1H), 4.62 (m, 1H), 4.20 (m, 1H), 2.81-2.70 (m, 2H), 2.02-1.62 (m, 4H), 1.18 (m, 4H).

### EXAMPLE 301

### 1-cyclopropyl-5,8-difluoro-7-(7-hydroxy-4,5,6,7-tetrahydro-1-benzothien-2-yl)-4-oxo-1,4-dihydroquinoline-3-carboxylic acid

The desired product was prepared as a yellow powder by substituting Example 326B for Example 296B in Example 296C. MS (DCI) m/z 418 (M+H)⁺; ¹H NMR (300 MHz, DMSO-d6) δ 8.71 (s, 1H), 7.83 (dd, 1H), 7.63 (s, 1H), 5.58 (d, 1H), 4.76 (m, 1H), 4.20 (m, 1H), 2.61 (m, 2H), 2.02-1.90 (m, 2H), 1.72-1.66 (m, 2H), 1.18 (m, 4H).

### EXAMPLE 302

### 1-cyclopropyl-7-(7-((4-fluorobenzyl)amino)-4,5,6,7-tetrahydro-1-benzothien-2-yl)-8-methoxy-4-oxo-1,4-dihydroquinoline-3-carboxylic acid methanesulfonic acid salt

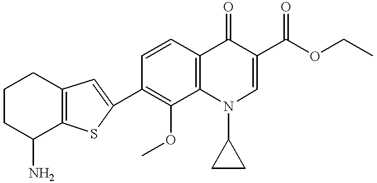

### EXAMPLE 302A

The desired product was prepared by substituting Example 228D for Example 40B in Example 40C and the crude reaction mixture partitioned between water and CH₂Cl₂. The aqueous layer was adjusted to pH 9 with NaOH, extracted with CH₂Cl₂ and the organic phase dried (Na₂SO₄) and concentrated to yield a yellow solid that was used without further purification.

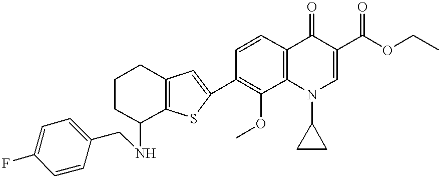

### EXAMPLE 302B

The desired product was prepared by substituting Example 302A and p-fluorobenzaldehyde for Example 83A and 3-pyridine carboxaldehyde, respectively, in Example 94A.

### EXAMPLE 302C

### 1-cyclopropyl-7-(7-((4-fluorobenzyl)amino)-4,5,6,7-tetrahydro-1-benzothien-2-yl)-8-methoxy-4-oxo-1,4-dihydroquinoline-3-carboxylic acid, methanesulfonic acid salt

The desired product was prepared by substituting Example 302B for Example 2A in Example 2B. The resulting compound was suspended in water (10 ml), treated with methanesulfonic acid (1 mol equivalent), stirred at room temperature 1 h, filtered and the filtrate freeze dried to yield the methanesulfonic acid salt as a yellow solid. MS(ESI) m/z 519 (M+H)⁺; ¹H NMR (300 MHz,DMSO-d₆) 9.25 (br s, 2H), 8.82 (s, 1H), 8.15 (d, 1H), 8.05 (d, 1H), 7.60(m, 2H), 7.31 (m, 2), 4.71 (m, 1H), 4.30 (m, 4H), 3.71 (s, 3H), 2.74 (m, 2H), 2.30 (s, 3H), 2.20 (m, 2H), 2.00 (m, 1H), 1.85 (m, 1H), 1.14 (m, 2H), 1.06 (m, 1H).

### EXAMPLE 303

### 1-cyclopropyl-5-fluoro-7-(4-hydroxy-4,5,6,7-tetrahydro-1-benzothien-2-yl)-4-oxo-1,4-dihydroquinoline-3-carboxylic acid

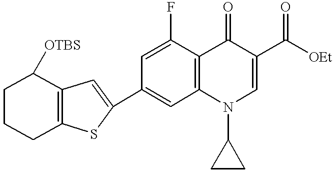

### EXAMPLE 303A

The desired product was prepared by substituting Example 285D for Example 266C in Example 296A and was purified by silica gel chromatography eluting with 2:1 hexane/acetone to provide the desired compound as an off-white solid.

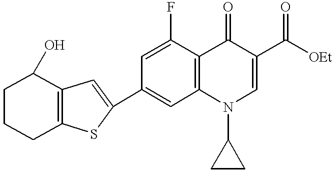

### EXAMPLE 303B

The desired product was prepared by substituting Example 303A for Example 35D in Example 35E and was used without purification.

### EXAMPLE 303C

### 1-cyclopropyl-5-fluoro-7-(4-hydroxy-4,5,6,7-tetrahydro-1-benzothien-2-yl)-4-oxo-1,4-dihydroquinoline-3-carboxylic acid

The desired product was prepared as a yellow powder by substituting Example 303B for Example 296B in Example 296C. MS (ESI) m/z 400 (M+H)⁺; ¹H NMR (300 MHz, DMSO-d₆) δ 15.05 (s, 1H), 8.70 (s, 1H), 8.10 (s, 1H), 7.78 (s, 1H), 7.69 (d, 1H), 5.17 (d, 1H), 4.60 (m, 1H), 3.89 (m, 1H), 2.76 (m, 2H), 1.94 (m, 2H), 1.73 (m, 2H), 1.34 (m, 2H), 1.21 (m, 2H).

### EXAMPLE 304

### 1-(2,4-difluorophenyl)-8-methoxy-7-(4-(methylamino)-4,5,6,7-tetrahydro-1-benzothien-2-yl)-4-oxo-1,4-dihydroquinoline-3-carboxylic acid hydrochloride

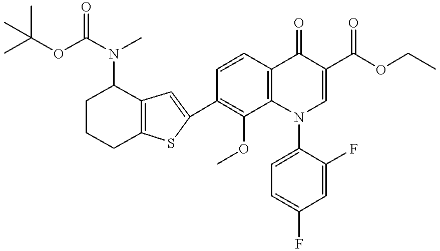

### EXAMPLE 304A

The desired product was prepared by substituting Example 327D and Example 210B for ethyl 7-bromo-1-cyclopropyl-4-oxo-1,4-dihydro-3-quinolone-3-carboxylate and Example ID, respectively, in Example 1E with a reaction time of 7h and was purified by trituration in hexane to yield a yellow solid.

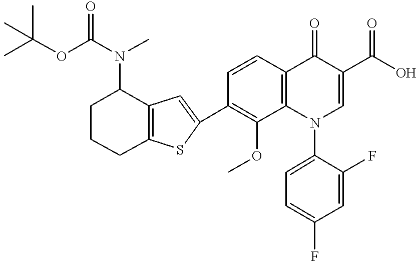

### EXAMPLE 304B

The desired compound was prepared by substituting Example 304A for Example 2A in Example 2B to yield a yellow solid that was used without flirther purification.

### EXAMPLE 304C

### 1-(2,4-difluorophenyl)-8-methoxy-7-(4-(methylamino)-4,5,6,7-tetrahydro-1-benzothien-2-yl)-4-oxo-1,4-dihydroquinoline-3-carboxylic acid hydrochloride

The desired compound was prepared by substituting Example 304B for Example 40B in Example 40C and was purified by trituration with 2:1 diethyl ether:ethanol to yield a yellow solid. MS (DCI/NH₃) m/z 497 (M+H)⁺¹H NMR (300 MHz, DMSO) δ 8.67 (s, 1H), 8.29 (d, 1H), 8.08 (m, 1H), 7.95 (dd, 1H), 7.91 (d, 1H), 7.57 (m, 1H), 7.36 (m, 1H), 4.38 (m, 1H), 3.07 (d, 3H), 2.82 (m, 2H), 2.62 (m, 3H), 2.02 (m, 3H), 1.82 (m, 1H).

### EXAMPLE 305

### 7-(4-amino-4,5,6,7-tetrahydro-1-benzothien-2-yl)-1-(2,4-difluorophenyl)-6-fluoro-8-methoxy-4-oxo-1,4-dihydroquinoline-3-carboxylic acid hydrochloride

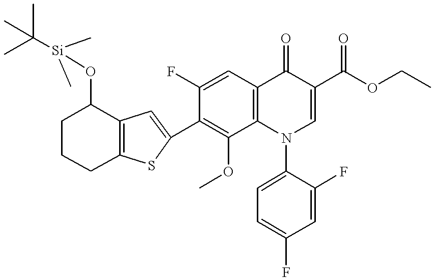

### EXAMPLE 305A

The desired product was prepared by substituting Example 276D and Example 38B for ethyl 7-bromo-1-cyclopropyl-4-oxo-1,4-dihydro-3-quinolone-3-carboxylate and Example 1D, respectively, in Example 1E with a reaction time of 5h and was purified by silica gel chromatography eluting with 2% methanol, 0.5% ammonium hydroxide/dichloromethane to yield a dark yellow solid.

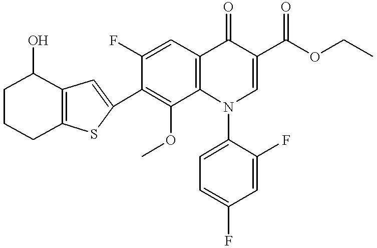

### EXAMPLE 305B

The desired compound was prepared by substituting Example 305A for Example 35D in Example 35E and was used without purification.

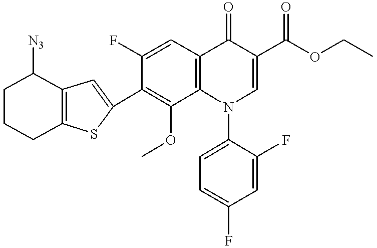

### EXAMPLE 305C

The desired compound was prepared by substituting Example 305B for Example 37A in Example 37B and was purified by silica gel chromatography eluting with 2% methanol/dichloromethane to yield a yellow solid.

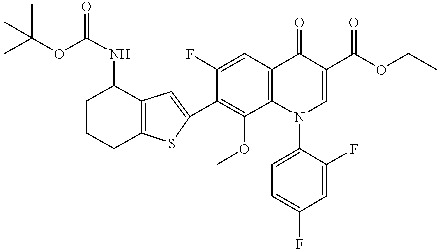

### EXAMPLE 305D

The desired compound was prepared by substituting Example 305C for Example 201C in Example 201D and was purified with silica gel chromatography eluting with 2% methanol/dichloromethane to yield a yellow solid.

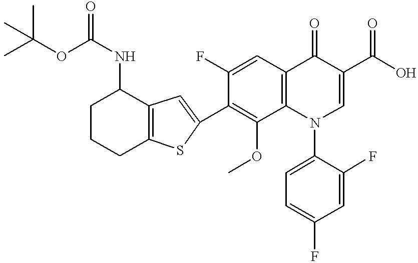

### EXAMPLE 305E

The desired compound was prepared by substituting Example 305D for Example 2A in Example 2B to yield a yellow solid that was used without purification.

### EXAMPLE 305F

### 7-(4-amino-4,5,6,7-tetrahydro-1-benzothien-2-yl)-1-(2,4-difluorophenyl)-6-fluoro-8-methoxy-4-oxo-1,4-dihydroquinoline-3 -carboxylic acid hydrochloride

The desired compound was prepared by substituting Example 305E for Example 40B in Example 40C and was purified by trituration with diethyl ether to yield a yellow solid. MS (DCI/NH₃) m/z 501 (M+H)⁺¹H NMR (300 MHz, DMSO) δ 8.70 (s, 1H), 8.28 (m, 2H), 8.08 (d, 1H), 8.03 (m, 1H), 7.70 (m, 1H), 7.59 (m, 1H), 7.35 (m, 1H), 4.43 (m, 1H), 3.01 (d, 3H), 2.83 (m, 2H), 2.05 (m, 2H), 1.84 (m, 2H).

### EXAMPLE 306

### 1-(2,4-difluorophenyl)-7-(4-(methylamino)-4,5,6,7-tetrahydro-1-benzothien-2-yl)-4-oxo-1,4-dihydro-1,8-naphthyridine-3-carboxylic acid hydrochloride

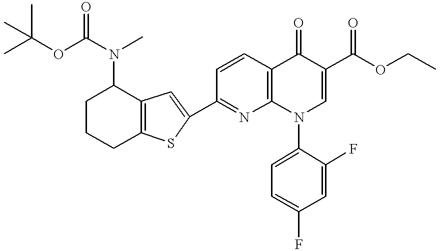

### EXAMPLE 306A

The desired product was prepared by substituting 7-chloro-1-(2,4-difluoro-phenyl)-4-oxo-1,4-dihydro-[1,8]naphthyridine-3-carboxylic acid ethyl ester (prepared by the method of Bartel, Krebs, et al DE4301246) and Example 210B for Ethyl 7-bromo-1-cyclopropyl-4-oxo-1,4-dihydro-3-quinolone-3-carboxylate and Example 1D, respectively, in Example 1E with a reaction time of 8h and was purified by trituraton in hexane to yield a light brown solid.

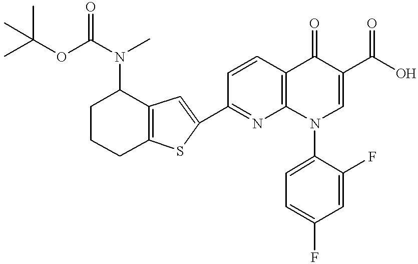

### EXAMPLE 306B

The desired compound was prepared by substituting Example 306A for Example 2A in Example 2B to yield a yellow solid that was used without purification.

### EXAMPLE 306C

### 1-(2,4-difluorophenyl)-7-(4-(methylamino)-4,5,6,7-tetrahydro-1-benzothien-2-yl)-4-oxo-1,4-dihydro-1,8-naphthyridine-3-carboxylic acid hydrochloride

The desired compound was prepared by substituting Example 306B for Example 40B in Example 40C and was purified by trituration with diethyl ether to yield a light brown solid. MS (DCI/NH₃) m/z 468 (M+H)⁺¹H NMR (300 MHz, DMSO) δ 9.25 (m, 1H), 9.04 (s, iH), 8.82 (d, 1H), 8.26 (s, 1H), 8.05 (d, 1H), 7.88 (m, iH), 7.65 (m, 1H), 7.40 (m, 1H), 4.35 (m, 1H), 2.80 (m, 2H), 2.62 (m, 3H), 2.00 (m, 3H), 1.79 (m, 1H).

### EXAMPLE 307

### 7-(4-amino-4,5,6,7-tetrahydro-1-benzothien-2-yl)-1-cyclopropyl-6-fluoro-4-oxo-1,4-dihydroquinoline-3-carboxylic acid hydrochloride

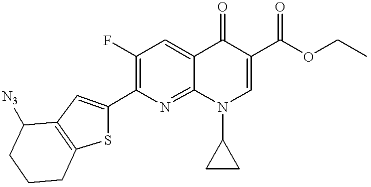

### EXAMPLE 307A

The desired product was prepared by substituting Example 296B for Example 37A in Example 38B and was purified by silica gel chromatography eluting with 30%, then 50% acetone in hexanes.

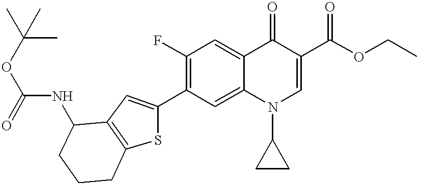

### EXAMPLE 307B

The desired product was prepared by substituting Example 307A for Example 201C in Example 201D and was purified by trituration in 25 % acetone in hexanes.

### EXAMPLE 307C

### 7-(4-amino-4,5,6,7-tetrahydro-1-benzothien-2-yl)-1-cyclopropyl-6-fluoro-4-oxo-1,4-dihydroquinoline-3-carboxylic acid hydrochloride

A solution of Example 307B (0.368 g, 0.699 mmol) in 1:1 10 % aqueous HCl:THF (15 ml) was heated to 80° C. for 10 h, allowed to cool to ambient temperature and was filtered. The solid was collected and was treated with 4M HCI in dioxane (5 ml), stirred 3 h, diluted with ethyl ether (20 ml), stirred 12 h and filtered to give the desired product as a pale yellow solid (0.259 g, 85%). MS (DCI/NH₃) m/z 399 (M+H)⁺; ¹H NMR (300 MHz, DMSO-d₆) δ 8.77 (s, 1H), 8.55 (br s, 3H), 8.49 d, 1H), 8.17 (s, 1H), 8.15 (d, 1H), 4.46 (br m, 1H), 3.92 (m, 1H), 2.87 (m, 2H), 2.22-1.97 (m, 2H), 1.93-1.77 (m, 2H), 1.43 (m, 2H), 1.24 (m, 2H).

### EXAMPLE 308

### 7-(4-amino-4,5,6,7-tetrahydro-1-benzothien-2-yl)-1-cyclopropyl-6,8-difluoro-4-oxo-1,4-dihydroquinoline-3-carboxylic acid hydrochloride

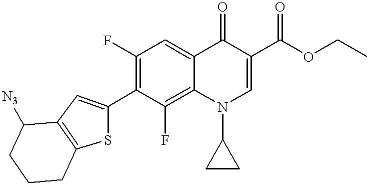

### EXAMPLE 308A

The desired product was prepared by substituting Example 294B for Example 37A in Example 37B and was purified by silica gel chromatography eluting with 30% then 50% acetone in hexanes.

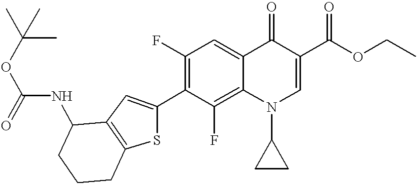

### EXAMPLE 308B

The desired product was prepared by substituting Example 308A for Example 201C in Example 201D and was purified by silica gel chromatography eluting with 1% then 2% methanol in CH₂Cl₂.

### EXAMPLE 308C

### 7-(4-amino-4,5,6,7-tetrahydro-1-benzothien-2-yl)-1-cyclopropyl-6,8-difluoro-4-oxo-1,4-dihydroquinoline-3-carboxylic acid hydrochloride

The desired product was prepared by substituting Example 308B for Example 48C in Example 48D. MS (DCI/NH₃) m/z 417 (M+H)⁺; ¹H NMR (300 MHz, DMSO-d₆) δ 8.78 (s, 1H), 8.47 (br s, 3H), 8.05 (dd, 1H), 7.87 (s, 1H), 4.46 (br m, 1H), 4.20 (m, 1H), 2.89 (m, 2H), 2.21-1.95 (m, 2H), 1.95-1.73 (m, 2H), 1.33-1.14 (m, 4H).

### EXAMPLE 309

### 7-(7-amino-4,5,6,7-tetrahydro-1-benzothien-2-yl)-1-cyclopropyl-6,8-difluoro-4-oxo-1,4-dihydroquinoline-3-carboxylic acid hydrochloride

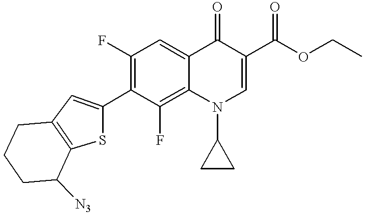

### EXAMPLE 309A

The desired product was prepared by substituting Example 295B for Example 37A in Example 37B and was purified by silica gel chromatography eluting with 30% then 50% acetone in hexanes.

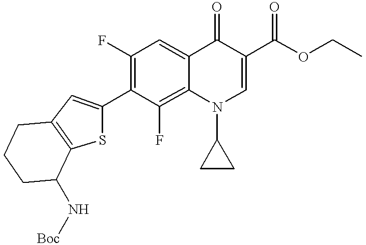

### EXAMPLE 309B

The desired product was prepared by substituting Example 309A for Example 201C in Example 201D and was purified by silica gel chromatography eluting with 1% then 2% methanol in CH₂Cl₂.

### EXAMPLE 309C

### 7-(7-amino-4,5,6,7-tetrahydro-1-benzothien-2-yl)-1-cyclopropyl-6,8-difluoro-4-oxo-1,4-dihydroquinoline-3-carboxylic acid hydrochloride

The desired product was prepared by substituting Example 309B for Example 307B in Example 307C. MS (DCI/NH₃) m/z 417 (M+H)⁺; ¹H NMR (300 MHz, DMSO-d₆) δ 8.79 (s, 1H), 8.53 (br s, 3H), 8.05 (dd, 1H), 7.45 (s, 1H), 4.65 (m, 1H), 4.20 (m, 1H), 2.87-2.60 (m, 2H), 2.23-2.08 (m, 1H), 2.07-1.90 (m, 2H), 1.90- 1.73 (m, 1H), 1.27 (m, 2H), 1.19 (m, 2H).

### EXAMPLE 310

### 7-(7-amino-4,5,6,7-tetrahydro-1-benzothien-2-yl)-1-cyclopropyl-6-fluoro-4-oxo-1,4-dihydroquinoline-3-carboxylic acid hydrochloride

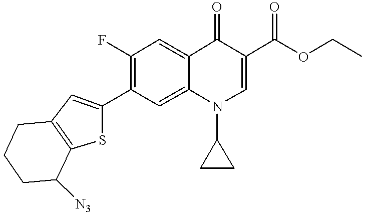

### EXAMPLE 310A

The desired product was prepared by substituting Example 293B for Example 37A in Example 37B and was purified by silica gel chromatography eluting with 30% then 50% acetone in hexanes.

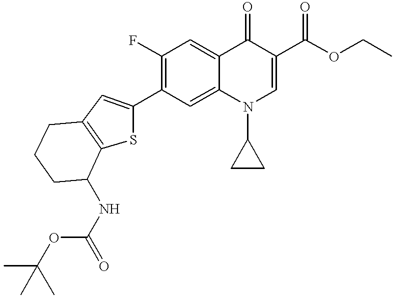

### EXAMPLE 310B

The desired product was prepared by substituting Example 31 OA for Example 201C in Example 201D and was purified by trituration in 25% acetone in hexanes.

### EXAMPLE 310C

### 7-(7-amino-4,5,6,7-tetrahydro-1-benzothien-2-yl)-1-cyclopropyl-6-fluoro-4-oxo-1,4-dihydroguinoline-3-carboxylic acid hydrochloride

The desired product was prepared by substituting Example 3 1OB for Example 307B in Example 307C. MS (DCI/NH₃) m/z 399 (M+H)⁺; ¹H NMR (300 MHz, DMSO-d₆) δ 8.78 (s, 1H), 8.56 (br s, 3H), 8.54 (d, 1H), 8.16 (d, 1H), 7.72 (s, 1H), 4.63 (m, 1H), 3.98 (m, 1H), 2.84-2.62 (m, 2H), 2.21-2.08 (m, 1H), 2.08-1.90 (m, 2H), 1.90-1.72 (m, 1H), 1.36 (m, 2H), 1.24 (m, 2H).

### EXAMPLE 311

### 1-cyclopropyl-7-(7-hydroxy-4,5,6,7-tetrahydro-1-benzothien-2-yl)-6-methyl-4-oxo-1,4-dihydroquinoline-3-carboxylic acid

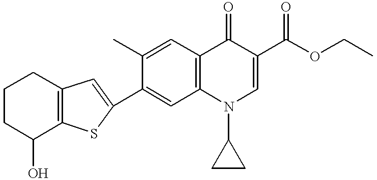

### EXAMPLE 311A

The desired compound was prepared by substituting Example 328A for Example 35D in Example 35E and was used without purification.

### EXAMPLE 311B

### 1-cyclopropyl-7-(7-hydroxy-4,5,6,7-tetrahydro-1-benzothien-2-yl)-6-methyl-4-oxo-1,4-dihydroquinoline-3-carboxylic acid

The desired compound was prepared by substituting Example 31 1A for Example 2A in Example 2B and was purified by trituration in diethyl ether to yield a yellow solid. MS (DCI/NH₃) m/z 396 (M+H)⁺¹H NMR (300 MHz, CDCl₃) δ 8.85 (s, 1H), 8.38 (s, 2H), 8.08 (s, 1H), 6.93 (s, 1H), 5.00 (m, 1H), 3.58 (m, 1H), 2.70 (m, 2H), 2.62 (s, 3H), 1.85-2.22 (m, 4H), 1.41 (m, 2H), 1.20 (m, 2H).

### EXAMPLE 312

### 1-cyclopropyl-6-methyl-7-(4-(methylamino)-4,5,6,7-tetrahydro-1-benzothien-2-yl)-4-oxo-1,4-dihydroquinoline-3-carboxylic acid hydrochloride

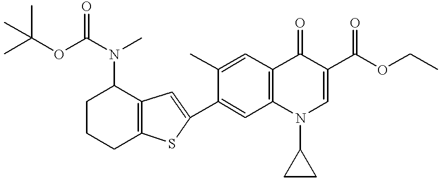

### EXAMPLE 312A

The desired product was prepared by substituting Example 269E and Example 210B for ethyl 7-bromo-1-cyclopropyl-4-oxo-1,4-dihydro-3-quinolone-3-carboxylate and Example 1D, respectively, in Example 1E and was purified by trituraton in hexane to yield a light brown solid.

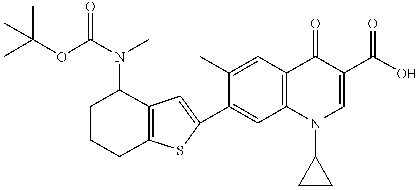

### EXAMPLE 312B

The desired compound was prepared by substituting Example 312A for Example 2A in Example 2B to yield a yellow solid that was used without purification.

### EXAMPLE 312C

### 1-cyclopropyl-6-methyl-7-(4-(methylamino)-4,5,6,7-tetrahydro-1-benzothien-2-yl)-4-oxo-1,4-dihydroquinoline-3-carboxylic acid hydrochloride

The desired compound was prepared by substituting Example 312B for Example 40B in Example 40C and was purified by trituration with 1:1 ethanol: diethyl ether to yield a solid. MS (DCI/NH₃) m/z 409 (M+H)⁺¹H NMR (300 MHz, CDCl₃) δ 8.82 (s, 1H), 8.30 (s, 1H), 8.16 (s, 1H), 7.81 (s, 1H), 4.42 (m, 1H, 3.62 (m, 1H), 2.87 (m, 1H), 2.62 (s, 3H), 2.25 (m, 1H), 2.10 (m, 1H), 1.46 (m, 1H), 1.14 (m, 1H).

### EXAMPLE 313

### 7-(7-amino-4,5,6,7-tetrahydro-1-benzothien-2-yl)-1-(2,4-difluorophenyl)-8-methoxy-4-oxo-1,4-dihydroquinoline-3-carboxylic acid hydrochloride

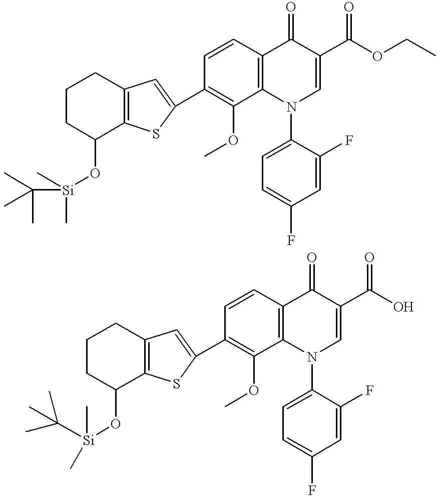

### EXAMPLE 313A EXAMPLE 292A

The desired products were prepared by substituting Example 327D and Example 41B for ethyl 7-bromo-1-cyclopropyl-4-oxo-1,4-dihydro-3-quinolone-3-carboxylate and Example 1D, respectively, in Example 1E with a reaction time of 4 h and was purified by silica gel chromatography eluting with 2% methanol, 0.5% ammonium hydroxide/dichloromethane.

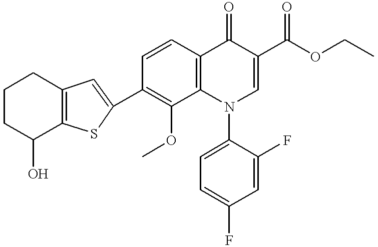

### EXAMPLE 313B

The desired compound was prepared by substituting Example 313A for Example 35D in Example 35E and was used without purification.

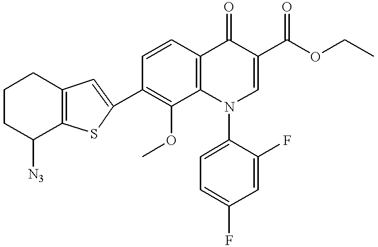

### EXAMPLE 313C

The desired compound was prepared by substituting Example 313B for Example 37A in Example 37B and was purified by silica gel chromatography eluting with 2% methanol/dichloromethane to yield a yellow solid.

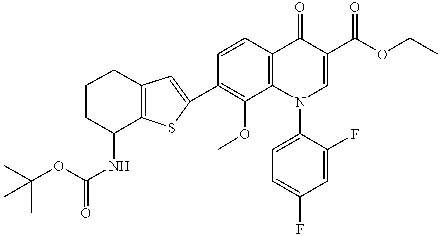

### EXAMPLE 313D

The desired compound was prepared by substituting Example 313C for Example 201C in Example 201D and was purified with silica gel chromatography eluting with 2% methanol/dichloromethane to yield a yellow solid.

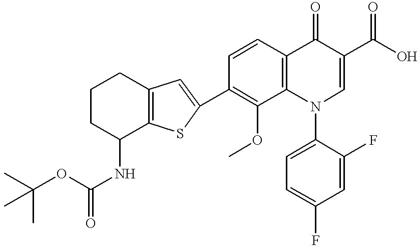

### EXAMPLE 313E

The desired compound was prepared by substituting Example 313D for Example 2A in Example 2B to yield a yellow solid that was used without purification.

### EXAMPLE 313F

### 7-(7-amino-4,5,6,7-tetrahydro-1-benzothien-2-yl)-1-(2,4-difluorophenyl)-8-methoxy-4-oxo-1,4-dihydroquinoline-3-carboxylic acid hydrochloride

The desired compound was prepared by substituting Example 313E for Example 40B in Example 40C and was purified by trituration with diethyl ether to yield a yellow solid. MS (DCI/NH₃) m/z 483 (M+H)⁺¹H NMR (300 MHz, CD₃OD) δ 8.72 (s, 1H), 8.35 (d, 1H), 7.95 (dd, 1H), 7.87 (m, 1H), 7.47 (d, 1H), 7.24 (m, 2H), 4.63 (m, 1H), 3.14 (s, 3H), 2.77 (m, 2H), 2.25 (m, 2H), 2.00 (m, 2H).

### EXAMPLE 314

### 8-chloro-1-cyclopropyl-7-(4-hydroxy-4,5 6,7-tetrahydro-1-benzothien-2-yl)-4-oxo-1,4-dihydroquinoline-3-carboxylic acid

The desired product was prepared by substituting Example 329B for Example 2A and a reaction time of 2.5 h for an overnight reaction time in Example 2B to yield a light tan solid. MS (DCI/NH₃) m/z 416 (M+H)⁺; ¹H NMR (300 MHz, CDCl₃) δ 8.99 (s, 1H), 8.39(d, 1H), 7.66 (d, 1H), 7.43 (s, 1H), 4.85 (m, 1H), 4.40 (m, 1H), 2.95-2.76 (m, 2H), 2.10-1.85 (m, 4H), 1.37-1.00 (m, 4H).

### EXAMPLE 315

### 8-chloro-1-cyclopropyl-7-(7-hydroxy-4,5,6,7-tetrahydro-1-benzothien-2-yl)-4-oxo-1,4-dihydroquinoline-3-carboxylic acid

The desired product was prepared by substituting Example 330B for Example 2A and a reaction time of 2.5 h for an overnight reaction time in Example 2B to yield a light tan solid. MS (DCI/NH₃) m/z 416 (M+H)⁺; ¹H NMR (300 MHz, DMSO-d₆) δ 14.5 (s, 1H), 8.92(s, 1H), 8.29 (d, 1H), 7.79 (d, 1H), 7.28 (s, 1H), 4.76 (m, 1H), 4.46 (m, 1H), 2.61 (m, 2H), 2.06-1.68 (m, 4H), 1.31-1.04 (m, 4H).

### EXAMPLE 316

### 1-cyclopropyl-6-fluoro-7-(4-hydroxy-4,5,6,7-tetrahydro-1-benzothien-2-yl)-8-methyl-4-oxo-1,4-dihydroquinoline-3-carboxylic acid

The desired product was prepared by substituting Example 321B for Example 317A in Example 317B. MS (DCI/NH₃) m/z 414 (M+H)⁺; ¹H NMR (DMSO-d₆, 300 MHz) δ 8.89 (s, 11H), 7.94 (d, 11H), 6.95 (s, 11H), 4.63 (m, 2H), 4.38 (m, 1H), 2.77 (s, 3H), 2.68-2.81 (br s, 2H), 2.09 (s, 3H), 1.72-1.97 (m, 4H), 1.02-1.24 (m, 4H).

### EXAMPLE 317

### 1-cyclopropyl-6-fluoro-8-methyl-7-(4-(methylamino)-4,5,6,7-tetrahydro-1-benzothien-2-yl)-4-oxo-1,4-dihydroquinoline-3-carboxylic acid hydrochloride

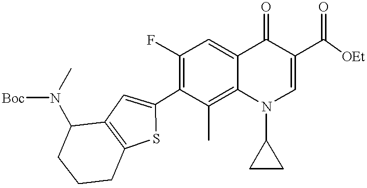

### EXAMPLE 317A

The desired product was prepared by substituting Example 21 OB and Example 331C for Example 38B and Example 266C in Example 296A, and was purified by silica gel chromatography eluting with a gradient of 0% to 20% acetone in hexanes to provide the desired product as a yellow solid.

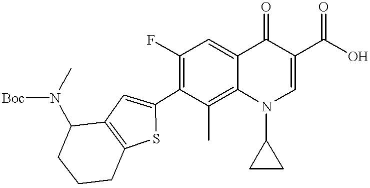

### EXAMPLE 317B

A solution of Example 317A (0.440 g, 0.794 mmol) in 20 mL of 4:1 THF-H₂O was cooled to 0° C. and treated with LiOH.H₂O (0.201 g, 4.80 mmol), followed by warming to ambient temperature for 4 h. The reaction mixture was poured into 10 mL saturated aqueous NH₄Cl, adjusted to pH 6 with 8.5% H₃PO₄, extracted with ethyl acetate, and the combined organic phases washed with brine, dried (Na₂SO₄), and concentrated to give the desired product (0.46 g, 100%). The crude residue was used without further purification.

### EXAMPLE 317C

### 1-cyclopropyl-6-fluoro-8-methyl-7-(4-(methylamino)-4,5,6,7-tetrahydro-1-benzothien-2-yl)-4-oxo-1,4-dihydroquinoline-3-carboxvlic acid hydrochloride

The desired product was prepared by substituting Example 317B for Example 40B in Example 40C to provide the desired product as a yellow solid. MS (DCI/NH₃) m/z 427 (M+H)⁺; ¹H NMR (DMSO-d₆, 300 MHz) δ 9.49 (m, 1H), 9.25 (m, 1H), 8.91 (s, 1H), 7.96 (d, 1H), 7.62 (s, 1H), 4.40 (m, 2H), 2.87 (br s, 2H), 2.78 (s, 3H), 2.59 (dd, 3H), 1.83-2.10 (m, 4H), 1.01-1.28 (m, 4H).

### EXAMPLE 318

### 7-(4-amino-4,5,6,7-tetrahydro-1-benzothien-2-yl)-1-(2,4-difluorophenyl)-8-methoxy-4-oxo-1,4-dihydroquinoline-3-carboxylic acid hydrochloride

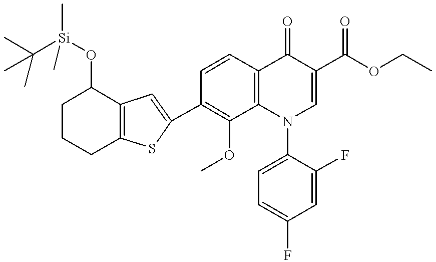

### EXAMPLE 318A

The desired product was prepared by substituting Example 327D and Example 38B for ethyl 7-bromo-1-cyclopropyl-4-oxo-1,4-dihydro-3-quinolone-3-carboxylate and Example 1D, respectively, in Example 1E with a reaction time of 4h and was purified by silica gel chromatography eluting with 2% methanol, 0.5% ammonium hydroxide/dichloromethane to yield a yellow solid.

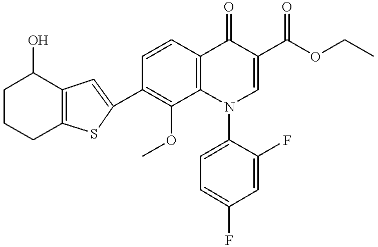

### EXAMPLE 318B

The desired compound was prepared by substituting Example 318A for Example 35D in Example 35E and was used without purification.

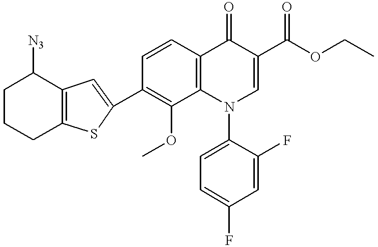

### EXAMPLE 318C

The desired compound was prepared by substituting Example 318B for Example 37A in Example 37B and was purified by silica gel chromatography eluting with 2% methanol/dichloromethane to yield a yellow solid.

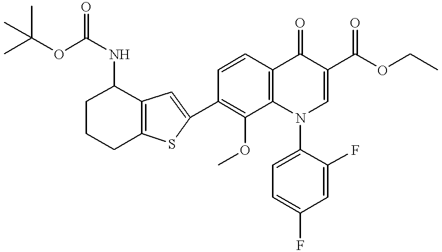

### EXAMPLE 318D

The desired compound was prepared by substituting Example 318C for Example 201C in Example 201D and was purified with silica gel chromatography eluting with 2% methanol/dichloromethane to yield a yellow solid.

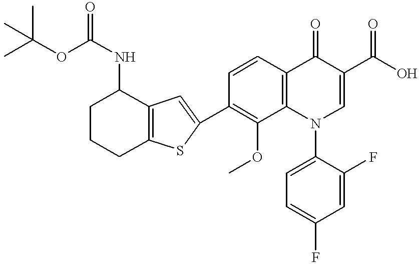

### EXAMPLE 318E

The desired compound was prepared by substituting Example 318D for Example 2A in Example 2B to yield a yellow solid that was used without purification.

### EXAMPLE 318F

### 7-(4-amino-4,5,6,7-tetrahydro-1-benzothien-2-yl)-1-(2,4-difluorophenyl)-8-methoxy-4-oxo-1,4-dihydroquinoline-3-carboxylic acid hydrochloride

The desired compound was prepared by substituting Example 318E for Example 40B in Example 40C and was purified by trituration with diethyl ether to yield a yellow solid. MS (DCI/NH₃) m/z 483 (M+H)⁺¹H NMR (300 MHz, DMSO-d₆) δ 8.68 (s, 1H), 8.30 (m, 1H), 8.08 (m, 1H), 7.88 (d, 1H), 7.83 (d, 1H), 7.58 (m, 1H), 7.35 (m, 1H), 4.40 (m, 1H), 3.07 (d, 3H), 2.80 (m, 2H), 2.03 (m, 2H), 1.82 (m, 2H).

### EXAMPLE 319

### 5-amino-1-cyclopropyl-6,8-difluoro-7-(7-hydroxy-4,5,6,7-tetrahydro-1-benzothien-2-yl)-4-oxo-1,4-dihydroquinoline-3-carboxylic acid

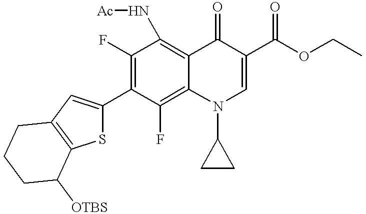

### EXAMPLE 319A

The desired product was prepared by substituting Example 332D and Example 41B for Example 266C and Example 38B, respectively in Example 296A.

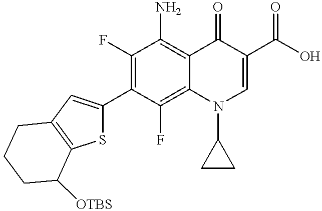

### EXAMPLE 319B

The desired product was prepared by substituting Example 319A for Example 333D in Example 333E.

### EXAMPLE 319C

### 5-amino-1-cyclopropyl-6,8-difluoro-7-(7-hydroxy-4,5,6,7-tetrahydro-1-benzothien-2-yl)-4-oxo-1,4-dihydroquinoline-3-carboxylic acid

The desired product was prepared by substituting Example 319B for Example 35D in Example 35E and was purified by trituration in 20% acetone in hexanes. MS (DCI/NH₃) m/z 433 (M+H)⁺; ¹H NMR (300 MHz, DMSO-d₆) δ 14.42 (s, 1H), 8.61 (s, 1H), 7.45 (br s, 2H), 7.28 (s, 1H), 5.54 (d, 1H), 4.77 (m, 1H), 4.10 (m, 1H), 2.62 (m, 2H), 2.10-1.87 (m, 2H), 1.78-1.63 (m, 2H), 1.13 (m, 4H).

### EXAMPLE 320

### 1-cyclopropyl-6-fluoro-7-(7-hydroxy-4,5,6,7-tetrahydro-1-benzothien-2-yl)-4-oxo-1,4-dihydro-1,8-naphthyridine-3-carboxylic acid

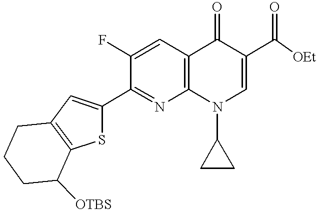

### EXAMPLE 320A

The desired product was prepared by substituting ethyl 1-cyclopropyl-7-chloro-6-fluoro-4-oxo-1,4-dihydro[1,8]naphthyridine-3-carboxylate (prepared by the method of Bouzard et al., J. Med. Chem., 1989, 32, 537-542), Example 41B, and a reaction time of 4 h for ethyl 7-bromo-1-cyclopropyl-4-oxo-1,4-dihydro-3-quinolone-3-carboxylate, Example 1D, and 24 h, respectively, in Example 1E and was purified by triturating with acetone and hexanes to yield a light gray solid.

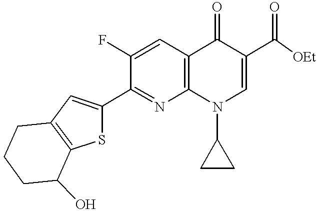

### EXAMPLE 320B

The desired product was prepared by substituting Example 320A for Example 35D in Example 35E to yield an off-white solid.

### EXAMPLE 320C

The desired product was prepared by substituting Example 320B or Example 2A and a reaction time of 2.5 h for an overnight reaction time in Example 2B to yield a yellow solid. MS (DCI/NH₃) m/z 401 (M+H)⁺; ¹H NMR (300 MHz, DMSO-d₆) δ 8.79 (s, 1H), 8.50 (d, 1H), 7.73 (d, 1H), 4.77 (m, 1H), 3.84 (m, 1H), 2.63 (m, 2H), 2.03-1.66 (m, 4H), 1.32-1.17 (m, 4H).

### EXAMPLE 321

### 7-(4-amino-4,5,6,7-tetrahydro-1-benzothien-2-yl)-1-cyclopropyl-6-fluoro-8-methyl-4-oxo-1,4-dihydroquinoline-3-carboxylic acid hydrochloride

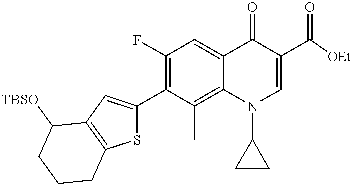

### EXAMPLE 321A

The desired product was prepared by substituting Example 33 1C for Example 266C in Example 296A.

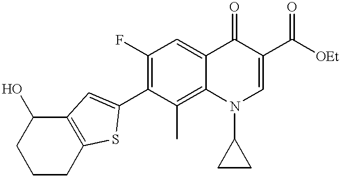

### EXAMPLE 321B

The desired product was prepared by substituting Example 321A for Example 35D in Example 35E and was purification by trituration with 5% acetone in hexanes.

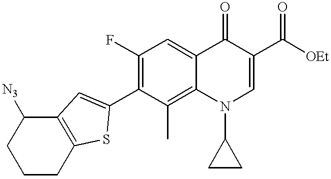

### EXAMPLE 321C

A solution of Example 321B (0.679 g, 1.54 mmol) in dichloromethane (20 mL) was cooled to 0° C., treated sequentially with DPPA (0.66 mL, 3.05 mmol) and DBU (0.46 mL, 3.08 mmol), followed by warming to ambient temperature for 16 h. The reaction mixture was washed with water, 1N HCl, brine, dried (Na₂SO₄), and concentrated. The crude residue was purified by chromatography on silica gel eluting with a gradient of 0% to 25% acetone in hexanes to provide the desired product as an off-white solid (0.528 g, 74%).

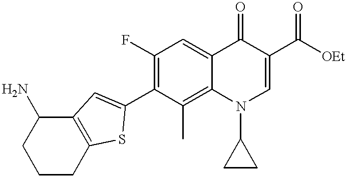

### EXAMPLE 321D

A solution of Example 321C (0.4075 g, 0.874 mmol) in 1:1 ethyl acetate-methanol (20 mL) was treated with 10% Pd/C (104 mg) and 1 atm hydrogen gas for 24 h. The solution was filtered through celite and concentrated to provide the desired product as a yellow solid (0.319 g, 83%) that was used without purification.

### EXAMPLE 321E

### 7-(4-amino-4,5,6,7-tetrahydro-1-benzothien-2-yl)-1-cyclopropyl-6-fluoro-8-methyl-4-oxo-1,4-dihydroquinoline-3-carboxylic acid hydrochloride

The desired product was prepared by substituting Example 321D for Example 48C in Example 48D. MS (DCI/NH₃) m/z 413 (M+H)⁺; ¹H NMR (DMSO-d₆, 300 MHz) 68.91 (s, 1H), 8.53 (br s, 3H), 7.97 (d, 1H), 7.49 (s, 1H), 4.42 (m, 2H), 2.87 (br s, 2H), 2.78 (s, 3H), 1.87-2.15 (m, 4H), 1.02-1.31 (m, 4H)

### EXAMPLE 322

### 1-cyclopropyl-7-(7-hydrox-4,5,6,7-tetrahydro-1-benzothien-2-yl)-6-methyl-4-oxo-1,4-dihydro-1,8-naphthyridine-3-carboxylic acid

The desired compound was prepared by substituting Example 334C for Example 2A in Example 2B and was purified by trituration with diethyl ether to give a yellow solid. MS (DCI/NH₃) m/z 397 (M+H)⁺¹H NMR (300 MHz, DMSO-d₆) δ 14.82 (s, 1H), 8.78 (s, 1H), 8.55 (2, 1H), 7.62 (s, 1H), 5.53 (d, 1H), 4.77 (m, 1H), 3.85 (m, 1H), 2.73 (s, 3H), 2.63 (m, 2H), 1.98 (m, 2H), 1.70 (m, 2H), 1.28 (m, 2H), 1.16 (m, 2H).

### EXAMPLE 323

### ethyl 7-bromo-1-cyclopropyl-5,8-difluoro-4-oxo-1,4-dihydroquinoline-3-carboxylate

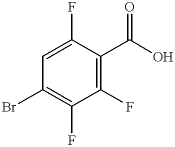

### EXAMPLE 323A

A solution of n-BuLi (50 mL of a 2.5 M solution in hexanes, 125 mmol) in THF (200 mL) was cooled to −78° C., treated dropwise with 2,2,6,6-tetramethylpiperidine, stirred for 15 minutes and treated dropwise with a solution of 1-bromo-2,3,5-trifluorobenzene (Lancaster Synthesis 25 g, 118 mmol) in 20 mL of THF. The solution was stirred for 2 hours at −78° C., treated with bubbling CO₂ (g) for 30 minutes, warmed to 0° C. and concentrated. The residue was dissolved in water, washed with diethyl ether, acidified to pH 1 with concentrated HCl and extracted 3×200 mL with dichloromethane. The extracts were combined, washed with brine, dried (MgSO₄), and concentrated to give the desired product (13 g, 43%) as a yellow solid.

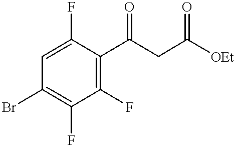

### EXAMPLE 323B

The desired product was prepared as an amber oil by substituting Example 323A for Example 269A in Example 269B and was used without purification.

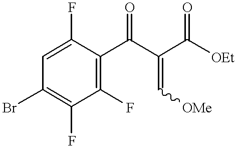

### EXAMPLE 323C

The desired product was prepared by substituting Example 323B for Example 269B in Example 269C and was used without purification.

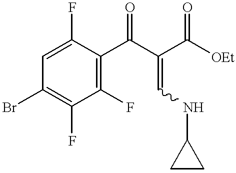

### EXAMPLE 323D

The desired product was prepared by substituting Example 323C for Example 269C in Example 269D and isolated as a white solid.

### EXAMPLE 323E

### ethyl 7-bromo-1-cyclopropyl-5,8-difluoro-4-oxo-1,4-dihydroquinoline-3-carboxylate

A solution of Example 323D (26.0 g, 66.5 mmol) in 1,4-dioxane (70 mL) at 0° C., was treated dropwise with potassium tert-butoxide (1M in THF, 70 mL, 70 mmol), stirred for 1 hour at 10° C., poured into ice water and the resulting solid was collected by filtration and dried to give the desired product as a white solid (23 g, 95%). MS (DCI) m/z 372/374 (M+H)⁺; ¹H NMR (300 MHz, DMSO-d6) δ 8.40 (s, 1H), 7.68 (dd, 1H), 4.22 (q, 2H), 3.96 (m, 1H), 1.27 (t 3H), 1.11 (d, 4H).

### EXAMPLE 324

### ethyl 7-bromo-1-cyclopropyl-6,8-difluoro-4-oxo-1,4-dihydroqiuinoline-3-carboxylate

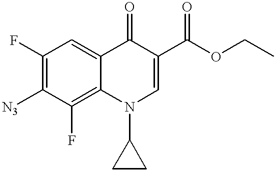

### EXAMPLE 324A

The desired product was prepared by substituting ethyl 1-cyclopropyl-6,7,8-trifluoro-4-oxo-1,4-dihydro-quinoline-3-carboxylate (Domagala, J. M., Heifetz, C. L., Hutt, M. P., Mich, T. F., Nichols, J. B. J. Med. Chem. 1988, 31, 991-1001.) for ethyl l-cyclopropyl-6,7-difluoro-4-oxo-1,4-dihydro-quinoline-3-carboxylate in Example 266.

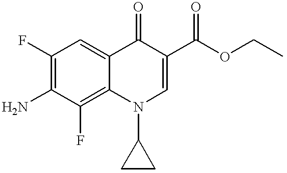

### EXAMPLE 324B

The desired product was prepared by substituting Example 324A for Example 266A in Example 266B.

### EXAMPLE 324C

### ethyl 7-bromo-1-gyclopropyl-6,8-difluoro4-oxo-1,4-dihydroquinoline-3-carboxylate

The desired product was prepared by substituting Example 324B for Example 266B in Example 266C. MS (DCI/NH₃) m/z 372 (M+H)⁺; ¹H NMR (300 MHz, DMSO-d₆) δ 8.49 (s, 1H), 7.88 (dd, 1H), 4.23 (q, 2H), 4.01 (m, 1H), 1.28 (t, 3H), 1.16 (m, 4H).

### EXAMPLE 325

### 7-(4-amino-4,5,6,7-tetrahydro-1-benzothien-2-yl)-1-cyclopropyl-5,8-difluoro-4-oxo-1,4-dihydroquinoline-3-carboxylic acid hydrochloride

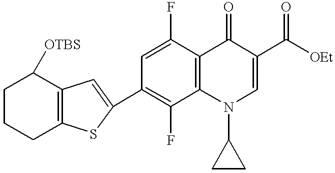

### EXAMPLE 325A

The desired product was prepared by substituting Example 323E for Example 266C, in Example 296A and was purified by silica gel chromatography eluting with 2:1 hexane/acetone to provide the desired compound.

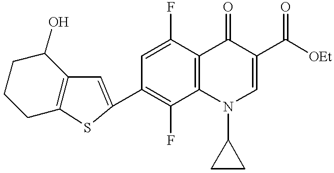

### EXAMPLE 325B

The desired product was prepared by substituting Example 325A for Example 35D in Example 35E and was purified by silica gel chromatography eluting with 2:1 hexane/acetone to provide the desired compound.

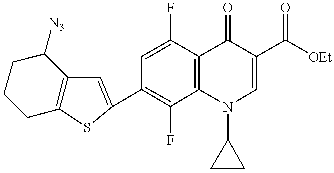

### EXAMPLE 325C

The desired product was prepared by substituting Example 325B for Example 37A in Example 37B using dichloromethane as both the reaction and extraction solvent. The crude product was purified by silica gel chromatography eluting with 2:1 hexane/acetone to provide the desired compound.

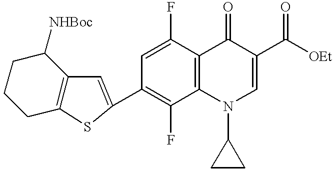

### EXAMPLE 325D

The desired product was prepared by substituting Example 325C for Example 201C in Example 201D and was purified by silica gel chromatography eluting with 1:1 hexane/acetone to provide the desired compound.

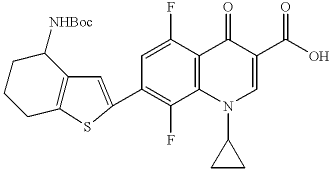

### EXAMPLE 325E

The desired product was prepared by substituting Example 325D for Example 296B in Example 296C.

### EXAMPLE 325F

### 7-(4-amino-4,5,6,7-tetrahydro-1-benzothien-2-yl)-1-cyclopropyl-5,8-difluoro-4-oxo-1,4-dihydroquinoline-3-carboxylic acid hydrochloride

The desired product was prepared as a tan solid by substituting Example 325E for Example 40B in Example 40C. MS (DCI) m/z 417 (M+H)⁺; ¹H NMR (300 MHz, DMSO-d₆) δ 8.74 (s, 1H), 8.36 (bs, 2H), 7.98 (s, 1H), 7.62 (dd, 1H), 4.45 (m, 1H), 4.20 (m, 1H), 2.88 (m, 2H), 2.18-1.80 (m, 4H), 1.21-1.18 (m, 4H).

### EXAMPLE 326

### 7-(7-amino4,5,6,7-tetrahydro-1-benzothien-2-yl)-1-cyclopropyl-5,8-difluoro-4-oxo-1,4-dihydroquinoline-3-carboxylic acid hydrochloride

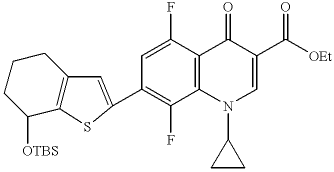

### EXAMPLE 326A

The desired product was prepared by substituting Example 323E and Example 41B for Example 266C and Example 38B, respectively, in Example 296A and was purified by silica gel chromatography eluting with 2:1 hexanelacetone to provide the desired compound.

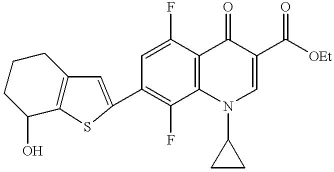

### EXAMPLE 326B

The desired product was prepared by substituting Example 326A for Example 35D in Example 35E and was purified by silica gel chromatography eluting with 2:1 hexane/acetone to provide the desired compound.

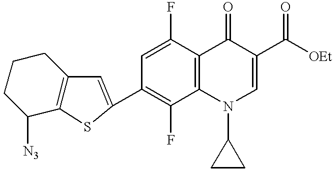

### EXAMPLE 326C

The desired product was prepared by substituting Example 326B for Example 37A in Example 37B using dichloromethane as both the reaction solvent and workup extraction solvent. The crude product was purified by silica gel chromatography eluting with 2:1 hexane/acetone to provide the desired compound.

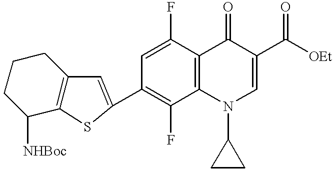

### EXAMPLE 326D

The desired product was prepared by substituting Example 326C for Example 201C in Example 201D and was purified by silica gel chromatography eluting with 1:1 hexane/acetone to provide the desired compound.

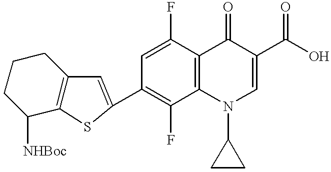

### EXAMPLE 326E

The desired product was prepared by substituting Example 326D for Example 296B in Example 296C.

### EXAMPLE 326F

### 7-(7-amino-4,5,6,7-tetrahydro-1-benzothien-2-yl)-1-cyclopropyl-5,8-difluoro-4-oxo-1,4-dihydroquinoline-3-carboxylic acid hydrochloride

The desired product was prepared as a yellow solid by substituting Example 326E for Example 40B in Example 40C. MS (DCI) m/z 417 (M+H)⁺; ¹H NMR (300 MHz, DMSO-d₆) δ 8.74 (s, 1H), 8.49 (bs, 2H), 7.90 (dd, 1H), 7.76 (s, 1H), 4.63 (m, 1H), 4.20 (m, 1H), 2.69 (m, 2H), 2.18-1.73 (m, 4H), 1.22-1.16 (m, 4H).

### EXAMPLE 327

### ethyl 7-bromo-1-(2,4-difluorophenyl)-8-methoxy-4-oxo-1,4-dihydroquinoline-3-carboxylate

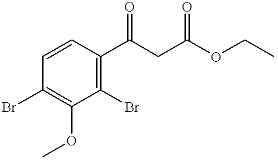

### EXAMPLE 327A

The desired product was prepared by substituting 2,4-dibromo-3-methoxy benzoic acid (prepared by the method of Guerry, Jolidon, et. al. WO9616046) for Example 269A in Example 269B and was purified by silica gel chromatography eluting with 1% methanol/dichloromethane to yield a white solid.

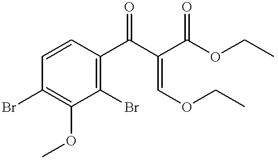

### EXAMPLE 327B

The desired product was prepared by substituting Example 327A for Example 269B in Example 269C to yield an oil that was used without purification.

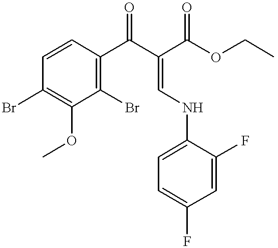

### EXAMPLE 327C

The desired product was prepared by substituting Example 327B and 2,4-difluoroaniline for Example 269C and cyclopropylamine, respectively, in Example 269D to yield an oil that was used without purification.

### EXAMPLE 327D

### ethyl 7-bromo-1-(2,4-difluorophenyl)-8-methoxy-4-oxo-1,4-dihydroquinoline-3-carboxylate

The desired product was prepared by substituting Example 327C for Example 269D in Example 269E and was purified by trituration with diethyl ether to yield a white solid. MS (DCI/NH₃) m/z 440 (M+H)⁺¹H NMR (300 MHz, CDCl₃) δ 8.27 (s, 1H), 8.21 (d, 1H), 7.62 (d, 1H), 7.48 (m, 1H), 7.05 (m, 2H), 4.39 (q, 2H), 3.29 (s, 3H), 1.39 (t, 3H).

### EXAMPLE 328

### 7-(7-amino-4,5,6,7-tetrahydro-1-benzothien-2-yl)-1-cyclopropyl-6-methyl-4-oxo-1,4-dihydroquinoline-3-carboxylic acid hydrochloride

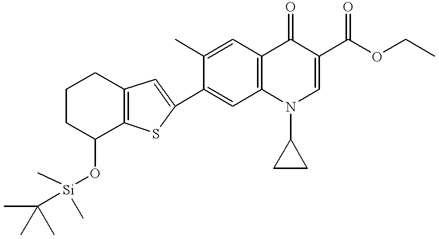

### EXAMPLE 328A

The desired product was prepared by substituting Example 269E and Example 41B for ethyl 7-bromo-1-cyclopropyl-4-oxo-1,4-dihydro-3-quinolone-3-carboxylate and Example 1D, respectively, in Example IE with a reaction time of 2h and was purified by silica gel chromatography eluting with 2% methanol and 0.5% ammonium.

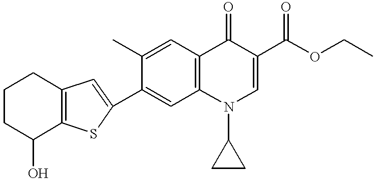

### EXAMPLE 328B

The desired compound was prepared by substituting Example 328A for Example 35D in Example 35E and was used without purification.

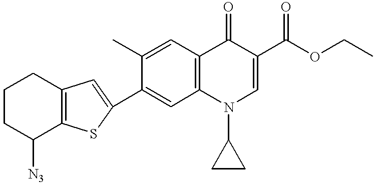

### EXAMPLE 328C

The desired compound was prepared by substituting Example 328B for Example 37A in Example 37B and was purified by silica gel chromatography eluting with 2% methanol/dichloromethane to yield a yellow solid.

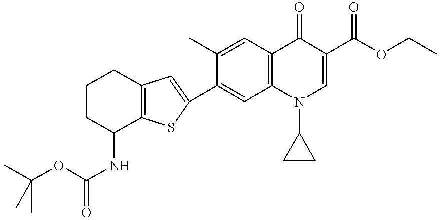

### EXAMPLE 328D

The desired compound was prepared by substituting Example 328C for Example 201C in Example 201D and was purified with silica gel chromatography eluting with 2% methanol/dichloromethane to yield a yellow solid.

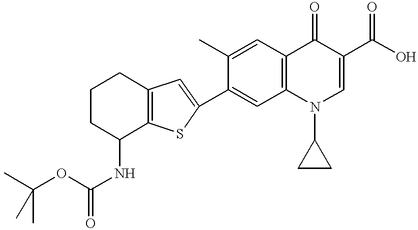

### EXAMPLE 328E

The desired compound was prepared by substituting Example 328D for Example 2A in Example 2B to yield a yellow solid that was used without purification.

### EXAMPLE 328F

### 7-(7-amino-4,5,6,7-tetrahydro-1-benzothien-2-yl)-1-cyclopropyl-6-methyl-4-oxo-1,4-dihydroquinoline-3-carboxylic acid hydrochloride

The desired compound was prepared by substituting Example 328E for Example 40B in Example 40C and was purified by trituration with diethyl ether to yield a solid. MS (DCI/NH₃) m/z 395 (M+H)⁺; ¹H NMR (300 MHz, DMSO-d₆) δ 8.91 (s, 1H), 8.36 (s, 1H0, 8.25 (s, 1H), 7.16 (s, 1H), 3.81 (m, 1H), 3.66 (m, 1H), 2.82 (m, 2H), 2.61 (s, 3H), 2.32 (m, 2H), 2.04 (m, 2H), 1.38 (m, 2H0, 1.25 (m, 2H).

### EXAMPLE 329

### 7-(4-amino-4,5,6,7-tetrahydro-1-benzothien-2-yl)-8-chloro-1-cyclopropyl-4-oxo-1,4-dihydroquinoline-3-carboxylic acid hydrochloride

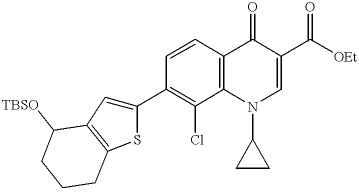

### EXAMPLE 329A

The desired product was prepared by substituting ethyl 7-bromo-8-chloro-1-cyclopropyl-4-oxo-1,4-dihydro-quinoline-3-carboxylate (prepared by the method disclosed in U.S. Pat. No. 6,025,370), Example 38B, and a reaction time of 6 h for ethyl 7-bromo-1-cyclopropyl-4-oxo-1,4-dihydro-3-quinolone-3-carboxylate, Example 1D, and 24 h, respectively, in Example 1 E and was purified by silica gel chromatography eluting with a gradient of 2:1 to 1:1 hexane/acetone to yield an off-white solid.

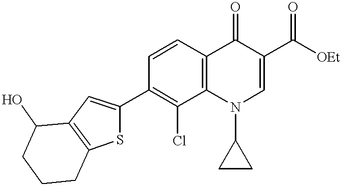

### EXAMPLE 329B

The desired product was prepared by substituting Example 329A for Example 35D in Example 35E to yield a yellow solid.

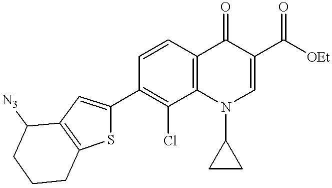

### EXAMPLE 329C

The desired product was prepared by substituting Example 329B for Example 37A in Example 37B and was purified by silica gel chromatography eluting with a gradient of 4:1 to 1:1 hexane/acetone to yield a yellow solid.

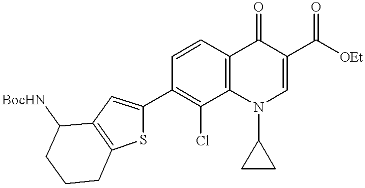

### EXAMPLE 329D

The desired product was prepared by substituting Example 329C for Example 201C in Example 201D and was purified by silica gel chromatography eluting with a gradient of 4:1 to 1:1 hexane/acetone to yield an off-white solid.

### EXAMPLE 329E

### 7-(4-amino-4,5,6,7-tetrahydro-1-benzothien-2-yl)-8-chloro-1-cyclopropyl-4-oxo-1,4-dihydroquinoline-3-carboxylic acid hydrochloride

The desired product was prepared by substituting Example 329D for Example 48C and tetrahydrofuran for ethanol in Example 48D to yield an off-white solid. MS (DCI/NH₃) m/z 415 (M+H)⁺; ¹H NMR (300 MHz, DMSO-d₆) δ 8.93(s, 1H), 8.49 (br s, 2H), 8.35 (d, 1H), 7.77 (s, 1H), 7.75 (d, 1H), 4.46 (m, 2H), 2.86 (m, 2H), 2.13-1.87 (m, 4H), 1.26-1.02 (m, 4H).

### EXAMPLE 330

### 7-(7-amino-4,5,6,7-tetrahydro-1-benzothien-2-yl)-8-chloro-1-cyclopropyl-4-oxo-1,4-dihydroquinoline-3-carboxylic acid hydrochloride

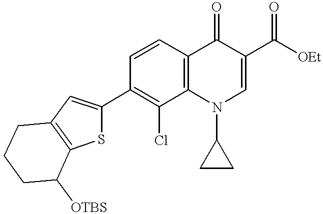

### EXAMPLE 330A

The desired product was prepared by substituting ethyl 7-bromo-8-chloro-1-cyclopropyl-4-oxo-1,4-dihydro-quinoline-3-carboxylate (prepared by the method disclosed in U.S. Pat. No. 6,025,370), Example 41B, and a reaction time of 6 h for ethyl 7-bromo-1-cyclopropyl-4-oxo-1,4-dihydro-3-quinolone-3-carboxylate, Example 1D, and 24 h, respectively, in Example 1E and was purified by silica gel chromatography eluting with a gradient of 2:1 to 1:1 hexane/acetone to yield an off-white solid.

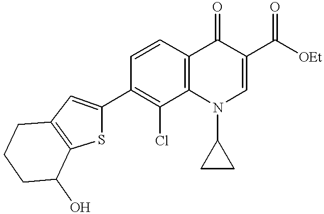

### EXAMPLE 330B

The desired product was prepared by substituting Example 330A for Example 35D in Example 35E to yield a yellow solid.

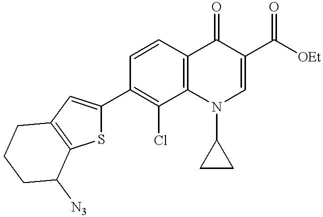

### EXAMPLE 330C

The desired product was prepared by substituting Example 330B for Example 37A in Example 37B and was purified by silica gel chromatography eluting with a gradient of 4:1 to 1:1 hexane/acetone to yield a yellow solid.

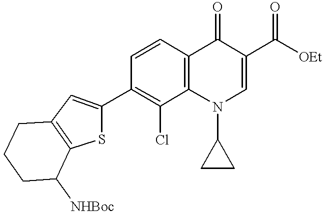

### EXAMPLE 330D

The desired product was prepared by substituting Example 330C for Example 201C in Example 201D and was purified by silica gel chromatography eluting with a gradient of 4:1 to 1:1 hexane/acetone to yield an off-white solid.

### EXAMPLE 330E

### 7-(7-amino-4,5,6,7-tetrahydro-1-benzothien-2-yl)-8-chloro-1-cyclopropyl-4-oxo-1,4-dihydroquinoline-3-carboxylic acid hydrochloride

The desired product was prepared by substituting Example 330D for Example 48C and tetrahydrofuran for ethanol in Example 48D to yield an off-white solid. MS (DCI/NH₃) m/z 415 (M+H)⁺; ¹H NMR (300 MHz, DMSO-d₆) δ 8.93(s, 1H), 8.52 (br s, 2H), 8.34 (d, 1H), 7.78 (d, 1H), 7.38 (s, 1H), 4.63 (m, 1H), 4.46 (m, 1H), 2.77-2.67 (m, 2H), 2.27-1.83 (m, 4H), 1.24-1.06 (m, 4H).

### EXAMPLE 331

### ethyl 7-bromo- 1-cyclopropyl-6-fluoro-8-methyl-4-oxo-1,4-dihydroquinoline-3-carboxylate

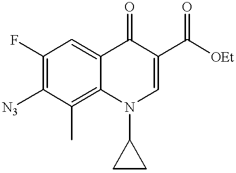

### EXAMPLE 331A

A solution of ethyl 1-cyclopropyl-6,7-difluoro-8-methyl-4-oxo-1,4-dihydro-quinoline-3-carboxylate (7.13 g, 23.2 mmol; prepared by the method of Miyamoto, H.; Ueda, H.; Otsuka, T.; Aki, S., Tamaoka, H.; Chem. Pharm. Bull. 1990, 38, 2472-2475) and sodium azide (6.13 g, 94.3 mmol) in DMF (200 mL) was warmed at 90° C. for 24 h. The heterogeneous mixture was allowed to cool, poured into 500 mL water, filtered, and the solid washed with water and dried in vacuo to provide the desired product as a tan solid (4.46 g, 58%).

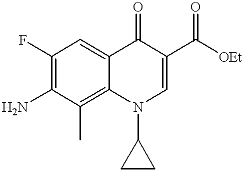

### EXAMPLE 331B

The desired product was prepared by substituting Example 33 1A for Example 266A in Example 266B.

### EXAMPLE 331C

### ethyl 7-bromo-1-cyclopronyl-6-fluoro-8-methyl-4-oxo-1,4-dihydroquinoline-3-carboxylate

The desired product was prepared by substituting Example 33 1B for Example 266B in Example 266C. MS (DCI) m/z 368, 370 (M+H)⁺; ¹H NMR (CDCl₃, 300 MHz) δ 8.68 (s, 1 H), 8.03 (d, 1H), 4.39 (q, 2H), 3.98 (m, 1 H), 2.92 (s, 3 H), 1.41 (t, 3 H), 1.21 (m, 2 H), 0.89 (m, 2 H).

### EXAMPLE 332

### ethyl 5-(acetylamino)-7-bromo-1-cyclopropyl-6,8-difluoro-4-oxo-1,4-dihydroquinoline-3-carboxylate

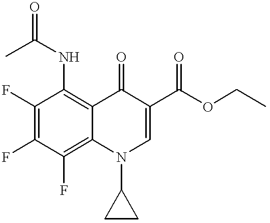

### EXAMPLE 332A

A solution of ethyl 5-amino-1-cyclopropyl-6,7,8-trifluoro-4-oxo-1,4-dihydro-quinoline-3-carboxylate (Miyamoto, T., Matsumoto, J., Chiba, K., Egawa, H., Shibamori, K. J. Med. Chem. 1990, 33, 1645-1656.) (7.63 g, 23.4 mmol) in glacial acetic acid (150 ml) was treated with acetic anhydride (11.0 ml, 0.117 mol) and heated to 90° C. for 3 h. The reaction mixture was concentrated to dryness, partitioned between water and CHCl₃ and neutralized by the addition of 1 M NaOH. The layers were separated, the aqueous layer extracted with CHCl₃ and the combined organic layers washed with water, brine and dried (MgSO₄). Concentratoin gave the desired product (8.60 g, 1 00%) as a yellow solid that was used without further purification.

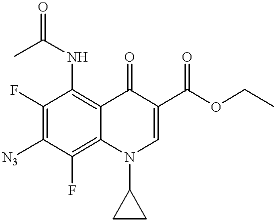

### EXAMPLE 332B

The desired product was prepared by substituting Example 332A for ethyl 1-cyclopropyl-6,7-difluoro-4-oxo-1,4-dihydro-quinoline-3-carboxylate in Example 266A.

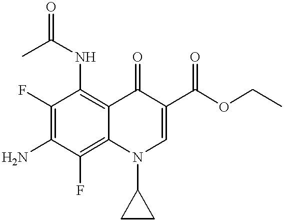

### EXAMPLE 332C

The desired product was prepared by substituting Example 332B for Example 266A in Example 266B.

### EXAMPLE 332D

### ethyl 5-(acetylamino)-7-bromo-1-cyclopropyl-6,8-difluoro-4-oxo-1,4-dihydroquinoline-3-carboxylate

A suspension of Example 332C (5.92 g, 16.20 mmol) in 5% aqueous HBr (350 ml) was treated with cupric bromide (18.10 g, 81.02 mmol), cooled to 0° C., treated dropwise with a solution of sodium nitrite (2.24 g, 32.41 mmol) in water (20 ml), was allowed to warm to room temperature and stir for 12 h. The reaction mixture was diluted to a total volume of 1.8 L with water, stirred for 30 minutes and filtered. The resulting solid was rinsed with water, hexanes and dried to provide the desired product (4.93 g, 71 %) as a colorless solid. MS (DCI/NH₃) m/z 429 (M+H)^{+;} ¹H NMR (300 MHz, DMSO-d₆) δ 10.82 (s, 1H), 8.47 (s, 1H), 4.24 (q, 2H), 4.03 (m, 1H), 2.13 (s, 3H), 1.28 (t, 3H), 1.13 (m, 4H).

### EXAMPLE 333

### 5-amino-7-(4-amino4,5,6,7-tetrhydro-1-benzothien-2-yl)-1-cyclopropyl-6,8-difluoro4-oxo- 1,4-dihydroquinoline-3-carboxylic acid hydrochloride

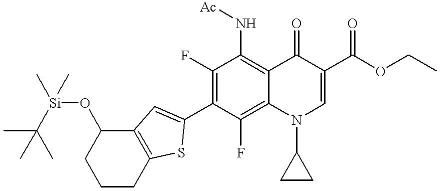

### EXAMPLE 333A

The desired product was prepared by substituting Example 332D for Example 266C in Example 296A.

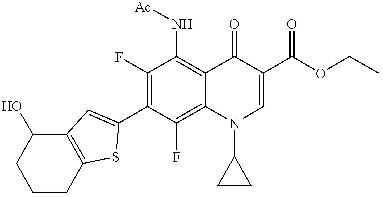

### EXAMPLE 333B

The desired product was prepared by substituting Example 333A for Example 35D in Example 35E and was used with fifther purification.

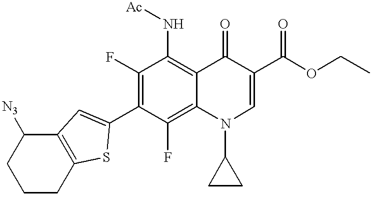

### EXAMPLE 333C

The desired compound was prepared by substituting Example 333B for Example 37A in Example 37B and was purified by silica gel chromatography eluting with 2% methanol/dichloromethane to yield a yellow solid.

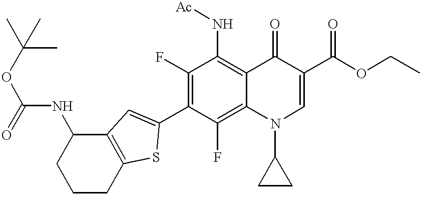

### EXAMPLE 333D

The desired compound was prepared by substituting Example 333C for Example 201C in Example 201D and was purified by silica gel chromatography eluting with 2% methanol/dichloromethane to yield a yellow solid.

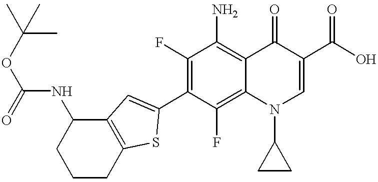

### EXAMPLE 333E

A solution of the 333D (0.26 g, 0.40 mmol) in 1:1 tetrahydrofuran: 5% sodium hydroxide (10 mL) was heated at 70° C. for 24h, allowed to cool to ambient temperature, was poured into water (100 mL) and adjusted to pH 3 with conc HCl. The reaction mixture was extracted with dichloromethane, the organic phase dried (Na₂SO₄) and concentrated to yield the desired product as a yellow solid (0.22 g, 96%)

### EXAMPLE 333F

### 5-amino-7-(4-amino-4,5,6,7-tetrahydro-1-benzothien-2-yl)-1-cyclopropyl-6,8-difluoro-4-oxo-1,4-dihydroquinoline-3-carboxylic acid hydrochloride

The desired compound was prepared by substituting Example 333E for Example 40B in Example 40C and was purified by trituration with diethyl ether. MS (DCI/NH₃) m/z 432 (M+H)⁺¹H NMR (300 MHz, DMSO) δ 8.62 (s, 1H), 8.44 (m, 2H), 7.79 (s, 1H), 7.49 (m, 2H), 4.46 (m, 1H), 4.11 (m, 1H), 4.11 (m, 1H),2.88 (m 2H), 1.96-2.20 (m, 2H), 1.86 (m, 2H), 1.15 (m, 4H).

### EXAMPLE 334

### 7-(7-amino-4,5,6,7-tetrahydro-1-benzothien-2-yl)-1-cyclopropyl-6-methyl-4-oxo-1,4-dihydro-1,8-naphthyridine-3-carboxylic acid hydrochloride

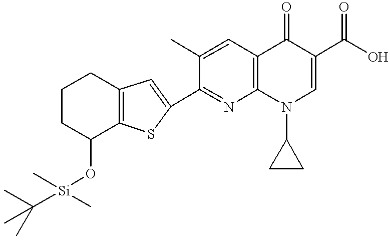

### EXAMPLE 334A

The desired product was prepared by substituting Example 335E and Example 41B for ethyl 7-bromo-1-cyclopropyl-4-oxo-1,4-dihydro-3-quinolone-3-carboxylate and Example 1D, respectively, in Example 1E with a reaction time of 7 h.

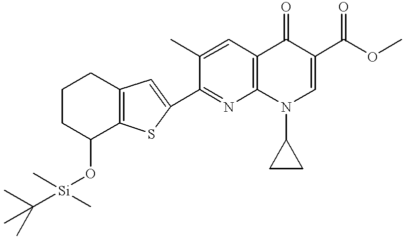

### EXAMPLE 334B

The desired product was prepared by substituting Example 334A for 38C in Example 39A to yield a solid (0.65 g, 77%).

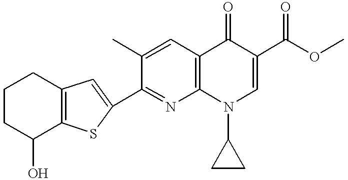

### EXAMPLE 334C

The desired compound was prepared by substituting Example 334B for Example 35D in Example 35E and was purified by silica gel chromatography eluting with 2% methanol/dichloromethane to yield a yellow solid.

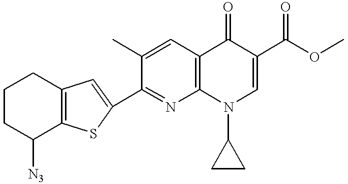

### EXAMPLE 334D

The desired compound was prepared by substituting Example 334C for Example 37A in Example 37B and was purified by silica gel chromatography eluting with 2% methanol/dichloromethane to yield a yellow solid.

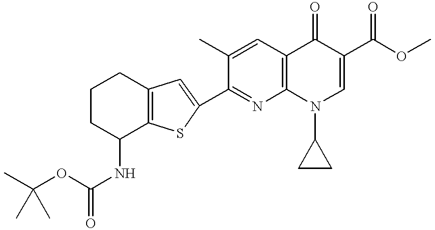

### EXAMPLE 334E

The desired compound was prepared by substituting Example 334D for Example 201C in Example 201D to yield a yellow solid that was used without purification.

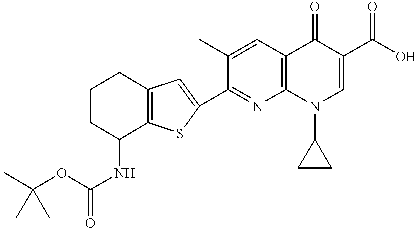

### EXAMPLE 334F

The desired compound was prepared by substituting Example 334E for Example 2A in Example 2B to yield a yellow solid that was used without purification.

### EXAMPLE 334G

### 7-(7-amino-4,5,6,7-tetrahydro-1-benzothien-2-yl)-1-cyclopropyl-6-methyl-4-oxo-1,4-dihydro-1,8-naphthyridine-3-carboxylic acid hydrochloride

The desired compound was prepared by substituting Example 334F for Example 40B in Example 40C and was purified by trituration with diethyl ether to yield a solid. MS (DCI/NH₃) m/z 396 (M+H)⁺¹H NMR (300 MHz, DMSO) δ 8.82 (s, 1H), 8.62 (s, 1H), 8.45 (m, 2H), 7.22 (s, 1H0, 4.65 (m, 1H), 3.81 (m, 1H), 2.76 (s, 3H), 2.72 (m, 2H), 2.13 (m, 1H), 1.96 (m, 2H), 1.82 (m, 1H), 1.33 (m, 2H), 1.20 (m, 2H).

### EXAMPLE 335

### ethyl 7-chloro-1-cyclopropyl-6-methyl-4-oxo-1,4-dihydro-1,8-naphthyridine-3-carboxylate

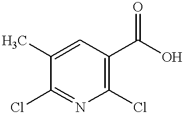

### EXAMPLE 335A

A solution of methyl 2,6-dichloro-5-methyl-nicotinate (prepared by the method of Meerpoel, Hoomaert, et al. Tetrahedron Letters 1989, 30, 3183-3186) (4.43 g, 20.10 mmol) in methanol (60 mL) was treated with 2.5M sodium hydroxide (15 mL, 37.50 mmol) and was stirred at ambient temperature for 3 h. The solvent was removed under vacuum, the residue diluted with water and washed with hexanes to remove impurities. The aqueous layer was adjusted to pH 2 with HCl and was extracted with dichloromethane, the organic phase dried (Na₂SO₄), and concentrated to yield a white solid (2.21 g, 53%)

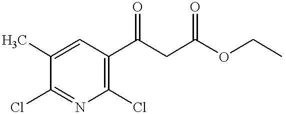

### EXAMPLE 335B

The desired product was prepared by substituting Example 335A for Example 269A in Example 269B to yield a tan solid that was used without purification.

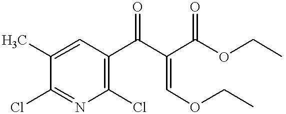

### EXAMPLE 335C

The desired product was prepared by substituting Example 335B for Example 269B in Example 269C to yield an oil that was used without purification.

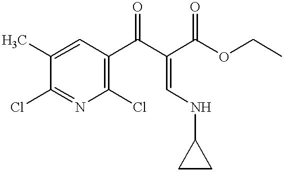

### EXAMPLE 335D

The desired product was prepared by substituting Example 335C for Example 269C in Example 269D to yield a dark tan solid that was used with out purification.

### EXAMPLE 335E

### ethyl 7-chloro-1-cyclopropyl-6-methyl-4-oxo-1,4-dihydro-1,8-naphthyridine-3-carboxylate

The desired product was prepared by substituting Example 335D and acetonitrile for Example 269D and N,N-dimethylformamide, respectively, in Example 269E and was purified by silica gel chromatography eluting with 1% methanol/dichloromethane to yield a tan solid. MS (DCI/NH₃) m/z 307 (M+H)⁺¹H NMR (300 MHz, CDCl₃) δ 8.62 (s, 1H), 8.54 (s, 1H), 4.40 (q, 2H), 3.66 (m, 1H), 2.50 (s, 3H), 1.41 (t, 3H), 1.30 (m, 2H), 1.04 (m, 2H).

### EXMAPLE 336

### 7-(7-(benzylamino)-4,5,6,7-tetrahydro-1-benzothien-2-yl)-1-cyclopropyl-8-methoxy-4-oxo-1,4-dihydroquinoline-3-carboxylic acid

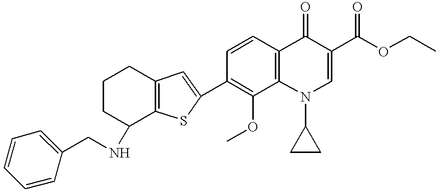

### EXAMPLE 336A

The desired product was prepared by substituting Example 302A and benzaldehyde for Example 83A and 3-pyridine carboxaldehyde, respectively, in Example 94A.

### EXAMPLE 336B

### 7-(7-(benzylamino)-4,5,6,7-tetrahydro-1-benzothien-2-yl)-1-cyclopropyl-8-methoxy-4-oxo-1,4-dihydroquinoline-3-carboxylic acid

The desired product was prepared by substituting Example 336A for Example 2A in Example 2B. MS(ESI)m/z 501 (M+H)⁺; ¹H NMR (300 MHz, DMSO-d⁻₆) δ 8.78 (s, 1H), 8.07 (d, 1H), 7.83(b, 1H), 7.52(m, 2H), 7.45(m, 1H), 7.35(m, 2H), 7.24(m, 1H), 4.20(b, 1H), 3.80(m, 2H), 3.75(m, 1H), 3.60(m, 4H), 2.60(m, 2H), 2.15(m, 1H), 2.00(m, 1H), 1.65(m, 2H), 1.13(m, 2H), 0.97(m, 2H).

### EXAMPLE 337

### 1-cyclopropyl-8-methoxy-4-oxo-7-(7-((pyridin-3-ylmethyl)amino)-4,5,6,7-tetrahydro-1-benzothien-2-yl)-1,4-dihydroquinoline-3-carboxylic acid

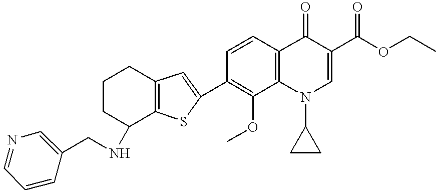

### EXAMPLE 337A

The desired product was prepared by substituting Example 302A for Example 83A in Example 94A.

### EXAMPLE 337B

### 1-cyclopropyl-8-methoxy-4-oxo-7-(7-((pyridin-3-ylmethyl)amino)-4,5,6,7-tetrahydro-1-benzothien-2-yl)-1,4-dihydroquinoline-3-carboxylic acid

The desired product was obtained the same as in Example 336B to obtain 0.388 g of Example 337B. MS(ESI) 502 (M+H)⁺; ¹H NMR (DMSO-d⁻₆) 8.78(s, 1H), 8.64(d.1H), 8.42(m, 1H), 8.10(d, 1H), 7.97(m, 1H), 7.84(m, 1H), 7.56(d, 1H), 7.34(m, 1H), 4.25(m, 1H), 3.94(m, M1), 3.65(m, 4H), 2.63(m, 2H), 2.40 (m, 1H), 2.16 (m, 1H), 2.00(2H), 1.68(m, 2H), 1.16(m, 2H), 1.04(m, 2H).

### EXAMPLE 338

### 7-(4-amino-4,5,6,7-tetrahydro-1-benzothien-2-yl)-1-cyclopropyl-6-methyl-4-oxo-1,4-dihydroquinoline-3-carboxylic acid hydrochloride

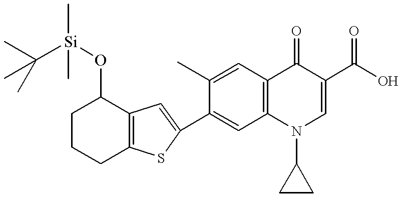

### EXAMPLE 338A

The desired product was prepared by substituting Example 269E and Example 38B for ethyl 7-bromo-1-cyclopropyl-4-oxo-1,4-dihydro-3-quinolone-3-carboxylate and Example 1D, respectively, in Example 1E and was purified by silica gel chromatography to yield a solid.

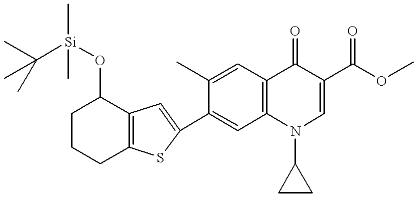

### EXAMPLE 338B

The desired product was prepared by substituting Example 338A for 38C in Example 39A to yield a solid (1.15 g, 100%) that was used without purification.

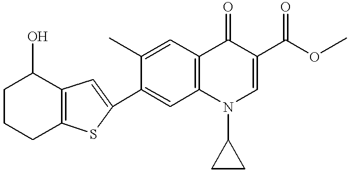

### EXAMPLE 338C

The desired compound was prepared by substituting Example 338B for Example 35D in Example 35E and was used without purification.

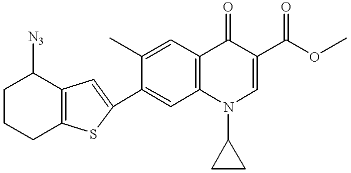

### EXAMPLE 338D

The desired compound was prepared by substituting Example 338C for Example 37A in Example 37B and was purified by silica gel chromatography eluting with 2% methanol/dichloromethane to yield a yellow solid.

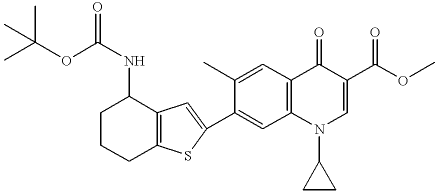

### EXAMPLE 338E

The desired compound was prepared by substituting Example 338D for Example 201C in Example 201D and was purified with silica gel chromatography eluting with 2% methanol/dichloromethane to yield a yellow solid.

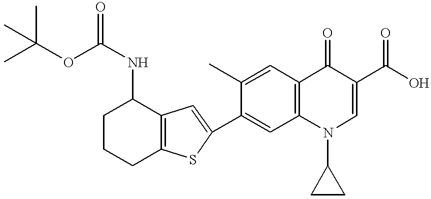

### EXAMPLE 338F

The desired compound was prepared by substituting Example 338E for Example 2A in Example 2B to yield a yellow solid that was used without purification.

### EXAMPLE 338G

### 7-(4-amino-4,5,6,7-tetrahydro-1-benzothien-2-yl)-1-cyclopropyl-6-methyl-4-oxo-1,4-dihydroquinoline-3-carboxylic acid hydrochloride

The desired compound was prepared by substituting Example 338F for Example 40B in Example 40C and was purified by trituration with diethyl ether to yield a solid. MS (DCI/NH₃) n/z 395 (M+H)⁺; ¹H NMR (300 MHz, DMSO-d₆) δ 8.76 (s, 1H), 8.35 (m, 2H), 8.31 (s, 1H), 8.18 (s, 1H), 7.58 (s, 1H), 4.45 (m, 1H), 3.86 (m, 1H), 2.87 (m, 2H), 2.63 (s, 3H), 2.20 (m, 2H), 1.87 (m, 2H), 1.32 (m, 2H), 1.22 (m, 2H).

### EXAMPLE 339

### 1-cyclopropyl-7-(4-hydroxy-4,5,6,7-tetrahydro-1-benzothien-2-yl)-6-methyl-4-oxo-1,4-dihydroquinoline-3-carboxylic acid

The desired product was prepared by substituting Example 338A for Example 35D in Example 35E and was purified by trituration with diethyl ether to yield a yellow solid. MS (DCI/NH₃) m/z 396 (M+H)⁺; ¹H NMR (300 MHz, DMSO-d₆) δ 15.13 (s, 1H), 8.73 (2, 1H), 8.27 (s, 1H), 8.18 (s, 1H), 7.34 (s, 1H), 4.63 (m, 1H), 3.90 (m, 1H), 2.78 (m, 2H), 2.62 (s, 3H), 1.95 (m, 2H), 1.75 (m, 2H), 1.30 (m, 2H), 1.20 (m, 2H).

### EXAMPLE 340

### 7-(4-amino-4,5,6,7-tetrahydro-1-benzothien-2-yl)-1-cyclopropyl-6-methyl-4-oxo-1,4-dihydro-1,8-naphthyridine-3-carboxylic acid hydrochloride

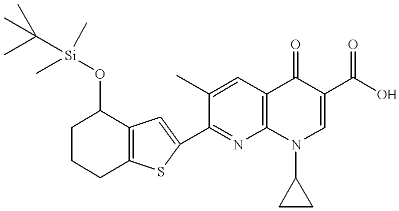

### EXAMPLE 340A

The desired product was prepared by substituting Example 335E and Example 38B for ethyl 7-bromo-1-cyclopropyl-4-oxo-1,4-dihydro-3-quinolone-3-carboxylate and Example 1D, respectively, in Example 1E with a reaction time of 7h and was purified by silica gel chromatography to yield a solid.

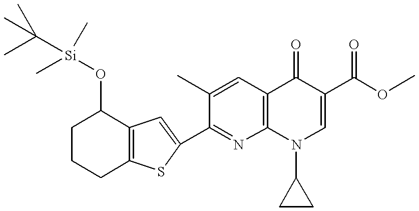

### EXAMPLE 340B

The desired product was prepared by substituting Example 340A for 38C in Example 39A and was purified by silica gel chromatography eluting with 2% methanol/dichloromethane to yield a solid.

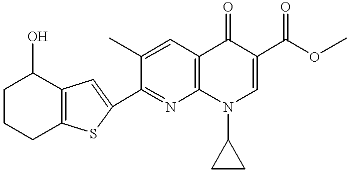

### EXAMPLE 340C

The desired compound was prepared by substituting Example 340B for Example 35D in Example 35E and was purified by silica gel chromatography eluting with 2% methanol/dichloromethane to yield a yellow solid.

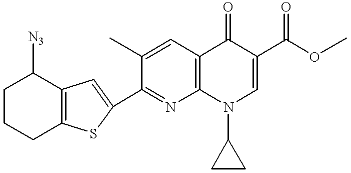

### EXAMPLE 340D

The desired compound was prepared by substituting Example 340C for Example 37A in Example 37B and was purified by silica gel chromatography eluting with 2% methanol/dichloromethane to yield a yellow solid.

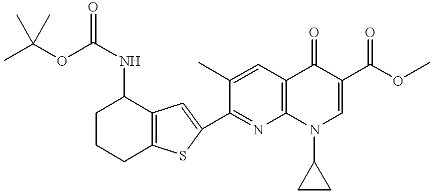

### EXAMPLE 340E

The desired compound was prepared by substituting Example 340D for Example 201C in Example 201D and was purified with silica gel chromatography eluting with 2% methanol/dichloromethane to yield a yellow solid.

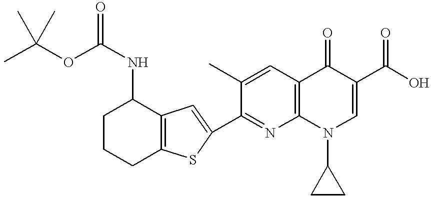

### EXAMPLE 340F

The desired compound was prepared by substituting Example 340E for Example 2A in Example 2B to yield a yellow solid that was used without purification.

### EXAMPLE 340G

### 7-(4-amino-4,5,6,7-tetrahydro-1-benzothien-2-yl)-1-cyclopropyl-6-methyl-4-oxo-1,4-dihydro-1,8-naphthyridine-3-carboxylic acid hydrochloride

The desired compound was prepared by substituting Example 340F for Example 40B in Example 40C and was purified by trituration with diethyl ether to yield a solid. MS (DCI/NH₃) m/z 396 (M+H)⁺; ¹H NMR (300 MHz, DMSO-d₆) δ 8.1 (s, 1H), 8.60 (s, 1HO, 8.35 (m, 2H), 8.07 (s, 1H), 4.45 (m, 1H), 2.83 (m, 1H), 2.86 (m, 2H), 2.77 (s, 3H), 1.80-2.18 (m, 4H).

### EXAMPLE 341

### 7-(7-amino-6,6-dimethyl-4,5,6,7-tetrahydro-1-benzothien-2-yl)-1-cyclopropyl-6-fluoro-4-oxo-1,4-dihydroquinoline-3-carboxylic acid hydrochloride

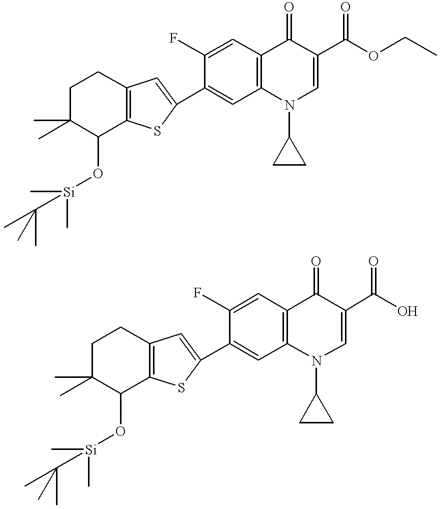

### EXAMPLE 341A EXAMPLE 342A

The desired products were prepared by substituting Example 266C and Example 241C for ethyl 7-bromo-1-cyclopropyl-4-oxo-1,4-dihydro-3-quinolone-3-carboxylate and Example 1D, respectively, in Example 1E with a reaction time of 2.5h and was purified by silica gel chromatography eluting with 2% methanol and 0.5% ammonium hydroxide.

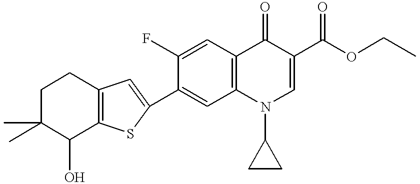

### EXAMPLE 341B

The desired compound was prepared by substituting Example 341A for Example 35D in Example 35E to yield a yellow solid that was used without purification.

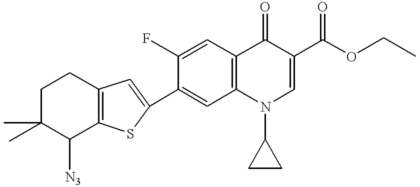

### EXAMPLE 341C

The desired compound was prepared by substituting Example 341B for Example 37A in Example 37B and was purified by silica gel chromatography eluting with 2% methanol/dichloromethane to yield a yellow solid.

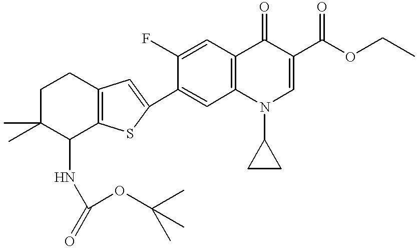

### EXAMPLE 341D

The desired compound was prepared by substituting Example 341C for Example 201C in Example 201D to yield a yellow solid that was used without purification.

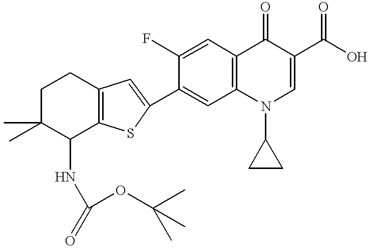

### EXAMPLE 341E

The desired product was prepared by substituting Example 341D for Example 2A in Example 2B and was used without purification.

### EXAMPLE 341F

### 7-(7-amino-6,6-dimethyl-4,5,6,7-tetrahydro-1-benzothien-2-yl)-1-cyclopropyl-6-fluoro-4-oxo-1,4-dihydroquinoline-3-carbolxylic acid hydrochloride

The desired compound was prepared by substituting Example 341E for Example 40B in Example 40C and was purified by trituration with 1:2 ethanol: diethyl ether to yield a solid. MS (DCI/NH₃) m/z 427 (M+H)⁺; ¹H NMR (300 MHz, DMSO-d₆) δ 8.78 (s, 1H), 8.55 (d, 1H), 8.35 (m, 2H), 8.17 (d, 1H), 7.73 (s, 1H), 4.32 (m, 1H), 3.98 (m, 1H), 2.73 (m, 2H), 1.89 (m, 1H), 1.62 (m, 1H), 1.37 (m, 2H), 1.26 (m, 2H), 1.08 (s, 6H).

### EXAMPLE 342

### 1-cyclopropyl-6-fluoro-7-(7-hydroxy-6,6-dimethyl-4,5,6,7-tetrahydro-1-benzothien-2-yl)-4-oxo-1,4-dihydroquinoline-3-carboxylic acid

### EXAMPLE 342B

The desired compound was prepared by substituting Example 342A in Example 341A for Example 35D in Example 35E and was purified by trituration in diethyl ether to yield a yellow solid. MS (DCI/NH₃) m/z 428 (M+H)⁺; ¹H NMR (300 MHz, DMSO-d₆) δ 14.84 (s, 1H), 8.76 (s, 1H), 8.48 (d, 1H), 8.11 (d, 1H), 7.56 (s, 1H), 5.51 (d, 1H), 4.38 (m, 1H), 3.96 (m, 1H), 2.63 (m, 2H), 1.72 (m, 2H), 1.36 (m, 2H), 1.23 (m, 2H), 1.03 (s, 3H), 0.88 (s, 3H).

### EXAMPLE 343

### 7-(7-amino-6,6-dimethyl-4,5,6,7-tetrahydro-1-benzothien-2-yl)-1-cyclopropyl-6-fluoro-4-oxo-1,4-dihydro-1,8-naphthyridine-3-carboxylic acid hydrochloride

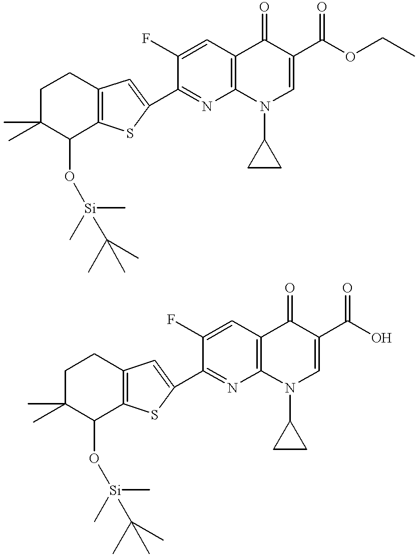

### EXAMPLE 343A EXAMPLE 344A

The desired products were prepared by substituting ethyl 7-chloro-1-cyclopropyl-6-fluoro-4-oxo-1,4-dihydro-[1,8]naphthyridine-3-carboxylate and Example 241C for ethyl 7-bromo-1-cyclopropyl-4-oxo-1,4-dihydro-3-quinolone-3-carboxylate and Example 1D, respectively, in Example IE with a reaction time of 2.5h and was purified by silica gel chromatography eluting with 2% methanol and 0.5% ammonium hydroxide.

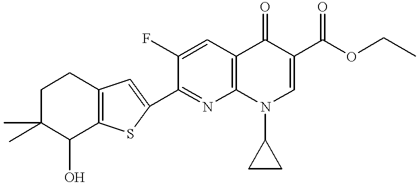

### EXAMPLE 343B

The desired compound was prepared by substituting Example 343A for Example 35D in Example 35E to yield a yellow solid that was used without purification.

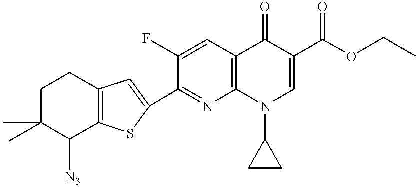

### EXAMPLE 343C

The desired compound was prepared by substituting Example 343B for Example 37A in Example 37B and was purified by silica gel chromatography eluting with 2% methanol/dichloromethane to yield a yellow solid.

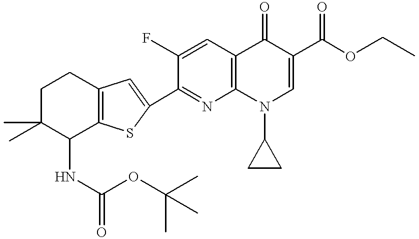

### EXAMPLE 343D

The desired compound was prepared by substituting Example 343C for Example 201C in Example 201D and was purified by silica gel chromatography eluting with 2% methanol/dichloromethane to yield a yellow solid.

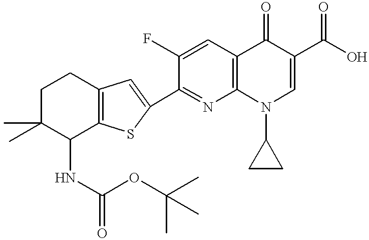

### EXAMPLE 343E

The desired product was prepared by substituting Example 343D for Example 2A in Example 2B and was used without purification.

### EXAMPLE 343F

### 7-(7-amino-6,6-dimethyl-4,5,6,7-tetrahydro-1-benzothien-2-yl)-1-cyclopropyl-6-fluoro-4-oxo-1,4-dihydro-1,8-naphthyridine-3-carboxylic acid hydrochloride

The desired compound was prepared by substituting Example 343E for Example 40B in Example 40C and was purified by trituration with 1:2 ethanol: diethyl ether. MS (DCI/NH₃) m/z 428 (M+H)⁺; ¹H NMR (300 MHz, DMSO-d₆) δ 8.85 (s, 1H0, 8.60 (d, 1H), 8.40 (m, 2H), 7.86 (d, 1H), 4.35 (m, 1H), 3.82 (m, 1H), 2.73 (m, 2H), 1.88 (m, 1H), 1.64 (m, 1H), 1.33 (m, 2H), 1.22 (m, 2H), 1.05 (s, 3H), 1.07 (s, 3H).

### EXAMPLE 344

### 1-cyclopropyl-6-fluoro-7-(7-hydroxy-6,6-dimethyl-4,5,6,7-tetrahydro-1-benzothien-2-yl)-4-oxo-1,4-dihydro-1,8-naphthyridine-3-carboxylic acid

### EXAMPLE 344B

The desired compound was prepared by substituting Example 344A for Example 35D in Example 35E and was purified by trituration in diethyl ether to yield a yellow solid. MS (DCI/NH₃) m/Z 429 (M+H)⁺; ¹H NMR (300 MHz, DMSO-d₆) δ 8.81 (s, 1H), 8.51 (s, 1H), 7.75 (d, 1H), 5.67 (d, 1H), 4.40 (d, 1H), 3.85 (m, 1H), 2.63 (m, 2H), 1.67 (m, 2H), 1.28 (m, 2H), 1.19 (m, 2H), 1.03 (s, 3H), 0.87 (s, 3H).

### EXAMPLE 345

### 5-amino-7-(7-amino-4,5,6,7-tetrahydro-1-benzothien-2-yl)-1-cyclopropyl-6,8-difluoro-4-oxo-1,4-dihydroquinoline-3-carboxylic acid hydrochloride

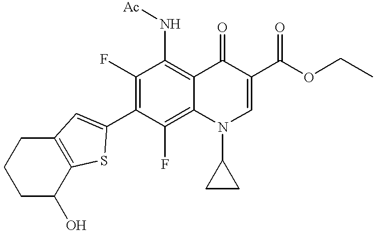

### EXAMPLE 345A

The desired product was prepared by substituting Example 319A for Example 35D in Example 35E and was used without furrther purification.

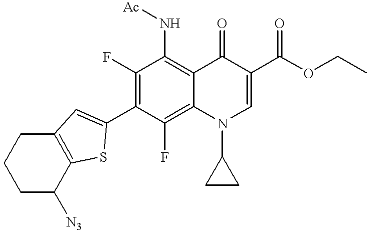

### EXAMPLE 345B

The desired compound was prepared by substituting Example 345A for Example 37A in Example 37B and was purified by silica gel chromatography eluting with 2% methanol/dichloromethane to yield a yellow solid.

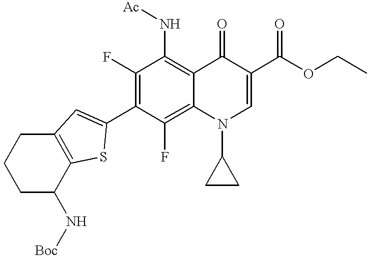

### EXAMPLE 345C

The desired compound was prepared by substituting Example 345B for Example 201C in Example 201D and was purified by silica gel chromatography eluting with 2% methanol/dichloromethane to yield a yellow solid.

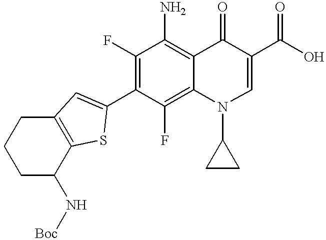

### EXAMPLE 345D

The desired product was prepared by substituting Example 345C for Example 333D in Example 333E and was used without purification.

### EXAMPLE 345E

### 5-amino-7-(7-amino-4,5,6,7-tetrahydro-1-benzothien-2-yl)-1-cyclopropyl-6,8-difluoro-4-oxo-1,4-dihydroquinoline-3-carboxylic acid hydrochloride

The desired compound was prepared by substituting Example 345D for Example 40B in Example 40C and was purified by trituration with diethyl ether to yield a solid (0.38 g, 83%). MS (DCI/NH₃) m/z 432 (M+H)⁺; ¹H NMR (300 MHz, DMSO-d₆) δ 8.63 (s, 1H), 8.45 (m, 2H), 7.49 (m, 2H), 7.38 (s, 1H), 4.65 (m, 1H), 4.09 (m, 1H), 2.71 (m, 2H), 2.13 (m, 1H), 1.98 (m, 2H) 1.83 (m, 1H), 1.14 (m, 4H).

### EXAMPLE 346

### 5-amino-1-cyclopropyl-6,8-difluoro-7-(4-(methylamino)-4,5,6,7-tetrahydro-1-benzothien-2-yl)-4-oxo-1,4-dihydroquinoline-3-carboxylic acid hydrochloride

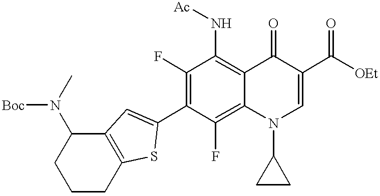

### EXAMPLE 346A

The desired product was prepared by substituting Example 332D and Example 210B for Example 266C and Example 38B, respectively in Example 296A and was purified by silica gel chromatography eluting with 33% acetone in hexanes.

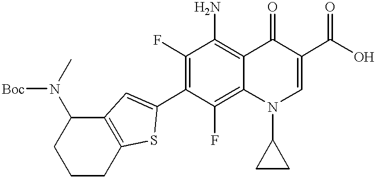

### EXAMPLE 346B

The desired product was prepared by substituting Example 346A for Example 333D in Example 333E and was used without further purification.

### EXAMPLE 346C

### 5-amino-1-cyclopropyl-6,8-difluoro-7-(4-(methylamino)-4,5,6,7-tetrahydro-1-benzothien-2-yl)-4-oxo-1,4-dihydroquinoline-3-carboxylic acid hydrochloride

The desired product was prepared by substituting Example 346B for Example 40B in Example 40C. MS (DCI/NH₃) m/z 446 (M+H)⁺; ¹H NMR (300 MHz, DMSO-d₆) δ 9.18 (br m, 1H), 9.09 (br m, 1H), 8.62 (s, 1H), 7.84 (s, 1H), 7.49 (br s, 2H), 4.45 (m, 1H), 4.09 (m, 1H), 2.90 (m, 2H), 2.62 (br t, 3H), 2.25-1.95 (m, 2H), 1.92-1.78 (m, 2H), 1.14 (m, 4H).

### EXAMPLE 347

### 7-(7-amino-6,6-dimethyl-4,5,6,7-tetrahydro-1-benzothien-2-yl)-8-chloro-1-cyclopropyl-4-oxo-1,4-dihydroquinoline-3-carboxylic acid hydrochloride

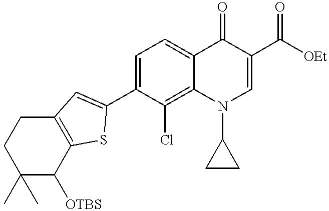

### EXAMPLE 347A

The desired product was prepared by substituting ethyl 7-bromo-8-chloro-1-cyclopropyl-4-oxo-1,4-dihydro-quinoline-3-carboxylate (prepared by the method disclosed in U.S. Pat. No. 6,025,370) and Example 241C for Example 266C and Example 38B in Example 296A and was purified by silica gel chromatography eluting with 33% acetone in hexanes.

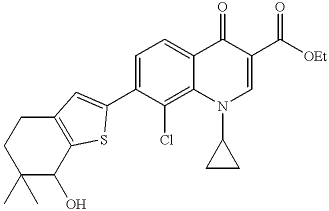

### EXAMPLE 347B

The desired product was prepared by substituting Example 347A for Example 35D in Example 35E and was used without further purification.

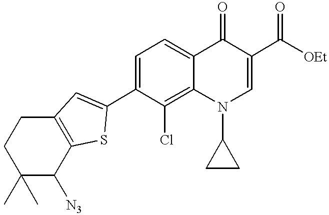

### EXAMPLE 347C

The desired product was prepared by substituting Example 347B for Example 37A in Example 37B and was purified by silica gel chromatography eluting with 25% then 33% acetone in hexanes.

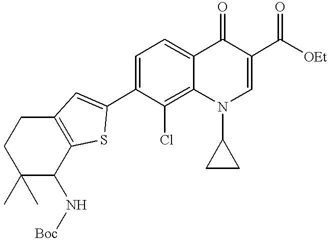

### EXAMPLE 347D

The desired product was prepared by substituting Example 347C for Example 201C in Example 201D and was purified by silica gel chromatography eluting with 25% acetone in hexanes.

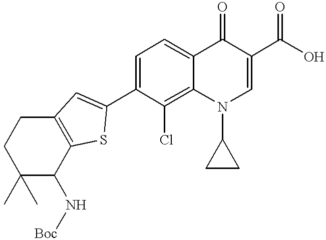

### EXAMPLE 347E

The desired product was prepared by substituting Example 347D for Example 296B in Example 296C and was used without further purification.

### EXAMPLE 347F

### 7-(7-amino-6,6-dimethyl-4,5,6,7-tetrahydro-1-benzothien-2-yl)-8-chloro-1-cyclopropyl-4-oxo-1,4-dihydroquinoline-3-carboxylic acid hydrochloride

The desired product was prepared by substituting Example 347E for Example 40B in Example 40C. MS (DCI/NH₃) m/z 443 (M+H)⁺; ¹H NMR (300 MHz, DMSO-d₆) δ 8.93 (s, 1H), 8.44 (br s, 3H), 8.33 (d, 1H), 7.87 d, 1H), 7.41 (s, 1H), 4.46 (m, 1H), 4.29 (m, 1H), 2.82-2.58 (m, 2H), 1.88 (m, 1H), 1.62 (m, 1H), 1.23 (m, 2H), 1.08 (s, 6H), 1.06 (m, 2H).

### EXAMPLE 348

### 7-(7-amino-6,6-dimethyl-4,5,6,7-tetrahydro-1-benzothien-2-yl)-1-cyclopropyl-6-fluoro-8-methoxy-4-oxo-1,4-dihydroquinoline-3-carboxylic acid hydrochloride

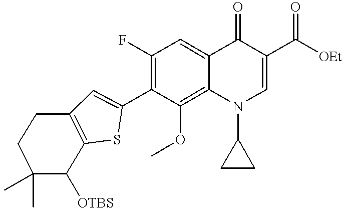

### EXAMPLE 348A

The desired product was prepared by substituting Example 374G and Example 241C for Example 266C and Example 38B in Example 296A and was purified by silica gel chromatography eluting with 33% acetone in hexanes.

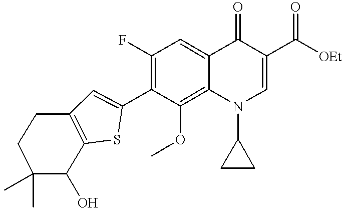

### EXAMPLE 348B

The desired product was prepared by substituting Example 348Afor Example 35D in Example 35E and was used without further purification.

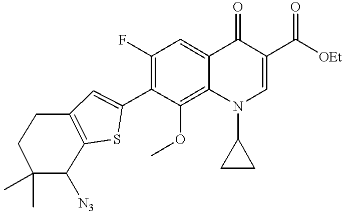

### EXAMPLE 348C

The desired product was prepared by substituting Example 348B for Example 37A in Example 37B and was purified by silica gel chromatography eluting with 25% then 33% acetone in hexanes.

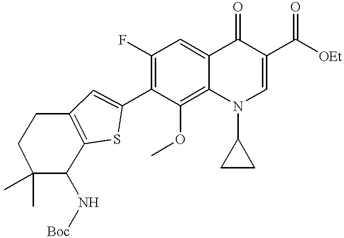

### EXAMPLE 348D

The desired product was prepared by substituting Example 348C for Example 201C in Example 201D and was purified by silica gel chromatography eluting with 25% then 33% acetone in hexanes.

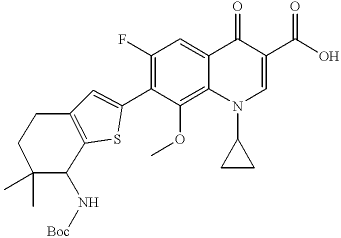

### EXAMPLE 348E

The desired product was prepared by substituting Example 348D for Example 296B in Example 296C and was used without purification.

### EXAMPLE 348F

### 7-(7-amino-6,6-dimethyl-4,5,6,7-tetrahydro-1-benzothien-2-yl)-1-cyclopropyl-6-fluoro-8-methoxy-4-oxo-1,4-dihydroquinoline-3-carboxylic acid hydrochloride

The desired product was prepared by substituting Example 348E for Example 40B in Example 40C. MS (DCI/NH₃) m/z 457 (M+H)⁺; ¹H NMR (300 MHz, DMSO-d₆) δ 8.82 (s, 1H), 8.41 (br s, 3H), 7.95 (d, 1H), 7.44 (d, 1H), 4.31 (m, 1H), 4.24 (m, 1H), 3.66 (s, 3H), 2.82-2.58 (m, 2H), 1.88 (m, 1H), 1.63 (m, 1H), 1.21-1.03 (m, 4H), 1.09 (s, 6H).

### EXAMPLE 349

### 5-amino-7-(7-amino-4,5,6,7-tetrahydro-1-benzothien-2-yl)-1-cyclopropyl-8-fluoro-4-oxo-1,4-dihydroquinoline-3-carboxylic acid trifluoroacetic acid salt

A solution of Example 326F (150 mg, 0.33 mmol) in ammonium hydroxide (6 mL) was heated in a sealed tube at 100° C. for 72 hours, cooled and lyopholyzed. The crude product was purified by C18 reverse phase flash chromatography eluting with 1:1:0.01 water/acetonitrile/TFA to give the desired product as a light brown solid (20 mg, 14%). MS (DCI) mlz 414 (M+H)⁺; ¹H NMR (300 MHz, DMSO-d₆) δ 8.59 (s, 1H), 8.38 (bs, 3H), 7.57 (bs, 2H), 7.38 (s, 1H), 6.95 (d, 1H), 4.62 (m, 1H), 4.11 (m, 1H), 2.68 (m, 2H), 2.15-1.73 (m, 4H), 1.13 (d, 4H).

### EXAMPLE 350

### 5-amino-1-cyclopropyl-8-fluoro-7-(7-hydroxy-4,5,6,7-tetrahydro-1-benzothien-2-yl)-4-oxo-1,4-dihydroguinoline-3-carboxylic acid

The desired product was prepared by substituting Example 301 for Example 326F in Example 349. The crude product was purified by C 18 reverse phase flash chromatography eluting with 1:1:0.01 water/acetonitrile/TFA to give the desired product as a brown solid. MS (DCI) m/z 415 (M+H)⁺; ¹H NMR (300 MHz, DMSO-d₆) δ 14.72 (bs, 1H), 8.57 (s, 1H), 7.49 (bs, 2H), 7.28 (s, 1H), 6.95 (d, 1H), 5.50 (bd, 1H), 4.74 (bm, 1H), 4.11 (m, 1H), 2.59 (m, 2H), 2.07-1.62 (m, 4H), 1.12 (m, 4H).

### EXAMPLE 351

### 5-amino-1-cyclopropyl-8-fluoro-7-(4-hydroxy-4,5,6,7-tetrahydro-1-benzothien-2-yl)-4-oxo-1,4-dihydroquinoline-3-carboxylic acid

A solution of Example 300 (337 mg, 0.81 mmol) in 1-methyl-2-pyrrolidinone (3 mL) saturated with gaseous ammonia was heated in a sealed tube at 100° C. for 72 hours, cooled, diluted with water (4 mL) and brought to pH 5 with AcOH. The crude product was collected by filtration, washed with water and dried to give the desired material as a yellow powder (260 mg, 77%). MS (DCI) m/z 415 (M+H)⁺; ¹H NMR (300 MHz, DMSO-d₆) δ 14.73 (bs, 1H), 8.56 (s, 1H), 7.50 (s, 3H), 6.95 (d, 1H), 5.19 (d, 1H), 4.60 (m, 1H), 4.11 (m, 1H), 2.74 (m, 2H), 2.00-1.65 (m, 4H), 1.14-1.05 (m, 4H).

### EXAMPLE 352

### 5-amino-1-cyclopropyl-7-(4-hydroxy-4,5,6,7-tetrahydro-1-benzothien-2-yl)-4-oxo-1,4-dihydroquinoline-3-carboxylic acid

The desired product was prepared by substituting Example 303C for Example 300 in Example 351 and was triturated in 5:1 methanol/hexanes and filtered to give the desired material as a yellow powder. MS (ESI) m/z 397 (M+H)⁺; ¹H NMR (300 MHz, DMSO-d₆) δ 15.05 (s, 1H), 8.55 (s, 1H), 7.72 (bs, 2H), 7.51 (s, 1H), 7.31 (s, 1H), 6.91 (s, 1H), 5.15 (d, 1H), 4.59 (m, 1H), 3.70 (m 1H), 2.73 (m, 2H), 1.93 (m, 2H), 1.70 (m, 2H), 1.28 (m, 2H), 1.14 (m, 2H).

### EXAMPLE 353

### ethyl 5-(acetylamino)-7-bromo-6-chloro-1-cyclopropyl-8-fluoro-4-oxo-1,4-dihydroquinoline-3-carboxylate

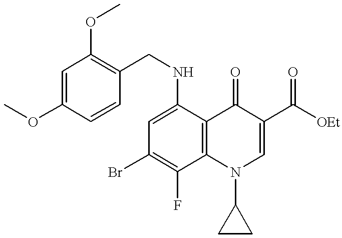

### EXAMPLE 353A

A mixture of 2,4-dimethoxybenzyl amine (18.0 g, 110 mmol), Example 323 (13.7 g, 36.9 mmol) and triethylamine (3.8 g, 37.0 mmol) in trichloroethylene (200 mL) was refluxed for three hours, cooled, washed with water, brine, dried (Na₂SO₄) and concentrated. The crude product was triturated in 2:1 hexane/EtOAc (100 mL) and the desired product was collected by filtration and dried to give a yellow solid (15.6 g, 82%).

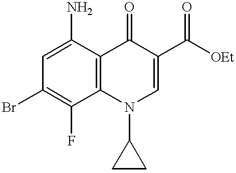

### EXAMPLE 353B

A slurry of Example 353A (15.6 g, 30 mmol) in dichloromethane (30 mL) at 5° C. was treated dropwise with trifluoroacetic acid (20 mL), stirred for 3 hours and concentrated. The residue was azeotroped 2×50 mL with toluene triturated in 1:1 hexane/EtOAc (100 mL) and the yellow solid was collected by filtration and dried (12.0 g, quantitative yield).

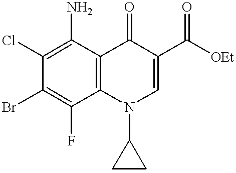

### EXAMPLE 353C

### ethyl 5-amino-7-bromo-6-chloro-1-cyclopropyl-8-fluoro-4-oxo-1,4-dihydroquinoline-3-carboxylate

A slurry of Example 353B (7.36 g, 20 mmol) in dichloromethane (300 mL) at 5° C. was treated dropwise with a solution of 1,3-dichloro-5,5-dimethylhydantoin (Aldrich, 4.3 g, 22 mmol) in dichloromethane (50 mL). The reaction mixture was stirred at ambient temperature for 3 hours, cooled to 5° C., treated slowly with 5% aqueous NaHSO₃ (200 mL) and stirred for 15 minutes. The layers were separated and the dichloromethane layer was washed with water, brine, dried (Na₂SO₄) and concentrated. The crude product was triturated with 2:1 hexane/EtOAc (200 mL) and the orange solid collected by filtration and dried (4.45 g, 55%).

### EXAMPLE 353D

### ethyl 5-(acetylamino)-7-bromo-6-chloro-1-cyclopropyl-8-fluoro-4-oxo-1,4-dihydroquinoline-3-carboxylate

The desired product was prepared by substituting Example 353C for ethyl 5-amino-1-cyclopropyl-6,7,8-trifluoro-4-oxo-1,4-dihydro-quinoline-3-carboxylate in Example 332A and was purified by flash chromatography on silica eluting with first 1% and then 5% MeOH in dichloromethane. MS (DCI) m/z 445 (M+H)⁺; ¹H NMR (300 MHz, CDCl₃) δ 11.37 (s, 1H), 8.53 (s, 1H), 4.38 (q, 2H), 3.95 (m, 1H), 2.25 (s, 3H), 1.39 (t, 3H), 1.27 (d, 2H), 1.06 (dd, 2H).

### EXAMPLE 354

### 5-amino-7-(7-amino-4,5,6,7-tetrahydro-1-benzothien-2-yl)-6-chloro-1-cyclopropyl-8-fluoro-4-oxo-1,4-dihydroquinoline-3-carboxylic acid hydrochloride

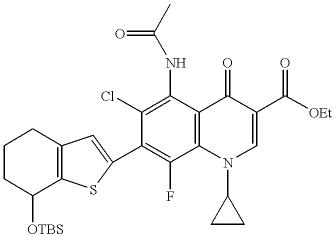

### EXAMPLE 354A

The desired product was prepared by substituting Example 353D and Example 41B for Example 266C and Example 38B, respectively, in Example 296A and was purified by silica gel chromatography eluting with 2:1 hexane/acetone to provide the desired compound as a white solid.

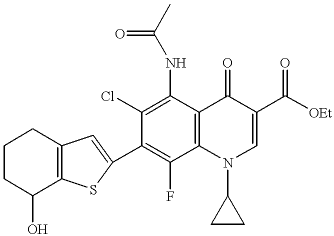

### EXAMPLE 354B

The desired product was prepared by substituting Example 354A for Example 35D in Example 35E and was purified by silica gel chromatography eluting with 97:3 dichloromethane/MeOH.

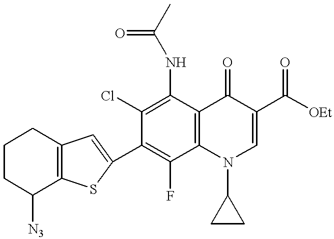

### EXAMPLE 354C

The desired product was prepared by substituting Example 354B for Example 37A in Example 37B using dichloromethane as both the reaction and extraction solvent. The crude product was purified by silica gel chromatography eluting with 2:1 hexane/acetone to provide the desired compound.

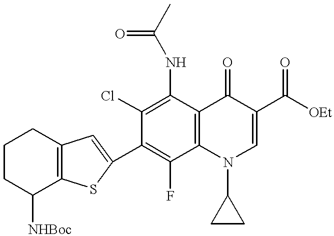

### EXAMPLE 354D

The desired product was prepared by substituting Example 354C for Example 201C in Example 201D and was purified by silica gel chromatography eluting with 1:1 hexane/acetone to provide the desired compound.

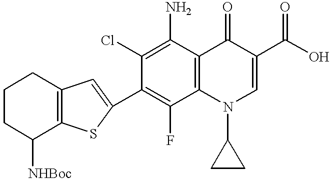

### EXAMPLE 354E

The desired product was prepared as a yellow foam by substituting Example 354D for Example 333-D in Example 333-E and was used without purification.

### EXAMPLE 354F

### 5-amino-7-(7-amino-4,5,6,7-tetrahydro-1-benzothien-2-yl)-6-chloro-1-cyclopropyl-8-fluoro-4-oxo-1,4-dihydroquinoline-3-carboxylic acid hydrochloride

The desired product was prepared as a yellow solid by substituting Example 354E for Example 40B in Example 40C. MS (DCI) m/z 448 (M+H)⁺; ¹H NMR (300 MHz, DMSO-d₆) δ 14.30 (bs, 1H), 8.66 (s, 1H), 8.54 (bs, 3H), 7.85 (bs, 2H), 7.07 (s, 1H), 4.63 (m, 1H), 4.05 (m, 1H), 2.68 (m, 2H), 2.18-1.73 (m, 4H), 1.11 (m, 4H).

### EXAMPLE 355

### 5-amino-6-chloro-1-cyclopropyl-8-fluoro-7-(7-hydroxy-4,5,6,7-tetrahydro-1-benzothien-2-yl)-4-oxo-1,4-dihydroquinoline-3-carboxylic acid

The desired product was prepared by substituting Example 354B for Example 333D in Example 333E. The reaction mixture was adjusted to pH 5 with 1M H₃PO₄ giving a yellow solid which was collected by filtration and dried. MS (DCI) m/z 449 (M+H)⁺; ¹H NMR (300 MHz, DMSO-d₆) δ 8.63 (s, 1H), 7.95-7.65 (bs, 211), 6.95 (s, 111), 5.50 (d, ¹H), 4.76 (m, 111), 4.05 (m, 1H), 2.61 (m, 211), 2.07-1.68 (m, 411), 1.10 (m, 411).

### EXAMPLE 356

### 5-amino-6-chloro-1-cyclopropyl-8-fluoro-7-(4-hydroxy-4,5,6,7-tetrahydro-1-benzothien-2-yl)-4-oxo-1,4-dihydroquinoline-3-carboxylic acid

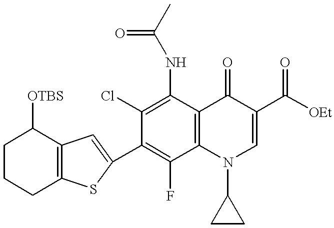

### EXAMPLE 356A

The desired product was prepared by substituting Example 353D for Example 266C in Example 296A and was purified by silica gel chromatography eluting with 2:1 hexane/acetone to provide the desired compound as a yellow solid.

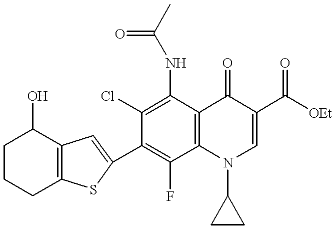

### EXAMPLE 356B

The desired product was prepared by substituting Example 356A for Example 35D in Example 35E and was purified by silica gel chromatography eluting with 97:3 dichloromethane/MeOH to provide the desired compound.

### EXAMPLE 356C

### 5-amino-6-chloro-1-cyclopropyl-8-fluoro-7-(4-hydroxy-4,5,6,7-tetrahydro-1-benzothien-2-yl)-4-oxo-1,4-dihydroquinoline-3-carboxylic acid

The desired product was prepared by substituting Example 356B for Example 333D in Example 333E and was triturated in 9:1 hexane/diethyl ether and the yellow solid collected by filtration and dried. MS (APCI) m/z 449 (M+H)⁺; ¹H NMR (300 MHz, DMSO-d₆) δ 14.37 (s, 1H), 8.64 (s, 1H), 7.95-7.56 (bs, 2H), 7.15 (s, 1H), 5.17 (d, 1H), 4.63 (m, 11H), 4.06 (m, 1H), 2.76 (m, 2H), 2.05-1.66 (m, 4H), 1.10 (m, 4H).

### EXAMPLE 357

### ethyl 7-bromo-8-chloro-1-cyclopropyl-6-fluoro-4-oxo-1,4-dihydroquinoline-3-carboxylate

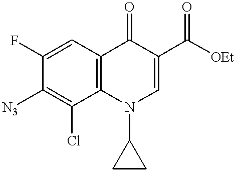

### EXAMPLE 357A

The desired product was prepared as an off-white solid by substituting ethyl 1-cyclopropyl-8-chloro-6,7-difluoro-4-oxo-1,4-dihydro-3-quinoline-3-carboxylate (prepared by the method disclosed in U.S. Pat. No. 4,885,368A) for ethyl 1-cyclopropyl-6,7-difluoro-4-oxo-1,4-dihydro-3-quinoline-3-carboxylate in Example 266A.

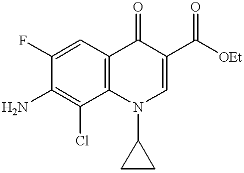

### EXAMPLE 357B

The desired product was prepared by substituting Example 357A and sulfided 5% Pt/C for Example 266A and 10% Pd/C, respectively, in Example 266B and was triturated in a minimal amount of dichloromethane and collected by filtration.

### EXAMPLE 357C

### ethyl 7-bromo-8-chloro-1-cyclopropyl-6-fluoro-4-oxo-1,4-dihydroquinoline-3-carboxylate

The desired product was prepared by substituting Example 357B for Example 266B in Example 266C and was used without purification. MS (DCI/NH₃) m/z 390 (M+H)⁺; ¹H NMR (300 MHz, CDCl₃) δ 8.69 (s, 1H), 8.15 (d, 1H), 4.40 (q, 2H), 4.22 (m, 1H), 1.41 (t, 3H), 1.27 (m, 2H), 0.96 (m, 2H).

### EXAMPLE 358

### 8-chloro-1-cyclopropyl-6-fluoro-7-(7-hydroxy-4,5,6,7-tetrahydro-1-benzothien-2-yl)-4-oxo-1,4-dihydroquinoline-3-carboxylic acid

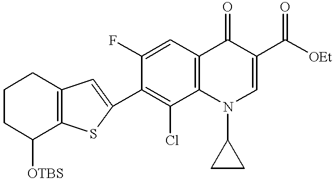

### EXAMPLE 358A

The desired product was prepared by substituting Example 357C and Example 41B for Example 266C and Example 38B, respectively, in Example 296A and was purified by silica gel chromatography eluting with 2:1 hexane/acetone to provide the desired compound.

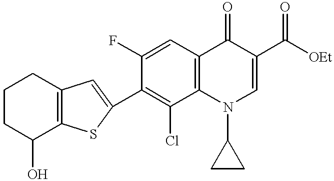

### EXAMPLE 358B

The desired product was prepared by substituting Example 358A for Example 35D in Example 35E and was used without purification.

### EXAMPLE 358C

### 8-chloro-1-cyclopropyl-6-fluoro-7-(7-hydroxy-4,5,6,7-tetrahydro-1-benzothien-2-yl)-4-oxo-1,4-dihydroquinoline-3-carboxylic acid

The desired product was prepared by substituting Example 358B for Example 296B in Example 296C. The reaction mixture was adjusted to pH 5 with 1M H₃PO₄ giving a yellow solid which was collected by filtration and dried. MS (ESI) m/z 434 (M+H)⁺; ¹H NMR (300 MHz, DMSO-d₆) δ 14.24 (s, 1H), 8.91 (s, 1H), 8.10 (d, 1H), 7.21 (s, 1H), 5.19 (d, 1H), 4.64 (m, 1H), 4.42 (m, 1H), 2.79 (m, 2H), 1.97 (m, 2H), 1.74 (m, 2H), 1.22 (m, 2H), 1.09 (m, 2H).

### EXAMPLE 359

### 8-chloro-1-cyclopropyl-6-fluoro-7-(4-(methylamino-4,5,6,7-tetrahydro-1-benzothien-2-yl)-4-oxo-1,4-dihydroquinoline-3-carboxylic acid hydrochloride

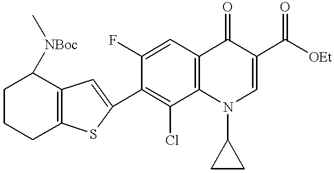

### EXAMPLE 359A

The desired product was prepared by substituting Example 357C and Example 210B for Example 266C and Example 38B, respectively, in Example 296A and was purified by silica gel chromatography eluting with 2:1 hexane/acetone to provide the desired compound as an off-white solid.

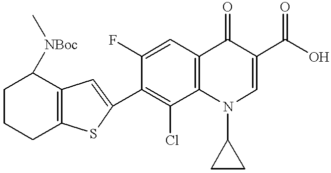

### EXAMPLE 359B

The desired product was prepared by substituting Example 359A for Example 296B in Example 296C.

### EXAMPLE 359C

### 8-chloro-1-cyclopropyl-6-fluoro-7-(4-(methylamino)-4,5,6,7-tetrahydro-1-benzothien-2-yl)-4-oxo-1,4-dihydroquinoline-3-carboxylic acid hydrochloride The desired product was prepared by substituting Example 359B for Example 40B in Example 40C. MS (ESI) m/z 447 (M+H)⁺; ¹H NMR (300 MHz, DMSO-d₆) δ 15.05 (s, 1H), 9.16 (bs, 2H), 8.93 (s, 1H), 8.14 (d, 1H), 7.56 (s, 1H), 4.43 (m, 1H), 3.47 (m, 2H), 2.91 (m, 1H), 2.61 (t, 3H), 2.07 (m, 3H), 1.88 (m, 1H), 1.21 (m, 2H), 1.10 (m, 2H).

### EXAMPLE 360

### 8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid

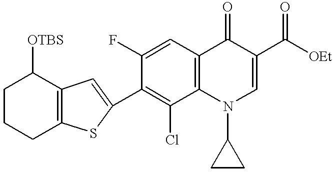

### EXAMPLE 360A

The desired product was prepared by substituting Example 357C for Example 266C in Example 296A and was purified by silica gel chromatography eluting with 2:1 hexane/acetone to provide the desired compound as an off-white solid.

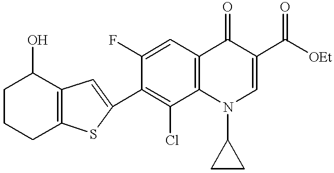

### EXAMPLE 360B

The desired product was prepared by substituting Example 360A for Example 35D in Example 35E and was purified by silica gel chromatography eluting with 2:1 hexane/acetone to provide the desired compound.

### EXAMPLE 360C 8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid

The desired product was prepared by substituting Example 360B for Example 296B in Example 296C. MS (ESI) m/z 434 (M+H)⁺¹H NMR (300 MHz, DMSO-d₆) δ 14.24 (s, 1H), 8.91 (s, 1H), 8.11 (d, 1H), 7.01 (s, 1H), 5.54 (d, 1H), 4.78 (m, 1H), 4.42 (m, 1H), 2.62 (br s, 2H), 1.98 (m, 2H), 1.73 (m, 2H), 1.22 (m, 2H), 1.09 (m, 2H).

### EXAMPLE 361

### 8-chloro-1-cyclopropyl-7-(4-(methylamino)-4,5,6,7-tetrahydro-1-benzothien-2-yl)-4-oxo-1,4-dihydroquinoline-3-carboxylic acid hydrochloride

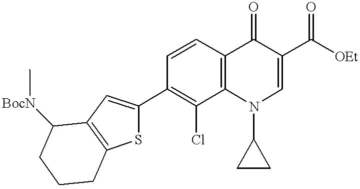

### EXAMPLE 361A

The desired product was prepared by substituting ethyl 7-bromo-8-chloro-1-cyclopropyl-4-oxo-1,4-dihydroquinoline-3-carboxylate (prepared by the method disclosed in U.S. Pat. No. 6,025,370), Example 210B, and a reaction time of 6 h for ethyl 7-bromo-1-cyclopropyl-4-oxo-1,4-dihydro-3-quinolone-3-carboxylate, Example 1D, and 24 h, respectively, in Example 1E and was purified by silica gel chromatography eluting with a gradient of 2:1 to 1:1 hexane/acetone to yield an off-white solid.

### EXAMPLE 361B

### 8-chloro-1-cyclopropyl-7-(4-(methylamino)-4,5,6,7-tetrahydro-1-benzothien-2-yl)-4-oxo-1,4-dihydroquinoline-3-carboxylic acid hydrochloride

The desired product was prepared by substituting Example 361A for Example 48C and tetrahydrofuran for ethanol in Example 48D to yield an off-white solid. MS (DCI/NH₃) m/z 429 (M+H)⁺; ¹H NMR (300 MHz, DMSO-d₆) δ 9.05 (br s, 1H), 8.93 (s, 1H), 8.35 (d, 1H), 7.79 (m, 2H), 4.46 (m, 2H), 2.90(m, 2H), 2.65 (m, 3H), 2.09-1.91 (m, 4H), 1.24-1.03 (m, 4H).

### EXAMPLE 362

### 7-(7-amino-4,5,6,7-tetrahydro-1-benzothien-2-yl)-1-cyclopropyl-8-(difluoromethoxy)-6-fluoro-4-oxo-1,4-dihydroquinoline-3-carboxylic acid hydrochloride

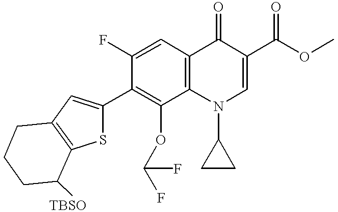

### EXAMPLE 362A

The desired product was prepared by substituting ethyl 7-bromo-1-cyclopropyl-8-difluoromethoxy-6-fluoro-4-oxo-1,4-dihydroquinoline-3-carboxylate (prepared by the method disclosed in U.S. Pat. No. 5,935,952) and Example 41B for ethyl 7-bromo-1-cyclopropyl-4-oxo-1,4-dihydroquinoline-3-carboxylate, and Example 1D in Example 1E. The crude product was substituted for Example 38C in Example 39A and was purified by silica gel chromatography eluting with a gradient of 2:1 to 1:1 hexane/acetone to yield a pale yellow solid.

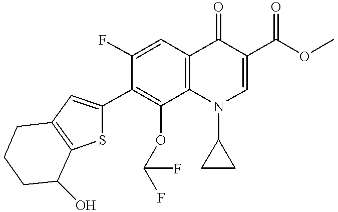

### EXAMPLE 362B

The desired product was prepared by substituting Example 362A for Example 35D in Example 35E to yield a pale yellow solid.

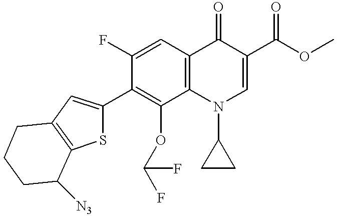

### EXAMPLE 362C

The desired product was prepared by substituting Example 362B for Example 37A in Example 37B and was purified by silica gel chromatography eluting with a gradient of 4:1 to 1:1 hexane/acetone to yield an off-white solid.

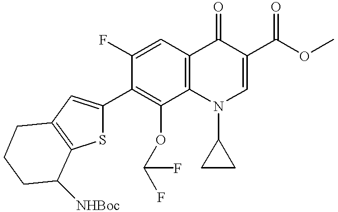

### EXAMPLE 362D

The desired product was prepared by substituting Example 362C for Example 201C in Example 201B and was purified by silica gel chromatography eluting with a gradient of 4:1 to 1:1 hexane/acetone to yield an off-white solid.

### EXAMPLE 362E

### 7-(7-amino-4,5,6,7-tetrahydro-1-benzothien-2-yl)-1-cyclopropyl-8-(difluoromethoxy)-6-fluoro-4-oxo-1,4-dihydroquinoline-3-carboxylic acid hydrochloride

The desired product was prepared by substituting Example 362D for Example 48C and tetrahydrofuran for ethanol in Example 48D to yield an off-white solid. MS (DCI/NH₃) m/z 465 (M+H)⁺; ¹H NMR (300 MHz, DMSO-d₆) δ 8.89 (s, 1H), 8.53 (br s, 2H), 8.15 (d, 1H), 7.35 (s, 1H), 6.91 (t, 1H), 4.64 (m, 1H), 4.12 (m, 1H), 2.72-2.67 (m, 2H), 2.15-1.70 (m, 4H), 1.19-1.06 (m, 4H).

### EXAMPLE 363

### 1-cyclopropyl-8-(difluoromethoxy)-6-fluoro-7-(7-hydroxy-6,6-dimethyl-4,5,6,7-tetrahydro-1-benzothien-2-yl)-4-oxo-1,4-dihydroquinoline-3-carboxylic acid

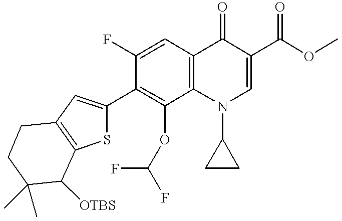

### EXAMPLE 363A

The desired product was prepared by substituting ethyl 7-bromo-1-cyclopropyl-8-difluoromethoxy-6-fluoro-4-oxo-1,4-dihydroquinoline-3-carboxylate (prepared by the method disclosed in U.S. Pat. No. 5,935,952) and Example 241C for ethyl 7-bromo-1-cyclopropyl-4-oxo-1,4-dihydroquinoline-3-carboxylate, and Example 1D in Example 1E. The crude above product was substituted for Example 38C in Example 39A and was purified by silica gel chromatography eluting with a gradient of 2:1 to 1:1 hexane/acetone to yield a pale yellow solid.

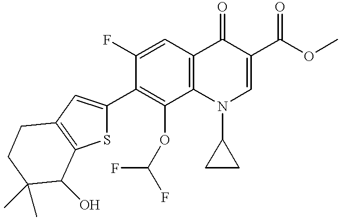

### EXAMPLE 363B

The desired product was prepared by substituting Example 363A for Example 35D in Example 35E to yield a pale yellow solid.

### EXAMPLE 363C

### 1-cyclpropyl-8-(difluoromethoxy)-6-fluoro-7-(7-hydroxy-6,6-dimthethyl-4,5,6,7-tetrahydro-1-benzothien-2-yl)-4-oxo-1,4-dihydroquinoline-3-carboxylic acid

The desired product was prepared by substituting Example 363B for Example 2A and a reaction time of 2.5 h for an overnight reaction time in Example 2B to yield a yellow solid. MS (DCI/NH₃) m/z 494 (M+H)⁺; ¹H NMR (300 MHz, CDCl₃) δ 14.23 (s, 1H), 8.94 (s, 1H), 8.19 (d, 1H), 7.25 (s, 1H), 6.14 (t, 1H), 4.54 (m, 1H), 4.20 (m, 1H), 2.69 (m, 2H), 1.84-0.98 (m, 12H).

### EXAMPLE 364

### ethyl 7-bromo-1-cyclopropyl-6-fluoro-5,8-dimethyl-4-oxo-1,4-dihydroquinoline-3-carboxylate

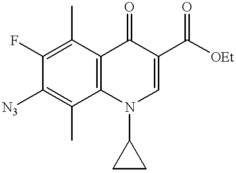

### EXAMPLE 364A

The desired product was prepared by substituting ethyl 1-cyclopropyl-6-fluoro-5,8-dimethyl-4-oxo-1,4-dihydroquinoline-3-carboxylate (prepared by the method of Hagen, S.E., Domagala, J. M. J. Heterocycl. Chem. 1990, 2 7, 1609-1616) and a reaction time of 3 days at 95° C. for ethyl 1-cyclopropyl-6,7-difluoro-4-oxo-1,4-dihydroquinoline-3-carboxylate for 4 h at 65° C., respectively, in Example 266A to yield an orange solid.

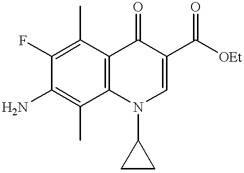

### EXAMPLE 364B

The desired product was prepared by substituting Example 364A for Example 266A in Example 266B to yield an tan solid.

### EXAMPLE 364C

### ethyl 7-bromo-1-cyclopropyl-6-fluoro-5,8-dimethyl-4-oxo-1,4-dihydroquinoline-3-carboxylate

The desired product was prepared by substituting 364Bfor Example 266B in Example 266C to yield an off-white solid. MS (DCI/NH₃) m/z 382 (M+H)⁺; ¹H NMR (300 MHz, DMSO-d₆) δ 8.52 (s, 1H), 4.15 (q, 2H), 4.11 (m, 1H), 2.78 (s, 3H), 2.67 (d, 3H), 1.27 (t, 3H), 1.11-1.05 (m, 2H), 0.71-0.65 (m, 2H).

### EXAMPLE 365

### ethyl 5-(acetylamino)-7-bromo-1-cyclopropyl-6-fluoro-8-methyl-4-oxo-1,4-dihydroquinoline-3-carboxylate

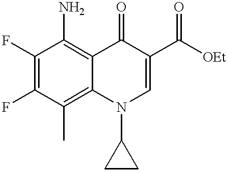

### EXAMPLE 365A

A solution of ethyl 1-cyclopropyl-6,7-difluoro-1,4-dihydro-8-methyl-5-nitro-4-oxoquinoline-3-carboxylate (4.0 g, 11.35 mmol; obtained by the method of Yoshida, et al.; Chem. Pharm. Bull. 1996, 44,1074) in 500 mL DMF was degassed and treated with 10% palladium on carbon (500 mg) followed by hydrogenation at one atmosphere for 6 h. The solution was filtered through celite, washing the filter cake with DMF. The filtrate was concentrated by short path high vacuum distillation of the DMF, with a final distillation pot temperature of 50° C. The solid residue was triturated with ether, collected by filtration and washed with ether to provide the desired product (2.68 g, 73%) as a gray solid.

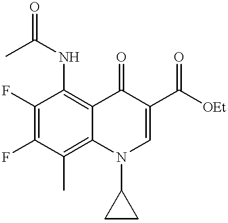

### EXAMPLE 365B

The desired product was prepared by substituting Example 365A for ethyl 5-amino-1-cyclopropyl-6,7,8-trifluoro-4-oxo-1,4-dihydro-quinoline-3-carboxylate as in Example 332A to yield a gray solid.

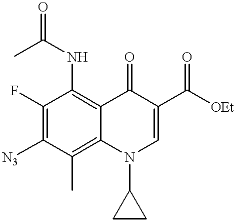

### EXAMPLE 365C

The desired product was prepared by substituting Example 365B for ethyl 5-(acetylamino)-1-cyclopropyl-6,7,8-trifluoro-4-oxo-1,4-dihydro-quinoline-3-carboxylate in Example 332B to yield a gray solid.

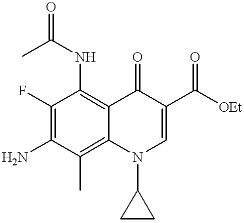

### EXAMPLE 365D

The desired product was prepared by substituting Example 365C for 5-(acetylamino)-7-azido-1-cyclopropyl-6,8-difluoro-4-oxo-1,4-dihydro-quinoline-3-carboxylate in Example 332C to yield a gray-brown solid.

### EXAMPLE 365E

### ethyl 5-(acetylamino)-7-bromo-1-cyclopropyl-6-fluoro-8-methyl-4-oxo-1,4-dihydroquinoline-3-carboxylate

The desired product was prepared by substituting Example 365D for 7-amino-5-(acetylamino)-1-cyclopropyl-6,8-difluoro-4-oxo-1,4-dihydro-quinoline-3-carboxylate in Example 332E to yield a gray solid. MS (ESI) m/z 425,427 (M+H)⁺; ¹H NMR (DMSO-d₆, 300 MHz) δ 10.96 (s, 1 H), 8.60 (s, 1 H), 4.23 (q, 2 H), 4.22 (m, 1 H), 2.82 (s, 3 H), 2.13 (s, 3 H), 1.28 (t, 3 H), 1.12 (m, 2 H), 0.76 (m, 2 H).

### EXAMPLE 366

### 1-cyclopropyl-6-fluoro-7-(4-hydroxy-4,5,6,7-tetrahydro-1-benzothien-2-yl)-5-methyl-4-oxo-1,4-dihydro-1,8-naphthyridine-3-carboxylic acid

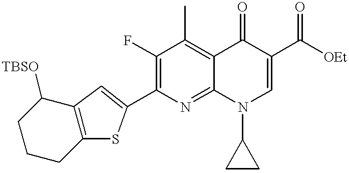

### EXAMPLE 366A

The desired product was prepared by substituting ethyl 7-chloro-1-cyclopropyl-6-fluoro-5-methyl-4-oxo-1,4-dihydro-1,3-napthyridine-3-carboxylate (prepared by the method of Bouzard, et al.; J. Med. Chem. 1992, 35, 518-525) for Example 266C in Example 296A.

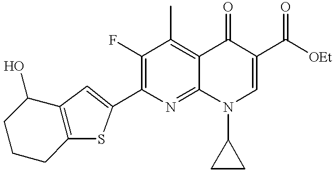

### EXAMPLE 366B

The desired product was prepared by substituting Example 366A for Example 35D in Example 35E to afford the desired compound as a light brown solid.

### EXAMPLE 366C

### 1-cyclopropyl-6-fluoro-7-(4-hydroxy-4,5,6,7-tetrahydro-1-benzothien-2-yl)-5-methyl-4-oxo-1,4-dihydro-1,8-naphthyridine-3-carboxylic acid

A mixture of Example 366B (160 mg, 0.36 mmol) in methanol (1.0 mL), water (1.7 mL) and 2 N NaOH solution (1.7 mL, 3.4 mmol) was warmed at 100° C. for 3 h. The mixture was allowed to cool to ambient temperature and was adjusted to pH 2 by addition of 1 N HCl solution and was extracted with dichloromethane. The organic phase was dried (Na2SO4) and concentrated and the crude solid purified by trituration with ether to provide the desired compound (122 mg, 81%) as a light brown solid. MS (ESI) m/z 415 (M+H)⁺; ¹H NMR (CD₂Cl₂, 300 MHz) δ 14.77 (s, 1H), 8.78 (s, 1H), 7.92 (d, 1H), 4.76 (m, 1H), 3.71 (m, 1H), 2.89 (d, 3H), 2.85 (m, 2H), 2.01 (m, 2H), 1.82 (m, 2H), 1.33 (m, 2H), 1.05 (m, 2H).

### EXAMPLE 367

### 7-(4-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid hydrochloride

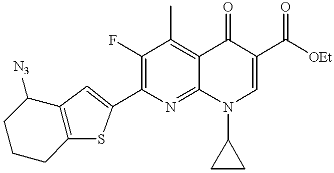

### EXAMPLE 367A

The desired compound was prepared by substituting Example 366B for Example 37A in Example 37B.

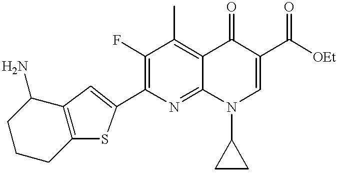

### EXAMPLE 367B

A solution of Example 367A (314 mg, 0.67 mmol) in 12 mL of 1:1:1 methanol-ethyl acetate-dichloromethane was treated with 80 mg of 10% palladium on carbon and 1 atm hydrogen gas for 5 h. The mixture was filtered through celite, washing the filter cake with methanol. The filtrate was concentrated and the residue triturated with ether, collected by filtration and washed with ether to provide the desired product (225 mg, 76%) as a light beige solid.

### EXAMPLE 367C

### 7-(4-amino-4,5,6,7-tetrahydro-1-benzothien-2-yl)-1-cyclopropyl-6-fluoro-5-methyl-4-oxo-1,4-dihydro-1,8-naphthyrddine-3-carboxylic acid hydrochloride

A solution of Example 367B (60 mg, 0.135 mmol) in absolute ethanol (6 mL) was treated with 1 N HCl solution (6 mL) and was heated at 85° C. for 2 h. The reaction mixture was allowed to cool, was diluted with ethanol and concentrated. The solid residue was triturated with ether and collected by filtration to provide the desired compound (47 mg, 78%) as a beige solid. MS (DCI) m/z 414 (M+H)⁺.

### EXAMPLE 368

### 7-(7-amino-4,5,6,7-tetrahydro-1-benzothien-2-yl)-1-cyclopropyl-6-fluoro-8-methyl-4-oxo-1,4-dihydroquinoline-3-carboxylic acid hydrochloride

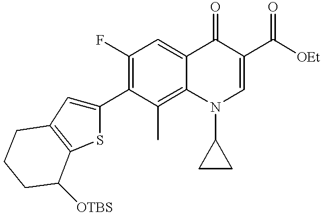

### EXAMPLE 368A

The desired product was prepared by substituting Example 41B and Example 331C for Example 38B and Example 266C in Example 296A.

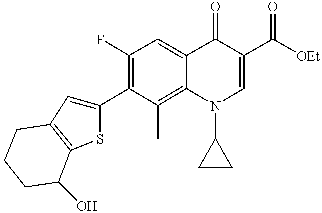

### EXAMPLE 368B

The desired product was prepared by substituting Example 368A or Example 35D in Example 35E and was purified by trituration with 5% acetone in hexanes.

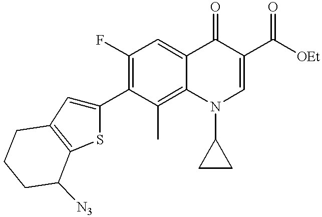

### EXAMPLE 368C

The desired product was prepared by substituting Example 368B for Example 321B in Example 321C.

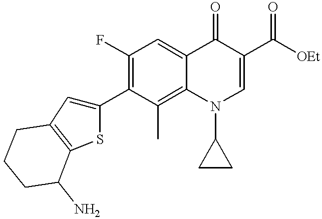

### EXAMPLE 368D

The desired product was prepared by substituting Example 368C for 321C in Example 321D.

### EXAMPLE 368E

### 7-(7-amino-4,5,6,7-tetrahydro-1-benzothien-2-yl)-1-cyclopropyl-6-fluoro-8-methyl-4-oxo-1,4-dihydroquinoline-3-carboxylic acid hydrochloride

The desired product was prepared by substituting Example 368D for Example 48C in Example 48D. MS (DCI/NH₃) m/z 413 (M+H)⁺; ¹H NMR (DMSO-d₆, 300 MHz) δ 8.91 (s, 1H), 8.61 (br s, 3H), 7.97 (d, 1H), 7.09 (s, 1H), 4.62 (m, 1H), 4.39 (m, 1H), 2.75 (s, 3H), 2.71 (m, 2H), 1.80-2.20 (m, 4H), 1.03-1.29 (m, 4H).

### EXAMPLE 369

### 1-cyclopropyl-6-fluoro-7-(7-hydroxy-4,5,6,7-tetrahydro-1-benzothien-2-yl)-8-methyl-4-oxo-1,4-dihydroquinoline-3-carboxylic acid

The desired product was prepared by substituting Example 368B for Example 2A in Example 2B. MS (DCI/NH₃) m/z 414 (M+H)⁺; ¹H NMR (DMSO-d₆, 300 MHz) δ 14.63 (s, 1H), 8.89 (s, 1H), 7.94 (d, 1H), 6.95 (s, 1H), 5.51 (d, 1H), 4.77 (m, 1H), 4.38 (m, 1H), 2.76 (s, 3H), 2.62 (br s, 2H), 1.70-2.07 (m, 4H), 1.02-1.27 (m, 4H).

### EXAMPLE 370

### 1-cyclopropyl-6-fluoro-7-(7-hydroxy-4,5,6,7-tetrahydro-1-benzothien-2-yl)-5-methyl-4-oxo-1,4-dihydro-1,8-naphthyridine-3-carboxylic acid

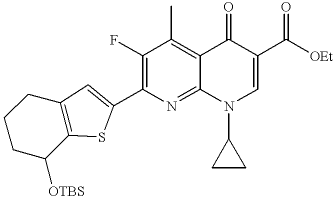

### EXAMPLE 370A

The desired product was prepared by substituting Example 41B and ethyl 7-chloro-1-cyclopropyl-6-fluoro-5-methyl-4-oxo-1,4-dihydro-1,8-napthyridine-3-carboxylate (prepared by the method of Bouzard, D.; Cesare, P. Di; Essiz, M.; Jacquet, J. P.; Ledoussal, B.; et al.; J. Med. Chem. 1992, 35, 518-525) for Example 38B and Example 266C, respectively in Example 296A.

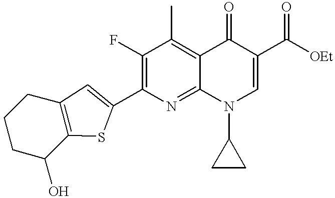

### EXAMPLE 370B

The desired product was prepared by substituting Example 370A for Example 35D in Example 35E to yield a light beige solid.

### EXAMPLE 370C

### 1-cyclopropyl-6-fluoro-7-(7-hydroxy-4,5,6,7-tetrahydro-1-benzothien-2-yl)-5-methyl-4-oxo-1,4-dihydro-1,8-naphthyridine-3-carboxylic acid

The desired product was prepared by substituting Example 370B for Example 366B in Example 366C to yield a gray solid. MS (DCI) m/z 415 (M+H)⁺.

### EXAMPLE 371

### 7-(7-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid hydrochloride

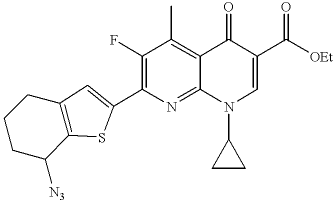

### EXAMPLE 371A

The desired product was prepared by substituting Example 370B for Example 37A in Example 37B to yield an orange solid.

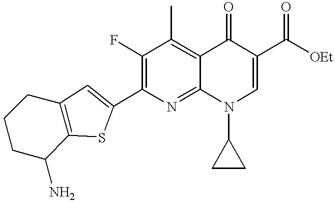

### EXAMPLE 371B

The desired product was prepared by substituting Example 370A for Example 367A in Example 367B to yield a red brown solid.

### EXAMPLE 371C

### 7-(7-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid hydrochloride

The desired product was prepared by substituting Example 371B for Example 367B in 367C to give the desired compound as a yellow solid. MS (DCI) m/z 414 (M+H)⁺; ¹H NMR (DMSO-d₆, 300 MHz) δ 14.81 (m, 1H), 8.79 (s, 1H), 8.62 (br s, 3H), 7.81 (d, 1H), 4.63 (br m, 1H), 3.78 (m, 1H), 2.89 (d, 3H), 2.70 (m, 2 H), 2.14 (m, 1H), 1.98 (m, 2H), 1.82 (m, 1 H), 1.33 (m,2H), 1.16 (m,2H).

### EXAMPLE 372

### 7-(7-amino-6,6-dimethyl-4,5,6,7-tetrahydro-1-benzothien-2-yl)-1-cyclopropyl-6-fluoro-8-methyl-4-oxo-1,4-dihydroquinoline-3-carboxylic acid hydrochloride

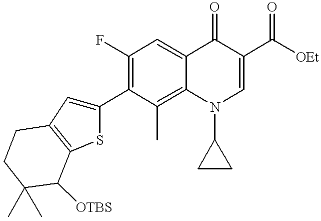

### EXAMPLE 372A

The desired product was prepared by substituting Example 241C and Example 331C for Example 38B and Example 266C, respectively, in Example 296A and was purified by silica gel chromatography eluting with 33 % acetone in hexanes.

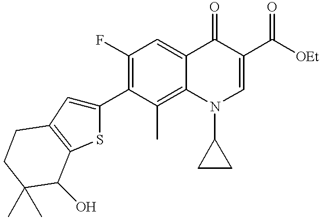

### EXAMPLE 372B

The desired product was prepared by substituting Example 372A for Example 35D in Example 35E and was purified by trituration in 5 % acetone in hexanes.

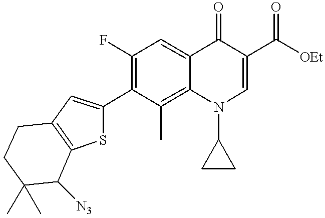

### EXAMPLE 372C

A solution of Example 372B (0.71 g, 1.51 mmol) in dichloromethane (20 mL) was cooled to 0° C., treated with DPPA (0.98 mL, 4.55 mmol) followed by DBU (0.70 mL, 4.68 mmol), allowed to warm to ambient temperature and stirred for 48 h. The reaction mixture was washed with water, 1N HCl, brine, dried (Na₂SO₄), and concentrated. The crude residue was purified by chromatography on silica gel eluting with a gradient of 0% to 25% acetone in hexanes to provide the desired product as an off-white solid (0.360 g, 48.3%).

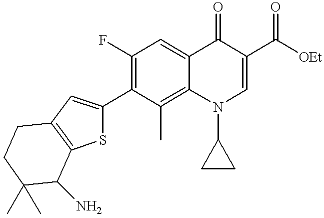

### EXAMPLE 372D

The desired product was prepared by substituting Example 372C for 321C in Example 321D.

### EXAMPLE 372E

### 7-(7-amino-6,6-dimethyl-4,5,6,7-tetrahydro-1-benzothien-2-yl)-1-cyclopropyl-6-fluoro-8-methyl-4-oxo-1,4-dihydroquinoline-3 -carboxylic acid hydrochloride

The desired product was prepared by substituting Example 372D for Example 48C in Example 48D. MS (DCI/NH₃) m/z 441 (M+H)⁺; ¹H NMR (DMSO-d₆, 300 MHz) δ 8.91 (s, 1H), 8.49 (br s, 3H), 7.97 (d, 1H), 7.08 (s, 1H), 4.41 (m, 1H), 4.39 (m, 1H), 2.75 (s, 3H), 2.63-2.75 (m, 2H), 1.60-1.93 (m, 2H), 0.94-1.31 (m, 4H), 1.09 (br s, 6H).

### EXAMPLE 373

### 7-(7-amino-6,6-dimethyl-4,5,6,7-tetrahydro-1-benzothien-2-yl)-1-cyclopropyl-6,8-difluoro-4-oxo-1,4-dihydroquinoline-3-carboxylic acid hydrochloride

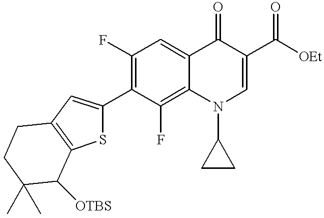

### EXAMPLE 373A

The desired product was prepared by substituting Example 241 C and Example 324 for Example 38B and Example 266 in Example 296A and was purified by silica gel chromatography eluting with 33 % acetone in hexanes.

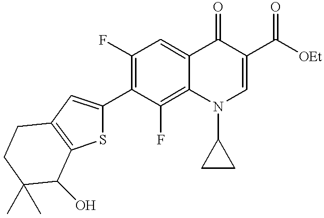

### EXAMPLE 373B

The desired product was prepared by substituting Example 373A for Example 35D in Example 35E and was purified by trituration in 5% acetone in hexanes.

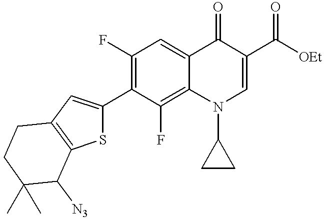

### EXAMPLE 373C

The desired product was prepared by substituting Example 373B for Example 372B in Example 372C.

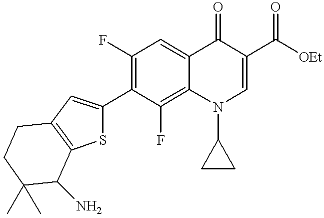

### EXAMPLE 373D

The desired product was prepared by substituting Example 373C for 321C in Example 321D.

### EXAMPLE 373E

### 7-(7-amino-6,6-dimethyl-4,5,6,7-tetrahydro-1-benzothien-2-yl)-1-cyclopropyl-6,8-difluoro-4-oxo-1,4-dihydroquinoline-3 -carboxylic acid hydrochloride

The desired product was prepared by substituting Example 373D for Example 48C in Example 48D. MS (DCI/NH₃) m/z 445 (M+H)⁺; ¹H NMR (300 MHz, DMSO-d₆) δ 8.97 (s, 1H), 8.05 (dd, 1H), 4.31 (m, 1H), 4.20 (m, 1H), 2.65-2.76 (m, 2H), 1.60-1.92 (m, 2H), 1.10-1.29 (m, 4H), 1.08 (br s, 6H).

### EXAMPLE 374

### ethyl 7-bromo-1-cyclopropyl-6-fluoro-8-methoxy-4-oxo-1,4-dihydroquinoline-3-carboxylate

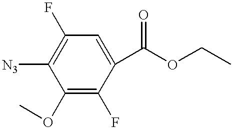

### EXAMPLE 374A

A solution of ethyl 3-methoxy-2,4,5-trifluorobenzoate (79.5 g, 0.339 mol; prepared by the method of Sanchez, Joseph P., Gogliotti, Rocco D., Domagala, John M., Gracheck, Stephen J., Huband, Michael D., et al., J. Med. Chem. 1995, 38,4478-4487.) in DMSO (350 ml) was treated with NaN₃ (50.72 g, 0.781 mol), heated to 55° C. for 24h, allowed to cool and was poured into water. This mixture was extracted with ethyl acetate, the organic phase washed with brine, dried (MgSO₄), treated with activated carbon, filtered and the filtrate concentrated to provide the desired product (70.72 g, 81%) as a light yellow solid.

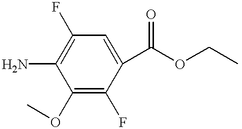

### EXAMPLE 374B

A solution of Example 374A ( 70.7 g, 275mmol) in ethanol (500 mL) was treated with 5% Pt/C ( 3.5 g ) and treated with 50 PSI hydrogen gas in a Parr shaker for 24 h, filtered and the filtrate concentrated to provide the desired product (62.78 g, 99%) as golden colored

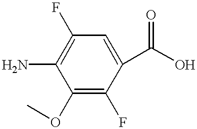

### EXAMPLE 374C

A solution of Example 374B (62.77 g, 0.271mol) in THF ( 1 L) was cooled in an ice-bath at 4° C., treated with LiOH.H2O (13.92 g, 0.331 mol) followed by H₂(250 mL), stirred at this temperature for 1 h then at room temperature for 24 h. The reaction mixture was concentrated and the residue acidified with 10% aqueous HBr to provide the desired product (50.91 g, 92%) as an off white solid.

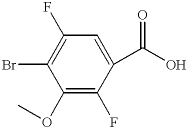

### EXAMPLE 374D

To a solution of Example 374C (8.01 g, 0.039 mol) in 4.8% HBr (200 mL) was added CuBr₂ (49.9,223 mmol) followed by a solution of sodium nitrite (5.18 g, 0.075 mol) in water (55 mL) dropwise. The reaction mixture was stirred at 0° C. for 1 h and then at room temperature for 24 h. The reaction mixture was extracted with ethyl acetate, the organic phase washed with 20% aqueous HBr, and dried (MgSO₄). Concentration gave the desired product as a tan solid (9.83 g, 93%) that was used without further purification.

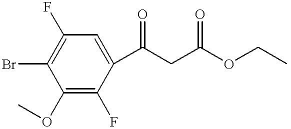

### EXAMPLE 374E

A solution of Example 374D (9.82 g, 0.039 mol) in CH₂Cl₂ (100 ml) was cooled to 0° C., treated dropwise with oxalyl chloride (9.35 g, 0.074 mol), stirred at 0 ° C. for 1 h, at room temperature for 14 h and then concentrated to dryness to provide the acid chloride as an amber oil.

A solution of potassium ethyl malonate (12.93 g, 0.076 mol) in acetonitrile (250 ml) was cooled to 0° C., treated with MgCl₂ (8.94 g, 0.092 mol), Et3N (10.95 ml, 0.148 mol), stirred 3 h and treated dropwise with a solution of the crude acid chloride above in acetonitrile (50 ml). The resulting mixture was stirred at 0 ° C. for 1 h then at room temperature for 14 h. The reaction mixture was acidified with 4N HCl (50 ml), stirred 1 h and partitioned between water and CH₂Cl₂. The aqueous phase was extracted with CH₂Cl₂, the combined organic phases washed with 5% aqueous NaHCO₃, brine and dried (MgSO₄). Concentration gave the desired product (12.08 g, 87%) as an off-white solid.

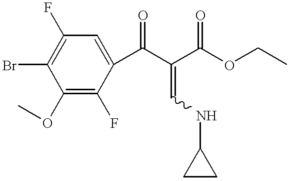

### EXAMPLE 374F

A solution of Example 374E (10.83 g, 0.032 mol) in acetic anhydride (15.0 g, 0.147 mol) was treated with triethylorthoformate (7.27 g, 0.049 mol) and heated to 130° C. for 2 h and was allowed to cool to room temperature. The mixture was concentrated to dryness in vacuo and residual reagents removed by azeotropic distillation with toluene. The resulting amber oil was reconstituted in CH₂Cl₂ (60 ml), cooled to 0° C. and treated dropwise with cyclopropylamine (2.02 g, 0.036 mol), allowed to warm to room temperature and stir for 14 h. The resulting mixture was concentrated to give a crystalline material that was triturated in hexanes and filtered to provide the desired product (12.0 g, 92%) as an off-white solid.

### EXAMPLE 374G

A solution of Example 374F (11.83 g, 0.029 mol) in DMF (150 ml) was treated with K₂CO₃ (12.13 g, 0.088 mol), heated to 100° C. for 1.5 h, allowed to cool to room temperature and poured into water (1 L). The resulting heterogeneous mixture was filtered, the solid washed with water and dried to give the desired product (10.72 g, 95%) as a yellow solid. MS (ESI) m/z 384 (M+H)⁺; ¹H NMR (300 MHz, CDCl₃) δ 8.62 (s, 1H), 8.04 (d, 1H), 4.40 (q, 2H), 3.93 (m, 1H), 3.90 (s, 3H), 1.42 (t, 3H), 1.23 (m, 2H), 1.02 (m, 2H).

Using the schemes and experimentals discussed hereinabove, the following compounds were prepared. The MIC data for the compounds is presented in Table 1 or Table 2.

What is claimed is:

1. A compound selected from the group consisting of compounds of formula (I) 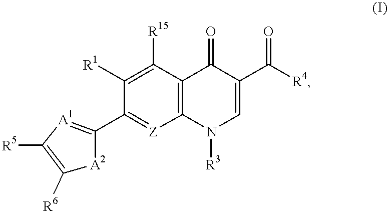
or pharmaceutically acceptable salts or prodrugs thereof, wherein
A¹ is nitrogen or 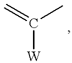
wherein W is selected from the group consisting of
(1) hydrogen and
(2) optionally substituted alkyl;
A is selected from the group consisting of
(1) —S—,
(2) —O—, and
(3) —N(R⁷)—, wherein R⁷ is hydrogen or C₁-C₆ alkyl;
R¹ and R¹⁵ are independently selected from the group consisting of
(1) hydrogen,
(2) optionally substituted alkyl,
(3) halide,
(4) nitro, and
(5) optionally protected amino;
Z is nitrogen or 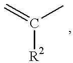
wherein R² is selected from the group consisting of
(1) hydrogen,
(2) optionally substituted alkyl,
(3) halide,
(4) optionally protected hydroxyl,
(5) —OR⁸, and
(6) —S(O)ₙR⁸, wherein n is zero, one, or two, and
wherein R⁸ in (4) and (5) is selected from the group consisting of
(a) C₃-C₆ alkenyl,
(b) C₁-C₆ alkyl, and
(c) C₃-C₆ alkynyl,
wherein (a)-(c) can be optionally substituted with one, two, or three substituents independently selected from the group consisting of
(i) alkoxy,
(ii) aryl,
(iii) C₃-C₆ cycloalkyl,
(iv) azido,
(v) cyano,
(vi) halide,
(vii) optionally protected amino,
(viii) optionally protected carboxyl, and
(ix) optionally protected hydroxyl;
R³ is selected from the group consisting of
(1) C₃-C₆ alkenyl,
(2) C₁-C₆ alkyl,
(3) C₃-C₆ alkynyl,
wherein (1)-(3) can be optionally substituted with one, two, or three substutuents independently selected from the group consisting of
(a) C₁-C₆ alkanoyloxy,
(b) C₁-C₆ alkoxy,
(c) aryl,
(d) azido,
(e) cyano,
(f) C₃-C₆ cycloalkyl,
(g) halide,
(h) optionally protected amino,
(i) optionally protected carboxyl,
(j) optionally protected hydroxyl,
(k) oxo,
(l) C₁-C₆ perfluoroalkoxy,
(m) C₁-C₆ perfluorothioalkoxy, and
(n) thioxo,
(4) aryl,
(5) C₃-C₆ cycloalkyl, and
(6) heterocycle,
wherein (4)-(6) can be optionally substituted with one, two, or three substituents independently selected from the group consisting of
(a) C₁-C₆ alkanoyloxy,
(b) C₁-C₆ alkoxy,
(c) C₂-C₆ alkenyl,
(d) C₁-C₆ alkyl,
(e) C₂-C₆ alkynyl,
(f) aryl,
(g) azido,
(h) cyano,
(i) C₃-C₆ cycloalkyl,
(l) halide,
(k) optionally protected amino
(l) optionally protected carboxyl,
(m) optionally protected hydroxyl,
(n) C₁-C₆ perfluoroalkoxy, and
(o) Cl-C₆ perfluorothioalkoxy;
or
R² and R³ together are selected from the group consisting of 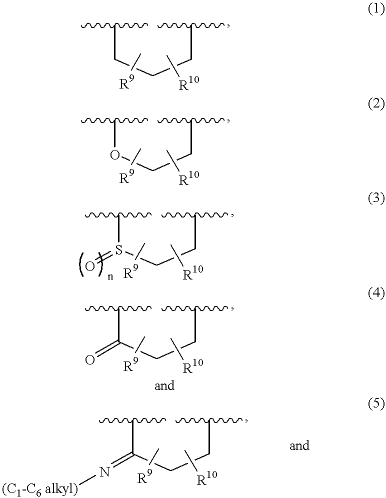
and
wherein one of R⁹ or R¹⁰ in (1)-(5) is hydrogen and the other is selected from the group consisting of
(1) hydrogen,
(2) C₁-C₆ alkyl,
(3) C₁-C₆ haloalkyl, and
(4) optionally protected hydroxyl,
or
wherein R⁹ and R¹⁰ together are alkylidene or C₃-C₆ spiroalkyl;
R⁴ is hydrogen or —OR¹¹, wherein R¹¹ is hydrogen or a carboxyl protecting group; and
R⁵ and R⁶ together are a carbocyclic or a heterocyclic ring, wherein the carbocyclic ring and the heterocyclic ring can be optionally substituted with one, two, or three substituents independently selected from the group consisting of
(1) optionally substituted aryl,
(2) azido,
(3) carboxaldehyde,
(4) cyano,
(5) halide,
(6) nitro,
(7) optionally substituted C₁-C₆ alkyl,
(8) optionally substituted C₃-C₆ alkenyl,
(9) optionally substituted C₃-C₆ alkynyl,
(10) optionally protected amino,
(11) optionally protected hydroxyl,
(12) optionally protected carboxyl,
(13) optionally substituted C₁-C₆ alkanoyloxy,
(14) optionally substituted C₁-C₆ alkoxy,
(15) optionally substituted aryl,
(16) optionally substituted C₃-C₆ cycloalkyl,
(17) optionally substituted heterocycle,
(18) oxo,
(19) C₁-C₆ perfluoroalkoxy,
(20) C₁-C₆ perfluorothioalkoxy,
(21) optionally substituted C₁-C₆ thioalkoxy,
(22) thioxo,
(23) a nitrogen protecting group,
(24) heterocycle,
(25) —C(O)N(R¹²)₂,
(26) —C(O)SR
(27) —N(R¹²)₂,
(28) ═N—,
(29) —OC(O)N(R¹²)₂,
(30) ═N—N(R¹²)₂,
(31) ═N(R¹²)—N(R¹²)₂,
(32) —N(R¹²)—C(═NR¹²)—N(R¹²)₂,
(33) ═NOR¹²,
(34) ═NN(R )C(O)N(R¹²)₂,
(35) —N(R¹²)C(O)N(R¹²)₂,
(36) —C(O)R¹²
(37) —OC(O)R¹²
(38) —N(R¹²)C(O)R¹²,
(39) —N(R¹²)C(O)OR¹²
(40) —N(R¹²)S(O)ₙR¹²,
(41) —OR¹²,
(42) —S(O)ₙR¹²,
(43) —SC(O)R¹²,
(44) —OC(O)OR¹²,
(45) —N(R¹²)OR¹²,
(46) —OC(═N(R¹²))R¹²,
(47) —N(R¹²)C(═NR¹²)R¹²,
(48) —C(O)OC(O)R¹²,
(49) ═N—N(R¹²)—C(O)C(O)N(R¹²)₂,
(50) ═NN(R¹²)C(S)N(R¹²)₂,
(51) ═C(R¹²)OR¹²,
(52) alkylidene,
(53) optionally substituted spiroalkyl,
(54) optionally substituted spiroheterocycle,
and
(55) ═N—N(R¹³)(R¹⁴)
wherein R¹² in (25)-(51) is independently selected from the group consisting of
(1) hydrogen,
(2) optionally substituted aryl,
(3) optionally substituted C₁-C₆ alkyl,
(4) optionally substituted C₃-C₆ alkenyl,
(5) optionally substituted aryl,
(6) optionally substituted arylalkyl,
and
(7) optionally substituted heterocycle,
and
wherein R¹³ and R¹⁴ in (55) together with the nitrogen atom to which they are attached form a heterocycle selected from the group consisting of pyrrolidinyl, piperidinyl, imidazolidinyl, pyrazolidinyl, piperazinyl, morpholinyl, and thiomorpholinyl, wherein the heterocycle defined by R¹³ and R¹⁴ together can be optionally substituted with optionally substituted alkyl.

2. A compound according to claim 1 wherein A¹ is methine.

3. A compound according to claim 1 wherein A² is —S—.

4. A compound according to claim 1 wherein R¹ is hydrogen.

5. A compound according to claim 1 wherein R¹ is fluoride.

6. A compound according to claim 1 wherein R¹⁵ is hydrogen.

7. A compound according to claim 1 wherein Z is 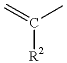
wherein R² is OR⁸ wherein R⁸ is methyl.

8. A compound according to claim 1 wherein Z is 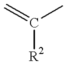
wherein R² is OR⁸ wherein R⁸ is difluoromethyl.

9. A compound according to claim 1 wherein R³ is cyclopropyl.

10. A compound according to claim 1 wherein R⁵ and R⁶ together are an optionally substituted carbocyclic ring.

11. A compound according to claim 1 wherein A¹ is methine, A² is —S—, R¹ is hydrogen, Z is 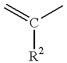
wherein R² is OR⁸ wherein R⁸ is methyl, R³ is cyclopropyl, R⁴ is OR¹¹ wherein R¹¹ is hydrogen, R¹⁵ is hydrogen, R⁵ and R⁶ together are an optionally substituted carbocyclic ring and the substituent is amino.

12. A compound according to claim 1 wherein R⁵ and R⁶ together are an optionally substituted heterocyclic ring.

13. A method for preparing a compound of formula (Ia), the method comprising
(a) reacting compounds of formula (Ia) 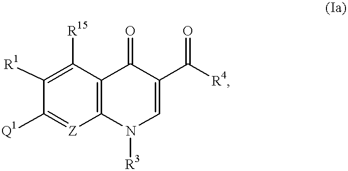
wherein R¹, R³, R⁴, R¹⁵, and Z are defined in claim 1, and Q¹ is a first covalent bond precursor, with compounds of formula (II) 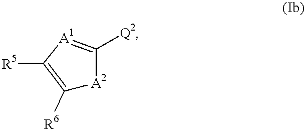
wherein A¹, A², and R⁵ and R⁶ are defined in claim 1, and Q² is a second covalent bond precursor, in the presence of a catalyst, to provide a first product;
and
(b) optionally hydrolyzing the first product.

14. The method according to claim 13, wherein Q¹ is selected from chloride, bromide, iodide, methanesulfonate, or trifluoromethanesulfonate.

15. The method according to claim 13, wherein Q² is a trialkylstannane, boronic acid, boronic ester, magnesium halide, zinc halide, or -silyl(alkyl)cyclobutane.

16. The method according to claim 13 wherein the catalyst is selected from tetrakis(triphenylphosphine)palladium(0), palladium(II) chloride(dibenzylidine acetone), or palladium(II) chloride bis(triphenylphosphine).

17. The method according to claim 14, wherein Q¹ is bromide.

18. The method according to claim 15, wherein Q² is a trialkylstannane.

19. A pharmaceutical composition comprising a pharmaceutically effective amount of a compound of claim 1 in combination with a pharmaceutically acceptable carrier.

20. A method of inhibiting the growth of bacteria which comprises contacting the bacteria with an pharmaceutically effective amount of a compound of claim 1.

21. A compound selected from the group consisting of
1-cyclopropyl-4-oxo-7-(4,5,6,7-tetrahydrothieno[3,2-c]pyridin-2-yl)-1,4-dihydro-3-quinolinecarboxylic acid,
1-cyclopropyl-8-methoxy-4-oxo-7-(4,5,6,7-tetrahydrothieno[3,2-c]pyridin-2-yl)-1,4-dihydro-3-quinolinecarboxylic acid,
1-cyclopropyl-4-oxo-7-(4,5,6,7-tetrahydrothieno[3,2-c]pyridin-2-yl)-1,4-dihydro[1,8]naphthyridine-3-carboxylic acid,
1-cyclopropyl-6-fluoro-4-oxo-7-(4,5,6,7-tetrahydrothieno[3,2-c]pyridin-2-yl)-1,4-dihydro[1,8]naphthyridine-3-carboxylic acid,
1-cyclopropyl-8-(difluoromethoxy)-4-oxo-7-(4,5,6,7-tetrahydrothieno[3,2-c]pyridin-2-yl)-1,4-dihydro-3-quinolinecarboxylic acid,
1-cyclopropyl-4-oxo-7-(4,5,6,7-tetrahydrothieno[2,3-c]pyridin-2-yl)-1,4-dihydro-3-quinolinecarboxylic acid,
1-cyclopropyl-8-methoxy-4-oxo-7-(4,5,6,7-tetrahydrothieno[2,3-c]pyridin-2-yl)-1,4-dihydro-3-quinolinecarboxylic acid,
1-cyclopropyl-4-oxo-7-(4,5,6,7-tetrahydrothieno[2,3-c]pyridin-2-yl)-1,4-dihydro[1,8]naphthyridine-3-carboxylic acid,
1-cyclopropyl-6-fluoro-4-oxo-7-(4,5,6,7-tetrahydrothieno[2,3-c]pyridin-2-yl)-1,4-dihydro[1,8]naphthyridine-3-carboxylic acid,
1-cyclopropyl-8-(difluoromethoxy)-4-oxo-7-(4,5,6,7-tetrahydrothieno[2,3-c]pyridin-2-yl)-1,4-dihydro-3-quinolinecarboxylic acid,
1-cyclopropyl-7-(6-methyl-4,5,6,7-tetrahydrothieno[3,2-c]pyridin-2-yl)-4-oxo-1,4-dihydro[1,8]naphthyridine-3-carboxylic acid,
1-cyclopropyl-6-fluoro-7-(6-methyl-4,5,6,7-tetrahydrothieno[3,2-c]pyridin-2-yl)-4-oxo-1,4-dihydro[1,8]naphthyridine-3-carboxylic acid,
1-cyclopropyl-7-(6-methyl-4,5,6,7-tetrahydrothieno[3,2-c]pyridin-2-yl)-4-oxo-1,4-dihydro-3-quinolinecarboxylic acid,
1-cyclopropyl-8-(difluoromethoxy)-7-(6-methyl-4,5,6,7-tetrahydrothieno[3,2-c]pyridin-2-yl)-4-oxo-1,4-dihydro-3-quinolinecarboxylic acid,
1-cyclopropyl-8-methoxy-7-(6-methyl-4,5,6,7-tetrahydrothieno[3,2-c]pyridin-2-yl)-4-oxo-1,4-dihydro-3-quinolinecarboxylic acid,
1-cyclopropyl-7-(4-methyl-4,5,6,7-tetrahydrothieno[3,2-c]pyridin-2-yl)-4-oxo-1,4-dihydro-3-quinolinecarboxylic acid,
1-cyclopropyl-7-(4-methyl-4,5,6,7-tetrahydrothieno[3,2-c]pyridin-2-yl)-4-oxo-1,4-dihydro[l,8]naphthyridine-3-carboxylic acid,
1-cyclopropyl-6-fluoro-7-(4-methyl-4,5,6,7-tetrahydrothieno[3,2-c]pyridin-2-yl)-4-oxo-1,4-dihydro[ 1,8]naphthyridine-3 -carboxylic acid,
1-cyclopropyl-8-methoxy-7-(4-methyl-4,5,6,7-tetrahydrothieno[3,2-c]pyridin-2-yl)-4-oxo-1,4-dihydro-3-quinolinecarboxylic acid,
1-cyclopropyl-8-(difluoromethoxy)-7-(4-methyl-4,5,6,7-tetrahydrothieno[3,2-c]pyridin-2-yl)-4-oxo-1,4-dihydro-3-quinolinecarboxylic acid,
1-cyclopropyl-7-(7-methyl-4,5,6,7-tetrahydrothieno[2,3-c]pyridin-2-yl)-4-oxo- 1,4-dihydro[1,8]naphthyridine-3-carboxylic acid,
1-cyclopropyl-6-fluoro-7-(7-methyl-4,5,6,7-tetrahydrothieno[2,3-c]pyridin-2-yl)-4-oxo-1,4-dihydro[1,8]naphthyridine-3-carboxylic acid,
1-cyclopropyl-8-methoxy-7-(7-methyl-4,5,6,7-tetrahydrotheino[2,3 -c]pyrindin-2-yl)-4-oxo-1,4-dihydro-3-quinolinecarboxylic acid,
1-cyclopropyl-7-(7,7-dimethyl-4,5,6,7-tetrahydrothieno[3 ,2-c]pyridin-2-yl)-4-oxo-1,4-dihydro[1,8]naphthyridine-3-carboxylic acid,
1-cyclopropyl-7-(7,7-dimethyl-4,5,6,7-tetrahydrothieno[3,2-c]pyridin-2-yl)-4-oxo-1,4-dihydro-3-quinolinecarboxylic acid,
1-cyclopropyl-7-(7,7-dimethyl-4,5,6,7-tetrahydrothieno [3,2-c]pyridin-2-yl)-8-methoxy-4-oxo-1,4-dihydro-3-quinolinecarboxylic acid,
1-cyclopropyl-7-(7,7-dimethyl-4,5,6,7-tetrahydrothieno[3,2-c]pyridin-2-yl)-6-fluoro-55 4-oxo-1,4-dihydro[1,8]naphthyridine-3-carboxylic acid,
1-cyclopropyl-8-(difluoromethoxy)-7-(7,7-dimethyl-4,5 ,6,7-tetrahydrothieno[3,2-c]pyridin-2-yl)-4-oxo-1,4-dihydro-3-quinolinecarboxylic acid,
1-cyclopropyl-7-(4-methyl-4,5,6,7-tetrahydrothieno[2,3 -c]pyridin-2-yl)-4-oxo- 1,4-dihydro-3-quinolinecarboxylic acid,
1-cyclopropyl-8-methoxy-7-(4-methyl-4,5,6,7-tetrahydrothieno[2,3-c]pyridin-2-yl)-4-oxo-1,4-dihydro-3-quinolinecarboxylic acid,
1-cyclopropyl-8-(difluoromethoxy)-7-(4-methyl-4,5,6,7-tetrahydrothieno[2,3-c]pyridin-2-yl)-4-oxo-1,4-dihydro-3-quinolinecarboxylic acid,
1-cyclopropyl-7-(4-methyl-4,5,6,7-tetrahydrothieno[2,3 -c]pyridin-2-yl)-4-oxo-1,4-dihydro[1,8]naphthyridine-3-carboxylic acid,
1-cyclopropyl-6-fluoro-7-(4-methyl-4,5,6,7-tetrahydrothieno[2,3-c]pyridin-2-yl)-4-oxo-1,4-dihydro[1,8]naphthyridine-3-carboxylic acid,
1-cyclopropyl-7-(4,4-dimethyl-4,5,6,7-tetrahydrothieno[2,3-c]pyridin-2-yl)-8-methoxy-4-oxo-1,4-dihydro-3-quinolinecarboxylic acid,
1-cyclopropyl-6-fluoro-7-(4-hydroxy-4,5,6,7-tetrahydro-1-benzothien-2-yl)-4-oxo-1,4-dihydro[1,8]naphthyridine-3-carboxylic acid,
1-cyclopropyl-7-(4-hydroxy-5,6-dihydro-4H-thieno[2,3-b]thiopyran-2-yl)-8-methoxy-4-oxo-1,4-dihydro-3-quinolinecarboxylic acid,
7-(4-amino-5,6-dihydro-4H-thieno[2,3 -b]thiopyran-2-yl)-1-cyclopropyl-8-methoxy-4-oxo-1,4-dihydro-3-quinolinecarboxylic acid,
1-cyclopropyl-7-(4-hydroxy-4,5,6,7-tetrahydro-1-benzothien-2-yl)-8-methoxy-4-oxo-1,4-dihydro-3-quinolinecarboxylic acid,
7-(4-azido-4,5,6,7-tetrahydro-1-benzothien-2-yl)-1-cyclopropyl-8-methoxy-4-oxo-1,4-dihydro-3-quinolinecarboxylic acid,
7-(4-amino-4,5,6,7-tetrahydro-1-benzothien-2-yl)- 1-cyclopropyl-8-methoxy-4-oxo-1,4-dihydro-3-quinolinecarboxylic acid,
1-cyclopropyl-7-(7-hydroxy-4,5,6,7-tetrahydro-1-benzothien-2-yl)-8-methoxy-4-oxo-1,4-dihydro-3-quinolinecarboxylic acid,
1-cyclopropyl-7-(5-hydroxy-4,5,6,7-tetrahydro-1-benzothien-2-yl)-8-methoxy-4-oxo-1,4-dihydro-3-quinolinecarboxylic acid,
1-cyclopropyl-7-(5-(hydroxymethyl)-4,5,6,7-tetrahydro-1-benzothien-2-yl)-8-methoxy-4-oxo-1,4-dihydro-3-quinolinecarboxylic acid,
1-cyclopropyl-7-(5-(hydroxymethyl)-6,7-dihydro-1-benzothien-2-yl)-8-methoxy-4-oxo-1,4-dihydro-3-quinolinecarboxylic acid,
1-cyclopropyl-7-(5-hydroxy-5,6-dihydro-4H-cyclopenta[b]thien-2-yl)-8-methoxy-4-oxo-1,4-dihydro-3-quinolinecarboxylic acid,
1-cyclopropyl-8-methoxy-7-(4-methoxy-5,6-dihydro-4H-cyclopenta[b]thien-2-yl)-4-oxo-1,4-dihydro-3-quinolinecarboxylic acid,
1-cyclopropyl-7-(6-hydroxy-4,5,6,7-tetrahydro-1-benzothien-2-yl)-8-methoxy-4-oxo-1,4-dihydro-3-quinolinecarboxylic acid,
1-cyclopropyl-6-fluoro-4-oxo-7-(4-oxo-4,5,6,7-tetrahydro-1-benzothien-2-yl)-1,4-dibydro[1,8]naphthyridine-3-carboxylic acid,
1-cyclopropyl-6-fluoro-7-((4E/Z)-4-(hydroxyimino)-4,5,6,7-tetrahydro-1-benzothien-2-yl)-4-oxo-1,4-dihydro(1,8)naphthyridine-3-carboxylic acid;
1-cyclopropyl-8-methoxy-4-oxo-7-(4-oxo-4,5,6,7-tetrahydro-1-benzothien-2-yl)-1,4-dihydro-3-quinolinecarboxylic acid;
1-cyclopropyl-7-((4E/Z)-4-(hydroxyimino)-4,5,6,7-tetrahydro-1-benzothien-2-yl)-8-methoxy-4-oxo-1,4-dihydro-3-quinolinecarboxylic acid;
1-cyclopropyl-8-diflouromethoxy-7-(7-methyl-4,5,6,7-tetrahydrotheino(2,3 -c)pyrindin-2-yl)-4-oxo-1,4-dihydro-3-quinolinecarboxylic acid;
1-cyclopropyl-8-methoxy-4-oxo-7-(5-oxo-4,5 ,6,7-tetrahydro-1-benzothien-2-yl)-1,4-dihydro-3-quinolinecarboxylic acid;
7-[5-(azidomethyl)-4,5,6,7-tetrahydro-1-benzothien-2-yl]-1-cyclopropyl-8-methoxy-4-oxo-1,4-dihydro-3-quinolinecarboxylic acid;
1-cyclopropyl-7-((5E/Z)-5-(hydroxyimino)-4,5,6,7-tetrahydro-1-benzothien-2-yl)-8-methoxy-4-oxo-1,4-dihydro-3-quinolinecarboxylic acid;
1-cyclopropyl-8-methoxy-7-((5E/Z)-5-(methoxyimino)-4,5 ,6,7-tetrahydro-1-benzothien-2-yl)-4-oxo-1,4-dihydro-3-quinolinecarboxylic acid;
1-cyclopropyl-7-(5-methyl-4,5,6,7-tetrahydrothieno[2,3-c]pyridin-2-yl)-4-oxo-1,4-dihydro[1,8]naphthyridine-3-carboxylic acid;
7-(5-bromo-4-oxo-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(6-oxo-4,5,6,7-tetrahydro-1-benzothien-2-yl)-1,4-dihydro-3-quinolinecarboxylic acid;
1-cyclopropyl-7-((6E/Z)-6-(hydroxyimino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(7-oxo-4,5 ,6,7-tetrahydro-1-benzothien-2-yl)- 1,4-dihydro-3-quinolinecarboxylic acid;
7-(5-azido-4-oxo-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((7E/Z)-7-(methoxyimino)-4,5,6,7-tetrahydro-1-benzothien-2-yl)-4-oxo-1,4-dihydro-3-quinolinecarboxylic acid;
1-cyclopropyl-8-methoxy-7-((4E/Z)-4-(methoxyimino)-4,5 ,6,7-tetrahydro-1-benzothien-2-yl)-4-oxo-1,4-dihydro-3-quinolinecarboxylic acid;
1-cyclopropyl-7-((4E/Z)-4-(ethoxyimino)-4,5,6,7-tetrahydro-1-benzothien-2-yl)-8-methoxy-4-oxo-1,4-dihydro-3-quinolinecarboxylic acid;
1-cyclopropyl-8-methoxy-7-((6E/Z)-6-(methoxyimino)-4,5,6,7-tetrahydro-1-benzothien-2-yl)-4-oxo-1,4-dihydro-3-quinolinecarboxylic acid;
1-cyclopropyl-7-(6,7-dihydro-1-befizothien-2-yl)-8-methoxy-4-oxo-1,4-dihydro-3-quinolinecarboxylic acid;
1-cyclopropyl-8-methoxy-7-((4E/Z)-4-(4-morpholinylimino)-4,5,6,7-tetrahydro-1-benzothien-2-yl)-4-oxo-1,4-dihydro-3-quinolinecarboxylic acid;
1-cyclopropyl-7-(4,5-dihydroxy-4,5,6,7-tetrahydro-1-benzothien-2-yl)-8-methoxy-4-oxo-1,4-dihydro-3-quinolinecarboxylic acid;
7-(5-(aminomethyl)-4,5,6,7-tetrahydro-1-benzothien-2-yl)-1-cyclopropyl-8-methoxy-4-oxo-1,4-dihydro-3-quinolinecarboxylic acid;
7-(6-amino-4,5,6,7-tetrahydro-1-benzothien-2-yl)-1-cyclopropyl-8-methoxy-4-oxo-1,4-dihydro-3-quinolinecarboxylic acid;
7-((4E/Z)-4-(tert-butoxyimino)-4,5,6,7-tetrahydro-1-benzothien-2-yl)-1-cyclopropyl-8-methoxy-4-oxo-1,4-dihydro-3-quinolinecarboxylic acid;
7-((4E/Z)-4-((benzyloxy)imino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-((4E/Z)-4-(1-pyrrolidinylimino)-4,5,6,7-tetrahydro-1-benzothien-2-yl)-1,4-dihydro-3-quinolinecarboxylic acid;
7-(4-amino-4,5,6,7-tetrahydro-1-benzothien-3-yl)-1-cyclopropyl-8-methoxy-4-oxo-1,4-dihydro-3-quinolinecarboxylic acid;
7-(5-amino-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-[4-(methylamino)-4,5,6,7-tetrahydro-1-benzothien-2-yl]-4-oxo-1,4-dihydro-3-quinolinecarboxylic acid;
1-cyclopropyl-7-((5E/Z)-5-(ethoxyimino)-4,5,6,7-tetrahydro-1-benzothien-2-yl)-8-methoxy-4-oxo-1,4-dihydro-3-quinolinecarboxylic acid;
7-((5E/Z)-5-((benzyloxy)imino)-4,5,6,7-tetrahydro-1-benzothien-2-yl)-1-cyclopropyl-8-methoxy-4-oxo-1,4-dihydro-3-quinolinecarboxylic acid;
7-((4E/Z)-4-((aminocarbonyl)hydrazono)-4,5,6,7-tetrahydro-1-benzothien-2-yl)1-cyclopropyl-8-methoxy-4-oxo-1,4-dihydro-3-quinolinecarboxylic acid;
ethyl 1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3 -quinolinecarboxylate;
ethyl 1-cyclopropyl-8-methoxy-7-((4E/Z)-4-(((2R)-2-(methoxymethyl)pyrrolidinyl)imino)-4,5,6,7-tetrahydro-1-benzothien-2-yl)-4-oxo-1,4-dihydro-3-quinolinecarboxylate;
1-cyclopropyl-7-(4-(dimethylamino)-4,5,6,7-tetrahydro-1-benzothien-2-yl)-8-methoxy-4-oxo-1,4-dihydro-3-quinolinecarboxylic acid;
7-((4E/Z)-4-[(aminocarbothioyl)hydrazono)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((4E/Z)-4-(((methylamino)carbothioyl)hydrazono)-4,5,6,7-tetrahydro-1-benzothien-2-yl)-4-oxo-1,4-dihydro-3-quinolinecarboxylic acid;
1-cyclopropyl-8-methoxy-7-(5 -methylene-4-oxo-4,5,6,7-tetrahydro-1-benzothien-2-yl)-4-oxo-1,4-dihydro-3-quinolinecarboxylic acid;
ethyl 1-cyclopropyl-8-methoxy-7-(5-methylene-4-oxo-4,5,6,7-tetrahydro-1-benzothien-2-yl)-4-oxo-1,4-dihydro-3-quinolinecarboxylate;
1-cyclopropyl-8-methoxy-7-(4-((methylsulfonyl)amino)-4,5 ,6,7-tetrahydro-1-benzothien-2-yl)-4-oxo-1,4-dihydro-3-quinolinecarboxylic acid;
1-cyclopropyl-8-methoxy-4-oxo-7-(4-(1H-pyrrol-1-yl)-4,5,6,7-tetrahydro-1-benzothien-2-yl)-1,4-dihydro-3-quinolinecarboxylic acid;
1-cyclopropyl-7-((4E/Z)-4-(((ethylamino)carbothioyl)hydrazono)-4,5,6,7-tetrahydro-1-benzothien-2-yl)-8-methoxy-4-oxo-1,4-dihydro-3-quinolinecarboxylic acid;
7-((4E/Z)-4-((amino(oxo)acetyl)hydrazono)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylic acid;
7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((3-pyridinylmethyl)amino)-4,5,6,7-tetrahydro-1-benzothien-2-yl)-1,4-dihydro-3-quinolinecarboxylic acid;
ethyl 7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylate;
7-((4E/Z)-4-(acetylhydrazono)-4,5,6,7-tetrahydro-1-benzothien-2-yl)-1-cyclopropyl-8-methoxy-4-oxo-1,4-dihydro-3-quinolinecarboxylic acid;
7-(4-(benzylamino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-(ethylamino)-4,5,6,7-tetrahydro-1-benzothien-2-yl)-8-methoxy-4-oxo-1,4-dihydro-3-quinolinecarboxylic acid;
1-cyclopropyl-8-(difluoromethoxy)-7-(4-hydroxy-4,5,6,7-tetrahydro-1-benzothien-2-yl)-4-oxo-1,4-dihydro-3-quinolinecarboxylic acid;
1-cyclopropyl-7-((4E/Z)-4-(4,5-dihydro-1H-imidazol-2-ylhydrazono)-4,5,6,7-tetrahydro- 1 -benzothien-2-yl)-8-methoxy-4-oxo- 1,4-dihydro-3-quinolinecarboxylic acid;
7-(4-amino-4,5,6,7-tetrahydro-1-benzothien-2-yl)-1-cyclopropyl-8-(difluoromethoxy)-4-oxo-1,4-dihydro-3-quinolinecarboxylic acid;
1-cyclopropyl-7-(4-hydroxy-2-methyl-1,1-dioxido-3,4-dihydro-2H-thieno[3,2-e][1 ,2]thiazin-6-yl)-8-methoxy-4-oxo-1,4-dihydro-3-quinolinecarboxylic acid;
ethyl 1-cyclopropyl-8-methoxy-7-(5-methyl-4-oxo-4,5,6,7-tetrahydro-1-benzothien-2-yl)-4-oxo- 1 ,4-dihydro-3 -quinolinecarboxylate;
1-cyclopropyl-7-(4-((3-fluorobenzyl)amino)-4,5,6,7-tetrahydro-1-benzothien-2-yl)-8-methoxy-4-oxo-1,4-dihydro-3-quinolinecarboxylic acid;
7-((4E/Z)-4-[(aminocarbothioyl)(methyl)hydrazono)-4,5,6,7-tetrahydro-1-benzothien-2-yl)- 1 -cyclopropyl-8-methoxy-4-oxo-1,4-dihydro-3-quinolinecarboxylic acid;
1-cyclopropyl-8-methoxy-7-(5-methyl-4-oxo-4,5,6,7-tetrahydro-1-benzothien-2-yl)-4-oxo-1,4-dihydro-3-quinolinecarboxylic acid;
1-cyclopropyl-8-(difluoromethoxy)-7-(4-(methylamino)-4,5,6,7-tetrahydro-1-benzothien-2-yl)-4-oxo-1,4-dihydro-3-quinolinecarboxylic acid;
1-cyclopropyl-6-fluoro-7-(4-(methylamino)-4,5,6,7-tetrahydro- 1 -benzothien-2-yl)-4-oxo-1,4-dihydro[1,8]naphthyridine-3-carboxylic acid;
7-(4-amino-2-methyl-1,1-dioxido-3,4-dihydro-2H-thieno[3,2-e][1,2]thiazin-6-yl)-1-cyclopropyl-8-methoxy-4-oxo-1,4-dihydro-3-quinolinecarboxylic acid;
1-cyclopropyl-7-(4-(hydroxymethyl)-4,5,6,7-tetrahydro- 1 -benzothien-2-yl)-8-methoxy-4-oxo-1,4-dihydro-3-quinolinecarboxylic acid;
1-cyclopropyl-8-methoxy-4-oxo-7-(4-(1-pyrrolidinylmethyl)-4,5,6,7-tetrahydro-1-benzothien-2-yl)-1,4-dihydro-3-quinolinecarboxylic acid;
1-cyclopropyl-8-methoxy-4-oxo-7-(4-((2-pyrrolidinylmethyl)amino)-4,5,6,7-tetrahydro-1-benzothien-2-yl)- 1,4-dihydro-3-quinolinecarboxylic acid;
7-(4-(acetylamino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-(propionylamino)-4,5,6,7-tetrahydro-1-benzothien-2-yl)-1,4-dihydro-3-quinolinecarboxylic acid;
1-cyclopropyl-8-methoxy-7-(4-((methoxyacetyl)amino)-4,5,6,7-tetrahydro-1-benzothien-2-yl)-4-oxo-1,4-dihydro-3-quinolinecarboxylic acid;
1-cyclopropyl-8-methoxy-4-oxo-7-(4-((tetrahydrofuranyl-2-carbonyl)amino)-4,5,6,7-tetrahydro- 1 -benzothien-2-yl)-1,4-dihydro-3-quinolinecarboxylic acid;
1-cyclopropyl-8-methoxy-4-oxo-7-(4-((tetrahydrofuranyl-3-carbonyl)amino)-4,5,6,7-tetrahydro- 1 -benzothien-2-yl)-1,4-dihydro-3-quinolinecarboxylic acid;
1-cyclopropyl-8-methoxy-7-(4-((4-morpholinylacetyl)amino)-4,5,6,7-tetrahydro-1-benzothien-2-yl)-4-oxo-1,4-dihydro-3-quinolinecarboxylic acid;
1-cyclopropyl-8-methoxy-7-(4-((3-(4-morpholinyl)propanoyl)amino)-4,5,6,7-tetrahydro- 1 -benzothien-2-yl)-4-oxo-1,4-dihydro-3-quinolinecarboxylic acid;
1-cyclopropyl-8-methoxy-4-oxo-7-(4-((1H-pyrrol-2-ylcarbonyl)amino)-4,5,6,7-tetrahydro- 1 -benzothien-2-yl)-1,4-dihydro-3-quinolinecarboxylic acid;
1-cyclopropyl-8-methoxy-4-oxo-7-(4-((3-pyridinylacetyl)amino)-4,5,6,7-tetrahydro-1-benzothien-2-yl)-1,4-dihydro-3-quinolinecarboxylic acid;
1-cyclopropyl-8-methoxy-4-oxo-7-(4-((3-pyridazinylcarbonyl)amino)-4,5,6,7-tetrahydro-1-benzothien-2-yl)- 1,4-dihydro-3 -quinolinecarboxylic acid;
1-cyclopropyl-7-(4-((1H-imidazol-2-ylcarbonyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((1,3-thiazol-2-ylcarbonyl)amino)-4,5,6,7-tetrahydro-1-benzothien-2-yl)-1,4-dihydro-3-quinolinecarboxylic acid;
1-cyclopropyl-8-methoxy-7-(5-(methylamino)-4,5,6,7-tetrahydro-1-benzothien-2-yl)-4-oxo-1,4-dihydro-3-quinolinecarboxylic acid;
1-cyclopropyl-8-methoxy-7-(4-(4-morpholinylmethyl)-4,5,6,7-tetrahydro-1-benzothien-2-yl)-4-oxo-1,4-dihydro-3-quinolinecarboxylic acid;
1-cyclopropyl-7-(4-((dimethylamino)methyl)-4,5,6,7-tetrahydro-1-benzothien-2-yl)-8-methoxy-4-oxo-1,4-dihydro-3-quinolinecarboxylic acid;
1-cyclopropyl-7-(4-(((dimethylamino)acetyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((2-pyridinylacetyl)amino)-4,5 ,6,7-tetrahydro-1-benzothien-2-yl)-1,4-dihydro-3-quinolinecarboxylic acid;
7-(4-(aminomethyl)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-((4E/Z)-4-((4-pyridinylmethoxy)imino)-4,5,6,7-tetrahydro-1-benzothien-2-yl)-1,4-dihydro-3-quinolinecarboxylic acid;
7-(4-((2-aminoethyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-(4-((1 -methyl-4-piperidinyl)amino)-4,5,6,7-tetrahydro-1-benzothien-2-yl)-4-oxo-1,4-dihydro-3-quinolinecarboxylic acid;
1-cyclopropyl-7-((4E/Z)-4-(hydroxyimino)-5-methyl-4,5,6,7-tetrahydro-1-benzothien-2-yl)-8-methoxy-4-oxo-1,4-dihydro-3-quinolinecarboxylic acid;
1-cyclopropyl-7-(4-hydroxy-7,7-dioxido-5,6-dihydro-4H-thieno[2,3-b]thiopyran-2-yl)-8-methoxy-4-oxo-1,4-dihydro-3-quinolinecarboxylic acid;
7-(4-(((5-chloro- 1,3-dimethyl-1H-pyrazol-4-yl)sulfonyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((4-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((phenylsulfonyl)amino)-4,5,6,7-tetrahydro-1-benzothien-2-yl)-1,4-dihydro-3-quinolinecarboxylic acid;
7-(4-(((2-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-(4-(((4-methoxyphenyl)sulfonyl)amino)-4,5,6,7-tetrahydro-1-benzothien-2-yl)-4-oxo-1,4-dihydro-3-quinolinecarboxylic acid;
1-cyclopropyl-8-methoxy-7-(4-(((3-nitrophenyl)sulfonyl)amino)-4,5,6,7-tetrahydro- 1-benzothien-2-yl)-4-oxo-1,4-dihydro-3-quinolinecarboxylic acid;
1-cyclopropyl-8-methoxy-4-oxo-7-(5-((2-pyrrolidinylmethyl)amino)-4,5,6,7-tetrahydro-1-benzothien-2-yl)-1,4-dihydro-3-quinolinecarboxylic acid;
7-(4-amino-7,7-dioxido-5,6-dihydro-4H-thieno[2,3-b]thiopyran-2-yl)-1-cyclopropyl-8-methoxy-4-oxo-1,4-dihydro-3-quinolinecarboxylic acid;
1-cyclopropyl-7-(4-(((3,5-dimethyl-4-isoxazolyl)sulfonyl)amino)-4,5,6,7-tetrahydro-1-benzothien-2-yl)-8-methoxy-4-oxo-1,4-dihydro-3-quinolinecarboxylic acid;
7-(4-((2,1,3-benzoxadiazol-4-ylsulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-(((dimethylamino)sulfonyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((2-thienylsulfonyl)amino)-4,5,6,7-tetrahydro-1-benzothien-2-yl)-1,4-dihydro-3-quinolinecarboxylic acid;
7-(4-(((3-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((4-(acetylamino)phenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
7-(4-((2,1,3-benzothiadiazol-4-ylsulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-(((5-(3-isoxazolyl)-2-thienyl)sulfonyl)amino)-4,5,6,7-tetrahydro-1-benzothien-2-yl)-8-methoxy-4-oxo-1,4-dihydro-3-quinolinecarboxylic acid;
1-cyclopropyl-7-(4-(((4-fluorophenyl)sulfonyl)amino)-4,5,6,7-tetrahydro-1-benzothien-2-yl)-8-methoxy-4-oxo-1,4-dihydro-3-quinolinecarboxylic acid;
7-(4-(((6-chloroimidazo[2,1-b][1,3]thiazol-5-yl)sulfonyl)amino)-4,5,6,7-tetrahydro-1-benzothien-2-yl) 1 -cyclopropyl-8-methoxy-4-oxo-1,4-dihydro-3 -quinolinecarboxylic acid;
1-cyclopropyl-8-methoxy-4-oxo-7-(4-((4-pyridinylacetyl)amino)-4,5,6,7-tetrahydro-1-benzothien-2-yl)-1,4-dihydro-3-quinolinecarboxylic acid;
1-cyclopropyl-7-(4-((2-hydroxyethyl)amino)-4,5,6,7-tetrahydro-1-benzothien-2-yl)-8-methoxy-4-oxo-1,4-dihydro-3-quinolinecarboxylic acid;
7-(4-amino-4,5,6,7-tetrahydro-1-benzothien-2-yl)-1-cyclopropyl-6-fluoro-4-oxo-1,4-dihydro[1,8]naphthyridine-3-carboxylic acid;
7-(4-((glycyl)amino)-4,5,6,7-tetrahydro-1-benzothien-2-yl)-1-cyclopropyl-8-methoxy-4-oxo-1,4-dihydro-3-quinolinecarboxylic acid;
7-(4-((D-alanyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((D-prolyl)amino)-4,5,6,7-tetrahydro-1-benzothien-2-yl)-1,4-dihydro-3-quinolinecarboxylic acid;
7-(4-(((2R)-2-amino-3-(1H-imidazol-5-yl)propanoyl)amino)-4,5,6,7-tetrahydro-1-benzothien-2-yl)-1-cyclopropyl-8-methoxy-4-oxo-1,4-dihydro-3-quinolinecarboxylic acid;
7-(4-((leucyl)amino)-4,5,6,7-tetrahydro-1-benzothien-2-yl)-1-cyclopropyl-8-methoxy-4-oxo-1,4-dihydro-3-quinolinecarboxylic acid;
7-(4-((D-tyrosyl)amino)-4,5,6,7-tetrahydro-1-benzothien-2-yl)-1-cyclopropyl-8-methoxy-4-oxo-1,4-dihydro-3-quinolinecarboxylic acid;
7-(4-((O-methyl-D-tyrosyl)amino)-4,5,6,7-tetrahydro-1-benzothien-2-yl)-1-cyclopropyl-8-methoxy-4-oxo-1,4-dihydro-3-quinolinecarboxylic acid;
7-(4-((D-methionyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((2R)-2-amino-3-(3-pyridinyl)propanoyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-(((2R)-piperidinylcarbonyl)amino)-4,5,6,7-tetrahydro-1-benzothien-2-yl)-1,4-dihydro-3-quinolinecarboxylic acid;
1-cyclopropyl-8-methoxy-4-oxo-7-(4-((4-pyrimidinylcarbonyl)amino)-4,5,6,7-tetrahydro-1-benzothien-2-yl)-1,4-dihydro-3-quinolinecarboxylic acid;
1-cyclopropyl-8-methoxy-4-oxo-7-(4-(( 1,3-thiazol-2-ylcarbonyl)amino)-4,5,6,7-tetrahydro-1-benzothien-2-yl)-1,4-dihydro-3-quinolinecarboxylic acid;
1-cyclopropyl-8-methoxy-4-oxo-7-(4-((phenylacetyl)amino)-4,5,6,7-tetrahydro-1-benzothien-2-yl)-1,4-dihydro-3-quinolinecarboxylic acid;
1-cyclopropyl-7-(4-(3-furoylamino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((2-pyridinylcarbonyl)amino)-4,5,6,7-tetrahydro-1-benzothien-2-yl)-1,4-dihydro-3-quinolinecarboxylic acid;
1-cyclopropyl-8-methoxy-4-oxo-7-(4-((1H-pyrazol-4-ylcarbonyl)amino)-4,5,6,7-tetrahydro-1-benzothien-2-yl)-1,4-dihydro-3-quinolinecarboxylic acid;
7-(4-((D-aspartyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((4R)-4-((N-methyl-D-leucyl)amino)-4,5,6,7-tetrahydro-1-benzothien-2-yl)-4-oxo-1,4-dihydro-3-quinolinecarboxylic acid;
7-((4R)-4-((D-norleucyl)amino)-4,5,6,7-tetrahydro-1-benzothien-2-yl)-1-cyclopropyl-8-methoxy-4-oxo-1,4-dihydro-3-quinolinecarboxylic acid;
methyl 1-cyclopropyl-8-methoxy-4-oxo-7-(4-((4-pyrimidinylcarbonyl)amino)-4,5,6,7-tetrahydro-1-benzothien-2-yl)-1,4-dihydro-3-quinolinecarboxylate;
methyl 1-cyclopropyl-8-methoxy-4-oxo-7-(4-((1,3-thiazol-2-ylcarbonyl)amino)-4,5,6,7-tetrahydro-1-benzothien-2-yl)-1,4-dihydro-3-quinolinecarboxylate;
methyl 1-cyclopropyl-8-methoxy-4-oxo-7-(4-((2-pyridinylcarbonyl)amino)-4,5,6,7-tetrahydro-1-benzothien-2-yl)-1,4-dihydro-3-quinolinecarboxylate;
7-(4-((β-O-methyl-D-aspartyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-((4E)-4-((3-pyridinylmethoxy)imino)-4,5,6,7-tetrahydro- 1 -benzothien-2-yl)-1,4-dihydro-3 -quinolinecarboxylic acid;
1-cyclopropyl-8-methoxy-4-oxo-7-((4E)-4-((2-pyridinylmethoxy)imino)-4,5,6,7-tetrahydro- 1 -benzothien-2-yl)-1,4-dihydro-3 -quinolinecarboxylic acid;
ethyl 7-(4-((tert-butyl(dimethyl)silyl)oxy)-5-methyl-4,5,6,7-tetrahydro-1-benzothien-2-yl)- 1 -cyclopropyl-8-methoxy-4-oxo-1,4-dihydro-3-quinolinecarboxylate;
1-cyclopropyl-7-(4-hydroxy-5-methyl-4,5,6,7-tetrahydro-1-benzothien-2-yl)-8-methoxy-4-oxo-1,4-dihydro-3-quinolinecarboxylic acid;
1-cyclopropyl-8-methoxy-7-(4-(((methylanilino)carbonyl)amino)-4,5,6,7-tetrahydro-1-benzothien-2-yl)-4-oxo-1,4-dihydro-3-quinolinecarboxylic acid;
1-cyclopropyl-7-(4-(((diethylamino)carbonyl)amino)-4,5,6,7-tetrahydro-1-benzothien-2-yl)-8-methoxy-4-oxo-1,4-dihydro-3-quinolinecarboxylic acid;
1-cyclopropyl-7-(4-(((diisopropylamino)carbonyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-7-(4-((4-morpholinylcarbonyl)amino)-4,5,6,7-tetrahydro-1-benzothien-2-yl)-4-oxo-1,4-dihydro-3-quinolinecarboxylic acid;
1-cyclopropyl-8-methoxy-7-(4-((methoxycarbonyl)amino)-4,5,6,7-tetrahydro-1-benzothien-2-yl)-4-oxo-1,4-dihydro-3-quinolinecarboxylic acid;
7-(4-(((benzyloxy)carbonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-((isobutoxycarbonyl)amino)-4,5,6,7-tetrahydro-1-benzothien-2-yl)-8-methoxy-4-oxo-1,4-dihydro-3-quinolinecarboxylic acid;
1-cyclopropyl-7-(4-((ethoxycarbonyl)amino)-4,5,6,7-tetrahydro- 1 -benzothien-2-yl)-8-methoxy-4-oxo-1,4-dihydro-3-quinolinecarboxylic acid;
7-(4-((butoxycarbonyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((4-chlorobutoxy)carbonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-((5E/Z)-5-(hydroxymethylene)-4-oxo-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-((4E/Z)-4-((3 -pyridinylmethoxy)imino)-4,5,6,7-tetrahydro-1-benzothien-2-yl)-1,4-dihydro-3-quinolinecarboxylic acid;
1-cyclopropyl-7-(4-(3-hydroxy- 1 -azetidinyl)-4,5,6,7-tetrahydro-1-benzothien-2-yl)-8-methoxy-4-oxo-1,4-dihydro-3-quinolinecarboxylic acid;
7-(4-amino-5 -methyl-4,5,6,7-tetrahydro- 1 -benzothien-2-yl)-1-cyclopropyl-8-methoxy-4-oxo-1,4-dihydro-3-quinolinecarboxylic acid;
7-(4-amino-4,5,6,7-tetrahydro-1-benzothien-2-yl)-1-cyclopropyl-4-oxo-1,4-dihydro[1,8]naphthyridine-3-carboxylic acid;
7-(7-amino-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(7-hydroxy-4,5,6,7-tetrahydrothieno[3 ,2-c]pyridin-2-yl)-8-methoxy-4-oxo-1,4-dihydro-3-quinolinecarboxylic acid;
1-cyclopropyl-8-methoxy-4-oxo-7-(7-oxo-4,5,6,7-tetrahydrothieno[3,2-c]pyridin-2-yl)-1,4-dihydro-3-quinolinecarboxylic acid;
1-cyclopropyl-7-(5,5-difluoro-4-hydroxy-4,5,6,7-tetrahydro-1-benzothien-2-yl)-8-methoxy-4-oxo-1,4-dihydro-3-quinolinecarboxylic acid;
ethyl 7-(4-azido-5-methyl-4,5,6,7-tetrahydro-1-benzothien-2-yl)-1-cyclopropyl-8-methoxy-4-oxo-1,4-dihydro-3-quinolinecarboxylate;
7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(5-(hydroxymethyl)-5-methyl-4-oxo-4,5,6,7-tetrahydro-1-benzothien-2-yl)-8-methoxy-4-oxo-1,4-dihydro-3-quinolinecarboxylic acid;
7-(7-amino-4,5,6,7-tetrahydrothieno[3,2-c]pyridin-2-yl)-1-cyclopropyl-8-methoxy-4-oxo-1,4-dihydro-3-quinolinecarboxylic acid;
1-cyclopropyl-7-(4-hydroxy-4,5,6,7-tetrahydrothieno[2,3-c]pyridin-2-yl)-8-methoxy-4-oxo-1,4-dihydro-3-quinolinecarboxylic acid;
1-cyclopropyl-6-fluoro-8-methoxy-7-(4-(methylamino)-4,5,6,7-tetrahydro-1-benzothien-2-yl)-4-oxo-1,4-dihydro-3-quinolinecarboxylic acid hydrochloride;
1-cyclopropyl-8-methoxy-4-oxo-7-(4-oxo-4,5 ,6,7-tetrahydrothieno [2,3-c]pyridin-2-yl)-1,4-dihydro-3-quinolinecarboxylic acid;
7-[4-(3-amino-1-azetidinyl)-4,5,6,7-tetrahydro- 1 -benzothien-2-yl]-1-cyclopropyl-8-methoxy-4-oxo- 1 ,4-dihydro-3-quinolinecarboxylic acid;
1-cyclopropyl-7-(4-hydroxy-5,5-dimethyl-4,5,6,7-tetrahydro-1-benzothien-2-yl)-8-methoxy-4-oxo-1,4-dihydro-3-quinolinecarboxylic acid;
7-(4-((tert-butoxycarbonyl)amino)-5-methyl-4,5,6,7-tetrahydro-1-benzothien-2-yl)-1-cyclopropyl-8-methoxy-4-oxo-1,4-dihydro-3-quinolinecarboxylic acid;
methyl 7-[4-(acetyloxy)-4,5,6,7-tetrahydro-1-benzothien-2-yl]-1-cyclopropyl-8-methoxy-4-oxo-1,4-dihydro-3-quinolinecarboxylate;
1-cyclopropyl-8-methoxy-7-(7-(methylamino)-4,5,6,7-tetrahydro- 1-benzothien-2-yl)-4-oxo-1,4-dihydro-3-quinolinecarboxylic acid;
methyl 1-cyclopropyl-6-fluoro-7-(4-hydroxy-4,5,6,7-tetrahydro-1-benzothien-2-yl)-8-methoxy-4-oxo-1 ,4-dihydro-3-quinolinecarboxylate;
1-cyclopropyl-7-(7-hydroxy-4,4-dimethyl-4,5,6,7-tetrahydro-1-benzothien-2-yl)-8-methoxy-4-oxo-1,4-dihydro-3-quinolinecarboxylic acid;
methyl 7-(4-azido-4,5,6,7-tetrahydro-1-benzothien-2-yl)-1-cyclopropyl-6-fluoro-8-methoxy-4-oxo-1,4-dihydro-3-quinolinecarboxylate;
methyl 7-(4-((tert-butoxycarbonyl)amino)-4,5,6,7-tetrahydro-1-benzothien-2-yl)-1-cyclopropyl-6-fluoro-8-methoxy-4-oxo-1,4-dihydro-3-quinolinecarboxylate;
7-(4-((tert-butoxycarbonyl)amino)-4,5,6,7-tetrahydro-1-benzothien-2-yl)-1-cyclopropyl-6-fluoro-8-methoxy-4-oxo-1,4-dihydro-3-quinolinecarboxylic acid;
7-(7-amino-4,4-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-oxo-5-spiro-3′-(N-benzylpyrrolidine)-4,5,6,7-tetrahydro-1-benzothien-2-yl)-1,4-dihydro-3-quinolinecarboxylic acid;
7-(4-amino-4,5,6,7-tetrahydro-1-benzothien-2-yl)-1-cyclopropyl-6-fluoro-8-methoxy-4-oxo-1,4-dihydro-3-quinolinecarboxylic acid;
7-(4-amino-4,5,6,7-tetrahydrothieno[2,3-c]pyridin-2-yl)-1-cyclopropyl-8-methoxy-4-oxo-1,4-dihydro-3-quinolinecarboxylic acid;
7-(7-azido-4,5,6,7-tetrahydrothieno[3,2-c]pyridin-2-yl)-1-cyclopropyl-8-methoxy-4-oxo-1,4-dihydro-3-quinolinecarboxylic acid;
1-cyclopropyl-7-(4,4-dimethyl-4,5,6,7-tetrahydro-1-benzothien-2-yl)-8-methoxy-4-oxo-1,4-dihydro-3 -quinolinecarboxylic acid;
7-(7-amino-4,5,6,7-tetrahydro-1-benzothien-2-yl)- 1 -cyclopropyl-6-fluoro-8-methoxy-4-oxo-1,4-dihydro-3-quinolinecarboxylic acid;
7-(5-acetyl-7-amino-4,5,6,7-tetrahydrothieno[3,2-c]pyridin-2-yl)-1-cyclopropyl-8-methoxy-4-oxo-1,4-dihydro-3-quinolinecarboxylic acid;
7-(7-amino-5-(methylsulfonyl)-4,5,6,7-tetrahydrothieno[3,2-c]pyridin-2-yl)-1-cyclopropyl-8-methoxy-4-oxo-1,4-dihydro-3-quinolinecarboxylic acid;
7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid;
7-(4-amino-3-methyl-4,5,6,7-tetrahydro-1-benzothien-2-yl)-1-cyclopropyl-8-methoxy-4-oxo-1,4-dihydro-3-quinolinecarboxylic acid;
1-cyclopropyl-7-((4E/Z)-4-(hydroxyimino)-5,5-dimethyl-4,5,6,7-tetrahydro-1-benzothien-2-yl)-8-methoxy-4-oxo-1,4-dihydro-3-quinolinecarboxylic acid;
1-cyclopropyl-7-(4-hydroxy-6,6-dimethyl-4,5,6,7-tetrahydro-1-benzothien-2-yl)-8-methoxy-4-oxo-1,4-dihydro-3-quinolinecarboxylic acid;
7-(7-amino-6-methyl-4,5,6,7-tetrahydro-1-benzothien-2-yl)-1-cyclopropyl-8-methoxy-4-oxo- 1,4-dihydro-3-quinolinecarboxylic acid hydrochloride;
1-cyclopropyl-7-(6,6-dimethyl-4,5,6,7-tetrahydro-1-benzothien-2-yl)-8-methoxy-4-oxo-1,4-dihydro-3-quinolinecarboxylic acid;
7-(7-amino-6-fluoro-4,5,6,7-tetrahydro-1-benzothien-2-yl)-1-cyclopropyl-8-methoxy-4-oxo-1,4-dihydro-3-quinolinecarboxylic acid;
7-(4-amino-6,6-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(7-oxo-4,5 ,6,7-tetrahydro- 1,3-benzothiazol-2-yl)-1,4-dihydro-3-quinolinecarboxylic acid;
1-cyclopropyl-7-(6-fluoro-7-hydroxy-4,5,6,7-tetrahydro-1-benzothien-2-yl)-8-methoxy-4-oxo-1,4-dihydro-3-quinolinecarboxylic acid;
1-cyclopropyl-7-(7-hydroxy-6,6-dimethyl-4,5,6,7-tetrahydro-1-benzothien-2-yl)-8-methoxy-4-oxo-1,4-dihydro-3-quinolinecarboxylic acid;
1-cyclopropyl-7-((6S,7S)-7-hydroxy-6-methyl-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4,5,6,7-tetrahydro-1-benzothien-2-yl)-1,4-dihydro-3-quinolinecarboxylic acid;
7-(7-amino-6,6-difluoro-4,5,6,7-tetrahydro-1-benzothien-2-yl)-1-cyclopropyl-8-methoxy-4-oxo-1,4-dihydro-3-quinolinecarboxylic acid;
7-(7-amino-6,6-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-6-fluoro-8-methoxy-7-(7-(methylamino)-4,5,6,7-tetrahydro-1-benzothien-2-yl)-4-oxo-1,4-dihydro-3-quinolinecarboxylic acid;
1-cyclopropyl-8-methoxy-4-oxo-7-(-oxo-5,6-dihydro-4H-cyclopenta[b]thien-2-yl)-1,4-dihydro-3-quinolinecarboxylic acid;
1-cyclopropyl-8-methoxy-7-((5E/Z)-5-(methoxyimino)-5,6-dihydro-4H-cyclopenta[b]thien-2-yl)-4-oxo-1,4-dihydro-3-quinolinecarboxylic acid;
7-(5-amino-5,6-dihydro-4H-cyclopenta[b]thien-2-yl)-1-cyclopropyl-8-methoxy-4-oxo-1,4-dihydro-3-quinolinecarboxylic acid;
1-cyclopropyl-7-(5-((ethoxycarbonyl)amino)-S5,6-dihydro-4H-cyclopenta[b]thien-2-yl)-8-methoxy-4-oxo-1,4-dihydro-3-quinolinecarboxylic acid;
1-cyclopropyl-8-methoxy-7-(5-((methoxycarbonyl)amino)-5,6-dihydro-4H-cyclopenta[b]thien-2-yl)-4-oxo-1,4-dihydro-3-quinolinecarboxylic acid;
7-(5-(acetylamino)-5,6-dihydro-4H-cyclopenta[b]thien-2-yl)- 1 -cyclopropyl-8-methoxy-4-oxo-1,4-dihydro-3-quinolinecarboxylic acid;
1-cyclopropyl-8-methoxy-7-(5-(((4-methylphenyl)sulfonyl)amino)-5,6-dihydro-4H-cyclopenta[b]thien-2-yl)-4-oxo-1,4-dihydro-3-quinolinecarboxylic acid;
1-cyclopropyl-8-methoxy-7-(5-((methylsulfonyl)amino)-5,6-dihydro-4H-cyclopenta[b]thien-2-yl)-4-oxo-1,4-dihydro-3-quinolinecarboxylic acid;
7-((5 E/Z)-5-((benzyloxy)imino)-5,6-dihydro-4H-cyclopenta[b]thien-2-yl)-1-cyclopropyl-8-methoxy-4-oxo-1,4-dihydro-3-quinolinecarboxylic acid;
1-cyclopropyl-7-((5E/Z)-5-(hydroxyimino)-5,6-dihydro-4H-cyclopenta[b]thien-2-yl)-8-methoxy-4-oxo-1,4-dihydro-3-quinolinecarboxylic acid;
7-(4-amino-4-methyl-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(5-(dimethylamino)-5,6-dihydro-4H-cyclopenta[b]thien-2-yl)-8-methoxy-4-oxo- 1 ,4-dihydro-3-quinolinecarboxylic acid;
7-(4-azido-5,5-dimethyl-4,5,6,7-tetrahydro-1-benzothien-2-yl)-1-cyclopropyl-8-methoxy-4-oxo-1,4-dihydro-3-quinolinecarboxylic acid;
7-(7-amino-4,5,6,7-tetrahydro-1-benzothien-3-yl)-1-cyclopropyl-8-methoxy-4-oxo-1,4-dihydro-3-quinolinecarboxylic acid;
7-(7-amino-4,5,6,7-tetrahydro-1-benzothien-2-yl)-1-cyclopropyl-8-(difluoromethoxy)-4-oxo-1,4-dihydro-3-quinolinecarboxylic acid;
1-cyclopropyl-7-(7-hydroxy-5,5-dimethyl-4,5,6,7-tetrahydro-1-benzothien-2-yl)-8-methoxy-4-oxo-1,4-dihydro-3-quinolinecarboxylic acid;
7-(7-amino-5,5-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-hydroxy-4,5,6,7-tetrahydro-1-benzofuran-2-yl)-8-methoxy-4-oxo-1,4-dihydro-3-quinolinecarboxylic acid;
7-(4-amino-4,5,6,7-tetrahydro-1-benzofiuran-2-yl)-1-cyclopropyl-8-methoxy-4-oxo-1,4-dihydro-3-quinolinecarboxylic acid;
7-(7-hydroxy-6-spirocyclohexyl-4,5,6,7-tetrahydro- 1 -benzothien-2-yl)-1-cyclopropyl-8-methoxy-4-oxo-1,4-dihydroquinoline-3-carboxylic acid;
7-(7-amino-5,5-dimethyl-4,5,6,7-tetrahydro-1-benzothien-2-yl)-1-cyclopropyl-8-methoxy-4-oxo-1,4-dihydroquinoline-3-carboxylic acid;
7-(6-(2-aminoethyl)-4,5,6,7-tetrahydro-1-benzothien-2-yl)-1-cyclopropyl-8-methoxy-4-oxo-1,4-dihydroquinoline-3-carboxylic acid;
7-(7-amino-4,5,6,7-tetrahydro-1-benzothien-2-yl)-1-cyclopropyl-6-fluoro-4-oxo-1,4-dihydro-1,8-naphthyridine-3-carboxylic acid;
1-(2,4-difluorophenyl)-6-fluoro-7-(7-hydroxy-4,5 ,6,7-tetrahydro-1-benzothien-2-yl)-4-oxo- 1,4-dihydro-1,8-naphthyridine-3-carboxylic acid;
7-(4-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid;
1-(2,4-difluorophenyl)-6-fluoro-7-(4-hydroxy-4,5,6,7-tetrahydro-1-benzothien-2-yl)-4-oxo-1,4-dihydro-1,8-naphthyridine-3-carboxylic acid;
1-(2,4-difluorophenyl)-6-fluoro-7-(4-(methylamino)-4,5,6,7-tetrahydro-1-benzothien-2-yl)-4-oxo-1,4-dihydro-1,8-naphthyridine-3-carboxylic acid;
7-(7-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid;
1-(2,4-difluorophenyl)-6-fluoro-8-methoxy-7-(4-(methylamino)-4,5,6,7-tetrahydro-1-benzothien-2-yl)-4-oxo-1,4-dihydroquinoline-3-carboxylic acid;
1-cyclopropyl-7-(4-(methylamino)-4,5,6,7-tetrahydro-1-benzothien-2-yl)-4-oxo-1,4-dihydroquinoline-3-carboxylic acid;
1-cyclopropyl-7-(7-hydroxy-4,5,6,7-tetrahydro-1-benzothien-2-yl)-4-oxo-1,4-dihydroquinoline-3-carboxylic acid;
1-cyclopropyl-7-(4-hydroxy-4,5,6,7-tetrahydro-1-benzothien-2-yl)-4-oxo-1,4-dihydroquinoline-3-carboxylic acid;
7-(7-amino-4,5,6,7-tetrahydro-1-benzothien-2-yl)-1-cyclopropyl-4-oxo-1,4-dihydro-1,8-naphthyridine-3-carboxylic acid;
1-(2,4-difluorophenyl)-6-fluoro-7-(4-hydroxy-4,5,6,7-tetrahydro-1-benzothien-2-yl)-8-methoxy-4-oxo-1,4-dihydroquinoline-3-carboxylic acid;
1-cyclopropyl-5-fluoro-7-(4-(methylamino)-4,5,6,7-tetrahydro-1-benzothien-2-yl)-4-oxo-1,4-dihydroquinoline-3-carboxylic acid;
1-(2,4-difluorophenyl)-6-fluoro-4-oxo-7-(4,5,6,7-tetrahydro-1-benzothien-2-yl)-1,4-dihydro-1,8-naphthyridine-3-carboxylic acid;
7-(4-amino-4,5,6,7-tetrahydro-1-benzothien-2-yl)-1-cyclopropyl-4-oxo-1,4-dihydroquinoline-3-carboxylic acid;
7-(7-amino-4,5,6,7-tetrahydro-1-benzothien-2-yl)-1-cyclopropyl-4-oxo 1,4-dihydroquinoline-3-carboxylic acid;
1-cyclopropyl-5,8-difluoro-7-(4-(methylamino)-4,5,6,7-tetrahydro-1-benzothien-2-yl)-4-oxo-1,4-dihydroquinoline-3-carboxylic acid;
1-cyclopropyl-6-fluoro-7-(4-(methylamino)-4,5,6,7-tetrahydro-1-benzothien-2-yl)-4-oxo-1,4-dihydroquinoline-3-carboxylic acid;
1-cyclopropyl-6,8-difluoro-7-(4-(methylamino)-4,5,6,7-tetrahydro-1-benzothien-2-yl)-4-oxo-1,4-dihydroquinoline-3-carboxylic acid;
1-(2,4-difluorophenyl)-7-(7-hydroxy-4,5,6,7-tetrahydro-1-benzothien-2-yl)-8-methoxy-4-oxo-1,4-dihydroquinoline-3-carboxylic acid;
1-cyclopropyl-6-fluoro-7-(7-hydroxy-4,5,6,7-tetrahydro-1-benzothien-2-yl)-4-oxo-1,4-dihydroquinoline-3-carboxylic acid;
1-cyclopropyl-6,8-difluoro-7-(4-hydroxy-4,5,6,7-tetrahydro-1-benzothien-2-yl)-4-oxo-1,4-dihydroquinoline-3-carboxylic acid;
1-cyclopropyl-6,8-difluoro-7-(7-hydroxy-4,5,6,7-tetrahydro-1-benzothien-2-yl)-4-oxo-1,4-dihydroquinoline-3-carboxylic acid;
1-cyclopropyl-6-fluoro-7-(4-hydroxy-4,5,6,7-tetrahydro-1-benzothien-2-yl)-4-oxo-1,4-dihydroquinoline-3-carboxylic acid;
1-(2,4-difluorophenyl)-7-(4-hydroxy-4,5,6,7-tetrahydro-1-benzothien-2-yl)-8-methoxy-4-oxo-1,4-dihydroquinoline-3 -carboxylic acid;
1-(2,4-difluorophenyl)-6-fluoro-7-(7-hydroxy-4,5,6,7-tetrahydro-1-benzothien-2-yl)-8-methoxy-4-oxo-1,4-dihydroquinoline-3-carboxylic acid;
1-cyclopropyl-6-fluoro-7-(7-hydroxy-4,5,6,7-tetrahydro-1-benzothien-2-yl)-8-methoxy-4-oxo-1,4-dihydroquinoline-3-carboxylic acid;
1-cyclopropyl-5,8-difluoro-7-(4-hydroxy-4,5,6,7-tetrahydro-1-benzothien-2-yl)-4-oxo-1,4-dihydroquinoline-3-carboxylic acid;
1-cyclopropyl-5,8-difluoro-7-(7-hydroxy-4,5,6,7-tetrahydro-1-benzothien-2-yl)-4-oxo-1,4-dihydroquinoline-3-carboxylic acid;
1-cyclopropyl-7-(7-((4-fluorobenzyl)amino)-4,5,6,7-tetrahydro-1-benzothien-2-yl)-8-methoxy-4-oxo-1,4-dihydroquinoline-3-carboxylic acid;
1-cyclopropyl-5-fluoro-7-(4-hydroxy-4,5,6,7-tetrahydro-1-benzothien-2-yl)-4-oxo-1,4-dihydroquinoline-3-carboxylic acid;
1-(2,4-difluorophenyl)-8-methoxy-7-(4-(methylamino)-4,5,6,7-tetrahydro-1-benzothien-2-yl)-4-oxo-1,4-dihydroquinoline-3-carboxylic acid;
7-(4-amino-4,5,6,7-tetrahydro-1-benzothien-2-yl)-1-(2,4-difluorophenyl)-6-fluoro-8-methoxy-4-oxo-1,4-dihydroquinoline-3-carboxylic acid;
1-(2,4-difluorophenyl)-7-(4-(methylamino)-4,5,6,7-tetrahydro-1-benzothien-2-yl)-4-oxo-1,4-dihydro-1,8-naphthyridine-3-carboxylic acid;
7-(4-amino-4,5,6,7-tetrahydro-1-benzothien-2-yl)-1-cyclopropyl-6-fluoro-4-oxo-1,4-dihydroquinoline-3-carboxylic acid;
7-(4-amino-4,5,6,7-tetrahydro-1-benzothien-2-yl)-1-cyclopropyl-6,8-difluoro-4-oxo-1,4-dihydroquinoline-3-carboxylic acid;
7-(7-amino-4,5,6,7-tetrahydro-1-benzothien-2-yl)-1-cyclopropyl-6,8-difluoro-4-oxo-1,4-dihydroquinoline-3-carboxylic acid;
7-(7-amino-4,5,6,7-tetrahydro-1-benzothien-2-yl)-1-cyclopropyl-6-fluoro-4-oxo-1,4- dihydroquinoline-3-carboxylic acid;
1-cyclopropyl-7-(7-hydroxy-4,5,6,7-tetrahydro-1-benzothien-2-yl)-6-methyl-4-oxo-1,4-dihydroquinoline-3-carboxylic acid;
1-cyclopropyl-6-methyl-7-(4-(methylamino)-4,5,6,7-tetrahydro-1-benzothien-2-yl)-4-oxo-1,4-dihydroquinoline-3-carboxylic acid;
7-(7-amino-4,5,6,7-tetrahydro-1-benzothien-2-yl)-1-(2,4-difluorophenyl)-8-methoxy-4-oxo-1,4-dihydroquinoline-3-carboxylic acid;
8-chloro-1-cyclopropyl-7-(4-hydroxy-4,5,6,7-tetrahydro-1-benzothien-2-yl)-4-oxo-1,4-dihydroquinoline-3-carboxylic acid;
8-chloro-1-cyclopropyl-7-(7-hydroxy-4,5,6,7-tetrahydro-1-benzothien-2-yl)-4-oxo-1,4-dihydroquinoline-3-carboxylic acid;
1-cyclopropyl-6-fluoro-7-(4-hydroxy-4,5 ,6,7-tetrahydro-1-benzothien-2-yl)-8-methyl-4-oxo-1,4-dihydroquinoline-3-carboxylic acid;
1-cyclopropyl-6-fluoro-8-methyl-7-(4-(methylamino)-4,5,6,7-tetrahydro-1-benzothien-2-yl)-4-oxo-1,4-dihydroquinoline-3-carboxylic acid;
7-(4-amino-4,5,6,7-tetrahydro-1-benzothien-2-yl)- 1-(2,4-difluorophenyl)-8-methoxy-4-oxo-1,4-dihydroquinoline-3-carboxylic acid;
5-amino-1-cyclopropyl-6,8-difluoro-7-(7-hydroxy-4,5,6,7-tetrahydro-1-benzothien-2-yl)-4-oxo- 1 ,4-dihydroquinoline-3-carboxylic acid;
1-cyclopropyl-6-fluoro-7-(7-hydroxy-4,5,6,7-tetrahydro-1-benzothien-2-yl)-4-oxo-1,4-dihydro-1,8-naphthyridine-3-carboxylic acid;
7-(4-amino-4,5,6,7-tetrahydro-1-benzothien-2-yl)-1-cyclopropyl-6-fluoro-8-methyl-4-oxo-1,4-dihydroquinoline-3-carboxylic acid;
1-cyclopropyl-7-(7-hydroxy-4,5,6,7-tetrahydro-1-benzothien-2-yl)-6-methyl-4-oxo-1,4-dihydro-1,8-naphthyridine-3-carboxylic acid;
7-(4-amino-4,5,6,7-tetrahydro-1-benzothien-2-yl)-1-cyclopropyl-5,8-difluoro-4-oxo-1,4-dihydroquinoline-3-carboxylic acid;
7-(7-amino-4,5,6,7-tetrahydro-1-benzothien-2-yl)-1-cyclopropyl-5,8-difluoro-4-oxo-1,4-dihydroquinoline-3-carboxylic acid;
7-(7-amino-4,5,6,7-tetrahydro-1-benzothien-2-yl)-1-cyclopropyl-6-methyl-4-oxo-1,4-dihydroquinoline-3-carboxylic acid;
7-(4-amino-4,5,6,7-tetrahydro-1-benzothien-2-yl)-8-chloro-1-cyclopropyl-4-oxo-1,4-dihydroquinoline-3-carboxylic acid;
7-(7-amino-4,5,6,7-tetrahydro-1-benzothien-2-yl)-8-chloro-1-cyclopropyl-4-oxo-1,4-dihydroquinoline-3-carboxylic acid;
5-amino-7-(4-amino-4,5,6,7-tetrahydro-1-benzothien-2-yl)-1-cyclopropyl-6,8-difluoro-4-oxo-1,4-dihydroquinoline-3-carboxylic acid;
7-(7-amino-4,5,6,7-tetrahydro-1-benzothien-2-yl)- 1 -cyclopropyl-6-methyl-4-oxo-1,4-dihydro-1,8-naphthyridine-3-carboxylic acid;
7-(7-(benzylamino)-4,5,6,7-tetrahydro-1-benzothien-2-yl)-1-cyclopropyl-8-methoxy-4-oxo-1,4-dihydroquinoline-3-carboxylic acid;
1-cyclopropyl-8-methoxy-4-oxo-7-(7-((pyridin-3-ylmethyl)amino)-4,5,6,7-tetrahydro-1-benzothien-2-yl)-1,4-dihydroquinoline-3-carboxylic acid;
7-(4-amino-4,5,6,7-tetrahydro-1-benzothien-2-yl)-1-cyclopropyl-6-methyl-4-oxo-1,4-dihydroquinoline-3-carboxylic acid;
1-cyclopropyl-7-(4-hydroxy-4,5,6,7-tetrahydro-1-benzothien-2-yl)-6-methyl-4-oxo-1,4-dihydroquinoline-3-carboxylic acid;
7-(4-amino-4,5,6,7-tetrahydro-1-benzothien-2-yl)-1-cyclopropyl-6-methyl-4-oxo-1,4-dihydro-1,8-naphthyridine-3-carboxylic acid;
7-(7-amino-6,6-dimethyl-4,5,6,7-tetrahydro- 1 -benzothien-2-yl)-1-cyclopropyl-6-fluoro-4-oxo-1,4-dihydroquinoline-3-carboxylic acid;
1-cyclopropyl-6-fluoro-7-(7-hydroxy-6,6-dimethyl-4,5,6,7-tetrahydro-1-benzothien-2-yl)-4-oxo-1,4-dihydroquinoline-3-carboxylic acid;
7-(7-amino-6,6-dimethyl-4,5,6,7-tetrahydro-1-benzothien-2-yl)-1-cyclopropyl-6-fluoro-4-oxo-1,4-dihydro-1,8-naphthyridine-3-carboxylic acid;
1-cyclopropyl-6-fluoro-7-(7-hydroxy-6,6-dimethyl-4,5,6,7-tetrahydro-1-benzothien-2-yl)-4-oxo-1,4-dihydro-1,8-naphthyridine-3-carboxylic acid;
5-amino-7-(7-amino-4,5,6,7-tetrahydro-1-benzothien-2-yl)-1-cyclopropyl-6,8-difluoro-4-oxo-1,4-dihydroquinoline-3-carboxylic acid;
5-amino-1-cyclopropyl-6,8-difluoro-7-(4-(methylamino)-4,5,6,7-tetrahydro-1-benzothien-2-yl)-4-oxo-1,4-dihydroquinoline-3-carboxylic acid;
7-(7-amino-6,6-dimethyl-4,5,6,7-tetrahydro-1-benzothien-2-yl)-8-chloro-1-cyclopropyl-4-oxo-1,4-dihydroquinoline-3-carboxylic acid;
7-(7-amino-6,6-dimethyl-4,5,6,7-tetrahydro-1-benzothien-2-yl)-1-cyclopropyl-6-fluoro-8-methoxy-4-oxo-1,4-dihydroquinoline-3-carboxylic acid;
5-amino-7-(7-amino-4,5,6,7-tetrahydro-1-benzothien-2-yl)- 1 -cyclopropyl-8-fluoro-4-oxo-1,4-dihydroquinoline-3-carboxylic acid;
5-amino-1-cyclopropyl-8-fluoro-7-(7-hydroxy-4,5,6,7-tetrahydro-1-benzothien-2-yl)-4-oxo-1,4-dihydroquinoline-3-carboxylic acid;
5-amino-1-cyclopropyl-8-fluoro-7-(4-hydroxy-4,5,6,7-tetrahydro-1-benzothien-2-yl)-4-oxo-1,4-dihydroquinoline-3-carboxylic acid;
5-amino-1-cyclopropyl-7-(4-hydroxy-4,5,6,7-tetrahydro-1-benzothien-2-yl)-4-oxo-1,4-dihydroquinoline-3-carboxylic acid;
5-amino-7-(7-amino-4,5,6,7-tetrahydro-1-benzothien-2-yl)-6-chloro-1-cyclopropyl-8-fluoro-4-oxo-1,4-dihydroquinoline-3-carboxylic acid;
5-amino-6-chloro-1-cyclopropyl-8-fluoro-7-(7-hydroxy-4,5,6,7-tetrahydro-1-benzothien-2-yl)-4-oxo-1,4-dihydroquinoline-3-carboxylic acid;
5-amino-6-chloro-1-cyclopropyl-8-fluoro-7-(4-hydroxy-4,5,6,7-tetrahydro-1-benzothien-2-yl)-4-oxo-1,4-dihydroquinoline-3-carboxylic acid;
8-chloro-1-cyclopropyl-6-fluoro-7-(7-hydroxy-4,5,6,7-tetrahydro-1-benzothien-2-yl)-4-oxo-1,4-dihydroquinoline-3-carboxylic acid;
8-chloro-1-cyclopropyl-6-fluoro-7-(4-(methylamino)-4,5,6,7-tetrahydro-1-benzothien-2-yl)-4-oxo-1 ,4-dihydroquinoline-3-carboxylic acid;
8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid;
8-chloro-1-cyclopropyl-7-(4-(methylamino)-4,5,6,7-tetrahydro-1-benzothien-2-yl)-4-oxo-1,4-dihydroquinoline-3-carboxylic acid;
7-(7-amino-4,5,6,7-tetrahydro-1-benzothien-2-yl)-1-cyclopropyl-8-(difluoromethoxy)-6-fluoro-4-oxo-1,4-dihydroquinoline-3-carboxylic acid;
1-cyclopropyl-8-(difluoromethoxy)-6-fluoro-7-(7-hydroxy-6,6-dimethyl-4,5,6,7-tetrahydro-1-benzothien-2-yl)-4-oxo-1,4-dihydroquinoline-3-carboxylic acid;
1-cyclopropyl-6-fluoro-7-(4-hydroxy-4,5,6,7-tetrahydro-1-benzothien-2-yl)-5-methyl-4-oxo-1,4-dihydro-1,8-naphthyridine-3-carboxylic acid;
7-(4-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid;
7-(7-amino-4,5,6,7-tetrahydro-1-benzothien-2-yl)-1-cyclopropyl-6-fluoro-8-methyl-4-oxo-1,4-dihydroquinoline-3-carboxylic acid;
1-cyclopropyl-6-fluoro-7-(7-hydroxy-4,5,6,7-tetrahydro-1-benzothien-2-yl)-8-methyl-4-oxo-1,4-dihydroquinoline-3-carboxylic acid;
1-cyclopropyl-6-fluoro-7-(7-hydroxy-4,5,6,7-tetrahydro-1-benzothien-2-yl)-5-methyl-4-oxo-1,4-dihydro-1,8-naphthyridine-3-carboxylic acid;
7-(7-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid;
7-(7-amino-6,6-dimethyl-4,5,6,7-tetrahydro-1-benzothien-2-yl)-1-cyclopropyl-6-fluoro-8-methyl-4-oxo-1,4-dihydroquinoline-3-carboxylic acid; and
7-(7-amino-6,6-dimethyl-4,5,6,7-tetrahydro-1-benzothien-2-yl)-1-cyclopropyl-6,8-difluoro-4-oxo-1,4-dihydroquinoline-3-carboxylic acid.

22. A compound according to claim 21 which is
7-(7-amino-4,5,6,7-tetrahydro-1-benzothien-2-yl)-1-cyclopropyl-8-methoxy-4-oxo-1,4-dihydro-3-quinolinecarboxylic acid hydrochloride.

23. A compound selected from the group consisting of
1-cyclopropyl-4-oxo-7-(4,5,6,7-tetrahydrothieno[3,2-c]pyridin-2-yl)-1 ,4-dihydro-3-quinolinecarboxylic acid,
1-cyclopropyl-8-methoxy-4-oxo-7-(4,5,6,7-tetrahydrothieno[3,2-c]pyridin-2-yl)-1,4-dihydro-3-quinolinecarboxylic acid,
1-cyclopropyl-4-oxo-7-(4,5,6,7-tetrahydrothieno[3,2-c]pyridin-2-yl)-1,4-dihydro[1,8]naphthyridine-3-carboxylic acid,
1-cyclopropyl-6-fluoro-4-oxo-7-(4,5,6,7-tetrahydrothieno[3,2-c]pyridin-2-yl)-1,4-dihydro[1,8]naphthyridine-3-carboxylic acid,
1-eylopropyl-8-(difluoromethoxy)-4-oxo-7-(4,5,6,7-tetrahydrothieno[3,2-c]pyridin-2-yl)-1,4-dihydro-3-quinolinecarboxylic acid,
1-cyclopropyl-4-oxo-7-(4,5,6,7-tetrahydrothieno[2,3 -c]pyridin-2-yl)-1,4-dihydro-3-quinolinecarboxylic acid,
1-cyclopropyl-8-methoxy-4-oxo-7-(4,5,6,7-tetrahydrothieno[2,3-c]pyridin-2-yl)-1,4-dihydro-3-quinolinecarboxylic acid,
1-cyclopropyl-4-oxo-7-(4,5,6,7-tetrahydrothieno[2,3-c]pyridin-2-yl)- 1,4-dihydro[1,8]naphthyridine-3-carboxylic acid,
1-cyclopropyl-6-fluoro-4-oxo-7-(4,5,6,7-tetrahydrothieno[2,3 -c]pyridin-2-yl)-1,4-dihydro[1,8]naphthyridine-3-carboxylic acid,
1-cyclopropyl-8-(difluoromethoxy)-4-oxo-7-(4,5,6,7-tetrahydrothieno[2,3-c]pyridin-2-yl)-1,4-dihydro-3-quinolinecarboxylic acid,
1-cyelopropyl-7-(6-methyl-4,5,6,7-tetrahydrothieno[3,2-c]pyridin-2-yl)-4-oxo-1,4-dihydro[1,8]naphthyridine-3-carboxylic acid,
1-cyclopropyl-6-fluoro-7-(6-methyl-4,5,6,7-tetrahydrothieno[3 ,2-c]pyridin-2-yl)-4-oxo-1,4-dihydro[1,8]naphthyridine-3-carboxylic acid,
1-cyclopropyl-7-(6-methyl-4,5,6,7-tetrahydrothieno[3,2-c]pyridin-2-yl)-4-oxo-1,4-dihydro-3-quinolinecarboxylic acid,
1-cyclopropyl-8-(difluoromethoxy)-7-(6-methyl-4,5 ,6,7-tetrahydrothieno[3,2-c]pyridin-2-yl)-4-oxo-1,4-dihydro-3-quinolinecarboxylic acid,
1-cyclopropyl-8-methoxy-7-(6-methyl-4,5,6,7-tetrahydrothieno[3,2-c]pyridin-2-yl)-4-oxo-1,4-dihydro-3-quinolinecarboxylic acid,
1-cyclopropyl-7-(4-methyl-4,5,6,7-tetrahydrothieno[3 ,2-c]pyridin-2-yl)-4-oxo- 1,4-dihydro-3-quinolinecarboxylic acid,
1-cyclopropyl-7-(4-methyl-4,5,6,7-tetrahydrothieno[3,2-c]pyridin-2-yl)-4-oxo-1,4-dihydro[1,8]naphthyridine-3-carboxylic acid,
1-cyclopropyl-6-fluoro-7-(4-methyl-4,5,6,7-tetrahydrothieno[3,2-c]pyridin-2-yl)-4-oxo-1,4-dihydro[1,8]naphthyridine-3-carboxylic acid,
1-cyclopropyl-8-methoxy-7-(4-methyl-4,5 ,6,7-tetrahydrothieno[3,2-c]pyridin-2-yl)-4-oxo-1,4-dihydro-3-quinolinecarboxylic acid,
1-cyclopropyl-8-(difluoromethoxy)-7-(4-methyl-4,5,6,7-tetrahydrothieno[3,2-c]pyridin-2-yl)-4-oxo-1,4-dihydro-3-quinolinecarboxylic acid,
1-cyclopropyl-7-(7-methyl-4,5,6,7-tetrahydrothieno[2,3-c]pyridin-2-yl)-4-oxo-1,4-dihydro[1,8]naphthyridine-3-carboxylic acid,
1-cyclopropyl-6-fluoro-7-(7-methyl4,5,6,7-tetrahydrothieno[2,3-c]pyridin-2-yl)-4-oxo-1,4-dihydro[1,8]naphthyridine-3-carboxylic acid,
1-cyclopropyl-8-methoxy-7-(7-methyl-4,5,6,7-tetrahydrotheino[2,3-c]pyrindin-2-yl)-4-oxo-1,4-dihydro-3-quinolinecarboxylic acid,
1-cyclopropyl-7-(7,7-dimethyl-4,5,6,7-tetrahydrothieno[3,2-c]pyridin-2-yl)4-oxo-1,4-dihydro[1,8]naphthyridine-3-carboxylic acid,
1-cyclopropyl-7-(7,7-dimethyl-4,5,6,7-tetrahydrothieno[3,2-c]pyridin-2-yl)-4-oxo-1,4-dihydro-3-quinolinecarboxylic acid,
1-cyclopropyl-7-(7,7-dimethyl-4,5,6,7-tetrahydrothieno[3,2-c]pyridin-2-yl)-8-methoxy-4-oxo-1,4-dihydro-3-quinolinecarboxylic acid,
1-cyclopropyl-7-(7,7-dimethyl-4,5,6,7-tetrahydrothieno[3,2-c]pyridin-2-yl)-6-fluoro-4-oxo-1,4-dihydro[1,8]naphthyridine-3-carboxylic acid,
1-cyclopropyl-8-(difluoromethoxy)-7-(7,7-dimethyl-4,5,6,7-tetrahydrothieno[3,2-c]pyridin-2-yl)-4-oxo-1,4-dihydro-3-quinolinecarboxylic acid,
1-cyclopropyl-7-(4-methyl-4,5,6,7-tetrahydrot4ieno[2,3-c]pyridin-2-yl)-4-oxo-1,4-dibydro-3-quinolinecarboxylic acid,
1-cyclopropyl-8-methoxy-7-(4-methyl-4,5,6,7-tetrahydrothieno[2,3-c]pyridin-2-yl)-4-oxo-1,4-dihydro-3-quinolinecarboxylic acid,
1-cyclopropyl-8-(difluoromethoxy)-7-(4-methyl-4,5,6,7-tetrahydrothieno[2,3-c]pyridin-2-yl)-4-oxo-1,4-dihydro-3-quinolinecarboxylic acid,
1-cyclopropyl-7-(4-methyl-4,5,6,7-tetrahydrothieno[2,3 -c]pyridin-2-yl)-4-oxo-1,4-dihydro[1,8]naphthyridine-3-carboxylic acid,
1-cyclopropyl-6-fluoro-7-(4-methyl-4,5,6,7-tetrahydrothieno[2,3-c]pyridin-2-yl)-4-oxo-1,4-dihydro[1,8]naphthyridine-3 -carboxylic acid,
1-cyclopropyl-7-(4,4-dimethyl-4,5,6,7-tetrahydrothieno[2,3-c]pyridin-2-yl)-8-methoxy-4-oxo-1,4-dihydro-3-quinolinecarboxylic acid,
1-cyclopropyl-6-fluoro-7-(4-hydroxy-4,5,6,7-tetrahydro-1-benzothien-2-yl)-4-oxo-1,4-dihydro[1,8]naphthyridine-3-carboxylic acid,
1-cyclopropyl-7-(4-hydroxy-5,6-dihydro-4H-thieno[2,3 -b]thiopyran-2-yl)-8-methoxy-4-oxo-1 ,4-dihydro-3-quinolinecarboxylic acid,
7-(4-amino-5,6-dihydro-4H-thieno[2,3-b]thiopyran-2-yl)- 1 -cyclopropyl-8-methoxy-4-oxo-1,4-dihydro-3-quinolinecarboxylic acid,
1-cyclopropyl-7-(4-hydroxy-4,5,6,7-tetrahydro-1-benzothien-2-yl)-8-methoxy-4-oxo-1,4-dihydro-3-quinolinecarboxylic acid,
7-(4-azido-4,5,6,7-tetrahydro-1-benzothien-2-yl)-1-cyclopropyl-8-methoxy-4-oxo-1,4-dihydro-3-quinolinecarboxylic acid,
7-(4-amino-4,5,6,7-tetrahydro-1-benzothien-2-yl)-1-cyclopropyl-8-methoxy-4-oxo-1,4-dihydro-3-quinolinecarboxylic acid,
1-cyclopropyl-7-(7-hydroxy-4,5,6,7-tetrahydro-1-benzothien-2-yl)-8-methoxy-4-oxo-1,4-dihydro-3-quinolinecarboxylic acid,
1-cyclopropyl-7-(5-hydroxy-4,5,6,7-tetrahydro-1-benzothien-2-yl)-8-methoxy-4-oxo-1,4-dihydro-3-quinolinecarboxylic acid,
1-cyclopropyl-7-(5-(hydroxymethyl)-4,5,6,7-tetrahydro-1-benzothien-2-yl)-8-methoxy-4-oxo-1,4-dihydro-3-quinolinecarboxylic acid,
1-cyclopropyl-7-(5-(hydroxymethyl)-6,7-dihydro-1-benzothien-2-yl)-8-methoxy-4-oxo-1,4-dihydro-3-quinolinecarboxylic acid,
1-cyclopropyl-7-(5-hydroxy-5,6-dihydro-4H-cyclopenta[b]thien-2-yl)-8-methoxy-4-oxo-1,4-dihydro-3-quinolinecarboxylic acid,
1-cyclopropyl-8-methoxy-7-(4-methoxy-5,6-dihydro-4H-cyclopenta[b]thien-2-yl)-4-oxo-1,4-dihydro-3-quinolinecarboxylic acid,
1-cyclopropyl-7-(6-hydroxy-4,5,6,7-tetrahydro-1-benzothien-2-yl)-8-methoxy-4-oxo-1,4-dihydro-3-quinolinecarboxylic acid,
1-cyclopropyl-6-fluoro-4-oxo-7-(4-oxo-4,5,6,7-tetrahydro-1-benzothien-2-yl)-1,4-dihydro[1,8]naphthyridine-3-carboxylic acid,
1-cyclopropyl-6-fluoro-7-((4E/Z)-4-(hydroxyimino)-4,5,6,7-tetrahydro-1-benzothien-2-yl)-4-oxo-1,4-dihydro(1,8)naphthyridine-3-carboxylic acid;
1-cyclopropyl-8-methoxy-4-oxo-7-(4-oxo-4,5,6,7-tetrahydro-1-benzothien-2-yl)-1,4-dihydro-3-quinolinecarboxylic acid;
1-cyclopropyl-7-((4E/Z)-4-(hydroxyimino)-4,5,6,7-tetrahydro-1-benzothien-2-yl)-8-methoxy-4-oxo-1,4-dihydro-3-quinolinecarboxylic acid;
1-cyclopropyl-8-diflouromethoxy-7-(7-methyl-4,5,6,7-tetrahydrotheino(2,3-c)pyrindin-2-yl)-4-oxo- 1,4-dihydro-3-quinolinecarboxylic acid;
1-cyclopropyl-8-methoxy-4-oxo-7-(5-oxo-4,5,6,7-tetrahydro-1-benzothien-2-yl)-1,4-dihydro-3-quinolinecarboxylic acid;
7-[5-(azidomethyl)-4,5,6,7-tetrahydro-1-benzothien-2-yl]-1-cyclopropyl-8-methoxy-4-oxo-1,4-dihydro-3-quinolinecarboxylic acid;
1-cyclopropyl-7-((5E/Z)-5-(hydroxyimino)-4,5,6,7-tetrahydro-1-benzothien-2-yl)-8-methoxy-4-oxo-1,4-dihydro-3-quinolinecarboxylic acid;
1-cyclopropyl-8-methoxy-7-((5E/Z)-5-(methoxyimino)-4,5,6,7-tetrahydro-1-benzothien-2-yl)-4-oxo-1,4-dihydro-3-quinolinecarboxylic acid;
1-cyclopropyl-7-(5-methyl-4,5,6,7-tetrahydrothieno[2,3-c]pyridin-2-yl)-4-oxo-1,4-dihydro[1,8]naphthyridine-3-carboxylic acid;
7-(5-bromo-4-oxo-4,5,6,7-tetrahydro-1-benzothien-2-yl)- 1 -cyclopropyl-8-methoxy-4-oxo-1,4-dihydro-3-quinolinecarboxylic acid;
1-cyclopropyl-8-methoxy-4-oxo-7-(6-oxo-4,5,6,7-tetrahydro-1-benzothien-2-yl)- 1,4-dihydro-3-quinolinecarboxylic acid;
1-cyclopropyl-7-((6E/Z)-6-(hydroxyimino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(7-oxo-4,5,6,7-tetrahydro-1-benzothien-2-yl)-1,4-dihydro-3-quinolinecarboxylic acid;
7-(5-azido-4-oxo-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((7E/Z)-7-(methoxyimino)-4,5,6,7-tetrahydro-1-benzothien-2-yl)-4-oxo-1,4-dihydro-3-quinolinecarboxylic acid;
1-cyclopropyl-8-methoxy-7-((4E/Z)-4-(methoxyimino)-4,5,6,7-tetrahydro-1-benzothien-2-yl)-4-oxo-1,4-dihydro-3-quinolinecarboxylic acid;
1-cyclopropyl-7-((4E/Z)-4-(ethoxyimino)-4,5,6,7-tetrahydro-1-benzothien-2-yl)-8-methoxy-4-oxo- 1 ,4-dihydro-3-quinolinecarboxylic acid;
1-cyclopropyl-8-methoxy-7-((6E/Z)-6-(methoxyimino)-4,5,6,7-tetrahydro-1-benzothien-2-yl)-4-oxo-1,4-dihydro-3-quinolinecarboxylic acid;
1-cyclopropyl-7-(6,7-dihydro-1-benzothien-2-yl)-8-methoxy-4-oxo-1,4-dihydro-3-quinolinecarboxylic acid;
1-cyclopropyl-8-methoxy-7-((4E/Z)-4-(4-morpholinylimino)-4,5,6,7-tetrahydro-1-benzothien-2-yl)-4-oxo-1,4-dihydro-3-quinolinecarboxylic acid;
1-cyclopropyl-7-(4,5-dihydroxy-4,5,6,7-tetrahydro-1-benzothien-2-yl)-8-methoxy-4-oxo-1,4-dihydro-3-quinolinecarboxylic acid;
7-(5-(aminomethyl)-4,5,6,7-tetrahydro-1-benzothien-2-yl)-1-cyclopropyl-8-methoxy-4-oxo-1,4-dihydro-3-quinolinecarboxylic acid;
7-(6-amino-4,5,6,7-tetrahydro-1-benzothien-2-yl)-1-cyclopropyl-8-methoxy-4-oxo-1,4-dihydro-3-quinolinecarboxylic acid;
7-((4E/Z)-4-(tert-butoxyimino)-4,5,6,7-tetrahydro-1-benzothien-2-yl)-1-cyclopropyl-8-methoxy-4-oxo-1,4-dihydro-3-quinolinecarboxylic acid;
7-((4E/Z)-4-((benzyloxy)imino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-((4E/Z)-4-(1-pyrrolidinylimino)-4,5,6,7-tetrahydro-1-benzothien-2-yl)-1,4-dihydro-3-quinolinecarboxylic acid;
7-(4-amino-4,5,6,7-tetrahydro-1-benzothien-3-yl)-1-cyclopropyl-8-methoxy-4-oxo-1,4-dihydro-3-quinolinecarboxylic acid;
7-(5-amino-4,5,6,7-tetrahydro- 1 -benzothien-2-yl)-1-cyclopropyl-8-methoxy-4-oxo-1,4-dihydro-3-quinolinecarboxylic acid;
1-cyclopropyl-8-methoxy-7-[4-(methylamino)-4,5,6,7-tetrahydro-1-benzothien-2-yl]-4-oxo-1,4-dihydro-3-quinolinecarboxylic acid;
1-cyclopropyl-7-((5E/Z)-5-(ethoxyimino)-4,5,6,7-tetrahydro-1-benzothien-2-yl)-8-methoxy-4-oxo-1,4-dihydro-3-quinolinecarboxylic acid;
7-((5E/Z)-5-((benzyloxy)imino)-4,5,6,7-tetrahydro-1-benzothien-2-yl)-1-cyclopropyl-8-methoxy-4-oxo-1,4-dihydro-3-quinolinecarboxylic acid;
7-((4E/Z)-4-((aminocarbonyl)hydrazono)-4,5,6,7-tetrahydro-1-benzothien-2-yl) 1-cyclopropyl-8-methoxy-4-oxo-1,4-dihydro-3-quinolinecarboxylic acid;
ethyl 1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylate;
ethyl 1-cyclopropyl-8-methoxy-7-((4E/Z)-4-(((2R)-2-(methoxymethyl)pyrrolidinyl)imino)-4,5,6,7-tetrahydro-1-benzothien-2-yl)-4-oxo-1,4-dihydro-3-quinolinecarboxylate;
1 -cyclopropyl-7-(4-(dimethylamino)-4,5,6,7-tetrahydro-1-benzothien-2-yl)-8-methoxy-4-oxo-1,4-dihydro-3-quinolinecarboxylic acid;
7-((4E/Z)-4-[(aminocarbothioyl)hydrazono)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((4E/Z)-4-(((methylamino)carbothioyl)hydrazono)-4,5,6,7-tetrahydro-1-benzothien-2-yl)-4-oxo-1,4-dihydro-3-quinolinecarboxylic acid;
1-cyclopropyl-8-methoxy-7-(5-methylene-4-oxo-4,5,6,7-tetrahydro-1-benzothien-2-yl)-4-oxo-1,4-dihydro-3-quinolinecarboxylic acid;
ethyl 1-cyclopropyl-8-methoxy-7-(5-methylene-4-oxo-4,5,6,7-tetrahydro-1-benzothien-2-yl)-4-oxo-1,4-dihydro-3-quinolinecarboxylate;
1-cyclopropyl-8-methoxy-7-(4-((methylsulfonyl)amino)-4,5,6,7-tetrahydro-1-benzothien-2-yl)-4-oxo-1,4-dihydro-3-quinolinecarboxylic acid;
1-cyclopropyl-8-methoxy-4-oxo-7-(4-(1H-pyrrol-1-yl)-4,5,6,7-tetrahydro-1-benzothien-2-yl)-1,4-dihydro-3-quinolinecarboxylic acid;
1-cyclopropyl-7-((4E/Z)-4-(((ethylamino)carbothioyl)hydrazono)-4,5,6,7-tetrahydro-1-benzothien-2-yl)-8-methoxy-4-oxo-1,4-dihydro-3-quinolinecarboxylic acid;
7-((4E/Z)-4-((amino(oxo)acetyl)hydrazono)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-((4E/Z)-4-((4-methyl-1-piperazinyl)imino)-4,5,6,7-tetrahydro-1-benzothien-2-yl)-4-oxo-1,4-dihydro-3-quinolinecarboxylic acid;
7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((3-pyridinylmethyl)amino)-4,5,6,7-tetrahydro-1-benzothien-2-yl)-1,4-dihydro-3-quinolinecarboxylic acid;
ethyl 7-(4-((tert-butyl(dimethyl)silyl)oxy)-4,5,6,7-tetrahydro-1-benzothien-2-yl)-1-cyclopropyl-8-methoxy-4-oxo-1,4-dihydro-3-quinolinecarboxylate;
7-((4E/Z)-4-(acetylhydrazono)-4,5,6,7-tetrahydro-1-benzothien-2-yl)-1-cyclopropyl-8-methoxy-4-oxo-1,4-dihydro-3-quinolinecarboxylic acid;
7-(4-(benzylamino)-4,5,6,7-tetrahydro-1-benzothien-2-yl)-1-cyclopropyl-8-methoxy-4-oxo- 1,4-dihydro-3 -quinolinecarboxylic acid;
1-cyclopropyl-7-(4-(ethylamino)-4,5,6,7-tetrahydro-1-benzothien-2-yl)-8-methoxy-4-oxo-1,4-dihydro-3-quinolinecarboxylic acid;
1-cyclopropyl-8-(difluoromethoxy)-7-(4-hydroxy-4,5,6,7-tetrahydro-1-benzothien-2-yl)-4-oxo-1,⁴-dihydro-3-quinolinecarboxylic acid;
1-cyclopropyl-7-((4E/Z)-4-(4,5-dihydro-1H-imidazol-2-ylhydrazono)-4,5,6,7-tetrahydro-1-benzothien-2-yl)-8-methoxy-4-oxo-1,4-dihydro-3-quinolinecarboxylic acid;
7-(4-amino-4,5,6,7-tetrahydro-1-benzothien-2-yl)-1-cyclopropyl-8-(difluoromethoxy)-4-oxo-1,4-dihydro-3-quinolinecarboxylic acid;
1-cyclopropyl-7-(4-hydroxy-2-methyl-1,1-dioxido-3,4-dihydro-2H-thieno[3,2-e][1,2]thiazin-6-yl)-8-methoxy-4-oxo-1,4-dihydro-3-quinolinecarboxylic acid;
ethyl 1-cyclopropyl-8-methoxy-7-(5 -methyl-4-oxo-4,5,6,7-tetrahydro-1-benzothien-2-yl)-4-oxo-1,4-dihydro-3-quinolinecarboxylate;
1-cyclopropyl-7-(4-((3-fluorobenzyl)amino)-4,5,6,7-tetrahydro-1-benzothien-2-yl)-8-methoxy-4-oxo-1,4-dihydro-3-quinolinecarboxylic acid;
7-((4E/Z)-4-[(aminocarbothioyl)(methyl)hydrazono)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-(S-methyl-4-oxo-4,5,6,7-tetrahydro-1-benzothien-2-yl)-4-oxo-1,4-dihydro-3-quinolinecarboxylic acid;
1-cyclopropyl-8-(difluoromethoxy)-7-(4-(methylamino)-4,5,6,7-tetrahydro-1-benzothien-2-yl)-4-oxo-1,4-dihydro-3-quinolinecarboxylic acid;
1-cyclopropyl-6-fluoro-7-(4-(methylamino)-4,5,6,7-tetrahydro-1-benzothien-2-yl)-4-oxo-1,4-dihydro[1,8]naphthyridine-3-carboxylic acid;
7-(4-amino-2-methyl-1,1-dioxido-3,4-dihydro-2H-thieno[3,2-e][1,2]thiazin-6-yl)-1-cyclopropyl-8-methoxy-4-oxo-1,4-dihydro-3-quinolinecarboxylic acid;
1-cyclopropyl-7-(4-(hydroxymethyl)-4,5,6,7-tetrahydro-1-benzothien-2-yl)-8-methoxy-4-oxo-1,4-dihydro-3-quinolinecarboxylic acid;
1-cyclopropyl-8-metoxy-4-oxo-7-(4-(1-pyrrolidinylmethyl)-4,5,6,7-tetrahydro-1-benzothien-2-yl)-1,4-dihydro-3-quinolinecarboxylic acid;
1-cyclopropyl-8-methoxy-4-oxo-7-(4-((2-pyrrolidinylmethyl)amino)-4,5,6,7-tetrahydro-1-benzothien-2-yl)-1,4-dihydro-3-quinolinecarboxylic acid;
7-(4-(acetylamino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-(propionylamino)-4,5,6,7-tetrahydro-1-benzothien-2-yl)-1,4-dihydro-3-quinolinecarboxylic acid;
1-cyclopropyl-8-methoxy-7-(4-((methoxyacetyl)amino)-4,5,6,7-tetrahydro-1-benzothien-2-yl)-4-oxo-1,4-dihydro-3-quinolinecarboxylic acid;
1-cyclopropyl-8-methoxy-4-oxo-7-(4-((tetrahydrofuranyl-2-carbonyl)amino)-4,5,6,7-tetrahydro-1-benzothien-2-yl)-1,4-dihydro-3-quinolinecarboxylic acid;
1-cyclopropyl-8-methoxy-4-oxo-7-(4-((tetrahydrofuranyl-3-carbonyl)amino)-4,5,6,7-tetrahydro-1-benzothien-2-yl)-1,4-dihydro-3-quinolinecarboxylic acid;
1-cyclopropyl-8-methoxy-7-(4-((4-morpholinylacetyl)amino)-4,5,6,7-tetrahydro-1-benzothien-2-yl)-4-oxo-1,4-dihydro-3-quinolinecarboxylic acid;
1-cyclopropyl-8-methoxy-7-(4-((3-(4-morpholinyl)propanoyl)amino)-4,5,6,7-tetrahydro- 1 -benzothien-2-yl)-4-oxo-1,4-dihydro-3-quinolinecarboxylic acid;
1-cyclopropyl-8-methoxy-4-oxo-7-(4-((1H-pyrrol-2-ylcarbonyl)amino)-4,5,6,7-tetrahydro- 1 -benzothien-2-yl)-1,4-dihydro-3-quinolinecarboxylic acid;
1-cyclopropyl-8-methoxy-4-oxo-7-(4-((3-pyridinylacetyl)amino)-4,5,6,7-tetrahydro-1-benzothien-2-yl)-1,4-dihydro-3-quinolinecarboxylic acid;
1-cyclopropyl-8-methoxy-4-oxo-7-(4-((3-pyridazinylcarbonyl)amino)-4,5,6,7-tetrahydro- 1 -benzothien-2-yl)-1,4-dihydro-3-quinolinecarboxylic acid;
1-cyclopropyl-7-(4-((1H-imidazol-2-ylcarbonyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((1,3-thiazol-2-ylcarbonyl)amino)-4,5,6,7-tetrahydro- 1 -benzothien-2-yl)-1,4-dihydro-3-quinolinecarboxylic acid;
1-cyclopropyl-8-methoxy-7-(5-(methylamino)-4,5,6,7-tetrahydro-1-benzothien-2-yl)-4-oxo-1,4-dihydro-3-quinolinecarboxylic acid;
1-cyclopropyl-8-methoxy-7-(4-(4-morpholinylmethyl)-4,5,6,7-tetrahydro-1-benzothien-2-yl)-4-oxo-1,4-dihydro-3-quinolinecarboxylic acid;
1-cyclopropyl-7-(4-((dimethylamino)methyl)-4,5,6,7-tetrahydro-1-benzothien-2-yl)-8-methoxy-4-oxo-1,4-dihydro-3-quinolinecarboxylic acid;
1-cyclopropyl-7-(4-(((dimethylamino)acetyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((2-pyridinylacetyl)amino)-4,5,6,7-tetrahydro-1-benzothien-2-yl)-1,4-dihydro-3-quinolinecarboxylic acid;
7-(4-(aminomethyl)-4,5,6,7-tetrahydro-1-benzothien-2-yl)-1-cyclopropyl-8-methoxy-4-oxo- 1,4-dihydro-3-quinolinecarboxylic acid;
1-cyclopropyl-8-methoxy-4-oxo-7-((4E/Z)-4-((4-pyridinylmethoxy)imino)-4,5,6,7-tetrahydro-1-benzothien-2-yl)-1,4-dihydro-3-quinolinecarboxylic acid;
7-(4-((2-aminoethyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-(4-((1-methyl-4-piperidinyl)amino)-4,5,6,7-tetrahydro-1-benzothien-2-yl)-4-oxo-1,4-dihydro-3-quinolinecarboxylic acid;
1-cyclopropyl-7-((4E/Z)-4-(hydroxyimino)-5-methyl-4,5,6,7-tetrahydro-1-benzothien-2-yl)-8-methoxy-4-oxo-1,4-dihydro-3-quinolinecarboxylic acid;
1-cyclopropyl-7-(4-hydroxy-7,7-dioxido-5,6-dihydro-4H-thieno[2,3-bthiopyran-2-yl)-8-methoxy-4-oxo-1,4-dihydro-3-quinolinecarboxylic acid;
7-(4-(((5-chloro-1,3-dimethyl-1H-pyrazol-4-yl)sulfonyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((4-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((phenylsulfonyl)amino)-4,5,6,7-tetrahydro-1-benzothien-2-yl)-1,4-dihydro-3-quinolinecarboxylic acid;
7-(4-(((2-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-7-(4-(((4-methoxyphenyl)sulfonyl)amino)-4,5,6,7-tetrahydro-1-benzothien-2-yl)-4-oxo-1,4-dihydro-3-quinolinecarboxylic acid;
1-cyclopropyl-8-methoxy-7-(4-(((3-nitrophenyl)sulfonyl)amino)-4,5,6,7-tetrahydro-1benzothien-2-yl)-4-oxo-1,4-dihydro-3-quinolinecarboxylic acid;
1-cyclopropyl-8-methoxy-4-oxo-7-(5-((2-pyrrolidinylmethyl)amino)-4,5,6,7-tetrahydro- 1 -benzothien-2-yl)-1,4-dihydro-3-quinolinecarboxylic acid;
7-(4-amino-7,7-dioxido-5,6-dihydro-4H-thieno[2,3-b]thiopyran-2-yl)-1-cyclopropyl-8-methoxy-4-oxo-1,4-dihydro-3-quinolinecarboxylic acid;
1-cyclopropyl-7-(4-(((3,5-dimethyl-4-isoxazolyl)sulfonyl)amino)-4,5,6,7-tetrahydro-1-benzothien-2-yl)-8-methoxy-4-oxo-1,4-dihydro-3-quinolinecarboxylic acid;
7-(4-((2,1,3-benzoxadiazol-4-ylsulfonyl)amino)-4,5,6,7-tetrahydro-1-benzothien-2-yl)-1-cyclopropyl-8-methoxy-4-oxo-1,4-dihydro-3 -quinolinecarboxylic acid;
1-cyclopropyl-7-(4-(((dimethylamino)sulfonyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((2-thienylsulfonyl)amino)-4,5,6,7-tetrahydro-1-benzothien-2-yl)-1,4-dihydro-3-quinolinecarboxylic acid;
7-(4-(((3-cyanophenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((4-(acetylamino)phenyl)sulfonyl)amino)-4,5,6,7-tetrahydro-1-benzothien-2-yl)-1-cyclopropyl-8-methoxy-4-oxo-1,4-dihydro-3-quinolinecarboxylic acid;
7-(4-((2,1,3-benzothiadiazol-4-ylsulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-(((5-(3-isoxazolyl)-2-thienyl)sulfonyl)amino)-4,5,6,7-tetrahydro-1-benzothien-2-yl)-8-methoxy-4-oxo-1,4-dihydro-3-quinolinecarboxylic acid;
1-cyclopropyl-7-(4-(((4-fluorophenyl)sulfonyl)amino)-4,5,6,7-tetrahydro-1-benzothien-2-yl)-8-methoxy-4-oxo-1,4-dihydro-3-quinolinecarboxylic acid;
7-(4-(((6-chloroimidazo[2,1-b][1,3]thiazol-5-yl)sulfonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((4-pyridinylacetyl)amino)-4,5,6,7-tetrahydro-1-benzothien-2-yl)-1,4-dihydro-3-quinolinecarboxylic acid;
1-cyclopropyl-7-(4-((2-hydroxyethyl)amino)-4,5,6,7-tetrahydro-1-benzothien-2-yl)-8-methoxy-4-oxo-1,4-dihydro-3-quinolinecarboxylic acid;
7-(4-amino-4,5,6,7-tetrahydro-1-benzothien-2-yl)-1-cyclopropyl-6-fluoro-4-oxo-1,4-dihydro[1,8]naphthyridine-3-carboxylic acid;
7-(4-((glycyl)amino)-4,5,6,7-tetrahydro-1-benzothien-2-yl)-1-cyclopropyl-8-methoxy-4-oxo-1,4-dihydro-3-quinolinecarboxylic acid;
7-(4-((D-alanyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-((D-prolyl)amino)-4,5,6,7-tetrahydro-1-benzothien-2-yl)-1,4-dihydro-3-quinolinecarboxylic acid;
7-(4-(((2R)-2-amino-3-(1H-imidazol-5-yl)propanoyl)amino)-4,5,6,7-tetrahydro-1-benzothien-2-yl)-1-cyclopropyl-8-methoxy-4-oxo-1,4-dihydro-3-quinolinecarboxylic acid;
7-(4-((leucyl)amino)-4,5,6,7-tetrahydro-1-benzothien-2-yl)-1-cyclopropyl-8-methoxy-4-oxo-1,4-dihydro-3-quinolinecarboxylic acid;
7-(4-((D-tyrosyl)amino)-4,5,6,7-tetrahydro-1-benzothien-2-yl)-1-cyclopropyl-8-methoxy-4-oxo-1,4-dihydro-3-quinolinecarboxylic acid;
7-(4-((O-methyl-D-tyrosyl)amino)-4,5,6,7-tetrahydro-1-benzothien-2-yl)-1-cyclopropyl-8-methoxy-4-oxo-1,4-dihydro-3-quinolinecarboxylic acid;
7-(4-((D-methionyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((2R)-2-amino-3-(3-pyridinyl)propanoyl)amino)-4,5,6,7-tetrahydro-1-benzothien-2-yl)-1-cyclopropyl-8-methoxy-4-oxo-1,4-dihydro-3-quinolinecarboxylic acid;
1-eylopropyl-8-methoxy-4-oxo-7-(4-(((2R)-piperidinylcarbonyl)amino)-4,5,6,7-tetrahydro-1-benzothien-2-yl)-1,4-dihydro-3-quinolinecarboxylic acid;
1-cyclopropyl-8-methoxy-4-oxo-7-(4-((4-pyrimidinylcarbonyl)amino)-4,5,6,7-tetrahydro- 1 -benzothien-2-yl)- 1,4-dihydro-3-quinolinecarboxylic acid;
1-cyclopropyl-8-methoxy-4-oxo-7-(4-((1,3-thiazol-2-ylcarbonyl)amino)-4,5,6,7-tetrahydro- 1 -benzothien-2-yl)-1,4-dihydro-3-quinolinecarboxylic acid;
1-cyclopropyl-8-methoxy-4-oxo-7-(4-((phenylacetyl)amino)-4,5,6,7-tetrahydro-1-benzothien-2-yl)-I,4-dihydro-3-quinolinecarboxylic acid;
1-cyclopropyl-7-(4-(3-furoylamino)-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4-((2-pyridinylcarbonyl)amino)-4,5,6,7-tetrahydro- 1 -benzothien-2-yl)-1,4-dihydro-3 -quinolinecarboxylic acid;
1-cyclopropyl-8-methoxy-4-oxo-7-(4-((1H-pyrazol-4-ylcarbonyl)amino)-4,5,6,7-tetrahydro- 1 -benzothien-2-yl)-1,4-dihydro-3-quinolinecarboxylic acid;
7-(4-((D-aspartyl)amino)-4,5 ,6,7-tetrahydro-1-benzothien-2-yl)-1-cyclopropyl-8-methoxy-4-oxo-1,4-dihydro-3-quinolinecarboxylic acid;
1-cylopropyl-8-methoxy-7-((4R)-4-((N-methyl-D-leucyl)amino)-4,5,6,7-tetrahydro-1-benzothien-2-yl)-4-oxo-1,4-dihydro-3-quinolinecarboxylic acid;
7-((4R)-4-((D-norleucyl)amino)-4,5,6,7-tetrahydro-1-benzothien-2-yl)-1-cyclopropyl-8-methoxy-4-oxo-1,4-dihydro-3-quinolinecarboxylic acid;
methyl 1-cyclopropyl-8-methoxy-4-oxo-7-(4-((4-pyrimidinylcarbonyl)amino)-4,5,6,7-tetrahydro-1-benzothien-2-yl)-1,4-dihydro-3-quinolinecarboxylate;
methyl 1-cyclopropyl-8-methoxy-4-oxo-7-(4-((1,3-thiazol-2-ylcarbonyl)amino)-4,5,6,7-tetrahydro-1-benzothien-2-yl)-1,4-dihydro-3-quinolinecarboxylate;
methyl 1-cyclopropyl-8-methoxy-4-oxo-7-(4-((2-pyridinylcarbonyl)amino)-4,5,6,7-tetrahydro-1-benzothien-2-yl)-1,4-dihydro-3-quinolinecarboxylate;
7-(4-((β-Omethyl-D-aspartyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-((4E)-4-((3-pyridinylmethoxy)imino)-4,5,6,7-tetrahydro- 1 -benzothien-2-yl)-1,4-dihydro-3-quinolinecarboxylic acid;
1-cyclopropyl-8-methoxy-4-oxo-7-((4E)-4-((2-pyridinylmethoxy)imino)-4,5,6,7-tetrahydro-l-benzothien-2-yl)-1,4-dihydro-3-quinolinecarboxylic acid;
ethyl 7-(4-((tert-butyl(dimethyl)silyl)oxy)-5-methyl-4,5,6,7-tetrahydro-1-benzothien-2-yl)-1-cyclopropyl-8-methoxy-4-oxo-1,4-dihydro-3-quinolinecarboxylate;
1-cyclopropyl-7-(4-hydroxy-5-methyl-4,5,6,7-tetrahydro-1-benzothien-2-yl)-8-methoxy-4-oxo-1,4-dihydro-3-quinolinecarboxylic acid;
1-cyclopropyl-8-methoxy-7-(4-(((methylanilino)carbonyl)amino)-4,5,6,7-tetrahydro-1-benzothien-2-yl)-4-oxo-1,4-dihydro-3-quinolinecarboxylic acid;
1-cyclopropyl-7-(4-(((diethylamino)carbonyl)amino)-4,5,6,7-tetrahydro-1-benzothien-2-yl)-8-methoxy-4-oxo-1,4-dihydro-3-quinolinecarboxylic acid;
1-cyclopropyl-7-(4-(((diisopropylamino)carbonyl)amino)-4,5,6,7-tetrahydro-1-benzothien-2-yl)-8-methoxy-4-oxo-1,4-dihydro-3-quinolinecarboxylic acid;
1-cyclopropyl-8-methoxy-7-(4-((4-morpholinylcarbonyl)amino)-4,5,6,7-tetrahydro-1-benzothien-2-yl)-4-oxo-1,4-dihydro-3-quinolinecarboxylic acid;
1-cyclopropyl-8-methoxy-7-(4-((methoxycarbonyl)amino)-4,5,6,7-tetrahydro-1-benzothien-2-yl)-4-oxo-1,4-dihydro-3-quinolinecarboxylic acid;
7-(4-(((benzyloxy)carbonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-((isobutoxycarbonyl)amino)-4,5,6,7-tetrahydro-1-benzothien-2-yl)-8-methoxy-4-oxo-1,4-dihydro-3-quinolinecarboxylic acid;
1-cyclopropyl-7-(4-((ethoxycarbonyl)amino)-4,5,6,7-tetrahydro-1-benzothien-2-yl)-8-methoxy-4-oxo-1,4-dihydro-3-quinolinecarboxylic acid;
7-(4-((butoxycarbonyl)amino)-4,5,6,7-tetrahydro-1-benzothien-2-yl)-1-cyclopropyl-8-methoxy-4-oxo-1,4-dihydro-3-quinolinecarboxylic acid;
7-(4-(((4-chlorobutoxy)carbonyl)amino)-4,5,6,7-tetrahydro-1-benzothien-2-yl)-1-cyclopropyl-8-methoxy-4-oxo-1,4-dihydro-3-quinolinecarboxylic acid;
1-cyclopropyl-7-((5E/Z)-5-(hydroxymethylene)-4-oxo-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-((4E/Z)-4-((3 -pyridinylmethoxy)imino)-4,5,6,7-tetrahydro- 1 -benzothien-2-yl)-1,4-dihydro-3-quinolinecarboxylic acid;
1-cyclopropyl-7-(4-(3-hydroxy-1-azetidinyl)-4,5,6,7-tetrahydro-1-benzothien-2-yl)-8-methoxy-4-oxo-1,4-dihydro-3-quinolinecarboxylic acid;
7-(4-amino-5-methyl-4,5,6,7-tetrahydro-1-benzothien-2-yl)-1-cyclopropyl-8-methoxy-4-oxo-1,4-dihydro-3-quinolinecarboxylic acid;
7-(4-amino-4,5,6,7-tetrahydro-1-benzothien-2-yl)-1-cyclopropyl-4-oxo-1,4-dihydro[1,8]naphthyridine-3-carboxylic acid;
7-(7-amino-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(7-hydroxy-4,5,6,7-tetrahydrothieno[3,2-c]pyridin-2-yl)-8-methoxy-4-oxo-1,4-dihydro-3-quinolinecarboxylic acid;
1-cyclopropyl-8-methoxy-4-oxo-7-(7-oxo-4,5,6,7-tetrahydrothieno[3,2-c]pyridin-2-yl)-1,4-dihydro-3-quinolinecarboxylic acid;
1-cyclopropyl-7-(5,5-difluoro-4-hydroxy-4,5,6,7-tetrahydro-1-benzothien-2-yl)-8-methoxy-4-oxo-1,4-dihydro-3-quinolinecarboxylic acid;
ethyl 7-(4-azido-5-methyl-4,5,6,7-tetrahydro-1-benzothien-2-yl)-1-cyclopropyl-8-methoxy-4-oxo-1,4-dihydro-3 -quinolinecarboxylate;
7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(5-(hydroxymethyl)-5-methyl-4-oxo-4,5,6,7-tetrahydro-1-benzothien-2-yl)-8-methoxy-4-oxo-1,4-dihydro-3-quinolinecarboxylic acid;
7-(7-amino-4,5,6,7-tetrahydrothieno[3,2-c]pyridin-2-yl)-1-cyclopropyl-8-methoxy-4-oxo-1,4-dihydro-3-quinolinecarboxylic acid;
1-cyclopropyl-7-(4-hydroxy-4,5,6,7-tetrahydrothieno[2,3-c]pyridin-2-yl)-8-methoxy-4-oxo-1,4-dihydro-3-quinolinecarboxylic acid;
1-cyclopropyl-6-fluoro-8-methoxy-7-(4-(methylamino)-4,5,6,7-tetrahydro-1-benzothien-2-yl)-4-oxo-1,4-dihydro-3-quinolinecarboxylic acid hydrochloride;
1-cyclopropyl-8-methoxy-4-oxo-7-(4-oxo-4,5,6,7-tetrahydrothieno[2,3-c]pyridin-2-yl)-1,4-dihydro-3-quinolinecarboxylic acid;
7-[4-(3-amino-1-azetidinyl)-4,5,6,7-tetrahydro-1-benzothien-2-yl]-1-cyclopropyl-8-methoxy-4-oxo-1,4-dihydro-3-quinolinecarboxylic acid;
1-cyclopropyl-7-(4-hydroxy-5,5-dimethyl-4,5,6,7-tetrahydro-1-benzothien-2-yl)-8-methoxy-4-oxo-1,4-dihydro-3-quinolinecarboxylic acid;
7-(4-((tert-butoxycarbonyl)amino)-5-methyl-4,5,6,7-tetrahydro-1-benzothien-2-yl)-1-cyclopropyl-8-methoxy-4-oxo-1,4-dihydro-3-quinolinecarboxylic acid;
methyl 7-[4-(acetyloxy)-4,5,6,7-tetrahydro-1-benzothien-2-yl]-1-cyclopropyl-8-methoxy-4-oxo-1,4-dihydro-3-quinolinecarboxylate;
1-cyclopropyl-8-methoxy-7-(7-(methylamino)-4,5,6,7-tetrahydro-1-benzothien-2-yl)-4-oxo-1,4-dihydro-3-quinolinecarboxylic acid;
methyl 1 -cyclopropyl-6-fluoro-7-(4-hydroxy-4,5,6,7-tetrahydro-1-benzothien-2-yl)-8-methoxy-4-oxo-1,4-dihydro-3-quinolinecarboxylate;
1-cyclopropyl-7-(7-hydroxy-4,4-dimethyl-4,5,6,7-tetrahydro-1-benzothien-2-yl)-8-methoxy-4-oxo-1,4-dihydro-3-quinolinecarboxylic acid;
methyl 7-(4-azido-4,5,6,7-tetrahydro-1-benzothien-2-yl)-1-cyclopropyl-6-fluoro-8-methoxy-4-oxo-1,4-dihydro-3-quinolinecarboxylate;
methyl 7-(4-((tert-butoxycarbonyl)amino)-4,5,6,7-tetrahydro-1-benzothien-2-yl)-1-cyclopropyl-6-fluoro-8-methoxy-4-oxo-1,4-dihydro-3-quinolinecarboxylate;
7-(4-((tert-butoxycarbonyl)amino)-4,5,6,7-tetrahydro-1-benzothien-2-yl)-1-cyclopropyl-6-fluoro-8-methoxy-4-oxo-1,4-dihydro-3-quinolinecarboxylic acid;
7-(7-amino-4,4-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(4-oxo-5-spiro-3′-(N-benzylpyrrolidine)-4,5,6,7-tetrahydro-1-benzothien-2-yl)-1,4-dihydro-3-quinolinecarboxylic acid;
7-(4-amino-4,5,6,7-tetrahydro-1-benzothien-2-yl)- 1 -cyclopropyl-6-fluoro-8-methoxy-4-oxo-1,4-dihydro-3-quinolinecarboxylic acid;
7-(4-amino-4,5 ,6,7-tetrahydrothieno[2,3-c]pyridin-2-yl)-1-cyclopropyl-8-methoxy-4-oxo-1,4-dihydro-3-quinolinecarboxylic acid;
7-(7-azido-4,5,6,7-tetrahydrothieno[3,2-c]pyridin-2-yl)-1-cyclopropyl-8-methoxy-4-oxo-1,4-dihydro-3-quinolinecarboxylic acid;
1-cyclopropyl-7-(4,4-dimethyl-4,5,6,7-tetrahydro-1-benzothien-2-yl)-8-methoxy-4-oxo-1,4-dihydro-3-quinolinecarboxylic acid;
7-(7-amino-4,5,6,7-tetrahydro-1-benzothien-2-yl)-1-cyclopropyl-6-fluoro-8-methoxy-4-oxo-1,4-dihydro-3-quinolinecarboxylic acid;
7-(5-acetyl-7-amino-4,5,6,7-tetrahydrothieno[3,2-c]pyridin-2-yl)-1-cyclopropyl-8-methoxy-4-oxo-1,4-dihydro-3-quinolinecarboxylic acid;
7-(7-amino-5-(methylsulfonyl)-4,5,6,7-tetrahydrothieno[3,2-c]pyridin-2-yl)-1-cyclopropyl-8-methoxy-4-oxo-1,4-dihydro-3-quinolinecarboxylic acid;
7-(4-amino-5,5-difluoro-4,5,6,7-tetrahydro-1-benzothien-2-yl)-1-cyclopropyl-8-methoxy-4-oxo-1,4-dihydro-3-quinolinecarboxylic acid;
7-(4-amino-3-methyl-4,5,6,7-tetrahydro-1-benzothien-2-yl)-1-cyclopropyl-8-methoxy-4-oxo-1,4-dihydro-3-quinolinecarboxylic acid;
1-cyclopropyl-7-((4E/Z)-4-(hydroxyimino)-5,5-dimethyl-4,5,6,7-tetrahydro-1-benzothien-2-yl)-8-methoxy-4-oxo-1,4-dihydro-3-quinolinecarboxylic acid;
1-cyclopropyl-7-(4-hydroxy-6,6-dimethyl-4,5,6,7-tetrahydro-1-benzothien-2-yl)-8-methoxy-4-oxo-1,4-dihydro-3-quinolinecarboxylic acid;
7-(7-amino-6-methyl-4,5 ,6,7-tetrahydro-1-benzothien-2-yl)-1-cyclopropyl-8-methoxy-4-oxo-1,4-dihydro-3-quinolinecarboxylic acid hydrochloride;
1-cyclopropyl-7-(6,6-dimethyl-4,5,6,7-tetrahydro-1-benzothien-2-yl)-8-methoxy-4-oxo-1,4-dihydro-3-quinolinecarboxylic acid;
7-(7-amino-6-fluoro-4,5,6,7-tetrahydro-1-benzothien-2-yl)-1-cyclopropyl-8-methoxy-4-oxo-1,4-dihydro-3-quinolinecarboxylic acid;
7-(4-amino-6,6-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-8-methoxy-4-oxo-7-(7-oxo-4,5,6,7-tetrahydro-1,3-benzothiazol-2-yl)-1,4-dihydro-3-quinolinecarboxylic acid;
1-cyclopropyl-7-(6-fluoro-7-hydroxy-4,5,6,7-tetrahydro-1-benzothien-2-yl)-8-methoxy-4-oxo-1,4-dihydro-3-quinolinecarboxylic acid;
1-cyclopropyl-7-(7-hydroxy-6,6-dimethyl-4,5,6,7-tetrahydro-1-benzothien-2-yl)-8-methoxy-4-oxo-1,4-dihydro-3-quinolinecarboxylic acid;
1-cyclopropyl-7-((6S,7S)-7-hydroxy-6-methyl-4,5,6,7-tetrahydro-1-benzothien-2-yl)-8-methoxy-4-oxo-1,4-dihydro-3-quinolinecarboxylic acid;
1-cyclopropyl-8-methoxy-4-oxo-7-(4,5,6,7-tetrahydro-1-benzothien-2-yl)-1,4-dihydro-3-quinolinecarboxylic acid;
7-(7-amino-6,6-difluoro-4,5,6,7-tetrahydro-1-benzothien-2-yl)-1-cyclopropyl-8-methoxy-4-oxo-1,4-dihydro-3-quinolinecarboxylic acid;
7-(7-amino-6,6-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-6-fluoro-8-methoxy-7-(7-(methylamino)-4,5,6,7-tetrahydro-1-benzothien-2-yl)-4-oxo-1,4-dihydro-3-quinolinecarboxylic acid;
1-cyclopropyl-8-methoxy-4-oxo-7-(5-oxo-5,6-dihydro-4H-cyclopenta[b]thien-2-yl)-1,4-dihydro-3-quinolinecarboxylic acid;
1-cyclopropyl-8-methoxy-7-((5E/Z)-5-(methoxyimino)-5,6-dihydro-4H-cyclopenta[b]thien-2-yl)-4-oxo-1,4-dihydro-3-quinolinecarboxylic acid;
7-(5-amino-5,6-dihydro-4H-cyclopenta[b]thien-2-yl)-1-cyclopropyl-8-methoxy-4-oxo-1,4-dihydro-3-quinolinecarboxylic acid;
1-cyclopropyl-7-(5-((ethoxycarbonyl)amino)-5,6-dihydro-4H-cyclopenta[b]thien-2-yl)-8-methoxy-4-oxo-1,4-dihydro-3-quinolinecarboxylic acid;
1-cyclopropyl-8-methoxy-7-(5-((methoxycarbonyl)amino)-5,6-dihydro-4H-cyclopenta[b]thien-2-yI)-4-oxo-1,4-dihydro-3-quinolinecarboxylic acid;
7-(5-(acetylamino)-5,6-dihydro-4H-cyclopenta[b]thien-2-yl)-1-cyclopropyl-8-methoxy-4-oxo-1,4-dihydro-3-quinolinecarboxylic acid;
1-cyclopropyl-8-methoxy-7-(5-(((4-methylphenyl)sulfonyl)amino)-5,6-dihydro-4H-cyclopenta[b]thien-2-yl)-4-oxo-1,4-dihydro-3-quinolinecarboxylic acid;
1-cyclopropyl-8-methoxy-7-(5-((methylsulfonyl)amino)-5,6-dihydro-4H-cyclopenta[b]thien-2-yl)-4-oxo-1,4-dihydro-3-quinolinecarboxylic acid;
7-((5E/Z)-5-((benzyloxy)imino)-5,6-dihydro-4H-cyclopenta[b]thien-2-yl)-1-cyclopropyl-8-methoxy-4-oxo-1,4-dihydro-3-quinolinecarboxylic acid;
1-cyclopropyl-7-((5E/Z)-5-(hydroxyimino)-5,6-dihydro-4H-cyclopenta[b]thien-2-yl)-8-methoxy-4-oxo-1,4-dihydro-3-quinolinecarboxylic acid;
7-(4-amino-4-methyl-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(5-(dimethylamino)-5,6-dihydro-4H-cyclopenta[b]thien-2-yl)-8-methoxy-4-oxo-1,4-dihydro-3-quinolinecarboxylic acid;
7-(4-azido-5,5-dimethyl-4,5,6,7-tetrahydro-1-benzothien-2-yl)-1-cyclopropyl-8-methoxy-4-oxo-1,4-dihydro-3-quinolinecarboxylic acid;
7-(7-amino-4,5 ,6,7-tetrahydro- 1 -benzothien-3-yl)-1-cyclopropyl-8-methoxy-4-oxo-1,4-dihydro-3-quinolinecarboxylic acid;
7-(7-amino-4,5,6,7-tetrahydro- 1 -benzothien-2-yl)-1-cyclopropyl-8-(difluoromethoxy)-4-oxo- 1 ,4-dihydro-3-quinolinecarboxylic acid;
1-cyclopropyl-7-(7-hydroxy-5,5-dimethyl-4,5,6,7-tetrahydro-1-benzothien-2-yl)-8-methoxy-4-oxo-1,4-dihydro-3-quinolinecarboxylic acid;
7-(7-amino-5,5-dimethyl-4,5,6,7-tetrahydro-1-benzothien-2-yl)-1-cyclopropyl-8-methoxy-4-oxo-1,4-dihydro-3-quinolinecarboxylic acid;
1-cyclopropyl-7-(4-hydroxy-4,5,6,7-tetrahydro-1-benzofuran-2-yl)-8-methoxy-4-oxo-1,4-dihydro-3-quinolinecarboxylic acid;
7-(4-amino-4,5,6,7-tetrahydro-1-benzofuran-2-yl)-1-cyclopropyl-8-methoxy-4-oxo-1,4-dihydro-3-quinolinecarboxylic acid;
7-(7-hydroxy-6-spirocyclohexyl-4,5,6,7-tetrahydro-1-benzothien-2-yl)-1-cyclopropyl-8-methoxy-4-oxo-1,4-dihydroquinoline-3-carboxylic acid;
7-(7-amino-5,5-dimethyl-4,5,6,7-tetrahydro-1-benzothien-2-yl)-1-cyclopropyl-8-methoxy-4-oxo-1,4-dihydroquinoline-3-carboxylic acid;
7-(6-(2-aminoethyl)-4,5,6,7-tetrahydro-1-benzothien-2-yl)-1-cyclopropyl-8-methoxy-4-oxo-1,4-dihydroquinoline-3-carboxylic acid;
7-(7-amino-4,5,6,7-tetrahydro-1-benzothien-2-yl)-1-cyclopropyl-6-fluoro-4-oxo- 1,4-dihydro-1,8-naphthyridine-3-carboxylic acid;
1-(2,4-difluorophenyl)-6-fluoro-7-(7-hydroxy-4,5,6,7-tetrahydro-1-benzothien-2-yl)-4-oxo-1,4-dihydro-1,8-naphthyridine-3-carboxylic acid;
7-(4-amino-4,5,6,7-tetrahydro-1-benzothien-2-yl)-1 -(2,4-difluorophenyl)-6-fluoro-4-oxo-1,4-dihydro-1,8-naphthyridine-3-carboxylic acid;
1-(2,4-difluorophenyl)-6-fluoro-7-(4-hydroxy-4,5,6,7-tetrahydro-1-benzothien-2-yl)-4-oxo-1,4-dihydro-1,8-naphthyridine-3-carboxylic acid;
1-(2,4-difluorophenyl)-6-fluoro-7-(4-(methylamino)-4,5,6,7-tetrahydro-1-benzothien-2-yl)-4-oxo-1,4-dihydro-1,8-naphthyridine-3-carboxylic acid;
7-(7-amino-4,5,6,7-tetrahydro-1-benzothien-2-yl)-1-(2,4-difluorophenyl)-6-fluoro-4-oxo-1,4-dihydro-1,8-naphthyridine-3-carboxylic acid;
1-(2,4-difluorophenyl)-6-fluoro-8-methoxy-7-(4-(methylamino)-4,5,6,7-tetrahydro-1-benzothien-2-yl)-4-oxo-1,4-dihydroquinoline-3-carboxylic acid;
1-cyclopropyl-7-(4-(methylamino)-4,5,6,7-tetrahydro-1-benzothien-2-yl)-4-oxo-1,4-dihydroquinoline-3-carboxylic acid;
1-cyclopropyl-7-(7-hydroxy-4,5,6,7-tetrahydro-1-benzothien-2-yl)-4-oxo-1,4-dihydroquinoline-3-carboxylic acid;
1-cyclopropyl-7-(4-hydroxy-4,5,6,7-tetrahydro-1-benzothien-2-yl)-4-oxo-1,4-dihydroquinoline-3-carboxylic acid;
7-(7-amino-4,5 ,6,7-tetrahydro- 1 -benzothien-2-yl)-1-cyclopropyl-4-oxo-1,4-dihydro-1,8-naphthyridine-3-carboxylic acid;
1-(2,4-difluorophenyl)-6-fluoro-7-(4-hydroxy-4,5,6,7-tetrahydro-1-benzothien-2-yl)-8-methoxy-4-oxo-1,4-dihydroquinoline-3-carboxylic acid;
1-cyclopropyl-5-fluoro-7-(4-(methylamino)-4,5,6,7-tetrahydro-1-benzothien-2-yl)-4-oxo-1,4-dihydroquinoline-3-carboxylic acid;
1-(2,4-difluorophenyl)-6-fluoro-4-oxo-7-(4,5,6,7-tetrahydro-1-benzothien-2-yl)-1,4-dihydro-1,8-naphthyridine-3-carboxylic acid;
7-(4-amino-4,5,6,7-tetrahydro- 1 -benzothien-2-yl)-1-cyclopropyl-4-oxo-1,4-dihydroquinoline-3-carboxylic acid;
7-(7-amino-4,5,6,7-tetrahydro- 1 -benzothien-2-yl)-1-cyclopropyl-4-oxo-1,4-dihydroquinoline-3-carboxylic acid;
1-cyclopropyl-5,8-difluoro-7-(4-(methylamino)-4,5,6,7-tetrahydro-1-benzothien-2-yl)-4-oxo-1,4-dihydroquinoline-3-carboxylic acid;
1-cyclopropyl-6-fluoro-7-(4-(methylamino)-4,5,6,7-tetrahydro-1-benzothien-2-yl)-4-oxo-1,4-dihydroquinoline-3-carboxylic acid;
1-cyclopropyl-6,8-difluoro-7-(4-(methylamino)-4,5,6,7-tetrahydro-1-benzothien-2-yl)-4-oxo-1,4-dihydroquinoline-3-carboxylic acid;
1-(2,4-difluorophenyl)-7-(7-hydroxy-4,5,6,7-tetrahydro-1-benzothien-2-yl)-8-methoxy-4-oxo-1,4-dihydroquinoline-3-carboxylic acid;
1-cyclopropyl-6-fluoro-7-(7-hydroxy-4,5,6,7-tetrahydro-1-benzothien-2-yl)-4-oxo-1,4-dihydroquinoline-3-carboxylic acid;
1-cyclopropyl-6,8-difluoro-7-(4-hydroxy-4,5,6,7-tetrahydro-1-benzothien-2-yl)-4-oxo-1,4-dihydroquinoline-3-carboxylic acid;
1-cyclopropyl-6,8-difluoro-7-(7-hydroxy-4,5,6,7-tetrahydro-1-benzothien-2-yl)-4-oxo-1,4-dihydroquinoline-3-carboxylic acid;
1-cyclopropyl-6-fluoro-7-(4-hydroxy-4,5,6,7-tetrahydro-1-benzothien-2-yl)-4-oxo-1,4-dihydroquinoline-3-carboxylic acid;
1-(2,4-difluorophenyl)-7-(4-hydroxy-4,5,6,7-tetrahydro-1-benzothien-2-yl)-8-methoxy-4-oxo-1,4-dihydroquinoline-3-carboxylic acid;
1-(2,4-difluorophenyl)-6-fluoro-7-(7-hydroxy-4,5,6,7-tetrahydro-1-benzothien-2-yl)-8-methoxy-4-oxo-1,4-dihydroquinoline-3-carboxylic acid;
1-cyclopropyl-6-fluoro-7-(7-hydroxy-4,5,6,7-tetrahydro-1-benzothien-2-yl)-8-methoxy-4-oxo-1,4-dihydroquinoline-3-carboxylic acid;
1-cyclopropyl-5,8-difluoro-7-(4-hydroxy-4,5,6,7-tetrahydro-1-benzothien-2-yl)-4-oxo-1,4-dihydroquinoline-3-carboxylic acid;
1-cyclopropyl-5,8-difluoro-7-(7-hydroxy-4,5,6,7-tetrahydro-1-benzothien-2-yl)-4-oxo-1,4-dihydroquinoline-3-carboxylic acid;
1-cyclopropyl-7-(7-((4-fluorobenzyl)amino)-4,5,6,7-tetrahydro-1-benzothien-2-yl)-8-methoxy-4-oxo-1,4-dihydroquinoline-3-carboxylic acid;
1-cyclopropyl-5-fluoro-7-(4-hydroxy-4,5 ,6,7-tetrahydro-1-benzothien-2-yl)-4-oxo-1,4-dihydroquinoline-3-carboxylic acid;
1-(2,4-difluorophenyl)-8-methoxy-7-(4-(methylamino)-4,5,6,7-tetrahydro-1-benzothien-2-yl)-4-oxo-1,4-dihydroquinoline-3-carboxylic acid;
7-(4-amino-4,5,6,7-tetrahydro-1-benzothien-2-yl)-1-(2,4-difluorophenyl)-6-fluoro-8-methoxy-4-oxo-1,4-dihydroquinoline-3-carboxylic acid;
1-(2,4-difluorophenyl)-7-(4-(methylamino)-4,5,6,7-tetrahydro-1-benzothien-2-yl)-4-oxo-1,4-dihydro-1,8-naphthyridine-3-carboxylic acid;
7-(4-amino-4,5,6,7-tetrahydro-1-benzothien-2-yl)-1-cyclopropyl-6-fluoro-4-oxo-1,4-dihydroquinoline-3-carboxylic acid;
7-(4-amino-4,5,6,7-tetrahydro-1-benzothien-2-yl)-1-cyclopropyl-6,8-difluoro-4-oxo-1,4-dihydroquinoline-3-carboxylic acid;
7-(7-amino-4,5,6,7-tetrahydro-1-benzothien-2-yl)-1-cyclopropyl-6,8-difluoro-4-oxo-1,4-dihydroquinoline-3-carboxylic acid;
7-(7-amino-4,5,6,7-tetrahydro-1-benzothien-2-yl)-1-cyclopropyl-6-fluoro-4-oxo-1,4-dihydroquinoline-3-carboxylic acid;
1-cyclopropyl-7-(7-hydroxy-4,5 ,6,7-tetrahydro-1-benzothien-2-yl)-6-methyl-4-oxo-1,4-dihydroquinoline-3-carboxylic acid;
1-cyclopropyl-6-methyl-7-(4-(methylamino)-4,5,6,7-tetrahydro-1-benzothien-2-yl)-4-oxo-1,4-dihydroquinoline-3-carboxylic acid;
7-(7-amino-4,5,6,7-tetrahydro- 1 -benzothien-2-yl)-1-(2,4-difluorophenyl)-8-methoxy-4-oxo-1,4-dihydroquinoline-3-carboxylic acid;
8-chloro-1-cyclopropyl-7-(4-hydroxy-4,5,6,7-tetrahydro-1-benzothien-2-yl)-4-oxo-1,4-dihydroquinoline-3-carboxylic acid;
8-chloro-1-cyclopropyl-7-(7-hydroxy-4,5,6,7-tetrahydro-1-benzothien-2-yl)-4-oxo-1,4-dihydroquinoline-3-carboxylic acid;
1-cyclopropyl-6-fluoro-7-(4-hydroxy-4,5,6,7-tetrahydro-1-benzothien-2-yl)-8-methyl-4-oxo-1,4-dihydroquinoline-3-carboxylic acid;
1-cyclopropyl-6-fluoro-8-methyl-7-(4-(methylamino)-4,5,6,7-tetrahydro-1-benzothien-2-yl)-4-oxo-1,4-dihydroquinoline-3-carboxylic acid;
7-(4-amino-4,5,6,7-tetrahydro-1-benzothien-2-yl)-1-(2,4-difluorophenyl)-8-methoxy-4-oxo-1,4-dihydroquinoline-3-carboxylic acid;
5-amino-1-cyclopropyl-6, 8-difluoro-7-(7-hydroxy-4,5,6,7-tetrahydro-1-benzothien-2-yl)-4-oxo-1,4-dihydroquinoline-3-carboxylic acid;
1-cyclopropyl-6-fluoro-7-(7-hydroxy-4,5,6,7-tetrahydro-1-benzothien-2-yl)-4-oxo-1,4-dihydro-1,8-naphthyridine-3-carboxylic acid;
7-(4-amino-4,5 ,6,7-tetrahydro-1-benzothien-2-yl)-1-cyclopropyl-6-fluoro-8-methyl-4-oxo-1,4-dihydroquinoline-3-carboxylic acid;
1-cyclopropyl-7-(7-hydroxy-4,5,6,7-tetrahydro-1-benzothien-2-yl)-6-methyl-4-oxo-1,4-dihydro-1,8-naphthyridine-3-carboxylic acid;
7-(4-amino-4,5,6,7-tetrahydro-1-benzothien-2-yl)-1-cyclopropyl-5,8-difluoro-4-oxo-1,4-dihydroquinoline-3-carboxylic acid;
7-(7-amino-4,5,6,7-tetrahydro-1-benzothien-2-yl)-1-cyclopropyl-5,8-difluoro-4-oxo-1,4-dihydroquinoline-3-carboxylic acid;
7-(7-amino-4,5,6,7-tetrahydro-1-benzothien-2-yl)-1-cyclopropyl-6-methyl-4-oxo-1,4-dihydroquinoline-3-carboxylic acid;
7-(4-amino-4,5,6,7-tetrahydro-1-benzothien-2-yl)-8-chloro-1-cyclopropyl-4-oxo-1,4-dihydroquinoline-3-carboxylic acid;
7-(7-amino-4,5,6,7-tetrahydro-1-benzothien-2-yl)-8-chloro-1-cyclopropyl-4-oxo-1,4-dihydroquinoline-3-carboxylic acid;
5-amino-7-(4-amino-4,5,6,7-tetrahydro-1-benzothien-2-yl)-1-cyclopropyl-6,8-difluoro-4-oxo-1,4-dihydroquinoline-3-carboxylic acid;
7-(7-amino-4,5,6,7-tetrahydro-1-benzothien-2-yl)-1-cyclopropyl-6-methyl-4-oxo-1,4-dihydro-1,8-naphthyridine-3-carboxylic acid;
7-(7-(benzylamino)-4,5,6,7-tetrahydro-1-benzothien-2-yl)-1-cyclopropyl-8-methoxy-4-oxo-1,4-dihydroquinoline-3-carboxylic acid;
1-cyclopropyl-8-methoxy-4-oxo-7-(7-((pyridin-3-ylmethyl)amino)-4,5,6,7-tetrahydro-1-benzothien-2-yl)-1,4-dihydroquinoline-3-carboxylic acid;
7-(4-amino-4,5,6,7-tetrahydro-1-benzothien-2-yl)-1-cyclopropyl-6-methyl-4-oxo-1,4-dihydroquinoline-3-carboxylic acid;
1-cyclopropyl-7-(4-hydroxy-4,5,6,7-tetrahydro-1-benzothien-2-yl)-6-methyl-4-oxo-1,4-dihydroquinoline-3-carboxylic acid;
7-(4-amino-4,5,6,7-tetrahydro-1-benzothien-2-yl)-1-cyclopropyl-6-methyl-4-oxo-1,4-dihydro-1,8-naphthyridine-3-carboxylic acid;
7-(7-amino-6,6-dimethyl-4,5,6,7-tetrahydro-1-benzothien-2-yl)-1-cyclopropyl-6-fluoro-4-oxo-1,4-dihydroquinoline-3-carboxylic acid;
1-cyclopropyl-6-fluoro-7-(7-hydroxy-6,6-dimethyl-4,5,6,7-tetrahydro-1-benzothien-2-yl)-4-oxo-1,4-dihydroquinoline-3-carboxylic acid;
7-(7-amino-6,6-dimethyl-4,5,6,7-tetrahydro-1-benzothien-2-yl)-1-cyclopropyl-6-fluoro-4-oxo-1,4-dihydro-1,8-naphthyridine-3-carboxylic acid;
1-cyclopropyl-6-fluoro-7-(7-hydroxy-6,6-dimethyl-4,5,6,7-tetrahydro-1-benzothien-2-yl)-4-oxo-1,4-dihydro-1,8-naphthyridine-3-carboxylic acid;
5-amino-7-(7-amino-4,5,6,7-tetrahydro-1-benzothien-2-yl)-1-cyclopropyl-6,8-difluoro-4-oxo-1,4-dihydroquinoline-3-carboxylic acid;
5-amino-1-cyclopropyl-6,8-difluoro-7-(4-(methylamino)-4,5,6,7-tetrahydro-1-benzothien-2-yl)-4-oxo-1,4-dihydroquinoline-3-carboxylic acid;
7-(7-amino-6,6-dimethyl-4,5,6,7-tetrahydro-1-benzothien-2-yl)-8-chloro-1-cyclopropyl-4-oxo-1,4-dihydroquinoline-3-carboxylic acid;
7-(7-amino-6,6-dimethyl-4,5,6,7-tetrahydro-1-benzothien-2-yl)-1-cyclopropyl-6-fluoro-8-methoxy-4-oxo-1,4-dihydroquinoline-3-carboxylic acid;
5-amino-7-(7-amino-4,5,6,7-tetrahydro-1-benzothien-2-yl)-1-cyclopropyl-8-fluoro-4-oxo-1,4-dihydroquinoline-3-carboxylic acid;
5-amino-i -cyclopropyl-8-fluoro-7-(7-hydroxy-4,5 ,6,7-tetrahydro- 1 -benzothien-2-yl)-4-oxo-1,4-dihydroquinoline-3-carboxylic acid;
5-amino-1-cyclopropyl-8-fluoro-7-(4-hydroxy-4,5,6,7-tetrahydro- 1 -benzothien-2-yl)-4-oxo- 1,4-dihydroquinoline-3-carboxylic acid;
5-amino-1-cyclopropyl-7-(4-hydroxy-4,5,6,7-tetrahydro-1-benzothien-2-yl)-4-oxo-1,4-dihydroquinoline-3-carboxylic acid;
5-amino-7-(7-amino-4,5,6,7-tetrahydro-1-benzothien-2-yl)-6-chloro-1-cyclopropyl-8-fluoro-4-oxo-1,4-dihydroquinoline-3-carboxylic acid;
5-amino-6-chloro-1-cyclopropyl-8-fluoro-7-(7-hydroxy-4,5 ,6,7-tetrahydro-1-benzothien-2-yl)-4-oxo-1,4-dihydroquinoline-3-carboxylic acid;
5-amino-6-chloro-1-cyclopropyl-8-fluoro-7-(4-hydroxy-4,5,6,7-tetrahydro-1-benzothien-2-yl)-4-oxo-1,4-dihydroquinoline-3-carboxylic acid;
8-chloro-1-cyclopropyl-6-fluoro-7-(7-hydroxy-4,5,6,7-tetrahydro-1-benzothien-2-yl)-4-oxo-1,4-dihydroquinoline-3-carboxylic acid;
8-chloro-1-cyclopropyl-6-fluoro-7-(4-(methylamino)-4,5,6,7-tetrahydro-1-benzothien-2-yl)-4-oxo-1,4-dihydroquinoline-3-carboxylic acid;
8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid;
8-chloro-1-cyclopropyl-7-(4-(methylamino)-4,5,6,7-tetrahydro-1-benzothien-2-yl)-4-oxo-1,4-dihydroquinoline-3-carboxylic acid;
7-(7-amino-4,5,6,7-tetrahydro-1-benzothien-2-yl)-1-cyclopropyl-8-(difluoromethoxy)-6-fluoro-4-oxo-1,4-dihydroquinoline-3-carboxylic acid;
1-cyclopropyl-8-(difluoromethoxy)-6-fluoro-7-(7-hydroxy-6,6-dimethyl-4,5,6,7-tetrahydro-1-benzothien-2-yl)-4-oxo-1,4-dihydroquinoline-3-carboxylic acid;
1-cyclopropyl-6-fluoro-7-(4-hydroxy-4,5,6,7-tetrahydro-1-benzothien-2-yl)-5-methyl-4-oxo-1,4-dihydro-1,8-naphthyridine-3-carboxylic acid;
7-(4-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid;
7-(7-amino-4,5,6,7-tetrahydro-1-benzothien-2-yl)-1-cyclopropyl-6-fluoro-8-methyl-4-oxo-1,4-dihydroquinoline-3-carboxylic acid;
1-cyclopropyl-6-fluoro-7-(7-hydroxy-4,5,6,7-tetrahydro-1-benzothien-2-yl)-8-methyl-4-oxo-1,4-dihydroquinoline-3-carboxylic acid;
1-cyclopropyl-6-fluoro-7-(7-hydroxy-4,5,6,7-tetrahydro-1-benzothien-2-yl)-5-methyl-4-oxo-1,4-dihydro-1,8-naphthyridine-3-carboxylic acid;
7-(7-amino-4,5,6,7-tetrahydro-1-benzothien-2-yl)-1-cyclopropyl-6-fluoro-5-methyl-4-oxo-1,4-dihydro-1,8-naphthyridine-3-carboxylic acid;
7-(7-amino-6,6-dimethyl-4,5,6,7-tetrahydro-1 -benzothien-2-yl)-1-cyclopropyl-6-fluoro-8-methyl-4-oxo-1,4-dihydroquinoline-3-carboxylic acid;
7-(7-amino-6,6-dimethyl-4,5,6,7-tetrahydro-1-benzothien-2-yl)- 1 -cyclopropyl-6,8-difluoro-4-oxo-1,4-dihydroquinoline-3-carboxylic acid;
7-(7-amino-4,5,6,7-tetrahydro-1-benzothien-2-yl)-1-cyclopropyl-8-methyl-4-oxo-1,4-dihydroquinoline-3-carboxylic acid;
1-cyclopropyl-7-(7-hydroxy-4,5,6,7-tetrahydro-1-benzothien-2-yl)-8-methyl-4-oxo-1,4-dihydroquinoline-3-carboxylic acid;
7-(7-amino-6,6-dimethyl-4,5,6,7-tetrahydro-1-benzothien-2-yl)-1-cyclopropyl-8-methyl-4-oxo-1,4-dihydroquinoline-3-carboxylic acid;
1-cyclopropyl-7-(7-hydroxy-6,6-dimethyl-4,5,6,7-tetrahydro-1-benzothien-2-yl)-8-methyl-4-oxo-1,4-dihydroquinoline-3-carboxylic acid;
7-(4-amino-4,5,6,7-tetrahydro-1-benzothien-2-yl)-1-cyclopropyl-8-methyl-4-oxo-1,4-dihydroquinoline-3-carboxylic acid;
1-cyclopropyl-7-(4-hydroxy-4,5,6,7-tetrahydro-1-benzothien-2-yl)-8-methyl-4-oxo-1,4-dihydroquinoline-3-carboxylic acid;
1-cyclopropyl-8-methyl-7-[4-(methylamino)-4,5,6,7-tetrahydro-1-benzothien-2-yl]-4-oxo-1,4-dihydroquinoline-3-carboxylic acid;
7-(7-amino-4,5,6,7-tetrahydro-1-benzothien-2-yl)-1-cyclopropyl-6-fluoro-5,8-dimethyl-4-oxo-1,4-dihydroquinoline-3-carboxylic acid;
1-cyclopropyl-6-fluoro-7-(7-hydroxy-4,5,6,7-tetrahydro- 1 -benzothien-2-yl)-5,8-dimethyl-4-oxo-1,4-dihydroquinoline-3-carboxylic acid;
5-amino-7-(4-amino-4,5,6,7-tetrahydro-1-benzothien-2-yl)-6-chloro-1-cyclopropyl-8-fluoro-4-oxo-1,4-dihydroquinoline-3-carboxylic acid;
5-amino-7-(7-amino-6,6-dimethyl-4,5,6,7-tetrahydro-1-benzothien-2-yl)-6-chloro-1-cyclopropyl-8-fluoro-4-oxo-1,4-dihydroquinoline-3-carboxylic acid;
5-amino-7-(4-amino-4,5,6,7-tetrahydro-1-benzothien-2-yl)-8-chloro-1-cyclopropyl-6-fluoro-4-oxo-1,4-dihydroquinoline-3-carboxylic acid;
5-amino-7-(7-amino-4,5,6,7-tetrahydro-1-benzothien-2-yl)-8-chloro-1-cyclopropyl-6-fluoro-4-oxo-1,4-dihydroquinoline-3-carboxylic acid;
5-amino-7-(7-amino-6,6-dimethyl-4,5,6,7-tetrahydro- 1 -benzothien-2-yl)-8-chloro- 1-cyclopropyl-6-fluoro-4-oxo-1,4-dihydroquinoline-3-carboxylic acid;
5-amino-8-chloro-1-cyclopropyl-6-fluoro-7-(4-hydroxy-4,5,6,7-tetrahydro-1-benzothien-2-yl)-4-oxo-1,4-dihydroquinoline-3-carboxylic acid;
5-amino-8-chloro-1-cyclopropyl-6-fluoro-7-(7-hydroxy-4,5 ,6,7-tetrahydro- 1-benzothien-2-yl)-4-oxo-1,4-dihydroquinoline-3-carboxylic acid;
7-(4-amino-4,5,6,7-tetrahydro-1-benzothien-2-yl)-8-chloro-1-cyclopropyl-6-fluoro-4-oxo-1,4-dihydroquinoline-3-carboxylic acid;
7-(7-amino-4,5,6,7-tetrahydro-1-benzothien-2-yl)-8-chloro-1-cyclopropyl-6-fluoro-4-oxo-1,4-dihydroquinoline-3-carboxylic acid;
7-(7-amino-6,6-dimethyl-4,5,6,7-tetrahydro-1-benzothien-2-yl)-8-chloro- 1-cyclopropyl-6-fluoro-4-oxo-1,4-dihydroquinoline-3-carboxylic acid;
5-amino-7-(7-amino-6,6-dimethyl-4,5,6,7-tetrahydro-1-benzothien-2-yl)-1-cyclopropyl-6,8-difluoro-4-oxo-1,4-dihydroquinoline-3-carboxylic acid;
5-amino-1-cyclopropyl-6-fluoro-7-(4-hydroxy-4,5,6,7-tetrahydro-1-benzothien-2-yl)-8-methyl-4-oxo-1,4-dihydroquinoline-3-carboxylic acid;
5-amino-7-(4-amino-4,5,6,7-tetrahydro-1-benzothien-2-yl)-1-cyclopropyl-6-fluoro-8-methyl-4-oxo-1,4-dihydroquinoline-3-carboxylic acid;
5-amino-1-cyclopropyl-6-fluoro-8-methyl-7-[4-(methylamino)-4,5,6,7-tetrahydro-1-benzothien-2-yl]-4-oxo-1,4-dihydroquinoline-3-carboxylic acid;
5-amino-7-(7-amino-4,5,6,7-tetrahydro-1-benzothien-2-yl)-1-cyclopropyl-6-fluoro-8-methyl-4-oxo-1,4-dihydroquinoline-3-carboxylic acid;
5-amino-1-cyclopropyl-6-fluoro-7-(7-hydroxy-4,5,6,7-tetrahydro-1-benzothien-2-yl)-8-methyl-4-oxo-1,4-dihydroquinoline-3-carboxylic acid;
5-amino-7-(7-amino-6,6-dimethyl-4,5,6,7-tetrahydro-1-benzothien-2-yl)-1-cyclopropyl-6-fluoro-8-methyl-4-oxo-1,4-dihydroquinoline-3-carboxylic acid;
5-amino-1-cyclopropyl-7-(4-hydroxy-4,5,6,7-tetrahydro-1-benzothien-2-yl)-8-methyl-4-oxo-1,4-dihydroquinoline-3-carboxylic acid;
5-amino-7-(4-amino-4,5,6,7-tetrahydro-1-benzothien-2-yl)-1-cyclopropyl-8-methyl-4-oxo-1,4-dihydroquinoline-3-carboxylic acid;
5-amino-1-cyclopropyl-8-methyl-7-[4-(methylamino)-4,5,6,7-tetrahydro-1-benzothien-2-yl]-4-oxo-1,4-dihydroquinoline-3-carboxylic acid;
5-amino-1-cyclopropyl-7-(7-hydroxy-4,5,6,7-tetrahydro-1-benzothien-2-yl)-8-methyl-4-oxo-1,4-dihydroquinoline-3-carboxylic acid;
5-amino-7-(7-amino-4,5,6,7-tetrahydro-1-benzothien-2-yl)-1-cyclopropyl-8-methyl-4-oxo-1,4-dihydroquinoline-3-carboxylic acid;
and
5-amino-7-(7-amino-6,6-dimethyl-4,5,6,7-tetrahydro-1-benzothien-2-yl)-1-cyclopropyl-8-methyl-4-oxo-1,4-dihydroquinoline-3-carboxylic acid.

24. A compound selected from the group consisting of compounds of formula (Im) 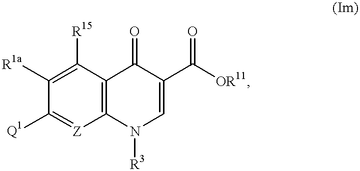
or pharmaceutically acceptable salts or prodrugs thereof, wherein
R^{1a} is optionally substituted alkyl;
R¹⁵ is independently selected from the group consisting of
(1) hydrogen,
(2) optionally substituted alkyl,
(3) halide,
(4) nitro,
and
(5) optionally protected amino;
Q¹ is selected from the group consisting of halide, methanesulfonate, and trifluoromethanesulfonate;
Z is nitrogen or 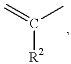
wherein R² is selected from the group consisting of
(1) hydrogen,
(2) optionally substituted alkyl,
(3) halide,
(4) optionally protected hydroxyl,
(5) —OR⁸,
and
(6) —S(O)ₙR⁸, wherein n is zero, one, or two,
and
wherein R⁸ in (4) and (5) is selected from the group consisting of
(a) C₃-C₆ alkenyl,
(b) C₁-C₆ alkyl,
and
(c) C₃-C₆ alkynyl,
wherein (a)-(c) can be optionally substituted with one, two, or three substituents independently selected from the group consisting of
(i) alkoxy,
(ii) aryl,
(iii) C₃-C₆ cycloalkyl,
(iv) azido,
(v) cyano,
(vi) halide,
(vii) optionally protected amino,
(viii) optionally protected carboxyl,
and
(ix) optionally protected hydroxyl;
R³ is selected from the group consisting of
(1) C₃-C₆ alkenyl,
(2) C₁-C₆ alkyl,
(3) C₃-C₆ alkynyl,
wherein (1)-(3) can be optionally substituted with one, two, or three substutuents independently selected from the group consisting of
(a) C₁-C₆ alkanoyloxy,
(b) C₁-C₆ alkoxy,
(c) aryl,
(d) azido,
(e) cyano,
(f) C₃-C₆ cycloalkyl,
(g) halide,
(h) optionally protected amino,
(i) optionally protected carboxyl,
(j) optionally protected hydroxyl,
(k) oxo,
(l) C₁-C₆ perfluoroalkoxy,
(m) C₁-C₆ perfluorothioalkoxy,
and
(n) thioxo,
(4) aryl,
(5) C₃-C₆ cycloalkyl,
and
(6) heterocycle,
wherein (4)-(6) can be optionally substituted with one, two, or three substituents independently selected from the group consisting of
(a) C₁-C₆ alkanoyloxy,
(b) C₁-C₆ alkoxy,
(c) C₂-C₆ alkenyl,
(d) C₁-C₆ alkyl,
(e) C₂-C₆ alkynyl,
(f) aryl,
(g) azido,
(h) cyano,
(i) C₃-C₆ cycloalkyl,
(j) halide,
(k) optionally protected amino
(l) optionally protected carboxyl,
(m) optionally protected hydroxyl,
(n) C₁-C₆ perfluoroalkoxy,
and
(o) C₁-C₆ perfluorothioalkoxy; and
R¹¹ is hydrogen or a carboxyl protecting group.

25. A compound according to claim 24 wherein R^{1a} is C₁-C₆ alkyl.

26. A compound according to claim 25 wherein R^{1a} is methyl.

27. A compound according to claim 24 wherein R¹⁵ is hydrogen.

28. A compound according to claim 24 wherein Q¹ is chloride.

29. A compound according to claim 24 wherein Q¹ is bromide.

30. A compound according to claim 24 wherein R³ is cyclopropyl.

31. A compound according to claim 24 wherein Z is 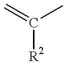
wherein R² is hydrogen.

32. A compound according to claim 24 wherein Z is nitrogen.

33. A compound according to claim 24 which is ethyl 7-bromo-1-cyclopropyl-6-methyl-4-oxo-1,4-dihydroquinoline-3-carboxylate.

34. A compound according to claim 24 which is ethyl 7-chloro-1-cyclopropyl-6-methyl-4-oxo- 1,4-dihydro-1,8-naphthyridine-3-carboxylate.
